SEQUENCE LISTING (7 sequences)

SEQ ID NO: 1 - protein, 414 aa, human
  MISC_FEATURE (1)..(414): GST-hK-RasG12C
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDPITSLYKKAGSDYDIPTTENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 533 aa, human
  MISC_FEATURE (1)..(533): His10-hSOS1
MGHHHHHHHHHHSSGHIEGRHMLETSLYKKAGSDYDIPTTENLYFQGEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 3 - protein, 536 aa, human
  MISC_FEATURE (1)..(536): His10-hSOS1_12
MGHHHHHHHHHHSSGHIEGRHMLETSLYKKAGSDYDIPTTENLYFQGAMAEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 4 - protein, 490 aa, human
  MISC_FEATURE (1)..(490): hSOS1_12 (tag-free)
GAMAEEQMRLPSADVYRFAEPDSEENIIFEENMQPKAGIPIIKAGTVIKLIERLTYHMYADPNFVRTFLTTYRSFCKPQELLSLIIERFEIPEPEPTEADRIAIENGDQPLSAELKRFRKEYIQPVQLRVLNVCRHWVEHHFYDFERDAYLLQRMEEFIGTVRGKAMKKWVESITKIIQRKKIARDNGPGHNITFQSSPPTVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYSYPLKSPGVRPSNPRPGT

SEQ ID NO: 5 - protein, 527 aa, human
  MISC_FEATURE (1)..(527): His10-hSOS2
MGHHHHHHHHHHSSGHIEGRHMLETSLYKKAGSDYDIPTTENLYFQGPLRLPSPEVYRFVVKDSEENIVFEDNLQSRSGIPIIKGGTVVKLIERLTYHMYADPNFVRTFLTTYRSFCKPQELLSLLIERFEIPEPEPTDADKLAIEKGEQPISADLKRFRKEYVQPVQLRILNVFRHWVEHHFYDFERDLELLERLESFISSVRGKAMKKWVESIAKIIRRKKQAQANGVSHNITFESPPPPIEWHISKPGQFETFDLMTLHPIEIARQLTLLESDLYRKVQPSELVGSVWTKEDKEINSPNLLKMIRHTTNLTLWFEKCIVEAENFEERVAVLSRIIEILQVFQDLNNFNGVLEIVSAVNSVSVYRLDHTFEALQERKRKILDEAVELSQDHFKKYLVKLKSINPPCVPFFGIYLTNILKTEEGNNDFLKKKGKDLINFSKRRKVAEITGEIQQYQNQPYCLRIEPDMRRFFENLNPMGSASEKEFTDYLFNKSLEIEPRNCKQPPRFPRKSTFSLKSPGIRPNTG

SEQ ID NO: 6 - protein, 536 aa, human; identical to SEQ ID NO: 3
  MISC_FEATURE (1)..(536): hSOS1_12 with N-terminal His tag (His10-hSOS1_12) before cleavage by TEV protease

SEQ ID NO: 7 - protein, 490 aa, human; identical to SEQ ID NO: 4
  MISC_FEATURE (1)..(490): hSOS1_12 after cleavage by TEV protease

(19) United States
(12) Patent Application Publication
(10) Pub. No.: US 2023/0029385 A1
(43) Pub. Date: Jan. 26, 2023

(54) 2-METHYL-AZA-QUINAZOLINES

(71) Applicant: Bayer Aktiengesellschaft, Leverkusen (DE)

(72) Inventors: Lars WORTMANN, Biberbach an der Riss (DE); Keith GRAHAM, Berlin (DE); Benjamin BADER, Berlin (DE); Roman HILLIG, Berlin (DE); Jens SCHROEDER, Berlin (DE); Philip LIENAU, Berlin (DE); Hans BRIEM, Berlin (DE)

(21) Appl. No.: 17/768,684

(22) PCT Filed: Oct. 14, 2020
(86) PCT No.: PCT/EP2020/078908

§ 371 Date: Apr. 13, 2022

(30) Foreign Application Priority Data
Oct. 15, 2019 (EP) 19203282.9
Dec. 20, 2019 (EP) 19218861.3

(51) Int. Cl. C07D 471/04; C07D 519/00

(52) U.S. Cl. CPC C07D 471/04; C07D 519/00

(57) ABSTRACT

The present invention covers 2-methyl-aza-quinazoline compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients.

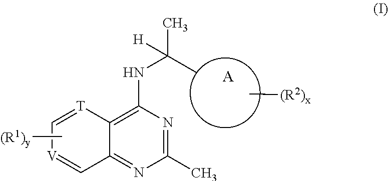

The present invention covers 2-methyl-aza-quinazoline compounds of general formula (I) as described and defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients.

### BACKGROUND

The present invention covers 2-methyl-aza-quinazoline compounds of general formula (I) which inhibit the Ras-Sos1 interaction.

US 2011/0054173 A1 discloses certain 1- or 2-(4-(aryloxy)-phenyl)ethylamino-, oxy- or sulfanyl)pteridines and 1- or 2-(4-(heteroaryloxy)-phenyl)ethylamino-, oxy- or sulfanyl)pteridines and their use as agrochemicals and animal health products.

In the 2-position substituted quinazoline compounds are described e.g. in EP 0326328, EP 0326329, WO93/007124, WO2003/087098 and U.S. Pat. No. 5,236,925. These compounds are either not described as pharmaceutically active compounds or, if they are described as pharmacologically active compounds, they are described as compounds having affinity to the Epidermal Growth Factor Receptor (EGFR).

In the majority (45-100%) of patients receiving EGFR inhibitors skin toxicity is a class-specific side effect that is typically manifested as a papulopustular rash. The skin toxicity is related to the inhibition of EGFR in the skin, which is crucial for the normal development and physiology of the epidermis.

However, the state of the art does not describe:
the 2-methyl substituted quinazoline compounds of general formula (I) of the present invention as described and defined herein, i.e. compounds having a quinazoline core bearing a methyl group on the carbon atom 2 which effectively and selectively inhibit the Ras-Sos1 interaction without significantly targeting the EGFR receptor.

Ras proteins play an important role in human cancer. Mutations in Ras proteins can be found in 20-30% of all human tumors and are recognized as tumorigenic drivers especially in lung, colorectal and pancreatic cancers (Malumbres & Barbacid 2002 Nature Reviews Cancer, Pylayeva-Gupta et al. 2011 Nature Reviews Cancer). Three human Ras genes are known that encode four different Ras proteins of 21 kDa size: H-Ras, N-Ras, and two splice variants of K-Ras, namely K-Ras 4A and K-Ras-4B. All Ras isoforms are highly conserved within the GTP-binding domain and differ mainly in the hypervariable C-terminal region. The C-termini of the different Ras-isoforms are posttranslationally modified by lipidation (farnesylation, palmitoylation) to facilitate membrane anchorage. The localization of Ras-proteins at the cytoplasmic membrane provides vicinity to transmembrane growth receptors and has been shown to be essential for transmitting growth signals from extracellular growth factor binding to intracellular downstream pathways. A variety of upstream signals may activate Ras proteins depending on the cellular context, such as epidermal growth factor receptor (EGFR), platelet-derived growth factor receptor (PDGFR), nerve growth factor receptor (NGFR) and others. Activated Ras can signal through various downstream pathways, e.g. the Raf-MEK-ERK or the PI3K-PDK1-Akt pathways.

On the molecular level, Ras proteins function as molecular switches. By binding GTP and GDP they exist in an active (GTP-bound) and inactive (GDP-bound) state in the cell. Active GTP-loaded Ras recruits other proteins by binding of their cognate Ras-binding domains (RBDs) resulting in activation of the effector protein followed by downstream signalling events of diverse functions, e.g. cytoskeletal rearrangements or transcriptional activation. The activity status of Ras is tightly regulated by guanine nucleotide exchange factors (GEFs) and GTPase activating proteins (GAPs). GEFs function as activators of Ras by promoting the nucleotide exchange from GDP to GTP. GAPs deactivate Ras-GTP by catalyzing the hydrolysis of the bound GTP to GDP. In a cancer cell, point mutations, typically within the GTP-binding region at codon 12, eliminate the ability of RAS to efficiently hydrolyse bound GTP, even in the presence of a GAP. Therefore, cancer cells comprise increased levels of active mutated Ras-GTP, which is thought to be a key factor for driving cancer cell proliferation.

Three main families of RAS-specific GEFs have been identified so far (reviewed in Vigil 2010 Nature Reviews Cancer; Rojas et al 2011, Genes & Cancer 2(3) 298-305). There are two son of sevenless proteins (SOS1 and SOS2), 4 different isoforms of Ras guanine nucleotide releasing proteins (Ras-GRP1-4) and two Ras guanine nucleotide releasing factors (Ras-GRF1 and 2). The SOS proteins are ubiquitously expressed and are recruited to sites of activated growth factors. Ras-GRFs are expressed mainly in the nervous system, where they are involved in Calcium-dependent activation of Ras. In contrast, Ras GRP proteins are expressed in hematopoietic cells and act in concert with non-receptor tyrosine kinases. In the context of cancer, mainly SOS proteins have been found to be involved.

Targeting Ras for cancer therapy has been a dream since the 1990s (Downward 2002 Nature Reviews Cancer, Krens et al. 2010 Drug Discovery Today). Due to the compact nature, the high affinity towards GDP and GTP in combination with high intracellular GTP concentrations, the Ras protein itself has always been considered to be undruggable, i.e. the chance to identify small chemical molecules that would bind to and inhibit active Ras was rated extremely low. Alternative approaches have been undertaken to reduce Ras signaling, e.g. by addressing more promising drug targets such as enzymes involved in the posttranslational modification of Ras proteins, especially farnesyltransferase and geranylgeranyltransferase (Berndt 2011 Nature Reviews Cancer). Inhibitors of farnesyltransferase (FTIs) were identified and developed with promising antitumor effects in preclinical models. Unexpectedly, in clinical trials these inhibitors have been of limited efficacy. Targeting upstream and downstream kinases involved in Ras signaling pathways has been more successful. Several drugs are and have been in clinical trials that inhibit different kinases, e.g. EGFR, Raf, MEK, Akt, PI3K (Takashima & Faller 2013 Expert Opin. Ther. Targets). Marketed cancer drugs are available that inhibit Raf, EGFR or MEK.

Nevertheless, there is still a large unmet need for the treatment of Ras-dependent tumors that are resistant against current therapies. Many research groups have been active to identify small molecules that target Ras directly (Ras small molecules have been reviewed in: Cox et al. 2014 Nature Reviews Drug Discovery, Spiegel et al. 2014 Nature Chemical Biology, Cromm 2015 Angewandte Chemie, Marin-Ramos et al Seminars in Cancer Biology). One group of inhibitors comprises small molecules that inhibit the interaction of Ras with its effectors Raf or PI3K. Another group of compounds acts as covalent inhibitors of a specific cysteine mutant form of K-Ras (glycine to cysteine point mutation G12C). The specific targeting of the Ras-G12C mutant might have the benefit of reduced side effects, as the wildtype Ras proteins should not be affected. Furthermore, several reports show small molecules and peptides that interrupt the GEF assisted activation of Ras (Hillig et al 2019 PNAS; Gray et al 2019 Angewandte Chemie). There seem to be several different binding sites possible that result in this mode of action. Inhibitors may bind to Ras or to the GEF in an allosteric or orthosteric fashion. All these approaches of direct Ras-targeting are in preclinical research stage. Stabilized peptides have been shown to be active in the nanomolar range. (Leshchiner et al. 2015 PNAS). Their usefulness as drugs in a clinical setting has to be awaited.

The Epidermal Growth Factor Receptor (EGFR) is a tyrosine kinase (TK) receptor that is activated upon binding to the Epidermal Growth Factor and other growth factor ligands, triggering several downstream pathways, including RAS/MAPK, PI3K/Akt and STAT that regulate different cellular processes, including DNA synthesis and proliferation (Russo A, Oncotarget.4254, 2015). The family of HER (ErbB) receptor tyrosine kinases consists of four members, ie, epidermal growth factor receptors [EGFR (HER1 or ErbB1), HER2 (ErbB2, neu), HER3 (ErbB3), and HER4 (ErbB4)]. Overexpression, mutation, or aberrant activity of these receptors has been implicated in various types of cancer (Feldinger K, Breast Cancer (Dove Med Press), 2015, 7, 147).

First-Generation Inhibitors

Erlotinib and Gefitinib are small molecule inhibitors of the EGFR/HER-1 (human epidermal growth factor receptor) tyrosine kinase. Erlotinib and Gefitinib were developed as reversible and highly specific small-molecule tyrosine kinase inhibitors that competitively block the binding of adenosine triphosphate to its binding site in the tyrosine kinase domain of EGFR, thereby inhibiting autophosphorylation and blocking downstream signaling (Cataldo V D, N Engl J Med, 2011, 364, 947).

Second-Generation Inhibitors

Afatinib is an oral tyrosine kinase inhibitor (TKI) approved for the first-line treatment of patients with NSCLC whose tumors are driven by activating mutations of genes coding for epidermal growth factor receptor (EGFR). Afatinib is also an inhibitor of a specific EGFR mutation (T790M) that causes resistance to first-generation EGFR-targeted TKIs in about half of patients receiving those drugs. (Engle J A, Am J Health Syst Pharm 2014, 71 (22), 1933).

Neratinib, a pan-HER inhibitor, irreversible tyrosine kinase inhibitor binds and inhibits the tyrosine kinase activity of epidermal growth factor receptors, EGFR (or HER1), HER2 and HER4, which leads to reduced phosphorylation and activation of downstream signaling pathways. Neratinib has been shown to be effective against HER2-overexpressing or mutant tumors in vitro and in vivo. Neratinib is currently being investigated in various clinical trials in breast cancers and other solid tumors, including those with HER2 mutation (Feldinger K, Breast Cancer (Dove Med Press), 2015, 7, 147).

Dacomitinib is an irreversible inhibitor of EGFR, HER2, and HER4. In preclinical cell lines and xenograft studies, dacomitinib demonstrated activities against both activating EGFR mutations and EGFR T790M (Liao B C, Curr Opin Oncol. 2015, 27(2), 94).

Third-Generation Inhibitors

The third-generation EGFR-TKIs were designed to inhibit EGFR T790M while sparing wild-type EGFR.

AZD9291 (AstraZeneca, Macclesfield, UK), a mono-anilino-pyrimidine compound, is an irreversible mutant selective EGFR-TKI. This drug is structurally different from the first and second-generation EGFR-TKIs. In preclinical studies, it potently inhibited phosphorylation of EGFR in cell lines with activating EGFR mutations (EGFR del19 and EGFR L858R) and EGFR T790M. AZD9291 also caused profound and sustained tumor regression in tumor xenograft and transgenic mouse models harboring activating EGFR mutations and EGFR T790M. AZD9291 was less potent in inhibiting phosphorylation of wild-type EGFR cell lines (Liao B C, Curr Opin Oncol. 2015, 27(2), 94).

Rociletinib (CO-1686) (Clovis Oncology, Boulder, Colo), a 2,4-disubstituted pyrimidine molecule, is an irreversible mutant selective EGFR-TKI. In preclinical studies, CO-1686 led to tumor regression in cell-lines, xenograft models, and transgenic mouse models harboring activating EGFR mutations and EGFR T790M (Walter A O, Cancer Discov, 2013, 3(12), 1404).

HM61713 (Hanmi Pharmaceutical Company Ltd, Seoul, South Korea) is an orally administered, selective inhibitor for activating EGFR mutations and EGFR T790M. It has low activity against wild-type EGFR (Steuer C E, Cancer. 2015, 121(8), El).

Hillig et al 2019 PNAS describe compounds like

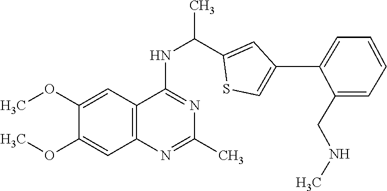 [SMILES: CNCc1ccccc1-c1csc(C(C)Nc2nc(C)nc3cc(OC)c(OC)cc23)c1]

as a potent SOS1 inhibitor and as a tool compound for further investigation of RAS-SOS1 biology in vitro.

WO2018/172250 (Bayer Pharma AG) describes 2-methyl-quinazoline like

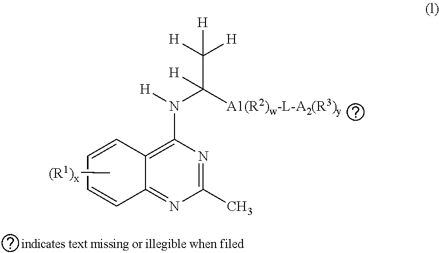

as inhibiting Ras-Sos interaction.

WO 2018/115380 (Boehringer Ingelheim) describes benzylamino substituted quinazolines like

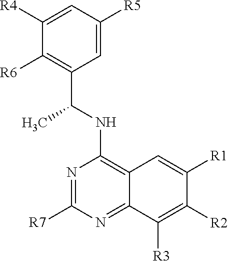

as SOS1 inhibitors.

WO2019/122129 (Boehringer Ingelheim) describes benzylaminosubstituted pyridopyrimidinoes like

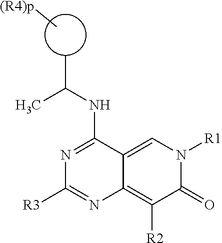

as SOS1 inhibitors.

It has now been found, and this constitutes the basis of the present invention, that the compounds of the present invention have surprising and advantageous properties.

In particular, the compounds of the present invention have surprisingly been found to effectively and selectively inhibit the Ras-Sos1 interaction without significantly targeting the EGFR receptor and may therefore be used for the treatment or prophylaxis of hyper-proliferative disorders, in particular cancer.

Furthermore the compounds of the present invention show good metablic stability and permeability.

### DESCRIPTION OF THE INVENTION

In accordance with a first aspect, the present invention covers compounds of general formula (I):

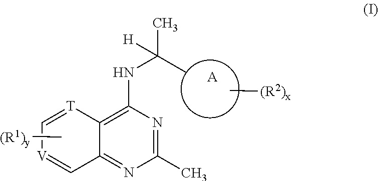

wherein
R¹ is selected from
   —H, halogen, —OH, —CN, —NO₂, C₁-C₆-alkylsulfanyl,
   —NR^{a}R^{b}, wherein R^{a} and R^{b} are independently selected from —H or C₁-C₆-alkyl,
   C₁-C₆-alkyl, C₁-C₆-alkoxy, C₂-C₆-alkenyl, C₂-C₆-alkynyl, C₃-C₈-cycloalkyl,
   C₄-C₈-cycloalkenyl, 4- to 7-membered heterocycloalkyl, 5- to 10 membered heterocycloalkenyl, heterospirocycloalkyl, fused heterocycloalkyl, bridged heterocycloalkyl, phenyl, heteroaryl, C₁-C₆-haloalkyl, —C(═O)OH,
   —C(═O)OR^{c}, wherein R^{c} stands for C₁-C₆-alkyl, C₃-C₆-alkenyl, C₃-C₆-alkynyl, C₃-C₈-cycloalkyl or C₄-C₈-cycloalkenyl,
   —N═S(═O)(R^{d})R^{e}, wherein R^{d} and R^{e} are independently selected from C₁-C₆-alkyl, C₂-C₆-alkenyl, C₂-C₆-alkynyl, C₃-C₈-cycloalkyl or C₄-C₈-cycloalkenyl,
   —NH—C(O)—C₁-C₆-alkyl,
   —NH—C(O)—NR^{a}R^{b}, wherein R^{a} and R^{b} are selected independently from a hydrogen atom or a C₁-C₆-alkyl,
   —NH—(CH₂)ₖ—NH—C(O)—C₁-C₆-alkyl, wherein k is 1 or 2,
   —NH—(CH₂)_{I}—R^{f}, wherein I is 0, 1 or 2 and R^{f} stands for a 4- to 7-membered heterocycloalkyl, heteroaryl or C₁-C₆-alkylsulfonyl,
      whereby in all foregoing definitions the C₁-C₆-alkyl-, C₁-C₆-alkoxy-, the 4- to 7-membered heterocycloalkyl and the heteroaryl can be optionally substituted, one or two or three times, identically or differently, with a halogen atom, hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy, C₁-C₆-alkylsulfonyl, phenyl, benzyl, heteroaryl, —CH₂-heteroaryl, C₃-C₈-cycloalkoxy. phenyloxy, heteroaryloxy, —NH—C(O)—C₁-C₆-alkyl or —NR^{a}R^{b}, wherein R^{a} and R^{b} are independently selected from a hydrogen atom or C₁-C₆-alkyl,
   —O—(CH₂)_{z}-phenyl, —O(CH₂)_{z}-C₄-C₇-heterocycloalkyl, —O(CH₂)_{z}-heteroaryl, wherein z is 0, 1 or 2, and the phenyl, heterocycloalkyl and heteroaryl can optionally be substituted with a group selected from hydroxy, heterocycloalkyl or heterocyclyoalkenyl, which both can be substituted with a methyl- and/or oxo-group,

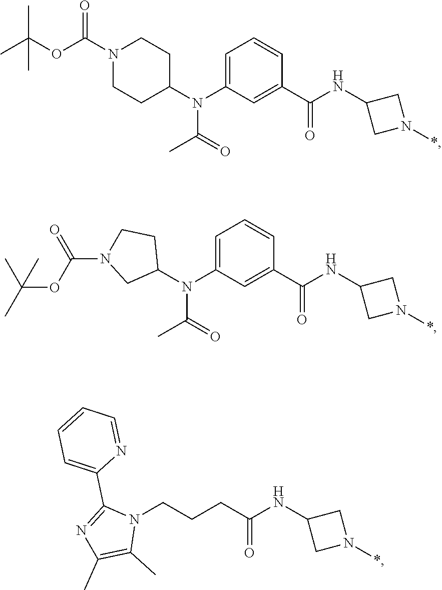

wherein L₂a stands for C(O), L₂b stands for a bond or C₁-C₆-alkylene, alkylene, X2 stands for

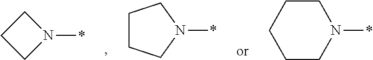 [SMILES: *N1CCC1 ; *N1CCCC1 ; *N1CCCCC1]

and Rx₂ stands for

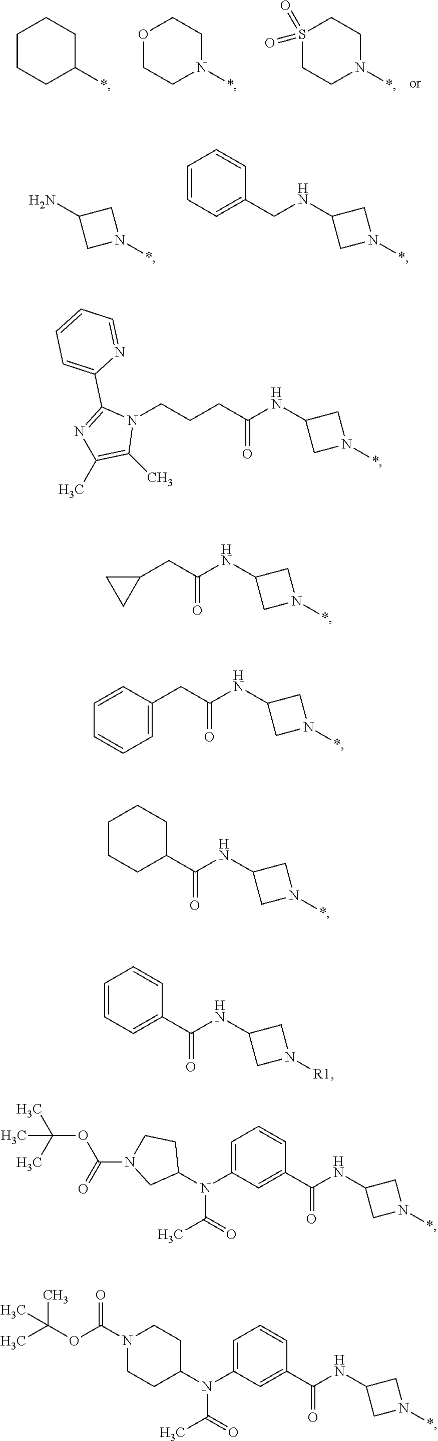

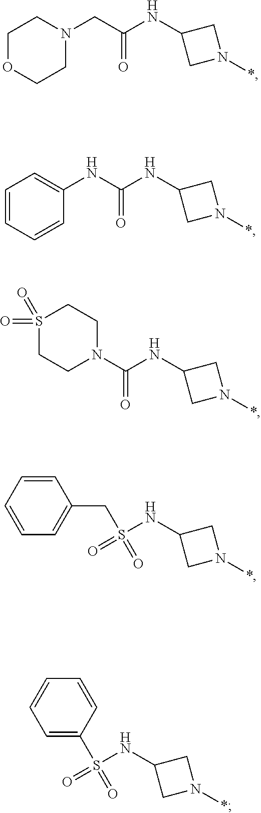

or in which a further R¹ as defined above can be directly attached to a first R¹ equaling C₁-C₆-alkyl, C₁-C₆-alkoxy, C₂-C₆-alkenyl, C₂-C₆-alkynyl, C₃-C₈-cycloalkyl, C₄-C₈-cycloalkenyl, 4- to 7-membered heterocycloalkyl, 5- to 10 membered heterocycloalkenyl, heterospirocycloalkyl, fused heterocycloalkyl, bridged heterocycloalkyl, phenyl, heteroaryl, C₁-C₆-haloalkyl,
y is 1, 2 or 3;
and either both T and V stand for nitrogen or T stands for carbon and V for nitrogen or T for nitrogen and V for carbon;
A is selected from the group consisting of C₆₋₁₀aryl, 5-10 membered heteroaryl and 9-10 membered bicyclic heterocyclyl;
R² is each independently selected from the group consisting of C₁₋₄alkyl, C₂₋₄alkenyl, C₂₋₄alkinyl, C₁₋₄haloalkyl, hydroxy-C₁₋₄alkyl, hydroxy-C₁₋₄haloalkyl, C₃₋₆cycloalkyl, 3-6 membered heterocyclyl, hydroxy-C₃₋₆cycloalkyl, C₁₋₄haloalkyl substituted with a 3-6 membered heterocyclyl, 3-6 membered heterocyclyl substituted with hydroxy, halogen, —NH₂, —SO₂—C₁₋₄alkyl and the bivalent substituent ═O, while ═O may only be a substituent in a non-aromatic ring;
x is 1, 2 or 3;
or a tautomer, an N-oxide, a hydrate, a solvate, or a salt thereof, or a mixture of same.

In accordance with a further first aspect, the present invention covers compounds of general formula (Ia):

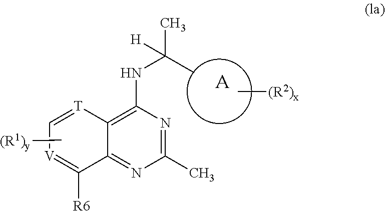

wherein
R¹ is selected from
   —H, halogen, —OH, —CN, —NO₂, C₁-C₆-alkylsulfanyl,
   —NR^{a}R^{b}, wherein R^{a} and R^{b} are independently selected from —H or C₁-C₆-alkyl,
   C₁-C₆-alkyl, C₁-C₆-alkoxy, C₂-C₆-alkenyl, C₂-C₆-alkynyl, C₃-C₈-cycloalkyl,
   C₄-C₈-cycloalkenyl, 4- to 7-membered heterocycloalkyl, 5- to 10 membered heterocycloalkenyl, heterospirocycloalkyl optionally substituted by an oxo-group (═O), fused heterocycloalkyl optionally substituted by an oxo-group (═O), bridged heterocycloalkyl optionally substituted by an oxo-group (═O), phenyl, heteroaryl, C₁-C₆-haloalkyl, —C(═O)OH,
   —C(═O)OR^{c}, wherein R^{c} stands for C₁-C₆-alkyl, C₃-C₆-alkenyl, C₃-C₆-alkynyl, C₃-C₈-cycloalkyl or C₄-C₈-cycloalkenyl,
   —N═S(═O)(R^{d})R^{e}, wherein R^{d} and R^{e} are independently selected from C₁-C₆-alkyl, C₂-C₆-alkenyl, C₂-C₆-alkynyl, C₃-C₈-cycloalkyl or C₄-C₈-cycloalkenyl,
   —NH—C(O)—C₁-C₆-alkyl,
   —NH—C(O)—NR^{a}R^{b}, wherein R^{a} and R^{b} are selected independently from a hydrogen atom or a C₁-C₆-alkyl,
   —NH—(CH₂)ₖ—NH—C(O)—C₁-C₆-alkyl, wherein k is 1 or 2,
   —NH—(CH₂)_{I}-R^{f}, wherein I is 0, 1 or 2 and R^{f} stands for a 4- to 7-membered heterocycloalkyl, heteroaryl or C₁-C₆-alkylsulfonyl,
      whereby in all foregoing definitions the C₁-C₆-alkyl-, C₁-C₆-alkoxy-, the 4- to 7-membered heterocycloalkyl and the heteroaryl can be optionally substituted, one or two or three times, identically or differently, with a halogen atom, hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy, C₁-C₆-alkylsulfonyl, phenyl, benzyl, heteroaryl, —CH₂-heteroaryl, C₃-C₈-cycloalkoxy. phenyloxy, heteroaryloxy, —NH—C(O)—C₁-C₆-alkyl or —NR^{a}R^{b}, wherein R^{a} and R^{b} are independently selected from a hydrogen atom or C₁-C₆-alkyl,
   —O—(CH₂)_{z}-phenyl, —O(CH₂)_{z}-C₄-C₇-heterocycloalkyl, —O(CH₂)_{z}-heteroaryl, wherein z is 0, 1 or 2, and the phenyl, heterocycloalkyl and heteroaryl can optionally be substituted with a group selected from hydroxy, heterocycloalkyl or heterocaclyoalkenyl, which both can be substituted with a methyl- and/or oxo-group,

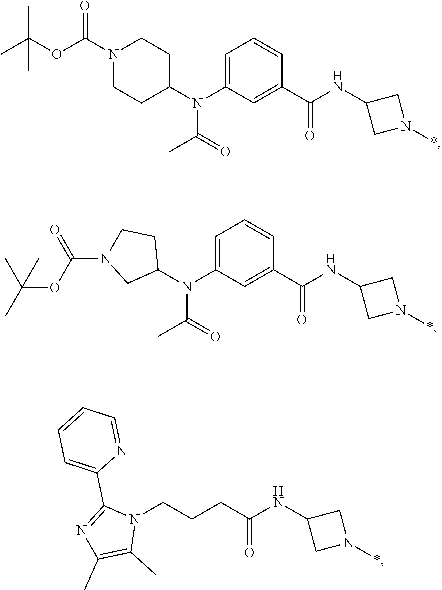

wherein L₂a stands for C(O), L₂b stands for a bond or C₁-C6-alkylene, alkylene, X2 stands for

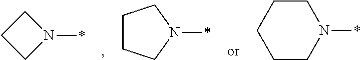

and Rx₂ stands for

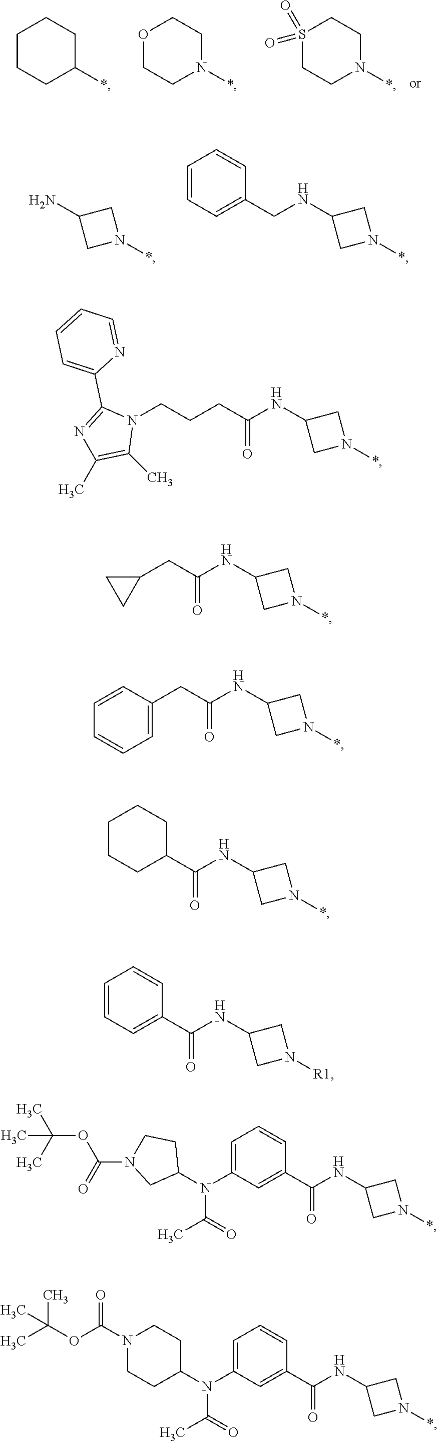

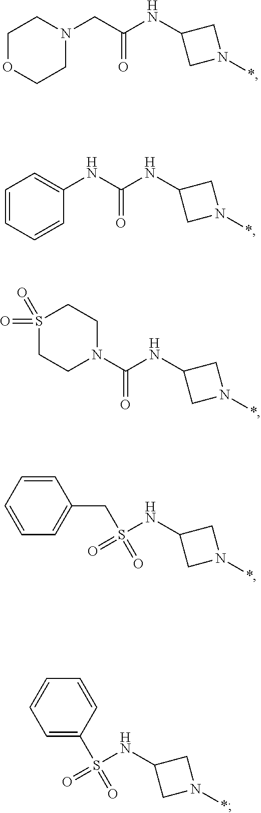

or in which a further R¹ as defined above can be directly attached to a first R¹ equaling C₁-C₆-alkyl, C₁-C₆-alkoxy, C₂-C₆-alkenyl, C₂-C₆-alkynyl, C₃-C₈-cycloalkyl, C₄-C₈-cycloalkenyl, 4- to 7-membered heterocycloalkyl, 5- to 10 membered heterocycloalkenyl, heterospirocycloalkyl, fused heterocycloalkyl, bridged heterocycloalkyl, phenyl, heteroaryl, C₁-C₆-haloalkyl,
y is 1, 2 or 3;
and either both T and V stand for nitrogen or T stands for carbon and V for nitrogen or T for nitrogen and V for carbon;
A is selected from the group consisting of C₆₋₁₀aryl, 5-10 membered heteroaryl and 9-10 membered bicyclic heterocyclyl;
R² is each independently selected from the group consisting of C₁₋₄alkyl, C₂₋₄alkenyl, C₂₋₄alkinyl, C₁₋₄haloalkyl, hydroxy-C₁₋₄alkyl, hydroxy-C₁₋₄haloalkyl, C₃₋₆cycloalkyl, 3-6 membered heterocyclyl, hydroxy-C₃₋₆cycloalkyl, C₁₋₄haloalkyl substituted with a 3-6 membered heterocyclyl, 3-6 membered heterocyclyl substituted with hydroxy, halogen, —NH₂, —SO₂—C₁₋₄alkyl and the bivalent substituent ═O, while ═O may only be a substituent in a non-aromatic ring;
R⁶ is selected from the group consisting of —H, halogen, C₁₋₄alkyl, C₃₋₇-cycloalkyl, C₄₋₇heterocycloalkyl optionally comprising 1 or 2 nitrogen, 1 oxygen or 1 sulphur atom, —O—C₁₋₄alkyl, —NH₂, —NH(C₁₋₄alykl) or —NH(C₁₋₄alkyl)₂,
x is 1, 2 or 3;
or a tautomer, an N-oxide, a hydrate, a solvate, or a salt thereof, or a mixture of same.

Alternatively R⁶ of formula (Ia) is selected from the group consisting of —H, —CH₃, —CH(CH₃)₂, —CH₂OH, —CF₃ or —CHF₂.

Definitions

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one parts, e.g. (C₁-C₄-alkoxy)-(C₁-C₄-alkyl) it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methyl butyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-hydroxyalkyl” means a linear or branched, saturated, monovalent hydrocarbon group in which the term “C₁-C₆-alkyl” is defined supra, and in which 1, 2 or 3 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-alkylsulfanyl” means a linear or branched, saturated, monovalent group of formula (C₁-C₆-alkyl)-S-, in which the term “C₁-C₆-alkyl” is as defined supra, e.g. a methylsulfanyl, ethylsulfanyl, propylsulfanyl, isopropylsulfanyl, butylsulfanyl, sec-butylsulfanyl, isobutylsulfanyl, tert-butylsulfanyl, pentylsulfanyl, isopentylsulfanyl, hexylsulfanyl group.

The term “C₁-C₆-alkylsulfonyl” means a linear or branched, saturated, monovalent group of formula (C₁-C₆-alkyl)-SO₂—, in which the term “C₁-C₆-alkyl” is as defined supra, e.g. a methylsulfonyl, ethylsulfonyl, propylsulfonyl, isopropylsulfonyl, butylsulfonyl, sec-butylsulfonyl, isobutylsulfonyl, tert-butylsulfonyl, pentylsulfonyl, isopentylsulfonyl, hexylsulfonyl group.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₂-C₆-alkenyl” means a linear or branched, monovalent hydrocarbon group, which contains one or two double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then it is possible for said double bonds to be isolated from, or conjugated with, each other. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group. Particularly, said group is vinyl or allyl.

The term “C₂-C₆-alkynyl” means a linear or branched, monovalent hydrocarbon group which contains one triple bond, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group. Particularly, said alkynyl group is ethynyl, prop-1-ynyl or prop-2-ynyl.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, or a bicyclic hydrocarbon ring, e.g. a bicyclo[4.2.0]octyl or octahydropentalenyl.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g. a cyclobutenyl, cyclopentenyl, cyclohexenyl, cycloheptenyl or cyclooctenyl group, or a bicyclic hydrocarbon ring, e.g. a bicyclo[2.2.1]hept-2-enyl or bicyclo[2.2.2]oct-2-enyl.

The term “C₃-C₈-cycloalkoxy” means a saturated, monovalent, mono- or bicyclic group of formula (C₃-C₈-cycloalkyl)-O—, which contains 3, 4, 5, 6, 7 or 8 carbon atoms, in which the term “C₃-C₈-cycloalkyl” is defined supra, e.g. a cyclopropyloxy, cyclobutyloxy, cyclopentyloxy, cyclohexyloxy, cycloheptyloxy or cyclooctyloxy group.

The term “spirocycloalkyl” means a saturated, monovalent bicyclic hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom.

Said spirocycloalkyl group is, for example, spiro[2.2]pentyl, spiro[2.3]hexyl, spiro[2.4]heptyl, spiro[2.5]octyl, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The terms “4- to 7-membered heterocycloalkyl” means a monocyclic, saturated heterocycle with 4, 5, 6 or 7 ring atoms in total, which contains one or two identical or different ring heteroatoms from the series N, O and S, it being possible for said heterocycloalkyl group to be attached to the rest of the molecule via any one of the carbon atoms or, if present, a nitrogen atom.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen atom and optionally one further ring heteroatom from the series: N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O.

The term “4- to 7-memebered azacycloalkyl” means a monocyclic saturated heterocycly with 4, 5, 6 or 7 ring atoms in total which is attached to the rest of the molecule via the nitrogen atom and which optionally contains one more heteroatom selected from nitrogen and oxygen.

Said 4- to 7-membered azacycloalkyl group, without being limited thereto, can be a 4-membered ring, such as azetidin-1-yl, for example; or a 5-membered ring, such as pyrrolidin-1-yl, imidazolidin-1-yl, pyrazolidin-1-yl, 1,2-oxazolidin-2-yl or 1,3-oxazolidin-3-yl, for example; or a 6-membered ring, such as piperidin-1-yl, morpholin-4-yl, piperazin-1-yl or 1,2-oxazinan-2-yl, for example, or a 7-membered ring, such as azepan-1-yl, 1,4-diazepan-1-yl or 1,4-oxazepan-4-yl, for example.

The term “5- to 10-membered heterocycloalkenyl” means a monocyclic, unsaturated, non-aromatic heterocycle with 5, 6, 7, 8, 9 or 10 ring atoms in total, which contains one or two double bonds and one or two identical or different ring heteroatoms from the series: N, O, S; it being possible for said heterocycloalkenyl group to be attached to the rest of the molecule via any one of the carbon atoms or, if present, a nitrogen atom.

Said heterocycloalkenyl group is, for example, 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl or 4H-[1,4]thiazinyl.

The term “heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one, two or three identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro [5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “6- to 10-membered azaspirocycloalkyl” means a bicyclic, saturated heterocycle with 6, 7, 8, 9 or 10 ring atoms in total, in which the two rings share one common ring carbon atom and which is bound to the rest of the molecule via the nitrogen atom and which azaspirocycloalkyl may contain up to 2 further heteroatoms selected from nitrogen and oxygen.

Said azaspirocycloalkyl is for example, azaspiro[2.3]hexyl, azaspiro[3.3]heptyl, oxaazaspiro[3.3]heptyl, oxazaspiro[5.3]nonyl, oxazaspiro[4.3]octyl, azaspiro[4,5]decyl, oxazaspiro[5.5]undecyl, diazaspiro[3.3]heptyl, triazaspiro[3.4]octyl or one of the further homologous scaffolds such as spiro[3.4]-, spiro[4.4]-, spiro[2.4]-, spiro[2.5]-, spiro[2.6]-, spiro[3.5]-, spiro[3.6]- and spiro[4.5]-, whereby these azaspirocycloalkyl groups are always bound via the nitrogen atom to the rest of the molecule.

Of these groups preference is given to 2-oxa-6-azaspiro[3.3]hept-6-yl and 2,5,7-triazaspiro[3.4]octan-2-yl.

The term “fused heterocycloalkyl” means a bicyclic, saturated heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl.

The term “bridged heterocycloalkyl” means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo-[2.2.2]octyl, diazabicyclo[2.2.2]octyl, oxazabicyclo[2.2.2]octyl, thiazabicyclo[2.2.2]octyl, azabi-cyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, 0 and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; a 8-membered heteroaryl group, such as for example 6,7-dihydro-5H-pyrrolo[1,2-a]imidazolyl or a 9-membered heteroaryl group, such as, for example, benzofuranyl, benzothienyl, benzoxazolyl, benzisoxazolyl, benzimidazolyl, benzothiazolyl, benzothiadiazolyl, benzotriazolyl, indazolyl, indolyl, isoindolyl, indolizinyl, thienopyridinyl, 1H-pyrrolo[2,3-b]pyridinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

A C4 to C12 carbocyclic, heterocyclic, optionally bicyclic, optionally aromatic or optionally heteroaromatic ring system, wherein in a bicyclic, aromatic or heteroaromatic ring system one or two double bonds can be hydrogenated is selected from the group of the substituents phenyl, naphthyl, 1,2,3,4-tetrahydronaphthyl, 1,3-benzodioxolyl, quinolinyl, isoquinolinyl, 2,3-dihydro-1,4-benzodioxinyl, imidazo[1,2-a]pyridinyl, furanyl, thienyl, pyridinyl, 2H-1,4-benzoxazinyl-3(4H)-one, 2,1,3-benzothiadiazolyl, 1-benzofuranyl, 1-benzothienyl, 1H-indazolyl, 1H-indolyl, 1H-benzimidazolyl, 1,3-benzothiazolyl, thieno[2,3-b]pyridinyl, thieno[2,3-c]pyridinyl, thieno[3,2-c]pyridinyl, pyrimidinyl, 1H-pyrazolyl, 6,7-dihydro-5H-pyrrolo[1,2-a]imidazolyl, 1,2-oxazolyl, 1H-imidazolyl, 1,3,4-oxadiazolyl, 1H-tetrazolyl, 1H-pyrrolyl, 1H-pyrrolo[2,3-b]pyridinyl or 3,4-dihydro-2H-1,4-benzoxazinyl.

Particularly, the heteroaryl group is a quinolinyl, isoquinolinyl, imidazo[1,2-a]pyridinyl, furanyl, thienyl, pyridinyl, 2,1,3-benzothiadiazolyl, 1-benzofuranyl, 1-benzothiophenyl, 1H-indazolyl, 1H-indolyl, 1H-benzimidazolyl, 1,3-benzothiazolyl, thieno[2,3-b]pyridinyl, thieno[2,3-c]pyridinyl, thieno[3,2-c]pyridinyl, pyrimidinyl, 1H-pyrazolyl, 6,7-dihydro-5H-pyrrolo[1,2-a]imidazolyl, 1,2-oxazolyl, 1H-imidazolyl, 1,3,4-oxadiazolyl, 1H-tetrazolyl, 1H-pyrrolyl, 1H-pyrrolo[2,3-b]pyridinyl or 3,4-dihydro-2H-1,4-benzoxazinyl group.

In composite substituents such as C₁-C₆-haloalkyl, C₁-C₄-haloalkyl, C₁-C₆-haloalkoxy, —(CH₂)-heteroaryl, heteroaryloxy, —O—(CH₂)ₓ-heteroaryl, —O—(CH₂)_{z}-heteroaryl, O—(CH₂)-4- to 7-membered heterocycloalkyl, bicyclic heteroaryl, C₁-C₆-hydroxyalkyl, —O—(CH₂)ₓ—C₃-C₈-cycloalkyl, O—(CH₂)ₓ-phenyl, —O—(CH₂)ₓ-heterocyclyl and C₃-C₈-cycloalkyloxy the definition of the residue to which the further substituent is attached is the same as given for the residues which do not bear a further substituent, e.g. in C₁-C₆-haloalkyl the C₁-C₆-alkyl has the same meanings as given for the C₁-C₆-alkyl earlier.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:

“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;

“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;

“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;

“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;

“C₃-C₆” encompasses C₃, C₄, C₅, C₆, C₃-C₆, C₃-C₅, C₃-C₄, C₄-C₆, C₄-C₅, and C₅-C₆;

“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;

“C₄-C₇” encompasses C₄, C₅, C₆, C₇, C₄-C₇, C₄-C₆, C₄-C₅, C₅-C₇, C₅-C₆ and C₆-C₇;

“C₄-C₆” encompasses C₄, C₅, C₆, C₄-C₆, C₄-C₅ and C₅-C₆;

“C₅-C₁₀” encompasses C₅, C₆, C₇, C₈, C₉, C₁₀, C₅-C₁₀, C₅-C₉, C₅-C₈, C₅-C₇, C₅-C₆, C₆- C₁₀, C₆-C₉, C₆-C₈, C₆-C₇, C₇-C₁₀, C₇-C₉, C₇-C₈, C₈-C₁₀, C₈-C₉ and C₉-C₁₀;

“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

In another embodiment the present invention concerns a deuterium-containing compound of general formula (I), in which one, two or three of the hydrogen atom(s) in either one or both of the methyl groups shown in general formula (I) is/are replaced with a deuterium atom.

Also the hydrogen atom on the carbon atom between the nitrogen atom and the group A1 can be replaced with a deuterium atom either as the single replacement of a hydrogen by a deuterium or in addition to the beforementioned replacements in either one or both of the methyl groups shown in general formula (I).

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention contain at least one or optionally even more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred isomers are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel O D and Chiracel O J, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

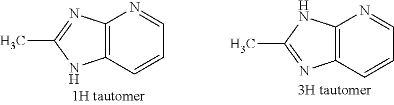

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

As used herein, the term “in vivo hydrolysable ester” means an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₈ alkoxymethyl esters, e.g. methoxymethyl, C₁-C₈ alkanoyloxymethyl esters, e.g. pivaloyloxymethyl, phthalidyl esters, C₃-C₈ cycloalkoxy-carbonyloxy-C₁-C₈ alkyl esters, e.g. 1-cyclohexylcarbonyloxyethyl; 1,3-d ioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, it being possible for said esters to be formed at any carboxy group in the compounds of the present invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In accordance with other embodiments, the present invention covers the following compounds.

A) A compound of formula I or Ia, wherein
R¹ is selected from
   —H, —Br, —OH, —NO₂, —CH₃,

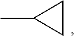

—O—CH₃, —O—CH₂—CH₃, —O—CH(CH₃)₂, —O—(CH₂)₃CH₃, —O—(CH₂)₂CH(CH₃)₂,

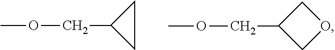

—O —CH₂-phenyl, —O—(CH₂)₂—O—CH₃, —O—(CH₂)₂—S(O)₂—CH₃, —CH₂—OH, —C(CH₃)₂—OH, —C(O)OH, —C(O)OCH₃, —NH₂, —NH(CH₃), —N(CH₃)₂,

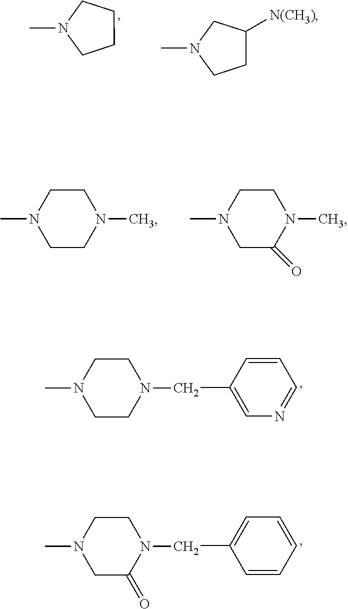

—NH—(CH₂)₂—NH—C(O)—CH₃, —NH—(CH₂)₂-morpholino, —NH—C(O)—CH₃, —NH—C(O)—NH—CH₃, —NH—C(O)—N(CH₃)₂, —NH—S(O)₂—CH₃, —N═S(O)(CH₃)₂,

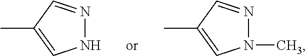

y is 1 or 2;
A is selected from the group consisting of C₆₋₁₀aryl, 5-10 membered heteroaryl and 9-10 membered bicyclic heterocyclyl;
R² is each independently selected from the group consisting of C₁₋₄alkyl, C₂₋₄alkenyl, C₂₋₄alkinyl, C₁₋₄haloalkyl, hydroxy-C₁₋₄alkyl, hydroxy-C₁₋₄haloalkyl, C₃₋₆cycloalkyl, 3-6 membered heterocyclyl, hydroxy-C₃₋₆cycloalkyl, C₁₋₄haloalkyl substituted with a 3-6 membered heterocyclyl, 3-6 membered heterocyclyl substituted with hydroxy, halogen, —NH₂, —SO₂-C₁₋₄alkyl and the bivalent substituent ═O, while ═O may only be a substituent in a non-aromatic ring
x is 1, 2 or 3;
or a tautomer, an N-oxide, a hydrate, a solvate, or a salt thereof, or a mixture of same.

B) A compound as defined in A (above), wherein
A is selected from the group consisting of C₆₋₁₀-aryl, 5-10 membered heteroaryl and 9-10 membered bicyclic heterocyclyl;
R2 is each independently selected from the group consisting of C₁₋₄-alkyl, C₂₋₄-alkenyl, C₂₋₄-alkinyl, C₁₋₄-haloalkyl, hydroxy-C₁₋₄-alkyl, hydroxy-C₁₋₄-haloalkyl, C₃₋₆-cycloalkyl, 3-6 membered heterocyclyl, hydroxy-C₃₋₆-cycloalkyl, C₁₋₄-haloalkyl substituted with a 3-6 membered heterocyclyl, 3-6 membered heterocyclyl substituted with hydroxy, halogen, —NH₂, —SO₂-C₁₋₄-alkyl and the bivalent substituent ═O, while ═O may only be a substituent in a non-aromatic ring
x is 1, 2 or 3
or a tautomer, an N-oxide, a hydrate, a solvate, or a salt thereof, or a mixture of same.

C) A compound as defined in A or B (above), wherein
A is selected from the group consisting of C₆₋₁₀aryl, 5-10 membered heteroaryl and 9-10 membered bicyclic heterocyclyl;
x is 1 or 2
R² is each independently selected from the group consisting of C₁₋₄-alkyl, C₂₋₄-alkinyl, C₁₋₄-haloalkyl, hydroxy-C₁₋₄-haloalkyl, C₁₋₄-haloalkyl substituted with a 3-6 membered heterocyclyl, halogen, and the bivalent substituent ═O, while ═O may only be a substituent in a non-aromatic ring
or a tautomer, an N-oxide, a hydrate, a solvate, or a salt thereof, or a mixture of same.

D) A compound as defined in A, B or C (above), wherein

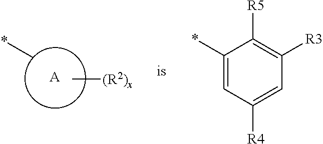

and wherein
R³ is selected from the group consisting of C₁₋₄-alkyl, C₁₋₄-haloalkyl, hydroxy-C₁₋₄-alkyl, hydroxy-C₁₋₄-haloalkyl, C₁₋₄-haloalkyl substituted with a 3-6 membered heterocyclyl, C₃₋₆-cycloalkyl, hydroxy-C₃₋₆-cycloalkyl, 3-6 membered heterocyclyl, 3-6 membered hydroxy-heterocyclyl, halogen and —SO₂—C₁₋₄-alkyl;
R⁴ is selected from the group consisting of hydrogen and —NH₂,
R⁵ is selected from the group consisting of hydrogen, C₁₄-alkyl and halogen;
or
R³ and R⁵ together with the carbon atoms they are attached form a 5-6 membered nonaromatic carbocycle, a 5-6 membered non-aromatic heterocycle or a 5-6 membered heteroaryl, wherein the 5-6 membered non-aromatic carbocycle, 5-6 membered nonaromatic heterocycle and 5-6 membered heteroaryl are all optionally substituted by one or more halogen or by an oxo group
or a tautomer, an N-oxide, a hydrate, a solvate, or a salt thereof, or a mixture of same.

E) A compound as defined in A, B, C or D (above), wherein
R³ is selected from the group consisting of C₁₋₄-haloalkyl, hydroxy-C₁₋₄-haloalkyl and C₁₋₄-haloalkyl substituted with a 3-6 membered heterocyclyl;
R⁴ is hydrogen;
R⁵ is selected from the group consisting of hydrogen, C₁₋₄-alkyl and fluorine;
or
R³ and R⁵ together with the carbon atoms they are attached form a 5-6 membered nonaromatic carbocycle, a 5-6 membered non-aromatic heterocycle or a 5-6 membered heteroaryl, wherein the 5-6 membered non-aromatic carbocycle, 5-6 membered nonaromatic heterocycle and 5-6 membered heteroaryl are all optionally substituted by one or more fluorine or by an oxo group
or a tautomer, an N-oxide, a hydrate, a solvate, or a salt thereof, or a mixture of same.

F) A compound as defined in A, B, C, D or E (above), wherein

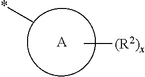

is selected from

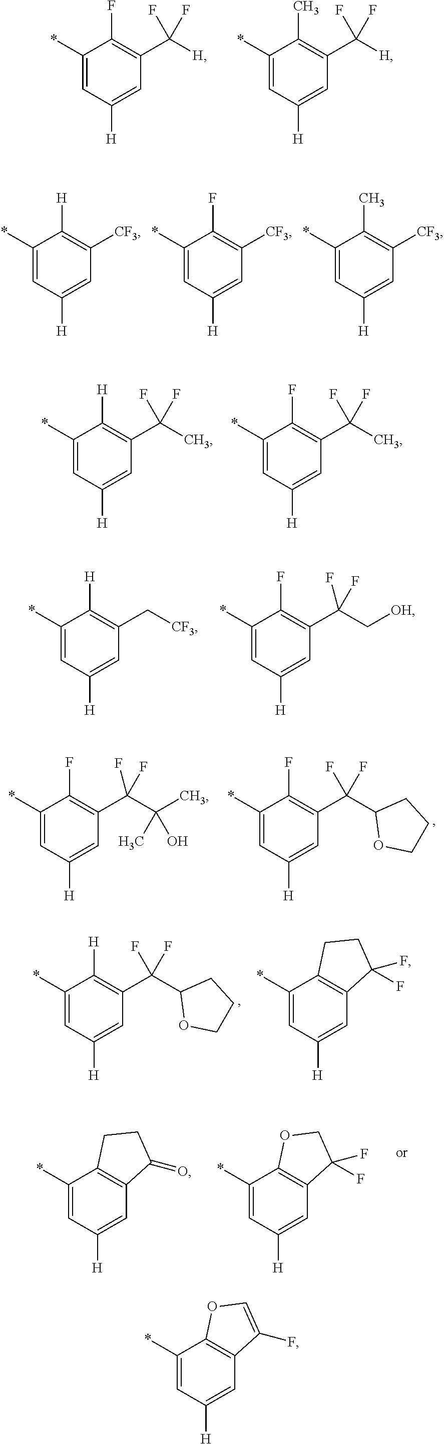

or a stereoisomer, a tautomer, an N-oxide, a hydrate, a solvate, or a salt thereof, or a mixture of same.

G) The compound as defined in A, B, C, D, E or F (above), wherein V is nitrogen and T is carbon or a stereoisomer, a tautomer, an N-oxide, a hydrate, a solvate, or a salt thereof, or a mixture of same.

H) The compound as defined in A, B, C, D, E, F or G (above), wherein y=1 and R¹ is selected from

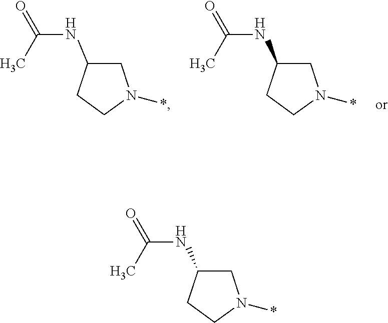

or a stereoisomer, a tautomer, an N-oxide, a hydrate, a solvate, or a salt thereof, or a mixture of same.

I) The compound as defined in A, B, C, D, E, F, G or H (above), wherein V is nitrogen, T is carbon, y=1,
R¹ is selected from

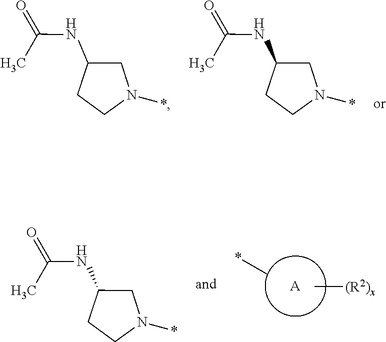

is selected from

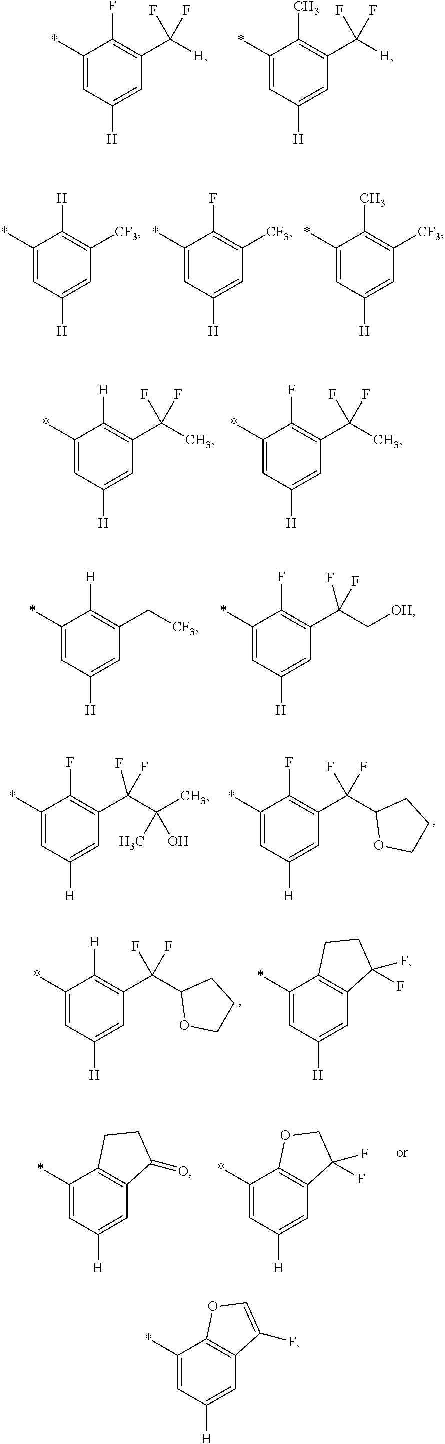

or a stereoisomer, a tautomer, an N-oxide, a hydrate, a solvate, or a salt thereof, or a mixture of same.

J) The compound as defined in A, B, C, D, E, F, G, H or I (above), which is selected from the group consisting of:
N-[(3R)-1-[2-methyl-4-[[(1R)-1-[3-(trifluoromethyl)phenyl]ethyl]amino]pyrido[3,4-d]pyrimidin-6-yl]pyrrolidin-3-yl]acetamide
N-[(3S)-1-[2-methyl-4-[[(1R)-1-[3-(trifluoromethyl)phenyl]ethyl]amino]pyrido[3,4-d]pyrimidin-6-yl]pyrrolidin-3-yl]acetamide
N-[(3R)-1-[4-[[(1R)-1-[3-(difluoromethyl)-2-fluoro-phenyl]ethyl]amino]-2-methyl-pyrido[3,4-d]pyrimidin-6-yl]pyrrolidin-3-yl]acetamide
N-[(3S)-1-[4-[[(1R)-1-[3-(difluoromethyl)-2-fluoro-phenyl]ethyl]amino]-2-methyl-pyrido[3,4-d]pyrimidin-6-yl]pyrrolidin-3-yl]acetamide
N-[(3R)-1-[4-[[(1R)-1-[3-(difluoromethyl)-2-methyl-phenyl]ethyl]amino]-2-methyl-pyrido[3,4-d]pyrimidin-6-yl]pyrrolidin-3-yl]acetamide
N-[(3S)-1-[4-[[(1R)-1-[3-(difluoromethyl)-2-methyl-phenyl]ethyl]amino]-2-methyl-pyrido[3,4-d]pyrimidin-6-yl]pyrrolidin-3-yl]acetamide
N-[(3R)-1-[4-[[(1R)-1-[3-(1,1-difluoroethyl)phenyl]ethyl]amino]-2-methyl-pyrido[3,4-d]pyrimidin-6-yl]pyrrolidin-3-yl]acetamide
N-[(3S)-1-[4-[[(1R)-1-[3-(1,1-difluoroethyl)phenyl]ethyl]amino]-2-methyl-pyrido[3,4-d]pyrimidin-6-yl]pyrrolidin-3-yl]acetamide
N-[(3R)-1-[4-[[(1R)-1-[3-(1,1-difluoroethyl)-2-fluoro-phenyl]ethyl]amino]-2-methyl-pyrido[3,4-d]pyrimidin-6-yl]pyrrolidin-3-yl]acetamide
N-[(3S)-1-[4-[[(1R)-1-[3-(1,1-difluoroethyl)-2-fluoro-phenyl]ethyl]amino]-2-methyl-pyrido[3,4-d]pyrimidin-6-yl]pyrrolidin-3-yl]acetamide
N-{(1R)-1-[3-(difluoromethyl)-2-fluorophenyl]ethyl}-6-fluoro-2,8-dimethylpyrido[3,4-d]pyrimidin-4-amine
N-{(3R)-1-[4-({(1R)-1-[3-(difluoromethyl)-2-fluorophenyl]ethyl}amino)-2,8-dimethylpyrido[3,4-d]pyrimidin-6-yl]pyrrolidin-3-yl}acetamide
or a stereoisomer, a tautomer, an N-oxide, a hydrate, a solvate, or a salt thereof, or a mixture of same.

K) A SOS1 inhibitor compound as described herein or as defined in A, B, C, D, E, F, G, H, I or J (above) for use in the treatment and/or prevention of cancer, wherein said SOS1 inhibitor compound is administered in combination with at least one other pharmacologically active substance and wherein each of said other pharmacologically active substance(s) is selected from the group consisting of: an inhibitor of HRas, NRas or KRAS and mutants thereof, in particular an inhibitor of KRAS-G12C; an inhibitor of MAP kinases, in particular MEK1, MEK2, ERK1, ERK2, ERKS and/or of an inhibitor of PI3-kinases and mutants thereof; an inhibitor of Tropomyosin Receptor kinases and/or of mutants thereof; an inhibitor of SHP2 and mutants thereof; inhibitor of EGFR and/or of mutants thereof; an inhibitor of FGFR1 and/or FGFR2 and/or FGFR3 and/or of mutants thereof; an inhibitor of ALK and/or of mutants thereof; an inhibitor of c-MET and/or of mutants thereof; an inhibitor of BCR-ABL and/or of mutants thereof; an inhibitor of ErbB2 (Her2) and/or of mutants thereof; an inhibitor of AXL and/or of mutants thereof; an inhibitor of A-Raf and/or B-Raf and/or C-Raf and/or of mutants thereof; an inhibitor of mTOR and mutants thereof; an inhibitor of IGF1/2 and/or of IGF1-R; an inhibitor of farnesyl transferase.

In accordance with further embodiments, the present invention covers the following compounds.

A compound of formula I or la as defined in A, B, C, D, E, F, G, H or I (above), wherein

R¹ is selected from

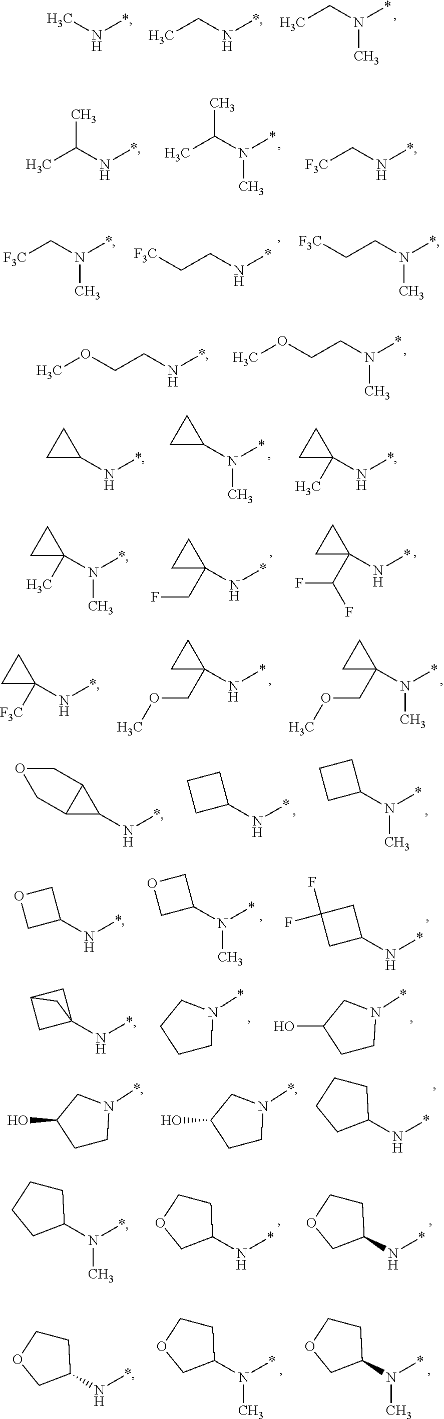

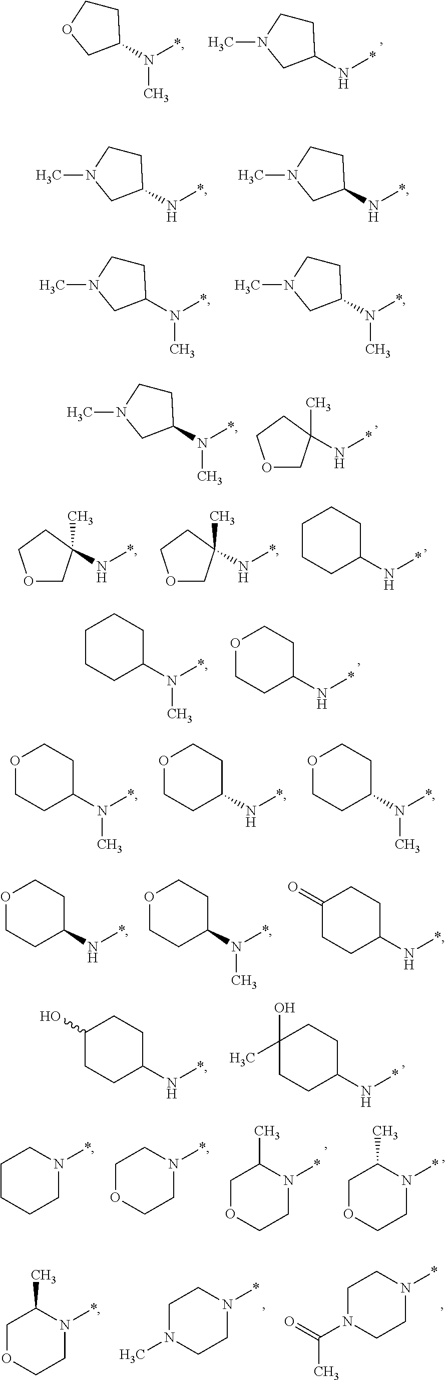

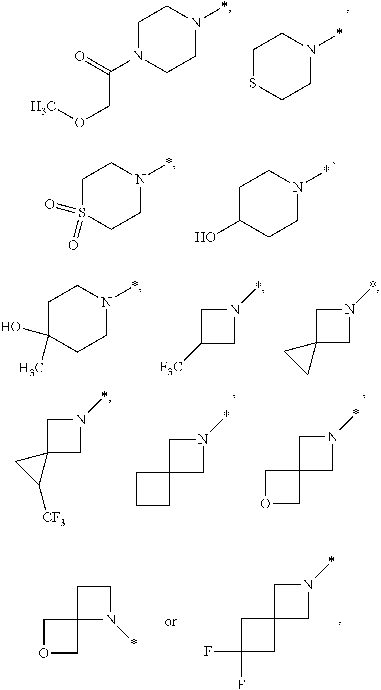

R¹ can also be selected from

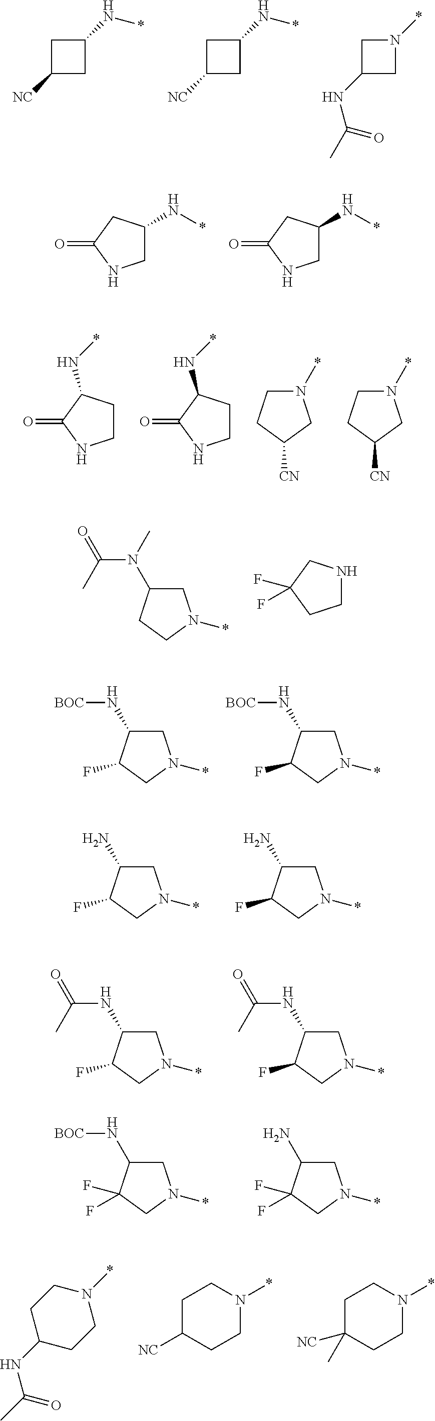

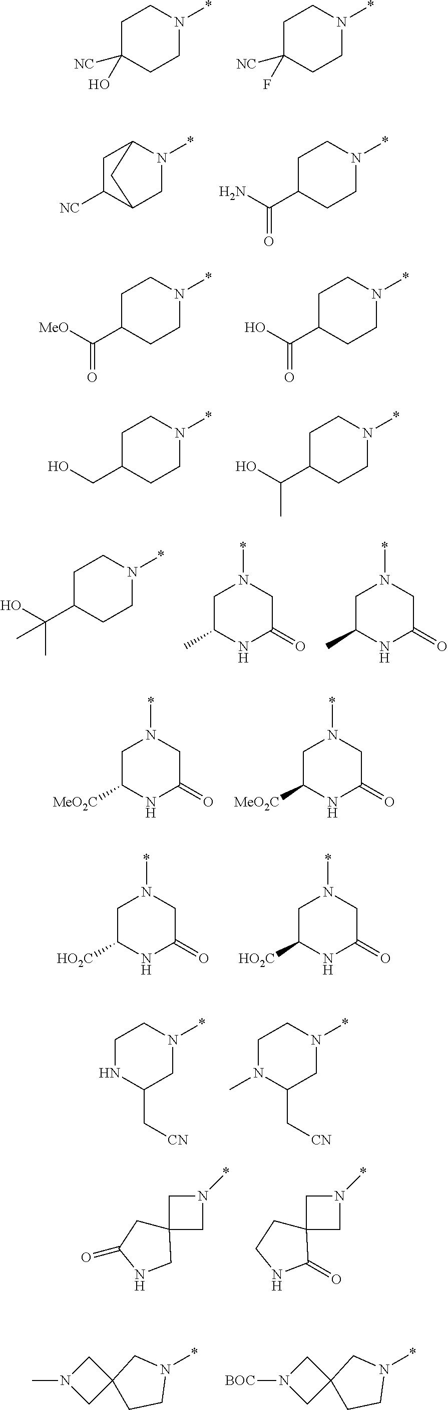

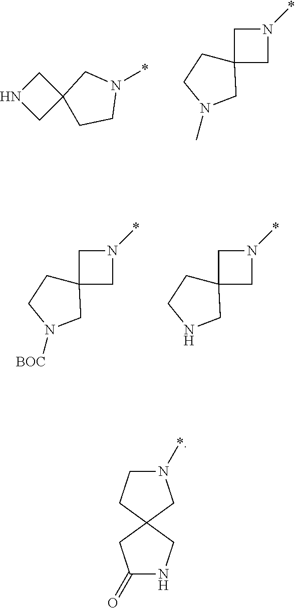

A compound of formula I or la as defined in A, B, C, D, E, F, G, H or I (above), wherein

R¹ is selected from

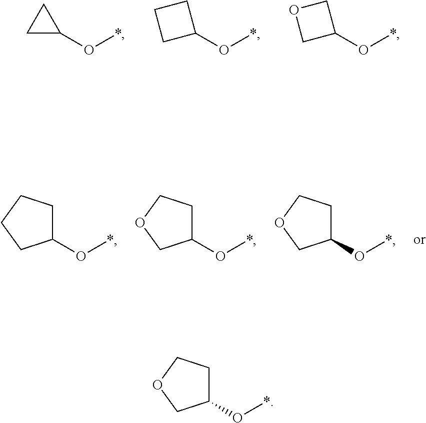

A compound of formula I as defined in A, B, C, D, E, F, G, H or I (above), wherein

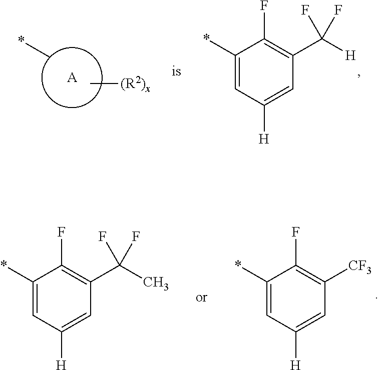

A compound of formula I or Ia as defined in A, B, C, D, E, F, G, H or I (above), wherein

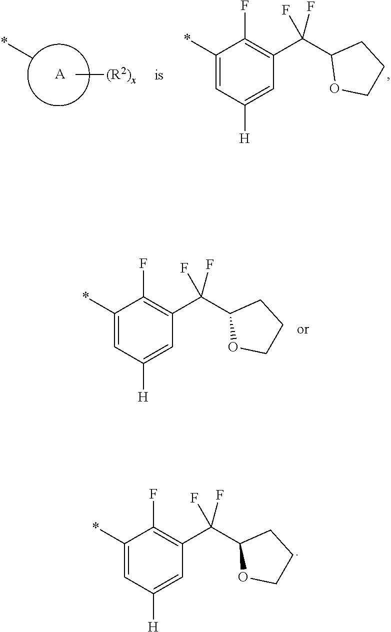

A compound of formula I or Ia as defined in A, B, C, D, E, F, G, H or I (above), wherein

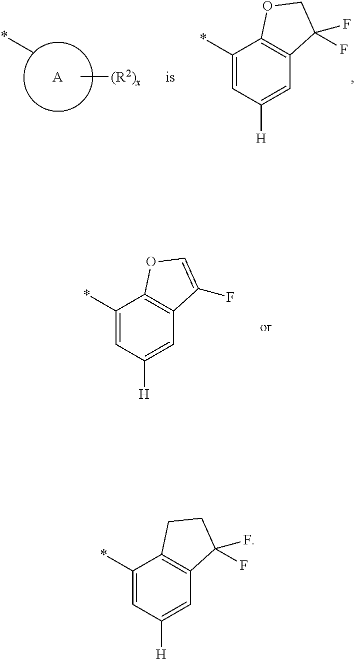

A compound of formula I or Ia as defined in A, B, C, D, E, F, G, H or I (above), wherein

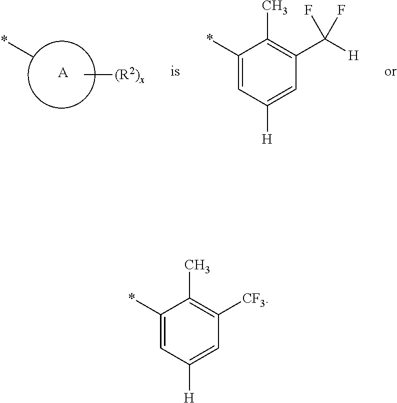

A compound of formula I or Ia as defined in A, B, C, D, E, F, G, H or I (above), wherein

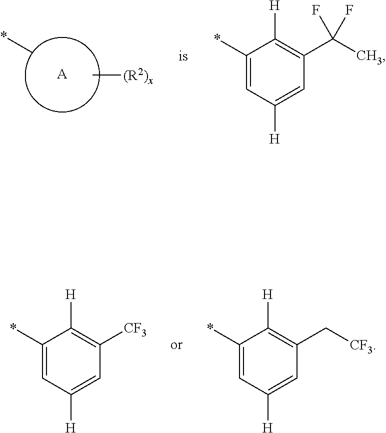

A compound of formula I or Ia as defined in A, B, C, D, E, F, G, H or I (above), wherein

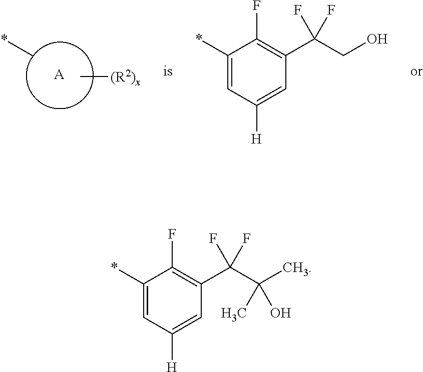

In a particular further embodiment of the first aspect, the present invention covers combinations of two or more of the above mentioned embodiments under the heading “further embodiments of the first aspect of the present invention”.

Further embodiments of this invention can be presented by the following alternative claim set possibility:

1. A compound of general formula (1)

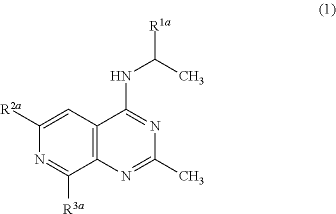

wherein
R^{1a} is selected from the group consisting of
   5-6 membered heteroaryl, 9-10 membered bicyclic heteroaryl or phenyl, all optionally one or more times substituted by
      —H, —OH, —CN, —NO₂, —NH₂, halogen, —COOH, —COO—CH₃, —SF₅, (1E)-2-ethoxyethenyl, [(tert-butoxy)carbonyl]amino, 1H-pyrazol-1-yl, 2-(methylamino)ethoxy, oxolan-3-yloxy, (1-methylpyrrolidin-3-yl)oxy, C₁₋₆-alkyl optionally substituted one or more times with —F and/or —OH and/or —O—C₁₋₆-alkyl or —S—C₁₋₆-alkyl, both optionally substituted one or more times with —F;
R^{2a} is selected from the group consisting of —F, —Cl, —OCH₃, —COOCH₃, —S(═O)₂—CH₃, —O—CH₂—CH₂R⁹, —C(═O)-NHR^{3a}, 2,5-dihydrofuran-3-yl, 4,5-dihydrofuran-2-yl, oxolan-3-yl, oxetan-3-yloxy, cyclopentylamino, 5,6-dihydro-2H-pyran-3-yl, oxan-3-yl, 3,6-dihydro-2H-pyran-4-yl, 1-methyl-1H-pyrazol-4-yl, oxan-4-yl, [(oxetan-2-yl)methyl]amino, —N(R^{3a})—CH(R^{3a})—CH₂—R¹², 1-methylpiperidin-4-yl, 1-methyl-1,2,3,6-tetrahydropyridin-4-yl, 1-oxa-6-azaspiro[3.3]heptan-6-yl, [(oxolan-2-yl)methyl]amino, 2-oxa-6-azaspiro[3.3]heptan-6-yl, (1-methylpyrrolidin-3-yl)oxy, (5-oxopyrrolidin-3-yl)amino, 3-(difluoromethyl)azetidin-1-yl, 2-oxa-6-azaspiro[3.4]octan-6-yl, 3-oxo-1,4-diazepan-1-yl, —R²²—COOC(CH₃)₃, 4-cyano-4-methylpiperidin-1-yl, 2-oxa-6-azaspiro[3.5]nonan-6-yl, 2-oxa-7-azaspiro[3.5]nonan-7-yl, 5-oxo-2,6-diazaspiro[3.4]octan-2-yl, 7-oxo-2,6-diazaspiro[3.4]octan-2-yl, 8-oxo-2,7-diazaspiro[4.4]nonan-2-yl, 4-methyl-2,3-dioxo-1,4-diazepan-1-yl,

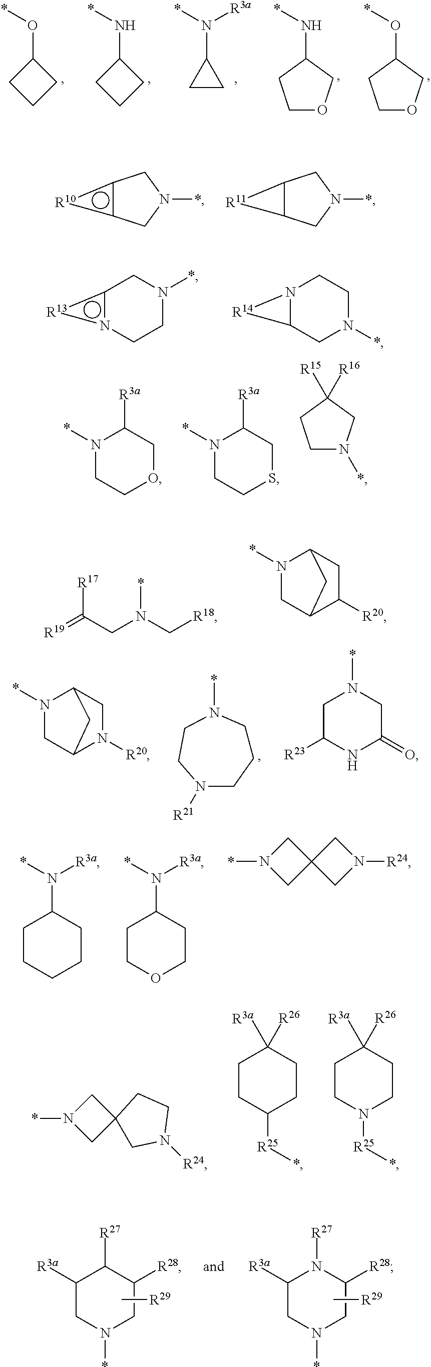

R^{3a} is selected from the group consisting of —H and —CH₃;
R^{4a} is selected from the group consisting of —H and —F;
R^{5a} is selected from the group consisting of —H, —F, —Cl, —Br, —CN, —NO₂, —OH, —CH₂OH, —COO H, —COO—CH₃, —CH₃, —CF₃, —CHF₂, —CF₂—CH₃, —CF₂—CH₂OH, —CF₂—C(CH₃)₂OH, —O—CH₃, —O—CH₂—CHF₂, —O—CF₃, —O—CHF₂, —S—CF₃, —SF_{S}, (1E)-2-ethoxyethenyl, and [(tert-butoxy)carbonyl]amino;
R^{6a} is selected from the group consisting of —H, —F, —Cl, —CH₃, —CHF₂, —O—CH₃, —O—CHF₂, 1H-pyrazol-1-yl, 2-(methylamino)ethoxy, oxolan-3-yloxy, and (1-methylpyrrolidin-3-yl)oxy;
R⁷ is selected from the group consisting of —H, —NH₂, —F, and —Br;
R⁸ is selected from the group consisting of —H, —CH₃, and —F;
R⁹ is selected from the group consisting of —H, —CH₂—CH₃, and —NH—CH₃;
R¹⁰ is selected from the group consisting of

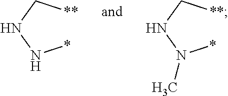

R¹¹ is selected from the group consisting of —CH₂—CH₂—CH₂—, —CH₂—O—CH₂—, —CH₂—CH₂—O—, —N(CH₃)—CH₂—CH₂—, —CH₂—NH—CH₂— and —CH₂—N(R³¹)—CH₂—;
R¹² is selected from the group consisting of —H, —OCH₃, and —N(CH₃)₂;
R¹³ is selected from the group consisting of

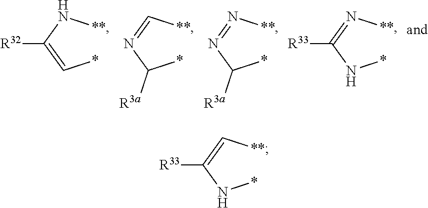

R¹⁴ is selected from the group consisting of —CH₂—C(R^{4a})₂—CH₂—, —CH₂—CH₂—C(═O)— and —CH₂—O—C(═O)—;
R¹⁵ is selected from the group consisting of —H, —OH, —F, —OCH₃, —N(CH₃)₂, —C(═O)—NH₂, —COOH, pyrrolidin-1-yl, —NH—SO₂—R³⁴, —N(R^{3a})—CO—R³⁵, and morpholin-4-yl;
R¹⁶ is selected from the group consisting of —H, —CH₃, —F, and —CH₂—CH₂OH;
R¹⁷ is selected from the group consisting of —H and —N(CH₃)₂;
R¹⁸ is selected from the group consisting of —H and —CH═CH₂;
R¹⁹ is selected from the group consisting of ═CH₂ and ═O;
R²⁰ is selected from the group consisting of —H and —CN;
R²¹ is selected from the group consisting of —H, —CH₃, and —C(═O)—CH₃;
R²² is selected from the group consisting of

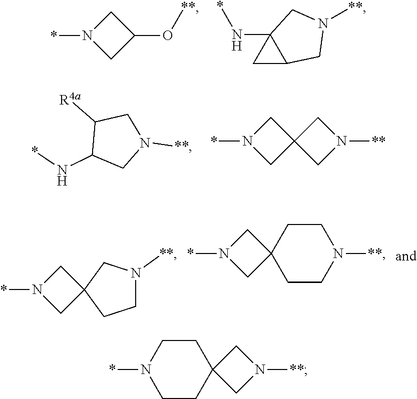

R²³ is selected from the group consisting of —H, —CH₃, and —COOH;
R²⁴ is selected from the group consisting of —CH₃ and —C(═O)—O—C(CH₃)₃;
R²⁵ is selected from the group consisting of —NH— and

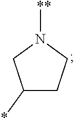

R²⁶ is selected from the group consisting of —H and —OH;
R²⁷ is selected from the group consisting of —H, —CH₃, —CH₂—CH₃, —CN, —CH₂OH, cyclopropyl, —CH₂—CN, —N(CH₃)₂, —C(CH₃)₂OH, —NH—C(═O)—CH₃, —S(═O)₂CH₃, —CH₂—CH₂—OR³⁶, —CH₂—CF₂R^{4a}, —C(═O)—N(R^{3a})₂, oxetane-3-carbonyl, —C(═O)—C(R³⁸)(R³⁹) R^{4a}, and

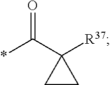

R28 is selected from the group consisting of —H, —CH₃, —OH, —N(CH₃)₂, —S(═O)₂NH₂, and —C(═O)—NHR^{3a};
R²⁹ is selected from the group consisting of —H, —CH₃, and —CH₂OH;
R³¹ is selected from the group consisting of —CH₃ and —C(═O)—CH₃;
R³² is selected from the group consisting of —H and —CF₃;
R³³ is selected from the group consisting of —H, —CN, and —CF₃;
R³⁴ is selected from the group consisting of —CH₃ and cyclopropyl;
R³⁵ is selected from the group consisting of —CH₃, —OCH₃, cyclopropyl, —CH₂—OCH₃, —CHF₂, oxetan-3-yl, and 1-methylazetidin-3-yl;
R³⁶ is selected from the group consisting of —H, —CH₃, and

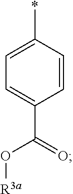

R³⁷ is selected from the group consisting of —H, —F, and —CN;
R³⁸ is selected from the group consisting of —H, —CH₃, —CH₂—NH₂, —CH₂—NH—CH₃, and —CH₂—CH₂—CH₂—CH₂—CH₂—CH₂—CH₂—C(═O)—OR^{3a}; and
R³⁹ is selected from the group consisting of —H, —NH₂, —F, —NH—CH₃, —OCH₃, and —N(CH₃)₂.

or a stereoisomer, a tautomer, an N-oxide, a hydrate, a solvate, or a salt thereof, or a mixture of same.

2. The compound according to claim 1 of general formula (I) in which:
R^{1a} is selected from the group consisting of
   5-chloro-1,3-thiazol-2-yl,
   6-aminopyridin-2-yl,
   5-bromopyridin-3-yl,
   3-(trifluoromethyl)-1,2,4-oxadiazol-5-yl,
   3-fluoro-1-benzofuran-7-yl,

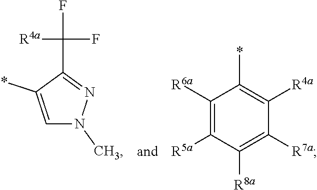

R^{4a} is selected from the group consisting of —H and —F;
R^{5a} is selected from the group consisting of —H, —F, —CI, —Br, —CN, —NO₂, —OH, —CH₂OH, —COOH, —COO—CH₃, —CH₃, —CF₃, —CHF₂, —CF₂—CH₃, —CF₂—CH₂OH, —CF₂—C(CH₃)₂OH, —O—CH₃, —O—CH₂—CHF₂, —O—CF₃, —O—CHF₂, —S—CF₃, —SF₅, (1E)-2-ethoxyethenyl, and [(tert-butoxy)carbonyl]amino;
R^{6a} is selected from the group consisting of —H, —F, —Cl, —CH₃, —CHF₂, —O—CH₃, —O—CHF₂, 1H-pyrazol-1-yl, 2-(methylamino)ethoxy, oxolan-3-yloxy, and (1-methylpyrrolidin-3-yl)oxy;
R⁷ is selected from the group consisting of —H, —NH₂, —F, and —Br; and
R⁸ is selected from the group consisting of —H, —CH₃, and —F;

or a stereoisomer, a tautomer, an N-oxide, a hydrate, a solvate, or a salt thereof, or a mixture of same.

3. The compound according to claim 2 of general formula (I) in which:
R^{1a} is

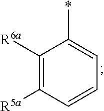

R^{5a} is selected from the group consisting of —CF₃, —CHF₂, —CF₂—CH₃, —CF₂—CH₂OH, and —CF₂—C(CH₃)₂OH; and
R^{6a} is selected from the group consisting of —H, —F, and —CH₃;

or a stereoisomer, a tautomer, an N-oxide, a hydrate, a solvate, or a salt thereof, or a mixture of same.

4. The compound according to claim 1 of general formula (1a)

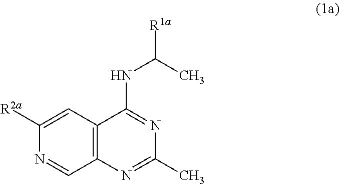

or a stereoisomer, a tautomer, an N-oxide, a hydrate, a solvate, or a salt thereof, or a mixture of same.

5. The compound according to claim 1 of general formula (1b)

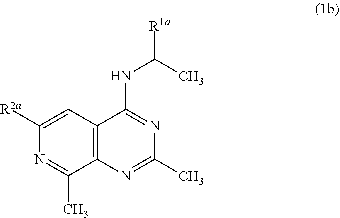

or a stereoisomer, a tautomer, an N-oxide, a hydrate, a solvate, or a salt thereof, or a mixture of same.

6. The compound according to claim 4 of general formula (1a) in which:
R^{1a} is

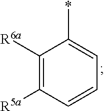

R^{2a} is selected from the group consisting of —F, —Cl, —OCH₃, —COOCH₃, —S(═O)₂—CH₃, —O—CH₂—CH₂R⁹, —C(═O)—NHR^{3a}, 2,5-dihydrofuran-3-yl, 4,5-dihydrofuran-2-yl, oxolan-3-yl, oxetan-3-yloxy, cyclopentylamino, 5,6-dihydro-2H-pyran-3-yl, oxan-3-yl, 3,6-dihydro-2H-pyran-4-yl, 1-methyl-1H-pyrazol-4-yl, oxan-4-yl, [(oxetan-2-yl)methyl]amino, —N(R^{3a})—CH(R^{3a})—CH₂—R¹², 1-methylpiperidin-4-yl, 1-methyl-1,2,3,6-tetrahydropyridin-4-yl, 1-oxa-6-azaspiro[3.3]heptan-6-yl, [(oxolan-2-yl)methyl]amino, 2-oxa-6-azaspiro[3.3]heptan-6-yl, (1-methylpyrrolidin-3-yl)oxy, (5-oxopyrrolidin-3-yl)amino, 3-(difluoromethyl)azetidin-1-yl, 2-oxa-6-azaspiro[3.4]octan-6-yl, 3-oxo-1,4-diazepan-1-yl, —R²²—COOC(CH₃)₃, 4-cyano-4-methylpiperidin-1-yl, 2-oxa-6-azaspiro[3.5]nonan-6-yl, 2-oxa-7-azaspiro[3.5]nonan-7-yl, 5-oxo-2,6-diazaspiro[3.4]octan-2-yl, 7-oxo-2,6-diazaspiro[3.4]octan-2-yl, 8-oxo-2,7-diazaspiro[4.4]nonan-2-yl, 4-methyl-2,3-dioxo-1,4-diazepan-1-yl,

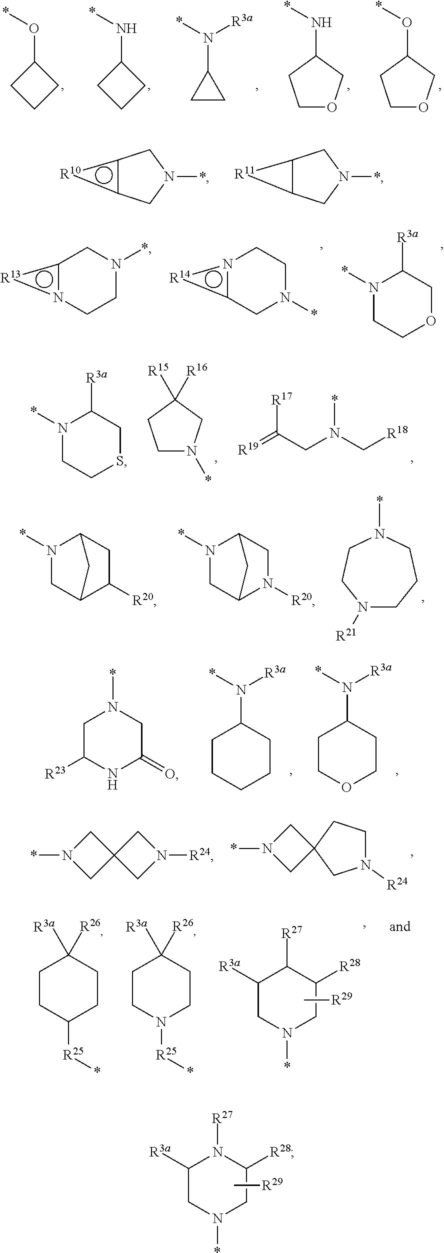

R^{3a} is selected from the group consisting of —H and —CH₃;
R^{4a} is selected from the group consisting of —H and —F;
R^{5a} is selected from the group consisting of —CF₃, —CHF₂, —CF₂—CH₃, —CF₂—CH₂OH, and —CF₂—C(CH₃)₂OH; and
R^{6a} is selected from the group consisting of —H, —F, and —CH₃;
R⁹ is selected from the group consisting of —H, —CH₂—CH₃, and —NH—CH₃;
R¹⁰ is selected from the group consisting of

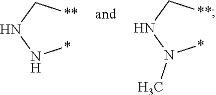

R¹¹ is selected from the group consisting of —CH₂—CH₂—CH₂—, —CH₂—O—CH₂—, —CH₂—CH₂—O—, —N(CH₃)—CH₂—CH₂—, —CH₂—NH—CH₂— and —CH₂—N(R³¹)—CH₂—;
R¹² is selected from the group consisting of —H, —OCH₃, and —N(CH₃)₂;
R¹³ is selected from the group consisting of

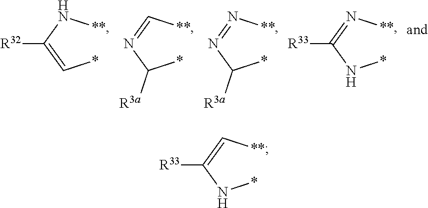

R¹⁴ is selected from the group consisting of —CH₂—C(R^{4a})₂—CH₂—, —CH₂—CH₂—C(═O)— and —CH₂—O—C(═O)—;
R¹⁵ is selected from the group consisting of —H, —OH, —F, —OCH₃, —N(CH₃)₂, —C(═O)—NH₂, —COOH, pyrrolidin-1-yl, —NH—SO₂—R³⁴, —N(R^{3a})—CO—R³⁵, and morpholin-4-yl;
R¹⁶ is selected from the group consisting of —H, —CH₃, —F, and —CH₂—CH₂OH;
R¹⁷ is selected from the group consisting of —H and —N(CH₃)₂;
R¹⁸ is selected from the group consisting of —H and —CH═CH₂;
R¹⁹ is selected from the group consisting of ═CH₂ and ═O;
R²⁰ is selected from the group consisting of —H and —CN;
R²¹ is selected from the group consisting of —H, —CH₃, and —C(═O)—CH₃;
R²² is selected from the group consisting of

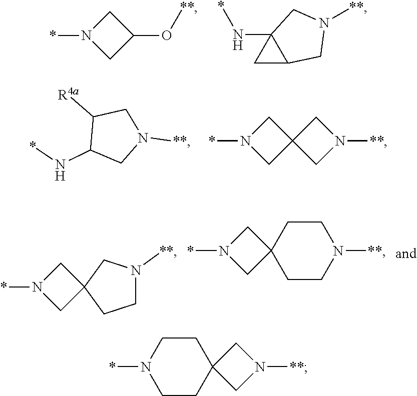

R²³ is selected from the group consisting of —H, —CH₃, and —COOH;
R²⁴ is selected from the group consisting of —CH₃ and —C(═O)—O—C(CH₃)₃;
R²⁵ is selected from the group consisting of —NH— and

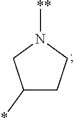

R²⁶ is selected from the group consisting of —H and —OH;
R²⁷ is selected from the group consisting of —H, —CH₃, —CH₂—CH₃, —CN, —CH₂OH, cyclopropyl, —CH₂—CN, —N(CH₃)₂, —C(CH₃)₂OH, —NH—C(═O)—CH₃, —S(═O)₂CH₃, —CH₂—CH₂—OR³⁶, —CH₂—CF₂R^{4a}, —C(═O)—N(R^{3a})₂, oxetane-3-carbonyl, —C(═O)—C(R³⁸)(R³⁹)R^{4a}, and

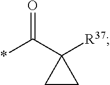

R²⁸ is selected from the group consisting of —H, —CH₃, —OH, —N(CH₃)₂, —S(═O)₂NH₂, and —C(═O)—NHR^{3a};
R²⁹ is selected from the group consisting of —H, —CH₃, and —CH₂OH;
R³¹ is selected from the group consisting of —CH₃ and —C(═O)—CH₃;
R³² is selected from the group consisting of —H and —CF₃;
R³³ is selected from the group consisting of —H, —CN, and —CF₃;
R³⁴ is selected from the group consisting of —CH₃ and cyclopropyl;
R³⁵ is selected from the group consisting of —CH₃, —OCH₃, cyclopropyl, —CH₂—OCH₃, —CHF₂, oxetan-3-yl, and 1-methylazetidin-3-yl;
R³⁶ is selected from the group consisting of —H, —CH₃, and

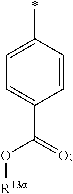

R³⁷ is selected from the group consisting of —H, —F, and —CN;
R³⁸ is selected from the group consisting of —H, —CH₃, —CH₂—NH₂, —CH₂—NH—CH₃, and —CH₂—CH₂—CH₂—CH₂—CH₂—CH₂—CH₂—C(═O)—OR^{3a}; and
R³⁹ is selected from the group consisting of —H, —NH₂, —F, —NH—CH₃, —OCH₃, and —N(CH₃)₂.

or a stereoisomer, a tautomer, an N-oxide, a hydrate, a solvate, or a salt thereof, or a mixture of same.

7. The compound according to claim 5 of general formula (1b) in which:
R^{1a} is

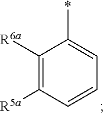

R^{2a} is selected from the group consisting of —F, —Cl, —OCH₃, —COOCH₃, —S(═O)₂—CH₃, —O—CH₂—CH₂R⁹, —C(═O)—NHR^{3a}, 2,5-dihydrofuran-3-yl, 4,5-dihydrofuran-2-yl, oxolan-3-yl, oxetan-3-yloxy, cyclopentylamino, 5,6-dihydro-2H-pyran-3-yl, oxan-3-yl, 3,6-dihydro-2H-pyran-4-yl, 1-methyl-1H-pyrazol-4-yl, oxan-4-yl, [(oxetan-2-yl)methyl]amino, —N(R^{3a})—CH(R^{3a})—CH₂—R¹², 1-methylpiperidin-4-yl, 1-methyl-1,2,3,6-tetrahydropyridin-4-yl, 1-oxa-6-azaspiro[3.3]heptan-6-yl, [(oxolan-2-yl)methyl]amino, 2-oxa-6-azaspiro[3.3]heptan-6-yl, (1-methylpyrrolidin-3-yl)oxy, (5-oxopyrrolidin-3-yl)amino, 3-(difluoromethyl)azetidin-1-yl, 2-oxa-6-azaspiro[3.4]octan-6-yl, 3-oxo-1,4-diazepan-1-yl, —R²²—COOC(CH₃)₃, 4-cyano-4-methylpiperidin-1-yl, 2-oxa-6-azaspiro[3.5]nonan-6-yl, 2-oxa-7-azaspiro[3.5]nonan-7-yl, 5-oxo-2,6-diazaspiro[3.4]octan-2-yl, 7-oxo-2,6-diazaspiro[3.4]octan-2-yl, 8-oxo-2,7-diazaspiro[4.4]nonan-2-yl, 4-methyl-2,3-dioxo-1,4-diazepan-1-yl,

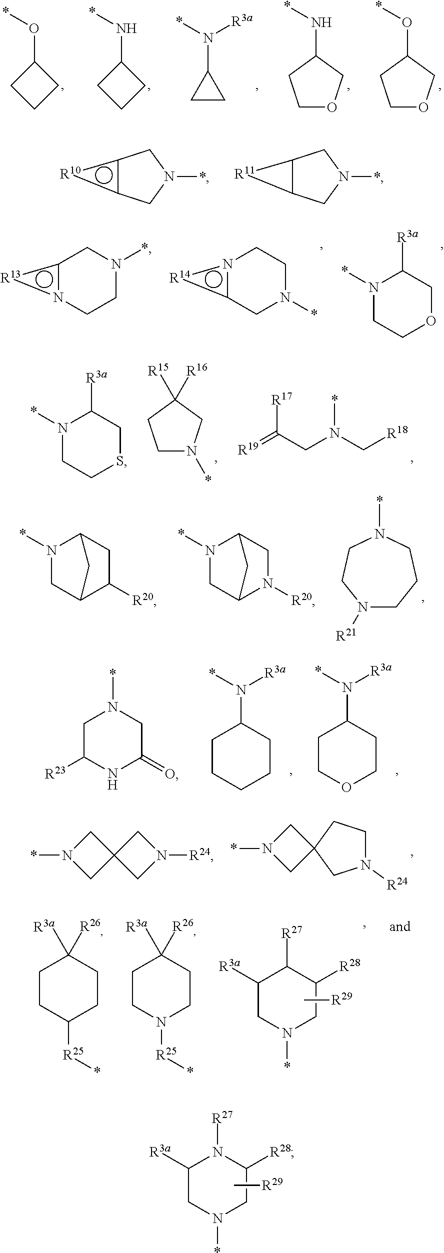

R^{3a} is selected from the group consisting of —H and —CH₃;
R^{4a} is selected from the group consisting of —H and —F;
R^{5a} is selected from the group consisting of —CF₃, —CHF₂, —CF₂—CH₃, —CF₂—CH₂OH, and —CF₂—C(CH₃)₂OH; and
R^{6a} is selected from the group consisting of —H, —F, and —CH₃;
R⁹ is selected from the group consisting of —H, —CH₂—CH₃, and —NH—CH₃;
R¹⁰ is selected from the group consisting of

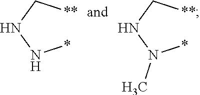

R¹¹ is selected from the group consisting of —CH₂—CH₂—CH₂—, —CH₂—O—CH₂—, —CH₂—CH₂—O—, —N(CH₃)—CH₂—CH₂—, —CH₂—NH—CH₂— and —CH₂—N(R³¹)—CH₂—;
R¹² is selected from the group consisting of —H, —OCH₃, and —N(CH₃)₂;
R¹³ is selected from the group consisting of

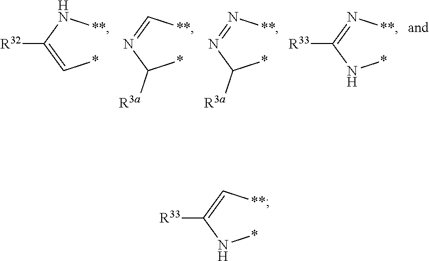

R¹⁴ is selected from the group consisting of —CH₂—C(R^{4a})₂—CH₂—, —CH₂—CH₂—C(═O)— and —CH₂—O—C(═O)—;
R¹⁵ is selected from the group consisting of —H, —OH, —F, —OCH₃, —N(CH₃)₂, —C(═O)—NH₂, —COOH, pyrrolidin-1-yl, —NH—SC₂—R³⁴, —N(R^{3a})—CO—R³⁵, and morpholin-4-yl;
R¹⁶ is selected from the group consisting of —H, —CH₃, —F, and —CH₂—CH₂OH;
R¹⁷ is selected from the group consisting of —H and —N(CH₃)₂;
R¹⁸ is selected from the group consisting of —H and —CH═CH₂;
R¹⁹ is selected from the group consisting of ═CH₂ and ═O;
R²⁰ is selected from the group consisting of —H and —CN;
R²¹ is selected from the group consisting of —H, —CH₃, and —C(═O)—CH₃;
R²² is selected from the group consisting of

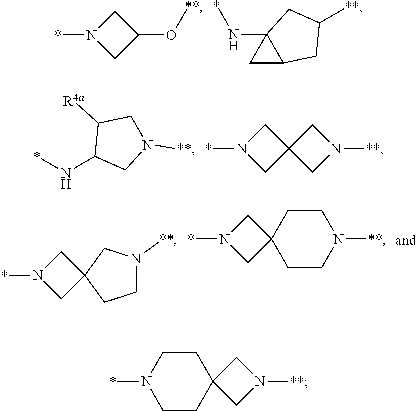

R²³ is selected from the group consisting of —H, —CH₃, and —COOH;
R²⁴ is selected from the group consisting of —CH₃ and —C(═O)—O—C(CH₃)₃;
R²⁵ is selected from the group consisting of —NH— and

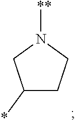

R²⁶ is selected from the group consisting of —H and —OH;
R²⁷ is selected from the group consisting of —H, —CH₃, —CH₂—CH₃, —CN, —CH₂OH, cyclopropyl, —CH₂—CN, —N(CH₃)₂, —C(CH₃)₂OH, —NH—C(═O)—CH₃, —S(═O)₂CH₃, —CH₂—CH₂—OR³⁶, —CH₂—CF₂R^{4a}, —C(═O)—N(R^{3a})₂, oxetane-3-carbonyl, —C(═O)—C(R³⁸)(R³⁹)R^{4a}, and

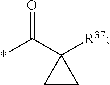

R²⁸ is selected from the group consisting of —H, —CH₃, —OH, —N(CH₃)₂, —S(═O)₂NH₂, and —C(═O)—NHR^{3a};
R²⁹ is selected from the group consisting of —H, —CH₃, and —CH₂OH;
R³¹ is selected from the group consisting of —CH₃ and —C(═O)—CH₃;
R³² is selected from the group consisting of —H and —CF₃;
R³³ is selected from the group consisting of —H, —CN, and —CF₃;
R³⁴ is selected from the group consisting of —CH₃ and cyclopropyl;
R³⁵ is selected from the group consisting of —CH₃, —OCH₃, cyclopropyl, —CH₂—OCH₃, —CHF₂, oxetan-3-yl, and 1-methylazetidin-3-yl;
R³⁶ is selected from the group consisting of —H, —CH₃, and

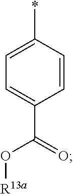

R³⁷ is selected from the group consisting of —H, —F, and —CN;
R³⁸ is selected from the group consisting of —H, —CH₃, —CH₂—NH₂, —CH₂—NH—CH₃, and —CH₂—CH₂—CH₂—CH₂—CH₂—CH₂—CH₂—C(═O)—OR^{3a}; and
R³⁹ is selected from the group consisting of —H, —NH₂, —F, —NH—CH₃, —OCH₃, and —N(CH₃)₂.

or a stereoisomer, a tautomer, an N-oxide, a hydrate, a solvate, or a salt thereof, or a mixture of same.

8. A compound according to claim 1, which is selected from the group consisting of:
N-{(1R)-1-[3-(difluoromethyl)-2-fluorophenyl]ethyl}-6-ethoxy-2-methylpyrido[3,4-d]pyrimidin-4-amine
N-{(1R)-1-[3-(difluoromethyl)-2-fluorophenyl]ethyl}-6-fluoro-2-methylpyrido[3,4-d]pyrimidin-4-amine
N-[(3R)-1-[4-[[(1R)-1-[3-(difluoromethyl)-2-fluoro-phenyl]ethyl]amino]-2-methyl-pyrido[3,4-d]pyrimidin-6-yl]pyrrolidin-3-yl]acetamide
N-[(3S)-1-[4-[[(1R)-1-[3-(difluoromethyl)-2-fluoro-phenyl]ethyl]amino]-2-methyl-pyrido[3,4-d]pyrimidin-6-yl]pyrrolidin-3-yl]acetamide
N-[(1R)-1-[3-(difluoromethyl)-2-fluoro-phenyl]ethyl]-2-methyl-6-pyrrolidin-1-yl-pyrido[3,4-d]pyrimidin-4-amine
N-{(1R)-1-[3-(difluoromethyl)-2-methylphenyl]ethyl}-6-fluoro-2-methylpyrido[3,4-d]pyrimidin-4-amine
N-[(3R)-1-[4-[[(1R)-1-[3-(difluoromethyl)-2-methyl-phenyl]ethyl]amino]-2-methyl-pyrido[3,4-d]pyrimidin-6-yl]pyrrolidin-3-yl]acetamide
N-[(3S)-1-[4-[[(1R)-1-[3-(difluoromethyl)-2-methyl-phenyl]ethyl]amino]-2-methyl-pyrido[3,4-d]pyrimidin-6-yl]pyrrolidin-3-yl]acetamide
N-[(1R)-1-[3-(difluoromethyl)-2-methyl-phenyl]ethyl]-2-methyl-6-pyrrolidin-1-yl-pyrido[3,4-d]pyrimidin-4-amine
6-fluoro-2-methyl-N-[(1R)-1-[3-(trifluoromethyl)phenyl]ethyl]pyrido[3,4-d]pyrimidin-4-amine
N-[(3R)-1-[2-methyl-4-[[(1R)-1-[3-(trifluoromethyl)phenyl]ethyl]amino]pyrido[3,4-d]pyrimidin-6-yl]pyrrolidin-3-yl]acetamide
N-[(3S)-1-[2-methyl-4-[[(1R)-1-[3-(trifluoromethyl)phenyl]ethyl]amino]pyrido[3,4-d]pyrimidin-6-yl]pyrrolidin-3-yl]acetamide
N-[(1R)-1-[3-(1,1-difluoroethyl)phenyl]ethyl]-6-fluoro-2-methyl-pyrido[3,4-d]pyrimidin-4-amine
N-[(3R)-1-[4-[[(1R)-1-[3-(1,1-difluoroethyl)phenyl]ethyl]amino]-2-methyl-pyrido[3,4-d]pyrimidin-6-yl]pyrrolidin-3-yl]acetamide
N-[(3S)-1-[4-[[(1R)-1-[3-(1,1-difluoroethyl)phenyl]ethyl]amino]-2-methyl-pyrido[3,4-d]pyrimidin-6-yl]pyrrolidin-3-yl]acetamide
N-[(1R)-1-[3-(1,1-difluoroethyl)phenyl]ethyl]-6-fluoro-2-methyl-pyrido[3,4-d]pyrimidin-4-amine
N-[(3R)-1-[4-[[(1R)-1-[3-(1,1-difluoroethyl)-2-fluoro-phenyl]ethyl]amino]-2-methyl-pyrido[3,4-d]pyrimidin-6-yl]pyrrolidin-3-yl]acetamide
N-[(3S)-1-[4-[[(1R)-1-[3-(1,1-difluoroethyl)-2-fluoro-phenyl]ethyl]amino]-2-methyl-pyrido[3,4-d]pyrimidin-6-yl]pyrrolidin-3-yl]acetamide
N-{(1R)-1-[3-(difluoromethyl)-2-fluorophenyl]ethyl}-6-fluoro-2,8-dimethylpyrido[3,4-d]pyrimidin-4-amine
N-{(3R)-1-[4-({(1R)-1-[3-(difluoromethyl)-2-fluorophenyl]ethyl}amino)-2,8-dimethylpyrido[3,4-d]pyrimidin-6-yl]pyrrolidin-3-yl}acetamide
N-{(1R)-1-[3-(difluoromethyl)-2-fluorophenyl]ethyl}-6-[(3R)-3-(dimethylamino)pyrrolidin-1-yl]-2,8-dimethylpyrido[3,4-d]pyrimidin-4-amine
1-{4-[4-({(1R)-1-[3-(difluoromethyl)-2-fluorophenyl]ethyl}amino)-2,8-dimethylpyrido[3,4-d]pyrimidin-6-yl]piperazin-1-yl}ethan-1-one
N-{(1R)-1-[3-(difluoromethyl)-2-fluorophenyl]ethyl}-2,8-dimethyl-6-(4-methylpiperazin-1-yl)pyrido[3,4-d]pyrimidin-4-amine
2-[4-({(1R)-1-[3-(difluoromethyl)-2-fluorophenyl]ethyl}amino)-2,8-dimethylpyrido[3,4-d]pyrimidin-6-yl]-2,6-diazaspiro[3.4]octan-7-one
N-{(3S)-1-[4-({(1R)-1-[3-(difluoromethyl)phenyl]ethyl}amino)-2-methylpyrido[3,4-d]pyrimidin-6-yl]pyrrolidin-3-yl}acetamide
N-{(3S)-1-[2-methyl-4-({(1R)-1-[2-methyl-3-(trifluoromethyl)phenyl]ethyl}amino)pyrido[3,4-d]pyrimidin-6-yl]pyrrolidin-3-yl}acetamide
6-ethoxy-2-methyl-N-{(1R)-1-[3-(trifluoromethyl)phenyl]ethyl}pyrido[3,4-d]pyrimidin-4-amine
1-(3-{(1R)-1-[(6-ethoxy-2-methylpyrido[3,4-d]pyrimidin-4-yl)amino]ethyl}-2-fluorophenyl)-1,1-difluoro-2-methylpropan-2-ol
6-ethoxy-N-{(1R)-1-[2-fluoro-3-(trifluoromethyl)phenyl]ethyl}-2-methylpyrido[3,4-d]pyrimidin-4-amine
N-{(1R)-1-[3-(1,1-difluoroethyl)-2-fluorophenyl]ethyl}-6-ethoxy-2-methylpyrido[3,4-d]pyrimidin-4-amine
N-{(1R)-1-[3-(difluoromethyl)-2-methylphenyl]ethyl}-6-ethoxy-2-methylpyrido[3,4-d]pyrimidin-4-amine
6-ethoxy-2-methyl-N-{(1R)-1-[2-methyl-3-(trifluoromethyl)phenyl]ethyl}pyrido[3,4-d]pyrimidin-4-amine
N-{(1R)-1-[3-(difluoromethyl)phenyl]ethyl}-6-ethoxy-2-methylpyrido[3,4-d]pyrimidin-4-amine
N-{(1R)-1-[3-amino-5-(trifluoromethyl)phenyl]ethyl}-6-ethoxy-2-methylpyrido[3,4-d]pyrimidin-4-amine
6-methoxy-2-methyl-N-{(1R)-1-[3-(trifluoromethyl)phenyl]ethyl}pyrido[3,4-d]pyrimidin-4-amine
N-{(1R)-1-[3-(difluoromethyl)-2-fluorophenyl]ethyl}-6-methoxy-2-methylpyrido[3,4-d]pyrimidin-4-amine
N-{(1R)-1-[2-fluoro-3-(trifluoromethyl)phenyl]ethyl}-6-methoxy-2-methylpyrido[3,4-d]pyrimidin-4-amine
N-{(1R)-1-[3-(difluoromethyl)-2-methylphenyl]ethyl}-6-methoxy-2-methylpyrido[3,4-d]pyrimidin-4-amine
6-methoxy-2-methyl-N-{(1R)-1-[2-methyl-3-(trifluoromethyl)phenyl]ethyl}pyrido[3,4-d]pyrimidin-4-amine
N-{(1R)-1-[3-(1,1-difluoroethyl)-2-fluorophenyl]ethyl}-6-methoxy-2-methylpyrido[3,4-d]pyrimidin-4-amine
2,2-difluoro-2-(2-fluoro-3-{(1R)-1-[(6-methoxy-2-methylpyrido[3,4-d]pyrimidin-4-yl)amino]ethyl}phenyl)ethan-1-ol
1,1-difluoro-1-(2-fluoro-3-{(1R)-1-[(6-methoxy-2-methylpyrido[3,4-d]pyrimidin-4-yl)amino]ethyl}phenyl)-2-methylpropan-2-ol
N-{(3R)-1-[4-({(1R)-1-[3-(1,1-difluoro-2-hydroxyethyl)-2-fluorophenyl]ethyl}amino)-2-methylpyrido[3,4-d]pyrimidin-6-yl]pyrrolidin-3-yl}acetamide
N-{(3R)-1-[4-({(1R)-1-[3-(1,1-difluoro-2-hydroxy-2-methylpropyl)-2-fluorophenyl]ethyl}amino)-2-methylpyrido[3,4-d]pyrimidin-6-yl]pyrrolidin-3-yl}acetamide
N-{(3R)-1-[4-({(1R)-1-[2-fluoro-3-(trifluoromethyl)phenyl]ethyl}amino)-2-methylpyrido[3,4-d]pyrimidin-6-yl]pyrrolidin-3-yl}acetamide
N-{(3R)-1-[2-methyl-4-({(1R)-1-[2-methyl-3-(trifluoromethyl)phenyl]ethyl}amino)pyrido[3,4-d]pyrimidin-6-yl]pyrrolidin-3-yl}acetamide
N-{(3R)-1-[4-({(1R)-1-[3-(difluoromethyl)phenyl]ethyl}amino)-2-methylpyrido[3,4-d]pyrimidin-6-yl]pyrrolidin-3-yl}acetamide
N-[(3R)-1-(2-methyl-4-{[(1R)-1-(2-methylphenyl)ethyl]amino}pyrido[3,4-d]pyrimidin-6-yl)pyrrolidin-3-yl]acetamide
N-[(3R)-1-(2-methyl-4-{[(1R)-1-(3-methylphenyl)ethyl]amino}pyrido[3,4-d]pyrimidin-6-yl)pyrrolidin-3-yl]acetamide
N-[(3R)-1-(2-methyl-4-{[(1R)-1-(4-methylphenyl)ethyl]amino}pyrido[3,4-d]pyrimidin-6-yl)pyrrolidin-3-yl]acetamide
N-[(3R)-1-(4-{[(1R)-1-(2-fluorophenyl)ethyl]amino}-2-methylpyrido[3,4-d]pyrimidin-6-yl)pyrrolidin-3-yl]acetamide
N-[(3R)-1-(4-{[(1R)-1-(3-fluorophenyl)ethyl]amino}-2-methylpyrido[3,4-d]pyrimidin-6-yl)pyrrolidin-3-yl]acetamide
N-[(3R)-1-(4-{[(1R)-1-(4-fluorophenyl)ethyl]amino}-2-methylpyrido[3,4-d]pyrimidin-6-yl)pyrrolidin-3-yl]acetamide
N-[(3R)-1-(4-{[(1R)-1-(2-methoxyphenyl)ethyl]amino}-2-methylpyrido[3,4-d]pyrimidin-6-yl)pyrrolidin-3-yl]acetamide
N-[(3R)-1-(4-{[(1R)-1-(3-methoxyphenyl)ethyl]amino}-2-methylpyrido[3,4-d]pyrimidin-6-yl)pyrrolidin-3-yl]acetamide
N-[(3R)-1-(4-{[(1R)-1-(2-chlorophenyl)ethyl]amino}-2-methylpyrido[3,4-d]pyrimidin-6-yl)pyrrolidin-3-yl]acetamide
N-[(3R)-1-(4-{[(1R)-1-(3-chlorophenyl)ethyl]amino}-2-methylpyrido[3,4-d]pyrimidin-6-yl)pyrrolidin-3-yl]acetamide
N-{(3R)-1-[4-({(1RS)-1-[2-(difluoromethyl)phenyl]ethyl}amino)-2-methylpyrido[3,4-d]pyrimidin-6-yl]pyrrolidin-3-yl}acetamide
N-{(3R)-1-[4-({(1RS)-1-[2-(difluoromethoxy)phenyl]ethyl}amino)-2-methylpyrido[3,4-d]pyrimidin-6-yl]pyrrolidin-3-yl}acetamide
N-{(3R)-1-[4-({(1R)-1-[3-(difluoromethoxy)phenyl]ethyl}amino)-2-methylpyrido[3,4-d]pyrimidin-6-yl]pyrrolidin-3-yl}acetamide
N-{(3R)-1-[2-methyl-4-({(1R)-1-[3-(trifluoromethoxy)phenyl]ethyl}amino)pyrido[3,4-d]pyrimidin-6-yl]pyrrolidin-3-yl}acetamide
N-[(3R)-1-(4-{[(1R)-1-(3-bromophenyl)ethyl]amino}-2-methylpyrido[3,4-d]pyrimidin-6-yl)pyrrolidin-3-yl]acetamide
N-[(3R)-1-(2-methyl-4-{[(1R)-1-{3-[(trifluoromethyl)sulfanyl]phenyl}ethyl]amino}pyrido[3,4-d]pyrimidin-6-yl)pyrrolidin-3-yl]acetamide
N-{(3R)-1-[2-methyl-4-({(1R)-1-[3-(pentafluoro-lambda⁶-sulfanyl)phenyl]ethyl}amino)pyrido[3,4-d]pyrimidin-6-yl]pyrrolidin-3-yl}acetamide
methyl 3-[(1R)-1-({6-[(3R)-3-acetamidopyrrolidin-1-yl]-2-methylpyrido[3,4-d]pyrimidin-4-yl}amino)ethyl]benzoate
N-[(3R)-1-(4-{[(1R)-1-(3-cyanophenyl)ethyl]amino}-2-methylpyrido[3,4-d]pyrimidin-6-yl)pyrrolidin-3-yl]acetamide
N-[(3R)-1-(2-methyl-4-{[(1R)-1-(3-nitrophenyl)ethyl]amino}pyrido[3,4-d]pyrimidin-6-yl)pyrrolidin-3-yl]acetamide
tert-butyl {3-[(1RS)-1-({6-[(3R)-3-acetamidopyrrolidin-1-yl]-2-methylpyrido[3,4-d]pyrimidin-4-yl}amino)ethyl]phenyl}carbamate
N-[(3R)-1-(4-{[(1R)-1-(4-fluoro-3-methylphenyl)ethyl]amino}-2-methylpyrido[3,4-d]pyrimidin-6-yl)pyrrolidin-3-yl]acetamide
N-[(3R)-1-(4-{[(1R)-1-(2,3-difluorophenyl)ethyl]amino}-2-methylpyrido[3,4-d]pyrimidin-6-yl)pyrrolidin-3-yl]acetamide
N-[(3R)-1-(4-{[(1R)-1-(3,4-difluorophenyl)ethyl]amino}-2-methylpyrido[3,4-d]pyrimidin-6-yl)pyrrolidin-3-yl]acetamide
N-[(3R)-1-(4-{[(1R)-1-(2,4-difluorophenyl)ethyl]amino}-2-methylpyrido[3,4-d]pyrimidin-6-yl)pyrrolidin-3-yl]acetamide
N-[(3R)-1-(4-{[(1RS)-1-(3,5-difluorophenyl)ethyl]amino}-2-methylpyrido[3,4-d]pyrimidin-6-yl)pyrrolidin-3-yl]acetamide
N-[(3R)-1-(4-{[(1RS)-1-(2,6-difluorophenyl)ethyl]amino}-2-methylpyrido[3,4-d]pyrimidin-6-yl)pyrrolidin-3-yl]acetamide
N-[(3R)-1-(4-{[(1RS)-1-(2,5-difluorophenyl)ethyl]amino}-2-methylpyrido[3,4-d]pyrimidin-6-yl)pyrrolidin-3-yl]acetamide
N-[(3R)-1-(4-{[(1R)-1-(5-bromo-2-methylphenyl)ethyl]amino}-2-methylpyrido[3,4-d]pyrimidin-6-yl)pyrrolidin-3-yl]acetamide
N-[(3R)-1-(4-{[(1R)-1-(3-bromo-5-fluorophenyl)ethyl]amino}-2-methylpyrido[3,4-d]pyrimidin-6-yl)pyrrolidin-3-yl]acetamide
N-[(3R)-1-(4-{[(1R)-1-(3-bromo-4-fluorophenyl)ethyl]amino}-2-methylpyrido[3,4-d]pyrimidin-6-yl)pyrrolidin-3-yl]acetamide
N-[(3R)-1-(4-{[(1R)-1-(3-bromo-2-fluorophenyl)ethyl]amino}-2-methylpyrido[3,4-d]pyrimidin-6-yl)pyrrolidin-3-yl]acetamide
N-[(3R)-1-(4-{[(1R)-1-(5-bromo-2-fluorophenyl)ethyl]amino}-2-methylpyrido[3,4-d]pyrimidin-6-yl)pyrrolidin-3-yl]acetamide
N-[(3R)-1-(4-{[(1R)-1-(5-bromo-2-methoxyphenyl)ethyl]amino}-2-methylpyrido[3,4-d]pyrimidin-6-yl)pyrrolidin-3-yl]acetamide
N-[(3R)-1-(4-{[(1R)-1-(3-fluoro-1-benzofuran-7-yl)ethyl]amino}-2-methylpyrido[3,4-d]pyrimidin-6-yl)pyrrolidin-3-yl]acetamide.
N-[(3R)-1-(4-{[(1S)-1-(3-fluoro-1-benzofuran-7-yl)ethyl]amino}-2-methylpyrido[3,4-d]pyrimidin-6-yl)pyrrolidin-3-yl]acetamide
N-{(3R)-1-[2-methyl-4-({(1RS)-1-[2-(1H-pyrazol-1-yl)phenyl]ethyl}amino)pyrido[3,4-d]pyrimidin-6-yl]pyrrolidin-3-yl}acetamide
N-{(3R)-1-[4-({(1RS)-1-[3-(difluoromethyl)-1-methyl-1H-pyrazol-4-yl]ethyl}amino)-2-methylpyrido[3,4-d]pyrimidin-6-yl]pyrrolidin-3-yl}acetamide
N-{(3R)-1-[2-methyl-4-({(1RS)-1-[1-methyl-3-(trifluoromethyl)-1H-pyrazol-4-yl]ethyl}amino)pyrido[3,4-d]pyrimidin-6-yl]pyrrolidin-3-yl}acetamide
N-[(3R)-1-(4-{[(1RS)-1-(5-chloro-1,3-thiazol-2-yl)ethyl]amino}-2-methylpyrido[3,4-d]pyrimidin-6-yl)pyrrolidin-3-yl]acetamide
N-{(3R)-1-[2-methyl-4-({(1RS)-1-[3-(trifluoromethyl)-1,2,4-oxadiazol-5-yl]ethyl}amino)pyrido[3,4-d]pyrimidin-6-yl]pyrrolidin-3-yl}acetamide
N-[(3R)-1-(4-{[(1R)-1-(5-bromopyridin-3-yl)ethyl]amino}-2-methylpyrido[3,4-d]pyrimidin-6-yl)pyrrolidin-3-yl]acetamide
N-[(3R)-1-(4-{[(1R)-1-(6-aminopyridin-2-yl)ethyl]amino}-2-methylpyrido[3,4-d]pyrimidin-6-yl)pyrrolidin-3-yl]acetamide
N-{(3R)-1-[4-({(1R)-1-[3-amino-5-(trifluoromethyl)phenyl]ethyl}amino)-2-methylpyrido[3,4-d]pyrimidin-6-yl]pyrrolidin-3-yl}acetamide
N-[(3R)-1-(4-{[1-(3-aminophenyl)ethyl]amino}-2-methylpyrido[3,4-d]pyrimidin-6-yl)pyrrolidin-3-yl]acetamide (mixture of stereoisomers)
tert-butyl {3-[(1S)-1-({6-[(3R)-3-acetamidopyrrolidin-1-yl]-2-methylpyrido[3,4-d]pyrimidin-4-yl}amino)ethyl]phenyl}carbamate
tert-butyl {3-[(1R)-1-({6-[(3R)-3-acetamidopyrrolidin-1-yl]-2-methylpyrido[3,4-d]pyrimidin-4-yl}amino)ethyl]phenyl}carbamate
N-[(3R)-1-(4-{[(1S)-1-(3-aminophenyl)ethyl]amino}-2-methylpyrido[3,4-d]pyrimidin-6-yl)pyrrolidin-3-yl]acetamide
N-[(3R)-1-(4-{[(1R)-1-(3-aminophenyl)ethyl]amino}-2-methylpyrido[3,4-d]pyrimidin-6-yl)pyrrolidin-3-yl]acetamide
N-[(3R)-1-(4-{[(1R)-1-(3,5-difluorophenyl)ethyl]amino}-2-methylpyrido[3,4-d]pyrimidin-6-yl)pyrrolidin-3-yl]acetamide
N-[(3R)-1-(4-{[(1S)-1-(3,5-difluorophenyl)ethyl]amino}-2-methylpyrido[3,4-d]pyrimidin-6-yl)pyrrolidin-3-yl]acetamide
N-[(3R)-1-(4-{[(1S)-1-(2,6-difluorophenyl)ethyl]amino}-2-methylpyrido[3,4-d]pyrimidin-6-yl)pyrrolidin-3-yl]acetamide
N-[(3R)-1-(4-{[(1R)-1-(2,6-difluorophenyl)ethyl]amino}-2-methylpyrido[3,4-d]pyrimidin-6-yl)pyrrolidin-3-yl]acetamide
N-[(3R)-1-(4-{[(1R)-1-(2,5-difluorophenyl)ethyl]amino}-2-methylpyrido[3,4-d]pyrimidin-6-yl)pyrrolidin-3-yl]acetamide
N-[(3R)-1-(4-{[(1S)-1-(2,5-difluorophenyl)ethyl]amino}-2-methylpyrido[3,4-d]pyrimidin-6-yl)pyrrolidin-3-yl]acetamide
3-[(1R)-1-({6-[(3R)-3-acetamidopyrrolidin-1-yl]-2-methylpyrido[3,4-d]pyrimidin-4-yl}amino)ethyl]benzoic acid
N-{(3R)-1-[4-({(1R)-1-[3-(hydroxymethyl)phenyl]ethyl}amino)-2-methylpyrido[3,4-d]pyrimidin-6-yl]pyrrolidin-3-yl}acetamide
N-[(3R)-1-(4-{[(1R)-1-(3-hydroxyphenyl)ethyl]amino}-2-methylpyrido[3,4-d]pyrimidin-6-yl)pyrrolidin-3-yl]acetamide
N-{(3R)-1-[4-({(1R)-1-[3-(2,2-difluoroethoxy)phenyl]ethyl}amino)-2-methylpyrido[3,4-d]pyrimidin-6-yl]pyrrolidin-3-yl}acetamide
N-[(3R)-1-(4-{[(1R)-1-{3-[(E)-2-ethoxyethenyl]phenyl}ethyl]amino}-2-methylpyrido[3,4-d]pyrimidin-6-yl)pyrrolidin-3-yl]acetamide
N-{(1R)-1-[3-(difluoromethyl)phenyl]ethyl}-2-methyl-6-(4-methylpiperazin-1-yl)pyrido[3,4-d]pyrimidin-4-amine
N-{(1R)-1-[3-(difluoromethyl)-2-methylphenyl]ethyl}-2-methyl-6-(4-methylpiperazin-1-yl)pyrido[3,4-d]pyrimidin-4-amine
N-{(1R)-1-[3-(1,1-difluoroethyl)phenyl]ethyl}-2-methyl-6-(4-methylpiperazin-1-yl)pyrido[3,4-d]pyrimidin-4-amine
N-{(1R)-1-[3-(1,1-difluoroethyl)-2-fluorophenyl]ethyl}-2-methyl-6-(4-methylpiperazin-1-yl)pyrido[3,4-d]pyrimidin-4-amine
N-{(1R)-1-[2-fluoro-3-(trifluoromethyl)phenyl]ethyl}-2-methyl-6-(4-methylpiperazin-1-yl)pyrido[3,4-d]pyrimidin-4-amine
2,2-difluoro-2-{2-fluoro-3-[(1R)-1-{[2-methyl-6-(4-methylpiperazin-1-yl)pyrido[3,4-d]pyrimidin-4-yl]amino}ethyl]phenyl}ethan-1-ol
1,1-difluoro-1-{2-fluoro-3-[(1R)-1-{[2-methyl-6-(4-methylpiperazin-1-yl)pyrido[3,4-d]pyrimidin-4-yl]amino}ethyl]phenyl}-2-methylpropan-2-ol
N-{(1R)-1-[3-amino-5-(trifluoromethyl)phenyl]ethyl}-2-methyl-6-(4-methylpiperazin-1-yl)pyrido[3,4-d]pyrimidin-4-amine
N-{(1R)-1-[3-(difluoromethyl)phenyl]ethyl}-6-[(3R)-3-(dimethylamino)pyrrolidin-1-yl]-2-methylpyrido[3,4-d]pyrimidin-4-amine
N-{(1R)-1-[3-(1,1-difluoroethyl)phenyl]ethyl}-6-[(3R)-3-(dimethylamino)pyrrolidin-1-yl]-2-methylpyrido[3,4-d]pyrimidin-4-amine
N-{(1R)-1-[3-(1,1-difluoroethyl)-2-fluorophenyl]ethyl}-6-[(3R)-3-(dimethylamino)pyrrolidin-1-yl]-2-methylpyrido[3,4-d]pyrimidin-4-amine
6-[(3R)-3-(dimethylamino)pyrrolidin-1-yl]-N-{(1R)-1-[2-fluoro-3-(trifluoromethyl)phenyl]ethyl}-2-methylpyrido[3,4-d]pyrimidin-4-amine
N-{(1R)-1-[3-(difluoromethyl)-2-methylphenyl]ethyl}-6-[(3R)-3-(dimethylamino)pyrrolidin-1-yl]-2-methylpyrido[3,4-d]pyrimidin-4-amine
2-{3-[(1R)-1-({6-[(3R)-3-(dimethylamino)pyrrolidin-1-yl]-2-methylpyrido[3,4-d]pyrimidin-4-yl}amino)ethyl]-2-fluorophenyl}-2,2-difluoroethan-1-ol
1-{3-[(1R)-1-({6-[(3R)-3-(dimethylamino)pyrrolidin-1-yl]-2-methylpyrido[3,4-d]pyrimidin-4-yl}amino)ethyl]-2-fluorophenyl}-1,1-difluoro-2-methylpropan-2-ol
2-[4-({(1R)-1-[3-(difluoromethyl)phenyl]ethyl}amino)-2-methylpyrido[3,4-d]pyrimidin-6-yl]-2,6-diazaspiro[3.4]octan-7-one
2-[4-({(1R)-1-[3-(difluoromethyl)-2-methylphenyl]ethyl}amino)-2-methylpyrido[3,4-d]pyrimidin-6-yl]-2,6-diazaspiro[3.4]octan-7-one
2-[4-({(1R)-1-[3-(1,1-difluoroethyl)phenyl]ethyl}amino)-2-methylpyrido[3,4-d]pyrimidin-6-yl]-2,6-diazaspiro[3.4]octan-7-one
2-[4-({(1R)-1-[3-(1,1-difluoroethyl)-2-fluorophenyl]ethyl}amino)-2-methylpyrido[3,4-d]pyrimidin-6-yl]-2,6-diazaspiro[3.4]octan-7-one
2-[4-({(1R)-1-[2-fluoro-3-(trifluoromethyl)phenyl]ethyl}amino)-2-methylpyrido[3,4-d]pyrimidin-6-yl]-2,6-diazaspiro[3.4]octan-7-one
2-[4-({(1R)-1-[3-(1,1-difluoro-2-hydroxyethyl)-2-fluorophenyl]ethyl}amino)-2-methylpyrido[3,4-d]pyrimidin-6-yl]-2,6-diazaspiro[3.4]octan-7-one
2-[4-({(1R)-1-[3-amino-5-(trifluoromethyl)phenyl]ethyl}amino)-2-methylpyrido[3,4-d]pyrimidin-6-yl]-2,6-diazaspiro[3.4]octan-7-one
1-{4-[4-({(1R)-1-[3-(difluoromethyl)phenyl]ethyl}amino)-2-methylpyrido[3,4-d]pyrimidin-6-yl]piperazin-1-yl}ethan-1-one
1-{4-[4-({(1R)-1-[3-(1,1-difluoroethyl)phenyl]ethyl}amino)-2-methylpyrido[3,4-d]pyrimidin-6-yl]piperazin-1-yl}ethan-1-one
1-{4-[4-({(1R)-1-[3-(1,1-difluoroethyl)-2-fluorophenyl]ethyl}amino)-2-methylpyrido[3,4-d]pyrimidin-6-yl]piperazin-1-yl}ethan-1-one
1-{4-[4-({(1R)-1-[3-(difluoromethyl)-2-methylphenyl]ethyl}amino)-2-methylpyrido[3,4-d]pyrimidin-6-yl]piperazin-1-yl}ethan-1-one
1-{4-[4-({(1R)-1-[2-fluoro-3-(trifluoromethyl)phenyl]ethyl}amino)-2-methylpyrido[3,4-d]pyrimidin-6-yl]piperazin-1-yl}ethan-1-one
1-{4-[4-({(1R)-1-[3-(1,1-difluoro-2-hydroxy-2-methylpropyl)-2-fluorophenyl]ethyl}amino)-2-methylpyrido[3,4-d]pyrimidin-6-yl]piperazin-1-yl}ethan-1-one
1-{4-[4-({(1R)-1-[3-amino-5-(trifluoromethyl)phenyl]ethyl}amino)-2-methylpyrido[3,4-d]pyrimidin-6-yl]piperazin-1-yl}ethan-1-one
N⁴-{(1R)-1-[3-(difluoromethyl)-2-fluorophenyl]ethyl}-N⁶-ethyl-2-methylpyrido[3,4-d]pyrimidine-4,6-diamine
N⁶-cyclopropyl-N⁴-{(1R)-1-[3-(difluoromethyl)-2-fluorophenyl]ethyl}-2-methylpyrido[3,4-d]pyrimidine-4,6-diamine
N⁴-{(1R)-1-[3-(difluoromethyl)-2-fluorophenyl]ethyl}-2-methyl-N⁶-(propan-2-yl)pyrido[3,4-d]pyrimidine-4,6-diamine
N⁴-{(1R)-1-[3-(difluoromethyl)-2-fluorophenyl]ethyl}-N⁶-ethyl-N⁶,2-dimethylpyrido[3,4-d]pyrimidine-4,6-diamine
N⁴-{(1R)-1-[3-(difluoromethyl)-2-fluorophenyl]ethyl}-N⁶,2-dimethyl-N⁶-(prop-2-en-1-yl)pyrido[3,4-d]pyrimidine-4,6-diamine
N⁶-cyclopropyl-N⁴-{(1R)-1-[3-(difluoromethyl)-2-fluorophenyl]ethyl}-N⁶,2-dimethylpyrido[3,4-d]pyrimidine-4,6-diamine
N⁶-cyclobutyl-N⁴-{(1R)-1-[3-(difluoromethyl)-2-fluorophenyl]ethyl}-2-methylpyrido[3,4-d]pyrimidine-4,6-diamine
N⁴-{(1R)-1-[3-(difluoromethyl)-2-fluorophenyl]ethyl}-N⁶,2-dimethyl-N⁶-(propan-2-yl)pyrido[3,4-d]pyrimidine-4,6-diamine
N⁴-{(1R)-1-[3-(difluoromethyl)-2-fluorophenyl]ethyl}-N⁶-(2-methoxyethyl)-2-methylpyrido[3,4-d]pyrimidine-4,6-diamine
N-{(1R)-1-[3-(difluoromethyl)-2-fluorophenyl]ethyl}-2-methyl-6-(piperidin-1-yl)pyrido[3,4-d]pyrimidin-4-amine
N⁶-cyclopentyl-N⁴-{(1R)-1-[3-(difluoromethyl)-2-fluorophenyl]ethyl}-2-methylpyrido[3,4-d]pyrimidine-4,6-diamine
N-{(1R)-1-[43-(difluoromethyl)-2-fluorophenyl]ethyl}-2-methyl-6-(piperazin-1-yl)pyrido[3,4-d]pyrimidin-4-amine
(3S)-1-[4-({(1R)-1-[3-(difluoromethyl)-2-fluorophenyl]ethyl}amino)-2-methylpyrido[3,4-d]pyrimidin-6-yl]pyrrolidin-3-ol
(3R)-1-[4-({(1R)-1-[3-(difluoromethyl)-2-fluorophenyl]ethyl}amino)-2-methylpyrido[3,4-d]pyrimidin-6-yl]pyrrolidin-3-ol
N-{(1R)-1-[3-(difluoromethyl)-2-fluorophenyl]ethyl}-2-methyl-6-(morpholin-4-yl)pyrido[3,4-d]pyrimidin-4-amine
N⁴-{(1R)-1-[3-(difluoromethyl)-2-fluorophenyl]ethyl}-2-methyl-N⁶-{[(2RS)-oxetan-2-yl]methyl}pyrido[3,4-d]pyrimidine-4,6-diamine
N⁴-{(1R)-1-[3-(difluoromethyl)-2-fluorophenyl]ethyl}-2-methyl-N⁶-[(3R)-oxolan-3-yl]pyrido[3,4-d]pyrimidine-4,6-diamine
N⁴-{(1R)-1-[3-(difluoromethyl)-2-fluorophenyl]ethyl}-N⁶-(2-methoxyethyl)-N⁶,2-dimethylpyrido[3,4-d]pyrimidine-4,6-diamine
N⁴-{(1R)-1-[3-(difluoromethyl)-2-fluorophenyl]ethyl}-2-methyl-N⁶,N⁶-di(prop-2-en-1-yl)pyrido[3,4-d]pyrimidine-4,6-diamine
6-[2-azabicyclo[2.2.1]heptan-2-yl]-N-{(1R)-1-[3-(difluoromethyl)-2-fluorophenyl]ethyl}-2-methylpyrido[3,4-d]pyrimidin-4-amine (mixture of stereoisomers)
N-{(1R)-1-[3-(difluoromethyl)-2-fluorophenyl]ethyl}-2-methyl-6-(1-oxa-6-azaspiro[3.3]heptan-6-yl)pyrido[3,4-d]pyrimidin-4-amine
N-{(1R)-1-[3-(difluoromethyl)-2-fluorophenyl]ethyl}-2-methyl-6-(2-oxa-6-azaspiro[3.3]heptan-6-yl)pyrido[3,4-d]pyrimidin-4-amine
N⁶-cyclohexyl-N⁴-{(1R)-1-[3-(difluoromethyl)-2-fluorophenyl]ethyl}-2-methylpyrido[3,4-d]pyrimidine-4,6-diamine
4-{[4-({(1R)-1-[3-(difluoromethyl)-2-fluorophenyl]ethyl}amino)-2-methylpyrido[3,4-d]pyrimidin-6-yl]amino}pyrrolidin-2-one (mixture of stereoisomers)
4-[4-({(1R)-1-[3-(difluoromethyl)-2-fluorophenyl]ethyl}amino)-2-methylpyrido[3,4-d]pyrimidin-6-yl]piperazin-2-one
6-(1,4-diazepan-1-yl)-N-{(1R)-1-[3-(difluoromethyl)-2-fluorophenyl]ethyl}-2-methylpyrido[3,4-d]pyrimidin-4-amine
N-{(1R)-1-[3-(difluoromethyl)-2-fluorophenyl]ethyl}-2-methyl-6-(4-methylpiperazin-1-yl)pyrido[3,4-d]pyrimidin-4-amine
N-{(1R)-1-[3-(difluoromethyl)-2-fluorophenyl]ethyl}-2-methyl-6-[(3R)-3-methylmorpholin-4-yl]pyrido[3,4-d]pyrimidin-4-amine
N-{(1R)-1-[3-(difluoromethyl)-2-fluorophenyl]ethyl}-2-methyl-6-[(3S)-3-methylmorpholin-4-yl]pyrido[3,4-d]pyrimidin-4-amine
(3R)-1-[4-({(1R)-1-[3-(difluoromethyl)-2-fluorophenyl]ethyl}amino)-2-methylpyrido[3,4-d]pyrimidin-6-yl]piperidin-3-ol
(3S)-1-[4-({(1R)-1-[3-(difluoromethyl)-2-fluorophenyl]ethyl}amino)-2-methylpyrido[3,4-d]pyrimidin-6-yl]piperidin-3-ol
N⁴-{(1R)-1-[3-(difluoromethyl)-2-fluorophenyl]ethyl}-2-methyl-N⁶-(oxan-4-yl)pyrido[3,4-d]pyrimidine-4,6-diamine
N⁴-{(1R)-1-[3-(difluoromethyl)-2-fluorophenyl]ethyl}-2-methyl-N⁶-{[(2R)-oxolan-2-yl]methyl}pyrido[3,4-d]pyrimidine-4,6-diamine
N-{(1R)-1-[3-(difluoromethyl)-2-fluorophenyl]ethyl}-6-[(3S)-3-methoxypyrrolidin-1-yl]-2-methylpyrido[3,4-d]pyrimidin-4-amine
N⁴-{(1R)-1-[3-(difluoromethyl)-2-fluorophenyl]ethyl}-N⁶-[2-(dimethylamino)ethyl]-N⁶,2-dimethylpyrido[3,4-d]pyrimidine-4,6-diamine
N-{(1R)-1-[3-(difluoromethyl)-2-fluorophenyl]ethyl}-2-methyl-6-(thiomorpholin-4-yl)pyrido[3,4-d]pyrimidin-4-amine
6-[3-(difluoromethyl)azetidin-1-yl]-N-{(1R)-1-[3-(difluoromethyl)-2-fluorophenyl]ethyl}-2-methylpyrido[3,4-d]pyrimidin-4-amine
N-{(1R)-1-[3-(difluoromethyl)-2-fluorophenyl]ethyl}-6-(3,3-difluoropyrrolidin-1-yl)-2-methylpyrido[3,4-d]pyrimidin-4-amine
N-{(1R)-1-[3-(difluoromethyl)-2-fluorophenyl]ethyl}-6-(2,6-dihydropyrrolo[3,4-c]pyrazol-5(4H)-yl)-2-methylpyrido[3,4-d]pyrimidin-4-amine
1-[4-({(1R)-1-[3-(difluoromethyl)-2-fluorophenyl]ethyl}amino)-2-methylpyrido[3,4-d]pyrimidin-6-yl]piperidine-4-carbonitrile
N-{(1R)-1-[3-(difluoromethyl)-2-fluorophenyl]ethyl}-6-[hexahydrocyclopenta[c]pyrrol-2(1H)-yl]-2-methylpyrido[3,4-d]pyrimidin-4-amine (mixture of stereoisomers)
N-{(1R)-1-[3-(difluoromethyl)-2-fluorophenyl]ethyl}-6-[hexahydropyrrolo[3,4-c]pyrrol-2(1H)-yl]-2-methylpyrido[3,4-d]pyrimidin-4-amine (mixture of stereoisomers)
N-{(1R)-1-[3-(difluoromethyl)-2-fluorophenyl]ethyl}-2-methyl-6-[(3aR,6aS)-tetrahydro-1H-furo[3,4-c]pyrrol-5(3H)-yl]pyrido[3,4-d]pyrimidin-4-amine
N-{(1R)-1-[3-(difluoromethyl)-2-fluorophenyl]ethyl}-6-[(3a RS,6a RS)-hexahydro-5H-furo[2,3-c]pyrrol-5-yl]-2-methylpyrido[3,4-d]pyrimidin-4-amine (mixture of stereisomers)
N-{(1R)-1-[3-(difluoromethyl)-2-fluorophenyl]ethyl}-2-methyl-6-(2-oxa-6-azaspiro[3.4]octan-6-yl)pyrido[3,4-d]pyrimidin-4-amine
N⁶-cyclohexyl-N⁴-{(1R)-1-[3-(difluoromethyl)-2-fluorophenyl]ethyl}-N⁶,2-dimethylpyrido[3,4-d]pyrimidine-4,6-diamine
4-[4-({(1R)-1-[3-(difluoromethyl)-2-fluorophenyl]ethyl}amino)-2-methylpyrido[3,4-d]pyrimidin-6-yl]-1,4-diazepan-2-one
(3S)-1-[4-({(1R)-1-[3-(difluoromethyl)-2-fluorophenyl]ethyl}amino)-2-methylpyrido[3,4-d]pyrimidin-6-yl]pyrrolidine-3-carboxamide
(6R)-4-[4-({(1R)-1-[3-(difluoromethyl)-2-fluorophenyl]ethyl}amino)-2-methylpyrido[3,4-d]pyrimidin-6-yl]-6-methylpiperazin-2-one
(6S)-4-[4-({(1R)-1-[3-(difluoromethyl)-2-fluorophenyl]ethyl}amino)-2-methylpyrido[3,4-d]pyrimidin-6-yl]-6-methylpiperazin-2-one
N-{(1R)-1-[3-(difluoromethyl)-2-fluorophenyl]ethyl}-6-(3,3-dimethylpiperazin-1-yl)-2-methylpyrido[3,4-d]pyrimidin-4-amine
N-{(1R)-1-[3-(difluoromethyl)-2-fluorophenyl]ethyl}-2-methyl-6-(4-methyl-1,4-diazepan-1-yl)pyrido[3,4-d]pyrimidin-4-amine
N-{(1R)-1-[3-(difluoromethyl)-2-fluorophenyl]ethyl}-6-(4-ethylpiperazin-1-yl)-2-methylpyrido[3,4-d]pyrimidin-4-amine
N-{(1R)-1-[3-(difluoromethyl)-2-fluorophenyl]ethyl}-6-[(3S)-3-(dimethylamino)pyrrolidin-1-yl]-2-methylpyrido[3,4-d]pyrimidin-4-amine
N-{(1R)-1-[3-(difluoromethyl)-2-fluorophenyl]ethyl}-6-[(3R)-3-(dimethylamino)pyrrolidin-1-yl]-2-methylpyrido[3,4-d]pyrimidin-4-amine
{1-[4-({(1R)-1-[3-(difluoromethyl)-2-fluorophenyl]ethyl}amino)-2-methylpyrido[3,4-d]pyrimidin-6-yl]piperidin-4-yl}methanol
N⁴-{(1R)-1-[3-(difluoromethyl)-2-fluorophenyl]ethyl}-N⁶,2-dimethyl-N⁶-(oxan-4-yl)pyrido[3,4-d]pyrimidine-4,6-diamine
4-{[4-({(1R)-1-[3-(difluoromethyl)-2-fluorophenyl]ethyl}amino)-2-methylpyrido[3,4-d]pyrimidin-6-yl]amino}cyclohexan-1-ol (mixture of stereoisomers)
(1RS,4SR,5RS)-2-[4-({(1R)-1-[3-(difluoromethyl)-2-fluorophenyl]ethyl}amino)-2-methylpyrido[3,4-d]pyrimidin-6-yl]-2-azabicyclo[2.2.1]heptane-5-carbonitrile (mixture of stereoisomers)
N²-[4-({(1R)-1-[3-(difluoromethyl)-2-fluorophenyl]ethyl}amino)-2-methylpyrido[3,4-d]pyrimidin-6-yl]-N,N,N²-trimethylglycinamide
N-{(1R)-1-[3-(difluoromethyl)-2-fluorophenyl]ethyl}-6-(6,7-dihydropyrazolo[1,5-a]pyrazin-5(4H)-yl)-2-methylpyrido[3,4-d]pyrimidin-4-amine
N-{(1R)-1-[3-(difluoromethyl)-2-fluorophenyl]ethyl}-6-(5,6-dihydroimidazo[1,5-a]pyrazin-7(8H)-yl)-2-methylpyrido[3,4-d]pyrimidin-4-amine
N-{(1R)-1-[3-(difluoromethyl)-2-fluorophenyl]ethyl}-6-(5,6-dihydroimidazo[1,2-a]pyrazin-7(8H)-yl)-2-methylpyrido[3,4-d]pyrimidin-4-amine
N-{(1R)-1-[3-(difluoromethyl)-2-fluorophenyl]ethyl}-2-methyl-6-(1-methyl-4,6-dihydropyrrolo[3,4-c]pyrazol-5(1H)-yl)pyrido[3,4-d]pyrimidin-4-amine
N-{(1R)-1-[3-(difluoromethyl)-2-fluorophenyl]ethyl}-6-(5,6-dihydro[1,2,4]triazolo[1,5-a]pyrazin-7(8H)-yl)-2-methylpyrido[3,4-d]pyrimidin-4-amine
1-[4-({(1R)-1-[3-(difluoromethyl)-2-fluorophenyl]ethyl}amino)-2-methylpyrido[3,4-d]pyrimidin-6-yl]-4-methylpiperidine-4-carbonitrile
{4-[4-({(1R)-1-[3-(difluoromethyl)-2-fluorophenyl]ethyl}amino)-2-methylpyrido[3,4-d]pyrimidin-6-yl]piperazin-1-yl}acetonitrile
2-[4-({(1R)-1-[3-(difluoromethyl)-2-fluorophenyl]ethyl}amino)-2-methylpyrido[3,4-d]pyrimidin-6-yl]-2,6-diazaspiro[3.4]octan-5-one
2-[4-({(1R)-1-[3-(difluoromethyl)-2-fluorophenyl]ethyl}amino)-2-methylpyrido[3,4-d]pyrimidin-6-yl]-2,6-diazaspiro[3.4]octan-7-one
N-{(1R)-1-[3-(difluoromethyl)-2-fluorophenyl]ethyl}-2-methyl-6-[(3aS,6aS)-1-methylhexahydropyrrolo[3,4-b]pyrrol-5(1H)-yl]pyrido[3,4-d]pyrimidin-4-amine
N-{(1R)-1-[3-(difluoromethyl)-2-fluorophenyl]ethyl}-2-methyl-6-[(3aRS,6aSR)-5-methylhexahydropyrrolo[3,4-c]pyrrol-2(1H)-yl]pyrido[3,4-d]pyrimidin-4-amine (mixture of stereoisomers)
N-{(1R)-1-[3-(difluoromethyl)-2-fluorophenyl]ethyl}-2-methyl-6-[(3aR,6aR)-1-methylhexahydropyrrolo[3,4-b]pyrrol-5(1H)-yl]pyrido[3,4-d]pyrimidin-4-amine
N-{(1R)-1-[3-(difluoromethyl)-2-fluorophenyl]ethyl}-6-[(8aS)-hexahydropyrrolo[1,2-a]pyrazin-2(1H)-yl]-2-methylpyrido[3,4-d]pyrimidin-4-amine
N-{(1R)-1-[3-(difluoromethyl)-2-fluorophenyl]ethyl}-6-[(8aR)-hexahydropyrrolo[1,2-a]pyrazin-2(1H)-yl]-2-methylpyrido[3,4-d]pyrimidin-4-amine
N-{(1R)-1-[3-(difluoromethyl)-2-fluorophenyl]ethyl}-2-methyl-6-(6-methyl-2,6-diazaspiro[3.4]octan-2-yl)pyrido[3,4-d]pyrimidin-4-amine
6-(4-cyclopropylpiperazin-1-yl)-N-{(1R)-1-[3-(difluoromethyl)-2-fluorophenyl]ethyl}-2-methylpyrido[3,4-d]pyrimidin-4-amine
N-{(1R)-1-[3-(difluoromethyl)-2-fluorophenyl]ethyl}-2-methyl-6-(2-oxa-6-azaspiro[3.5]nonan-6-yl)pyrido[3,4-d]pyrimidin-4-amine
N-{(1R)-1-[3-(difluoromethyl)-2-fluorophenyl]ethyl}-2-methyl-6-(2-oxa-7-azaspiro[3.5]nonan-7-yl)pyrido[3,4-d]pyrimidin-4-amine
(3RS)-1-[4-({(1R)-1-[3-(difluoromethyl)-2-fluorophenyl]ethyl}amino)-2-methylpyrido[3,4-d]pyrimidin-6-yl]-3-methylpyrrolidine-3-carboxamide
1-[4-({(1R)-1-[3-(difluoromethyl)-2-fluorophenyl]ethyl}amino)-2-methylpyrido[3,4-d]pyrimidin-6-yl]piperidine-4-carboxamide
1-{4-[4-({(1R)-1-[3-(difluoromethyl)-2-fluorophenyl]ethyl}amino)-2-methylpyrido[3,4-d]pyrimidin-6-yl]piperazin-1-yl}ethan-1-one
(3R)-1-[4-({(1R)-1-[3-(difluoromethyl)-2-fluorophenyl]ethyl}amino)-2-methylpyrido[3,4-d]pyrimidin-6-yl]piperidine-3-carboxamide
(3S)-1-[4-({(1R)-1-[3-(difluoromethyl)-2-fluorophenyl]ethyl}amino)-2-methylpyrido[3,4-d]pyrimidin-6-yl]piperidine-3-carboxamide
N-{(1R)-1-[3-(difluoromethyl)-2-fluorophenyl]ethyl}-2-methyl-6-[(cis)-3,4,5-trimethylpiperazin-1-yl]pyrido[3,4-d]pyrimidin-4-amine (mixture of stereoisomers)
N-{(1R)-1-[3-(difluoromethyl)-2-fluorophenyl]ethyl}-2-methyl-6-[(3R,5R)-3,4,5-trimethylpiperazin-1-yl]pyrido[3,4-d]pyrimidin-4-amine
N-{(1R)-1-[3-(difluoromethyl)-2-fluorophenyl]ethyl}-2-methyl-6-[(3S,5S)-3,4,5-trimethylpiperazin-1-yl]pyrido[3,4-d]pyrimidin-4-amine
N-{(1R)-1-[3-(difluoromethyl)-2-fluorophenyl]ethyl}-6-[3-(dimethylamino)piperidin-1-yl]-2-methylpyrido[3,4-d]pyrimidin-4-amine (mixture of stereoisomers)
N-{(1R)-1-[3-(difluoromethyl)-2-fluorophenyl]ethyl}-6-[4-(dimethylamino)piperidin-1-yl]-2-methylpyrido[3,4-d]pyrimidin-4-amine
1-[4-({(1R)-1-[3-(difluoromethyl)-2-fluorophenyl]ethyl}amino)-2-methylpyrido[3,4-d]pyrimidin-6-yl]-3-methylpyrrolidine-3-carboxylic acid (mixture of stereoisomers)
4-{[4-({(1R)-1-[3-(difluoromethyl)-2-fluorophenyl]ethyl}amino)-2-methylpyrido[3,4-d]pyrimidin-6-yl]amino}-1-methylcyclohexan-1-ol (mixture of stereoisomers)
2-{4-[4-({(1R)-1-[3-(difluoromethyl)-2-fluorophenyl]ethyl}amino)-2-methylpyrido[3,4-d]pyrimidin-6-yl]piperazin-1-yl}ethan-1-ol
1-[4-({(1R)-1-[3-(difluoromethyl)-2-fluorophenyl]ethyl}amino)-2-methylpyrido[3,4-d]pyrimidin-6-yl]-3-(2-hydroxyethyl)pyrrolidin-3-ol (mixture of stereoisomers)
N-{(1R)-1-[3-(difluoromethyl)-2-fluorophenyl]ethyl}-2-methyl-6-(3-methyl-5,6-dihydro[1,2,4]triazolo[4,3-a]pyrazin-7(8H)-yl)pyrido[3,4-d]pyrimidin-4-amine
2-[4-({(1R)-1-[3-(difluoromethyl)-2-fluorophenyl]ethyl}amino)-2-methylpyrido[3,4-d]pyrimidin-6-yl]hexahydropyrrolo[1,2-a]pyrazin-6(2H)-one (mixture of stereoisomers)
(5RS)-7-[4-({(1R)-1-[3-(difluoromethyl)-2-fluorophenyl]ethyl}amino)-2-methylpyrido[3,4-d]pyrimidin-6-yl]-2,7-diazaspiro[4.4]nonan-3-one (mixture of stereoisomers)
6-[[1,3′-bipyrrolidin]-1′-yl]-N-{(1R)-1-[3-(difluoromethyl)-2-fluorophenyl]ethyl}-2-methylpyrido[3,4-d]pyrimidin-4-amine (mixture of stereoisomers)
7-[4-({(1R)-1-[3-(difluoromethyl)-2-fluorophenyl]ethyl}amino)-2-methylpyrido[3,4-d]pyrimidin-6-yl]hexahydro-3H-[1,3]oxazolo[3,4-a]pyrazin-3-one (mixture of stereoisomers)
1-[4-({(1R)-1-[3-(difluoromethyl)-2-fluorophenyl]ethyl}amino)-2-methylpyrido[3,4-d]pyrimidin-6-yl]-4-methyl-1,4-diazepane-2,3-dione
1-{4-[4-({(1R)-1-[3-(difluoromethyl)-2-fluorophenyl]ethyl}amino)-2-methylpyrido[3,4-d]pyrimidin-6-yl]-1,4-diazepan-1-yl}ethan-1-one
N-{(3RS)-1-[4-({(1R)-1-[3-(difluoromethyl)-2-fluorophenyl]ethyl}amino)-2-methylpyrido[3,4-d]pyrimidin-6-yl]pyrrolidin-3-yl}-N-methylacetamide (mixture of stereoisomers)
N-{1-[4-({(1R)-1-[3-(difluoromethyl)-2-fluorophenyl]ethyl}amino)-2-methylpyrido[3,4-d]pyrimidin-6-yl]piperidin-4-yl}acetamide
(3RS)-1-[4-({(1R)-1-[3-(difluoromethyl)-2-fluorophenyl]ethyl}amino)-2-methylpyrido[3,4-d]pyrimidin-6-yl]-N-methylpiperidine-3-carboxamide (mixture of stereoisomers)
2-{1-[4-({(1R)-1-[3-(difluoromethyl)-2-fluorophenyl]ethyl}amino)-2-methylpyrido[3,4-d]pyrimidin-6-yl]piperidin-4-yl}propan-2-ol
(2R)-4-[4-({(1R)-1-[3-(difluoromethyl)-2-fluorophenyl]ethyl}amino)-2-methylpyrido[3,4-d]pyrimidin-6-yl]-6-oxopiperazine-2-carboxylic acid
N-{(1R)-1-[3-(difluoromethyl)-2-fluorophenyl]ethyl}-6-[4-(2-methoxyethyl)piperazin-1-yl]-2-methylpyrido[3,4-d]pyrimidin-4-amine
5-[4-({(1R)-1-[3-(difluoromethyl)-2-fluorophenyl]ethyl}amino)-2-methylpyrido[3,4-d]pyrimidin-6-yl]-4,5,6,7-tetrahydropyrazolo[1,5-a]pyrazine-2-carbonitrile
6-[4-(2,2-difluoroethyl)piperazin-1-yl]-N-{(1R)-1-[3-(difluoromethyl)-2-fluorophenyl]ethyl}-2-methylpyrido[3,4-d]pyrimidin-4-amine
1-[5-[4-({(1R)-1-[3-(difluoromethyl)-2-fluorophenyl]ethyl}amino)-2-methylpyrido[3,4-d]pyrimidin-6-yl]hexahydropyrrolo[3,4-c]pyrrol-2(1H)-yl]ethan-1-one (mixture of stereoisomers)
N-{(1R)-1-[3-(difluoromethyl)-2-fluorophenyl]ethyl}-2-methyl-6-[3-(piperidin-1-yl)pyrrolidin-1-yl]pyrido[3,4-d]pyrimidin-4-amine (mixture of stereoisomers)
N-{(1R)-1-[3-(difluoromethyl)-2-fluorophenyl]ethyl}-2-methyl-6-[3-(morpholin-4-yl)pyrrolidin-1-yl]pyrido[3,4-d]pyrimidin-4-amine (mixture of stereoisomers)
6-[7,7-difluorohexahydropyrrolo[1,2-a]pyrazin-2(1H)-yl]-N-{(1R)-1-[3-(difluoromethyl)-2-fluorophenyl]ethyl}-2-methylpyrido[3,4-d]pyrimidin-4-amine (mixture of stereoisomers)
(3RS)-1-[4-({(1R)-1-[3-(difluoromethyl)-2-fluorophenyl]ethyl}amino)-2-methylpyrido[3,4-d]pyrimidin-6-yl]piperidine-3-sulfonamide
N-{(1R)-1-[3-(difluoromethyl)-2-fluorophenyl]ethyl}-2-methyl-6-[4-(2,2,2-trifluoroethyl)piperazin-1-yl]pyrido[3,4-d]pyrimidin-4-amine
tert-butyl {(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}carbamate
tert-butyl {3-[4-({(1R)-1-[3-(difluoromethyl)-2-fluorophenyl]ethyl}amino)-2-methylpyrido[3,4-d]pyrimidin-6-yl]-3-azabicyclo[3.1.0]hexan-1-yl}carbamate (mixture of stereoisomers)
tert-butyl {1-[4-({(1R)-1-[3-(difluoromethyl)-2-fluorophenyl]ethyl}amino)-2-methylpyrido[3,4-d]pyrimidin-6-yl]-4-fluoropyrrolidin-3-yl}carbamate (mixture of stereoisomers)
tert-butyl 6-[4-({(1R)-1-[3-(difluoromethyl)-2-fluorophenyl]ethyl}amino)-2-methylpyrido[3,4-d]pyrimidin-6-yl]-2,6-diazaspiro[3.4]octane-2-carboxylate
tert-butyl 2-[4-({(1R)-1-[3-(difluoromethyl)-2-fluorophenyl]ethyl}amino)-2-methylpyrido[3,4-d]pyrimidin-6-yl]-2,7-diazaspiro[3.5]nonane-7-carboxylate
tert-butyl 7-[4-({(1R)-1-[3-(difluoromethyl)-2-fluorophenyl]ethyl}amino)-2-methylpyrido[3,4-d]pyrimidin-6-yl]-2,7-diazaspiro[3.5]nonane-2-carboxylate
N-{(1R)-1-[3-(difluoromethyl)-2-fluorophenyl]ethyl}-2-methyl-6-(6-methyl-2,6-diazaspiro[3.4]octan-2-yl)pyrido[3,4-d]pyrimidin-4-amine
tert-butyl 2-[4-({(1R)-1-[3-(difluoromethyl)-2-fluorophenyl]ethyl}amino)-2-methylpyrido[3,4-d]pyrimidin-6-yl]-2,6-diazaspiro[3.4]octane-6-carboxylate
methyl 4-(2-{4-[4-({(1R)-1-[3-(difluoromethyl)-2-fluorophenyl]ethyl}amino)-2-methylpyrido[3,4-d]pyrimidin-6-yl]piperazin-1-yl}ethoxy)benzoate
4-(2-{4-[4-({(1R)-1-[3-(difluoromethyl)-2-fluorophenyl]ethyl}amino)-2-methylpyrido[3,4-d]pyrimidin-6-yl]piperazin-1-yl}ethoxy)benzoic acid
6-(methanesulfonyl)-2-methyl-N-{(1R)-1-[3-(trifluoromethyl)phenyl]ethyl}pyrido[3,4-d]pyrimidin-4-amine
6-[(3R)-3-aminopyrrolidin-1-yl]-N-{(1R)-1-[3-(difluoromethyl)-2-fluorophenyl]ethyl}-2-methylpyrido[3,4-d]pyrimidin-4-amine hydrochloride salt
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}cyclopropanecarboxamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-2,2-difluoroacetamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-2-methoxyacetamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}oxetane-3-carboxamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-1-methylazetidine-3-carboxamide
methyl {(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}carbamate
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}methanesulfonamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}cyclopropanesulfonamide
cyclopropyl{4-[4-({(1R)-1-[3-(difluoromethyl)-2-fluorophenyl]ethyl}amino)-2-methylpyrido[3,4-d]pyrimidin-6-yl]piperazin-1-yl}methanone
1-{4-[4-({(1R)-1-[3-(difluoromethyl)-2-fluorophenyl]ethyl}amino)-2-methylpyrido[3,4-d]pyrimidin-6-yl]piperazin-1-yl}-2-methoxyethan-1-one
1-{4-[4-({(1R)-1-[3-(difluoromethyl)-2-fluorophenyl]ethyl}amino)-2-methylpyrido[3,4-d]pyrimidin-6-yl]piperazin-1-yl}-2,2-difluoroethan-1-one
{4-[4-({(1R)-1-[3-(difluoromethyl)-2-fluorophenyl]ethyl}amino)-2-methylpyrido[3,4-d]pyrimidin-6-yl]piperazin-1-yl}(oxetan-3-yl)methanone
1-{4-[4-({(1R)-1-[3-(difluoromethyl)-2-fluorophenyl]ethyl}amino)-2-methylpyrido[3,4-d]pyrimidin-6-yl]piperazin-1-yl}-2-(dimethylamino)ethan-1-one
{4-[4-({(1R)-1-[3-(difluoromethyl)-2-fluorophenyl]ethyl}amino)-2-methylpyrido[3,4-d]pyrimidin-6-yl]piperazin-1-yl}(1-fluorocyclopropyl)methanone
1-{4-[4-({(1R)-1-[3-(difluoromethyl)-2-fluorophenyl]ethyl}amino)-2-methylpyrido[3,4-d]pyrimidin-6-yl]piperazin-1-yl}-2,2-difluoropropan-1-one
1-{4-[4-({(1R)-1-[3-(difluoromethyl)-2-fluorophenyl]ethyl}amino)-2-methylpyrido[3,4-d]pyrimidin-6-yl]piperazine-1-carbonyl}cyclopropane-1-carbonitrile
methyl 10-{4-[4-({(1R)-1-[3-(difluoromethyl)-2-fluorophenyl]ethyl}amino)-2-methylpyrido[3,4-d]pyrimidin-6-yl]piperazin-1-yl}-10-oxodecanoate
10-{4-[4-({(1R)-1-[3-(difluoromethyl)-2-fluorophenyl]ethyl}amino)-2-methylpyrido[3,4-d]pyrimidin-6-yl]piperazin-1-yl}-10-oxodecanoic acid
4-[4-({(1R)-1-[3-(difluoromethyl)-2-fluorophenyl]ethyl}amino)-2-methylpyrido[3,4-d]pyrimidin-6-yl]-N,N-dimethylpiperazine-1-carboxamide
N-{(1R)-1-[3-(difluoromethyl)-2-fluorophenyl]ethyl}-6-[4-(methanesulfonyl)piperazin-1-yl]-2-methylpyrido[3,4-d]pyrimidin-4-amine
2-amino-1-{4-[4-({(1R)-1-[3-(difluoromethyl)-2-fluorophenyl]ethyl}amino)-2-methylpyrido[3,4-d]pyrimidin-6-yl]piperazin-1-yl}ethan-1-one
1-{4-[4-({(1R)-1-[3-(difluoromethyl)-2-fluorophenyl]ethyl}amino)-2-methylpyrido[3,4-d]pyrimidin-6-yl]piperazin-1-yl}-2-(methylamino)ethan-1-one
3-amino-1-{4-[4-({(1R)-1-[3-(difluoromethyl)-2-fluorophenyl]ethyl}amino)-2-methylpyrido[3,4-d]pyrimidin-6-yl]piperazin-1-yl}propan-1-one
1-{4-[4-({(1R)-1-[3-(difluoromethyl)-2-fluorophenyl]ethyl}amino)-2-methylpyrido[3,4-d]pyrimidin-6-yl]piperazin-1-yl}-3-(methylamino)propan-1-one
6-[(3R)-3-(dimethylamino)pyrrolidin-1-yl]-2-methyl-N-{(1R)-1-[3-(trifluoromethyl)phenyl]ethyl}pyrido[3,4-d]pyrimidin-4-amine
2-[2-methyl-4-({(1R)-1-[3-(trifluoromethyl)phenyl]ethyl}amino)pyrido[3,4-d]pyrimidin-6-yl]-2,6-diazaspiro[3.4]octan-7-one
1-{4-[2-methyl-4-({(1R)-1-[3-(trifluoromethyl)phenyl]ethyl}amino)pyrido[3,4-d]pyrimidin-6-yl]piperazin-1-yl}ethan-1-one
2-methyl-6-(4-methylpiperazin-1-yl)-N-{(1R)-1-[3-(trifluoromethyl)phenyl]ethyl}pyrido[3,4-d]pyrimidin-4-amine
6-fluoro-2-methyl-N-{(1R)-1-[2-methyl-3-(trifluoromethyl)phenyl]ethyl}pyrido[3,4-d]pyrimidin-4-amine
2-methyl-6-(4-methylpiperazin-1-yl)-N-{(1R)-1-[2-methyl-3-(trifluoromethyl)phenyl]ethyl}pyrido[3,4-d]pyrimidin-4-amine
2-[2-methyl-4-({(1R)-1-[2-methyl-3-(trifluoromethyl)phenyl]ethyl}amino)pyrido[3,4-d]pyrimidin-6-yl]-2,6-diazaspiro[3.4]octan-7-one
6-[(3R)-3-(dimethylamino)pyrrolidin-1-yl]-2-methyl-N-{(1R)-1-[2-methyl-3-(trifluoromethyl)phenyl]ethyl}pyrido[3,4-d]pyrimidin-4-amine
1-{4-[2-methyl-4-({(1R)-1-[2-methyl-3-(trifluoromethyl)phenyl]ethyl}amino)pyrido[3,4-d]pyrimidin-6-yl]piperazin-1-yl}ethan-1-one
2-methyl-N-{(1R)-1-[2-methyl-3-(trifluoromethyl)phenyl]ethyl}-6-(1-oxa-6-azaspiro[3.3]heptan-6-yl)pyrido[3,4-d]pyrimidin-4-amine
6-fluoro-2,8-dimethyl-N-{(1R)-1-[3-(trifluoromethyl)phenyl]ethyl}pyrido[3,4-d]pyrimidin-4-amine
1-{4-[2,8-dimethyl-4-({(1R)-1-[3-(trifluoromethyl)phenyl]ethyl}amino)pyrido[3,4-d]pyrimidin-6-yl]piperazin-1-yl}ethan-1-one
2,8-dimethyl-6-(4-methylpiperazin-1-yl)-N-{(1R)-1-[3-(trifluoromethyl)phenyl]ethyl}pyrido[3,4-d]pyrimidin-4-amine
6-[(3R)-3-(dimethylamino)pyrrolidin-1-yl]-2,8-dimethyl-N-{(1R)-1-[3-(trifluoromethyl)phenyl]ethyl}pyrido[3,4-d]pyrimidin-4-amine
2-[2,8-dimethyl-4-({(1R)-1-[3-(trifluoromethyl)phenyl]ethyl}amino)pyrido[3,4-d]pyrimidin-6-yl]-2,6-diazaspiro[3.4]octan-7-one
6-fluoro-2,8-dimethyl-N-{(1R)-1-[2-methyl-3-(trifluoromethyl)phenyl]ethyl}pyrido[3,4-d]pyrimidin-4-amine
1-{4-[2,8-dimethyl-4-({(1R)-1-[2-methyl-3-(trifluoromethyl)phenyl]ethyl}amino)pyrido[3,4-d]pyrimidin-6-yl]piperazin-1-yl}ethan-1-one
2-[2,8-dimethyl-4-({(1R)-1-[2-methyl-3-(trifluoromethyl)phenyl]ethyl}amino)pyrido[3,4-d]pyrimidin-6-yl]-2,6-diazaspiro[3.4]octan-7-one
2,8-dimethyl-6-(4-methylpiperazin-1-yl)-N-{(1R)-1-[2-methyl-3-(trifluoromethyl)phenyl]ethyl}pyrido[3,4-d]pyrimidin-4-amine
6-[(3R)-3-(dimethylamino)pyrrolidin-1-yl]-2,8-dimethyl-N-{(1R)-1-[2-methyl-3-(trifluoromethyl)phenyl]ethyl}pyrido[3,4-d]pyrimidin-4-amine
N-{(3R)-1-[2,8-dimethyl-4-({(1R)-1-[2-methyl-3-(trifluoromethyl)phenyl]ethyl}amino)pyrido[3,4-d]pyrimidin-6-yl]pyrrolidin-3-yl}acetamide
N-{(3S)-1-[2,8-dimethyl-4-({(1R)-1-[2-methyl-3-(trifluoromethyl)phenyl]ethyl}amino)pyrido[3,4-d]pyrimidin-6-yl]pyrrolidin-3-yl}acetamide
6-chloro-2-methyl-N-{(1R)-1-[3-(trifluoromethyl)phenyl]ethyl}pyrido[3,4-d]pyrimidin-4-amine
2-methyl-6-(1-methyl-1H-pyrazol-4-yl)-N-{(1R)-1-[3-(trifluoromethyl)phenyl]ethyl}pyrido[3,4-d]pyrimidin-4-amine
6-(4,5-dihydrofuran-2-yl)-2-methyl-N-{(1R)-1-[3-(trifluoromethyl)phenyl]ethyl}pyrido[3,4-d]pyrimidin-4-amine
6-(2,5-dihydrofuran-3-yl)-2-methyl-N-{(1R)-1-[3-(trifluoromethyl)phenyl]ethyl}pyrido[3,4-d]pyrimidin-4-amine
6-(3,6-dihydro-2H-pyran-4-yl)-2-methyl-N-{(1R)-1-[3-(trifluoromethyl)phenyl]ethyl}pyrido[3,4-d]pyrimidin-4-amine
6-(5,6-dihydro-2H-pyran-3-yl)-2-methyl-N-{(1R)-1-[3-(trifluoromethyl)phenyl]ethyl}pyrido[3,4-d]pyrimidin-4-amine
2-methyl-6-(1-methyl-1,2,3,6-tetrahydropyridin-4-yl)-N-{(1R)-1-[3-(trifluoromethyl)phenyl]ethyl}pyrido[3,4-d]pyrimidin-4-amine
2-methyl-6-[(3RS)-oxolan-3-yl]-N-{(1R)-1-[3-(trifluoromethyl)phenyl]ethyl}pyrido[3,4-d]pyrimidin-4-amine amine (mixture of stereoisomers)
2-methyl-6-(oxan-4-yl)-N-{(1R)-1-[3-(trifluoromethyl)phenyl]ethyl}pyrido[3,4-d]pyrimidin-4-amine
2-methyl-6-[(3RS)-oxan-3-yl]-N-{(1R)-1-[3-(trifluoromethyl)phenyl]ethyl}pyrido[3,4-d]pyrimidin-4-amine (mixture of stereoisomers)
2-methyl-6-(1-methylpiperidin-4-yl)-N-{(1R)-1-[3-(trifluoromethyl)phenyl]ethyl}pyrido[3,4-d]pyrimidin-4-amine
methyl 2-methyl-4-({(1R)-1-[3-(trifluoromethyl)phenyl]ethyl}amino)pyrido[3,4-d]pyrimidine-6-carboxylate
2-methyl-4-({(1R)-1-[3-(trifluoromethyl)phenyl]ethyl}amino)pyrido[3,4-d]pyrimidine-6-carboxamide
N,2-dimethyl-4-({(1R)-1-[3-(trifluoromethyl)phenyl]ethyl}amino)pyrido[3,4-d]pyrimidine-6-carboxamide
1-[4-({(1R)-1-[3-(difluoromethyl)-2-methylphenyl]ethyl}amino)-2-methylpyrido[3,4-d]pyrimidin-6-yl]piperidine-4-carbonitrile
N-{(1R)-1-[3-(difluoromethyl)-2-fluorophenyl]ethyl}-6-[(2S)-2,4-dimethylpiperazin-1-yl]-2-methylpyrido[3,4-d]pyrimidin-4-amine
{1-[4-({(1R)-1-[3-(difluoromethyl)-2-fluorophenyl]ethyl}amino)-2-methylpyrido[3,4-d]pyrimidin-6-yl]-4-methylpiperazin-2-yl}methanol (mixture of stereoisomers)
N-{(1R)-1-[3-(difluoromethyl)-2-fluorophenyl]ethyl}-2-methyl-6-[2-(trifluoromethyl)-5,6-dihydroimidazo[1,2-a]pyrazin-7(8H)-yl]pyrido[3,4-d]pyrimidin-4-amine
N-{(1R)-1-[3-(difluoromethyl)-2-fluorophenyl]ethyl}-2-methyl-6-[2-(trifluoromethyl)-5,6-dihydro[1,2,4]triazolo[1,5-a]pyrazin-7(8H)-yl]pyrido[3,4-d]pyrimidin-4-amine
6-(cyclobutyloxy)-N-{(1R)-1-[3-(difluoromethyl)-2-fluorophenyl]ethyl}-2-methylpyrido[3,4-d]pyrimidin-4-amine
6-butoxy-N-{(1R)-1-[3-(difluoromethyl)-2-fluorophenyl]ethyl}-2-methylpyrido[3,4-d]pyrimidin-4-amine
N-{(1R)-1-[3-(difluoromethyl)-2-fluorophenyl]ethyl}-2-methyl-6-[2-(methylamino)ethoxy]pyrido[3,4-d]pyrimidin-4-amine
N-[(1R)-1-{3-(difluoromethyl)-2-[2-(methylamino)ethoxy]phenyl}ethyl]-2-methyl-6-[2-(methylamino)ethoxy]pyrido[3,4-d]pyrimidin-4-amine
N-{(1R)-1-[3-(difluoromethyl)-2-fluorophenyl]ethyl}-2-methyl-6-[(oxetan-3-yl)oxy]pyrido[3,4-d]pyrimidin-4-amine
tert-butyl 3-{[4-({(1R)-1-[3-(difluoromethyl)-2-fluorophenyl]ethyl}amino)-2-methylpyrido[3,4-d]pyrimidin-6-yl]oxy}azetidine-1-carboxylate
N-{(1R)-1-[3-(difluoromethyl)-2-fluorophenyl]ethyl}-2-methyl-6-{[(3R)-oxolan-3-yl]oxy}pyrido[3,4-d]pyrimidin-4-amine
N-{(1R)-1-[3-(difluoromethyl)-2-{[(3R)-oxolan-3-yl]oxy}phenyl]ethyl}-2-methyl-6-{[(3R)-oxolan-3-yl]oxy}pyrido[3,4-d]pyrimidin-4-amine
N-{(1R)-1-[3-(difluoromethyl)-2-fluorophenyl]ethyl}-2-methyl-6-{[(3S)-oxolan-3-yl]oxy}pyrido[3,4-d]pyrimidin-4-amine
N-{(1R)-1-[3-(difluoromethyl)-2-{[(3S)-oxolan-3-yl]oxy}phenyl]ethyl}-2-methyl-6-{[(3S)-oxolan-3-yl]oxy}pyrido[3,4-d]pyrimidin-4-amine
N-{(1R)-1-[3-(difluoromethyl)-2-{[(3S)-1-methylpyrrolidin-3-yl]oxy}phenyl]ethyl}-2-methyl-6-{[(3S)-1-methylpyrrolidin-3-yl]oxy}pyrido[3,4-d]pyrimidin-4-amine
6-[(azetidin-3-yl)oxy]-N-{(1R)-1-[3-(difluoromethyl)-2-fluorophenyl]ethyl}-2-methylpyrido[3,4-d]pyrimidin-4-amine hydrochloride
tert-butyl {(3-trans)-1-[4-({(1R)-1-[3-(difluoromethyl)-2-fluorophenyl]ethyl}amino)-2-methylpyrido[3,4-d]pyrimidin-6-yl]-4-fluoropyrrolidin-3-yl}carbamate (mixture of stereoisomers)
6-[(trans)-3-amino-4-fluoropyrrolidin-1-yl]-N-{(1R)-1-[3-(difluoromethyl)-2-fluorophenyl]ethyl}-2-methylpyrido[3,4-d]pyrimidin-4-amine hydrochloride (mixture of stereoisomers)
tert-butyl {(cis)-1-[4-({(1R)-1-[3-(difluoromethyl)-2-fluorophenyl]ethyl}amino)-2-methylpyrido[3,4-d]pyrimidin-6-yl]-4-fluoropyrrolidin-3-yl}carbamate (mixture of stereoisomers)
6-[(cis)-3-amino-4-fluoropyrrolidin-1-yl]-N-{(1R)-1-[3-(difluoromethyl)-2-fluorophenyl]ethyl}-2-methylpyrido[3,4-d]pyrimidin-4-amine hydrochloride (mixture of stereoisomers)

or a stereoisomer, a tautomer, an N-oxide, a hydrate, a solvate, or a salt thereof, or a mixture of same.

9. A compound of general formula (1) according to any one of claims 1 to 8 for use in the treatment or prophylaxis of a disease.

10. A pharmaceutical composition comprising a compound of general formula (1) according to any one of claims 1 to 8 and one or more pharmaceutically acceptable excipients.

11. A pharmaceutical combination comprising:
one or more first active ingredients, in particular compounds of general formula (1) according to any one of claims 1 to 8, and
one or more further active ingredients, in particular anti-hyperproliferative and/or anti-cancer agents.

12. Use of a compound of general formula (1) according to any one of claims 1 to 8 for the treatment or prophylaxis of a disease.

13. Use of a compound of general formula (1) according to any one of claims 1 to 8 for the preparation of a medicament for the treatment or prophylaxis of a disease.

14. Use according to claim 9, 12 or 13, wherein the disease is a hyperproliferative disorder, such as cancer, for example.

15. Use of SOS1 Inhibitors for the treatment or prophylaxis of a disease, especially for the treatment or prophylaxis of cancer.

The present invention covers any sub-combination within any embodiment or aspect of the present invention of compounds of general formula (I), supra.

The present invention covers any sub-combination within any embodiment or aspect of the present invention of intermediate compounds of general formula (II).The present invention covers the compounds of general formula (I) which are disclosed in the Example Section of this text, infra.

Synthesis of Compounds (Overview)

The compounds of the present invention can be prepared as described in the following section. The schemes and the procedures described below illustrate general synthetic routes to the compounds of general formula (I) of the invention and are not intended to be limiting. It is clear to the person skilled in the art that the order of transformations as exemplified in the schemes can be modified in various ways. The order of transformations exemplified in the schemes is therefore not intended to be limiting. In addition, interconversion of any of the substituents can be achieved before and/or after the exemplified transformations. These modifications can be such as the introduction of protecting groups, cleavage of protecting groups, exchange, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example P. G. M. Wuts and T. W. Greene in “Protective Groups in Organic Synthesis”, 4′″ edition, Wiley 2006). Specific examples are described in the subsequent paragraphs. Further, it is possible that two or more successive steps may be performed without work-up being performed between said steps, e.g. a “one-pot” reaction, as is well-known to the person skilled in the art.

The syntheses of the compounds of the present invention are preferably carried out according to the general synthetic sequence, shown in schemes 1-7.

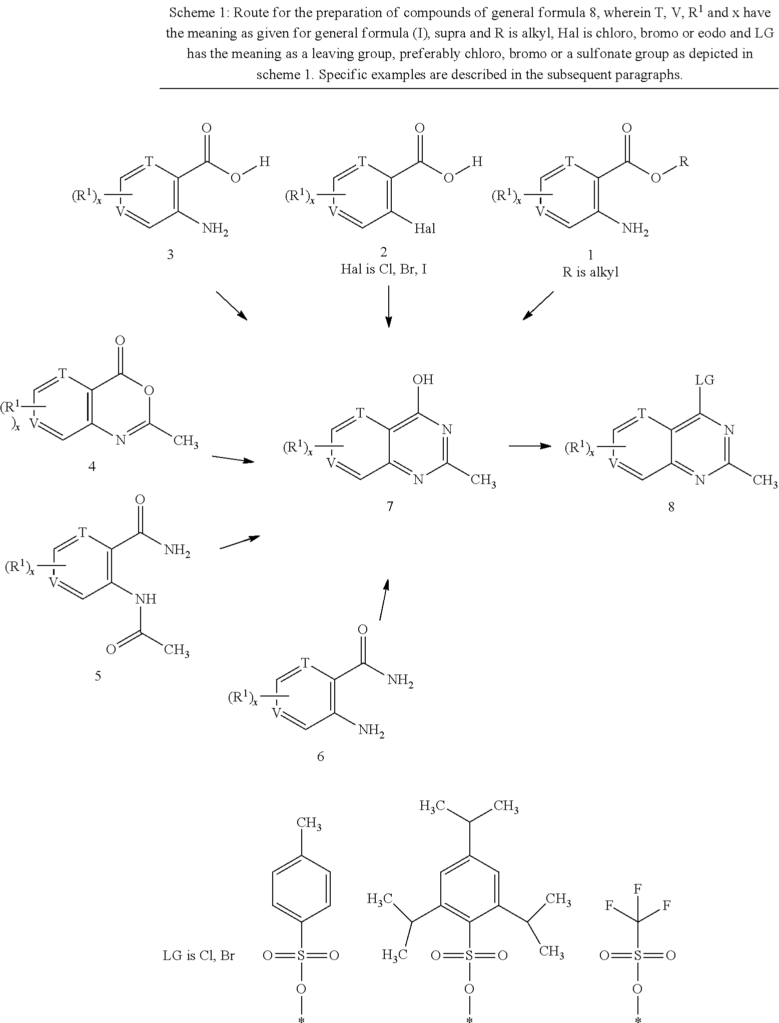

Step 1→7 (Scheme 1)

Azaquinazoline Formation

In the first step (scheme 1) amino acid ester derivative 1 (which is commercially available or described in the literature) can be converted to the corresponding azaquinazoline 7 in analogy to literature procedures. Typically acetonitrile and hydrochloric acid in organic solvent such as for example 1,4-dioxane at elevated temperatures is used. For example see ACS Medicinal Chemistry Letters, 2013, vol. 4, #9 p. 846-851; Journal of Medicinal Chemistry, 2009, vol. 52, #8 p. 2341-2351 or WO2015/54572 and references therein.

Step 2→7 (Scheme 1)

Azaquinazoline Formation

Alternatively halogen substituted benzoic acid derivative of general formula 2 (which is commercially available or described in the literature) can be converted to the corresponding azaquinazoline 7 in analogy to literature procedures. Typically derivative 2 is reacted with acetamidine, copper metal, a base such as for example potassium carbonate in an organic solvent such as for example DMF at elevated temperature. For example see WO2005/51410, US2008/107623 and references therein.

Step 3→7 (Scheme 1)

Azaquinalzoline Formation

Alternatively amino substituted benzoic acid derivative of general formula 3 (which is commercially available or described in the literature) can be converted to the corresponding azaquinazoline 7 in analogy to literature procedures. Typically derivative 3 is reacted with acetyl chloride or acetic anhydride, an ammonia source such as for example ammonia or ammonium acetate, a base such as for example triethylamine or pyridine with or without DMAP in an organic solvent such as for example DMF, toluene, 1,4-dioxane/water at elevated temperature. For example see Bioorganic and Medicinal Chemistry Letters, 2011, vol. 21, #4 p. 1270-1274; Bioorganic and Medicinal Chemistry Letters, 2010, vol. 20, #7 p. 2330-2334; WO2008/117079 or WO2006/74187 and references therein.

Step 4→7 (Scheme 1)

Azaquinazoline Formation

Alternatively benzoxazinone derivative of general formula 4 (which is commercially available or can be prepared in analogy to literature procedures) can be converted to the corresponding azaquinazoline 7 in analogy to literature procedures. Typically derivative 4 is reacted with ammonium acetate in a solvent at elevated temperature. For example see Bioorganic and Medicinal Chemistry Letters, 2011, vol. 21, #4 p. 1270-1274 or U.S. Pat. No. 6,350,750 and references therein.

Step 5→7 (Scheme 1)

Azaquinazoline Formation

Alternatively benzoic acid amide derivative of general formula 5 (which is commercially available or described in the literature) can be converted to the corresponding azaquinazoline 7 in analogy to literature procedures. Typically derivative 5 is reacted with a base such as for example sodium hydroxide in a solvent such as for example water at elevated temperature. For example see Bioorganic and Medicinal Chemistry Letters, 2008, vol. 18, #16 p. 4573-4577 and references therein.

Step 6→7 (Scheme 1)

Azaquinazoline Formation

Alternatively amino benzoic acid amide derivative of general formula 6 (which is commercially available or described in the literature) can be converted to the corresponding azaquinazoline 7 in analogy to literature procedures. Typically derivative 6 is reacted with acetic acid at elevated temperature. For example see Bioorganic and Medicinal Chemistry Letters, 2008, vol. 18, #3 p. 1037-1041 and references therein.

Step 7→8 (Scheme 1)

Conversion of Hydroxyl Group Into Leaving Group

In the next step (scheme 1) hydroxy azaquinazoline derivative 7 can be converted to the corresponding azaquinazoline 8 in analogy to literature procedures.

For W=chloro typically trichlorophosphate or thionylchloride, with or without N,N-dimethylaniline or N,N-diisopropylethylamine with or without an organic solvent such as for example toluene at elevated temperatures is used. For examples see Bioorganic and Medicinal Chemistry Letters, 2011, 1270; Journal of Medicinal Chemistry, 2009, 2341; ACS Medicinal Chemistry Letters, 2013, 846; Bioorganic and Medicinal Chemistry Letters, 2010, 2330; U.S. Pat. No. 6,350,750 or WO2015/54572 and references therein.

For W=bromo typically phosphorus oxytribromide, with or without N,N-dimethylaniline or N,N-diisopropylethylamine with or without an organic solvent such as for example toluene at elevated temperatures is used. For examples see US2012/53174; WO2012/30912 or WO2012/66122 and references therein.

For W=2,4,6-triisopropylsulfonate typically 2,4,6-triisopropylbenzenesulfonyl chloride, a base such as for example triethylamine and/or DMAP in an organic solvent such as for example dichloromethane is used. For examples see WO2010/99379 US2012/53176 and references therein.

For W=tosylate typically 4-methylbenzene-1-sulfonyl chloride, a base such as for example triethylamine or potassium carbonate and/or DMAP in an organic solvent such as for example dichloromethane or acetonitrile is used. For examples see Organic Letters, 2011, 4374 or Bioorganic and Medicinal Chemistry Letters, 2013, 2663 and references therein.

For W=trifluoromethanesulfonate typically N,N-bis(trifluoromethylsulfonyl)aniline or trifluoromethanesulfonic anhydride, a base such as for example triethylamine or 1,8-diazabicyclo[5.4.0]undec-7-ene and/or DMAP in an organic solvent such as for example dichloromethane is used. For examples see Journal of the American Chemical Society, 2015, 13433 or WO2014/100501and references therein.

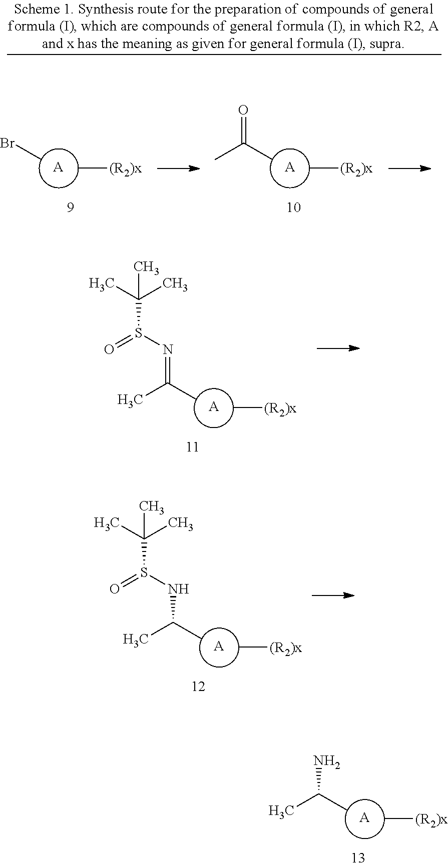

Step 9→10 (Scheme 1)

Acetyl Formation

In the first step (scheme 1) the bromo derivative 9 (which is commercially available or described in the literature) could be converted to the corresponding acetyl 10 in analogy to the numerous literature procedures. For example the reaction can be performed using different chemistries known to those skilled in the art, for example, Grignard chemistry using magnesium in an organic solvent as for example THF; or palladium catalyzed chemistry or Stille chemistry. For such transformations see the teachings of (Grignard: Fillon et al., Tetahedron 2003, 59, 8199; Leazer et al., Org. Synth. 2005, 82, 115; Palladium: WO2005/5382; Stille: WO2019/122129 and the references therein.

Step 10→11 (Scheme 1)

Sulfinimine Formation

In the first step (scheme 1) aldehyde derivative 10 (which is commercially available or described in the literature) could be converted to the corresponding sulfinimine 11 in analogy to the numerous literature procedures. For example the reaction could be performed at ambient temperature using Titanium(IV)ethoxide or Titanium(IV) isopropoxide in an organic solvent as for example THF. For a review about sulfinimine chemistry see for example Chem. Rev. 2010, 110, 3600-3740; Chem. Soc. Rev. 2009, 38, 1162-1186; Tetrahedron 2004, 60, 8003 or WO2019/122129 and the references therein.

Step 11→12 (Scheme 1)

Formation of Sulfinamide

In the next step (scheme 1) sulfinimine 11 can be converted to the corresponding sulfinamide 12 in analogy to the numerous literature procedures. For example the reaction can be performed using a reducing agent, for example, sodium borohydride or borane-THF, in a protic organic solvent as for example ethanol or methanol or tetrahydrofuran. Such transformations are known to those skilled in the art, see the teachings of Pan et al., Tetrahedron Asym., 2011, 22, 329; WO2019/122129; Li et al., Chem. Med. Chem., 2018, 13, 1363; Ghosh et al., Eur. J. Med. Chem., 2018, 160, 171. Alternatively, the reaction can be performed using a reducing agent, for example, diisopropylaluminium hydride, in an aprotic solvent, for example, toluene. Such transformations are known to those skilled in the art, see the teachings of WO2017/6282; Lee et al., Synlett., 2019, 30, 401.

Step 12→13 (Scheme 1)

Formation of Amine

In the next step (scheme 2) sulfinamide 12 can be converted to the corresponding amine 13 in analogy to the numerous literature procedures. For example the reaction can be performed using acetylchloride in a protic organic solvent as for example methanol. For a review about sulfinimine and sulfonamide chemistry see for example Chem. Rev. 2010, 110, 3600-3740; Chem. Soc. Rev. 2009, 38, 1162-1186; Tetrahedron 2004, 60, 8003 or WO2013030138 and the references therein.

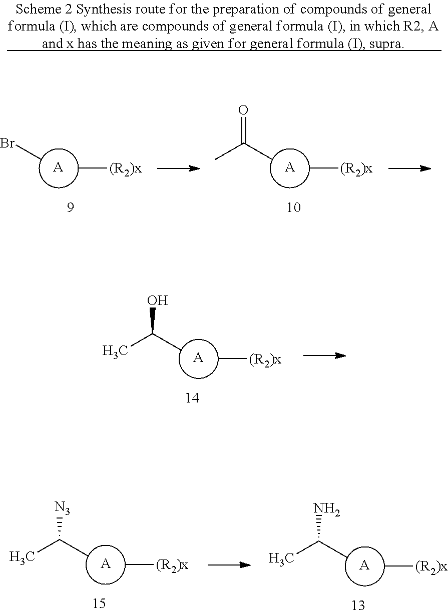

Step 10→14 (Scheme 2)

Formation of Alcohol

In the first step (scheme 2) ketone derivative 10 (which is commercially available or described in the literature) could be converted to the corresponding chiral alcohol 14 in analogy to the numerous literature procedures. For example the enanioselective reduction could be performed using catalytic hydrogenation, with hydrogen gas under pressure with a catalyst, for example a BINAP-derived catalyst, e.g. (R)- or (S)-RUCY-Xyl-BINAP (see WO2019/122129 page 140 or WO2013/185103 page 81).

Step 14→15 (Scheme 2)

Formation of Azide

In the next step (scheme 2) alcohol 14 can be converted to the corresponding azide 15 in analogy to the numerous literature procedures. For example the reaction can be performed using diphenylphosphonic azide and a base, for example, DBU, in an aprotic organic solvent as for example, toluene (see the teachings of WO2019/122129 page 144). For a review about azide chemistry see for example Chem. Rev. 1988, 88, 297.

Step 15→13 (Scheme 2)

Formation of Amine

In the next step (scheme 2) azide 15 can be converted to the corresponding amine 13 in analogy to the numerous literature procedures. For example the reaction can be performed using the Staudinger reduction conditions, with a phosphine, for example, triphenyl phosphine, in water with various different organic solvents, for example methanol, ethanol or THF. Alternatively, the azide reduction can be carried out using catalytic hydrogenation methods, using a metal catalyst, for example, palladium on charcoal, under a pressurized atmosphere of hydrogen (see WO2019/122129 page 144). For a review about azide chemistry see for example Chem. Rev. 1988, 88, 297.

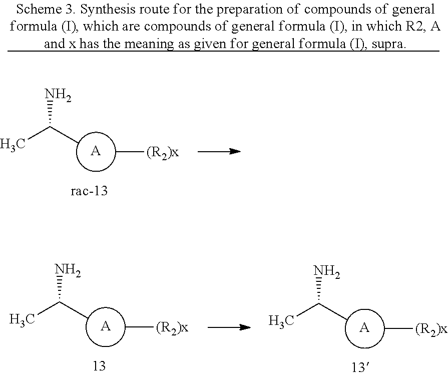

To those skilled in the art it is possible to carry out the chemical reactions described in Schemes 1 and 2, where the stereoisomers can be separated using various methods known to those skilled in the art, such as, for example, separation using chiral HPLC purification. The separation of these stereoisomers can be carried out on compounds of general formula 13.

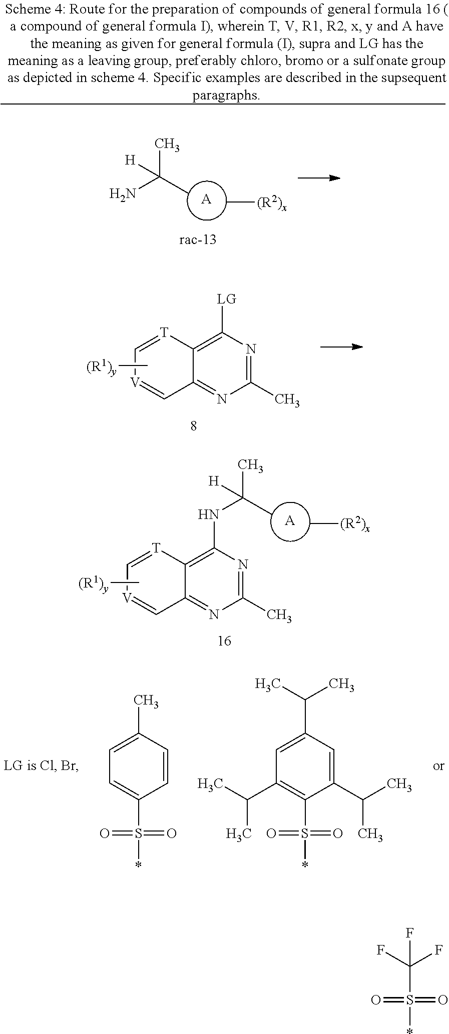

Step 12+8→17 (Scheme 4)

Amine Coupling

In the first step (scheme 4) amine derivative rac-13 and azaquinazoline derivative 8 are converted to amine 16 in analogy to literature procedures. Typically the reaction is performed in an organic solvent such as for example THF, DMF, acetonitrile dichloromethane or isopropyl alcohol with or without a base such as for example triethylamine, N-ethyl-N,N-diisopropylamine, potassium carbonate or potassium tert-butylate.

For LG=chloro see for example the literature references WO2008/86462; WO2008/86462 or European Journal of Medicinal Chemistry, 2015, 462 and references therein.

For LG=bromo see for example the literature references US2009/247519 or Journal of Organic Chemistry, 2009, 8460 and references therein.

For LG=tosylate see for example the literature references Synthetic Communications, 2012, 1715; Synthesis 2015, 2055 or Bioorganic and Medicinal Chemistry Letters, 2013, 2663 and references therein.

For LG=triflate see for example the literature references Bioorganic and Medicinal Chemistry Letters, 2013, 3325 and references therein.

For LG=2,4,6-triisopropylbenzenesulfonate see for example the literature reference WO2010/99379 and references therein.

In accordance with a further aspect, the present invention covers intermediate compounds which are useful in the preparation of compounds of the present invention of general formula (I), particularly in the methods described herein.

The present invention covers the intermediate compounds which are disclosed in the Example Section of this text, infra.

The present invention covers any sub-combination within any embodiment or aspect of the present invention of intermediate compounds.

In accordance with another aspect, the present invention covers methods of preparing compounds of the present invention, said methods comprising the step as described below and/or the Experimental Section.

The preparation of compounds of general formula I can be performed in a protic or aprotic solvent, preferably in dioxan, tetrahydrofuran, N,N-dimethylformamide, dimethylsulfoxid, methanol, ethanol or 2-propanol.

Preferred bases which can be used for the preparation of compounds of the general formula I are N,N-diisopropylethylamin or triethylamin.

Said compound of general formula I can then optionally be converted into solvates, salts and/or solvates of such salts using the corresponding (i) solvents and/or (ii) bases or acids.

The present invention covers methods of preparing compounds of the present invention of general formula (I), said methods comprising the steps as described in the Experimental Section herein.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

One of the most fundamental characteristics of cancer cells is their ability to sustain chronic proliferation whereas in normal tissues the entry into and progression through the cell division cycle is tightly controlled to ensure a homeostasis of cell number and maintenance of normal tissue function. Loss of proliferation control is emphasized as one of the six hallmarks of cancer [Hanahan D and Weinberg 15 RA, Cell 100, 57, 2000; Hanahan D and Weinberg R A, Cell 144, 646, 2011].

Compounds of general formula (I) of the present invention demonstrate a valuable pharmacological spectrum of action which could not have been predicted. Compounds of the present invention have surprisingly been found to effectively inhibit the Ras-Sos1 interaction and it is possible therefore that said compounds be used for the treatment or prophylaxis of diseases, preferably hyperproliferative disorders in humans and animals.

Compounds of the present invention can be utilized to inhibit, block, reduce, decrease, etc., cell proliferation and/or cell division, and/or produce apoptosis. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480 ; Peer et al., Lab. Invest., 1995, 72, 638], age-related macular degeneration (AMD) [Lopez et al., Invest. Opththalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal, intralumbal or intratumoral) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of a hyper-proliferative disorder, in particular cancer.

Particularly, the present invention covers a pharmaceutical combination, which comprises:
one or more first active ingredients, in particular compounds of general formula (I) as defined supra, and
one or more further active ingredients, in particular those used for treatment of hyper-proliferative disorder, in particular cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known anti-tumor agents (cancer therapeutics).

Examples of anti-tumor agents (cancer therapeutics) include:

131I-chTNT, abarelix, abiraterone, aclarubicin, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, darolutamide, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, larotrectinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumabpentetreotide, nilotinib, nilutamide, nimorazole, nimotuzumab, nimustine, nintedanib, nitracrine, nivolumab, obinutuzumab, octreotide, ofatumumab, olaparib, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rogaratinib, rolapitant, romidepsin, romiplostim, romurtide, roniciclib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Further examples of combination partners are ATR inhibitors (e.g. BAY 1895344), DHODH inhibitors (e.g. BAY 2402234), SHP2 inhibitors (e.g. SHP099, RMC-4550, TNO155) or H-, N- or K-Ras inhibitors, including inhibitors of mutants thereof, especially K-RAS-G12C inhibitors (e.g. ARS-853, ARS-1620, AMG-510, MRTX849, MRTX1257) or farnesyl transferase inhibitors.

In particular, the present invention covers a combination of a covalent inhibitor of KRAS-G12C and a SOS1 inhibitor. It has been shown that covalent KRAS-G12C inhibitors (e.g. ARS-853 or ARS-1620) specifically bind to KRAS-G12C in the GDP-bound state, but not in the GTP-bound state (Patricelli 2016 Cancer Discovery; Janes et al. 2018 Cell), thereby trapping KRAS-G12C in its inactive GDP-bound state. In addition, it has been shown that certain RAS mutants, which usually exist in the active, GTP-bound state, are undergoing a slow intrinsic GTP hydrolysis, in particular G12C and G12D mutants of KRAS (Hunter et al. 2015 Molecular Cancer Research). It can be postulated that even those mutant RAS proteins require the activation by nucleotide exchange factors like SOS1 for full activity and tumorigenesis. Treatment with a SOS1 inhibitor is expected to shift the intracellular equilibrium of KRAS mutants towards the inactive GDP-bound state, which in turn favours binding of inhibitors of KRAS which bind preferentially to the GDP-bound state of RAS, as is the case for covalent KRAS-G12C inhibitors like ARS-853 and ARS-1620. Synergistic anti-proliferative activity in vitro has been shown for the combination of BAY-293 with ARS-853 (Hillig 2019 PNAS).

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Experimental Section

The following table lists the abbreviations used in this paragraph, and in the examples section.

| | |
|---|---|
| BuLi | Butyllithium |
| DCE | Dichloroethane |
| DCM | Dichloromethane |
| DMF | Dimethylformamide |
| DMSO | Dimethyl sulfoxide |
| EA | Ethyl acetate |
| FA | Formic acid |
| HPLC, LC | high performance liquid chromatography |
| h | hour |
| LiHMDS | Lithium bis(trimethylsilyl)amide |
| KHMDS | Potassium bis(trimethylsilyl)amide |
| KOtBu | Potassium tert-butoxide |
| min | minute |
| LDA | Lithiumdiisopropylamid |
| MS | mass spectroscopy |
| NMR | nuclear magnetic resonance |
| NaHMDS | Sodium bis(trimethylsilyl)amide |
| PE | Petrol ether |
| Rac | Racemate |
| R_{f} | Retardiation factor |
| Rₜ | Retention time |
| RT | Room temperature |
| TFA | Trifluoroacetic acid |
| THF | Tetrahydrofuran |
| TLC | thin-layer chromatography |

Chemical names were generated using ACD/Name Batch Version 12.01 or Autonom 2000.

All reagents, for which the synthesis is not described in the experimental part, are either commercially available or synthesized as described in literature references.

Analytical Methods

LC-MS Method 1:
Column: Ascentis Express C18 2.7 μm, 30×2.1 mm
Fragment. potential: 50 V
Mass range: 80-800 m/z
Solvent: A=H₂O+0.1% vol HCOOH
   B=methanol+0.1% vol HCOOH
Gradient: 0-1 min 5% B, 1-4 min 5-100% B 4-5 min 100% B, 5-6 min 100-5% B, 6-6.5 min 5% B
Flow: 0.8 mL/min
Temperature: 30° C.
Injection: 1.0 μL
Detection: MM-ES+APCI+DAD (254 nm)
System time delay: 0.2 min

LC-MS Method 2: MS instrument type: Micromass Quatro Micro; HPLC instrument type: Agilent 1100 Series; UV DAD; column: Chromolith Flash RP-18E 25-2 mm; mobile phase A: 0.0375% TFA in water, mobile phase B: 0.01875% TFA in acetonitrile; gradient: 0.0 min 100% A→1.0 min 95% A→3.0 min 95% A→3.5 min 5% A→3.51 min 5% A→4.0 min 95% A; flow rate: 0.8 mL/min; column temp: 50° C.; UV detection: 220 nm & 254 nm.

LC-MS Method 3:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O+H₂O+0.1% vol. HCOOC (99%)
   B=acetonitrile
Gradient: 0-1.6 min 1-99% B, 1.6-2 min 99% B
Flow: 0.8 mL/min
Temperature: 60° C.
Injection: 2.0 μL
Detection: DAD scan range 210-400 nm+ELSD

LC-MS Method 4:
System: Shimadzu LC-MS: UFLC 20-AD and LCMS 2020 MS detector
Column: Shim-pack XR-ODS 2.2 μm, 3.0×50 mm
Solvent: A=H₂O+0.05% vol. HCOOC (99%)
   B=acetonitrile+0.05% vol. HCOOC (99%)

LC-MS Method 5:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O+0.2% vol. NH₃ (32%)
   B=acetonitrile
Gradient: 0-1.6 min 1-99% B, 1.6-2 min 99% B
Flow: 0.8 mL/min
Temperature: 60° C.
Injection: 2.0 μL
Detection: DAD scan range 210-400 nm+ELSD

LC-MS Method 6:
System: Instrument HPLC: Waters UPLC Acquity; Instrument MS: Waters ZQ
Column: Acquity UPLC BEH C18 1.7 μm, 50×2.1 mm
Solvent: A=H₂O+0.1% vol. HCOOC (99%)
   B=acetonitrile
Gradient: 0-1.6 min 1-99% B, 1.6-1.8 min 99% B, 1.81-2 min 1% B
Flow: 0.8 mL/min
Temperature: 60° C.
Detection: PDA scan range 210-400 nm

LC-MS Method 7:
System: Agilent 1290 UHPLC-MS Tof
Column: BEH C 18 (Waters) 1.7 μm, 50×2.1 mm
Solvent: A=H₂O+0.05% vol. HCOOC (99%)
   B=acetonitrile+0.05% vol. HCOOC (99%)
Gradient: 0-1.7 min 2-90% B, 1.7-2 min 90% B, 2-2.5 min 90-2% B
Flow: 1.2 mL/min
Temperature: 60° C.
Detection: DAD scan range 210-400 nm

LC-MS Method 8:
System: Waters Acquity UPLC-MS: Binary Solvent Manager, Sample Manager/Organizer, PDA, ELSD
Column: Acquity UPLC BEH C18 1.7 μm, 50×2.1 mm
Solvent: A=H₂O+0.1% vol. HCOOC (99%)
   B=acetonitrile
Gradient: 0-1.6 min 1-99% B, 1.6-2 min 99% B
Flow: 0.8 mL/min
Temperature: 60° C.
Injection: 2.0 μL
Detection: DAD scan range 210-400 nm+ELSD

LC-MS Method 9:
System: Waters Acquity UPLC-MS SingleQuad
Column: Kinetex C 18 (Phenomenex) 2.6 μm, 50×2.1 mm
Solvent: A=H₂O+0.05% vol. HCOOC (99%)
   B=acetonitrile+0.05% vol. HCOOC (99%)
Gradient: 0-0.2 min 2% B, 0.2-1.7 min 2-90% B, 1.7-1.9 min 90% B, 1.9-2 min 90-2% B, 2-2.5 min 2% B
Flow: 1.3 mL/min
Temperature: 60° C.
Detection: DAD scan range 210-400 nm

LC-MS method 10:

System: Waters Acquity UPLC-MS SingleQuad; Column: Acquity UPLC BEH C18 1.7 μm, 50×2.1 mm;
Solvent: A=H2O+0.2% vol. NH3 (32%), B=acetonitrile; Gradient: 0-1.6 min 1-99% B, 1.6-2 min 99% B; Flow: 0.8 mL/min; Temperature: 60° C.; Detection: DAD scan range 210-400 nm

Preparative HPLC

a) Autopurifier: Acidic Conditions
System: Waters Autopurification system: Pump 2545, Sample Manager 2767, CFO, DAD 2996, ELSD 2424, SQD
Column: XBrigde C18 5.0 μm 100×30 mm
Solvent: A=H₂O+0.1% vol. HCOOH (99%)
   B=acetonitrile
Gradient: 0-0.5 min 5% B 25 mL/min, 0.51-5.5 min 10-100% B 70 mL/min, 5.51-6.5 min 100% B 70 mL/min
Temperature: RT
Solution: max. 250 mg/max. 2.5 mL DMSO or DMF
Injection: 1×2.5 mL
Detection: DAD scan range 210-400 nm, MS ESI+, ESI−, scan range 160-1000 m/z

b) Autopurifier: Basic Conditions
System: Waters Autopurification system: Pump 2545, Sample Manager 2767, CFO, DAD 2996, ELSD 2424, SQD
Column: XBrigde C18 5.0 μm 100×30 mm
Solvent: A=H₂O+0.2% vol. NH₃ (32%)
   B=acetonitrile
Gradient: 0-0.5 min 5% B 25 mL/min, 0.51-5.5 min 10-100% B 70 mL/min, 5.51-6.5 min 100% B 70 mL/min
Temperature: RT
Solution: max. 250 mg/max. 2.5 mL DMSO or DMF
Injection: 1×2.5 mL
Detection: DAD scan range 210-400 nm, MS ESI+, ESI−, scan range 160-1000 m/z

Method X1:

Instrument: Labomatic HD5000, Labocord-5000; Gilson GX-241, Labcol Vario 4000; Column: Chiralpak IE 5 μm 250×20 mm; Eluent A: MTBE+0.1% vol. Diethylamine (99%); Eluent B: Ethanol; Isocratic: 90% A+10% B; Flow 30.0 mL/min; UV 254 nm.

Method X2:

Instrument: Labomatic HD5000, Labocord-5000; Gilson GX-241, Labcol Vario 4000; Column: Chiralpak IA 5 μm 250×30 mm; Eluent A: MTBE+0.1% vol. Diethylamine (99%); Eluent B: Ethanol; Isocratic: 85% A+15% B; Flow 40.0 mL/min; UV 254 nm.

Method X3:

Instrument: Labomatic HD5000, Labocord-5000; Gilson GX-241, Labcol Vario 4000, Column: Chiralpak IA 5.0 μm 250×30 mm; Eluent: 100% Acetonitrile; Flow 50.0 mL/min; UV 280 nm.

Method X4:

Instrument: Waters Autopurification system; Column: Waters XBrigde C18 5.0 μm 100×30 mm; Eluent A: H₂O+0.2% vol. NH₃ (32%), Eluent B: Acetonitrile; Gradient: 0.00-0.50 min 8% B (25->70 mL/min), 0.51-5.50 min 8-15% B (70 mL/min), DAD scan: 210-400 nm.

Method X5:

Instrument: Labomatic HD5000, Labocord-5000; Gilson GX-241, Labcol Vario 4000, Column: Chiralpak IF 5.0 μm 250×30 mm; Eluent A: Hexane+0.1% vol. Diethylamine (99%); Eluent B: Ethanol; Isocratic: 90% A+10% B; Flow 50.0 mL/min; UV 280 nm.

Method X6:

Instrument: Waters Autopurification system; Column: Waters XBrigde C18 5.0 μm 100×30 mm; Eluent A: H₂O+0.2% vol. NH₃ (32%), Eluent B: Acetonitrile; Gradient: 0.00-0.50 min 30% B (25->70 mL/min), 0.51-5.50 min 30-45% B (70mL/min), DAD scan: 210-400 nm.

Method X7:

Instrument: Labomatic HD5000, Labocord-5000; Gilson GX-241, Labcol Vario 4000,

Column: Chiralpak ID 5.0 μm 250×30 mm; Eluent A: Hexane+0.1% vol Diethylamin (99%); Eluent B: 2-Propanol; Isocratic: 85% A+15% B; Flow 50.0 mL/min; UV 254 nm.

Synthesis of Intermediates 13

Experimental Procedure [A] for the Synthesis of 13-a (See WO 2019/122129, Page 141, Line 2-Page 144, Line 1)

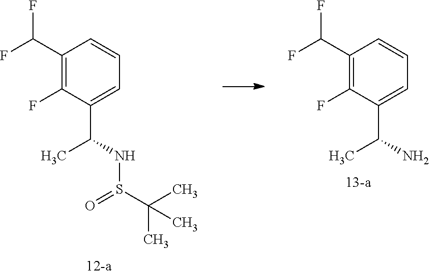 [SMILES: C[C@@H](NS(=O)C(C)(C)C)c1cccc(C(F)F)c1F ; C[C@@H](N)c1cccc(C(F)F)c1F]

A solution of 12-a (13.20 g, 45.00 mmol; 1.0 equiv.) in 1,4-dioxane (100 ml) is cooled to 0° C. and treated with 4 N HCl in 1,4-dioxane (50.00 ml, 200.00 mmol, 4.4 equiv.). The reaction mixture is stirred for 3 h. After complete conversion of the starting material, the reaction mixture is concentrated under reduced pressure, the precipitate filtered and washed with diethyl ether to obtain the desired product 13-a as HCl salt.

The crude product 13 is purified by chromatography if necessary and isolated as HCl salt.

Experimental Procedure [B] for the Synthesis of B-5k (See WO 2019/122129, Page 144, Line 2-Page 146, Line 1)

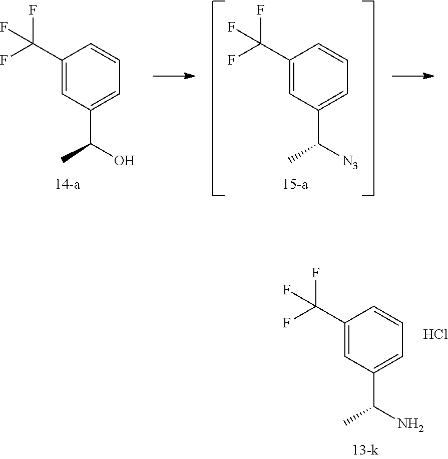

Alcohol 14 (2.00 g, 9.61 mmol, 1.0 equiv.) is dissolved in anhydrous toluene (20 mL).

Diazabicycloundecene (1.73 mL, 11.5 mmol, 1.2 equiv.) and diphenylphosphonic azide (2.28 mL, 10.6 mmol, 1.1 equiv.) are added subsequently. The reaction mixture is stirred at 40° C. for 18 h until complete conversion of 14 is achieved. The reaction mixture is cooled to room temperature and the organic layer is washed with aqueous Na₂CO₃ solution (2×10 mL). Azide B-7a thus obtained is not isolated but directly converted in the next step.

Pd/C (200 mg, 10% w/w, 10% Pd) is added to the organic layer. The reaction mixture is charged with a H₂ atmosphere (10 bar) and is stirred for 24 h until complete conversion of 15 is achieved. The reaction is filtered and the volatiles are removed in vacuo. The residue is dissolved in methyl tert-butyl ether (30 mL) and treated with HCl in dioxane (4.8 mL, 4 M). The white precipitate is filter, washed with methyl tert-butyl ether (20 mL) and further dried in vacuo to furnish the desired product 13. The crude product is purified by chromatography if necessary.

Table 1: Intermediates 13 (benzyl amines) available in analogous manner starting from different sulfonamides 12 (experimental procedure [A], table 1, column 2) or alcohols 14 via azides 15 (experimental procedure [B], table 1, column 3)

TABLE 1

| | A | B |
|---|---|---|
| 13-a: (1R)-1-[3-(difluoromethyl)-2-fluoro-phenyl]ethanamine | x | |
| 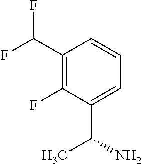 | | |
| 13-b: (1R)-1-[3-(difluoromethyl)-2-methyl-phenyl]ethanamine | x | |
| 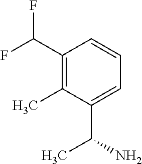 | | |
| 13-c | x | |
| 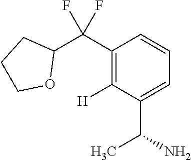 [SMILES: C[C@@H](N)c1cccc(C(F)(F)C2CCCO2)c1] | | |
| 13-d: (1R)-1-[3-[difluoro-[(2S)-tetrahydrofuran-2-yl]methyl]-2-fluoro-phenyl]ethanamine | x | |
| 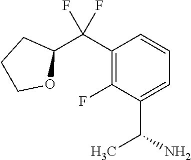 [SMILES: C[C@@H](N)c1cccc(C(F)(F)[C@@H]2CCCO2)c1F] | | |
| 13-e: (1R)-1-[3-[difluoro-[(2R)-tetrahydrofuran-2-yl]methyl]-2-fluoro-phenyl]ethanamine | x | |
| 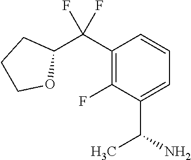 [SMILES: C[C@@H](N)c1cccc(C(F)(F)[C@H]2CCCO2)c1F] | | |
| 13-f: 1-[3-[(1R)-1-aminoethyl]-2-fluoro-phenyl]-1,1-difluoro-2-methyl-propan-2-ol | x | |
| 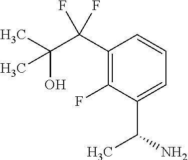 [SMILES: C[C@@H](N)c1cccc(C(F)(F)C(C)(C)O)c1F] | | |
| 13-g: (1R)-1-(1,1-difluoroindan-4-yl)ethanamine | x | |
| 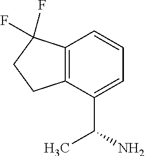 | | |
| 13-h: (1R)-1-[3-(1,1-difluoroethyl)phenyl]ethanamine | x | |
| 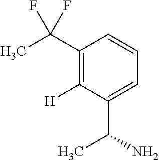 | | |
| 13-i: (1R)-1-[3-(1,1-difluoroethyl)-2-fluoro-phenyl]ethanamine | x | x |
| 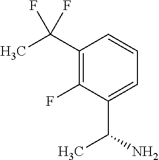 | | |
| 13-j: 2-[3-[(1R)-1-aminoethyl]-2-fluoro-phenyl]-2,2-difluoro-ethanol | x | |
| 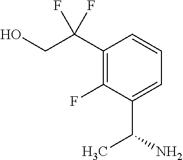 | | |
| 13-k: (1R)-1-[3-(trifluoromethyl)phenyl]ethanamine | x | x |
| 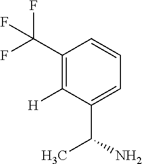 | | |
| 13-l: (1R)-1-[2-fluoro-3-(trifluoromethyl)phenyl]ethanamine | x | x |
| 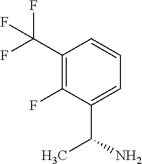 | | |
| 13-m: (1R)-1-[2-methyl-3-(trifluoromethyl)phenyl]ethanamine | x | x |
| 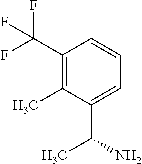 | | |
| 13-n: (1R)-1-[3-(2,2,2-trifluoroethyl)phenyl]ethanamine | x | |
| 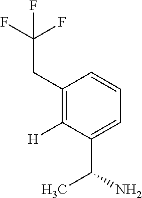 | | |
| 13-o: (1R)-1-(3,3-difluoro-2H-benzofuran-7-yl)ethanamine | x | |
| 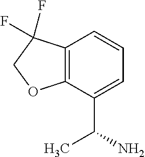 | | |
| 13-p: (1R)-1-(3-fluorobenzofuran-7-yl)ethanamine | x | |
| 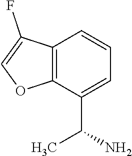 | | |

The synthesis of the different necessary sulfonamides B-4 is described in WO 2019/122129 at page 136 line 2 to page 140 line 9.

The synthesis of the different necessary alcohols B-6 is described in WO 2019/122129 at page 140 line 10 to page 141 line 1 (incl. table 14).

Intermediate 1

### 1-bromo-3-(difluoromethyl)-2-fluorobenzene

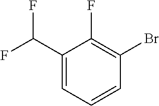 [SMILES: Fc1c(Br)cccc1C(F)F]

To a solution of 3-bromo-2-fluorobenzaldehyde (4.07 g, 20.1 mmol) in DCM (35 ml) at 0° C. was added slowly dropwise a solution of N-ethyl-N-(trifluoro-lambda⁴-sulfanyl)ethanamine (4.0 ml, 30 mmol) in DCM (10 ml). The reaction was allowed to warm and stirred at RT overnight. The reaction mixture was added to ice-water and extracted with DCM. The organics were combined, washed with sat. NaCl(aq), filtered through an hydrophobic filter and concentrated under reduced pressure. The residue was purified by silica chromatography (Hexane:EtOAc) and gave the titled compound (3.57 g, 75%).

¹H-NMR (400 MHz, DMSO-d6) δ [ppm]: 2.518 (0.97), 2.522 (0.62), 7.113 (7.95), 7.248 (16.00), 7.303 (4.71), 7.323 (9.95), 7.343 (5.61), 7.383 (7.82), 7.642 (3.92), 7.659 (6.89), 7.678 (3.45), 7.911 (3.70), 7.928 (6.59), 7.948 (3.45).

Intermediate 2

### 1-[3-(difluoromethyl)-2-fluorophenyl]ethan-1-one

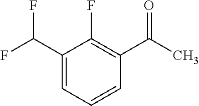

To a solution of 1-bromo-3-(difluoromethyl)-2-fluorobenzene (3.07 g, 13.6 mmol) in anhydrous THF (10 ml) at −10° C. was added isopropylmagnesium chloride (2M in THF, 7.5 ml, 15 mmol). The reaction was stirred at −10° C. for 1 h and then to added to acetic anhydride (3.9 ml, 41 mmol) cooled to −15° C.

The reaction was was to warm to 0° C. and stirred for 15 min. The reaction was quenched by the addition of water and stirred at 60° C. for 15 min. The reaction mixture was extracted with DCM. The organics were combined, washed with sat. NaHCO3(aq), sat. NaCl(aq), filtered through an hydrophobic filter and concentrated under reduced pressure. The crude product (787 mg, 28%) was used directly without any further purification.

Intermediate 3

### (R)-N-{1-[3-(difluoromethyl)-2-fluorophenyl]ethylidene}-2-methylpropane-2-sulfinamide

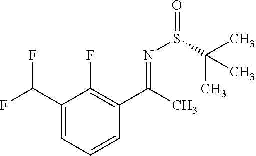 [SMILES: C/C(=N\[S@](=O)C(C)(C)C)c1cccc(C(F)F)c1F]

To a solution of 1-[3-(difluoromethyl)-2-fluorophenyl]ethan-1-one (787 mg, 4.18 mmol) and (R)-2-methyl-2-propane-2-sulfinamide (760 mg, 6.27 mmol) was added Ti(OEt)4 (2.86 g, 12.5 mmol) and heated at 80° C. overnight. The reaction was added to a mixture of EtOAc and ice-water and extracted with EtOAc. The organics were combined, filtered through an hydrophobic filter and concentrated under reduced pressure. The residue (1.31 g, 97%) was used directly in the next step.

Intermediate 4

### (R)-N-{(1R)-1-[3-(difluoromethyl)-2-fluorophenyl]ethyl}-2-methylpropane-2-sulfinamide

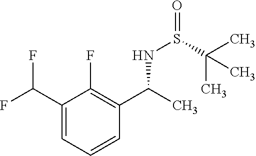 [SMILES: C[C@@H](N[S@](=O)C(C)(C)C)c1cccc(C(F)F)c1F]

To a solution of (R)-N-{1-[3-(difluoromethyl)-2-fluorophenyl]ethylidene}-2-methylpropane-2-sulfinamide (1218 mg, 4.18 mmol) in THF (12 ml) was cooled to 0° C. and NaBH4 (158 mg, 4.18 mmol) was added. The reaction was stirred at RT for 2h. The reaction was added to a mixture of EtOAc and ice-water, then extracted with EtOAc. The organics were combined, filtered through an hydrophobic filter and concentrated under reduced pressure. The titled compound (802 mg, 62%) was obtained after silica chromatography (EtOAc:Hexane) along with its diastereoisomer (166 mg, 13%).

¹H-NMR (400 MHz, DMSO-d6) δ [ppm]: 1.099 (16.00), 1.154 (0.44), 1.172 (0.85), 1.190 (0.42), 1.401 (2.11), 1.418 (2.10), 1.987 (1.59), 5.870 (0.54), 5.889 (0.52), 7.074 (0.41), 7.209 (0.86), 7.345 (1.03).

Intermediate 5

### (1R)-1-[3-(difluoromethyl)-2-fluorophenyl]ethan-1-amine, salt with hydrogen chloride

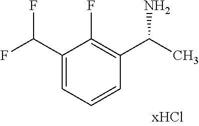

To an ice-cooled solution of (R)-N-{(1R)-1-[3-(difluoromethyl)-2-fluorophenyl]ethyl}-2-methylpropane-2-sulfinamide (1.00 g, 3.41 mmol) in dioxane (7.5 ml) was added HCl (4M in dioxane, 3.75 ml). The reaction was allowed to warm to RT and stirred for 3 h. The reaction mixture was concentrated under reduced pressure to approximately a volume of about 2 ml. The solid was collected by filtered and was washed with MTBE and the titled compound (618 mg, 76%) was obtained.

¹H-NMR (400 MHz, DMSO-d6) δ [ppm]: 1.102 (7.29), 1.532 (7.14), 1.549 (7.00), 2.518 (0.81), 2.523 (0.57), 3.072 (2.53), 3.565 (5.88), 4.636 (0.46), 4.653 (1.59), 4.670 (1.66), 4.681 (0.63), 4.686 (0.58), 5.760 (16.00), 7.119 (2.25), 7.254 (4.53), 7.388 (2.02), 7.429 (1.08), 7.449 (2.38), 7.468 (1.37), 7.651 (1.03), 7.669 (1.76), 7.687 (0.86), 7.888 (0.87), 7.906 (1.16), 7.925 (0.54), 8.584 (0.43), 8.709 (1.89).

Intermediate 6

### 6-Fluoro-2-methylpyrido[3,4-d]pyrimidin-4-ol

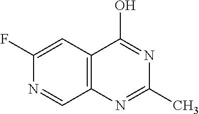

A round-bottom flask was charged with 5.00 g (32.0 mmol, commercially available) 5-Amino-2-fluoro-4-pyridinecarboxylic acid, 7.57 g (80 mmol, commercially available) acetamidine hydrochloride, and 6.56 g (80 mmol) anhydrous sodium acetate. The mixture was suspended in 50.0 ml of 2-methoxyethanol, and then the mixture was stirred at 130° C. for 16 h. The course of the reaction was monitored by LC/MS. Complete conversion was observed. The resulting mixture was poured into cold water and stirred for 30 min. The precipitate was filtered off and dried in vacuo. 5.95 g (98% d. Th.) of the title compound was obtained in form of a beige-coloured solid.

1H-NMR (400 MHz, DMSO-d6): [symbol] [ppm]=13.14−11.96 (br s, 1H), 8.66 (s, 1H), 7.59 (d, 1H), 2.37 (s, 3H).

Intermediate 7

### 6-ethoxy-2-methylpyrido[3,4-d]pyrimidin-4-ol

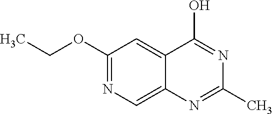

A round-bottom flask was charged with ethanol (110 ml) and cooled with an ice bath. To the ethanol was carefully added sodium (3.73 g, 163 mmol) and stirred for 5 min. 6-fluoro-2-methylpyrido[3,4-d]pyrimidin-4-ol (5.85 g, 32.7 mmol) was added and the mixture was stirred at 110° C. for 16 h. The course of the reaction was monitored by LC/MS, nearly complete conversion was detected. The solution was cooled to room temperature and concentrated in vacuo. Under cooling in an ice-bath the residue was diluted with 500 ml of water, then acidified with 2M hydrochloric acid (200 mL) to pH=1 and extracted with dichloromethane (2×200 ml) and a mixture of dichlormethane/isopropanol (4:1, 5×200 ml). The combined organic layers were dried over sodium sulfate and then concentrated in vacuo. The title compound (4.83 g, 77%) was obtained in form of a beige/brown-coloured solid.

1H-NMR (400 MHz, DMSO): [symbol] [ppm]=8.62 (s, 1H), 7.17 (s, 1H), 4.34 (q, 2H), 1.34 (t, 3H).

Intermediate 8

### 6-methoxy-2-methylpyrido[3,4-d]pyrimidin-4-ol

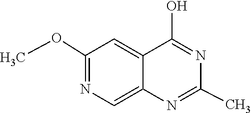

A mixture of 5-amino-2-methoxypyridine-4-carboxylic acid (2.50 g, 14.9 mmol), ethanimidamide hydrochloride (2.81 g, 29.7 mmol) and anhydrous sodium acetate (2.44 g, 29.7 mmol) in 2-methoxyethanol (40 ml) was heated under reflux conditions for 6 h. The solution was cooled to room temperature and water (50 ml) was added. The precipitate was collected by filtration, washed with water and dried in vacuo to give the titled compound (2.31 g).

1H-NMR (400 MHz, DMSO): [symbol] [ppm]=2.27 (br s, 1H), 8.60 (d, 1H), 7.19 (d, 1H), 3.79-3.98 (s, 3H), 2.32 (s, 3H).

Intermediate 9

### N-[(3R)-1-(4-hydroxy-2-methylpyrido[3,4-d]pyrimidin-6-yl)pyrrolidin-3-yl]acetamide

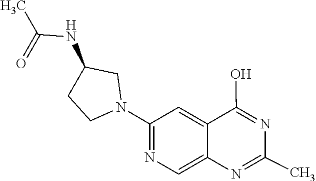 [SMILES: CC(=O)N[C@@H]1CCN(c2cc3c(O)nc(C)nc3cn2)C1]

A mixture of 6-fluoro-2-methylpyrido[3,4-d]pyrimidin-4-ol (10.0 g, 55.8 mmol) and N-[(3R)-pyrrolidin-3-yl]acetamide (12.5 g, 97.7 mmol in DMSO (40 ml) was added triethylamine (23 ml, 170 mmol) and heated at 90° C. for 16 h. The reaction mixture was concentrated under reduced pressure and the residue purified by silica chromatography (DCM:EtOH) to give the titled compound (13.56g, 80%).

¹H-NMR (400 MHz, DMSO-d6) δ [ppm]: 1.035 (2.29), 1.052 (4.89), 1.070 (2.52), 1.807 (16.00), 1.898 (0.76), 1.911 (0.63), 1.922 (0.47), 1.929 (0.47), 2.159 (0.51), 2.174 (0.60), 2.190 (0.55), 2.205 (0.43), 2.258 (0.80), 2.284 (13.66), 2.522 (1.32), 2.539 (4.91), 2.669 (0.43), 3.288 (0.67), 3.297 (0.72), 3.314 (0.92), 3.417 (0.41), 3.421 (1.02), 3.434 (1.07), 3.439 (1.07), 3.452 (1.13), 3.457 (0.54), 3.469 (0.53), 3.484 (0.62), 3.497 (0.71), 3.504 (0.71), 3.513 (0.72), 3.531 (1.07), 3.549 (0.56), 3.556 (0.49), 3.635 (0.82), 3.650 (0.97), 3.662 (0.83), 3.677 (0.77), 4.345 (1.31), 4.358 (1.96), 4.370 (0.96), 5.758 (0.45), 6.737 (3.67), 8.162 (1.02), 8.179 (1.01), 8.571 (4.12), 12.085 (0.80).

Intermediate 10

### N-[(3S)-1-(4-hydroxy-2-methylpyrido[3,4-d]pyrimidin-6-yl)pyrrolidin-3-yl]acetamide

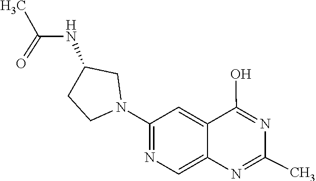 [SMILES: CC(=O)N[C@H]1CCN(c2cc3c(O)nc(C)nc3cn2)C1]

Analogously to Intermediate 9 using N-[(3S)-pyrrolidin-3-yl]acetamide (2.15 g, 16.7 mmol) gave the titled compound (1.06 g, 63%) after silica chromatography (DCM:EtOH).

¹H-NMR (400 MHz, DMSO-d6) δ [ppm]: 1.035 (2.38), 1.052 (5.02), 1.069 (2.64), 1.807 (16.00), 1.898 (0.70), 1.912 (0.55), 2.159 (0.45), 2.174 (0.53), 2.190 (0.47), 2.283 (13.64), 2.518 (0.44), 3.287 (0.65), 3.297 (0.72), 3.314 (1.02), 3.337 (4.95), 3.428 (0.67), 3.445 (0.66), 3.482 (0.55), 3.495 (0.62), 3.502 (0.60), 3.513 (0.66), 3.547 (0.49), 3.555 (0.44), 3.634 (0.76), 3.649 (0.89), 3.660 (0.77), 3.676 (0.70), 4.347 (0.64), 4.361 (0.64), 5.758 (1.76), 6.732 (3.35), 6.734 (3.31), 8.161 (0.91), 8.177 (0.89), 8.567 (3.83), 8.568 (3.81).

Intermediate 11

### 2-methyl-6-(4-methylpiperazin-1-yl)pyrido[3,4-d]pyrimidin-4-ol

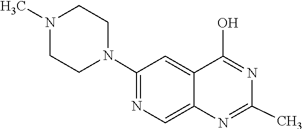

Analogously to Intermediate 9 using 1-methylpiperazine (2.24 g, 22.3 mmol) gave the titled compound (1.69 g, 55%) after silica chromatography (DCM:EtOH).

¹H-NMR (400 MHz, DMSO-d6) δ [ppm]: 1.052 (0.49), 2.178 (0.53), 2.219 (13.17), 2.296 (16.00), 2.404 (2.87), 2.417 (3.99), 2.430 (3.10), 2.518 (1.22), 2.523 (0.83), 3.509 (2.77), 3.522 (3.47), 3.535 (2.74), 7.110 (3.66), 8.592 (4.09), 12.145 (0.89).

Intermediate 12

### 6-[(3R)-3-(dimethylamino)pyrrolidin-1-yl]-2-methylpyrido[3,4-d]pyrimidin-4-ol

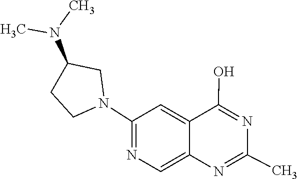

Analogously to Intermediate 9 using (3R)-N,N-dimethylpyrrolidin-3-amine (2.55 g, 22.3 mmol) gave the titled compound (2.17 g, 68%) after silica chromatography (DCM:EtOH).

¹H-NMR (400 MHz, DMSO-d6) δ [ppm]: 1.824 (0.41), 2.206 (16.00), 2.279 (9.34), 2.288 (0.61), 3.131 (0.58), 3.152 (0.65), 3.155 (0.72), 3.176 (0.56), 3.364 (0.79), 3.381 (0.62), 3.390 (0.41), 3.619 (0.53), 3.694 (0.40), 3.712 (0.48), 3.719 (0.46), 6.751 (2.42), 6.753 (2.36), 8.555 (2.60), 8.557 (2.56).

Intermediate 13

### 2-(4-hydroxy-2-methylpyrido[3,4-d]pyrimidin-6-yl)-2,6-diazaspiro[3.4]octan-7-one

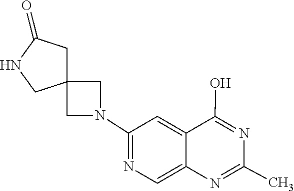 [SMILES: Cc1nc(O)c2cc(N3CC4(CNC(=O)C4)C3)ncc2n1]

Analogously to Intermediate 9 using 2,6-diazaspiro[3.4]octan-7-one oxalate salt (4.83 g, 22.3 mmol) gave the titled compound (1 g, 30%) after silica chromatography (DCM:EtOH).

¹H-NMR (400 MHz, DMSO-d6) δ [ppm]: 1.035 (1.25), 1.052 (2.83), 1.069 (1.11), 2.290 (16.00), 2.327 (0.45), 2.518 (2.47), 2.523 (1.44), 2.539 (8.49), 2.669 (0.48), 3.165 (6.18), 3.336 (0.51), 3.411 (0.68), 3.428 (1.27), 3.445 (1.25), 3.463 (0.57), 3.982 (15.13), 6.737 (4.83), 6.739 (4.60), 7.675 (1.53), 8.562 (4.66), 8.565 (4.55), 12.151 (0.73).

Intermediate 14

### 1-[4-(4-hydroxy-2-methylpyrido[3,4-d]pyrimidin-6-yl)piperazin-1-yl]ethan-1-one

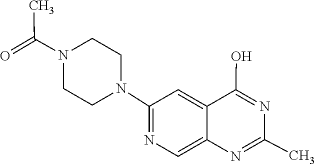 [SMILES: CC(=O)N1CCN(c2cc3c(O)nc(C)nc3cn2)CC1]

Analogously to Intermediate 9 using 1-(piperazin-1-yl)ethan-1-one (2.38 g, 18.6 mmol) gave the titled compound (511 g, 16%) after silica chromatography (DCM:EtOH).

¹H-NMR (400 MHz, DMSO-d6) δ [ppm]: 1.052 (0.58), 2.050 (16.00), 2.301 (14.44), 2.518 (1.23), 2.523 (0.89), 2.540 (2.37), 3.523 (1.33), 3.532 (1.43), 3.538 (1.95), 3.563 (3.09), 3.578 (3.59), 3.595 (2.31), 7.146 (3.41), 7.148 (3.39), 8.615 (3.86), 12.174 (0.84).

Intermediate 15

### 7-chloro-2-methylpyrido[4,3-d]pyrimidin-4-ol

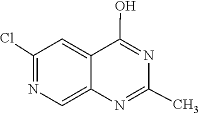

To a solution of 5-amino-2-chloropyridine-4-carboxylic acid (100 g, 579 mmol) and ethanimidamide hydrochloride (164 g, 1.74 mol) in 2-methoxyethanol (1.2 L) was added sodium acetate (143 g, 1.74 mol) at room temperature. The reaction mixture was stirred at 130° C. for 48 hours. The reaction mixtrue was concentrated to remove about 400 ml 2-methoxyethanol under reduced pressure. The residue was poured into water, brown solid was precipitated. The precipitates were filtered, dried under reduced pressure by oil pump to give 7-chloro-2-methylpyrido[4,3-d]pyrimidin-4-ol as a brown solid (77 g, 67%)

¹H-NMR (400 MHz, DMSO-d6) δ [ppm]: 2.384 (16.00), 2.518 (0.89), 2.523 (0.59), 7.928 (4.21), 7.930 (4.17), 8.817 (3.76), 8.819 (3.55).

Intermediate 16

### 1-(4-hydroxy-2-methylpyrido[3,4-d]pyrimidin-6-yl)piperidine-4-carbonitrile

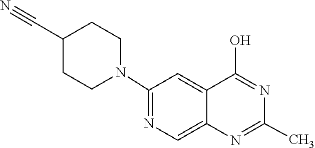 [SMILES: Cc1nc(O)c2cc(N3CCC(C#N)CC3)ncc2n1]

Analogously to Intermediate 9 using 1 piperidine-4-carbonitrile (2.46 g, 22.3 mmol) gave the titled compound (1.51 g, 48%) gave the title compound after silica chromatography (DCM:EtOH).

¹H-NMR (400 MHz, DMSO-d6) δ [ppm]: 1.052 (0.81), 1.070 (0.41), 1.722 (0.42), 1.734 (0.75), 1.744 (1.07), 1.755 (0.93), 1.767 (1.26), 1.776 (1.05), 1.789 (0.60), 1.798 (0.52), 1.919 (0.47), 1.928 (0.95), 1.936 (1.02), 1.944 (0.99), 1.952 (0.87), 1.961 (0.74), 1.968 (0.75), 1.976 (0.71), 2.296 (16.00), 2.518 (0.97), 2.523 (0.66), 3.114 (0.51), 3.124 (0.74), 3.134 (0.98), 3.145 (0.73), 3.156 (0.49), 3.393 (0.81), 3.401 (0.94), 3.414 (0.91), 3.426 (1.28), 3.435 (1.20), 3.448 (1.08), 3.456 (0.95), 3.820 (0.81), 3.830 (1.04), 3.836 (0.99), 3.846 (0.90), 3.854 (0.81), 3.863 (0.86), 3.870 (0.91), 3.879 (0.70), 5.758 (0.61), 7.157 (3.88), 8.599 (4.25), 12.156 (0.92).

Intermediate 17

### 6-[(2S)-2,4-dimethylpiperazin-1-yl]-2-methylpyrido[3,4-d]pyrimidin-4-ol

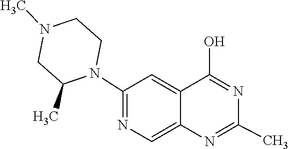

Analogously to Intermediate 9 using (3S)-1,3-dimethylpiperazine (153 mg, 1.34 mmol) gave the titled compound (30 mg, 16%) gave the title compound after preparative HPLC purification (basic method).

¹H-NMR (400 MHz, DMSO-d6) δ [ppm]: 1.120 (6.27), 1.137 (6.28), 1.751 (0.45), 1.921 (0.49), 1.941 (0.71), 1.950 (0.75), 1.971 (0.53), 1.979 (0.42), 2.114 (0.77), 2.125 (0.91), 2.142 (0.91), 2.152 (0.83), 2.201 (11.69), 2.291 (16.00), 2.304 (0.42), 2.518 (3.07), 2.523 (2.26), 2.702 (1.07), 2.729 (0.99), 2.843 (0.68), 2.871 (0.64), 3.017 (0.43), 3.025 (0.49), 3.048 (0.91), 3.056 (0.83), 3.079 (0.52), 3.957 (0.61), 3.989 (0.57), 4.535 (0.56), 7.036 (3.56), 8.595 (3.91).

Intermediate 18

### 6-[2-(hydroxymethyl)-4-methylpiperazin-1-yl]-2-methylpyrido[3,4-d]pyrimidin-4-ol (mixture of stereoisomers)

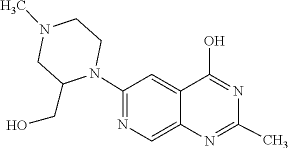

Analogously to Intermediate 9 using [4-methylpiperazin-2-yl]methanol (218 mg, 1.67 mmol) gave the titled compound (40 mg, 17%) gave the title compound after preparative HPLC purification (basic method).

¹H-NMR (400 MHz, DMSO-d6) δ [ppm]: 1.751 (0.99), 1.909 (0.46), 1.930 (0.67), 1.938 (0.70), 1.960 (1.09), 1.969 (1.06), 1.988 (0.85), 1.998 (0.74), 2.066 (0.78), 2.118 (0.78), 2.200 (10.96), 2.287 (16.00), 2.302 (0.49), 2.306 (0.46), 2.518 (3.49), 2.523 (2.57), 2.815 (0.67), 2.843 (0.60), 2.997 (0.46), 3.019 (0.81), 3.027 (0.74), 3.052 (1.30), 3.081 (0.95), 3.727 (0.49), 3.739 (0.49), 4.072 (0.56), 4.103 (0.53), 4.285 (0.53), 4.627 (0.42), 4.770 (0.60), 7.072 (3.49), 8.563 (4.23).

Intermediate 19

### 2-methyl-6-[2-(trifluoromethyl)-5,6-dihydroimidazo[1,2-a]pyrazin-7(8H)-yl]pyrido[3,4-d]pyrimidin-4-ol

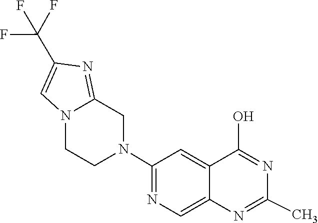 [SMILES: Cc1nc(O)c2cc(N3CCn4cc(C(F)(F)F)nc4C3)ncc2n1]

Analogously to Intermediate 9 using 2-(trifluoromethyl)-5,6,7,8-tetrahydroimidazo[1,2-a]pyrazine (128 mg, 670 μmol) gave the title compound (40 mg, 34%) after preparative HPLC purification (basic method).

¹H-NMR (400 MHz, DMSO-d6) δ [ppm]: 2.315 (16.00), 2.327 (1.22), 2.332 (0.79), 2.518 (4.14), 2.523 (2.82), 2.665 (0.66), 2.669 (0.91), 2.673 (0.64), 4.152 (3.17), 4.162 (3.14), 4.821 (7.48), 7.335 (3.95), 7.805 (2.85), 7.808 (2.91), 8.673 (4.41).

Intermediate 20

### 2-methyl-6-[2-(trifluoromethyl)-5,6-dihydro[1,2,4]triazolo[1,5-a]pyrazin-7(8H)-yl]pyrido[3,4-d]pyrimidin-4-ol

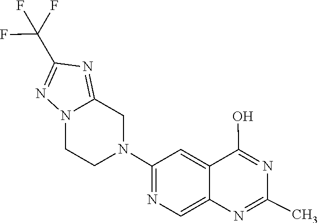 [SMILES: Cc1nc(O)c2cc(N3CCn4nc(C(F)(F)F)nc4C3)ncc2n1]

Analogously to Intermediate 9 using 2-(trifluoromethyl)-5,6,7,8-tetrahydro[1,2,4]triazolo[1,5-a]pyrazine (215 mg, 1.12 mmol) gave the title compound (25 mg, 13%) after preparative HPLC purification (basic method).

¹H-NMR (400 MHz, DMSO-d6) δ [ppm]: 1.056 (0.83), 1.071 (0.88), 1.752 (0.45), 2.320 (16.00), 2.430 (0.80), 2.518 (7.83), 2.523 (5.50), 2.540 (1.66), 2.665 (0.77), 2.669 (1.03), 2.673 (0.77), 4.260 (1.23), 4.272 (2.59), 4.286 (2.00), 4.373 (1.76), 4.386 (2.40), 4.400 (1.13), 4.997 (6.61), 7.422 (3.99), 7.424 (3.99), 8.088 (0.45), 8.681 (4.22), 8.683 (4.22).

### EXAMPLE 1

### N-{(1R)-1-[3-(difluoromethyl)-2-fluorophenyl]ethyl}-6-ethoxy-2-methylpyrido[3,4-d]pyrimidin-4-amine

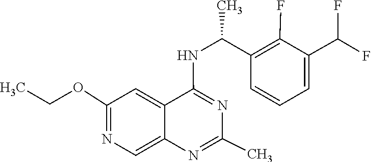 [SMILES: CCOc1cc2c(N[C@H](C)c3cccc(C(F)F)c3F)nc(C)nc2cn1]

To a solution of 6-ethoxy-2-methylpyrido[3,4-d]pyrimidin-4-ol (75.0 mg, 365 μmol) and 2,4,6-tri(propan-2-yl)benzene-1-sulfonyl chloride (188 mg, 621 μmol) was added triethylamine (180 μl, 1.3 mmol) followed by DMAP (6.70 mg, 54.8 μmol) and stirred at RT for 1 h. To the reaction was added (1R)-1-[3-(difluoromethyl)-2-fluorophenyl]ethan-1-amine hydrochloride (99.0 mg, 439 μmol) and stirred at RT overnight. The reaction was diluted with water and DCM and extracted with DCM. The organics were combined, washed with sat. NaCl(aq), filtered through an hydrophobic filter and concentrated under reduced pressure. The residue was purified by preparative HPLC (basic method) and gave the titled compound (14 mg, 10%).

¹H-NMR (400 MHz, DMSO-d6) δ [ppm]: 1.349 (2.26), 1.366 (5.02), 1.384 (2.32), 1.586 (2.95), 1.604 (2.94), 2.322 (0.41), 2.326 (0.58), 2.331 (0.60), 2.342 (8.54), 2.518 (1.99), 2.522 (1.25), 2.669 (0.49), 4.337 (0.68), 4.355 (2.20), 4.372 (2.13), 4.389 (0.63), 5.742 (0.52), 5.758 (16.00), 5.777 (0.45), 7.098 (0.64), 7.234 (1.31), 7.268 (0.48), 7.287 (1.04), 7.306 (0.60), 7.370 (0.58), 7.502 (0.64), 7.667 (0.63), 7.745 (2.20), 8.567 (0.72), 8.585 (0.69), 8.698 (2.49).

### EXAMPLE 2

### N-{(1R)-1-[3-(difluoromethyl)-2-fluorophenyl]ethyl}-6-fluoro-2-methylpyrido[3,4-d]pyrimidin-4-amine

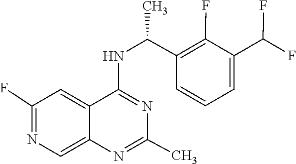 [SMILES: Cc1nc(N[C@H](C)c2cccc(C(F)F)c2F)c2cc(F)ncc2n1]

Using the method described for Example 1 using 6-fluoro-2-methylpyrido[3,4-d]pyrimidin-4-ol (200 mg, 1.12 mmol) and (1R)-1-[3-(difluoromethyl)-2-fluorophenyl]ethan-1-amine hydrochloride (302 mg, 1.34 mmol) gave the titled compound (187 mg, 45%) after preparative HPLC.

¹H-NMR (400 MHz, DMSO-d6) δ [ppm]: 1.604 (5.88), 1.621 (5.85), 2.392 (16.00), 2.518 (1.16), 2.522 (0.75), 5.741 (0.80), 5.758 (2.24), 5.775 (0.79), 7.102 (1.27), 7.238 (2.56), 7.278 (0.89), 7.297 (1.99), 7.316 (1.13), 7.374 (1.16), 7.497 (0.70), 7.514 (1.18), 7.532 (0.57), 7.667 (0.64), 7.685 (1.18), 7.704 (0.58), 8.152 (2.56), 8.735 (3.89), 8.796 (1.23), 8.814 (1.20).

### EXAMPLE 3

### Example 3: N-[(3R)-1-[4-[[(1R)-1-[3-(difluoromethyl)-2-fluoro-phenyl]ethyl]amino]-2-methyl-pyrido[3,4-d]pyrimidin-6-yl]pyrrolidin-3-yl]acetamide

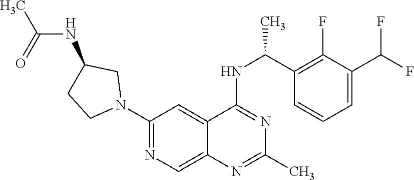 [SMILES: CC(=O)N[C@@H]1CCN(c2cc3c(N[C@H](C)c4cccc(C(F)F)c4F)nc(C)nc3cn2)C1]

To a solution of Example 2 (40 mg, 114 μmol) in DMSO (1.5 ml) was added N-[(3R)-pyrrolidin-3-yl]acetamide (58 mg, 457 μmol) and heated at 110° C. overnight. The reaction was purified by preparative HPLC (basic method) and gave the titled compound (41 mg, 74%).

¹H-NMR (400 MHz, DMSO-d6) δ [ppm]: 1.107 (1.65), 1.603 (4.46), 1.620 (4.45), 1.826 (16.00), 1.932 (0.54), 1.945 (0.57), 2.183 (0.44), 2.199 (0.54), 2.214 (0.48), 2.290 (13.85), 2.332 (0.42), 2.518 (2.11), 2.523 (1.31), 2.673 (0.42), 3.302 (2.03), 3.312 (2.35), 3.328 (2.85), 3.339 (2.94), 3.504 (0.49), 3.518 (0.51), 3.525 (0.60), 3.530 (0.68), 3.538 (0.58), 3.544 (0.72), 3.550 (0.65), 3.563 (0.51), 3.601 (0.46), 3.620 (0.93), 3.638 (0.54), 3.646 (0.65), 3.665 (0.96), 3.681 (0.95), 3.693 (0.82), 3.708 (0.73), 4.395 (0.58), 4.407 (0.58), 5.762 (0.66), 5.780 (1.02), 5.798 (0.66), 7.079 (2.80), 7.101 (1.04), 7.237 (2.19), 7.276 (0.77), 7.295 (1.67), 7.314 (0.97), 7.373 (0.90), 7.483 (0.56), 7.501 (0.96), 7.518 (0.47), 7.629 (0.52), 7.647 (0.96), 7.665 (0.47), 8.155 (4.92), 8.196 (1.14), 8.212 (1.13), 8.395 (1.08), 8.414 (1.04), 8.633 (4.17).

### EXAMPLE 4

### N-[(3S)-1-[4-[[(1R)-1-[3-(difluoromethyl)-2-fluoro-phenyl]ethyl]amino]-2-methyl-pyrido[3,4-d]pyrimidin-6-yl]pyrrolidin-3-yl]acetamide

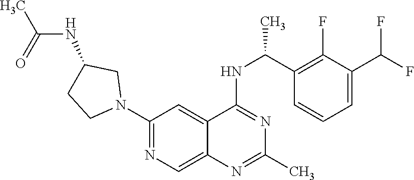 [SMILES: CC(=O)N[C@H]1CCN(c2cc3c(N[C@H](C)c4cccc(C(F)F)c4F)nc(C)nc3cn2)C1]

Using the method described for Example 3: Example 2 (40mg, 114 μmol) was treated with N-[(3S)-pyrrolidin-3-yl]acetamide (59 mg, 457 μmol) and gave the titled compound (41 mg, 75%).

¹H-NMR (400 MHz, DMSO-d6) δ [ppm]: 1.107 (3.59), 1.604 (4.53), 1.622 (4.52), 1.830 (16.00), 1.929 (0.56), 1.943 (0.58), 1.960 (0.42), 2.180 (0.46), 2.197 (0.55), 2.212 (0.49), 2.288 (14.00), 2.518 (1.56), 2.523 (0.97), 3.548 (0.41), 3.554 (0.59), 3.561 (0.43), 3.568 (0.67), 3.574 (0.63), 3.587 (0.86), 3.606 (0.98), 3.624 (0.53), 3.632 (0.54), 3.645 (0.83), 3.661 (0.93), 3.672 (0.79), 3.687 (0.71), 4.400 (0.58), 4.413 (0.58), 5.757 (0.70), 5.775 (1.05), 5.793 (0.67), 7.074 (2.82), 7.100 (1.06), 7.237 (2.21), 7.274 (0.78), 7.293 (1.70), 7.312 (0.99), 7.372 (0.91), 7.483 (0.57), 7.499 (0.98), 7.517 (0.48), 7.626 (0.52), 7.644 (0.96), 7.661 (0.48), 8.202 (1.23), 8.208 (0.94), 8.219 (1.21), 8.396 (1.15), 8.414 (1.11), 8.633 (4.25).

### EXAMPLE 5

### N-[(1R)-1-[3-(difluoromethyl)-2-fluoro-phenyl]ethyl]-2-methyl-6-pyrrolidin-1-yl-pyrido[3,4-d]pyrimidin-4-amine

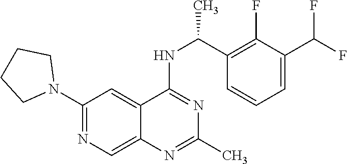 [SMILES: Cc1nc(N[C@H](C)c2cccc(C(F)F)c2F)c2cc(N3CCCC3)ncc2n1]

Using the method described for Example 3: Example 2 (40mg, 114 μmol) was treated with pyrrolidine (32 mg, 457 μmol) and gave the titled compound (42 mg, 86%).

¹H-NMR (400 MHz, DMSO-d6) δ [ppm]: 1.107 (13.90), 1.604 (5.13), 1.622 (5.13), 1.990 (1.90), 2.000 (2.33), 2.007 (5.52), 2.014 (2.31), 2.023 (1.97), 2.285 (16.00), 2.518 (1.84), 2.522 (1.19), 3.448 (0.59), 3.457 (1.23), 3.473 (3.14), 3.481 (3.09), 3.497 (1.14), 3.506 (0.55), 5.762 (0.77), 5.780 (1.18), 5.798 (0.74), 7.061 (3.26), 7.100 (1.17), 7.236 (2.42), 7.270 (0.85), 7.289 (1.85), 7.308 (1.07), 7.372 (1.02), 7.481 (0.63), 7.498 (1.06), 7.516 (0.51), 7.634 (0.58), 7.652 (1.05), 7.670 (0.52), 8.362 (1.27), 8.380 (1.22), 8.621 (4.54).

### EXAMPLE 6

### N-{(1R)-1-[3-(difluoromethyl)-2-methylphenyl]ethyl}-6-fluoro-2-methylpyrido[3,4-d]pyrimidin-4-amine

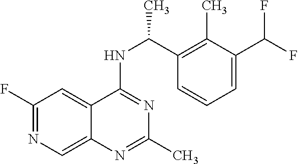 [SMILES: Cc1nc(N[C@H](C)c2cccc(C(F)F)c2C)c2cc(F)ncc2n1]

Using the method described for Example 1 using 6-fluoro-2-methylpyrido[3,4-d]pyrimidin-4-ol and (1R)-1-[3-(difluoromethyl)-2-methylphenyl]ethan-1-amine hydrochloride gave the titled compound.

¹H-NMR (400 MHz, DMSO-d6) δ [ppm]: 1.542 (5.50), 1.560 (5.58), 2.401 (16.00), 2.518 (1.42), 2.523 (1.00), 2.543 (8.10), 5.706 (0.82), 5.723 (1.27), 5.741 (0.81), 7.079 (1.03), 7.216 (2.15), 7.278 (0.70), 7.297 (1.70), 7.317 (1.11), 7.353 (0.92), 7.388 (1.66), 7.407 (1.12), 7.637 (1.34), 7.656 (1.19), 8.143 (2.44), 8.145 (2.48), 8.711 (4.26), 8.828 (1.24), 8.846 (1.20).

### EXAMPLE 7

### N-[(3R)-1-[4-[[(1R)-1-[3-(difluoromethyl)-2-methyl-phenyl]ethyl]amino]-2-methyl-pyrido[3,4-d]pyrimidin-6-yl]pyrrolidin-3-yl]acetamide

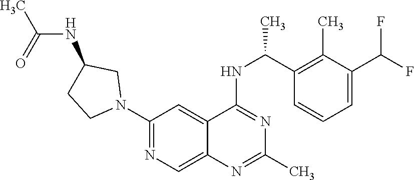 [SMILES: CC(=O)N[C@@H]1CCN(c2cc3c(N[C@H](C)c4cccc(C(F)F)c4C)nc(C)nc3cn2)C1]

Using the method described for Example 3: Example 6 was treated with N-[(3R)-pyrrolidin-3-yl]acetamide and gave the titled compound.

¹H-NMR (400 MHz, DMSO-d6) δ [ppm]: 1.539 (3.55), 1.556 (3.58), 1.825 (13.28), 1.928 (0.46), 1.942 (0.48), 2.195 (0.47), 2.210 (0.41), 2.303 (11.17), 2.323 (0.45), 2.327 (0.57), 2.518 (2.39), 2.523 (1.71), 2.540 (16.00), 2.669 (0.52), 3.300 (0.57), 3.310 (0.66), 3.523 (0.45), 3.536 (0.47), 3.542 (0.42), 3.613 (0.72), 3.631 (0.40), 3.638 (0.50), 3.658 (0.77), 3.674 (0.76), 3.685 (0.64), 3.700 (0.57), 4.391 (0.49), 4.405 (0.48), 5.720 (0.55), 5.738 (0.84), 5.756 (0.54), 7.069 (2.40), 7.075 (0.97), 7.214 (1.47), 7.277 (0.52), 7.296 (1.27), 7.315 (0.84), 7.351 (0.62), 7.376 (1.27), 7.393 (0.82), 7.630 (1.01), 7.649 (0.89), 8.192 (0.97), 8.208 (0.96), 8.434 (0.95), 8.453 (0.91), 8.610 (3.38).

### EXAMPLE 8

### N-[(3S)-1-[4-[[(1R)-1-[3-(difluoromethyl)-2-methyl-phenyl]ethyl]amino]-2-methyl-pyrido[3,4-d]pyrimidin-6-yl]pyrrolidin-3-yl]acetamide

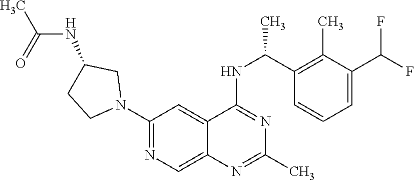 [SMILES: CC(=O)N[C@H]1CCN(c2cc3c(N[C@H](C)c4cccc(C(F)F)c4C)nc(C)nc3cn2)C1]

Using the method described for Example 3: Example 6 was treated with N-[(3S)-pyrrolidin-3-yl]acetamide and gave the titled compound.

¹H-NMR (400 MHz, DMSO-d6) δ [ppm]: 1.539 (4.28), 1.557 (4.28), 1.829 (16.00), 1.925 (0.58), 1.938 (0.59), 1.956 (0.41), 2.175 (0.46), 2.191 (0.55), 2.206 (0.49), 2.303 (13.86), 2.323 (0.54), 2.327 (0.69), 2.331 (0.49), 2.518 (2.77), 2.523 (1.97), 2.541 (6.90), 2.665 (0.47), 2.669 (0.63), 2.673 (0.44), 3.308 (0.74), 3.319 (0.95), 3.345 (0.98), 3.550 (0.57), 3.564 (0.65), 3.570 (0.63), 3.582 (0.75), 3.599 (0.98), 3.616 (0.51), 3.625 (0.50), 3.638 (0.81), 3.654 (0.91), 3.665 (0.76), 3.680 (0.67), 4.400 (0.59), 4.413 (0.57), 5.716 (0.66), 5.734 (1.01), 5.751 (0.65), 7.063 (2.77), 7.076 (0.90), 7.214 (1.77), 7.275 (0.64), 7.294 (1.53), 7.313 (1.04), 7.351 (0.73), 7.375 (1.50), 7.393 (0.97), 7.627 (1.19), 7.646 (1.06), 8.199 (1.19), 8.216 (1.15), 8.435 (1.12), 8.453 (1.07), 8.610 (4.01).

### EXAMPLE 9

### N-[(1R)-1-[3-(difluoromethyl)-2-methyl-phenyl]ethyl]-2-methyl-6-pyrrolidin-1-yl-pyrido[3,4-d]pyrimidin-4-amine

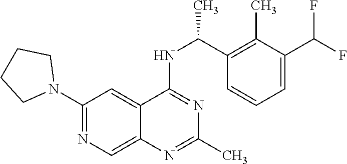 [SMILES: Cc1nc(N[C@H](C)c2cccc(C(F)F)c2C)c2cc(N3CCCC3)ncc2n1]

Using the method described for Example 3: Example 6 was treated with pyrrolidine and gave the titled compound.

¹H-NMR (400 MHz, DMSO-d6) δ [ppm]: 1.541 (5.03), 1.558 (4.97), 1.986 (1.89), 1.996 (2.29), 2.003 (5.67), 2.010 (2.30), 2.019 (1.99), 2.299 (16.00), 2.322 (0.61), 2.326 (0.81), 2.332 (0.59), 2.518 (3.54), 2.523 (2.38), 2.539 (7.68), 2.664 (0.57), 2.669 (0.80), 2.673 (0.58), 3.441 (0.58), 3.450 (1.11), 3.466 (3.01), 3.476 (3.00), 3.483 (1.78), 3.492 (1.08), 3.502 (0.57), 5.720 (0.73), 5.739 (1.14), 5.756 (0.73), 7.051 (3.18), 7.075 (0.97), 7.212 (2.01), 7.271 (0.67), 7.290 (1.65), 7.310 (1.10), 7.350 (0.84), 7.374 (1.62), 7.392 (1.06), 7.640 (1.28), 7.660 (1.14), 8.396 (1.23), 8.414 (1.19), 8.599 (4.28).

### EXAMPLE 10

### 6-fluoro-2-methyl-N-[(1R)-1-[3-(trifluoromethyl)phenyl]ethyl]pyrido[3,4-d]pyrimidin-4-amine

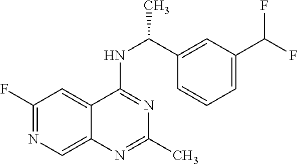 [SMILES: Cc1nc(N[C@H](C)c2cccc(C(F)F)c2)c2cc(F)ncc2n1]

Using the method described for Example 1 using 6-fluoro-2-methylpyrido[3,4-d]pyrimidin-4-ol and (1R)-1-[3-(trifluoromethyl)phenyl]ethan-1-amine hydrochloride gave the titled compound.

¹H-NMR (400 MHz, DMSO-d6) δ [ppm]: 1.612 (5.87), 1.629 (5.90), 1.986 (0.64), 2.421 (16.00), 2.518 (1.36), 2.523 (0.91), 5.603 (0.79), 5.620 (1.18), 5.639 (0.77), 7.550 (0.50), 7.569 (1.59), 7.588 (1.81), 7.598 (2.01), 7.617 (0.61), 7.752 (1.37), 7.770 (1.08), 7.830 (2.25), 8.098 (2.53), 8.101 (2.53), 8.731 (4.14), 8.765 (1.20), 8.784 (1.16).

### EXAMPLE 11

### N-[(3R)-1-[2-methyl-4-[[(1R)-1-[3-(trifluoromethyl)phenyl]ethyl]amino]pyrido[3,4-d]pyrimidin-6-yl]pyrrolidin-3-yl]acetamide

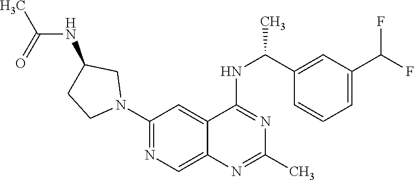 [SMILES: CC(=O)N[C@@H]1CCN(c2cc3c(N[C@H](C)c4cccc(C(F)F)c4)nc(C)nc3cn2)C1]

Using the method described for Example 3: Example 10 was treated with N-[(3R)-pyrrolidin-3-yl]acetamide and gave the titled compound.

¹H-NMR (400 MHz, DMSO-d6) δ [ppm]: 1.613 (4.68), 1.631 (4.71), 1.823 (16.00), 1.927 (0.53), 1.940 (0.56), 2.179 (0.44), 2.195 (0.52), 2.210 (0.46), 2.321 (14.30), 2.332 (0.69), 2.518 (1.70), 2.523 (1.16), 2.669 (0.57), 3.294 (0.64), 3.303 (0.69), 3.519 (0.40), 3.525 (0.50), 3.532 (0.40), 3.538 (0.55), 3.545 (0.49), 3.610 (0.90), 3.628 (0.48), 3.636 (0.59), 3.655 (0.91), 3.670 (0.88), 3.682 (0.76), 3.698 (0.67), 4.390 (0.56), 4.404 (0.55), 5.623 (0.67), 5.642 (0.98), 5.659 (0.64), 7.033 (2.77), 7.569 (1.35), 7.589 (2.87), 7.732 (1.17), 7.750 (0.87), 7.797 (1.99), 8.191 (1.11), 8.208 (1.09), 8.377 (1.14), 8.397 (1.10), 8.633 (4.11).

### EXAMPLE 12

### N-[(3S)-1-[2-methyl-4-[[(1R)-1-[3-(trifluoromethyl)phenyl]ethyl]amino]pyrido[3,4-d]pyrimidin-6-yl]pyrrolidin-3-yl]acetamide

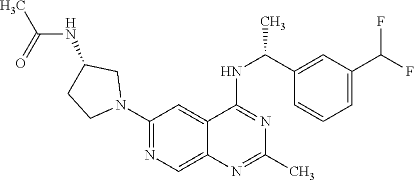 [SMILES: CC(=O)N[C@H]1CCN(c2cc3c(N[C@H](C)c4cccc(C(F)F)c4)nc(C)nc3cn2)C1]

Using the method described for Example 3: Example 10 was treated with N-[(3S)-pyrrolidin-3-yl]acetamide and gave the titled compound.

¹H-NMR (400 MHz, DMSO-d6) δ [ppm]: 1.615 (4.44), 1.633 (4.45), 1.824 (16.00), 1.924 (0.53), 1.938 (0.54), 2.176 (0.43), 2.193 (0.51), 2.207 (0.44), 2.320 (13.83), 2.332 (0.62), 2.518 (1.81), 2.523 (1.17), 2.540 (5.21), 2.669 (0.52), 3.304 (0.67), 3.315 (0.78), 3.546 (0.53), 3.559 (0.59), 3.565 (0.55), 3.580 (0.68), 3.599 (0.90), 3.618 (0.48), 3.625 (0.51), 3.636 (0.76), 3.651 (0.87), 3.662 (0.73), 3.678 (0.64), 4.395 (0.54), 4.409 (0.53), 5.618 (0.63), 5.636 (0.93), 5.654 (0.61), 7.027 (2.64), 7.568 (1.28), 7.587 (2.70), 7.592 (1.40), 7.732 (1.12), 7.749 (0.83), 7.795 (1.87), 8.196 (1.09), 8.213 (1.08), 8.376 (1.13), 8.395 (1.07), 8.634 (3.91).

### EXAMPLE 13

### N-[(1R)-1-[3-(1,1-difluoroethyl)phenyl]ethyl]-6-fluoro-2-methyl-pyrido[3,4-d]pyrimidin-4-amine

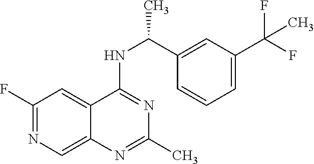 [SMILES: Cc1nc(N[C@H](C)c2cccc(C(C)(F)F)c2)c2cc(F)ncc2n1]

Using the method described for Example 1 using 6-fluoro-2-methylpyrido[3,4-d]pyrimidin-4-ol and (1R)-1-[3-(1,1-difluoroethyl)phenyl]ethanamine hydrochloride gave the titled compound.

¹H-NMR (400 MHz, DMSO-d6) δ [ppm]: 1.600 (5.73), 1.618 (5.72), 1.910 (4.85), 1.957 (9.96), 2.004 (4.33), 2.435 (16.00), 2.518 (2.83), 2.523 (1.94), 2.673 (0.48), 5.596 (0.56), 5.614 (0.85), 5.632 (0.55), 7.436 (3.99), 7.442 (1.34), 7.451 (1.86), 7.471 (0.45), 7.567 (1.06), 7.579 (0.73), 7.583 (0.95), 7.672 (2.13), 8.110 (2.28), 8.113 (2.30), 8.726 (3.70), 8.738 (0.94), 8.756 (0.86).

### EXAMPLE 14

### N-[(3R)-1-[4-[[(1R)-1-[3-(1,1-difluoroethyl)phenyl]ethyl]amino]-2-methyl-pyrido[3,4-d]pyrimidin-6-yl]pyrrolidin-3-yl]acetamide

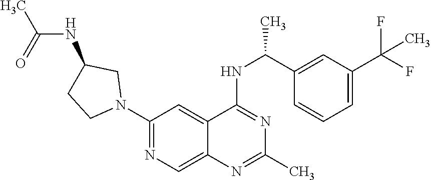 [SMILES: CC(=O)N[C@@H]1CCN(c2cc3c(N[C@H](C)c4cccc(C(C)(F)F)c4)nc(C)nc3cn2)C1]

Using the method described for Example 3: Example 13 was treated with N-[(3R)-pyrrolidin-3-yl]acetamide and gave the titled compound.

¹H-NMR (400 MHz, DMSO-d6) δ [ppm]: 1.600 (4.60), 1.618 (4.63), 1.822 (16.00), 1.906 (4.45), 1.924 (0.60), 1.937 (0.69), 1.953 (8.68), 2.000 (3.68), 2.175 (0.50), 2.192 (0.57), 2.207 (0.51), 2.223 (0.41), 2.323 (1.11), 2.333 (14.89), 2.518 (4.14), 2.523 (2.81), 2.665 (0.69), 2.669 (0.96), 2.673 (0.68), 3.291 (0.57), 3.301 (0.75), 3.318 (0.95), 3.516 (0.41), 3.522 (0.48), 3.535 (0.57), 3.542 (0.56), 3.555 (0.47), 3.606 (0.81), 3.614 (0.47), 3.624 (0.51), 3.632 (0.68), 3.649 (0.93), 3.664 (0.81), 3.676 (0.77), 3.692 (0.60), 4.391 (0.60), 4.403 (0.59), 5.619 (0.60), 5.637 (0.87), 5.656 (0.57), 7.042 (2.54), 7.407 (0.45), 7.429 (2.75), 7.447 (1.71), 7.466 (0.59), 7.547 (1.22), 7.564 (0.99), 7.651 (2.18), 8.191 (1.10), 8.208 (1.10), 8.353 (1.23), 8.372 (1.17), 8.628 (4.36).

### EXAMPLE 15

### N-[(3S)-1-[4-[[(1R)-1-[3-(1,1-difluoroethyl)phenyl]ethyl]amino]-2-methyl-pyrido[3,4-d]pyrimidin-6-yl]pyrrolidin-3-yl]acetamide

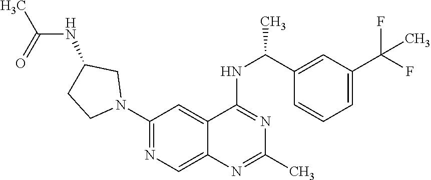 [SMILES: CC(=O)N[C@H]1CCN(c2cc3c(N[C@H](C)c4cccc(C(C)(F)F)c4)nc(C)nc3cn2)C1]

Using the method described for Example 3: Example 13 was treated with N-[(3S)-pyrrolidin-3-yl]acetamide and gave the titled compound.

¹H-NMR (400 MHz, DMSO-d6) δ [ppm]: 1.602 (4.60), 1.619 (4.59), 1.822 (16.00), 1.904 (4.38), 1.921 (0.62), 1.935 (0.66), 1.952 (8.60), 1.998 (3.64), 2.173 (0.54), 2.190 (0.59), 2.206 (0.51), 2.333 (14.50), 2.518 (3.18), 2.523 (2.14), 2.665 (0.53), 2.669 (0.73), 2.673 (0.52), 3.301 (0.73), 3.311 (0.72), 3.535 (0.46), 3.541 (0.59), 3.554 (0.62), 3.561 (0.52), 3.575 (0.53), 3.597 (0.82), 3.605 (0.47), 3.615 (0.53), 3.623 (0.56), 3.631 (0.81), 3.647 (0.92), 3.658 (0.70), 3.674 (0.69), 4.393 (0.61), 4.405 (0.60), 5.614 (0.59), 5.632 (0.88), 5.651 (0.60), 7.038 (2.54), 7.406 (0.46), 7.428 (2.78), 7.446 (1.74), 7.466 (0.59), 7.547 (1.21), 7.564 (0.99), 7.650 (2.20), 8.195 (1.12), 8.211 (1.10), 8.352 (1.22), 8.372 (1.17), 8.628 (4.42).

### EXAMPLE 16

### N-[(1R)-1-[3-(1,1-difluoroethyl)phenyl]ethyl]-6-fluoro-2-methyl-pyrido[3,4-d]pyrimidin-4-amine

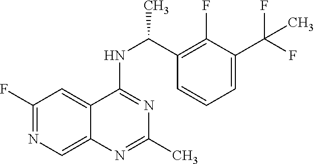 [SMILES: Cc1nc(N[C@H](C)c2cccc(C(C)(F)F)c2F)c2cc(F)ncc2n1]

Using the method described for Example 1 using 6-fluoro-2-methylpyrido[3,4-d]pyrimidin-4-ol and (1R)-1-[3-(1,1-difluoroethyl)-2-fluoro-phenyl]ethanamine hydrochloride gave the titled compound.

¹H-NMR (400 MHz, DMSO-d6) δ [ppm]: 1.600 (5.37), 1.617 (5.29), 1.981 (2.65), 2.028 (5.15), 2.076 (2.38), 2.332 (0.68), 2.392 (16.00), 2.518 (3.61), 2.523 (2.50), 2.673 (0.68), 5.735 (0.79), 5.753 (1.23), 5.770 (0.79), 7.235 (0.82), 7.254 (1.84), 7.273 (1.08), 7.429 (0.66), 7.447 (1.11), 7.463 (0.56), 7.621 (0.59), 7.637 (1.05), 7.655 (0.52), 8.150 (2.33), 8.153 (2.33), 8.735 (3.90), 8.792 (1.14), 8.810 (1.11).

### EXAMPLE 17

### N-[(3R)-1-[4-[[(1R)-1-[3-(1,1-difluoroethyl)-2-fluoro-phenyl]ethyl]amino]-2-methyl-pyrido[3,4-d]pyrimidin-6-yl]pyrrolidin-3-yl]acetamide

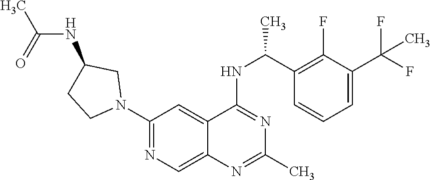 [SMILES: CC(=O)N[C@@H]1CCN(c2cc3c(N[C@H](C)c4cccc(C(C)(F)F)c4F)nc(C)nc3cn2)C1]

Using the method described for Example 3: Example 16 was treated with N-[(3R)-pyrrolidin-3-yl]acetamide and gave the titled compound.

¹H-NMR (400 MHz, DMSO-d6) δ [ppm]: 1.597 (4.59), 1.615 (4.59), 1.827 (16.00), 1.932 (0.59), 1.945 (0.64), 1.963 (0.51), 1.981 (2.75), 2.029 (5.16), 2.077 (2.35), 2.183 (0.54), 2.199 (0.60), 2.214 (0.54), 2.232 (0.43), 2.289 (13.51), 2.518 (4.05), 2.523 (2.72), 3.301 (0.73), 3.312 (0.87), 3.526 (0.44), 3.532 (0.56), 3.545 (0.60), 3.551 (0.52), 3.621 (0.89), 3.639 (0.51), 3.647 (0.62), 3.667 (0.92), 3.683 (0.94), 3.694 (0.81), 3.710 (0.71), 4.395 (0.62), 4.408 (0.60), 5.755 (0.73), 5.773 (1.11), 5.791 (0.70), 7.079 (2.91), 7.231 (0.83), 7.250 (1.80), 7.269 (1.05), 7.413 (0.67), 7.431 (1.08), 7.447 (0.51), 7.583 (0.59), 7.600 (1.05), 7.617 (0.52), 8.198 (1.22), 8.214 (1.18), 8.396 (1.18), 8.414 (1.13), 8.630 (4.27).

### EXAMPLE 18

N-[(3S)-1-[4-[[(1R)-1-[3-(1,1-difluoroethyl)-2-fluoro-phenyl]ethyl]amino]-2-methyl-pyrido[3,4-d]pyrimidin-6-yl]pyrrolidin-3-yl]acetamide

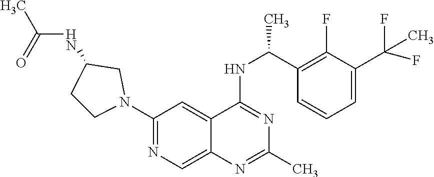 [SMILES: CC(=O)N[C@H]1CCN(c2cc3c(N[C@H](C)c4cccc(C(C)(F)F)c4F)nc(C)nc3cn2)C1]

Using the method described for Example 3: Example 16 was treated with N-[(3S)-pyrrolidin-3-yl]acetamide and gave the titled compound.

¹H-NMR (400 MHz, DMSO-d6) δ [ppm]: 1.514 (0.42), 1.598 (4.69), 1.616 (4.66), 1.830 (16.00), 1.912 (0.41), 1.930 (0.64), 1.944 (0.67), 1.960 (0.65), 1.981 (2.81), 2.007 (0.47), 2.029 (5.36), 2.076 (2.45), 2.182 (0.56), 2.197 (0.67), 2.216 (0.92), 2.229 (0.52), 2.287 (13.61), 2.318 (0.44), 2.323 (0.80), 2.327 (1.08), 2.331 (0.78), 2.518 (3.87), 2.523 (2.53), 2.665 (0.67), 2.669 (0.95), 2.673 (0.64), 3.315 (1.05), 3.352 (0.80), 3.556 (0.59), 3.570 (0.69), 3.576 (0.64), 3.589 (0.87), 3.606 (1.03), 3.624 (0.54), 3.632 (0.52), 3.646 (0.85), 3.662 (0.95), 3.673 (0.80), 3.688 (0.70), 4.401 (0.62), 4.413 (0.60), 5.750 (0.74), 5.767 (1.13), 5.785 (0.70), 7.074 (2.93), 7.229 (0.85), 7.248 (1.85), 7.267 (1.11), 7.412 (0.67), 7.430 (1.09), 7.447 (0.52), 7.580 (0.62), 7.596 (1.06), 7.614 (0.52), 8.203 (1.26), 8.220 (1.21), 8.396 (1.21), 8.414 (1.14), 8.631 (4.33).

### EXAMPLE 19

### N-{(1R)-1-[3-(difluoromethyl)-2-fluorophenyl]ethyl}-6-fluoro-2,8-dimethylpyrido[3,4-d]pyrimidin-4-amine

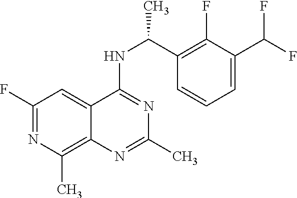 [SMILES: Cc1nc(N[C@H](C)c2cccc(C(F)F)c2F)c2cc(F)nc(C)c2n1]

To a solution of Example 2 (250 mg, 714 μmol) in DMSO (5 ml) was added DBU (213 μl, 3.6 mmol) and nitromethane (193 μl, 1.43 mmol) and stirred for 4 days at RT. The reaction was diluted with water and the solid collected by filtration and washed with water. The solid was dried to give the title compound (261 mg, 95%).

¹H-NMR (600 MHz, DMSO-d6) δ [ppm]: 0.909 (0.44), 1.111 (2.03), 1.233 (0.43), 1.601 (6.17), 1.612 (5.96), 2.386 (0.69), 2.388 (0.89), 2.391 (0.77), 2.395 (0.65), 2.403 (16.00), 2.519 (1.95), 2.522 (1.82), 2.525 (1.44), 2.613 (0.46), 2.616 (0.66), 2.619 (0.53), 2.727 (12.15), 3.313 (0.74), 5.757 (0.60), 7.142 (1.06), 7.232 (2.12), 7.276 (0.96), 7.289 (2.03), 7.302 (1.12), 7.323 (0.94), 7.496 (0.63), 7.508 (1.10), 7.519 (0.57), 7.658 (0.60), 7.669 (1.12), 7.681 (0.57), 7.949 (2.43), 8.088 (0.78), 8.316 (4.63), 8.693 (0.48).

### EXAMPLE 20

### N-{(3R)-1-[4-({(1R)-1-[3-(difluoromethyl)-2-fluorophenyl]ethyl}amino)-2,8-dimethylpyrido[3,4-d]pyrimidin-6-yl]pyrrolidin-3-yl}acetamide

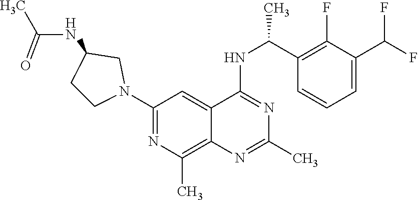 [SMILES: CC(=O)N[C@@H]1CCN(c2cc3c(N[C@H](C)c4cccc(C(F)F)c4F)nc(C)nc3c(C)n2)C1]

To a solution of Example 19 (20.8 mg, 57 μmol) in DMSO (0.5 ml) was added N-[(3R)-pyrrolidin-3-yl]acetamide (14 mg, 114 μmol) and TEA (32 μl, 228 μmol). The reaction was heated at 110° C. for 16 h. The reaction was allowed to cool and then purified by preparative HPLC (basic method) to give the titled compound (9.5 mg, 35%).

¹H-NMR (400 MHz, DMSO-d6) δ [ppm]: 1.094 (3.50), 1.170 (0.41), 1.228 (1.01), 1.591 (5.83), 1.608 (6.19), 1.820 (16.00), 1.903 (1.21), 1.913 (0.98), 1.927 (0.99), 1.944 (0.74), 2.164 (0.77), 2.179 (0.98), 2.195 (0.91), 2.211 (0.68), 2.297 (12.97), 2.323 (1.19), 2.637 (13.81), 2.657 (1.41), 2.665 (1.17), 3.286 (1.41), 3.297 (1.92), 3.478 (0.48), 3.503 (0.95), 3.517 (1.03), 3.536 (0.66), 3.589 (0.59), 3.606 (1.24), 3.624 (0.90), 3.631 (1.01), 3.655 (1.28), 3.670 (1.35), 3.682 (1.17), 3.698 (1.01), 4.352 (0.62), 4.366 (1.06), 4.379 (1.05), 5.753 (0.98), 5.770 (1.50), 5.788 (1.01), 6.896 (3.50), 7.095 (1.23), 7.231 (2.49), 7.264 (1.09), 7.283 (2.32), 7.302 (1.39), 7.367 (1.13), 7.473 (0.99), 7.490 (1.63), 7.507 (0.88), 7.614 (0.92), 7.632 (1.64), 7.649 (0.87), 8.084 (0.50), 8.172 (1.75), 8.188 (1.72), 8.275 (1.03), 8.292 (1.07).

### EXAMPLE 21

### N-{(1R)-1-[3-(difluoromethyl)-2-fluorophenyl]ethyl}-6-[(3R)-3-(dimethylamino)pyrrolidin-1-yl]-2,8-dimethylpyrido[3,4-d]pyrimidin-4-amine

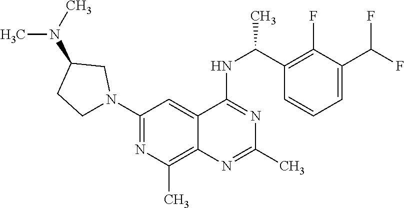 [SMILES: Cc1nc(N[C@H](C)c2cccc(C(F)F)c2F)c2cc(N3CC[C@@H](N(C)C)C3)nc(C)c2n1]

Using the method described for Example 20: Example 19 was treated with (3R)-N,N-dimethylpyrrolidin-3-amine (58.0 mg, 508 μmol) and gave the titled compound (25 mg, 51%) after preparative HPLC (basic method).

¹H-NMR (400 MHz, DMSO-d6) δ [ppm]: 1.602 (3.56), 1.619 (3.58), 1.854 (0.51), 1.876 (0.42), 2.182 (0.49), 2.197 (0.49), 2.239 (16.00), 2.296 (9.36), 2.323 (0.67), 2.327 (0.82), 2.639 (8.90), 2.665 (0.68), 2.669 (0.81), 2.812 (0.40), 2.830 (0.53), 3.153 (0.62), 3.178 (0.80), 3.198 (0.59), 3.383 (0.67), 3.400 (0.64), 3.645 (0.43), 3.666 (0.70), 3.742 (0.56), 3.759 (0.67), 3.766 (0.64), 3.784 (0.48), 5.757 (0.56), 5.775 (0.86), 5.793 (0.54), 6.865 (2.29), 7.100 (0.76), 7.236 (1.58), 7.262 (0.61), 7.282 (1.35), 7.301 (0.80), 7.371 (0.70), 7.474 (0.53), 7.491 (0.87), 7.509 (0.44), 7.619 (0.48), 7.638 (0.87), 7.655 (0.45), 8.217 (0.95), 8.235 (0.94).

### EXAMPLE 22

### 1-{4-[4-({(1R)-1-[3-(difluoromethyl)-2-fluorophenyl]ethyl}amino)-2,8-dimethylpyrido[3,4-d]pyrimidin-6-yl]piperazin-1-yl}ethan-1-one

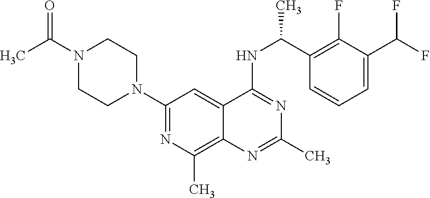 [SMILES: CC(=O)N1CCN(c2cc3c(N[C@H](C)c4cccc(C(F)F)c4F)nc(C)nc3c(C)n2)CC1]

Using the method described for Example 20: Example 19 was treated with 1-(piperazin-1-yl)ethan-1-one (65.1 mg, 508 μmol) and gave the titled compound (20 mg, 40%) after preparative HPLC (basic method).

¹H-NMR (400 MHz, DMSO-d6) δ [ppm]: 0.967 (0.44), 1.107 (0.42), 1.603 (5.55), 1.621 (5.45), 1.957 (0.40), 2.074 (16.00), 2.321 (15.94), 2.432 (0.46), 2.522 (4.88), 2.658 (14.29), 2.669 (1.88), 3.516 (1.82), 3.606 (9.65), 5.749 (0.91), 5.766 (1.31), 5.784 (0.82), 7.101 (1.29), 7.238 (2.64), 7.272 (0.97), 7.293 (4.58), 7.310 (1.29), 7.374 (1.12), 7.485 (0.72), 7.500 (1.25), 7.517 (0.63), 7.622 (0.70), 7.641 (1.22), 7.658 (0.63), 8.340 (1.37), 8.359 (1.35).

### EXAMPLE 23

### N-{(1R)-1-[3-(difluoromethyl)-2-fluorophenyl]ethyl}-2,8-dimethyl-6-(4-methylpiperazin-1-yl)pyrido[3,4-d]pyrimidin-4-amine

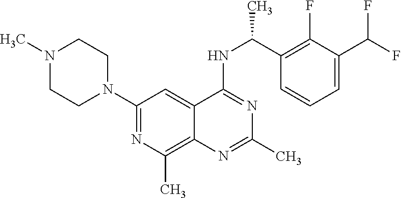 [SMILES: Cc1nc(N[C@H](C)c2cccc(C(F)F)c2F)c2cc(N3CCN(C)CC3)nc(C)c2n1]

Using the method described for Example 20: Example 19 was treated with 1-methylpiperazine (110 μl, 1.0 mmol) and gave the titled compound (30 mg, 60%) after preparative HPLC (basic method).

¹H-NMR (400 MHz, DMSO-d6) δ [ppm]: 0.860 (0.75), 0.967 (2.61), 1.109 (1.08), 1.144 (1.52), 1.209 (0.57), 1.224 (0.66), 1.596 (5.12), 1.614 (5.11), 2.252 (10.44), 2.313 (16.00), 2.322 (1.22), 2.327 (1.13), 2.332 (0.78), 2.459 (2.46), 2.471 (3.88), 2.518 (3.87), 2.523 (2.45), 2.642 (13.95), 2.660 (0.42), 2.665 (0.72), 2.669 (0.97), 2.673 (0.71), 3.525 (2.34), 3.537 (3.14), 3.549 (2.33), 5.744 (0.78), 5.762 (1.20), 5.780 (0.77), 7.101 (1.16), 7.237 (2.68), 7.245 (3.17), 7.269 (0.89), 7.289 (1.92), 7.307 (1.11), 7.373 (1.02), 7.480 (0.65), 7.497 (1.10), 7.514 (0.54), 7.620 (0.59), 7.637 (1.08), 7.655 (0.53), 8.313 (1.29), 8.331 (1.24).

### EXAMPLE 24

### 2-[4-({(1R)-1-[3-(difluoromethyl)-2-fluorophenyl]ethyl}amino)-2,8-dimethylpyrido[3,4-d]pyrimidin-6-yl]-2,6-diazaspiro[3.4]octan-7-one

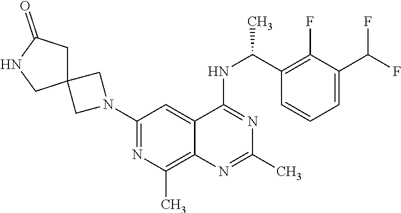 [SMILES: Cc1nc(N[C@H](C)c2cccc(C(F)F)c2F)c2cc(N3CC4(CNC(=O)C4)C3)nc(C)c2n1]

Using the method described for Example 20: Example 19 was treated with 2,6-diazaspiro[3.4]octan-7-one (64.1 mg, 508 μmol) and gave the titled compound (20 mg, 40%) after preparative HPLC (basic method).

¹H-NMR (400 MHz, DMSO-d6) δ [ppm]: 1.109 (0.49), 1.231 (0.52), 1.348 (0.42), 1.569 (0.44), 1.587 (5.53), 1.605 (5.56), 2.286 (0.50), 2.310 (16.00), 2.322 (1.35), 2.327 (1.54), 2.332 (1.12), 2.422 (0.79), 2.428 (0.51), 2.432 (0.74), 2.449 (0.49), 2.518 (5.90), 2.523 (3.76), 2.542 (8.45), 2.632 (13.73), 2.660 (0.51), 2.665 (1.05), 2.669 (1.45), 2.673 (1.05), 2.678 (0.49), 3.522 (6.98), 3.954 (0.86), 3.978 (8.61), 4.003 (0.84), 5.738 (0.91), 5.756 (1.33), 5.774 (0.83), 6.966 (3.70), 7.097 (1.28), 7.233 (2.68), 7.265 (0.95), 7.285 (2.08), 7.303 (1.25), 7.369 (1.15), 7.478 (0.74), 7.495 (1.24), 7.513 (0.64), 7.617 (0.69), 7.634 (1.30), 7.653 (0.69), 7.676 (2.61), 8.088 (0.56), 8.299 (1.40), 8.317 (1.35).

### EXAMPLE 25

### N-{(3S)-1-[4-({(1R)-1-[3-(difluoromethyl)phenyl]ethyl}amino)-2-methylpyrido[3,4-d]pyrimidin-6-yl]pyrrolidin-3-yl}acetamide

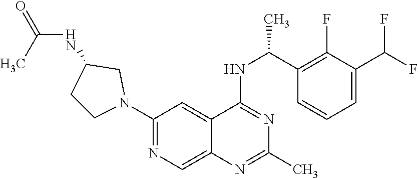 [SMILES: CC(=O)N[C@H]1CCN(c2cc3c(N[C@H](C)c4cccc(C(F)F)c4F)nc(C)nc3cn2)C1]

To a solution of intermediate 10 (57.7 mg, 201 μmol) and PyBOP (136 mg, 261 μmol) in DMF (580 μL) was added DBU (90 μl, 600 μmol) followed by (1R)-1-[3-(difluoromethyl)phenyl]ethan-1-amine hydrochloride (50.0 mg, 241 μmol). The reaction was stirred at RT for 16 h. The titled compound was isolated (50 mg, 54%) after preparative HPLC purification (basic method).

¹H-NMR (400 MHz, DMSO-d6) δ [ppm]: 1.601 (4.90), 1.619 (4.90), 1.823 (16.00), 1.922 (0.60), 1.935 (0.61), 1.953 (0.44), 2.174 (0.49), 2.190 (0.58), 2.205 (0.52), 2.221 (0.40), 2.326 (14.40), 2.518 (1.83), 2.522 (1.14), 2.669 (0.42), 3.303 (0.74), 3.313 (0.85), 3.542 (0.59), 3.550 (0.43), 3.556 (0.67), 3.563 (0.62), 3.577 (0.71), 3.597 (1.00), 3.615 (0.54), 3.623 (0.59), 3.632 (0.84), 3.648 (0.99), 3.658 (0.81), 3.674 (0.72), 4.394 (0.62), 4.406 (0.61), 5.623 (0.67), 5.641 (1.01), 5.659 (0.66), 6.884 (1.24), 7.024 (2.60), 7.045 (2.98), 7.164 (1.15), 7.415 (0.75), 7.434 (1.54), 7.459 (1.15), 7.478 (1.73), 7.497 (0.74), 7.597 (1.22), 7.616 (0.96), 7.637 (2.00), 8.195 (1.21), 8.211 (1.20), 8.361 (1.23), 8.381 (1.18), 8.630 (4.29).

### EXAMPLE 26

### N-{(3S)-1-[2-methyl-4-({(1R)-1-[2-methyl-3-(trifluoromethyl)phenyl]ethyl}amino)pyrido[3,4-d]pyrimidin-6-yl]pyrrolidin-3-yl}acetamide

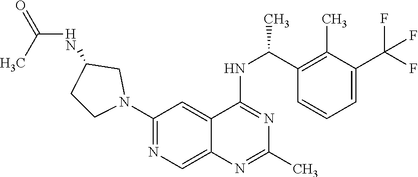 [SMILES: CC(=O)N[C@H]1CCN(c2cc3c(N[C@H](C)c4cccc(C(F)(F)F)c4C)nc(C)nc3cn2)C1]

Using the method described for Example 25: Intermediate 10 was treated with (1R)-1-[2-methyl-3-(trifluoromethyl)phenyl]ethan-1-amine hydrochloride (50.0 mg, 209 μmol) and gave the titled compound (30 mg, 35%) after preparative HPLC purification (basic method).

¹H-NMR (400 MHz, DMSO-d6) δ [ppm]: 1.557 (4.32), 1.575 (4.42), 1.831 (16.00), 1.928 (0.56), 1.941 (0.56), 1.960 (0.41), 2.178 (0.45), 2.194 (0.54), 2.209 (0.49), 2.226 (0.40), 2.281 (13.18), 2.518 (3.92), 2.523 (2.45), 2.539 (0.42), 2.618 (5.45), 3.313 (0.80), 3.349 (1.00), 3.554 (0.55), 3.567 (0.64), 3.574 (0.62), 3.585 (0.71), 3.602 (0.99), 3.620 (0.51), 3.628 (0.49), 3.643 (0.82), 3.658 (0.90), 3.670 (0.76), 3.685 (0.68), 4.402 (0.56), 4.414 (0.56), 5.682 (0.65), 5.699 (1.02), 5.716 (0.66), 7.062 (2.74), 7.338 (0.63), 7.357 (1.37), 7.377 (0.80), 7.527 (1.47), 7.546 (1.20), 7.737 (1.29), 7.756 (1.17), 8.202 (1.19), 8.218 (1.14), 8.492 (1.11), 8.510 (1.07), 8.613 (4.06).

TABLE 1
Examples 27-34
Using the method described for Example 25: Intermediate 7 was treated with the corresponding
phenylethan-1-amines or their hydrochloride salts and gave the desired compounds after preparative
HPLC purification (basic method).

| | Structure |
|---|---|
| | IUPAC-Name |
| Example | ¹H-NMR |
| 27 | 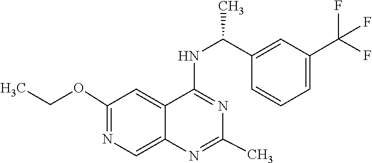 [SMILES: CCOc1cc2c(N[C@H](C)c3cccc(C(F)(F)F)c3)nc(C)nc2cn1] |
| | 6-ethoxy-2-methyl-N-{(1R)-1-[3-(trifluoromethyl)phenyl]ethyl}pyrido[3,4-d]pyrimidin-4- |
| | amine |
| | ¹H-NMR (400 MHz, DMSO-d6) δ [ppm]: 1.344 (4.11), 1.361 (9.28), 1.379 (4.35), 1.596 |
| | (5.13), 1.613 (5.14), 2.373 (16.00), 2.518 (1.10), 2.522 (0.78), 4.332 (1.19), 4.350 (4.19), |
| | 4.368 (4.02), 4.385 (1.14), 5.594 (0.70), 5.613 (1.05), 5.630 (0.69), 7.541 (0.44), 7.560 |
| | (1.39), 7.578 (1.87), 7.586 (1.89), 7.606 (0.50), 7.694 (3.62), 7.696 (3.60), 7.739 (1.23), |
| | 7.756 (0.95), 7.818 (1.96), 8.535 (1.15), 8.554 (1.11), 8.694 (4.06). |
| 28 | 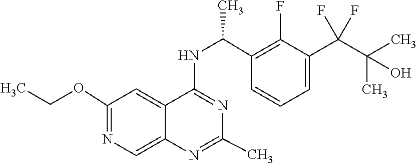 [SMILES: CCOc1cc2c(N[C@H](C)c3cccc(C(F)(F)C(C)(C)O)c3F)nc(C)nc2cn1] |
| | 1-(3-{(1R)-1-[(6-ethoxy-2-methylpyrido[3,4-d]pyrimidin-4-yl)amino]ethyl}-2- |
| | fluorophenyl)-1,1-difluoro-2-methylpropan-2-ol |
| | ¹H-NMR (400 MHz, DMSO-d6) δ [ppm]: 1.200 (5.81), 1.226 (5.85), 1.351 (4.38), 1.369 |
| | (10.21), 1.387 (4.40), 1.563 (4.50), 1.580 (4.47), 2.328 (16.00), 2.518 (2.01), 2.523 (1.46), |
| | 2.669 (0.49), 4.338 (1.21), 4.356 (4.12), 4.373 (4.00), 4.391 (1.16), 5.331 (4.46), 5.739 |
| | (0.66), 5.756 (1.02), 5.775 (0.66), 7.188 (0.66), 7.208 (1.59), 7.227 (1.03), 7.289 (0.59), |
| | 7.293 (0.73), 7.310 (1.00), 7.326 (0.48), 7.330 (0.45), 7.569 (0.53), 7.585 (0.94), 7.601 |
| | (0.49), 7.752 (3.52), 7.754 (3.50), 8.556 (1.09), 8.575 (1.05), 8.693 (4.12). |
| 29 | 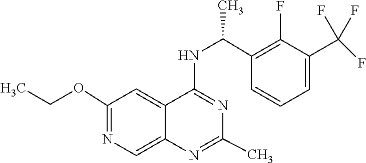 [SMILES: CCOc1cc2c(N[C@H](C)c3cccc(C(F)(F)F)c3F)nc(C)nc2cn1] |
| | 6-ethoxy-N-{(1R)-1-[2-fluoro-3-(trifluoromethyl)phenyl]ethyl}-2-methylpyrido[3,4- |
| | d]pyrimidin-4-amine |
| | ¹H-NMR (400 MHz, DMSO-d6) δ [ppm]: 1.349 (4.06), 1.366 (9.86), 1.384 (4.46), 1.603 |
| | (4.95), 1.621 (4.95), 2.324 (16.00), 2.518 (3.01), 2.523 (2.20), 2.669 (0.40), 4.338 (1.20), |
| | 4.355 (4.11), 4.373 (4.23), 4.391 (1.22), 5.704 (0.67), 5.722 (1.03), 5.739 (0.68), 7.333 |
| | (0.68), 7.353 (1.50), 7.372 (0.85), 7.629 (0.62), 7.646 (1.08), 7.665 (0.54), 7.733 (3.61), |
| | 7.735 (3.58), 7.776 (0.59), 7.794 (1.06), 7.811 (0.56), 8.609 (0.97), 8.627 (0.96), 8.698 |
| | (3.94), 8.700 (3.97). |
| 30 | 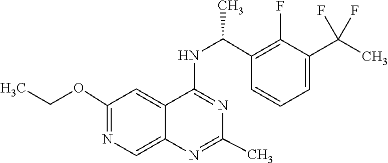 [SMILES: CCOc1cc2c(N[C@H](C)c3cccc(C(C)(F)F)c3F)nc(C)nc2cn1] |
| | N-{(1R)-1-[3-(1,1-difluoroethyl)-2-fluorophenyl]ethyl}-6-ethoxy-2-methylpyrido[3,4- |
| | d]pyrimidin-4-amine |
| | ¹H-NMR (400 MHz, DMSO-d6) δ [ppm]: 1.349 (4.33), 1.367 (9.85), 1.384 (4.37), 1.582 |
| | (5.06), 1.600 (5.06), 1.978 (2.66), 2.026 (5.16), 2.074 (2.43), 2.322 (0.58), 2.327 (0.87), |
| | 2.341 (16.00), 2.518 (5.41), 2.522 (3.88), 2.664 (0.51), 2.669 (0.70), 2.673 (0.50), 4.337 |
| | (1.22), 4.355 (4.19), 4.372 (4.26), 4.390 (1.22), 5.734 (0.76), 5.752 (1.16), 5.770 (0.76), |
| | 7.224 (0.81), 7.243 (1.82), 7.262 (1.08), 7.415 (0.68), 7.433 (1.14), 7.450 (0.58), 7.603 |
| | (0.59), 7.620 (1.09), 7.637 (0.57), 7.743 (3.77), 7.745 (3.65), 8.565 (1.18), 8.583 (1.13), |
| | 8.695 (4.12). |
| 31 | 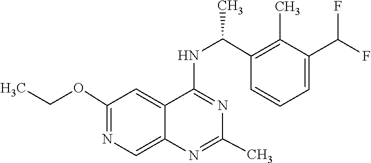 [SMILES: CCOc1cc2c(N[C@H](C)c3cccc(C(F)F)c3C)nc(C)nc2cn1] |
| | N-{(1R)-1-[3-(difluoromethyl)-2-methylphenyl]ethyl}-6-ethoxy-2-methylpyrido[3,4- |
| | d]pyrimidin-4-amine |
| | ¹H-NMR (400 MHz, DMSO-d6) δ [ppm]: 1.345 (4.29), 1.363 (10.36), 1.380 (4.65), 1.525 |
| | (4.96), 1.542 (4.97), 2.327 (0.46), 2.353 (16.00), 2.518 (1.61), 2.523 (1.16), 2.545 (7.41), |
| | 4.328 (1.23), 4.345 (4.35), 4.362 (4.30), 4.380 (1.17), 5.703 (0.72), 5.721 (1.11), 5.739 |
| | (0.71), 7.073 (0.94), 7.211 (1.99), 7.267 (0.68), 7.287 (1.61), 7.305 (1.06), 7.348 (0.83), |
| | 7.376 (1.54), 7.394 (1.03), 7.637 (1.24), 7.656 (1.10), 7.738 (3.73), 7.740 (3.75), 8.606 |
| | (1.14), 8.624 (1.13), 8.671 (4.24), 8.673 (4.11). |
| 32 | 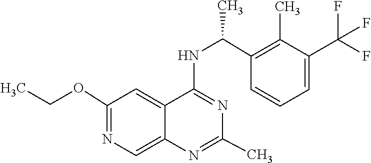 [SMILES: CCOc1cc2c(N[C@H](C)c3cccc(C(F)(F)F)c3C)nc(C)nc2cn1] |
| | 6-ethoxy-2-methyl-N-{(1R)-1-[2-methyl-3-(trifluoromethyl)phenyl]ethyl}pyrido[3,4- |
| | d]pyrimidin-4-amine |
| | ¹H-NMR (400 MHz, DMSO-d6) δ [ppm]: 1.199 (5.62), 1.215 (5.65), 1.231 (0.55), 1.237 |
| | (0.50), 1.339 (4.30), 1.357 (9.83), 1.375 (4.33), 2.518 (2.00), 2.522 (1.37), 2.587 (16.00), |
| | 4.271 (1.15), 4.288 (1.16), 4.346 (1.20), 4.363 (3.81), 4.381 (3.83), 4.398 (1.14), 5.166 |
| | (0.72), 5.175 (0.66), 5.199 (1.36), 5.207 (1.36), 5.232 (0.68), 5.238 (0.76), 6.958 (3.91), |
| | 6.959 (4.07), 7.292 (0.77), 7.311 (1.71), 7.331 (0.98), 7.508 (0.64), 7.526 (1.04), 7.541 |
| | (0.59), 7.545 (0.55), 7.755 (0.54), 7.773 (0.97), 7.788 (0.51), 8.977 (3.44), 8.979 (3.57). |
| 33 | 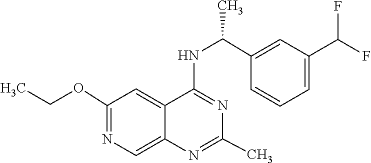 [SMILES: CCOc1cc2c(N[C@H](C)c3cccc(C(F)F)c3)nc(C)nc2cn1] |
| | N-{(1R)-1-[3-(difluoromethyl)phenyl]ethyl}-6-ethoxy-2-methylpyrido[3,4-d]pyrimidin-4- |
| | amine |
| | ¹H-NMR (400 MHz, DMSO-d6) δ [ppm]: 1.344 (4.15), 1.361 (9.55), 1.379 (4.24), 1.584 |
| | (5.30), 1.602 (5.33), 2.377 (16.00), 2.522 (1.20), 4.330 (1.20), 4.348 (4.17), 4.365 (4.08), |
| | 4.382 (1.18), 5.595 (0.68), 5.613 (1.03), 5.631 (0.68), 6.881 (1.25), 7.021 (2.56), 7.161 |
| | (1.17), 7.415 (0.72), 7.434 (1.62), 7.454 (1.25), 7.473 (1.77), 7.492 (0.71), 7.604 (1.20), |
| | 7.622 (0.97), 7.652 (2.02), 7.711 (3.77), 8.524 (1.11), 8.544 (1.10), 8.690 (4.13). |
| 34 | 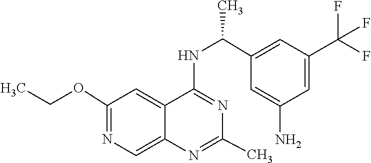 [SMILES: CCOc1cc2c(N[C@H](C)c3cc(N)cc(C(F)(F)F)c3)nc(C)nc2cn1] |
| | N-{(1R)-1-[3-amino-5-(trifluoromethyl)phenyl]ethyl}-6-ethoxy-2-methylpyrido[3,4- |
| | d]pyrimidin-4-amine |
| | ¹H-NMR (400 MHz, DMSO-d6) δ [ppm]: 1.233 (0.46), 1.344 (4.34), 1.361 (9.40), 1.379 |
| | (4.76), 1.529 (6.60), 1.547 (6.94), 1.629 (0.45), 2.327 (0.76), 2.391 (16.00), 2.669 (0.80), |
| | 4.329 (1.40), 4.346 (4.34), 4.364 (4.42), 4.382 (1.51), 5.484 (1.12), 5.502 (1.71), 5.520 |
| | (1.25), 5.558 (6.09), 6.694 (3.72), 6.834 (3.63), 6.884 (3.88), 7.715 (5.27), 8.450 (1.88), |
| | 8.470 (1.93), 8.694 (5.20). |

TABLE 2
Examples 35-42
Using the method described for Example 25: Intermediate 8 was treated with the corresponding
phenylethan-1-amines or their hydrochloride salts and gave the desired compounds after preparative
HPLC purification (basic method).

| | Structure |
|---|---|
| | IUPAC-Name |
| Example | ¹H-NMR |
| 35 | 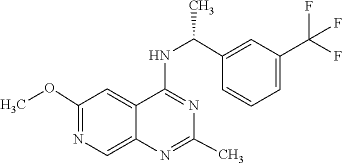 [SMILES: COc1cc2c(N[C@H](C)c3cccc(C(F)(F)F)c3)nc(C)nc2cn1] |
| | 6-methoxy-2-methyl-N-{(1R)-1-[3-(trifluoromethyl)phenyl]ethyl}pyrido[3,4-d]pyrimidin- |
| | 4-amine |
| | ¹H-NMR (400 MHz, DMSO-d6) δ [ppm]: 1.605 (4.33), 1.622 (4.35), 2.376 (13.30), 2.518 |
| | (0.84), 2.523 (0.56), 3.952 (16.00), 5.604 (0.57), 5.621 (0.87), 5.640 (0.57), 7.563 (1.13), |
| | 7.581 (1.59), 7.587 (1.55), 7.608 (0.40), 7.702 (3.00), 7.704 (3.01), 7.741 (0.99), 7.759 |
| | (0.76), 7.819 (1.58), 8.584 (0.92), 8.603 (0.91), 8.713 (3.17). |
| 36 | 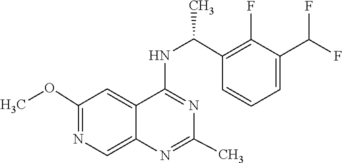 [SMILES: COc1cc2c(N[C@H](C)c3cccc(C(F)F)c3F)nc(C)nc2cn1] |
| | N-{(1R)-1-[3-(difluoromethyl)-2-fluorophenyl]ethyl}-6-methoxy-2-methylpyrido[3,4- |
| | d]pyrimidin-4-amine |
| | ¹H-NMR (400 MHz, DMSO-d6) δ [ppm]: 1.595 (4.25), 1.612 (4.26), 2.345 (13.57), 2.518 |
| | (1.43), 2.523 (0.99), 2.539 (0.53), 3.958 (16.00), 5.748 (0.63), 5.765 (0.98), 5.783 (0.63), |
| | 7.100 (0.92), 7.236 (1.94), 7.270 (0.67), 7.289 (1.47), 7.308 (0.86), 7.372 (0.83), 7.486 |
| | (0.51), 7.504 (0.87), 7.521 (0.43), 7.649 (0.47), 7.667 (0.87), 7.685 (0.44), 7.751 (3.10), |
| | 7.753 (3.19), 8.611 (1.00), 8.630 (0.96), 8.717 (3.46). |
| 37 | 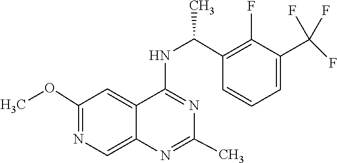 [SMILES: COc1cc2c(N[C@H](C)c3cccc(C(F)(F)F)c3F)nc(C)nc2cn1] |
| | N-{(1R)-1-[2-fluoro-3-(trifluoromethyl)phenyl]ethyl}-6-methoxy-2-methylpyrido[3,4- |
| | d]pyrimidin-4-amine |
| | ¹H-NMR (400 MHz, DMSO-d6) δ [ppm]: 1.612 (4.13), 1.629 (4.10), 2.327 (13.80), 2.427 |
| | (0.53), 2.518 (1.84), 2.523 (1.29), 2.669 (0.44), 3.931 (0.62), 3.959 (16.00), 5.708 (0.61), |
| | 5.726 (0.96), 5.744 (0.61), 7.335 (0.56), 7.354 (1.20), 7.374 (0.67), 7.630 (0.49), 7.647 |
| | (0.85), 7.664 (0.41), 7.741 (3.08), 7.743 (3.02), 7.777 (0.46), 7.794 (0.83), 7.811 (0.42), |
| | 8.655 (0.91), 8.672 (0.88), 8.718 (3.22). |
| 38 | 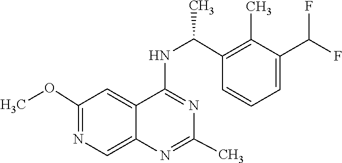 [SMILES: COc1cc2c(N[C@H](C)c3cccc(C(F)F)c3C)nc(C)nc2cn1] |
| | N-{(1R)-1-[3-(difluoromethyl)-2-methylphenyl]ethyl}-6-methoxy-2-methylpyrido[3,4- |
| | d]pyrimidin-4-amine |
| | ¹H-NMR (400 MHz, DMSO-d6) δ [ppm]: 1.532 (4.13), 1.550 (4.16), 2.356 (13.73), 2.428 |
| | (0.91), 2.518 (1.81), 2.523 (1.32), 2.544 (6.10), 3.931 (1.07), 3.950 (16.00), 5.708 (0.60), |
| | 5.726 (0.92), 5.744 (0.60), 7.074 (0.78), 7.212 (1.63), 7.270 (0.57), 7.289 (1.32), 7.308 |
| | (0.88), 7.349 (0.72), 7.377 (1.27), 7.395 (0.85), 7.638 (1.01), 7.657 (0.91), 7.746 (3.12), |
| | 7.747 (3.10), 8.651 (0.96), 8.670 (0.93), 8.691 (3.40), 8.693 (3.39). |
| 39 | 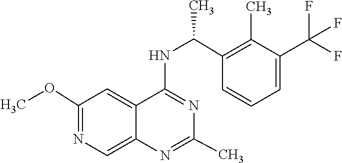 [SMILES: COc1cc2c(N[C@H](C)c3cccc(C(F)(F)F)c3C)nc(C)nc2cn1] |
| | 6-methoxy-2-methyl-N-{(1R)-1-[2-methyl-3-(trifluoromethyl)phenyl]ethyl}pyrido[3,4- |
| | d]pyrimidin-4-amine |
| | ¹H-NMR (400 MHz, DMSO-d6) δ [ppm]: 1.549 (4.21), 1.567 (4.16), 2.327 (0.70), 2.335 |
| | (14.29), 2.427 (0.63), 2.518 (1.53), 2.523 (1.07), 2.618 (4.65), 3.930 (0.70), 3.953 (16.00), |
| | 5.675 (0.60), 5.693 (0.96), 5.711 (0.60), 7.332 (0.52), 7.351 (1.16), 7.371 (0.68), 7.530 |
| | (1.25), 7.548 (1.00), 7.742 (3.17), 7.744 (3.24), 7.749 (1.28), 7.769 (1.01), 8.696 (3.53), |
| | 8.705 (0.98), 8.723 (0.92). |
| 40 | 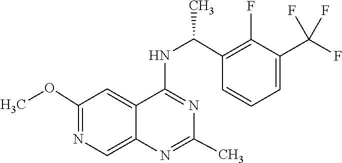 [SMILES: COc1cc2c(N[C@H](C)c3cccc(C(F)(F)F)c3F)nc(C)nc2cn1] |
| | N-{(1R)-1-[3-(1,1-difluoroethyl)-2-fluorophenyl]ethyl}-6-methoxy-2-methylpyrido[3,4- |
| | d]pyrimidin-4-amine |
| | ¹H-NMR (400 MHz, DMSO-d6) δ [ppm]: 1.590 (4.71), 1.608 (4.65), 1.979 (2.44), 2.027 |
| | (4.68), 2.075 (2.17), 2.251 (1.03), 2.323 (0.41), 2.327 (0.60), 2.332 (0.57), 2.344 (14.21), |
| | 2.518 (2.78), 2.523 (1.78), 2.669 (0.50), 3.881 (1.27), 3.958 (16.00), 5.739 (0.65), 5.757 |
| | (1.01), 5.775 (0.64), 7.225 (0.75), 7.244 (1.67), 7.264 (0.97), 7.416 (0.63), 7.435 (1.04), |
| | 7.450 (0.50), 7.604 (0.56), 7.622 (1.00), 7.639 (0.51), 7.751 (3.41), 8.613 (0.97), 8.630 |
| | (0.94), 8.714 (3.70). |
| 41 | 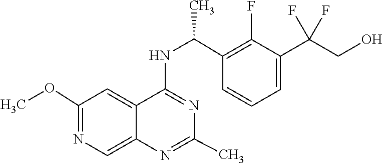 [SMILES: COc1cc2c(N[C@H](C)c3cccc(C(F)(F)CO)c3F)nc(C)nc2cn1] |
| | 2,2-difluoro-2-(2-fluoro-3-{(1R)-1-[(6-methoxy-2-methylpyrido[3,4-d]pyrimidin-4- |
| | yl)amino]ethyl}phenyl)ethan-1-ol |
| | ¹H-NMR (400 MHz, DMSO-d6) δ [ppm]: 1.584 (4.72), 1.602 (4.78), 2.352 (14.39), 3.896 |
| | (0.94), 3.932 (1.86), 3.958 (16.00), 5.728 (0.86), 5.750 (0.83), 5.767 (1.08), 5.785 (0.70), |
| | 7.231 (0.78), 7.251 (1.79), 7.270 (1.08), 7.400 (0.69), 7.417 (1.12), 7.433 (0.58), 7.617 |
| | (0.59), 7.634 (1.09), 7.651 (0.57), 7.751 (3.58), 8.605 (0.98), 8.623 (0.98), 8.715 (3.90). |
| 42 | 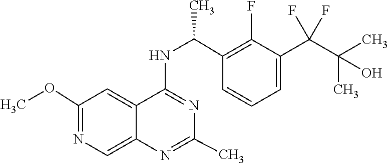 [SMILES: COc1cc2c(N[C@H](C)c3cccc(C(F)(F)C(C)(C)O)c3F)nc(C)nc2cn1] |
| | 1,1-difluoro-1-(2-fluoro-3-{(1R)-1-[(6-methoxy-2-methylpyrido[3,4-d]pyrimidin-4- |
| | yl)amino]ethyl}phenyl)-2-methylpropan-2-ol |
| | ¹H-NMR (400 MHz, DMSO-d6) δ [ppm]: 1.200 (5.51), 1.225 (5.69), 1.570 (4.17), 1.588 |
| | (4.23), 2.331 (14.88), 2.518 (5.25), 2.523 (3.82), 2.665 (0.52), 2.669 (0.74), 2.673 (0.53), |
| | 3.959 (16.00), 5.333 (5.89), 5.744 (0.64), 5.762 (0.98), 5.779 (0.66), 7.191 (0.61), 7.210 |
| | (1.48), 7.229 (0.98), 7.294 (0.66), 7.312 (0.98), 7.327 (0.50), 7.569 (0.50), 7.586 (0.91), |
| | 7.602 (0.48), 7.760 (3.35), 7.762 (3.32), 8.604 (1.08), 8.623 (1.07), 8.713 (3.71). |

TABLE 3
Examples 43-91
Using the method described for Example 25: Intermediate 9 was treated with the corresponding
phenylethan-1-amines or their hydrochloride salts and gave the desired compounds after preparative
HPLC purification (basic method).

| | Structure |
|---|---|
| | IUPAC-Name |
| Example | ¹H-NMR |
| 43 | 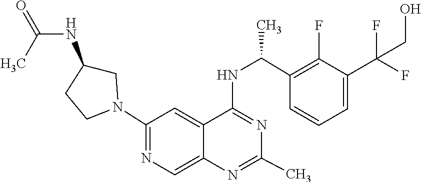 [SMILES: CC(=O)N[C@@H]1CCN(c2cc3c(N[C@H](C)c4cccc(C(F)(F)CO)c4F)nc(C)nc3cn2)C1] |
| | N-{(3R)-1-[4-({(1R)-1-[3-(1,1-difluoro-2-hydroxyethyl)-2-fluorophenyl]ethyl}amino)-2- |
| | methylpyrido[3,4-d]pyrimidin-6-yl]pyrrolidin-3-yl}acetamide |
| | ¹H-NMR (400 MHz, DMSO-d6) δ [ppm]: 1.592 (1.80), 1.610 (1.79), 1.827 (6.60), 2.296 |
| | (5.77), 2.518 (0.86), 2.523 (0.55), 2.539 (16.00), 3.313 (0.43), 3.942 (0.42), 5.704 (0.46), |
| | 5.721 (1.11), 5.737 (0.44), 5.782 (0.44), 7.078 (1.15), 7.256 (0.73), 7.275 (0.43), 7.413 |
| | (0.42), 7.613 (0.41), 8.196 (0.51), 8.213 (0.50), 8.385 (0.48), 8.404 (0.46), 8.631 (1.73). |
| 44 | 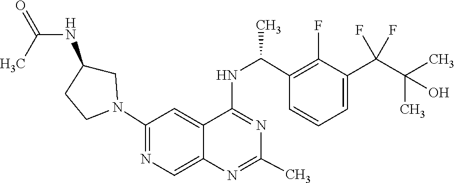 [SMILES: CC(=O)N[C@@H]1CCN(c2cc3c(N[C@H](C)c4cccc(C(F)(F)C(C)(C)O)c4F)nc(C)nc3cn2)C1] |
| | N-{(3R)-1-[4-({(1R)-1-[3-(1,1-difluoro-2-hydroxy-2-methylpropyl)-2- |
| | fluorophenyl]ethyl}amino)-2-methylpyrido[3,4-d]pyrimidin-6-yl]pyrrolidin-3- |
| | yl}acetamide |
| | ¹H-NMR (400 MHz, DMSO-d6) δ [ppm]: 1.107 (16.00), 1.203 (4.00), 1.231 (4.31), 1.578 |
| | (2.79), 1.595 (2.81), 1.828 (8.98), 1.947 (0.41), 2.276 (8.04), 3.303 (0.52), 3.314 (0.64), |
| | 3.623 (0.57), 3.648 (0.43), 3.668 (0.61), 3.683 (0.59), 3.695 (0.51), 3.710 (0.45), 4.193 |
| | (1.28), 4.400 (0.42), 4.411 (0.43), 5.338 (2.96), 5.761 (0.44), 5.779 (0.68), 5.796 (0.44), |
| | 7.087 (1.84), 7.195 (0.42), 7.214 (1.04), 7.234 (0.69), 7.291 (0.49), 7.308 (0.71), 7.565 |
| | (0.68), 8.201 (0.82), 8.218 (0.82), 8.391 (0.80), 8.410 (0.77), 8.629 (2.85). |
| 45 | 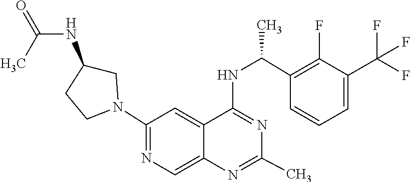 [SMILES: CC(=O)N[C@@H]1CCN(c2cc3c(N[C@H](C)c4cccc(C(F)(F)F)c4F)nc(C)nc3cn2)C1] |
| | N-{(3R)-1-[4-({(1R)-1-[2-fluoro-3-(trifluoromethyl)phenyl]ethyl}amino)-2- |
| | methylpyrido[3,4-d]pyrimidin-6-yl]pyrrolidin-3-yl}acetamide |
| | ¹H-NMR (400 MHz, DMSO-d6) δ [ppm]: 1.107 (1.40), 1.230 (0.55), 1.620 (4.74), 1.638 |
| | (4.75), 1.827 (16.00), 1.933 (0.57), 1.947 (0.60), 1.965 (0.43), 2.184 (0.47), 2.200 (0.57), |
| | 2.216 (0.50), 2.271 (14.13), 2.332 (0.55), 2.518 (2.51), 2.522 (1.65), 2.673 (0.56), 3.301 |
| | (0.91), 3.312 (1.31), 3.526 (0.46), 3.532 (0.56), 3.539 (0.45), 3.545 (0.61), 3.551 (0.55), |
| | 3.565 (0.44), 3.620 (0.89), 3.639 (0.49), 3.646 (0.61), 3.668 (0.86), 3.684 (0.94), 3.694 |
| | (0.80), 3.710 (0.72), 4.395 (0.62), 4.408 (0.60), 5.720 (0.73), 5.737 (1.13), 5.755 (0.74), |
| | 5.758 (0.68), 7.072 (2.94), 7.340 (0.71), 7.359 (1.51), 7.379 (0.84), 7.625 (0.63), 7.642 |
| | (1.09), 7.660 (0.53), 7.753 (0.59), 7.770 (1.05), 7.788 (0.52), 8.198 (1.19), 8.215 (1.17), |
| | 8.444 (1.18), 8.461 (1.12), 8.632 (4.33). |
| 46 | 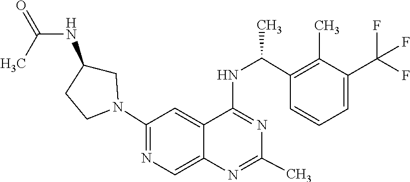 [SMILES: CC(=O)N[C@@H]1CCN(c2cc3c(N[C@H](C)c4cccc(C(F)(F)F)c4C)nc(C)nc3cn2)C1] |
| | N-{(3R)-1-[2-methyl-4-({(1R)-1-[2-methyl-3- |
| | (trifluoromethyl)phenyl]ethyl}amino)pyrido[3,4-d]pyrimidin-6-yl]pyrrolidin-3- |
| | yl}acetamide |
| | ¹H-NMR (400 MHz, DMSO-d6) δ [ppm]: −0.002 (0.47), 1.556 (4.44), 1.574 (4.53), 1.806 |
| | (0.45), 1.827 (16.00), 1.896 (0.67), 1.931 (0.54), 1.945 (0.59), 1.963 (0.41), 2.181 (0.44), |
| | 2.199 (0.57), 2.214 (0.51), 2.283 (13.52), 2.336 (0.69), 2.367 (0.54), 2.518 (9.15), 2.523 |
| | (6.04), 2.539 (0.70), 2.618 (5.79), 2.678 (0.68), 3.302 (0.88), 3.527 (0.54), 3.539 (0.59), |
| | 3.547 (0.52), 3.616 (0.87), 3.635 (0.49), 3.642 (0.60), 3.663 (0.84), 3.679 (0.90), 3.691 |
| | (0.76), 3.706 (0.68), 4.394 (0.58), 4.406 (0.56), 5.687 (0.68), 5.704 (1.06), 5.722 (0.69), |
| | 7.069 (2.82), 7.339 (0.67), 7.359 (1.40), 7.378 (0.83), 7.528 (1.57), 7.547 (1.26), 7.739 |
| | (1.35), 7.758 (1.21), 8.193 (1.15), 8.210 (1.14), 8.492 (1.16), 8.510 (1.09), 8.611 (4.09). |
| 47 | 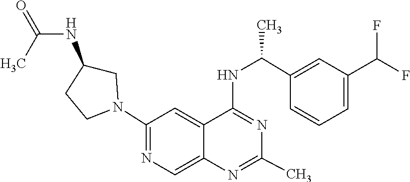 [SMILES: CC(=O)N[C@@H]1CCN(c2cc3c(N[C@H](C)c4cccc(C(F)F)c4)nc(C)nc3cn2)C1] |
| | N-{(3R)-1-[4-({(1R)-1-[3-(difluoromethyl)phenyl]ethyl}amino)-2-methylpyrido[3,4- |
| | d]pyrimidin-6-yl]pyrrolidin-3-yl}acetamide |
| | ¹H-NMR (400 MHz, DMSO-d6) δ [ppm]: 1.600 (4.66), 1.617 (4.67), 1.822 (16.00), 1.925 |
| | (0.54), 1.938 (0.56), 2.176 (0.44), 2.193 (0.53), 2.208 (0.47), 2.326 (14.43), 2.518 (2.87), |
| | 2.523 (1.96), 2.539 (1.38), 2.665 (0.58), 2.669 (0.83), 2.673 (0.59), 3.290 (0.65), 3.300 |
| | (0.72), 3.317 (1.11), 3.516 (0.43), 3.521 (0.52), 3.529 (0.41), 3.535 (0.57), 3.542 (0.51), |
| | 3.610 (0.85), 3.627 (0.46), 3.635 (0.59), 3.653 (0.95), 3.668 (0.89), 3.680 (0.77), 3.696 |
| | (0.69), 4.388 (0.58), 4.401 (0.57), 5.628 (0.63), 5.646 (0.94), 5.665 (0.62), 6.886 (1.20), |
| | 7.026 (2.47), 7.050 (2.79), 7.166 (1.08), 7.416 (0.69), 7.435 (1.43), 7.461 (1.07), 7.480 |
| | (1.62), 7.499 (0.69), 7.597 (1.13), 7.617 (0.88), 7.638 (1.87), 8.189 (1.12), 8.206 (1.10), |
| | 8.362 (1.16), 8.382 (1.12), 8.630 (4.07). |
| 48 | 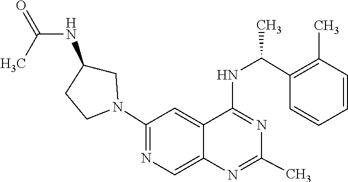 [SMILES: CC(=O)N[C@@H]1CCN(c2cc3c(N[C@H](C)c4ccccc4C)nc(C)nc3cn2)C1] |
| | N-[(3R)-1-(2-methyl-4-{[(1R)-1-(2-methylphenyl)ethyl]amino}pyrido[3,4-d]pyrimidin-6- |
| | yl)pyrrolidin-3-yl]acetamide |
| | ¹H-NMR (400 MHz, DMSO-d6) δ [ppm]: 1.107 (6.40), 1.521 (4.70), 1.539 (4.67), 1.825 |
| | (16.00), 1.926 (0.54), 1.939 (0.57), 2.176 (0.44), 2.193 (0.53), 2.207 (0.48), 2.315 (14.00), |
| | 2.327 (0.67), 2.332 (0.43), 2.478 (10.85), 2.518 (1.32), 2.523 (0.88), 3.299 (0.72), 3.309 |
| | (0.86), 3.515 (0.43), 3.521 (0.53), 3.528 (0.42), 3.533 (0.57), 3.540 (0.51), 3.610 (0.87), |
| | 3.629 (0.47), 3.636 (0.61), 3.654 (0.97), 3.670 (0.91), 3.680 (0.78), 3.696 (0.70), 4.391 |
| | (0.57), 4.405 (0.56), 5.653 (0.67), 5.671 (1.02), 5.689 (0.66), 7.074 (3.13), 7.090 (1.08), |
| | 7.106 (1.51), 7.109 (1.44), 7.125 (2.07), 7.133 (1.09), 7.139 (1.03), 7.152 (1.12), 7.169 |
| | (0.47), 7.173 (0.42), 7.451 (1.42), 7.470 (1.15), 8.191 (1.12), 8.208 (1.11), 8.373 (1.14), |
| | 8.392 (1.09), 8.602 (4.13). |
| 49 | 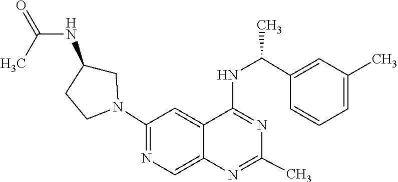 [SMILES: CC(=O)N[C@@H]1CCN(c2cc3c(N[C@H](C)c4cccc(C)c4)nc(C)nc3cn2)C1] |
| | N-[(3R)-1-(2-methyl-4-{[(1R)-1-(3-methylphenyl)ethyl]amino}pyrido[3,4-d]pyrimidin-6- |
| | yl)pyrrolidin-3-yl]acetamide |
| | ¹H-NMR (400 MHz, DMSO-d6) δ [ppm]: 1.108 (16.00), 1.557 (5.21), 1.575 (5.24), 1.821 |
| | (15.34), 1.919 (0.60), 1.933 (0.62), 1.950 (0.45), 2.171 (0.50), 2.187 (0.60), 2.204 (0.55), |
| | 2.219 (0.42), 2.286 (12.33), 2.337 (13.90), 2.523 (1.90), 2.540 (8.10), 2.669 (0.46), 3.287 |
| | (0.90), 3.297 (1.05), 3.511 (0.51), 3.517 (0.60), 3.530 (0.65), 3.537 (0.59), 3.549 (0.43), |
| | 3.588 (0.43), 3.606 (0.95), 3.624 (0.55), 3.631 (0.65), 3.647 (0.98), 3.662 (0.98), 3.674 |
| | (0.83), 3.689 (0.75), 4.194 (0.45), 4.387 (0.65), 4.400 (0.64), 5.586 (0.69), 5.605 (1.00), |
| | 5.623 (0.70), 7.027 (0.89), 7.031 (0.89), 7.041 (1.07), 7.056 (3.12), 7.184 (0.41), 7.203 |
| | (2.17), 7.215 (3.39), 7.218 (3.45), 7.244 (2.19), 8.190 (1.19), 8.207 (1.20), 8.275 (1.23), |
| | 8.295 (1.20), 8.621 (4.38). |
| 50 | 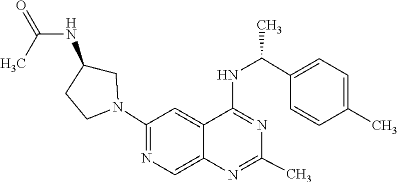 [SMILES: CC(=O)N[C@@H]1CCN(c2cc3c(N[C@H](C)c4ccc(C)cc4)nc(C)nc3cn2)C1] |
| | N-[(3R)-1-(2-methyl-4-{[(1R)-1-(4-methylphenyl)ethyl]amino}pyrido[3,4-d]pyrimidin-6- |
| | yl)pyrrolidin-3-yl]acetamide |
| | ¹H-NMR (400 MHz, DMSO-d6) δ [ppm]: 1.107 (0.43), 1.231 (0.49), 1.550 (5.76), 1.568 |
| | (5.84), 1.819 (16.00), 1.828 (1.76), 1.904 (0.46), 1.918 (0.77), 1.931 (0.83), 1.949 (0.67), |
| | 1.963 (0.47), 2.169 (0.67), 2.186 (0.78), 2.201 (0.71), 2.218 (0.58), 2.254 (11.04), 2.278 |
| | (1.11), 2.329 (14.60), 2.669 (0.63), 3.285 (0.85), 3.295 (0.92), 3.311 (1.06), 3.515 (0.71), |
| | 3.528 (0.79), 3.548 (0.55), 3.582 (0.48), 3.601 (1.09), 3.627 (0.85), 3.641 (1.11), 3.657 |
| | (1.13), 3.668 (0.99), 3.683 (0.89), 3.855 (0.76), 4.384 (0.83), 4.397 (0.85), 4.411 (0.53), |
| | 5.570 (0.76), 5.588 (1.15), 5.607 (0.79), 7.049 (3.56), 7.113 (3.21), 7.132 (3.78), 7.296 |
| | (4.49), 7.316 (3.82), 8.185 (1.45), 8.202 (1.57), 8.254 (1.47), 8.274 (1.46), 8.617 (5.01). |
| 51 | 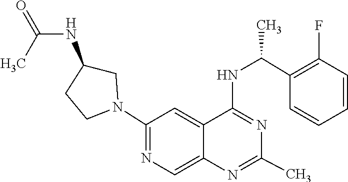 [SMILES: CC(=O)N[C@@H]1CCN(c2cc3c(N[C@H](C)c4ccccc4F)nc(C)nc3cn2)C1] |
| | N-[(3R)-1-(4-{[(1R)-1-(2-fluorophenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.576 (4.92), 1.594 (4.87), 1.827 (16.00), 1.931 |
| | (0.56), 1.945 (0.59), 1.962 (0.42), 2.182 (0.47), 2.198 (0.57), 2.213 (0.50), 2.298 (14.21), |
| | 2.518 (0.53), 3.304 (1.05), 3.314 (1.34), 3.525 (0.47), 3.530 (0.57), 3.537 (0.46), 3.544 |
| | (0.61), 3.550 (0.55), 3.563 (0.41), 3.602 (0.40), 3.620 (0.88), 3.627 (0.43), 3.638 (0.50), |
| | 3.646 (0.61), 3.665 (0.99), 3.680 (0.94), 3.691 (0.80), 3.706 (0.72), 4.396 (0.60), 4.409 |
| | (0.59), 5.778 (0.68), 5.796 (1.03), 5.814 (0.67), 7.095 (2.90), 7.131 (0.66), 7.133 (0.80), |
| | 7.139 (0.74), 7.142 (0.79), 7.152 (1.72), 7.160 (1.08), 7.162 (1.21), 7.169 (1.75), 7.186 |
| | (1.05), 7.189 (0.81), 7.245 (0.47), 7.249 (0.52), 7.259 (0.55), 7.263 (0.86), 7.269 (0.71), |
| | 7.280 (0.61), 7.283 (0.69), 7.445 (0.64), 7.449 (0.64), 7.465 (1.18), 7.469 (1.11), 7.484 |
| | (0.59), 7.489 (0.54), 8.202 (1.13), 8.219 (1.12), 8.345 (1.08), 8.364 (1.04), 8.629 (4.30). |
| 52 | 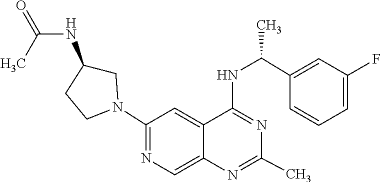 [SMILES: CC(=O)N[C@@H]1CCN(c2cc3c(N[C@H](C)c4cccc(F)c4)nc(C)nc3cn2)C1] |
| | N-[(3R)-1-(4-{[(1R)-1-(3-fluorophenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.577 (5.06), 1.594 (5.08), 1.822 (16.00), 1.925 |
| | (0.57), 1.938 (0.60), 1.955 (0.42), 2.176 (0.47), 2.193 (0.56), 2.208 (0.49), 2.332 (14.40), |
| | 2.518 (1.30), 2.523 (0.84), 3.294 (0.69), 3.304 (0.75), 3.321 (1.15), 3.517 (0.45), 3.523 |
| | (0.56), 3.530 (0.44), 3.536 (0.60), 3.542 (0.53), 3.556 (0.40), 3.593 (0.40), 3.611 (0.91), |
| | 3.629 (0.50), 3.637 (0.62), 3.655 (1.02), 3.670 (0.94), 3.682 (0.81), 3.698 (0.74), 4.389 |
| | (0.61), 4.402 (0.60), 5.608 (0.67), 5.626 (0.98), 5.644 (0.66), 7.026 (0.47), 7.031 (0.54), |
| | 7.033 (0.55), 7.046 (3.77), 7.069 (0.51), 7.074 (0.57), 7.226 (0.91), 7.231 (0.76), 7.256 |
| | (2.13), 7.275 (1.59), 7.342 (0.92), 7.358 (0.90), 7.362 (1.27), 7.377 (1.04), 7.381 (0.68), |
| | 7.397 (0.43), 8.188 (1.17), 8.205 (1.15), 8.304 (1.21), 8.323 (1.18), 8.635 (4.30). |
| 53 | 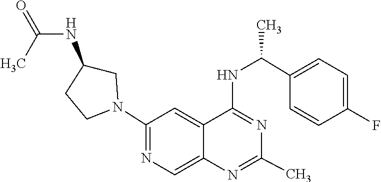 [SMILES: CC(=O)N[C@@H]1CCN(c2cc3c(N[C@H](C)c4ccc(F)cc4)nc(C)nc3cn2)C1] |
| | N-[(3R)-1-(4-{[(1R)-1-(4-fluorophenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.107 (0.90), 1.569 (4.94), 1.587 (4.96), 1.820 |
| | (16.00), 1.921 (0.56), 1.934 (0.59), 1.952 (0.42), 2.172 (0.47), 2.189 (0.56), 2.204 (0.50), |
| | 2.323 (0.44), 2.335 (13.76), 2.518 (1.44), 2.523 (0.93), 3.287 (0.75), 3.297 (0.86), 3.314 |
| | (1.19), 3.511 (0.44), 3.517 (0.55), 3.525 (0.44), 3.530 (0.60), 3.537 (0.54), 3.550 (0.40), |
| | 3.603 (0.88), 3.610 (0.41), 3.621 (0.49), 3.629 (0.61), 3.644 (0.89), 3.659 (0.93), 3.670 |
| | (0.80), 3.686 (0.71), 4.384 (0.59), 4.397 (0.58), 5.591 (0.62), 5.609 (0.92), 5.628 (0.62), |
| | 7.037 (2.84), 7.124 (2.05), 7.129 (0.67), 7.141 (0.77), 7.147 (4.13), 7.152 (0.82), 7.164 |
| | (0.69), 7.169 (2.25), 7.444 (1.91), 7.449 (0.79), 7.458 (2.09), 7.466 (1.91), 7.475 (0.72), |
| | 7.480 (1.68), 8.189 (1.12), 8.205 (1.10), 8.293 (1.16), 8.312 (1.12), 8.625 (4.17). |
| 54 | 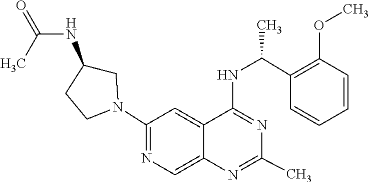 [SMILES: COc1ccccc1[C@@H](C)Nc1nc(C)nc2cnc(N3CC[C@@H](NC(C)=O)C3)cc12] |
| | N-[(3R)-1-(4-{[(1R)-1-(2-methoxyphenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.491 (5.05), 1.509 (5.24), 1.716 (0.40), 1.725 |
| | (0.62), 1.829 (15.88), 1.931 (0.57), 1.944 (0.60), 1.962 (0.42), 2.183 (0.48), 2.200 (0.57), |
| | 2.215 (0.52), 2.231 (0.61), 2.289 (13.84), 2.518 (0.61), 2.522 (0.90), 3.002 (0.43), 3.012 |
| | (0.42), 3.243 (0.42), 3.311 (1.40), 3.529 (0.48), 3.535 (0.59), 3.542 (0.46), 3.548 (0.64), |
| | 3.555 (0.59), 3.568 (0.41), 3.606 (0.41), 3.625 (0.90), 3.632 (0.43), 3.642 (0.50), 3.650 |
| | (0.63), 3.669 (0.99), 3.685 (0.93), 3.696 (0.80), 3.712 (0.71), 3.861 (16.00), 3.884 (0.51), |
| | 4.401 (0.59), 4.414 (0.58), 5.858 (0.67), 5.877 (0.91), 5.896 (0.66), 6.870 (0.78), 6.872 |
| | (0.82), 6.889 (1.63), 6.891 (1.63), 6.908 (0.93), 6.910 (0.93), 6.985 (1.45), 6.988 (1.50), |
| | 7.006 (1.89), 7.008 (1.71), 7.124 (2.82), 7.179 (0.96), 7.183 (0.96), 7.199 (1.13), 7.202 |
| | (1.18), 7.218 (0.65), 7.222 (0.66), 7.336 (1.39), 7.340 (1.32), 7.355 (1.28), 7.359 (1.16), |
| | 8.203 (1.24), 8.219 (1.96), 8.238 (1.14), 8.616 (4.20). |
| 55 | 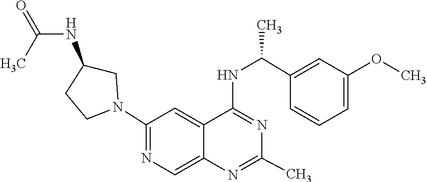 [SMILES: COc1cccc([C@@H](C)Nc2nc(C)nc3cnc(N4CC[C@@H](NC(C)=O)C4)cc23)c1] |
| | N-[(3R)-1-(4-{[(1R)-1-(3-methoxyphenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.563 (3.81), 1.580 (3.77), 1.710 (0.83), 1.718 |
| | (0.89), 1.727 (2.15), 1.735 (0.80), 1.744 (0.83), 1.821 (12.37), 1.921 (0.42), 1.934 (0.44), |
| | 2.190 (0.41), 2.323 (0.50), 2.328 (0.70), 2.337 (10.77), 2.518 (1.04), 2.523 (0.70), 2.987 |
| | (0.56), 2.997 (0.85), 3.003 (1.54), 3.007 (0.67), 3.013 (1.60), 3.020 (0.80), 3.030 (0.55), |
| | 3.290 (0.73), 3.301 (0.88), 3.519 (0.44), 3.532 (0.49), 3.539 (0.43), 3.604 (0.68), 3.630 |
| | (0.48), 3.647 (0.72), 3.662 (0.71), 3.674 (0.61), 3.689 (0.55), 3.726 (16.00), 4.388 (0.44), |
| | 4.401 (0.44), 5.585 (0.48), 5.604 (0.69), 5.622 (0.48), 6.780 (0.68), 6.784 (0.88), 6.789 |
| | (0.76), 6.801 (0.75), 6.803 (0.77), 6.807 (0.84), 6.989 (2.54), 6.993 (2.38), 7.010 (1.10), |
| | 7.053 (2.17), 7.219 (0.90), 7.240 (1.87), 7.260 (0.95), 8.192 (0.87), 8.208 (0.86), 8.271 |
| | (0.89), 8.292 (0.86), 8.625 (3.25). |
| 56 | 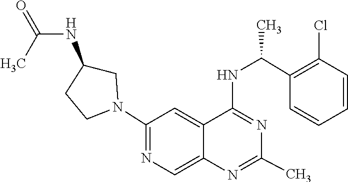 [SMILES: CC(=O)N[C@@H]1CCN(c2cc3c(N[C@H](C)c4ccccc4Cl)nc(C)nc3cn2)C1] |
| | N-[(3R)-1-(4-{[(1R)-1-(2-chlorophenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.107 (11.23), 1.551 (5.33), 1.569 (5.34), 1.820 |
| | (1.11), 1.829 (16.00), 1.936 (0.57), 1.949 (0.61), 1.967 (0.42), 2.187 (0.48), 2.204 (0.59), |
| | 2.218 (0.51), 2.235 (0.40), 2.253 (0.70), 2.279 (14.28), 2.330 (0.87), 2.518 (1.31), 2.522 |
| | (0.80), 3.310 (0.80), 3.320 (1.11), 3.529 (0.46), 3.535 (0.56), 3.542 (0.45), 3.548 (0.61), |
| | 3.554 (0.52), 3.608 (0.42), 3.627 (0.91), 3.634 (0.42), 3.644 (0.51), 3.653 (0.64), 3.674 |
| | (0.88), 3.689 (0.94), 3.701 (0.80), 3.716 (0.71), 4.194 (0.88), 4.400 (0.62), 4.413 (0.60), |
| | 4.462 (0.43), 4.477 (0.46), 5.815 (0.75), 5.833 (1.16), 5.851 (0.74), 6.958 (0.56), 6.963 |
| | (0.48), 6.965 (0.45), 6.980 (0.71), 6.985 (0.48), 6.988 (0.43), 7.110 (3.00), 7.216 (0.54), |
| | 7.220 (0.61), 7.235 (1.35), 7.239 (1.33), 7.254 (1.25), 7.258 (1.20), 7.276 (0.92), 7.280 |
| | (1.07), 7.295 (1.52), 7.298 (1.63), 7.314 (0.99), 7.316 (1.12), 7.337 (0.42), 7.376 (0.41), |
| | 7.412 (1.94), 7.416 (2.01), 7.432 (1.57), 7.436 (1.52), 7.511 (1.49), 7.515 (1.45), 7.530 |
| | (1.30), 7.534 (1.20), 8.202 (1.21), 8.219 (1.13), 8.421 (1.21), 8.440 (1.17), 8.627 (4.35). |
| 57 | 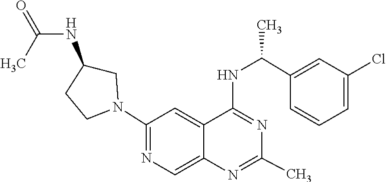 [SMILES: CC(=O)N[C@@H]1CCN(c2cc3c(N[C@H](C)c4cccc(Cl)c4)nc(C)nc3cn2)C1] |
| | N-[(3R)-1-(4-{[(1R)-1-(3-chlorophenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.574 (5.01), 1.591 (5.04), 1.824 (16.00), 1.927 |
| | (0.59), 1.940 (0.62), 1.958 (0.43), 2.178 (0.49), 2.193 (0.59), 2.209 (0.52), 2.224 (0.42), |
| | 2.332 (14.27), 3.295 (0.92), 3.306 (1.07), 3.322 (1.52), 3.333 (2.10), 3.516 (0.50), 3.521 |
| | (0.60), 3.528 (0.49), 3.534 (0.65), 3.541 (0.57), 3.554 (0.43), 3.594 (0.42), 3.612 (0.92), |
| | 3.630 (0.52), 3.637 (0.64), 3.657 (1.00), 3.673 (0.96), 3.684 (0.82), 3.700 (0.74), 4.391 |
| | (0.63), 4.404 (0.62), 5.579 (0.67), 5.597 (0.99), 5.616 (0.65), 7.039 (2.97), 7.271 (0.65), |
| | 7.276 (1.04), 7.280 (0.76), 7.290 (1.08), 7.295 (1.65), 7.299 (1.18), 7.338 (1.22), 7.357 |
| | (2.68), 7.376 (1.81), 7.383 (1.28), 7.386 (2.21), 7.390 (1.33), 7.402 (0.49), 7.405 (0.78), |
| | 7.409 (0.44), 7.476 (1.61), 7.480 (2.41), 8.198 (1.20), 8.214 (1.17), 8.317 (1.13), 8.337 |
| | (1.09), 8.636 (4.44). |
| 58 | 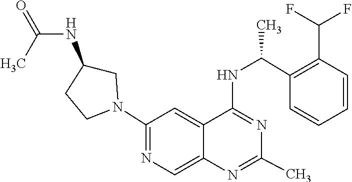 [SMILES: CC(=O)N[C@@H]1CCN(c2cc3c(N[C@H](C)c4ccccc4C(F)F)nc(C)nc3cn2)C1] |
| | N-{(3R)-1-[4-({(1RS)-1-[2-(difluoromethyl)phenyl]ethyl}amino)-2-methylpyrido[3,4- |
| | d]pyrimidin-6-yl]pyrrolidin-3-yl}acetamide |
| | ¹H-NMR (400 MHz, DMSO-d6) δ [ppm]: 1.521 (0.41), 1.590 (5.02), 1.608 (5.07), 1.828 |
| | (10.89), 1.833 (10.01), 1.930 (0.53), 1.947 (0.58), 1.961 (0.44), 2.181 (0.49), 2.197 (0.61), |
| | 2.214 (0.56), 2.230 (0.43), 2.292 (16.00), 2.314 (0.89), 2.322 (0.73), 2.326 (0.89), 2.332 |
| | (0.66), 2.518 (3.01), 2.522 (1.94), 2.539 (0.41), 2.664 (0.57), 2.668 (0.77), 2.673 (0.56), |
| | 3.314 (1.28), 3.528 (0.45), 3.541 (0.52), 3.548 (0.53), 3.566 (0.47), 3.586 (0.53), 3.604 |
| | (0.68), 3.615 (0.67), 3.622 (0.55), 3.630 (0.48), 3.645 (0.65), 3.661 (0.99), 3.672 (0.58), |
| | 3.678 (0.65), 3.689 (0.77), 3.705 (0.43), 4.406 (0.67), 5.517 (0.49), 5.535 (0.74), 5.552 |
| | (0.49), 7.045 (1.85), 7.051 (1.86), 7.343 (0.68), 7.362 (1.58), 7.381 (1.02), 7.493 (0.74), |
| | 7.512 (1.37), 7.531 (0.77), 7.546 (1.52), 7.565 (1.24), 7.613 (0.54), 7.640 (1.59), 7.660 |
| | (1.19), 7.752 (0.98), 7.891 (0.49), 8.198 (0.85), 8.205 (0.90), 8.215 (0.87), 8.221 (0.80), |
| | 8.563 (1.25), 8.580 (1.20), 8.607 (4.83). |
| 59 | 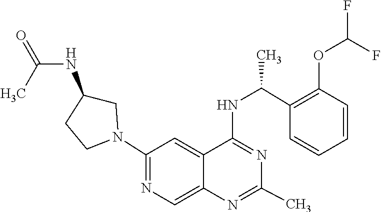 [SMILES: CC(=O)N[C@@H]1CCN(c2cc3c(N[C@H](C)c4ccccc4OC(F)F)nc(C)nc3cn2)C1] |
| | N-{(3R)-1-[4-({(1RS)-1-[2-(difluoromethoxy)phenyl]ethyl}amino)-2-methylpyrido[3,4- |
| | d]pyrimidin-6-yl]pyrrolidin-3-yl}acetamide |
| | ¹H-NMR (400 MHz, DMSO-d6) δ [ppm]: 1.535 (6.28), 1.553 (6.48), 1.821 (0.97), 1.829 |
| | (11.89), 1.834 (11.79), 1.918 (0.42), 1.933 (0.65), 1.947 (0.69), 1.963 (0.50), 2.185 (0.61), |
| | 2.201 (0.75), 2.217 (0.65), 2.233 (0.50), 2.253 (0.53), 2.278 (16.00), 2.322 (0.78), 2.326 |
| | (0.87), 2.330 (0.80), 2.518 (2.91), 2.522 (1.89), 2.664 (0.51), 2.668 (0.69), 2.673 (0.50), |
| | 3.189 (0.40), 3.359 (0.78), 3.535 (0.50), 3.549 (0.58), 3.556 (0.59), 3.573 (0.54), 3.580 |
| | (0.44), 3.593 (0.59), 3.610 (0.84), 3.627 (0.85), 3.636 (0.53), 3.644 (0.45), 3.652 (0.98), |
| | 3.669 (0.93), 3.679 (0.65), 3.687 (0.71), 3.698 (0.69), 3.714 (0.48), 4.404 (0.71), 4.416 |
| | (0.68), 5.786 (0.65), 5.804 (0.96), 5.819 (0.64), 7.098 (2.24), 7.102 (2.29), 7.139 (2.16), |
| | 7.158 (1.54), 7.178 (2.08), 7.196 (0.77), 7.214 (1.83), 7.233 (1.28), 7.272 (1.40), 7.276 |
| | (1.50), 7.291 (1.51), 7.296 (1.65), 7.310 (0.77), 7.315 (0.81), 7.321 (2.36), 7.329 (1.99), |
| | 7.508 (1.98), 7.511 (3.18), 7.527 (1.57), 8.202 (1.04), 8.207 (1.09), 8.219 (1.03), 8.224 |
| | (0.99), 8.366 (1.52), 8.385 (1.45), 8.621 (5.55). |
| 60 | 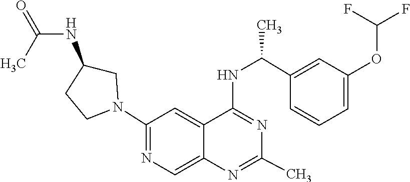 [SMILES: CC(=O)N[C@@H]1CCN(c2cc3c(N[C@H](C)c4cccc(OC(F)F)c4)nc(C)nc3cn2)C1] |
| | N-{(3R)-1-[4-({(1R)-1-[3-(difluoromethoxy)phenyl]ethyl}amino)-2-methylpyrido[3,4- |
| | d]pyrimidin-6-yl]pyrrolidin-3-yl}acetamide |
| | ¹H-NMR (400 MHz, DMSO-d6) δ [ppm]: 1.580 (5.04), 1.598 (5.07), 1.822 (16.00), 1.925 |
| | (0.64), 1.938 (0.67), 1.957 (0.47), 2.176 (0.55), 2.193 (0.64), 2.208 (0.57), 2.224 (0.44), |
| | 2.333 (14.07), 2.523 (1.16), 3.294 (0.78), 3.303 (0.92), 3.523 (0.62), 3.536 (0.68), 3.542 |
| | (0.60), 3.556 (0.44), 3.592 (0.43), 3.609 (0.98), 3.628 (0.57), 3.635 (0.69), 3.652 (1.07), |
| | 3.668 (1.01), 3.679 (0.88), 3.695 (0.77), 4.375 (0.42), 4.390 (0.69), 4.402 (0.68), 4.416 |
| | (0.40), 5.595 (0.73), 5.613 (1.06), 5.632 (0.71), 7.029 (2.75), 7.044 (4.24), 7.214 (3.56), |
| | 7.240 (2.13), 7.297 (1.12), 7.317 (1.82), 7.363 (1.95), 7.383 (2.63), 7.400 (1.90), 7.402 |
| | (1.35), 8.191 (1.28), 8.208 (1.25), 8.318 (1.32), 8.337 (1.26), 8.632 (4.57). |
| 61 | 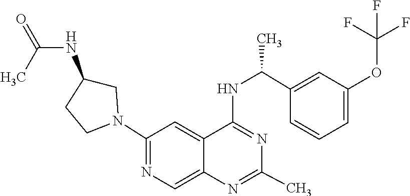 [SMILES: CC(=O)N[C@@H]1CCN(c2cc3c(N[C@H](C)c4cccc(OC(F)(F)F)c4)nc(C)nc3cn2)C1] |
| | N-{(3R)-1-[2-methyl-4-({(1R)-1-[3-(trifluoromethoxy)phenyl]ethyl}amino)pyrido[3,4- |
| | d]pyrimidin-6-yl]pyrrolidin-3-yl}acetamide |
| | ¹H-NMR (400 MHz, DMSO-d6) δ [ppm]: 1.228 (0.42), 1.593 (5.29), 1.611 (5.33), 1.823 |
| | (16.00), 1.926 (0.66), 1.940 (0.69), 1.957 (0.50), 2.178 (0.54), 2.194 (0.66), 2.210 (0.59), |
| | 2.226 (0.45), 2.320 (14.52), 2.522 (0.89), 3.292 (0.76), 3.303 (0.88), 3.320 (1.15), 3.511 |
| | (0.40), 3.524 (0.65), 3.537 (0.70), 3.544 (0.63), 3.557 (0.46), 3.591 (0.46), 3.609 (1.03), |
| | 3.627 (0.58), 3.634 (0.71), 3.653 (1.12), 3.668 (1.06), 3.679 (0.91), 3.694 (0.81), 4.377 |
| | (0.43), 4.390 (0.72), 4.403 (0.71), 4.416 (0.41), 5.590 (0.79), 5.608 (1.18), 5.626 (0.78), |
| | 7.034 (3.32), 7.222 (0.99), 7.409 (2.00), 7.456 (4.33), 7.469 (3.49), 8.191 (1.34), 8.207 |
| | (1.32), 8.345 (1.39), 8.364 (1.33), 8.635 (4.79). |
| 62 | 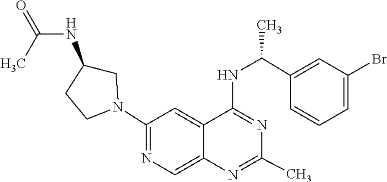 [SMILES: CC(=O)N[C@@H]1CCN(c2cc3c(N[C@H](C)c4cccc(Br)c4)nc(C)nc3cn2)C1] |
| | N-[(3R)-1-(4-{[(1R)-1-(3-bromophenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.570 (4.63), 1.588 (4.64), 1.822 (16.00), 1.926 |
| | (0.53), 1.940 (0.56), 2.179 (0.43), 2.195 (0.52), 2.210 (0.46), 2.323 (0.82), 2.333 (14.09), |
| | 2.518 (2.32), 2.523 (1.54), 2.665 (0.49), 2.669 (0.70), 2.673 (0.48), 3.294 (0.70), 3.304 |
| | (0.78), 3.518 (0.42), 3.523 (0.51), 3.530 (0.41), 3.537 (0.55), 3.543 (0.49), 3.612 (0.84), |
| | 3.630 (0.46), 3.638 (0.57), 3.658 (0.91), 3.674 (0.88), 3.684 (0.75), 3.700 (0.67), 4.391 |
| | (0.55), 4.404 (0.55), 5.568 (0.62), 5.586 (0.92), 5.604 (0.60), 7.035 (2.73), 7.278 (1.19), |
| | 7.297 (2.51), 7.316 (1.93), 7.412 (1.45), 7.418 (1.44), 7.427 (1.59), 7.430 (1.65), 7.435 |
| | (1.01), 7.437 (0.91), 7.446 (1.12), 7.620 (1.47), 7.624 (2.43), 7.629 (1.32), 8.191 (1.10), |
| | 8.207 (1.07), 8.311 (1.12), 8.330 (1.08), 8.635 (3.98). |
| 63 | 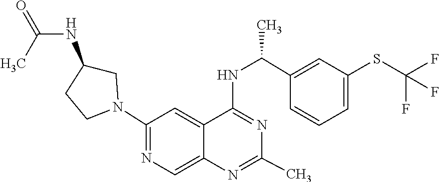 [SMILES: CC(=O)N[C@@H]1CCN(c2cc3c(N[C@H](C)c4cccc(SC(F)(F)F)c4)nc(C)nc3cn2)C1] |
| | N-[(3R)-1-(2-methyl-4-{[(1R)-1-{3- |
| | [(trifluoromethyl)sulfanyl]phenyl}ethyl]amino}pyrido[3,4-d]pyrimidin-6-yl)pyrrolidin-3- |
| | yl]acetamide |
| | ¹H-NMR (400 MHz, DMSO-d6) δ [ppm]: 1.602 (4.56), 1.620 (4.56), 1.824 (16.00), 1.929 |
| | (0.54), 1.942 (0.57), 2.179 (0.44), 2.196 (0.53), 2.211 (0.48), 2.309 (14.08), 2.518 (0.55), |
| | 3.295 (0.93), 3.305 (1.12), 3.322 (1.80), 3.520 (0.46), 3.525 (0.55), 3.532 (0.45), 3.539 |
| | (0.59), 3.545 (0.53), 3.610 (0.85), 3.628 (0.48), 3.636 (0.59), 3.653 (0.94), 3.669 (0.89), |
| | 3.680 (0.76), 3.696 (0.68), 4.392 (0.57), 4.404 (0.56), 5.574 (0.61), 5.592 (0.93), 5.610 |
| | (0.60), 7.040 (2.76), 7.482 (0.95), 7.501 (2.52), 7.520 (1.86), 7.566 (1.45), 7.585 (0.87), |
| | 7.650 (1.38), 7.670 (1.11), 7.775 (2.10), 8.200 (1.12), 8.216 (1.10), 8.378 (1.03), 8.398 |
| | (1.00), 8.630 (4.17). |
| 64 | 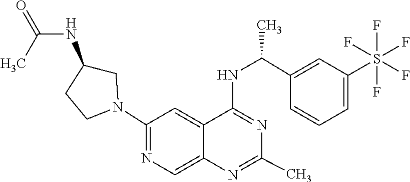 [SMILES: CC(=O)N[C@@H]1CCN(c2cc3c(N[C@H](C)c4cccc(S(F)(F)(F)(F)F)c4)nc(C)nc3cn2)C1] |
| | N-{(3R)-1-[2-methyl-4-({(1R)-1-[3-(pentafluoro-lambda⁶- |
| | sulfanyl)phenyl]ethyl}amino)pyrido[3,4-d]pyrimidin-6-yl]pyrrolidin-3-yl}acetamide |
| | ¹H-NMR (400 MHz, DMSO-d6) δ [ppm]: 1.619 (4.92), 1.637 (5.02), 1.823 (16.00), 1.928 |
| | (0.63), 1.942 (0.67), 1.959 (0.48), 2.181 (0.51), 2.197 (0.63), 2.213 (0.57), 2.228 (0.44), |
| | 2.301 (0.53), 2.314 (14.18), 2.326 (1.05), 2.331 (0.76), 2.518 (3.69), 2.522 (2.51), 2.539 |
| | (0.63), 2.665 (0.48), 2.669 (0.67), 2.673 (0.49), 3.293 (2.07), 3.303 (2.66), 3.499 (0.61), |
| | 3.512 (0.65), 3.525 (0.84), 3.538 (0.87), 3.544 (0.78), 3.557 (0.61), 3.587 (0.57), 3.606 |
| | (1.10), 3.623 (0.65), 3.631 (0.74), 3.652 (1.05), 3.668 (1.08), 3.678 (0.93), 3.694 (0.84), |
| | 4.377 (0.42), 4.390 (0.68), 4.403 (0.68), 4.417 (0.40), 5.574 (0.74), 5.593 (1.14), 5.610 |
| | (0.74), 7.016 (3.14), 7.559 (0.65), 7.579 (1.43), 7.598 (0.95), 7.717 (1.52), 7.737 (1.25), |
| | 7.749 (1.31), 7.752 (1.35), 7.769 (1.03), 7.773 (1.10), 7.970 (2.24), 8.194 (1.31), 8.201 |
| | (1.35), 8.218 (1.29), 8.410 (1.31), 8.429 (1.25), 8.631 (4.66). |
| 65 | 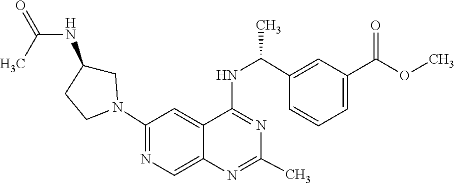 [SMILES: COC(=O)c1cccc([C@@H](C)Nc2nc(C)nc3cnc(N4CC[C@@H](NC(C)=O)C4)cc23)c1] |
| | methyl 3-[(1R)-1-({6-[(3R)-3-acetamidopyrrolidin-1-yl]-2-methylpyrido[3,4-d]pyrimidin- |
| | 4-yl}amino)ethyl]benzoate |
| | ¹H-NMR (400 MHz, DMSO-d6) δ [ppm]: 1.602 (4.14), 1.620 (4.16), 1.822 (13.62), 1.927 |
| | (0.52), 1.940 (0.56), 2.179 (0.45), 2.195 (0.52), 2.209 (0.47), 2.323 (12.05), 2.522 (1.41), |
| | 2.539 (1.86), 2.668 (0.49), 3.294 (0.68), 3.303 (0.78), 3.523 (0.52), 3.537 (0.56), 3.544 |
| | (0.49), 3.610 (0.82), 3.628 (0.47), 3.636 (0.56), 3.655 (0.89), 3.671 (0.82), 3.681 (0.71), |
| | 3.697 (0.61), 3.834 (16.00), 4.391 (0.59), 4.405 (0.56), 5.615 (0.61), 5.633 (0.92), 5.651 |
| | (0.61), 7.049 (2.59), 7.468 (1.04), 7.488 (2.28), 7.507 (1.29), 7.714 (1.22), 7.733 (1.04), |
| | 7.814 (1.36), 7.817 (0.96), 7.830 (0.87), 7.833 (1.22), 8.063 (2.31), 8.196 (1.08), 8.213 |
| | (1.08), 8.390 (1.13), 8.410 (1.08), 8.627 (3.81). |
| 66 | 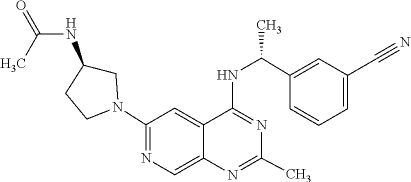 [SMILES: CC(=O)N[C@@H]1CCN(c2cc3c(N[C@H](C)c4cccc(C#N)c4)nc(C)nc3cn2)C1] |
| | N-[(3R)-1-(4-{[(1R)-1-(3-cyanophenyl)ethyl]amino}-2-methylpyrido[3,4-d]pyrimidin-6- |
| | yl)pyrrolidin-3-yl]acetamide |
| | ¹H-NMR (400 MHz, DMSO-d6) δ [ppm]: 1.593 (4.68), 1.611 (4.79), 1.629 (0.52), 1.710 |
| | (0.45), 1.718 (0.44), 1.727 (1.17), 1.734 (0.44), 1.743 (0.46), 1.822 (16.00), 1.927 (0.56), |
| | 1.941 (0.59), 1.959 (0.41), 2.179 (0.45), 2.195 (0.55), 2.210 (0.49), 2.321 (13.87), 2.518 |
| | (2.26), 2.522 (1.44), 2.664 (0.42), 2.668 (0.56), 2.673 (0.40), 2.997 (0.46), 3.003 (0.85), |
| | 3.013 (0.84), 3.020 (0.45), 3.297 (0.70), 3.307 (0.85), 3.520 (0.44), 3.525 (0.54), 3.532 |
| | (0.46), 3.538 (0.60), 3.545 (0.54), 3.558 (0.40), 3.615 (0.87), 3.632 (0.48), 3.640 (0.61), |
| | 3.661 (0.89), 3.676 (0.90), 3.687 (0.77), 3.703 (0.69), 4.388 (0.59), 4.402 (0.59), 5.594 |
| | (0.67), 5.612 (0.99), 5.630 (0.65), 7.034 (2.79), 7.528 (1.08), 7.547 (2.46), 7.566 (1.58), |
| | 7.694 (1.06), 7.698 (1.71), 7.701 (1.18), 7.713 (0.92), 7.717 (1.35), 7.720 (0.91), 7.757 |
| | (1.37), 7.777 (1.15), 7.882 (2.49), 8.188 (1.14), 8.205 (1.11), 8.339 (1.19), 8.358 (1.15), |
| | 8.637 (4.13). |
| 67 | 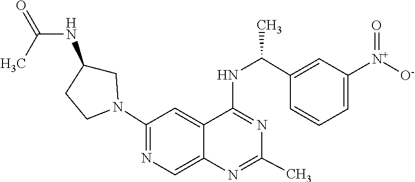 [SMILES: CC(=O)N[C@@H]1CCN(c2cc3c(N[C@H](C)c4cccc([N+](=O)[O-])c4)nc(C)nc3cn2)C1] |
| | N-[(3R)-1-(2-methyl-4-{[(1R)-1-(3-nitrophenyl)ethyl]amino}pyrido[3,4-d]pyrimidin-6- |
| | yl)pyrrolidin-3-yl]acetamide |
| | ¹H-NMR (400 MHz, DMSO-d6) δ [ppm]: 0.792 (0.56), 0.809 (0.61), 0.816 (0.60), 0.899 |
| | (0.60), 1.628 (5.47), 1.646 (5.53), 1.820 (16.00), 1.911 (0.41), 1.928 (0.70), 1.942 (0.75), |
| | 1.960 (0.53), 2.180 (0.59), 2.195 (0.72), 2.211 (0.65), 2.226 (0.57), 2.316 (14.51), 2.665 |
| | (0.61), 3.154 (1.32), 3.167 (1.35), 3.298 (0.85), 3.308 (1.24), 3.525 (0.68), 3.538 (0.76), |
| | 3.545 (0.68), 3.558 (0.51), 3.593 (0.48), 3.612 (1.08), 3.630 (0.64), 3.637 (0.78), 3.659 |
| | (1.08), 3.674 (1.10), 3.685 (0.95), 3.701 (0.84), 4.376 (0.48), 4.389 (0.78), 4.402 (0.80), |
| | 4.415 (0.47), 5.643 (0.82), 5.661 (1.24), 5.679 (0.82), 5.754 (2.93), 7.040 (3.46), 7.615 |
| | (1.32), 7.635 (2.68), 7.655 (1.61), 7.894 (1.69), 7.913 (1.47), 8.082 (1.29), 8.086 (1.31), |
| | 8.102 (1.20), 8.106 (1.23), 8.194 (1.41), 8.211 (1.41), 8.306 (2.76), 8.433 (1.47), 8.452 |
| | (1.42), 8.633 (4.92). |
| 68 | 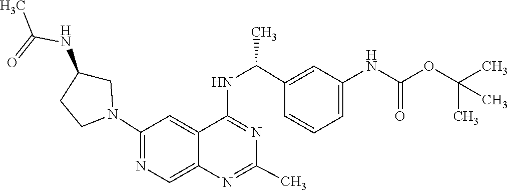 [SMILES: CC(=O)N[C@@H]1CCN(c2cc3c(N[C@H](C)c4cccc(NC(=O)OC(C)(C)C)c4)nc(C)nc3cn2)C1] |
| | tert-butyl {3-[(1RS)-1-({6-[(3R)-3-acetamidopyrrolidin-1-yl]-2-methylpyrido[3,4- |
| | d]pyrimidin-4-yl}amino)ethyl]phenyl}carbamate |
| | ¹H-NMR (400 MHz, DMSO-d6) δ [ppm]: 1.446 (16.00), 1.465 (1.36), 1.552 (2.00), 1.570 |
| | (1.97), 1.818 (6.91), 2.323 (0.46), 2.327 (0.62), 2.343 (6.19), 2.669 (0.47), 3.295 (0.57), |
| | 3.308 (0.77), 5.568 (0.42), 7.028 (0.57), 7.048 (0.75), 7.063 (1.10), 7.167 (0.52), 7.186 |
| | (0.99), 7.206 (0.61), 7.267 (0.49), 7.565 (0.75), 8.186 (0.53), 8.201 (0.52), 8.283 (0.59), |
| | 8.303 (0.56), 8.621 (1.97), 9.295 (0.63). |
| 69 | 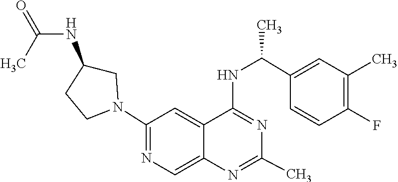 [SMILES: CC(=O)N[C@@H]1CCN(c2cc3c(N[C@H](C)c4ccc(F)c(C)c4)nc(C)nc3cn2)C1] |
| | N-[(3R)-1-(4-{[(1R)-1-(4-fluoro-3-methylphenyl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (3.67), 1.555 (4.88), 1.573 (4.91), 1.822 |
| | (16.00), 1.917 (0.57), 1.930 (0.59), 1.948 (0.42), 2.169 (0.47), 2.185 (0.60), 2.210 (6.57), |
| | 2.215 (6.83), 2.327 (0.46), 2.340 (14.08), 2.518 (1.09), 2.523 (0.71), 3.295 (0.69), 3.306 |
| | (0.76), 3.323 (1.26), 3.535 (0.56), 3.549 (0.64), 3.555 (0.60), 3.570 (0.66), 3.591 (0.97), |
| | 3.609 (0.52), 3.616 (0.56), 3.625 (0.82), 3.641 (0.95), 3.652 (0.79), 3.668 (0.70), 4.388 |
| | (0.59), 4.402 (0.59), 5.556 (0.62), 5.574 (0.92), 5.592 (0.62), 7.029 (2.86), 7.050 (1.09), |
| | 7.071 (1.50), 7.074 (1.31), 7.095 (1.30), 7.242 (0.52), 7.248 (0.63), 7.254 (0.60), 7.261 |
| | (0.83), 7.269 (0.57), 7.276 (0.46), 7.282 (0.52), 7.320 (0.96), 7.326 (0.88), 7.339 (0.98), |
| | 7.345 (0.86), 8.191 (1.16), 8.208 (1.15), 8.258 (1.19), 8.278 (1.15), 8.624 (4.26). |
| 70 | 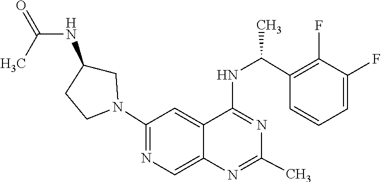 [SMILES: CC(=O)N[C@@H]1CCN(c2cc3c(N[C@H](C)c4cccc(F)c4F)nc(C)nc3cn2)C1] |
| | N-[(3R)-1-(4-{[(1R)-1-(2,3-difluorophenyl)ethyl]amino}-2-methylpyrido[3,4-d]pyrimidin- |
| | 6-yl)pyrrolidin-3-yl]acetamide |
| | ¹H-NMR (400 MHz, DMSO-d6) δ [ppm]: 1.107 (1.28), 1.598 (4.77), 1.616 (4.76), 1.827 |
| | (16.00), 1.932 (0.56), 1.945 (0.59), 1.963 (0.42), 2.183 (0.46), 2.199 (0.55), 2.214 (0.49), |
| | 2.298 (14.32), 2.327 (0.47), 2.518 (1.58), 2.523 (1.04), 2.669 (0.42), 3.301 (0.84), 3.311 |
| | (1.04), 3.525 (0.44), 3.530 (0.54), 3.537 (0.44), 3.544 (0.59), 3.550 (0.53), 3.619 (0.88), |
| | 3.637 (0.49), 3.645 (0.61), 3.664 (0.97), 3.680 (0.93), 3.691 (0.79), 3.706 (0.72), 4.395 |
| | (0.60), 4.408 (0.59), 5.749 (0.70), 5.767 (1.09), 5.785 (0.70), 7.079 (2.88), 7.148 (0.69), |
| | 7.161 (0.72), 7.164 (0.68), 7.168 (0.57), 7.181 (0.50), 7.184 (0.46), 7.249 (0.83), 7.253 |
| | (0.74), 7.265 (1.06), 7.272 (1.04), 7.278 (0.96), 7.282 (0.88), 7.291 (0.46), 7.298 (0.72), |
| | 7.302 (0.55), 8.198 (1.13), 8.215 (1.11), 8.391 (1.12), 8.409 (1.08), 8.636 (4.18). |
| 71 | 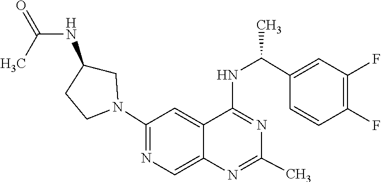 [SMILES: CC(=O)N[C@@H]1CCN(c2cc3c(N[C@H](C)c4ccc(F)c(F)c4)nc(C)nc3cn2)C1] |
| | N-[(3R)-1-(4-{[(1R)-1-(3,4-difluorophenyl)ethyl]amino}-2-methylpyrido[3,4-d]pyrimidin- |
| | 6-yl)pyrrolidin-3-yl]acetamide |
| | ¹H-NMR (400 MHz, DMSO-d6) δ [ppm]: 1.567 (5.02), 1.584 (5.07), 1.822 (16.00), 1.925 |
| | (0.58), 1.938 (0.61), 1.956 (0.43), 2.175 (0.52), 2.192 (0.57), 2.207 (0.50), 2.335 (14.42), |
| | 2.518 (0.92), 2.523 (0.59), 3.292 (0.72), 3.302 (0.79), 3.318 (1.04), 3.514 (0.45), 3.520 |
| | (0.56), 3.528 (0.45), 3.533 (0.61), 3.540 (0.54), 3.554 (0.40), 3.591 (0.40), 3.609 (0.91), |
| | 3.616 (0.43), 3.627 (0.50), 3.634 (0.63), 3.653 (1.02), 3.668 (0.96), 3.679 (0.82), 3.694 |
| | (0.74), 4.387 (0.61), 4.399 (0.61), 5.563 (0.64), 5.582 (0.97), 5.600 (0.64), 7.024 (2.97), |
| | 7.246 (0.44), 7.257 (0.49), 7.267 (0.68), 7.271 (0.65), 7.278 (0.64), 7.345 (0.77), 7.366 |
| | (1.26), 7.371 (0.87), 7.387 (0.63), 7.393 (1.24), 7.414 (0.56), 7.446 (0.59), 7.451 (0.59), |
| | 7.466 (0.64), 7.471 (0.66), 7.476 (0.68), 7.481 (0.62), 7.495 (0.61), 7.501 (0.58), 8.189 |
| | (1.17), 8.207 (1.15), 8.287 (1.20), 8.307 (1.16), 8.636 (4.36). |
| 72 | 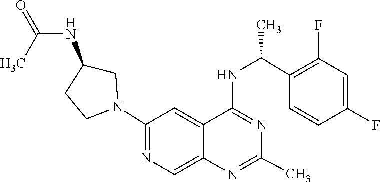 [SMILES: CC(=O)N[C@@H]1CCN(c2cc3c(N[C@H](C)c4ccc(F)cc4F)nc(C)nc3cn2)C1] |
| | N-[(3R)-1-(4-{[(1R)-1-(2,4-difluorophenyl)ethyl]amino}-2-methylpyrido[3,4-d]pyrimidin- |
| | 6-yl)pyrrolidin-3-yl]acetamide |
| | ¹H-NMR (400 MHz, DMSO-d6) δ [ppm]: 1.568 (4.94), 1.586 (4.90), 1.826 (16.00), 1.930 |
| | (0.59), 1.943 (0.62), 1.961 (0.43), 2.179 (0.49), 2.196 (0.60), 2.210 (0.53), 2.227 (0.41), |
| | 2.304 (14.25), 2.518 (0.40), 3.299 (0.94), 3.309 (1.12), 3.327 (1.72), 3.520 (0.50), 3.525 |
| | (0.60), 3.533 (0.50), 3.539 (0.64), 3.545 (0.57), 3.558 (0.42), 3.596 (0.41), 3.615 (0.91), |
| | 3.632 (0.51), 3.640 (0.63), 3.658 (1.03), 3.674 (0.96), 3.685 (0.83), 3.701 (0.74), 4.392 |
| | (0.64), 4.406 (0.63), 5.708 (0.68), 5.727 (1.04), 5.744 (0.68), 7.021 (0.45), 7.026 (0.50), |
| | 7.042 (0.94), 7.048 (1.04), 7.067 (3.36), 7.171 (0.67), 7.177 (0.67), 7.194 (0.82), 7.198 |
| | (0.92), 7.200 (0.92), 7.204 (0.78), 7.221 (0.67), 7.227 (0.64), 7.462 (0.52), 7.479 (0.65), |
| | 7.484 (1.05), 7.501 (1.05), 7.506 (0.63), 7.522 (0.48), 8.202 (1.19), 8.218 (1.17), 8.338 |
| | (1.08), 8.357 (1.04), 8.631 (4.42). |
| 73 | 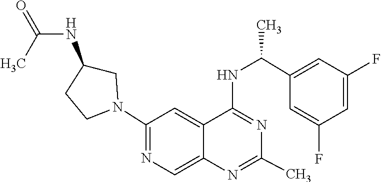 [SMILES: CC(=O)N[C@@H]1CCN(c2cc3c(N[C@H](C)c4cc(F)cc(F)c4)nc(C)nc3cn2)C1] |
| | N-[(3R)-1-(4-{[(1RS)-1-(3,5-difluorophenyl)ethyl]amino}-2-methylpyrido[3,4-d]pyrimidin- |
| | 6-yl)pyrrolidin-3-yl]acetamide |
| | ¹H-NMR (400 MHz, DMSO-d6) δ [ppm]: 1.570 (5.60), 1.588 (5.72), 1.806 (0.43), 1.822 |
| | (11.43), 1.825 (11.30), 1.909 (0.42), 1.926 (0.68), 1.940 (0.71), 1.957 (0.52), 2.176 (0.51), |
| | 2.193 (0.63), 2.210 (0.59), 2.224 (0.64), 2.330 (16.00), 2.518 (2.99), 2.522 (2.03), 2.539 |
| | (0.44), 2.668 (0.51), 3.228 (0.40), 3.297 (0.64), 3.311 (1.17), 3.525 (0.48), 3.538 (0.54), |
| | 3.545 (0.57), 3.552 (0.51), 3.559 (0.50), 3.565 (0.49), 3.571 (0.44), 3.584 (0.49), 3.599 |
| | (0.82), 3.616 (0.81), 3.638 (0.76), 3.654 (0.68), 3.664 (0.94), 3.679 (0.92), 3.689 (0.52), |
| | 3.705 (0.46), 4.392 (0.72), 4.405 (0.71), 5.577 (0.60), 5.595 (0.89), 5.612 (0.59), 7.027 |
| | (2.09), 7.032 (2.07), 7.061 (0.76), 7.067 (0.57), 7.079 (0.69), 7.084 (1.49), 7.090 (1.09), |
| | 7.108 (0.92), 7.113 (0.92), 7.127 (2.04), 7.144 (2.22), 8.191 (0.86), 8.198 (0.94), 8.207 |
| | (0.93), 8.214 (0.86), 8.299 (1.32), 8.319 (1.29), 8.646 (4.96). |
| 74 | 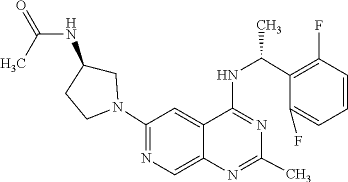 [SMILES: CC(=O)N[C@@H]1CCN(c2cc3c(N[C@H](C)c4c(F)cccc4F)nc(C)nc3cn2)C1] |
| | N-[(3R)-1-(4-{[(1RS)-1-(2,6-difluorophenyl)ethyl]amino}-2-methylpyrido[3,4-d]pyrimidin- |
| | 6-yl)pyrrolidin-3-yl]acetamide |
| | ¹H-NMR (400 MHz, DMSO-d6) δ [ppm]: 1.675 (0.48), 1.694 (6.06), 1.712 (6.03), 1.824 |
| | (11.80), 1.830 (11.86), 1.904 (0.43), 1.918 (0.64), 1.935 (0.71), 1.949 (0.55), 2.174 (0.66), |
| | 2.190 (0.79), 2.206 (0.72), 2.221 (0.58), 2.266 (16.00), 2.518 (4.62), 2.523 (3.13), 3.181 |
| | (0.59), 3.193 (0.49), 3.307 (1.09), 3.513 (0.46), 3.518 (0.49), 3.531 (0.72), 3.537 (0.75), |
| | 3.550 (0.73), 3.575 (0.41), 3.585 (0.42), 3.594 (0.78), 3.603 (0.92), 3.611 (0.64), 3.621 |
| | (0.78), 3.629 (1.02), 3.638 (0.89), 3.643 (0.92), 3.654 (1.14), 3.664 (0.70), 3.670 (0.61), |
| | 3.680 (0.58), 4.396 (0.82), 4.410 (0.82), 5.631 (0.57), 5.647 (0.81), 5.659 (0.58), 6.986 |
| | (1.83), 7.006 (3.90), 7.027 (2.18), 7.119 (4.09), 7.245 (0.40), 7.261 (0.93), 7.266 (0.78), |
| | 7.282 (1.48), 7.298 (0.72), 7.303 (0.80), 8.196 (0.97), 8.203 (1.07), 8.213 (1.09), 8.219 |
| | (0.99), 8.389 (1.39), 8.404 (1.38), 8.603 (5.70). |
| 75 | 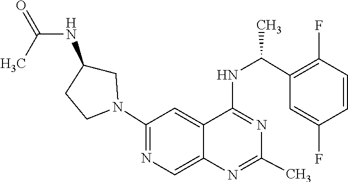 [SMILES: CC(=O)N[C@@H]1CCN(c2cc3c(N[C@H](C)c4cc(F)ccc4F)nc(C)nc3cn2)C1] |
| | N-[(3R)-1-(4-{[(1RS)-1-(2,5-difluorophenyl)ethyl]amino}-2-methylpyrido[3,4-d]pyrimidin- |
| | 6-yl)pyrrolidin-3-yl]acetamide |
| | ¹H-NMR (400 MHz, DMSO-d6) δ [ppm]: 1.571 (5.96), 1.589 (5.92), 1.825 (11.54), 1.829 |
| | (11.66), 1.931 (0.66), 1.944 (0.71), 1.962 (0.50), 2.181 (0.49), 2.197 (0.61), 2.214 (0.57), |
| | 2.230 (0.41), 2.304 (16.00), 2.322 (0.65), 2.326 (0.79), 2.332 (0.54), 2.518 (2.80), 2.522 |
| | (1.79), 2.664 (0.51), 2.668 (0.68), 2.673 (0.50), 3.182 (0.48), 3.196 (0.42), 3.306 (0.74), |
| | 3.356 (0.84), 3.532 (0.44), 3.546 (0.48), 3.552 (0.49), 3.560 (0.50), 3.573 (0.47), 3.580 |
| | (0.44), 3.593 (0.41), 3.605 (0.87), 3.623 (0.87), 3.631 (0.54), 3.646 (0.78), 3.661 (0.68), |
| | 3.672 (1.00), 3.687 (1.00), 3.698 (0.55), 3.713 (0.47), 4.398 (0.71), 4.411 (0.69), 5.732 |
| | (0.53), 5.749 (0.78), 5.763 (0.52), 7.052 (2.13), 7.056 (2.17), 7.094 (0.54), 7.107 (0.61), |
| | 7.116 (1.06), 7.126 (0.77), 7.136 (0.78), 7.145 (0.50), 7.215 (0.91), 7.226 (0.98), 7.239 |
| | (1.60), 7.250 (1.79), 7.259 (1.05), 7.262 (1.18), 7.273 (1.18), 7.278 (0.50), 8.194 (0.84), |
| | 8.202 (0.92), 8.211 (0.91), 8.219 (0.82), 8.311 (1.30), 8.329 (1.27), 8.644 (5.22). |
| 76 | 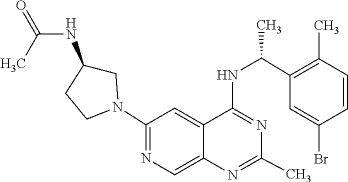 [SMILES: CC(=O)N[C@@H]1CCN(c2cc3c(N[C@H](C)c4cc(Br)ccc4C)nc(C)nc3cn2)C1] |
| | N-[(3R)-1-(4-{[(1R)-1-(5-bromo-2-methylphenyl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (0.57), 1.230 (0.44), 1.512 (4.70), 1.530 |
| | (4.81), 1.826 (16.00), 1.933 (0.57), 1.947 (0.60), 1.964 (0.42), 2.185 (0.46), 2.202 (0.56), |
| | 2.217 (0.52), 2.233 (0.42), 2.313 (14.71), 2.327 (0.91), 2.332 (0.57), 2.456 (11.15), 2.518 |
| | (2.45), 2.523 (1.61), 2.540 (4.13), 2.665 (0.44), 2.669 (0.60), 2.673 (0.42), 3.159 (2.51), |
| | 3.171 (2.66), 3.304 (0.92), 3.314 (1.31), 3.521 (0.48), 3.527 (0.56), 3.534 (0.49), 3.540 |
| | (0.60), 3.547 (0.53), 3.559 (0.41), 3.603 (0.40), 3.621 (0.91), 3.639 (0.51), 3.647 (0.65), |
| | 3.672 (0.83), 3.687 (0.94), 3.698 (0.83), 3.714 (0.73), 4.098 (0.44), 4.111 (0.44), 4.399 |
| | (0.60), 4.413 (0.59), 5.582 (0.72), 5.600 (1.09), 5.618 (0.71), 7.037 (2.97), 7.112 (1.75), |
| | 7.133 (2.24), 7.273 (1.66), 7.278 (1.66), 7.293 (1.26), 7.298 (1.29), 7.603 (2.61), 7.609 |
| | (2.56), 8.199 (1.19), 8.216 (1.17), 8.382 (1.19), 8.400 (1.17), 8.624 (4.37). |
| 77 | 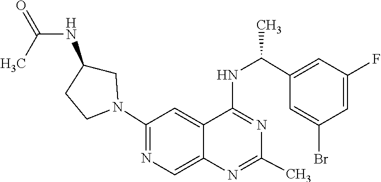 [SMILES: CC(=O)N[C@@H]1CCN(c2cc3c(N[C@H](C)c4cc(F)cc(Br)c4)nc(C)nc3cn2)C1] |
| | N-[(3R)-1-(4-{[(1R)-1-(3-bromo-5-fluorophenyl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (0.44), 1.568 (4.65), 1.586 (4.68), 1.823 |
| | (16.00), 1.929 (0.54), 1.942 (0.58), 1.960 (0.40), 2.181 (0.44), 2.197 (0.53), 2.213 (0.47), |
| | 2.323 (0.75), 2.333 (14.19), 2.518 (2.61), 2.523 (1.72), 2.540 (15.00), 2.665 (0.47), 2.669 |
| | (0.65), 2.673 (0.46), 3.298 (0.77), 3.308 (1.00), 3.521 (0.44), 3.526 (0.54), 3.533 (0.46), |
| | 3.539 (0.58), 3.546 (0.51), 3.616 (0.86), 3.634 (0.47), 3.642 (0.60), 3.665 (0.81), 3.680 |
| | (0.89), 3.692 (0.77), 3.708 (0.68), 4.392 (0.58), 4.406 (0.58), 5.556 (0.65), 5.575 (0.96), |
| | 5.592 (0.63), 7.022 (2.77), 7.287 (0.95), 7.291 (0.79), 7.311 (0.91), 7.315 (0.77), 7.393 |
| | (0.77), 7.398 (1.11), 7.403 (0.82), 7.414 (0.77), 7.418 (1.12), 7.424 (0.81), 7.485 (2.58), |
| | 8.192 (1.12), 8.209 (1.12), 8.307 (1.14), 8.326 (1.11), 8.646 (4.12). |
| 78 | 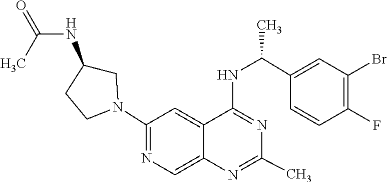 [SMILES: CC(=O)N[C@@H]1CCN(c2cc3c(N[C@H](C)c4ccc(F)c(Br)c4)nc(C)nc3cn2)C1] |
| | N-[(3R)-1-(4-{[(1R)-1-(3-bromo-4-fluorophenyl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.568 (4.73), 1.586 (4.81), 1.822 (16.00), 1.907 |
| | (0.41), 1.926 (0.58), 1.939 (0.61), 1.957 (0.43), 2.178 (0.54), 2.193 (0.58), 2.209 (0.50), |
| | 2.324 (0.57), 2.338 (13.75), 2.354 (0.40), 2.518 (0.71), 2.523 (0.44), 3.292 (0.71), 3.302 |
| | (0.79), 3.319 (1.05), 3.514 (0.45), 3.520 (0.55), 3.527 (0.44), 3.533 (0.60), 3.540 (0.54), |
| | 3.591 (0.41), 3.609 (0.89), 3.616 (0.45), 3.627 (0.51), 3.634 (0.63), 3.654 (0.94), 3.670 |
| | (0.92), 3.681 (0.79), 3.696 (0.70), 4.388 (0.60), 4.402 (0.60), 5.553 (0.66), 5.571 (0.96), |
| | 5.589 (0.64), 7.014 (2.79), 7.309 (1.35), 7.331 (2.88), 7.352 (1.79), 7.449 (0.72), 7.454 |
| | (0.79), 7.461 (0.83), 7.466 (0.85), 7.470 (0.69), 7.476 (0.66), 7.482 (0.59), 7.488 (0.59), |
| | 7.737 (1.29), 7.742 (1.28), 7.754 (1.32), 7.759 (1.27), 8.192 (1.19), 8.208 (1.17), 8.300 |
| | (1.07), 8.319 (1.05), 8.635 (4.15). |
| 79 | 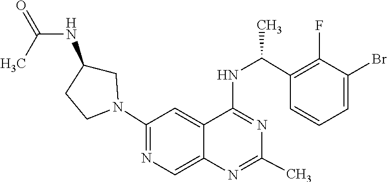 [SMILES: CC(=O)N[C@@H]1CCN(c2cc3c(N[C@H](C)c4cccc(Br)c4F)nc(C)nc3cn2)C1] |
| | N-[(3R)-1-(4-{[(1R)-1-(3-bromo-2-fluorophenyl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (3.08), 1.516 (0.53), 1.591 (4.64), 1.608 |
| | (4.63), 1.826 (16.00), 1.931 (0.57), 1.944 (0.59), 1.962 (0.42), 2.182 (0.46), 2.198 (0.55), |
| | 2.212 (0.49), 2.295 (14.72), 2.518 (2.80), 2.523 (1.81), 2.540 (1.18), 2.674 (0.52), 3.301 |
| | (0.91), 3.311 (1.28), 3.523 (0.46), 3.529 (0.57), 3.537 (0.44), 3.542 (0.59), 3.549 (0.53), |
| | 3.619 (0.86), 3.637 (0.49), 3.645 (0.62), 3.665 (0.94), 3.680 (0.92), 3.691 (0.79), 3.707 |
| | (0.70), 4.394 (0.60), 4.407 (0.59), 5.730 (0.69), 5.748 (1.07), 5.759 (0.75), 5.765 (0.69), |
| | 7.079 (2.86), 7.100 (0.94), 7.120 (1.93), 7.140 (1.07), 7.433 (0.63), 7.437 (0.71), 7.454 |
| | (1.19), 7.469 (0.60), 7.473 (0.59), 7.547 (0.79), 7.551 (0.79), 7.564 (0.91), 7.567 (1.43), |
| | 7.571 (0.81), 7.584 (0.76), 7.587 (0.68), 8.197 (1.15), 8.214 (1.13), 8.394 (1.02), 8.412 |
| | (1.00), 8.634 (4.27). |
| 80 | 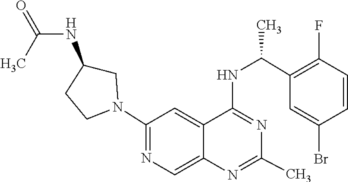 [SMILES: CC(=O)N[C@@H]1CCN(c2cc3c(N[C@H](C)c4cc(Br)ccc4F)nc(C)nc3cn2)C1] |
| | N-[(3R)-1-(4-{[(1R)-1-(5-bromo-2-fluorophenyl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (9.00), 1.569 (4.52), 1.587 (4.58), 1.827 |
| | (16.00), 1.934 (0.55), 1.947 (0.59), 1.966 (0.41), 2.187 (0.44), 2.203 (0.54), 2.218 (0.49), |
| | 2.295 (0.74), 2.307 (13.93), 2.323 (0.59), 2.327 (0.66), 2.332 (0.46), 2.518 (2.07), 2.523 |
| | (1.37), 2.540 (0.81), 2.669 (0.56), 3.307 (1.06), 3.527 (0.45), 3.532 (0.54), 3.540 (0.44), |
| | 3.546 (0.58), 3.552 (0.51), 3.565 (0.40), 3.606 (0.42), 3.623 (0.87), 3.642 (0.50), 3.649 |
| | (0.61), 3.674 (0.79), 3.689 (0.89), 3.700 (0.77), 3.716 (0.68), 4.400 (0.58), 4.413 (0.58), |
| | 5.716 (0.68), 5.735 (1.02), 5.753 (0.67), 5.758 (0.56), 7.048 (2.73), 7.173 (1.30), 7.195 |
| | (1.63), 7.199 (1.39), 7.220 (1.49), 7.450 (0.72), 7.456 (0.81), 7.461 (0.81), 7.468 (0.82), |
| | 7.471 (0.72), 7.478 (0.78), 7.483 (0.67), 7.490 (0.66), 7.611 (1.13), 7.617 (1.10), 7.628 |
| | (1.17), 7.634 (1.05), 8.202 (1.12), 8.219 (1.10), 8.341 (1.04), 8.360 (1.01), 8.647 (4.10). |
| 81 | 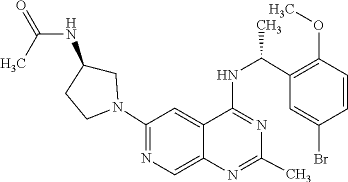 [SMILES: COc1ccc(Br)cc1[C@@H](C)Nc1nc(C)nc2cnc(N3CC[C@@H](NC(C)=O)C3)cc12] |
| | N-[(3R)-1-(4-{[(1R)-1-(5-bromo-2-methoxyphenyl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (3.45), 1.479 (5.06), 1.496 (5.19), 1.828 |
| | (16.00), 1.936 (0.62), 1.950 (0.66), 1.968 (0.48), 2.190 (0.48), 2.207 (0.58), 2.221 (0.54), |
| | 2.238 (0.43), 2.294 (14.10), 2.518 (6.47), 2.523 (4.42), 2.540 (8.23), 3.312 (1.26), 3.538 |
| | (0.62), 3.551 (0.64), 3.571 (0.43), 3.611 (0.43), 3.629 (0.97), 3.647 (0.56), 3.655 (0.68), |
| | 3.678 (0.93), 3.693 (0.99), 3.705 (0.85), 3.720 (0.76), 3.772 (0.54), 3.870 (15.44), 4.407 |
| | (0.66), 4.418 (0.66), 5.815 (0.76), 5.833 (1.01), 5.852 (0.74), 6.982 (2.63), 7.004 (3.02), |
| | 7.089 (3.08), 7.363 (1.61), 7.370 (1.74), 7.385 (1.34), 7.391 (1.55), 7.460 (2.98), 7.466 |
| | (2.52), 8.208 (1.32), 8.225 (1.49), 8.232 (1.47), 8.252 (1.22), 8.636 (4.57). |
| 82 | 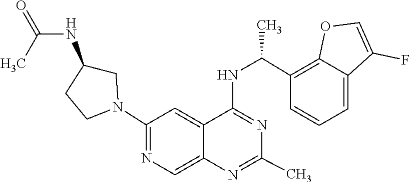 [SMILES: CC(=O)N[C@@H]1CCN(c2cc3c(N[C@H](C)c4cccc5c(F)coc45)nc(C)nc3cn2)C1] |
| | N-[(3R)-1-(4-{[(1R)-1-(3-fluoro-1-benzofuran-7-yl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (0.44), 1.682 (4.75), 1.700 (4.81), 1.825 |
| | (16.00), 1.927 (0.57), 1.941 (0.59), 1.959 (0.43), 2.179 (0.45), 2.195 (0.56), 2.210 (0.50), |
| | 2.226 (0.40), 2.264 (14.03), 2.518 (2.28), 2.523 (1.48), 2.674 (0.41), 3.299 (0.82), 3.309 |
| | (1.11), 3.523 (0.45), 3.528 (0.55), 3.536 (0.44), 3.542 (0.59), 3.548 (0.53), 3.616 (0.86), |
| | 3.634 (0.49), 3.642 (0.61), 3.660 (0.97), 3.675 (0.91), 3.687 (0.80), 3.703 (0.69), 4.394 |
| | (0.60), 4.407 (0.59), 6.045 (0.67), 6.063 (1.01), 6.081 (0.66), 7.109 (2.81), 7.290 (1.23), |
| | 7.309 (2.75), 7.328 (1.79), 7.423 (1.69), 7.442 (1.26), 7.550 (1.75), 7.553 (1.70), 7.570 |
| | (1.55), 7.572 (1.42), 8.196 (1.17), 8.212 (1.16), 8.317 (2.68), 8.328 (2.66), 8.430 (1.17), |
| | 8.450 (1.14), 8.632 (4.18). |
| 83 | 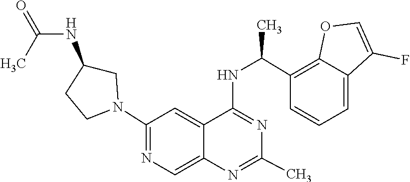 [SMILES: CC(=O)N[C@@H]1CCN(c2cc3c(N[C@@H](C)c4cccc5c(F)coc45)nc(C)nc3cn2)C1] |
| | N-[(3R)-1-(4-{[(1S)-1-(3-fluoro-1-benzofuran-7-yl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (16.00), 1.683 (4.68), 1.701 (4.68), 1.827 |
| | (15.55), 1.925 (0.56), 1.938 (0.58), 1.956 (0.42), 2.178 (0.45), 2.193 (0.54), 2.209 (0.49), |
| | 2.262 (13.62), 2.518 (1.84), 2.523 (1.21), 3.308 (1.01), 3.549 (0.56), 3.556 (0.41), 3.563 |
| | (0.64), 3.568 (0.59), 3.584 (0.73), 3.603 (0.94), 3.621 (0.51), 3.629 (0.53), 3.642 (0.81), |
| | 3.657 (0.91), 3.668 (0.78), 3.684 (0.69), 4.195 (0.51), 4.395 (0.59), 4.409 (0.58), 6.042 |
| | (0.66), 6.060 (0.99), 6.078 (0.66), 7.103 (2.76), 7.289 (1.17), 7.307 (2.66), 7.326 (1.75), |
| | 7.419 (1.66), 7.438 (1.22), 7.549 (1.72), 7.552 (1.67), 7.569 (1.49), 7.572 (1.39), 8.201 |
| | (1.18), 8.217 (1.16), 8.314 (2.60), 8.326 (2.59), 8.430 (1.16), 8.449 (1.12), 8.633 (4.10). |
| 84 | 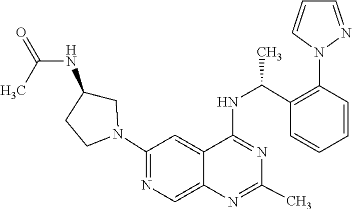 [SMILES: CC(=O)N[C@@H]1CCN(c2cc3c(N[C@H](C)c4ccccc4-n4cccn4)nc(C)nc3cn2)C1] |
| | N-{(3R)-1-[2-methyl-4-({(1RS)-1-[2-(1H-pyrazol-1-yl)phenyl]ethyl}amino)pyrido[3,4- |
| | d]pyrimidin-6-yl]pyrrolidin-3-yl}acetamide |
| | ¹H-NMR (400 MHz, DMSO-d6) δ [ppm]: 1.525 (5.26), 1.542 (5.29), 1.711 (3.48), 1.719 |
| | (3.47), 1.727 (9.46), 1.735 (3.48), 1.744 (3.62), 1.827 (9.95), 1.834 (10.24), 1.929 (0.59), |
| | 1.945 (0.61), 1.960 (0.47), 2.180 (0.62), 2.203 (16.00), 2.229 (0.50), 2.518 (3.65), 2.522 |
| | (2.34), 2.988 (2.50), 2.998 (3.78), 3.005 (6.92), 3.008 (3.01), 3.014 (7.05), 3.021 (3.58), |
| | 3.031 (2.43), 3.302 (0.56), 3.356 (0.65), 3.527 (0.47), 3.540 (0.53), 3.547 (0.55), 3.560 |
| | (0.48), 3.572 (0.40), 3.584 (0.51), 3.601 (0.79), 3.617 (0.78), 3.643 (0.89), 3.659 (0.75), |
| | 3.668 (0.74), 3.682 (0.76), 3.693 (0.54), 3.709 (0.45), 4.407 (0.67), 5.455 (0.60), 5.468 |
| | (0.87), 5.473 (0.88), 5.486 (0.59), 6.563 (2.58), 6.568 (3.48), 6.573 (2.53), 7.048 (1.95), |
| | 7.054 (1.97), 7.302 (0.60), 7.306 (0.95), 7.321 (2.67), 7.326 (3.57), 7.331 (1.75), 7.344 |
| | (1.83), 7.348 (1.77), 7.364 (0.72), 7.367 (0.71), 7.404 (0.86), 7.409 (0.82), 7.424 (1.28), |
| | 7.441 (0.66), 7.445 (0.61), 7.658 (1.77), 7.678 (1.52), 7.775 (3.29), 7.779 (3.27), 8.196 |
| | (0.90), 8.203 (1.04), 8.213 (3.88), 8.219 (3.96), 8.414 (1.38), 8.432 (1.32), 8.583 (5.10). |
| 85 | 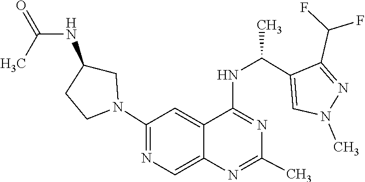 [SMILES: CC(=O)N[C@@H]1CCN(c2cc3c(N[C@H](C)c4cn(C)nc4C(F)F)nc(C)nc3cn2)C1] |
| | N-{(3R)-1-[4-({(1RS)-1-[3-(difluoromethyl)-1-methyl-1H-pyrazol-4-yl]ethyl}amino)-2- |
| | methylpyrido[3,4-d]pyrimidin-6-yl]pyrrolidin-3-yl}acetamide |
| | ¹H-NMR (400 MHz, DMSO-d6) δ [ppm]: 1.107 (7.13), 1.232 (0.72), 1.551 (4.33), 1.568 |
| | (4.31), 1.815 (16.00), 1.904 (0.48), 1.922 (0.52), 1.935 (0.42), 2.160 (0.49), 2.176 (0.60), |
| | 2.191 (0.54), 2.208 (0.41), 2.373 (14.38), 2.518 (1.90), 2.522 (1.21), 3.263 (0.61), 3.271 |
| | (0.64), 3.290 (0.72), 3.299 (0.79), 3.311 (0.46), 3.501 (0.47), 3.514 (0.57), 3.521 (0.52), |
| | 3.533 (0.49), 3.561 (0.44), 3.580 (0.95), 3.597 (0.55), 3.606 (0.73), 3.618 (0.60), 3.625 |
| | (0.77), 3.634 (0.71), 3.644 (0.51), 3.653 (0.47), 3.660 (0.46), 3.841 (9.56), 4.193 (0.68), |
| | 4.374 (0.61), 4.388 (0.60), 5.628 (0.64), 5.646 (0.96), 5.664 (0.63), 6.894 (0.74), 6.898 |
| | (0.76), 6.957 (2.89), 7.030 (1.37), 7.033 (1.42), 7.165 (0.68), 7.168 (0.67), 7.816 (3.47), |
| | 8.140 (1.33), 8.158 (1.29), 8.181 (1.27), 8.198 (1.20), 8.619 (4.56). |
| 86 | 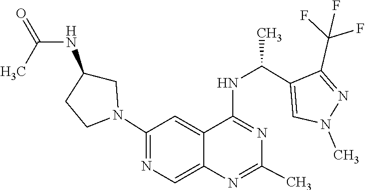 [SMILES: CC(=O)N[C@@H]1CCN(c2cc3c(N[C@H](C)c4cn(C)nc4C(F)(F)F)nc(C)nc3cn2)C1] |
| | N-{(3R)-1-[2-methyl-4-({(1RS)-1-[1-methyl-3-(trifluoromethyl)-1H-pyrazol-4- |
| | yl]ethyl}amino)pyrido[3,4-d]pyrimidin-6-yl]pyrrolidin-3-yl}acetamide |
| | ¹H-NMR (400 MHz, DMSO-d6) δ [ppm]: 0.901 (0.73), 1.052 (0.71), 1.070 (0.45), 1.539 |
| | (4.98), 1.556 (4.97), 1.599 (0.84), 1.610 (0.91), 1.620 (0.93), 1.644 (0.80), 1.659 (0.66), |
| | 1.672 (0.55), 1.727 (0.81), 1.815 (16.00), 1.895 (0.82), 1.910 (1.39), 1.924 (1.08), 1.939 |
| | (0.60), 2.065 (0.91), 2.160 (0.54), 2.177 (0.66), 2.194 (0.64), 2.209 (0.48), 2.354 (15.79), |
| | 2.378 (0.54), 2.518 (2.80), 2.522 (1.82), 2.616 (0.73), 2.642 (0.71), 3.005 (0.59), 3.014 |
| | (0.62), 3.230 (0.69), 3.245 (1.13), 3.261 (1.14), 3.273 (0.61), 3.280 (0.65), 3.290 (1.01), |
| | 3.300 (0.75), 3.454 (0.67), 3.469 (1.17), 3.485 (0.85), 3.500 (0.56), 3.513 (0.65), 3.525 |
| | (0.65), 3.532 (1.26), 3.544 (0.84), 3.556 (1.04), 3.578 (1.08), 3.596 (0.64), 3.604 (0.81), |
| | 3.624 (0.90), 3.636 (0.66), 3.639 (0.64), 3.651 (0.59), 3.885 (11.80), 4.357 (0.43), 4.373 |
| | (0.65), 4.387 (0.62), 5.635 (0.69), 5.652 (1.03), 5.670 (0.68), 6.951 (3.36), 7.942 (3.22), |
| | 8.181 (1.72), 8.186 (1.93), 8.197 (1.61), 8.205 (1.58), 8.626 (4.93). |
| 87 | 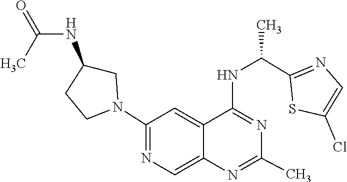 [SMILES: CC(=O)N[C@@H]1CCN(c2cc3c(N[C@H](C)c4ncc(Cl)s4)nc(C)nc3cn2)C1] |
| | N-[(3R)-1-(4-{[(1RS)-1-(5-chloro-1,3-thiazol-2-yl)ethyl]amino}-2-methylpyrido[3,4- |
| | d]pyrimidin-6-yl)pyrrolidin-3-yl]acetamide |
| | ¹H-NMR (400 MHz, DMSO-d6) δ [ppm]: 1.107 (10.04), 1.230 (0.45), 1.707 (5.65), 1.725 |
| | (6.79), 1.743 (1.11), 1.817 (16.00), 1.899 (0.45), 1.919 (0.71), 1.933 (0.73), 1.951 (0.52), |
| | 2.171 (0.56), 2.187 (0.68), 2.203 (0.60), 2.219 (0.47), 2.408 (15.15), 2.518 (2.87), 2.523 |
| | (2.00), 2.539 (0.72), 2.987 (0.61), 2.997 (0.97), 3.003 (1.71), 3.013 (1.71), 3.020 (0.94), |
| | 3.029 (0.59), 3.281 (0.71), 3.290 (0.74), 3.308 (0.96), 3.514 (0.49), 3.528 (0.63), 3.535 |
| | (0.58), 3.547 (0.55), 3.571 (0.45), 3.590 (0.89), 3.620 (0.81), 3.635 (0.90), 3.647 (1.01), |
| | 3.658 (0.61), 3.673 (0.44), 4.192 (0.68), 4.367 (0.46), 4.381 (0.74), 4.394 (0.73), 4.407 |
| | (0.42), 5.750 (0.89), 5.759 (0.40), 5.768 (1.38), 5.786 (0.89), 7.000 (3.21), 7.768 (4.91), |
| | 8.187 (1.32), 8.205 (1.29), 8.596 (1.50), 8.615 (1.44), 8.691 (4.88). |
| 88 | 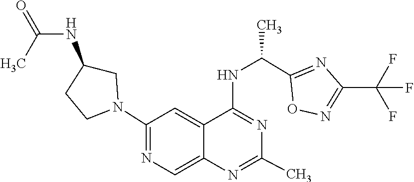 [SMILES: CC(=O)N[C@@H]1CCN(c2cc3c(N[C@H](C)c4nc(C(F)(F)F)no4)nc(C)nc3cn2)C1] |
| | N-{(3R)-1-[2-methyl-4-({(1RS)-1-[3-(trifluoromethyl)-1,2,4-oxadiazol-5- |
| | yl]ethyl}amino)pyrido[3,4-d]pyrimidin-6-yl]pyrrolidin-3-yl}acetamide |
| | ¹H-NMR (400 MHz, DMSO-d6) δ [ppm]: 1.107 (0.57), 1.230 (0.74), 1.760 (5.65), 1.777 |
| | (5.66), 1.819 (16.00), 1.929 (0.60), 1.942 (0.63), 1.960 (0.45), 2.179 (0.49), 2.196 (0.60), |
| | 2.212 (0.54), 2.230 (0.51), 2.271 (13.34), 2.332 (0.43), 2.518 (2.21), 2.522 (1.37), 3.304 |
| | (0.84), 3.523 (0.44), 3.535 (0.57), 3.543 (0.51), 3.554 (0.49), 3.578 (0.43), 3.596 (0.89), |
| | 3.615 (0.52), 3.622 (0.56), 3.632 (0.50), 3.646 (0.79), 3.660 (0.84), 3.672 (0.64), 4.383 |
| | (0.65), 4.396 (0.64), 5.747 (0.83), 5.764 (1.17), 5.781 (0.83), 6.960 (2.89), 8.193 (0.96), |
| | 8.208 (0.94), 8.699 (4.16), 8.765 (1.16), 8.780 (1.13). |
| 89 | 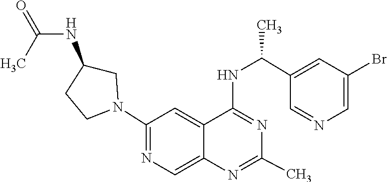 [SMILES: CC(=O)N[C@@H]1CCN(c2cc3c(N[C@H](C)c4cncc(Br)c4)nc(C)nc3cn2)C1] |
| | N-[(3R)-1-(4-{[(1R)-1-(5-bromopyridin-3-yl)ethyl]amino}-2-methylpyrido[3,4-d]pyrimidin- |
| | 6-yl)pyrrolidin-3-yl]acetamide |
| | ¹H-NMR (400 MHz, DMSO-d6) δ [ppm]: 0.901 (0.62), 1.035 (0.47), 1.052 (1.01), 1.070 |
| | (0.61), 1.231 (0.86), 1.617 (4.91), 1.634 (4.95), 1.711 (1.00), 1.719 (1.01), 1.727 (2.63), |
| | 1.735 (1.01), 1.744 (1.05), 1.751 (0.61), 1.821 (16.00), 1.926 (0.59), 1.940 (0.61), 1.958 |
| | (0.44), 2.065 (0.81), 2.084 (1.69), 2.178 (0.51), 2.195 (0.56), 2.210 (0.51), 2.322 (0.88), |
| | 2.327 (1.35), 2.337 (14.85), 2.518 (4.29), 2.523 (2.75), 2.664 (0.71), 2.669 (0.96), 2.673 |
| | (0.71), 2.988 (0.73), 2.998 (1.01), 3.004 (1.97), 3.013 (1.92), 3.020 (1.03), 3.031 (0.66), |
| | 3.295 (0.69), 3.306 (0.79), 3.519 (0.46), 3.524 (0.54), 3.537 (0.59), 3.544 (0.54), 3.557 |
| | (0.41), 3.594 (0.41), 3.611 (0.89), 3.630 (0.49), 3.637 (0.62), 3.661 (0.83), 3.676 (0.93), |
| | 3.687 (0.81), 3.703 (0.69), 4.388 (0.61), 4.402 (0.59), 5.561 (0.69), 5.579 (1.06), 5.597 |
| | (0.68), 6.997 (2.87), 8.076 (1.50), 8.080 (2.72), 8.085 (1.55), 8.187 (1.15), 8.204 (1.13), |
| | 8.334 (1.23), 8.353 (1.18), 8.575 (3.34), 8.581 (3.22), 8.645 (4.47), 8.652 (3.09), 8.656 |
| | (2.92). |
| 90 | 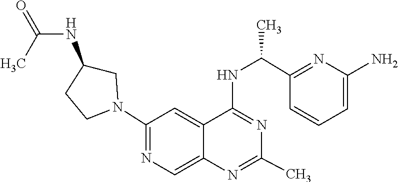 [SMILES: CC(=O)N[C@@H]1CCN(c2cc3c(N[C@H](C)c4cccc(N)n4)nc(C)nc3cn2)C1] |
| | N-[(3R)-1-(4-{[(1R)-1-(6-aminopyridin-2-yl)ethyl]amino}-2-methylpyrido[3,4-d]pyrimidin- |
| | 6-yl)pyrrolidin-3-yl]acetamide |
| | ¹H-NMR (400 MHz, DMSO-d6) δ [ppm]: 1.525 (5.27), 1.543 (5.57), 1.751 (0.74), 1.821 |
| | (16.00), 1.919 (0.64), 1.933 (0.66), 1.951 (0.48), 2.173 (0.50), 2.189 (0.61), 2.204 (0.53), |
| | 2.220 (0.41), 2.322 (13.91), 2.336 (0.88), 2.518 (5.04), 2.523 (3.73), 2.550 (0.67), 2.665 |
| | (0.53), 2.669 (0.74), 2.673 (0.53), 3.293 (0.80), 3.303 (0.93), 3.525 (0.60), 3.538 (0.66), |
| | 3.545 (0.57), 3.557 (0.44), 3.588 (0.43), 3.607 (0.93), 3.625 (0.54), 3.632 (0.64), 3.651 |
| | (1.02), 3.666 (0.96), 3.678 (0.83), 3.693 (0.76), 4.376 (0.40), 4.389 (0.64), 4.403 (0.65), |
| | 5.411 (0.75), 5.430 (1.05), 5.448 (0.75), 5.871 (3.91), 6.280 (2.15), 6.300 (2.21), 6.497 |
| | (2.07), 6.516 (2.12), 7.083 (2.93), 7.273 (1.66), 7.292 (2.12), 7.312 (1.44), 8.190 (1.29), |
| | 8.206 (1.29), 8.228 (1.27), 8.248 (1.20), 8.628 (4.19). |
| 91 | 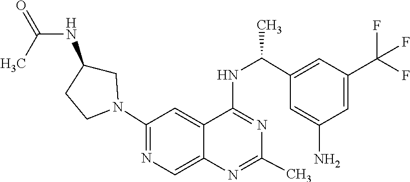 [SMILES: CC(=O)N[C@@H]1CCN(c2cc3c(N[C@H](C)c4cc(N)cc(C(F)(F)F)c4)nc(C)nc3cn2)C1] |
| | N-{(3R)-1-[4-({(1R)-1-[3-amino-5-(trifluoromethyl)phenyl]ethyl}amino)-2- |
| | methylpyrido[3,4-d]pyrimidin-6-yl]pyrrolidin-3-yl}acetamide |
| | ¹H-NMR (400 MHz, DMSO-d6) δ [ppm]: 1.232 (0.42), 1.542 (4.20), 1.560 (4.31), 1.613 |
| | (0.73), 1.619 (0.53), 1.752 (2.54), 1.779 (0.74), 1.806 (2.06), 1.821 (16.00), 1.831 (0.70), |
| | 1.924 (0.59), 1.937 (0.59), 1.955 (0.43), 1.978 (5.58), 2.054 (1.02), 2.174 (0.42), 2.191 |
| | (0.53), 2.205 (0.46), 2.226 (0.74), 2.323 (0.77), 2.327 (1.08), 2.332 (1.00), 2.342 (13.51), |
| | 2.352 (0.83), 2.363 (0.57), 2.518 (3.90), 2.523 (2.75), 2.665 (0.66), 2.669 (0.94), 2.673 |
| | (0.65), 3.280 (0.73), 3.291 (0.83), 3.308 (1.27), 3.513 (0.43), 3.519 (0.53), 3.532 (0.57), |
| | 3.539 (0.51), 3.608 (0.82), 3.626 (0.46), 3.634 (0.57), 3.652 (0.90), 3.667 (0.87), 3.678 |
| | (0.74), 3.694 (0.66), 4.386 (0.57), 4.399 (0.57), 5.516 (0.43), 5.532 (0.71), 5.560 (3.39), |
| | 6.698 (2.15), 6.832 (1.90), 6.866 (2.06), 7.050 (2.66), 8.189 (1.10), 8.206 (1.07), 8.292 |
| | (0.49), 8.310 (0.48), 8.631 (4.11). |

### EXAMPLE 92

### N-[(3R)-1-(4-{[1-(3-aminophenyl)ethyl]amino}-2-methylpyrido[3,4-d]pyrimidin-6-yl)pyrrolidin-3-yl]acetamide (mixture of stereoisomers)

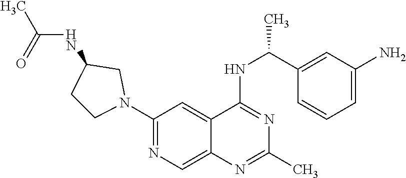 [SMILES: CC(=O)N[C@@H]1CCN(c2cc3c(N[C@H](C)c4cccc(N)c4)nc(C)nc3cn2)C1]

To Example 68 (25.0 mg, 49.4 μmol) was added 4M HCl in dioxane (3.1 ml) followed by MeOH (3 ml). The reaction was stirred at RT for 3 h and concentrated. The titled compound (9 mg, 43%) was isolated after preparative HPLC (basic method).

¹H-NMR (400 MHz, DMSO-d6) δ [ppm]: 1.514 (4.94), 1.532 (5.01), 1.819 (16.00), 1.914 (0.59), 1.928 (0.64), 1.944 (0.49), 2.167 (0.57), 2.184 (0.74), 2.199 (0.62), 2.215 (0.48), 2.327 (0.77), 2.344 (15.22), 2.522 (2.32), 2.665 (0.44), 2.669 (0.61), 2.673 (0.44), 3.280 (0.42), 3.290 (0.54), 3.305 (0.88), 3.528 (0.62), 3.545 (0.57), 3.590 (0.66), 3.603 (0.73), 3.622 (0.79), 3.638 (0.76), 3.659 (0.70), 3.669 (0.61), 3.685 (0.47), 4.370 (0.45), 4.384 (0.77), 4.397 (0.73), 4.410 (0.44), 5.003 (3.73), 5.512 (0.68), 5.531 (0.99), 5.548 (0.68), 6.392 (1.18), 6.396 (1.20), 6.412 (1.25), 6.415 (1.32), 6.563 (1.45), 6.583 (1.90), 6.589 (2.20), 6.594 (2.57), 6.931 (1.52), 6.950 (2.59), 6.970 (1.28), 7.070 (2.87), 8.184 (1.30), 8.201 (1.32), 8.219 (1.48), 8.239 (1.40), 8.617 (5.00).

### EXAMPLE 93

### tert-butyl {3-[(1S)-1-({6-[(3R)-3-acetamidopyrrolidin-1-yl]-2-methylpyrido[3,4-d]pyrimidin-4-yl}amino)ethyl]phenyl}carbamate

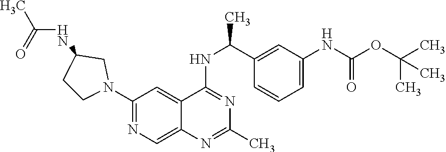 [SMILES: CC(=O)N[C@@H]1CCN(c2cc3c(N[C@@H](C)c4cccc(NC(=O)OC(C)(C)C)c4)nc(C)nc3cn2)C1]

Example 68 (116 mg, 230 μmol) was purified by chiral HPLC to give:

Example 93 (68 mg, 56%, e.e. >95%). Rt=8.51 min

Example 94 (37 mg, 33%, e.e. >95%). Rt=6.24 mins

Analytical Method: Instrument: Agilent: 1260, Aurora SFC-Module; Column: Chiralpak IC 5μ 100×4.6 mm; eluent A: CO2; eluent B: 2-propanol+0.4 vol % diethylamine; isocratic: 30% B; flow: 4 ml/min; temperature: 37.5° C.; BPR: 100 bar; UV: 280 nm

Preparative Method: Instrument: Sepiatec: Prep SFC100; Column: Chiralpak IC 5μ 250×30 mm; eluent A: CO2; eluent B: 2-propanol+0.4 vol % diethylamine; isocratic: 30% B; flow: 100 ml/min; temperature: 40° C.; BPR: 150 bar; UV: 280 nm.

### EXAMPLE 94

### tert-butyl {3-[(1R)-1-({6-[(3R)-3-acetamidopyrrolidin-1-yl]-2-methylpyrido[3,4-d]pyrimidin-4-yl}amino)ethyl]phenyl}carbamate

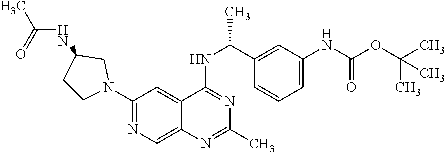 [SMILES: CC(=O)N[C@@H]1CCN(c2cc3c(N[C@H](C)c4cccc(NC(=O)OC(C)(C)C)c4)nc(C)nc3cn2)C1]

See example 93 for details.

### EXAMPLE 95

### N-[(3R)-1-(4-{[(1S)-1-(3-aminophenyl)ethyl]amino}-2-methylpyrido[3,4-d]pyrimidin-6-yl)pyrrolidin-3-yl]acetamide

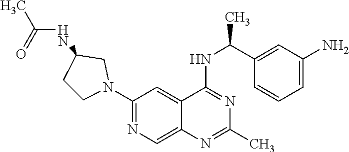 [SMILES: CC(=O)N[C@@H]1CCN(c2cc3c(N[C@@H](C)c4cccc(N)c4)nc(C)nc3cn2)C1]

Using the method described for Example 92: Example 93 gave the titled compound (12 mg, 60%) after preparative HPLC (basic method).

¹H-NMR (400 MHz, DMSO-d6) δ [ppm]: 1.593 (4.29), 1.610 (4.27), 1.822 (16.00), 1.949 (0.54), 1.963 (0.56), 1.979 (0.47), 2.190 (0.41), 2.206 (0.52), 2.222 (0.49), 2.518 (3.31), 2.523 (2.89), 2.530 (9.56), 3.316 (0.90), 3.326 (1.04), 3.344 (1.36), 3.558 (0.52), 3.572 (0.58), 3.592 (0.67), 3.611 (0.74), 3.629 (0.46), 3.637 (0.44), 3.655 (0.79), 3.670 (0.79), 3.681 (0.67), 3.697 (0.59), 4.380 (0.60), 4.392 (0.59), 5.665 (0.56), 5.684 (0.80), 5.701 (0.55), 6.468 (0.91), 6.471 (0.93), 6.487 (0.96), 6.490 (1.01), 6.590 (1.05), 6.609 (1.26), 6.619 (1.54), 6.623 (1.93), 6.988 (1.36), 7.007 (2.26), 7.026 (1.16), 7.276 (2.17), 8.209 (1.18), 8.225 (1.15), 8.726 (4.11).

### EXAMPLE 96

### N-[(3R)-1-(4-{[(1R)-1-(3-aminophenyl)ethyl]amino}-2-methylpyrido[3,4-d]pyrimidin-6-yl)pyrrolidin-3-yl]acetamide

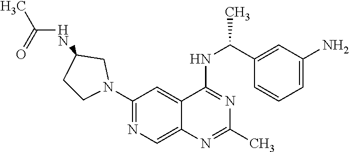 [SMILES: CC(=O)N[C@@H]1CCN(c2cc3c(N[C@H](C)c4cccc(N)c4)nc(C)nc3cn2)C1]

Using the method described for Example 92: Example 94 gave the titled compound (12 mg, 60%) after preparative HPLC (basic method).

¹H-NMR (400 MHz, DMSO-d6) δ [ppm]: 1.584 (4.03), 1.601 (4.08), 1.821 (16.00), 1.950 (0.51), 1.962 (0.55), 1.980 (0.44), 2.204 (0.49), 2.219 (0.44), 2.323 (0.51), 2.327 (0.73), 2.332 (0.52), 2.518 (3.95), 2.523 (2.49), 2.665 (0.53), 2.669 (0.76), 2.673 (0.52), 3.301 (0.83), 3.311 (1.01), 3.537 (0.47), 3.551 (0.50), 3.622 (0.67), 3.640 (0.40), 3.648 (0.46), 3.670 (0.75), 3.686 (0.80), 3.698 (0.69), 3.713 (0.60), 4.379 (0.57), 4.392 (0.56), 5.650 (0.52), 5.668 (0.75), 5.687 (0.50), 6.456 (0.90), 6.460 (0.90), 6.476 (0.94), 6.480 (0.99), 6.583 (1.08), 6.603 (1.29), 6.613 (1.51), 6.617 (1.96), 6.981 (1.37), 7.000 (2.27), 7.019 (1.17), 7.263 (1.88), 8.205 (1.10), 8.222 (1.07), 8.716 (3.74).

### EXAMPLE 97

### N-[(3R)-1-(4-{[(1R)-1-(3,5-difluorophenyl)ethyl]amino}-2-methylpyrido[3,4-d]pyrimidin-6-yl)pyrrolidin-3-yl]acetamide

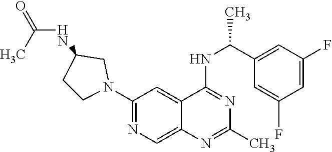 [SMILES: CC(=O)N[C@@H]1CCN(c2cc3c(N[C@H](C)c4cc(F)cc(F)c4)nc(C)nc3cn2)C1]

Example 73 (70 mg, 188 μmol) was purified by chiral HPLC to give:

Example 97 (25 mg, e.e. >95%). Rt=4.57 min

Example 98 (23 mg, e.e. >95%). Rt=5.44 mins

Analytical Method: Instrument: Thermo Fisher UltiMate 3000; Column: YMC Cellulose SB 3μ, 100×4.6; eluent A: methyl tert-butyl ether+0.1 vol % diethylamine; eluent B: ethanol; isocratic: 95% A+5% B; flow: 1.4 ml/min; temperature: 25° C.; UV: 280 nm

Preparative Method: Instrument: PrepCon Labomatic HPLC-3; Column: YMC Cellulose SB 10μ, 250×50; eluent A: methyl tert-butyl ether+0.1 vol % diethylamine; eluent B: ethanol+0.1 vol % diethylamine; isocratic: 95% A+5% B; flow: 80 ml/min; temperature: 25° C.; UV: 280 nm

¹H-NMR (400 MHz, DMSO-d6) δ [ppm]: 1.570 (4.93), 1.588 (5.00), 1.823 (16.00), 1.928 (0.57), 1.941 (0.60), 1.959 (0.43), 2.179 (0.46), 2.196 (0.55), 2.210 (0.49), 2.331 (13.92), 2.348 (0.45), 2.518 (0.65), 2.523 (0.40), 3.298 (0.66), 3.308 (0.74), 3.325 (1.11), 3.519 (0.43), 3.525 (0.53), 3.532 (0.43), 3.537 (0.58), 3.544 (0.52), 3.558 (0.40), 3.597 (0.41), 3.616 (0.87), 3.634 (0.49), 3.641 (0.61), 3.663 (0.87), 3.678 (0.91), 3.689 (0.78), 3.705 (0.69), 4.391 (0.59), 4.404 (0.58), 5.578 (0.67), 5.597 (0.98), 5.615 (0.66), 7.032 (2.76), 7.059 (0.70), 7.065 (0.52), 7.076 (0.58), 7.083 (1.35), 7.088 (1.03), 7.105 (0.75), 7.112 (0.79), 7.122 (1.56), 7.128 (1.78), 7.145 (1.92), 7.149 (1.36), 8.190 (1.12), 8.207 (1.12), 8.300 (1.12), 8.319 (1.09), 8.646 (4.19).

### EXAMPLE 98

### N-[(3R)-1-(4-{[(1S)-1-(3,5-difluorophenyl)ethyl]amino}-2-methylpyrido[3,4-d]pyrimidin-6-yl)pyrrolidin-3-yl]acetamide

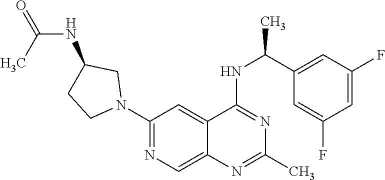 [SMILES: CC(=O)N[C@@H]1CCN(c2cc3c(N[C@@H](C)c4cc(F)cc(F)c4)nc(C)nc3cn2)C1]

See example 97 for details.

¹H-NMR (400 MHz, DMSO-d6) δ [ppm]: 1.571 (4.85), 1.589 (4.97), 1.825 (16.00), 1.907 (0.52), 1.926 (0.58), 1.940 (0.60), 1.957 (0.44), 2.175 (0.50), 2.191 (0.56), 2.206 (0.50), 2.330 (14.07), 2.518 (0.79), 2.523 (0.50), 3.311 (0.76), 3.321 (0.97), 3.551 (0.57), 3.558 (0.40), 3.565 (0.66), 3.571 (0.63), 3.583 (0.77), 3.600 (1.00), 3.618 (0.53), 3.625 (0.52), 3.638 (0.82), 3.654 (0.91), 3.665 (0.79), 3.680 (0.69), 4.395 (0.60), 4.409 (0.58), 5.574 (0.67), 5.592 (0.98), 5.610 (0.67), 7.026 (2.78), 7.059 (0.68), 7.065 (0.51), 7.076 (0.58), 7.082 (1.32), 7.088 (1.00), 7.105 (0.79), 7.111 (0.83), 7.121 (1.55), 7.126 (1.77), 7.143 (1.91), 7.148 (1.36), 8.196 (1.16), 8.214 (1.14), 8.297 (1.15), 8.316 (1.10), 8.646 (4.11).

### EXAMPLE 99

### N-[(3R)-1-(4-{[(1S)-1-(2,6-difluorophenyl)ethyl]amino}-2-methylpyrido[3,4-d]pyrimidin-6-yl)pyrrolidin-3-yl]acetamide

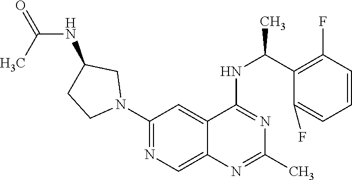 [SMILES: CC(=O)N[C@@H]1CCN(c2cc3c(N[C@@H](C)c4c(F)cccc4F)nc(C)nc3cn2)C1]

Example 74 (80 mg, 190 μmol) was purified by chiral HPLC to give:

Example 99 (25 mg, 30%, e.e. >95%). Rt=5.00 min

Example 100 (29 mg, 34%, e.e. >95%). Rt=3.31 mins

Analytical Method: Instrument: Thermo Fisher UltiMate 3000; Column: YMC Cellulose SB 3μ, 100×4.6; eluent A: methyl tert-butyl ether+0.1 vol % diethylamine; eluent B: ethanol; isocratic: 95% A+5% B; flow: 1.4 ml/min; temperature: 25° C.; UV: 280 nm

Preparative Method: Instrument: PrepCon Labomatic HPLC-3; Column: YMC Cellulose SB 10μ, 250×50; eluent A: methyl tert-butyl ether+0.1 vol % diethylamine; eluent B: ethanol+0.1 vol % diethylamine; isocratic: 95% A+5% B; flow: 80 ml/min; temperature: 25° C.; UV: 280 nm

¹H-NMR (400 MHz, DMSO-d6) δ [ppm]: 1.695 (4.49), 1.713 (4.49), 1.831 (16.00), 1.918 (0.59), 1.931 (0.60), 1.950 (0.44), 2.074 (1.34), 2.173 (0.47), 2.190 (0.56), 2.204 (0.51), 2.220 (0.40), 2.266 (14.03), 2.277 (0.57), 2.518 (0.72), 2.523 (0.51), 3.294 (0.69), 3.304 (0.76), 3.321 (1.07), 3.535 (0.57), 3.543 (0.40), 3.549 (0.64), 3.555 (0.58), 3.569 (0.50), 3.575 (0.49), 3.594 (0.96), 3.602 (0.41), 3.612 (0.54), 3.620 (0.60), 3.628 (0.83), 3.644 (0.96), 3.655 (0.80), 3.670 (0.69), 4.399 (0.59), 4.412 (0.58), 5.625 (0.63), 5.642 (0.89), 5.659 (0.60), 6.984 (1.40), 6.993 (0.41), 7.005 (2.91), 7.018 (0.42), 7.026 (1.65), 7.117 (2.91), 7.261 (0.71), 7.265 (0.57), 7.277 (0.48), 7.282 (1.11), 7.286 (0.49), 7.297 (0.56), 7.302 (0.63), 8.203 (1.16), 8.219 (1.13), 8.389 (1.03), 8.406 (0.98), 8.603 (4.05).

### EXAMPLE 100

### N-[(3R)-1-(4-{[(1R)-1-(2,6-difluorophenyl)ethyl]amino}-2-methylpyrido[3,4-d]pyrimidin-6-yl)pyrrolidin-3-yl]acetamide

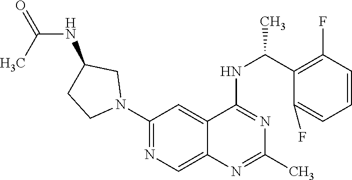 [SMILES: CC(=O)N[C@@H]1CCN(c2cc3c(N[C@H](C)c4c(F)cccc4F)nc(C)nc3cn2)C1]

See example 99 for details.

¹H-NMR (400 MHz, DMSO-d6) δ [ppm]: 1.693 (4.37), 1.711 (4.42), 1.825 (16.00), 1.922 (0.56), 1.936 (0.59), 1.954 (0.42), 2.174 (0.48), 2.191 (0.57), 2.205 (0.50), 2.221 (0.40), 2.267 (13.53), 2.283 (0.44), 2.518 (0.55), 3.299 (0.70), 3.309 (0.74), 3.326 (0.97), 3.511 (0.43), 3.517 (0.54), 3.524 (0.43), 3.530 (0.57), 3.537 (0.52), 3.603 (0.88), 3.610 (0.41), 3.621 (0.50), 3.629 (0.65), 3.637 (0.82), 3.653 (0.94), 3.665 (0.78), 3.680 (0.69), 4.395 (0.57), 4.409 (0.57), 5.631 (0.61), 5.649 (0.87), 5.666 (0.58), 6.984 (1.36), 7.005 (2.87), 7.026 (1.61), 7.118 (2.89), 7.259 (0.68), 7.264 (0.56), 7.280 (1.07), 7.297 (0.52), 7.301 (0.58), 8.197 (1.13), 8.214 (1.11), 8.387 (1.05), 8.403 (1.01), 8.603 (4.11).

### EXAMPLE 101

### N-[(3R)-1-(4-{[(1R)-1-(2,5-difluorophenyl)ethyl]amino}-2-methylpyrido[3,4-d]pyrimidin-6-yl)pyrrolidin-3-yl]acetamide

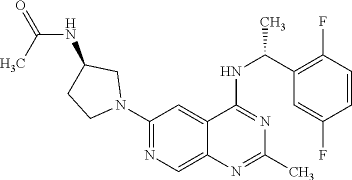 [SMILES: CC(=O)N[C@@H]1CCN(c2cc3c(N[C@H](C)c4cc(F)ccc4F)nc(C)nc3cn2)C1]

Example 75 (80 mg, 190 μmol) was purified by chiral HPLC to give:

Example 101 (32 mg, 38%, e.e. >95%). Rt=3.34 min

Example 102 (32 mg, 38%, e.e. >95%). Rt=4.41 mins

Analytical Method: Instrument: Thermo Fisher UltiMate 3000; Column: YMC Cellulose SB 3μ, 100×4.6; eluent A: methyl tert-butyl ether+0.1 vol % diethylamine; eluent B: ethanol; isocratic: 95% A+5% B; flow: 1.4 ml/min; temperature: 25° C.; UV: 280 nm

Preparative Method: Instrument: PrepCon Labomatic HPLC-3; Column: YMC Cellulose SB 10μ, 250×50; eluent A: methyl tert-butyl ether+0.1 vol % diethylamine; eluent B: ethanol+0.1 vol % diethylamine; isocratic: 95% A+5% B; flow: 80 ml/min; temperature: 25° C.; UV: 280 nm

¹H-NMR (400 MHz, DMSO-d6) δ [ppm]: 1.231 (0.59), 1.570 (4.90), 1.588 (4.94), 1.825 (16.00), 1.931 (0.59), 1.945 (0.61), 1.963 (0.45), 2.184 (0.47), 2.200 (0.56), 2.216 (0.50), 2.305 (14.24), 2.322 (0.63), 2.327 (0.72), 2.332 (0.50), 2.518 (2.73), 2.523 (1.69), 2.665 (0.43), 2.669 (0.61), 2.673 (0.43), 3.306 (0.90), 3.316 (1.26), 3.526 (0.45), 3.532 (0.54), 3.545 (0.61), 3.551 (0.52), 3.565 (0.41), 3.604 (0.43), 3.623 (0.90), 3.641 (0.50), 3.648 (0.63), 3.671 (0.88), 3.687 (0.95), 3.698 (0.81), 3.713 (0.72), 4.395 (0.61), 4.410 (0.61), 5.732 (0.63), 5.750 (0.97), 5.768 (0.63), 7.056 (2.96), 7.094 (0.45), 7.108 (0.50), 7.116 (0.88), 7.126 (0.65), 7.136 (0.63), 7.145 (0.43), 7.216 (0.72), 7.226 (0.77), 7.239 (1.24), 7.250 (1.62), 7.257 (1.17), 7.262 (0.93), 7.266 (0.70), 7.273 (1.11), 7.281 (0.54), 8.194 (1.15), 8.210 (1.15), 8.309 (1.20), 8.328 (1.15), 8.642 (4.36).

### EXAMPLE 102

### N-[(3R)-1-(4-{[(1S)-1-(2,5-difluorophenyl)ethyl]amino}-2-methylpyrido[3,4-d]pyrimidin-6-yl)pyrrolidin-3-yl]acetamide

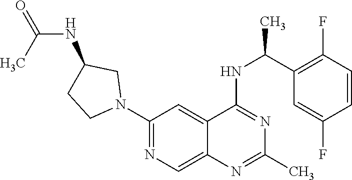 [SMILES: CC(=O)N[C@@H]1CCN(c2cc3c(N[C@@H](C)c4cc(F)ccc4F)nc(C)nc3cn2)C1]

See example 101 for details.

¹H-NMR (400 MHz, DMSO-d6) δ [ppm]: 1.572 (5.06), 1.589 (5.07), 1.830 (16.00), 1.929 (0.63), 1.943 (0.65), 1.960 (0.48), 2.179 (0.53), 2.195 (0.63), 2.210 (0.55), 2.226 (0.44), 2.304 (14.05), 2.518 (0.63), 3.320 (0.92), 3.330 (1.31), 3.552 (0.40), 3.558 (0.63), 3.572 (0.72), 3.578 (0.72), 3.587 (0.72), 3.605 (1.09), 3.623 (0.59), 3.630 (0.54), 3.645 (0.89), 3.660 (0.98), 3.671 (0.84), 3.687 (0.75), 4.387 (0.40), 4.401 (0.66), 4.413 (0.64), 5.726 (0.67), 5.744 (1.02), 5.762 (0.67), 7.049 (3.02), 7.091 (0.48), 7.105 (0.54), 7.113 (0.93), 7.123 (0.69), 7.133 (0.67), 7.143 (0.44), 7.213 (0.76), 7.225 (0.83), 7.231 (0.76), 7.237 (1.37), 7.247 (1.54), 7.254 (1.26), 7.260 (1.00), 7.271 (0.98), 7.277 (0.59), 8.204 (1.24), 8.221 (1.20), 8.308 (1.24), 8.327 (1.18), 8.643 (4.30).

### EXAMPLE 103

### 3-[(1R)-1-({6-[(3R)-3-acetamidopyrrolidin-1-yl]-2-methylpyrido[3,4-d]pyrimidin-4-yl}amino)ethyl]benzoic acid

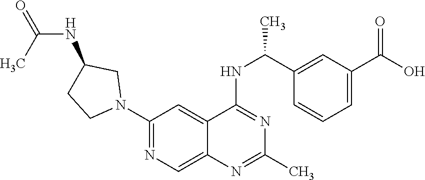 [SMILES: CC(=O)N[C@@H]1CCN(c2cc3c(N[C@H](C)c4cccc(C(=O)O)c4)nc(C)nc3cn2)C1]

To a solution of Example 65 (21.2 mg, 47.3 μmol) in MeOH (2m1) was added 1M NaOH (2 ml). Stirred at RT for 16 h.) Reaction concentrated under reduced pressure and the residue was purified by preparative HPLC (basic method) to give the titled compound (13.8 mg, 64%).

¹H-NMR (400 MHz, DMSO-d6) δ [ppm]: 1.571 (4.77), 1.588 (4.82), 1.815 (16.00), 1.915 (0.63), 1.928 (0.67), 1.946 (0.46), 2.163 (0.51), 2.178 (0.65), 2.194 (0.57), 2.209 (0.44), 2.333 (14.27), 2.522 (0.81), 3.307 (1.33), 3.316 (1.51), 3.334 (1.99), 3.343 (2.31), 3.478 (1.15), 3.490 (1.03), 3.499 (1.08), 3.504 (1.14), 3.516 (1.07), 3.524 (0.94), 3.536 (0.74), 3.576 (0.61), 3.595 (1.11), 3.613 (0.71), 3.620 (0.83), 3.638 (1.17), 3.654 (1.11), 3.666 (0.95), 3.681 (0.84), 4.368 (0.42), 4.381 (0.69), 4.395 (0.69), 5.629 (0.72), 5.647 (1.04), 5.667 (0.70), 7.103 (3.09), 7.203 (1.12), 7.222 (2.51), 7.241 (1.46), 7.363 (1.41), 7.383 (1.14), 7.704 (1.78), 7.722 (1.65), 7.971 (2.63), 8.227 (1.41), 8.244 (1.40), 8.398 (1.29), 8.418 (1.24), 8.608 (4.76).

### EXAMPLE 104

### N-{(3R)-1-[4-({(1R)-1-[3-(hydroxymethyl)phenyl]ethyl}amino)-2-methylpyrido[3,4-d]pyrimidin-6-yl]pyrrolidin-3-yl}acetamide

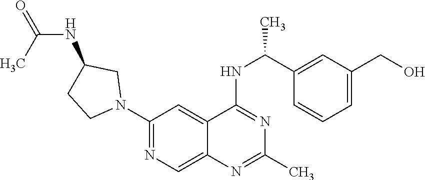 [SMILES: CC(=O)N[C@@H]1CCN(c2cc3c(N[C@H](C)c4cccc(CO)c4)nc(C)nc3cn2)C1]

To a solution of Example 65 (22mg, 49 μmol) in THF (3 ml) and NaBH4 (14.8 mg, 392 μmol) was added and stirred at RT for 1h. To the reaction mixture was added MeOH (3 ml) and stirred at RT for 3 h. The reaction was concentrated and the residue was purified by preparative HPLC (basic method) to give the titled compound (3.4 mg, 16%).

¹H-NMR (400 MHz, DMSO-d6) δ [ppm]: 1.569 (5.26), 1.587 (5.32), 1.821 (16.00), 1.902 (0.45), 1.920 (0.71), 1.933 (0.75), 1.951 (0.56), 2.173 (0.66), 2.189 (0.75), 2.204 (0.68), 2.221 (0.52), 2.334 (14.38), 2.669 (0.41), 3.288 (1.00), 3.298 (1.31), 3.315 (1.92), 3.492 (0.42), 3.517 (0.74), 3.530 (0.78), 3.537 (0.69), 3.550 (0.52), 3.588 (0.48), 3.606 (1.04), 3.624 (0.62), 3.631 (0.73), 3.647 (1.09), 3.662 (1.05), 3.674 (0.90), 3.689 (0.79), 4.374 (0.46), 4.386 (0.78), 4.400 (0.77), 4.471 (5.94), 5.611 (0.77), 5.629 (1.13), 5.647 (0.75), 7.065 (3.32), 7.149 (1.24), 7.167 (1.60), 7.248 (0.92), 7.267 (2.36), 7.286 (3.19), 7.289 (2.65), 7.308 (0.67), 7.402 (2.60), 8.194 (1.37), 8.211 (1.37), 8.316 (1.41), 8.336 (1.39), 8.618 (4.71).

### EXAMPLE 105

### N-[(3R)-1-(4-{[(1R)-1-(3-hydroxyphenyl)ethyl]amino}-2-methylpyrido[3,4-d]pyrimidin-6-yl)pyrrolidin-3-yl]acetamide

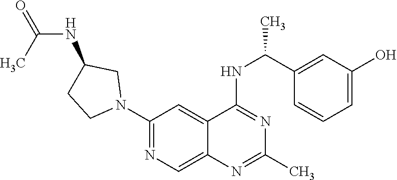 [SMILES: CC(=O)N[C@@H]1CCN(c2cc3c(N[C@H](C)c4cccc(O)c4)nc(C)nc3cn2)C1]

In a vessel flushed with argon was added tBuBrettPhos Pd G3 (8.19 mg, 9.59 μmol), tBuBrettPhos (4.65 mg, 9.59 μmol), Cs2CO3 (43.7 mg, 134 μmol) and Example 62 (45.0 mg, 95.9 μmol). The vessel was flushed again with Argon and toluene (1.2 ml) and 2,2-difluoroethan-1-ol (61 μl, 960 μmol) were added. The reaction mixture was heated at 80° C. for 16 h. The reaction mixture was diluted with EtOAc, washed with water, filtered through a hydrophobic membran concentrated under vacuum. The residue was purified by silica chromatography (DCM:EtOH) to give Example 105 (5 mg, 13%) and Example 106 (10 mg, 22%)

¹H-NMR (400 MHz, DMSO-d6) δ [ppm]: 0.850 (0.66), 0.867 (1.18), 0.872 (0.87), 0.887 (1.18), 0.905 (1.63), 0.924 (0.69), 1.107 (1.28), 1.232 (1.49), 1.256 (0.52), 1.278 (0.76), 1.295 (0.76), 1.316 (0.49), 1.349 (1.28), 1.537 (4.69), 1.555 (4.69), 1.820 (16.00), 1.921 (0.59), 1.934 (0.62), 1.952 (0.42), 2.075 (0.83), 2.172 (0.45), 2.188 (0.56), 2.202 (0.49), 2.318 (0.49), 2.323 (1.08), 2.327 (1.56), 2.332 (1.56), 2.339 (14.30), 2.518 (4.65), 2.523 (3.16), 2.540 (0.52), 2.660 (0.45), 2.665 (0.97), 2.669 (1.35), 2.674 (0.94), 2.679 (0.45), 3.285 (0.76), 3.295 (0.94), 3.505 (0.56), 3.511 (0.62), 3.519 (0.56), 3.532 (0.59), 3.539 (0.52), 3.606 (0.87), 3.623 (0.49), 3.631 (0.59), 3.646 (0.87), 3.661 (0.94), 3.673 (0.80), 3.688 (0.69), 4.249 (0.42), 4.371 (0.42), 4.386 (0.62), 4.398 (0.59), 5.555 (0.62), 5.574 (0.90), 5.592 (0.62), 6.591 (1.01), 6.595 (1.01), 6.597 (0.97), 6.611 (1.04), 6.615 (1.11), 6.813 (2.12), 6.818 (1.49), 6.836 (1.21), 6.855 (1.39), 7.064 (2.88), 7.086 (1.67), 7.105 (2.57), 7.124 (1.25), 8.088 (2.08), 8.185 (1.21), 8.202 (1.18), 8.251 (1.25), 8.271 (1.15), 8.624 (4.23), 9.304 (2.74).

### EXAMPLE 106

### N-{(3R)-1-[4-({(1R)-1-[3-(2,2-difluoroethoxy)phenyl]ethyl}amino)-2-methylpyrido[3,4-d]pyrimidin-6-yl]pyrrolidin-3-yl}acetamide

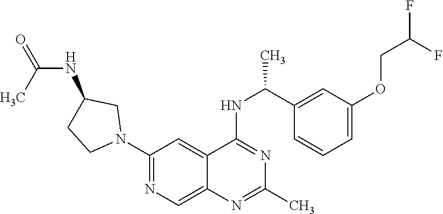 [SMILES: CC(=O)N[C@@H]1CCN(c2cc3c(N[C@H](C)c4cccc(OCC(F)F)c4)nc(C)nc3cn2)C1]

See Example 105 for details.

¹H-NMR (400 MHz, DMSO-d6) δ [ppm]: 1.107 (0.69), 1.563 (4.39), 1.581 (4.42), 1.820 (16.00), 1.922 (0.55), 1.936 (0.57), 2.174 (0.48), 2.191 (0.53), 2.207 (0.46), 2.322 (1.01), 2.333 (13.97), 2.518 (2.95), 2.522 (2.01), 2.664 (0.70), 2.668 (0.95), 2.673 (0.67), 3.294 (0.67), 3.304 (0.83), 3.516 (0.42), 3.521 (0.52), 3.528 (0.42), 3.534 (0.56), 3.541 (0.49), 3.609 (0.84), 3.627 (0.46), 3.634 (0.57), 3.653 (0.94), 3.668 (0.88), 3.679 (0.76), 3.695 (0.67), 4.244 (0.78), 4.252 (0.81), 4.281 (1.59), 4.289 (1.55), 4.317 (0.84), 4.326 (0.74), 4.388 (0.57), 4.402 (0.56), 5.591 (0.60), 5.609 (0.88), 5.628 (0.60), 6.230 (0.69), 6.358 (0.62), 6.366 (1.40), 6.375 (0.64), 6.502 (0.59), 6.859 (0.80), 6.861 (0.83), 6.865 (0.84), 6.868 (0.85), 6.881 (0.95), 6.886 (1.02), 7.052 (5.41), 7.060 (1.59), 7.068 (1.48), 7.247 (1.57), 7.268 (2.60), 7.288 (1.17), 8.186 (1.16), 8.203 (1.13), 8.262 (1.16), 8.282 (1.12), 8.626 (3.97).

### EXAMPLE 107

### N-[(3R)-1-(4-{[(1R)-1-{3-[(E)-2-ethoxyethenyl]phenyl}ethyl]amino}-2-methylpyrido[3,4-d]pyrimidin-6-yl)pyrrolidin-3-yl]acetamide

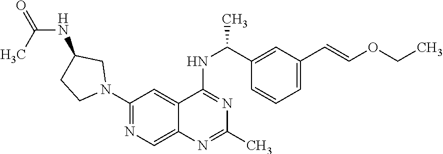 [SMILES: CCO/C=C/c1cccc([C@@H](C)Nc2nc(C)nc3cnc(N4CC[C@@H](NC(C)=O)C4)cc23)c1]

To a solution of Example 62 (600 mg, 1.28 mmol) in dioxane (8.1 ml) was added 2-[(E)-2-ethoxyethenyl]-4,4,5,5-tetramethyl-1,3,2-dioxaborolane (253 mg, 1.28 mmol), followed by K2CO3 (589 mg, 4.26 mmol) and Pd(PPh3)4 (123 mg, 107 μmol) and water (1.62 ml). The reaction was heated at 90° C. for 16 h. The reaction was concentrated and purified by silica chromatography (EtOH:DCM) to give the titled compound (480 mg, 81%).

¹H-NMR (400 MHz, DMSO-d6) δ [ppm]: 1.222 (4.24), 1.239 (9.16), 1.256 (4.35), 1.562 (4.62), 1.579 (4.65), 1.819 (16.00), 1.919 (0.59), 1.931 (0.65), 1.949 (0.46), 2.171 (0.49), 2.187 (0.59), 2.202 (0.54), 2.218 (0.41), 2.323 (1.30), 2.327 (1.95), 2.331 (1.86), 2.341 (13.89), 2.518 (10.73), 2.523 (7.46), 2.665 (1.30), 2.669 (1.76), 2.673 (1.27), 3.286 (0.73), 3.296 (0.76), 3.518 (0.59), 3.531 (0.65), 3.551 (0.43), 3.586 (0.43), 3.605 (0.95), 3.623 (0.54), 3.630 (0.65), 3.647 (1.00), 3.662 (0.97), 3.673 (0.84), 3.689 (0.76), 3.846 (1.16), 3.864 (3.81), 3.881 (3.81), 3.899 (1.16), 4.385 (0.65), 4.400 (0.68), 5.574 (0.70), 5.593 (0.95), 5.611 (0.65), 5.794 (2.24), 5.827 (2.38), 7.055 (2.97), 7.143 (0.76), 7.148 (1.05), 7.153 (1.05), 7.169 (4.08), 7.177 (2.73), 7.188 (1.11), 7.202 (2.89), 7.213 (0.46), 7.314 (2.30), 8.185 (1.30), 8.202 (1.24), 8.261 (1.24), 8.280 (1.19), 8.622 (4.27).

TABLE 4
Examples 108-115
Using the method described for Example 25: Intermediate 11 was treated with the corresponding
phenylethan-1-amines or their hydrochloride salts and gave the desired compounds after preparative
HPLC purification (basic method) and/or optionally silica chromatography.

| | Structure |
|---|---|
| | IUPAC-Name |
| Example | ¹H-NMR |
| 108 | 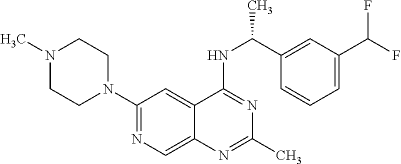 [SMILES: Cc1nc(N[C@H](C)c2cccc(C(F)F)c2)c2cc(N3CCN(C)CC3)ncc2n1] N-{(1R)-1-[3-(difluoromethyl)phenyl]ethyl}-2-methyl-6-(4-methylpiperazin-1- yl)pyrido[3,4-d]pyrimidin-4-amine ¹H-NMR (400 MHz, DMSO-d6) δ [ppm]: 1.602 (5.48), 1.619 (5.55), 2.246 (11.82), 2.322 (0.41), 2.327 (0.56), 2.341 (16.00), 2.456 (2.69), 2.469 (4.06), 2.482 (3.41), 2.522 (1.81), 3.533 (2.60), 3.546 (3.48), 3.557 (2.63), 5.622 (0.72), 5.639 (1.10), 5.658 (0.74), 6.889 (1.36), 7.029 (2.77), 7.168 (1.23), 7.409 (3.24), 7.420 (0.96), 7.440 (1.73), 7.463 (1.28), 7.482 (1.87), 7.500 (0.80), 7.600 (1.31), 7.619 (1.06), 7.636 (2.20), 8.395 (1.29), 8.414 (1.26), 8.655 (4.80). |
| 109 | 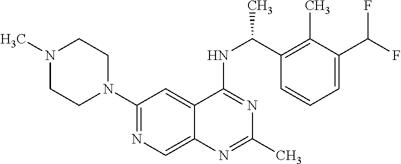 [SMILES: Cc1nc(N[C@H](C)c2cccc(C(F)F)c2C)c2cc(N3CCN(C)CC3)ncc2n1] N-{(1R)-1-[3-(difluoromethyl)-2-methylphenyl]ethyl}-2-methyl-6-(4-methylpiperazin-1- yl)pyrido[3,4-d]pyrimidin-4-amine ¹H-NMR (400 MHz, DMSO-d6) δ [ppm]: 1.540 (4.76), 1.558 (4.78), 2.250 (11.08), 2.318 (16.00), 2.461 (2.38), 2.473 (3.62), 2.518 (1.21), 2.523 (1.02), 2.535 (7.48), 3.536 (2.29), 3.549 (3.02), 3.560 (2.27), 5.714 (0.70), 5.732 (1.07), 5.749 (0.69), 7.078 (0.89), 7.214 (1.92), 7.280 (0.66), 7.298 (1.60), 7.318 (1.06), 7.352 (0.78), 7.381 (1.57), 7.399 (1.04), 7.426 (2.92), 7.625 (1.25), 7.644 (1.11), 8.466 (1.17), 8.484 (1.13), 8.636 (4.58). |
| 110 | 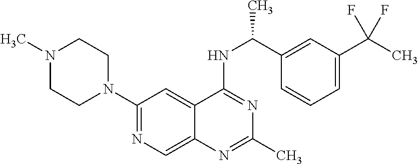 [SMILES: Cc1nc(N[C@H](C)c2cccc(C(C)(F)F)c2)c2cc(N3CCN(C)CC3)ncc2n1] N-{(1R)-1-[3-(1,1-difluoroethyl)phenyl]ethyl}-2-methyl-6-(4-methylpiperazin-1- yl)pyrido[3,4-d]pyrimidin-4-amine ¹H-NMR (400 MHz, DMSO-d6) δ [ppm]: 1.604 (5.30), 1.622 (5.32), 1.907 (5.01), 1.955 (9.83), 2.002 (4.36), 2.246 (11.68), 2.327 (0.52), 2.332 (0.44), 2.348 (16.00), 2.456 (2.52), 2.468 (3.77), 2.481 (3.12), 2.518 (1.66), 2.523 (1.13), 2.669 (0.48), 3.532 (2.42), 3.545 (3.16), 3.556 (2.37), 5.613 (0.71), 5.631 (1.06), 5.649 (0.71), 7.401 (3.10), 7.430 (3.33), 7.449 (1.82), 7.468 (0.59), 7.549 (1.20), 7.566 (1.02), 7.647 (2.21), 8.381 (1.27), 8.400 (1.23), 8.653 (4.78). |
| 111 | 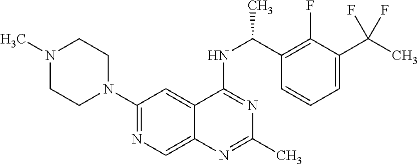 [SMILES: Cc1nc(N[C@H](C)c2cccc(C(C)(F)F)c2F)c2cc(N3CCN(C)CC3)ncc2n1] N-{(1R)-1-[3-(1,1-difluoroethyl)-2-fluorophenyl]ethyl}-2-methyl-6-(4-methylpiperazin-1- yl)pyrido[3,4-d]pyrimidin-4-amine ¹H-NMR (400 MHz, DMSO-d6) δ [ppm]: 1.599 (5.19), 1.617 (5.26), 1.981 (2.85), 2.029 (5.48), 2.077 (2.52), 2.253 (11.78), 2.301 (16.00), 2.322 (0.58), 2.326 (0.67), 2.331 (0.49), 2.466 (2.80), 2.478 (4.20), 2.518 (3.74), 2.522 (2.61), 2.669 (0.51), 3.545 (2.55), 3.558 (3.40), 3.570 (2.58), 5.744 (0.79), 5.762 (1.23), 5.779 (0.79), 7.231 (0.89), 7.250 (1.94), 7.269 (1.13), 7.416 (0.74), 7.439 (3.53), 7.450 (0.77), 7.586 (0.62), 7.604 (1.13), 7.620 (0.59), 8.429 (1.31), 8.446 (1.25), 8.656 (4.69). |
| 112 | 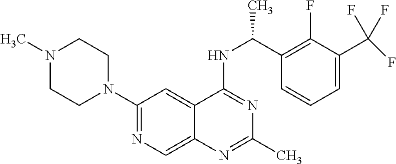 [SMILES: Cc1nc(N[C@H](C)c2cccc(C(F)(F)F)c2F)c2cc(N3CCN(C)CC3)ncc2n1] N-{(1R)-1-[2-fluoro-3-(trifluoromethyl)phenyl]ethyl}-2-methyl-6-(4-methylpiperazin-1- yl)pyrido[3,4-d]pyrimidin-4-amine ¹H-NMR (400 MHz, DMSO-d6) δ [ppm]: 1.622 (4.95), 1.640 (5.02), 2.253 (11.21), 2.283 (16.00), 2.466 (2.49), 2.478 (3.90), 2.518 (3.01), 2.522 (2.06), 3.547 (2.39), 3.559 (3.18), 3.570 (2.41), 5.708 (0.77), 5.726 (1.18), 5.744 (0.75), 7.339 (0.71), 7.358 (1.55), 7.377 (0.89), 7.434 (2.96), 7.628 (0.64), 7.645 (1.13), 7.662 (0.56), 7.757 (0.59), 7.774 (1.09), 7.792 (0.55), 8.474 (1.21), 8.491 (1.19), 8.658 (4.42). |
| 113 | 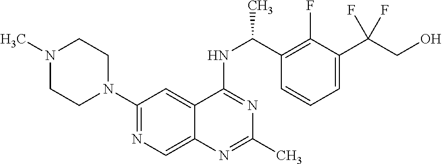 [SMILES: Cc1nc(N[C@H](C)c2cccc(C(F)(F)CO)c2F)c2cc(N3CCN(C)CC3)ncc2n1] 2,2-difluoro-2-{2-fluoro-3-[(1R)-1-{[2-methyl-6-(4-methylpiperazin-1-yl)pyrido[3,4- d]pyrimidin-4-yl]amino}ethyl]phenyl}ethan-1-ol ¹H-NMR (400 MHz, DMSO-d6) δ [ppm]: 1.230 (0.82), 1.594 (5.15), 1.612 (5.15), 1.711 (0.42), 1.720 (0.42), 1.728 (1.08), 1.735 (0.41), 1.744 (0.43), 2.253 (11.89), 2.309 (16.00), 2.322 (0.87), 2.327 (0.93), 2.332 (0.68), 2.465 (3.01), 2.478 (4.93), 2.518 (2.99), 2.523 (1.92), 2.665 (0.57), 2.669 (0.82), 2.673 (0.57), 2.998 (0.42), 3.005 (0.78), 3.014 (0.78), 3.021 (0.40), 3.546 (2.69), 3.559 (3.53), 3.570 (2.59), 3.898 (0.78), 3.935 (1.47), 3.970 (0.72), 5.736 (1.00), 5.755 (0.97), 5.772 (1.29), 5.790 (0.81), 7.237 (0.93), 7.256 (2.04), 7.275 (1.20), 7.399 (0.76), 7.417 (1.24), 7.443 (3.33), 7.603 (0.67), 7.619 (1.17), 7.637 (0.60), 8.432 (1.36), 8.451 (1.29), 8.655 (5.09). |
| 114 | 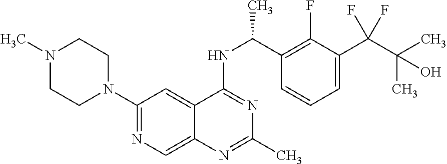 [SMILES: Cc1nc(N[C@H](C)c2cccc(C(F)(F)C(C)(C)O)c2F)c2cc(N3CCN(C)CC3)ncc2n1] 1,1-difluoro-1-{2-fluoro-3-[(1R)-1-{[2-methyl-6-(4-methylpiperazin-1-yl)pyrido[3,4- d]pyrimidin-4-yl]amino}ethyl]phenyl}-2-methylpropan-2-ol ¹H-NMR (400 MHz, DMSO-d6) δ [ppm]: 1.202 (5.89), 1.230 (6.28), 1.580 (4.59), 1.597 (4.58), 2.254 (11.17), 2.290 (16.00), 2.327 (0.54), 2.467 (2.51), 2.480 (3.97), 2.518 (1.79), 2.523 (1.25), 2.669 (0.53), 3.547 (2.34), 3.560 (3.08), 3.572 (2.29), 5.336 (7.38), 5.749 (0.73), 5.767 (1.11), 5.785 (0.71), 7.197 (0.71), 7.216 (1.68), 7.235 (1.10), 7.291 (0.64), 7.295 (0.74), 7.312 (1.04), 7.328 (0.48), 7.446 (2.93), 7.553 (0.58), 7.569 (1.00), 7.585 (0.53), 8.420 (1.23), 8.439 (1.19), 8.654 (4.71). |
| 115 | 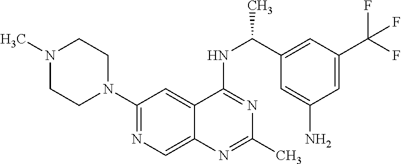 [SMILES: Cc1nc(N[C@H](C)c2cc(N)cc(C(F)(F)F)c2)c2cc(N3CCN(C)CC3)ncc2n1] N-{(1R)-1-[3-amino-5-(trifluoromethyl)phenyl]ethyl}-2-methyl-6-(4-methylpiperazin-1- yl)pyrido[3,4-d]pyrimidin-4-amine ¹H-NMR (400 MHz, DMSO-d6) δ [ppm]: 1.546 (4.72), 1.563 (4.69), 2.246 (11.13), 2.356 (16.00), 2.456 (2.55), 2.468 (3.72), 2.481 (3.42), 2.518 (2.94), 2.523 (2.10), 3.532 (2.34), 3.545 (3.10), 3.556 (2.26), 5.512 (0.67), 5.530 (1.02), 5.549 (0.90), 5.565 (3.65), 6.699 (2.21), 6.831 (2.02), 6.860 (2.24), 7.412 (2.90), 8.323 (1.25), 8.342 (1.20), 8.658 (4.66). |

TABLE 5
Examples 116-122
Using the method described for Example 25: Intermediate 12 was treated with the corresponding
phenylethan-1-amines or their hydrochloride salts and gave the desired compounds after preparative
HPLC purification (basic method) and/or optionally silica chromatography.

| | Structure |
|---|---|
| | IUPAC-Name |
| Example | ¹H-NMR |
| 116 | 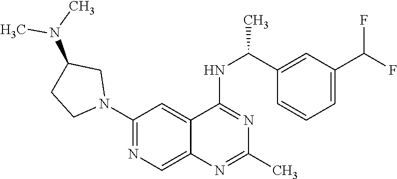 [SMILES: Cc1nc(N[C@H](C)c2cccc(C(F)F)c2)c2cc(N3CC[C@@H](N(C)C)C3)ncc2n1] N-{(1R)-1-[3-(difluoromethyl)phenyl]ethyl}-6-[(3R)-3-(dimethylamino)pyrrolidin-1-yl]-2- methylpyrido[3,4-d]pyrimidin-4-amine ¹H-NMR (400 MHz, DMSO-d6) δ [ppm]: 1.610 (3.39), 1.628 (3.42), 1.866 (0.40), 2.232 (16.00), 2.325 (10.45), 2.518 (1.44), 2.523 (1.03), 3.162 (0.56), 3.182 (0.62), 3.186 (0.69), 3.207 (0.55), 3.391 (0.50), 3.408 (0.52), 3.657 (0.56), 3.730 (0.47), 3.747 (0.55), 3.754 (0.53), 3.773 (0.42), 5.632 (0.46), 5.651 (0.67), 5.669 (0.44), 6.886 (0.86), 7.018 (2.06), 7.025 (1.94), 7.165 (0.78), 7.415 (0.49), 7.434 (1.04), 7.459 (0.77), 7.478 (1.15), 7.497 (0.49), 7.601 (0.80), 7.621 (0.65), 7.640 (1.33), 8.309 (0.82), 8.328 (0.80), 8.624 (2.84). |
| 117 | 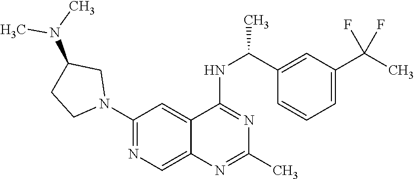 [SMILES: Cc1nc(N[C@H](C)c2cccc(C(C)(F)F)c2)c2cc(N3CC[C@@H](N(C)C)C3)ncc2n1] N-{(1R)-1-[3-(1,1-difluoroethyl)phenyl]ethyl}-6-[(3R)-3-(dimethylamino)pyrrolidin-1-yl]- 2-methylpyrido[3,4-d]pyrimidin-4-amine ¹H-NMR (400 MHz, DMSO-d6) δ [ppm]: 1.611 (3.14), 1.629 (3.15), 1.904 (3.01), 1.952 (6.25), 1.998 (2.67), 2.231 (16.00), 2.332 (10.49), 2.518 (0.75), 2.523 (0.50), 3.161 (0.45), 3.181 (0.48), 3.186 (0.56), 3.206 (0.44), 3.390 (0.41), 3.407 (0.46), 3.653 (0.47), 3.726 (0.42), 3.744 (0.44), 3.751 (0.45), 5.622 (0.42), 5.640 (0.62), 5.658 (0.41), 7.009 (2.00), 7.425 (1.98), 7.445 (1.14), 7.552 (0.75), 7.568 (0.61), 7.654 (1.41), 8.297 (0.82), 8.316 (0.79), 8.623 (2.89). |
| 118 | 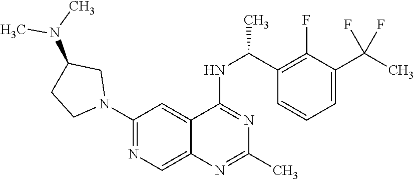 [SMILES: Cc1nc(N[C@H](C)c2cccc(C(C)(F)F)c2F)c2cc(N3CC[C@@H](N(C)C)C3)ncc2n1] N-{(1R)-1-[3-(1,1-difluoroethyl)-2-fluorophenyl]ethyl}-6-[(3R)-3- (dimethylamino)pyrrolidin-1-yl]-2-methylpyrido[3,4-d]pyrimidin-4-amine ¹H-NMR (400 MHz, DMSO-d6) δ [ppm]: 1.605 (3.22), 1.623 (3.27), 1.874 (0.41), 1.981 (1.81), 2.029 (3.48), 2.076 (1.59), 2.214 (0.44), 2.239 (16.00), 2.285 (10.27), 2.518 (1.32), 2.523 (0.91), 2.844 (0.40), 3.176 (0.57), 3.197 (0.63), 3.201 (0.70), 3.221 (0.55), 3.402 (0.52), 3.419 (0.51), 3.668 (0.58), 3.743 (0.49), 3.761 (0.56), 3.768 (0.54), 3.786 (0.44), 5.757 (0.49), 5.775 (0.77), 5.793 (0.49), 7.048 (2.03), 7.225 (0.56), 7.244 (1.24), 7.263 (0.71), 7.410 (0.44), 7.428 (0.75), 7.607 (0.71), 8.340 (0.82), 8.358 (0.78), 8.624 (2.89). |
| 119 | 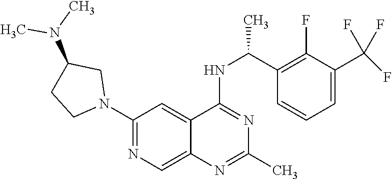 [SMILES: Cc1nc(N[C@H](C)c2cccc(C(F)(F)F)c2F)c2cc(N3CC[C@@H](N(C)C)C3)ncc2n1] 6-[(3R)-3-(dimethylamino)pyrrolidin-1-yl]-N-{(1R)-1-[2-fluoro-3- (trifluoromethyl)phenyl]ethyl}-2-methylpyrido[3,4-d]pyrimidin-4-amine ¹H-NMR (400 MHz, DMSO-d6) δ [ppm]: 1.627 (3.29), 1.645 (3.31), 1.875 (0.41), 2.215 (0.44), 2.239 (16.00), 2.266 (9.85), 2.518 (1.60), 2.523 (1.11), 2.845 (0.41), 3.177 (0.57), 3.198 (0.63), 3.202 (0.70), 3.223 (0.56), 3.403 (0.51), 3.420 (0.52), 3.668 (0.58), 3.742 (0.47), 3.759 (0.56), 3.766 (0.54), 3.785 (0.44), 5.722 (0.51), 5.739 (0.78), 5.757 (0.50), 7.041 (2.04), 7.334 (0.48), 7.353 (1.04), 7.373 (0.60), 7.622 (0.43), 7.640 (0.75), 7.776 (0.73), 8.387 (0.82), 8.404 (0.80), 8.627 (2.85). |
| 120 | 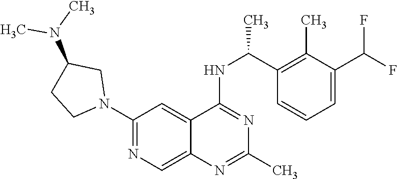 [SMILES: Cc1nc(N[C@H](C)c2cccc(C(F)F)c2C)c2cc(N3CC[C@@H](N(C)C)C3)ncc2n1] N-{(1R)-1-[3-(difluoromethyl)-2-methylphenyl]ethyl}-6-[(3R)-3- (dimethylamino)pyrrolidin-1-yl]-2-methylpyrido[3,4-d]pyrimidin-4-amine ¹H-NMR (400 MHz, DMSO-d6) δ [ppm]: 1.546 (3.20), 1.563 (3.22), 1.871 (0.40), 2.237 (16.00), 2.300 (10.34), 2.518 (1.09), 2.523 (0.82), 2.539 (4.98), 3.170 (0.57), 3.190 (0.65), 3.195 (0.71), 3.215 (0.61), 3.394 (0.51), 3.412 (0.50), 3.660 (0.56), 3.738 (0.47), 3.755 (0.55), 3.762 (0.53), 3.780 (0.42), 5.723 (0.47), 5.741 (0.73), 5.758 (0.47), 7.039 (2.03), 7.075 (0.62), 7.212 (1.30), 1.Til. (0.45), 7.291 (1.09), 7.310 (0.72), 7.350 (0.53), 7.374 (1.07), 7.392 (0.69), 7.637 (0.85), 7.656 (0.75), 8.381 (0.81), 8.400 (0.78), 8.604 (2.96). |
| 121 | 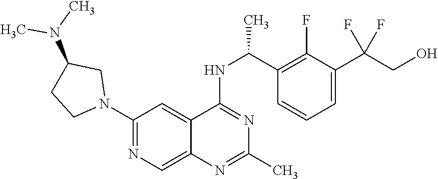 [SMILES: Cc1nc(N[C@H](C)c2cccc(C(F)(F)CO)c2F)c2cc(N3CC[C@@H](N(C)C)C3)ncc2n1] 2-{3-[(1R)-1-({6-[(3R)-3-(dimethylamino)pyrrolidin-1-yl]-2-methylpyrido[3,4-d]pyrimidin- 4-yl}amino)ethyl]-2-fluorophenyl}-2,2-difluoroethan-1-ol ¹H-NMR (400 MHz, DMSO-d6) δ [ppm]: 1.600 (3.25), 1.617 (3.24), 1.874 (0.41), 2.216 (0.44), 2.239 (16.00), 2.293 (10.17), 2.518 (1.36), 2.523 (0.86), 2.846 (0.41), 3.176 (0.58), 3.197 (0.63), 3.201 (0.71), 3.222 (0.56), 3.403 (0.55), 3.420 (0.52), 3.670 (0.57), 3.744 (0.48), 3.762 (0.56), 3.769 (0.55), 3.787 (0.43), 3.928 (0.59), 3.941 (0.63), 5.725 (0.68), 5.767 (0.51), 5.786 (0.77), 5.803 (0.50), 7.048 (2.04), 7.231 (0.59), 7.250 (1.30), 7.270 (0.77), 7.394 (0.46), 7.412 (0.74), 7.622 (0.71), 8.333 (0.83), 8.352 (0.80), 8.626 (3.07). |
| 122 | 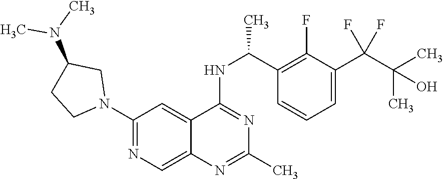 [SMILES: Cc1nc(N[C@H](C)c2cccc(C(F)(F)C(C)(C)O)c2F)c2cc(N3CC[C@@H](N(C)C)C3)ncc2n1] 1-{3-[(1R)-1-({6-[(3R)-3-(dimethylamino)pyrrolidin-1-yl]-2-methylpyrido[3,4-d]pyrimidin- 4-yl}amino)ethyl]-2-fluorophenyl}-1,1-difluoro-2-methylpropan-2-ol ¹H-NMR (400 MHz, DMSO-d6) δ [ppm]: 1.202 (4.13), 1.231 (4.46), 1.585 (3.14), 1.603 (3.16), 1.877 (0.41), 2.220 (0.47), 2.241 (16.00), 2.273 (10.47), 2.327 (0.53), 2.518 (2.92), 2.523 (2.07), 2.669 (0.52), 2.848 (0.42), 3.178 (0.56), 3.203 (0.69), 3.224 (0.55), 3.405 (0.52), 3.423 (0.52), 3.671 (0.59), 3.745 (0.48), 3.763 (0.55), 3.770 (0.55), 3.787 (0.41), 5.335 (4.47), 5.762 (0.48), 5.779 (0.73), 5.798 (0.48), 7.055 (2.09), 7.191 (0.48), 7.210 (1.16), 7.229 (0.76), 7.289 (0.52), 7.307 (0.74), 7.555 (0.41), 7.572 (0.72), 8.333 (0.84), 8.351 (0.82), 8.623 (2.94). |

TABLE 6
Examples 123-129
Using the method described for Example 25: Intermediate 13 was treated with the corresponding
phenylethan-1-amines or their hydrochloride salts and gave the desired compounds after preparative
HPLC purification (basic method) and/or optionally silica chromatography.

| | Structure |
|---|---|
| | IUPAC-Name |
| Example | ¹H-NMR |
| 123 | 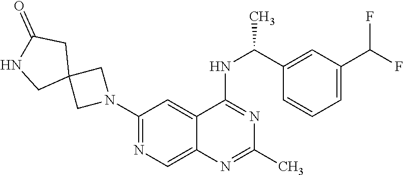 [SMILES: Cc1nc(N[C@H](C)c2cccc(C(F)F)c2)c2cc(N3CC4(CNC(=O)C4)C3)ncc2n1] 2-[4-({(1R)-1-[3-(difluoromethyl)phenyl]ethyl}amino)-2-methylpyrido[3,4-d]pyrimidin-6- yl]-2,6-diazaspiro[3.4]octan-7-one ¹H-NMR (400 MHz, DMSO-d6) δ [ppm]: 1.230 (0.46), 1.593 (5.56), 1.611 (5.62), 1.983 (0.69), 2.128 (0.62), 2.336 (16.00), 2.357 (0.91), 2.518 (3.80), 2.522 (2.76), 2.546 (8.53), 2.664 (0.42), 2.668 (0.55), 3.036 (1.02), 3.390 (0.66), 3.526 (6.95), 3.966 (0.80), 3.990 (8.71), 4.015 (0.89), 5.609 (0.75), 5.627 (1.11), 5.645 (0.74), 6.885 (1.39), 7.024 (2.88), 7.115 (3.61), 7.164 (1.44), 7.266 (0.59), 7.393 (0.54), 7.415 (1.02), 7.436 (1.84), 7.458 (1.39), 7.477 (1.98), 7.496 (0.83), 7.595 (1.42), 7.615 (1.15), 7.636 (2.33), 7.679 (2.36), 8.388 (1.30), 8.408 (1.28), 8.625 (4.66). |
| 124 | 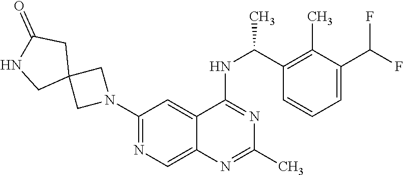 [SMILES: Cc1nc(N[C@H](C)c2cccc(C(F)F)c2C)c2cc(N3CC4(CNC(=O)C4)C3)ncc2n1] 2-[4-({(1R)-1-[3-(difluoromethyl)-2-methylphenyl]ethyl}amino)-2-methylpyrido[3,4- d]pyrimidin-6-yl]-2,6-diazaspiro[3.4]octan-7-one ¹H-NMR (400 MHz, DMSO-d6) δ [ppm]: 1.532 (4.91), 1.550 (4.97), 2.126 (0.43), 2.313 (16.00), 2.327 (0.72), 2.332 (0.47), 2.518 (2.07), 2.523 (1.92), 2.532 (8.13), 2.550 (8.04), 2.669 (0.49), 3.530 (6.48), 3.971 (0.89), 3.996 (7.69), 4.021 (0.85), 5.706 (0.74), 5.723 (1.14), 5.741 (0.72), 7.074 (0.96), 7.136 (3.44), 7.212 (2.06), 7.274 (0.73), 7.293 (1.77), 7.312 (1.18), 7.349 (0.85), 7.378 (1.77), 7.396 (1.15), 7.628 (1.40), 7.647 (1.22), 7.682 (2.26), 8.456 (1.25), 8.475 (1.20), 8.606 (4.60). |
| 125 | 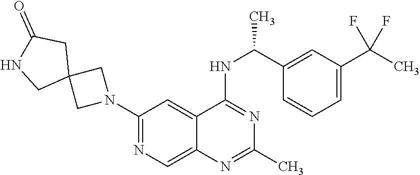 [SMILES: Cc1nc(N[C@H](C)c2cccc(C(C)(F)F)c2)c2cc(N3CC4(CNC(=O)C4)C3)ncc2n1] 2-[4-({(1R)-1-[3-(1,1-difluoroethyl)phenyl]ethyl}amino)-2-methylpyrido[3,4-d]pyrimidin- 6-yl]-2,6-diazaspiro[3.4]octan-7-one ¹H-NMR (400 MHz, DMSO-d6) δ [ppm]: 1.595 (5.67), 1.613 (5.63), 1.906 (5.29), 1.953 (10.41), 1.962 (0.75), 2.000 (4.56), 2.133 (1.13), 2.318 (0.52), 2.322 (0.69), 2.327 (0.89), 2.332 (0.89), 2.343 (16.00), 2.518 (3.25), 2.523 (2.16), 2.546 (8.58), 2.665 (0.51), 2.669 (0.71), 2.673 (0.50), 3.526 (7.10), 3.965 (0.84), 3.989 (9.09), 4.013 (0.81), 5.599 (0.78), 5.618 (1.18), 5.636 (0.77), 7.107 (3.71), 7.407 (0.52), 7.412 (0.59), 7.426 (4.20), 7.444 (2.07), 7.464 (0.77), 7.546 (1.44), 7.563 (1.21), 7.648 (2.54), 7.677 (2.42), 8.375 (1.41), 8.394 (1.35), 8.623 (4.83). |
| 126 | 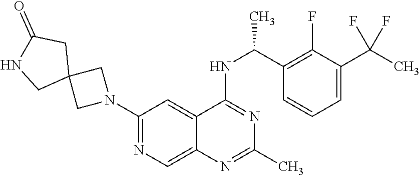 [SMILES: Cc1nc(N[C@H](C)c2cccc(C(C)(F)F)c2F)c2cc(N3CC4(CNC(=O)C4)C3)ncc2n1] 2-[4-({(1R)-1-[3-(1,1-difluoroethyl)-2-fluorophenyl]ethyl}amino)-2-methylpyrido[3,4- d]pyrimidin-6-yl]-2,6-diazaspiro[3.4]octan-7-one ¹H-NMR (400 MHz, DMSO-d6) δ [ppm]: 1.591 (5.18), 1.608 (5.20), 1.979 (3.35), 1.999 (0.42), 2.027 (5.80), 2.075 (2.62), 2.096 (0.88), 2.298 (16.00), 2.322 (0.43), 2.327 (0.54), 2.518 (2.15), 2.523 (1.62), 2.553 (8.03), 2.669 (0.52), 3.347 (0.50), 3.533 (6.75), 3.979 (0.81), 4.003 (8.30), 4.027 (0.83), 5.738 (0.78), 5.756 (1.21), 5.774 (0.76), 7.143 (3.45), 7.228 (0.89), 7.247 (2.05), 7.266 (1.23), 7.414 (0.76), 7.432 (1.21), 7.448 (0.58), 7.585 (0.64), 7.603 (1.15), 7.620 (0.60), 7.686 (2.29), 8.421 (1.27), 8.440 (1.23), 8.627 (4.65). |
| 127 | 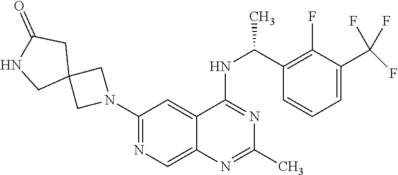 [SMILES: Cc1nc(N[C@H](C)c2cccc(C(F)(F)F)c2F)c2cc(N3CC4(CNC(=O)C4)C3)ncc2n1] 2-[4-({(1R)-1-[2-fluoro-3-(trifluoromethyl)phenyl]ethyl}amino)-2-methylpyrido[3,4- d]pyrimidin-6-yl]-2,6-diazaspiro[3.4]octan-7-one ¹H-NMR (400 MHz, DMSO-d6) δ [ppm]: 1.613 (5.10), 1.631 (5.10), 1.941 (0.73), 2.083 (0.83), 2.280 (16.00), 2.322 (0.49), 2.327 (0.63), 2.332 (0.45), 2.518 (2.48), 2.523 (1.66), 2.555 (7.86), 2.664 (0.44), 2.669 (0.64), 2.673 (0.45), 3.533 (6.64), 3.981 (0.77), 4.005 (8.44), 4.028 (0.72), 5.704 (0.80), 5.722 (1.21), 5.739 (0.78), 7.133 (3.37), 7.337 (0.78), 7.357 (1.64), 7.376 (0.91), 7.627 (0.74), 7.644 (1.21), 7.661 (0.61), 7.686 (2.17), 7.756 (0.67), 7.774 (1.13), 7.790 (0.57), 8.470 (1.24), 8.488 (1.18), 8.630 (4.52), 8.632 (4.40). |
| 128 | 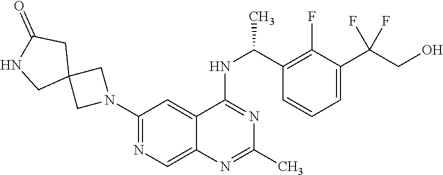 [SMILES: Cc1nc(N[C@H](C)c2cccc(C(F)(F)CO)c2F)c2cc(N3CC4(CNC(=O)C4)C3)ncc2n1] 2-[4-({(1R)-1-[3-(1,1-difluoro-2-hydroxyethyl)-2-fluorophenyl]ethyl}amino)-2- methylpyrido[3,4-d]pyrimidin-6-yl]-2,6-diazaspiro[3.4]octan-7-one ¹H-NMR (400 MHz, DMSO-d6) δ [ppm]: 1.586 (5.00), 1.603 (5.02), 2.306 (16.00), 2.322 (0.49), 2.327 (0.55), 2.518 (1.69), 2.523 (1.15), 2.553 (7.91), 2.669 (0.48), 3.533 (6.66), 3.889 (0.53), 3.905 (0.61), 3.925 (1.06), 3.941 (1.14), 3.961 (0.58), 3.979 (1.31), 4.003 (8.10), 4.028 (0.81), 5.704 (0.87), 5.720 (1.94), 5.736 (0.93), 5.748 (0.85), 5.767 (1.23), 5.784 (0.78), 7.142 (3.49), 7.234 (0.93), 7.254 (2.05), 7.273 (1.21), 7.398 (0.74), 7.416 (1.18), 7.431 (0.57), 7.598 (0.63), 7.615 (1.13), 7.632 (0.60), 7.684 (2.48), 8.141 (0.80), 8.409 (1.30), 8.428 (1.25), 8.628 (4.72). |
| 129 | 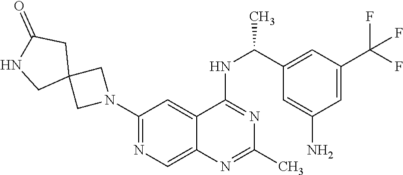 [SMILES: Cc1nc(N[C@H](C)c2cc(N)cc(C(F)(F)F)c2)c2cc(N3CC4(CNC(=O)C4)C3)ncc2n1] 2-[4-({(1R)-1-[3-amino-5-(trifluoromethyl)phenyl]ethyl}amino)-2-methylpyrido[3,4- d]pyrimidin-6-yl]-2,6-diazaspiro[3.4]octan-7-one ¹H-NMR (400 MHz, DMSO-d6) δ [ppm]: −0.129 (0.41), 0.733 (0.60), 0.749 (0.64), 0.756 (0.67), 0.819 (0.64), 0.821 (0.41), 0.837 (1.34), 0.840 (0.81), 0.856 (0.65), 0.944 (0.41), 0.971 (1.17), 0.989 (2.09), 1.006 (1.43), 1.094 (0.41), 1.167 (0.74), 1.473 (4.90), 1.491 (4.87), 1.688 (0.57), 2.001 (1.59), 2.258 (0.48), 2.263 (0.62), 2.267 (0.50), 2.288 (16.00), 2.453 (2.24), 2.459 (1.60), 2.481 (7.74), 2.605 (0.53), 3.358 (0.52), 3.371 (0.52), 3.376 (0.50), 3.388 (0.52), 3.460 (6.55), 3.897 (0.80), 3.921 (8.44), 3.945 (0.75), 4.292 (0.65), 5.435 (0.73), 5.453 (1.07), 5.472 (0.85), 5.495 (3.72), 5.694 (0.58), 6.634 (2.39), 6.764 (2.19), 6.798 (2.40), 7.054 (3.41), 7.614 (2.42), 8.244 (1.27), 8.264 (1.21), 8.563 (4.67), 8.565 (4.58). |

TABLE 7
Examples 130-136
Using the method described for Example 25: Intermediate 14 was treated with the corresponding
phenylethan-1-amines or their hydrochloride salts and gave the desired compounds after preparative
HPLC purification (basic method) and/or optionally silica chromatography.

| | Structure |
|---|---|
| | IUPAC-Name |
| Example | ¹H-NMR |
| 130 | 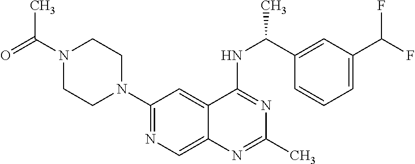 [SMILES: CC(=O)N1CCN(c2cc3c(N[C@H](C)c4cccc(C(F)F)c4)nc(C)nc3cn2)CC1] 1-{4-[4-({(1R)-1-[3-(difluoromethyl)phenyl]ethyl}amino)-2-methylpyrido[3,4-d]pyrimidin- 6-yl]piperazin-1-yl}ethan-1-one ¹H-NMR (400 MHz, DMSO-d6) δ [ppm]: 1.609 (5.25), 1.627 (5.25), 2.071 (16.00), 2.327 (0.47), 2.331 (0.42), 2.348 (15.78), 2.518 (2.46), 2.523 (1.72), 2.669 (0.40), 3.334 (15.26), 3.528 (1.45), 3.539 (1.39), 5.628 (0.67), 5.646 (1.03), 5.664 (0.68), 6.889 (1.30), 7.029 (2.70), 7.169 (1.21), 7.424 (0.77), 7.443 (1.72), 7.458 (3.14), 7.465 (1.54), 7.485 (1.82), 7.504 (0.77), 7.603 (1.26), 7.623 (1.02), 7.640 (2.12), 8.427 (1.17), 8.446 (1.15), 8.682 (4.60). |
| 131 | 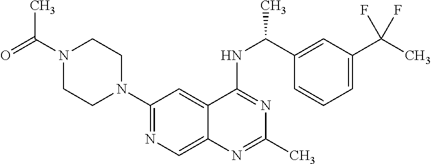 [SMILES: CC(=O)N1CCN(c2cc3c(N[C@H](C)c4cccc(C(C)(F)F)c4)nc(C)nc3cn2)CC1] 1-{4-[4-({(1R)-1-[3-(1,1-difluoroethyl)phenyl]ethyl}amino)-2-methylpyrido[3,4- d]pyrimidin-6-yl]piperazin-1-yl}ethan-1-one ¹H-NMR (400 MHz, DMSO-d6) δ [ppm]: 1.610 (4.90), 1.628 (4.90), 1.908 (4.94), 1.956 (9.64), 2.003 (4.16), 2.071 (16.00), 2.355 (15.42), 2.518 (1.59), 2.523 (1.12), 3.523 (1.30), 3.527 (1.28), 3.537 (1.24), 3.613 (5.98), 5.618 (0.64), 5.637 (0.97), 5.655 (0.64), 7.433 (3.34), 7.450 (4.22), 7.471 (0.58), 7.553 (1.12), 7.570 (0.96), 7.650 (2.06), 8.413 (1.14), 8.432 (1.09), 8.679 (4.36). |
| 132 | 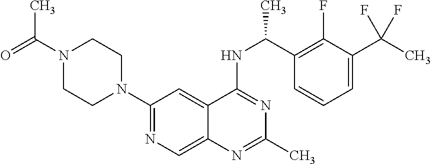 [SMILES: CC(=O)N1CCN(c2cc3c(N[C@H](C)c4cccc(C(C)(F)F)c4F)nc(C)nc3cn2)CC1] 1-{4-[4-({(1R)-1-[3-(1,1-difluoroethyl)-2-fluorophenyl]ethyl}amino)-2-methylpyrido[3,4- d]pyrimidin-6-yl]piperazin-1-yl}ethan-1-one ¹H-NMR (400 MHz, DMSO-d6) δ [ppm]: 1.606 (4.51), 1.624 (4.49), 1.955 (0.52), 1.982 (2.54), 2.030 (4.88), 2.077 (16.00), 2.308 (13.61), 2.322 (0.49), 2.327 (0.46), 2.518 (1.33), 2.523 (0.81), 3.537 (1.29), 3.552 (1.24), 3.622 (4.28), 3.627 (4.27), 5.749 (0.64), 5.767 (0.98), 5.785 (0.63), 7.233 (0.79), 7.252 (1.72), 7.271 (1.01), 7.419 (0.62), 7.438 (1.02), 7.453 (0.50), 7.487 (2.67), 7.592 (0.55), 7.610 (0.96), 7.626 (0.48), 8.463 (0.99), 8.481 (0.93), 8.682 (4.20). |
| 133 | 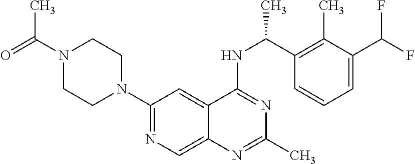 [SMILES: CC(=O)N1CCN(c2cc3c(N[C@H](C)c4cccc(C(F)F)c4C)nc(C)nc3cn2)CC1] 1-{4-[4-({(1R)-1-[3-(difluoromethyl)-2-methylphenyl]ethyl}amino)-2-methylpyrido[3,4- d]pyrimidin-6-yl]piperazin-1-yl}ethan-1-one ¹H-NMR (400 MHz, DMSO-d6) δ [ppm]: 1.548 (4.85), 1.565 (4.77), 2.075 (15.84), 2.325 (16.00), 2.518 (1.72), 2.523 (1.44), 2.534 (7.70), 2.669 (0.48), 3.530 (1.36), 3.617 (6.49), 5.720 (0.68), 5.737 (1.05), 5.755 (0.68), 7.079 (0.92), 7.216 (1.98), 7.282 (0.69), 7.301 (1.65), 7.321 (1.10), 7.354 (0.82), 7.385 (1.63), 7.403 (1.06), 7.473 (2.95), 7.628 (1.28), 7.646 (1.15), 8.491 (1.11), 8.509 (1.05), 8.663 (4.68). |
| 134 | 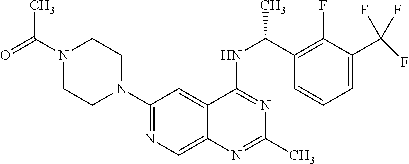 [SMILES: CC(=O)N1CCN(c2cc3c(N[C@H](C)c4cccc(C(F)(F)F)c4F)nc(C)nc3cn2)CC1] 1-{4-[4-({(1R)-1-[2-fluoro-3-(trifluoromethyl)phenyl]ethyl}amino)-2-methylpyrido[3,4- d]pyrimidin-6-yl]piperazin-1-yl}ethan-1-one ¹H-NMR (400 MHz, DMSO-d6) δ [ppm]: 1.629 (4.78), 1.647 (4.76), 2.019 (0.63), 2.078 (16.00), 2.105 (0.58), 2.185 (0.72), 2.290 (15.63), 2.518 (1.84), 2.523 (1.23), 3.539 (1.41), 3.553 (1.41), 3.623 (4.35), 3.629 (4.49), 5.714 (0.72), 5.731 (1.09), 5.749 (0.71), 7.341 (0.79), 7.361 (1.52), 7.381 (0.90), 7.479 (2.84), 7.632 (0.65), 7.649 (1.11), 7.669 (0.56), 7.762 (0.56), 7.778 (1.03), 7.796 (0.52), 8.504 (1.10), 8.521 (1.08), 8.685 (4.45). |
| 135 | 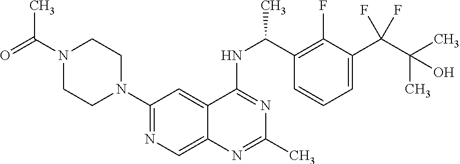 [SMILES: CC(=O)N1CCN(c2cc3c(N[C@H](C)c4cccc(C(F)(F)C(C)(C)O)c4F)nc(C)nc3cn2)CC1] 1-{4-[4-({(1R)-1-[3-(1,1-difluoro-2-hydroxy-2-methylpropyl)-2- fluorophenyl]ethyl}amino)-2-methylpyrido[3,4-d]pyrimidin-6-yl]piperazin-1-yl}ethan-1- one ¹H-NMR (400 MHz, DMSO-d6) δ [ppm]: 1.202 (6.01), 1.230 (6.32), 1.587 (4.57), 1.605 (4.56), 2.078 (16.00), 2.297 (15.31), 2.327 (0.48), 2.518 (1.78), 2.523 (1.12), 2.669 (0.47), 3.540 (1.42), 3.553 (1.36), 3.623 (4.61), 3.628 (4.67), 5.339 (3.65), 5.755 (0.71), 5.773 (1.09), 5.791 (0.71), 7.199 (0.71), 7.218 (1.67), 7.237 (1.11), 7.299 (0.76), 7.316 (1.05), 7.331 (0.51), 7.336 (0.46), 7.492 (2.87), 7.556 (0.59), 7.573 (1.01), 7.589 (0.53), 8.447 (1.19), 8.466 (1.15), 8.682 (4.68). |
| 136 | 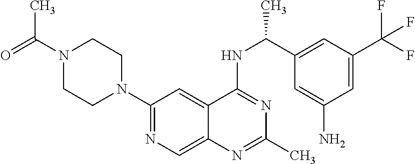 [SMILES: CC(=O)N1CCN(c2cc3c(N[C@H](C)c4cc(N)cc(C(F)(F)F)c4)nc(C)nc3cn2)CC1] 1-{4-[4-({(1R)-1-[3-amino-5-(trifluoromethyl)phenyl]ethyl}amino)-2-methylpyrido[3,4- d]pyrimidin-6-yl]piperazin-1-yl}ethan-1-one ¹H-NMR (400 MHz, DMSO-d6) δ [ppm]: 1.035 (0.83), 1.052 (1.57), 1.070 (0.84), 1.231 (0.59), 1.552 (3.96), 1.570 (3.98), 1.956 (1.50), 2.071 (11.11), 2.327 (0.66), 2.363 (10.67), 2.669 (0.58), 3.330 (16.00), 3.435 (0.40), 3.440 (0.40), 3.527 (1.49), 5.518 (0.63), 5.536 (1.00), 5.567 (3.47), 6.703 (2.05), 6.836 (1.96), 6.863 (2.15), 7.456 (2.54), 8.345 (1.12), 8.365 (1.09), 8.684 (3.73). |

TABLE 8
Examples 137-257
Using the method described for Example 3: Example 2 was treated with nitrogen containing
nucleophile at 130° C. The desired compounds were obtainied after preparative HPLC purification
(basic method) and/or optionally silica chromatography.

| | Structure |
|---|---|
| | IUPAC-Name |
| Example | ¹H-NMR |
| 137 | 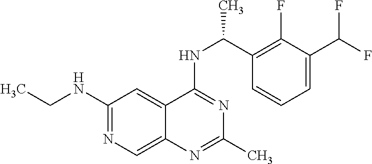 [SMILES: CCNc1cc2c(N[C@H](C)c3cccc(C(F)F)c3F)nc(C)nc2cn1] N⁴-{(1R)-1-[3-(difluoromethyl)-2-fluorophenyl]ethyl}-N⁶-ethyl-2-methylpyrido[3,4- d]pyrimidine-4,6-diamine ¹H-NMR (400 MHz, DMSO-d6) δ [ppm]: 1.205 (4.27), 1.222 (9.34), 1.241 (4.52), 1.593 (5.53), 1.610 (5.70), 2.277 (16.00), 2.323 (0.75), 2.327 (1.01), 2.331 (0.80), 2.665 (0.71), 2.669 (1.01), 2.673 (0.80), 3.233 (0.59), 3.250 (2.01), 3.264 (2.30), 3.268 (2.39), 3.282 (2.14), 3.300 (0.92), 5.742 (0.84), 5.760 (1.34), 5.778 (0.88), 6.443 (0.88), 6.457 (1.80), 6.471 (0.92), 7.010 (3.77), 7.099 (1.21), 7.235 (2.51), 7.270 (0.92), 7.289 (2.05), 7.308 (1.21), 7.370 (1.09), 7.479 (0.75), 7.496 (1.26), 7.514 (0.67), 7.641 (0.67), 7.659 (1.26), 7.678 (0.67), 8.324 (1.42), 8.342 (1.42), 8.536 (4.69). |
| 138 | 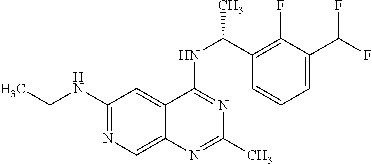 [SMILES: CCNc1cc2c(N[C@H](C)c3cccc(C(F)F)c3F)nc(C)nc2cn1] N⁶-cyclopropyl-N⁴-{(1R)-1-[3-(difluoromethyl)-2-fluorophenyl]ethyl}-2-methylpyrido[3,4- d]pyrimidine-4,6-diamine ¹H-NMR (500 MHz, DMSO-d6) δ [ppm]: 0.466 (0.42), 0.479 (0.74), 0.482 (0.81), 0.487 (0.81), 0.529 (0.75), 0.533 (0.82), 0.536 (0.81), 0.540 (0.77), 0.552 (0.48), 0.557 (0.43), 0.801 (0.70), 0.806 (1.88), 0.810 (1.60), 0.814 (1.01), 0.819 (1.88), 0.823 (1.54), 0.829 (0.64), 1.107 (6.96), 1.614 (5.27), 1.628 (5.27), 2.285 (16.00), 2.514 (1.90), 2.518 (1.71), 2.522 (1.37), 2.535 (0.46), 2.542 (0.73), 2.549 (0.92), 2.555 (0.92), 2.562 (0.66), 4.189 (0.69), 5.769 (0.79), 5.784 (1.21), 5.798 (0.80), 6.886 (1.82), 6.892 (1.78), 7.131 (1.06), 7.202 (3.68), 7.240 (2.23), 7.280 (0.88), 7.296 (1.88), 7.311 (1.07), 7.349 (0.93), 7.484 (0.59), 7.497 (1.03), 7.512 (0.53), 7.660 (0.57), 7.674 (1.04), 7.688 (0.52), 8.385 (1.31), 8.400 (1.28), 8.540 (4.21). |
| 139 | 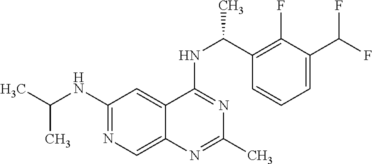 [SMILES: Cc1nc(N[C@H](C)c2cccc(C(F)F)c2F)c2cc(NC(C)C)ncc2n1] N⁴-{(1R)-1-[3-(difluoromethyl)-2-fluorophenyl]ethyl}-2-methyl-N⁶-(propan-2- yl)pyrido[3,4-d]pyrimidine-4,6-diamine ¹H-NMR (400 MHz, DMSO-d6) δ [ppm]: 0.905 (0.54), 1.040 (0.80), 1.057 (0.80), 1.154 (1.27), 1.172 (2.50), 1.190 (1.76), 1.198 (7.27), 1.213 (11.14), 1.227 (7.76), 1.593 (5.33), 1.610 (5.43), 1.988 (4.10), 2.273 (16.00), 2.323 (0.73), 2.327 (0.99), 2.331 (0.75), 2.665 (0.76), 2.669 (1.04), 2.673 (0.80), 2.994 (1.25), 3.367 (0.55), 3.877 (0.50), 3.893 (0.73), 3.898 (0.62), 3.909 (0.60), 3.914 (0.73), 3.930 (0.52), 4.017 (0.93), 4.035 (0.91), 5.737 (0.83), 5.755 (1.32), 5.759 (1.41), 5.772 (0.85), 6.266 (1.67), 6.287 (1.63), 7.020 (3.58), 7.100 (1.19), 7.236 (2.41),7.270 (0.89), 7.289 (1.98), 7.308 (1.17), 7.371 (1.06), 7.479 (0.72), 7.497 (1.20), 7.513 (0.62), 7.641 (0.65), 7.659 (1.20), 7.677 (0.62), 8.089 (0.86), 8.290 (1.38), 8.308 (1.37), 8.530 (4.54). |
| 140 | 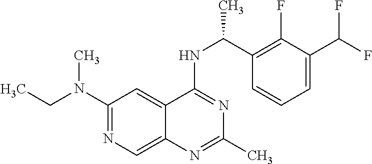 [SMILES: CCN(C)c1cc2c(N[C@H](C)c3cccc(C(F)F)c3F)nc(C)nc2cn1] N⁴-{(1R)-1-[3-(difluoromethyl)-2-fluorophenyl]ethyl}-N⁶-ethyl-N⁶,2-dimethylpyrido[3,4- d]pyrimidine-4,6-diamine ¹H-NMR (400 MHz, DMSO-d6) δ [ppm]: 0.000 (1.51), 0.869 (0.38), 0.907 (0.63), 1.087 (2.90), 1.104 (7.06), 1.111 (3.02), 1.122 (3.02), 1.612 (4.91), 1.630 (4.91), 2.087 (0.38), 2.287 (16.00), 2.466 (0.88), 2.523 (3.53), 2.527 (2.39), 3.075 (14.87), 3.312 (0.63), 3.315 (0.76), 3.320 (0.76), 3.326 (0.88), 3.390 (1.13), 3.400 (0.63), 3.410 (0.38), 3.633 (0.38), 3.650 (0.76), 3.667 (1.13), 3.685 (1.13), 3.699 (1.13), 3.716 (1.13), 3.734 (0.63), 5.763 (0.76), 5.781 (1.13), 5.799 (0.76), 7.104 (1.13), 7.177 (3.15), 7.240 (2.39), 7.277 (0.76), 7.296 (1.76), 7.315 (1.01), 7.375 (1.01), 7.486 (0.63), 7.502 (1.01), 7.520 (0.50), 7.640 (0.50), 7.658 (1.01), 7.676 (0.50), 8.091 (1.26), 8.393 (1.13), 8.411 (1.13), 8.629 (4.03). |
| 141 | 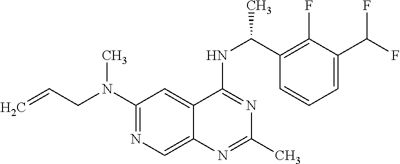 [SMILES: C=CCN(C)c1cc2c(N[C@H](C)c3cccc(C(F)F)c3F)nc(C)nc2cn1] N⁴-{(1R)-1-[3-(difluoromethyl)-2-fluorophenyl]ethyl}-N⁶,2-dimethyl-N⁶-(prop-2-en-1- yl)pyrido[3,4-d]pyrimidine-4,6-diamine LC-MS ( ): Rₜ = min; MS ( ): m/z = ¹H-NMR (400 MHz, DMSO-d6) δ [ppm]: 1.608 (5.16), 1.625 (5.14), 2.290 (16.00), 2.322 (0.68), 2.326 (0.84), 2.331 (0.61), 2.518 (3.87), 2.522 (2.52), 2.665 (0.57), 2.668 (0.81), 2.673 (0.57), 3.065 (14.82), 4.267 (1.07), 4.282 (1.85), 4.296 (1.13), 5.131 (2.14), 5.133 (2.17), 5.152 (1.13), 5.156 (1.48), 5.176 (1.41), 5.180 (1.18), 5.759 (0.78), 5.777 (1.21), 5.795 (0.78), 5.814 (0.48), 5.828 (0.82), 5.841 (0.59), 5.854 (0.85), 5.871 (0.79), 5.884 (0.50), 5.897 (0.68), 7.103 (1.17), 7.230 (3.50), 7.239 (2.63), 7.275 (0.85), 7.294 (1.88), 7.313 (1.09), 7.375 (1.01), 7.485 (0.65), 7.502 (1.10), 7.520 (0.54), 7.636 (0.61), 7.653 (1.09), 7.672 (0.56), 8.394 (1.26), 8.412 (1.23), 8.630 (4.38). |
| 142 | 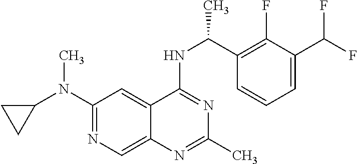 [SMILES: Cc1nc(N[C@H](C)c2cccc(C(F)F)c2F)c2cc(N(C)C3CC3)ncc2n1] N⁶-cyclopropyl-N⁴-{(1R)-1-[3-(difluoromethyl)-2-fluorophenyl]ethyl}-N⁶,2- dimethylpyrido[3,4-d]pyrimidine-4,6-diamine ¹H-NMR (400 MHz, DMSO-d6) δ [ppm]: 0.569 (0.46), 0.587 (0.76), 0.597 (0.78), 0.606 (0.44), 0.623 (0.42), 0.632 (0.78), 0.643 (0.81), 0.653 (0.51), 0.965 (0.55), 0.979 (2.19), 0.995 (2.19), 1.008 (0.53), 1.619 (5.24), 1.636 (5.24), 2.299 (15.19), 2.322 (1.04), 2.327 (1.39), 2.522 (4.13), 2.612 (0.60), 2.620 (0.78), 2.628 (1.11), 2.637 (0.76), 2.644 (0.58), 2.665 (1.02), 2.669 (1.39), 3.147 (16.00), 5.780 (0.81), 5.798 (1.22), 5.816 (0.78), 7.104 (1.13), 7.240 (2.33), 7.274 (0.85), 7.293 (1.87), 7.312 (1.11), 7.376 (1.04), 7.473 (3.67), 7.499 (1.15), 7.516 (0.55), 7.645 (0.62), 7.662 (1.11), 7.681 (0.58), 8.444 (1.32), 8.463 (1.27), 8.666 (4.34). |
| 143 | 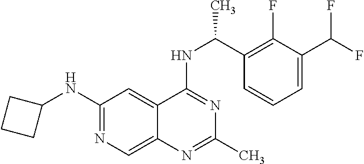 [SMILES: Cc1nc(N[C@H](C)c2cccc(C(F)F)c2F)c2cc(NC3CCC3)ncc2n1] N⁶-cyclobutyl-N⁴-{(1R)-1-[3-(difluoromethyl)-2-fluorophenyl]ethyl}-2-methylpyrido[3,4- d]pyrimidine-4,6-diamine ¹H-NMR (400 MHz, DMSO-d6) δ [ppm]: 0.000 (0.75), 0.869 (0.68), 0.888 (0.61), 0.907 (1.13), 0.926 (0.52), 1.605 (5.17), 1.623 (5.14), 1.678 (0.61), 1.685 (0.61), 1.697 (0.54), 1.704 (1.27), 1.711 (0.57), 1.728 (0.97), 1.748 (0.82), 1.922 (0.45), 1.928 (0.48), 1.944 (0.57), 1.950 (0.66), 1.959 (0.63), 1.966 (0.66), 1.977 (0.54), 1.982 (0.61), 1.987 (0.63), 2.013 (0.41), 2.276 (16.00), 2.382 (0.48), 2.395 (0.82), 2.400 (1.11), 2.411 (0.82), 2.422 (1.02), 2.429 (0.75), 2.441 (0.45), 2.466 (0.43), 2.521 (4.17), 2.525 (2.81), 4.130 (0.41), 4.151 (0.77), 4.170 (0.77), 4.190 (0.41), 5.751 (0.79), 5.769 (1.20), 5.787 (0.75), 6.797 (1.61), 6.816 (1.56), 6.946 (3.31), 7.102 (1.18), 7.238 (2.45), 7.276 (0.84), 7.295 (1.86), 7.315 (1.07), 7.373 (1.02), 7.482 (0.63), 7.499 (1.09), 7.517 (0.57), 7.646 (0.57), 7.663 (1.04), 7.681 (0.52), 8.091 (2.04), 8.313 (1.25), 8.332 (1.20), 8.529 (4.33). |
| 144 | 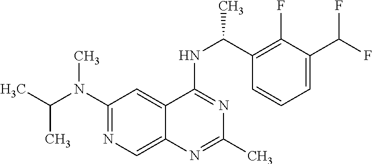 [SMILES: Cc1nc(N[C@H](C)c2cccc(C(F)F)c2F)c2cc(N(C)C(C)C)ncc2n1] N⁴-{(1R)-1-[3-(difluoromethyl)-2-fluorophenyl]ethyl}-N⁶,2-dimethyl-N⁶-(propan-2- yl)pyrido[3,4-d]pyrimidine-4,6-diamine ¹H-NMR (500 MHz, DMSO-d6) δ [ppm]: 1.107 (6.45), 1.128 (1.32), 1.145 (7.40), 1.157 (13.49), 1.170 (7.20), 1.232 (0.44), 1.610 (6.24), 1.624 (6.18), 2.285 (16.00), 2.310 (1.64), 2.514 (5.12), 2.518 (4.23), 2.522 (3.32), 2.549 (1.39), 2.880 (1.55), 2.899 (15.48), 4.191 (0.52), 4.980 (0.95), 4.994 (1.26), 5.007 (0.94), 5.766 (0.92), 5.781 (1.41), 5.795 (0.93), 7.129 (1.26), 7.175 (3.70), 7.238 (2.55), 7.277 (1.05), 7.293 (2.22), 7.308 (1.28), 7.346 (1.09), 7.485 (0.74), 7.499 (1.28), 7.513 (0.67), 7.642 (0.67), 7.656 (1.21), 7.671 (0.60), 8.088 (0.41), 8.393 (1.40), 8.408 (1.37), 8.628 (4.68). |
| 145 | 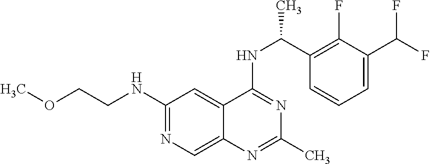 [SMILES: COCCNc1cc2c(N[C@H](C)c3cccc(C(F)F)c3F)nc(C)nc2cn1] N⁴-{(1R)-1-[3-(difluoromethyl)-2-fluorophenyl]ethyl}-N⁶-(2-methoxyethyl)-2- methylpyrido[3,4-d]pyrimidine-4,6-diamine ¹H-NMR (400 MHz, DMSO-d6) δ [ppm]: 1.591 (3.01), 1.609 (3.01), 2.278 (9.27), 2.518 (4.47), 2.522 (2.80), 3.299 (16.00), 3.435 (0.97), 3.449 (1.41), 3.464 (0.73), 3.532 (1.57), 3.546 (2.47), 3.563 (0.73), 5.734 (0.45), 5.752 (0.70), 5.770 (0.45), 6.386 (0.45), 6.401 (0.96), 6.415 (0.43), 7.081 (1.98), 7.099 (0.70), 7.235 (1.41), 7.270 (0.50), 7.289 (1.10), 7.307 (0.63), 7.371 (0.59), 7.496 (0.63), 7.659 (0.63), 8.088 (0.50), 8.324 (0.75), 8.343 (0.71), 8.541 (2.52). |
| 146 | 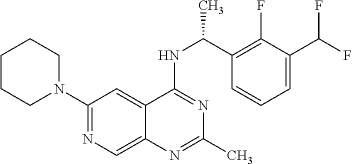 [SMILES: Cc1nc(N[C@H](C)c2cccc(C(F)F)c2F)c2cc(N3CCCCC3)ncc2n1] N-{(1R)-1-[3-(difluoromethyl)-2-fluorophenyl]ethyl}-2-methyl-6-(piperidin-1- yl)pyrido[3,4-d]pyrimidin-4-amine ¹H-NMR (400 MHz, DMSO-d6) δ [ppm]: 1.107 (8.31), 1.605 (5.67), 1.623 (8.51), 1.631 (7.32), 2.294 (16.00), 2.518 (3.69), 2.523 (2.60), 2.539 (0.82), 3.590 (3.77), 4.192 (0.69), 5.752 (0.76), 5.770 (1.19), 5.788 (0.76), 7.103 (1.13), 7.238 (2.36), 7.276 (0.85), 7.295 (1.85), 7.315 (1.07), 7.374 (1.02), 7.403 (3.12), 7.485 (0.64), 7.502 (1.10), 7.520 (0.54), 7.633 (0.59), 7.650 (1.09), 7.669 (0.55), 8.408 (1.26), 8.427 (1.22), 8.637 (4.54). |
| 147 | 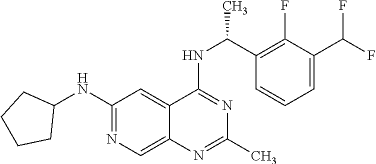 [SMILES: Cc1nc(N[C@H](C)c2cccc(C(F)F)c2F)c2cc(NC3CCCC3)ncc2n1] N⁶-cyclopentyl-N⁴-{(1R)-1-[3-(difluoromethyl)-2-fluorophenyl]ethyl}-2-methylpyrido[3,4- d]pyrimidine-4,6-diamine ¹H-NMR (500 MHz, DMSO-d6) δ [ppm]: 1.493 (0.65), 1.505 (0.83), 1.518 (0.88), 1.530 (0.74), 1.542 (0.59), 1.553 (0.51), 1.561 (0.67), 1.577 (1.00), 1.588 (1.13), 1.599 (6.09), 1.613 (5.48), 1.708 (0.62), 1.719 (1.01), 1.729 (1.04), 1.739 (0.83), 2.015 (0.86), 2.027 (0.90), 2.272 (16.00), 2.514 (2.18), 2.518 (1.73), 2.522 (1.32), 4.002 (0.44), 4.015 (0.82), 4.029 (0.83), 4.043 (0.46), 5.748 (0.81), 5.763 (1.26), 5.777 (0.80), 6.477 (1.72), 6.492 (1.64), 6.993 (3.57), 7.127 (1.10), 7.235 (2.26), 7.275 (0.91), 7.290 (1.94), 7.305 (1.08), 7.344 (0.97), 7.482 (0.63), 7.495 (1.08), 7.509 (0.55), 7.646 (0.61), 7.660 (1.09), 7.675 (0.55), 8.291 (1.35), 8.306 (1.31), 8.526 (4.33). |
| 148 | 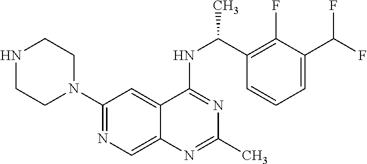 [SMILES: Cc1nc(N[C@H](C)c2cccc(C(F)F)c2F)c2cc(N3CCNCC3)ncc2n1] N-{(1R)-1-[3-(difluoromethyl)-2-fluorophenyl]ethyl}-2-methyl-6-(piperazin-1- yl)pyrido[3,4-d]pyrimidin-4-amine ¹H-NMR (500 MHz, DMSO-d6) δ [ppm]: 1.734 (4.33), 1.749 (4.27), 2.514 (1.89), 2.518 (1.74), 2.522 (1.46), 2.538 (8.36), 3.250 (2.65), 3.564 (16.00), 3.925 (2.45), 3.936 (3.23), 3.945 (2.33), 5.975 (0.61), 5.988 (0.89), 6.002 (0.59), 7.132 (1.02), 7.241 (2.08), 7.339 (0.84), 7.354 (1.88), 7.370 (0.99), 7.552 (0.60), 7.566 (1.02), 7.580 (0.52), 7.929 (0.47), 7.943 (0.86), 7.958 (0.46), 8.292 (0.68), 8.833 (3.96), 9.213 (1.05). |
| 149 | 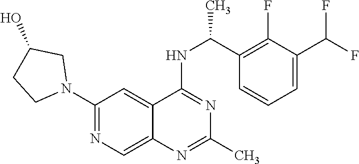 [SMILES: Cc1nc(N[C@H](C)c2cccc(C(F)F)c2F)c2cc(N3CC[C@H](O)C3)ncc2n1] (3S)-1-[4-({(1R)-1-[3-(difluoromethyl)-2-fluorophenyl]ethyl}amino)-2-methylpyrido[3,4- d]pyrimidin-6-yl]pyrrolidin-3-ol ¹H-NMR (400 MHz, DMSO-d6) δ [ppm]: 1.607 (5.24), 1.625 (5.24), 1.954 (0.51), 1.962 (0.54), 1.970 (0.44), 2.068 (0.58), 2.074 (1.14), 2.079 (0.75), 2.089 (0.46), 2.100 (0.61), 2.285 (16.00), 2.322 (0.51), 2.327 (0.68), 2.332 (0.49), 2.518 (2.82), 2.523 (1.73), 2.539 (1.29), 2.664 (0.46), 2.669 (0.68), 2.673 (0.49), 3.401 (0.75), 3.429 (1.00), 3.523 (0.73), 3.550 (1.70), 3.562 (2.29), 3.577 (1.27), 3.589 (1.00), 4.451 (0.85), 5.007 (3.14), 5.016 (3.04), 5.761 (0.80), 5.779 (1.22), 5.797 (0.78), 7.051 (3.26), 7.101 (1.22), 7.237 (2.48), 7.271 (0.88), 7.290 (1.95), 7.309 (1.10), 7.373 (1.05), 7.481 (0.66), 7.498 (1.12), 7.516 (0.54), 7.633 (0.61), 7.651 (1.10), 7.669 (0.54), 8.361 (1.32), 8.379 (1.27), 8.621 (4.77). |
| 150 | 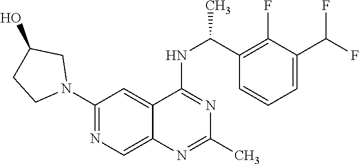 [SMILES: Cc1nc(N[C@H](C)c2cccc(C(F)F)c2F)c2cc(N3CC[C@@H](O)C3)ncc2n1] (3R)-1-[4-({(1R)-1-[3-(difluoromethyl)-2-fluorophenyl]ethyl}amino)-2-methylpyrido[3,4- d]pyrimidin-6-yl]pyrrolidin-3-ol ¹H-NMR (400 MHz, DMSO-d6) δ [ppm]: 1.607 (2.65), 1.624 (2.63), 2.286 (8.09), 2.518 (1.56), 2.523 (1.03), 2.540 (16.00), 3.410 (0.50), 3.530 (0.72), 3.551 (0.83), 3.568 (0.94), 3.580 (0.62), 3.595 (0.46), 3.607 (0.41), 4.447 (0.42), 5.001 (1.46), 5.010 (1.43), 5.779 (0.62), 7.053 (1.66), 7.102 (0.62), 7.238 (1.27), 7.271 (0.44), 7.290 (0.98), 7.309 (0.57), 7.373 (0.53), 7.499 (0.56), 7.652 (0.55), 8.359 (0.66), 8.377 (0.65), 8.621 (2.39). |
| 151 | 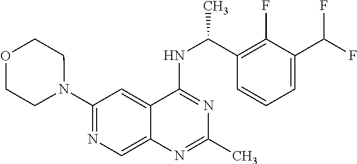 [SMILES: Cc1nc(N[C@H](C)c2cccc(C(F)F)c2F)c2cc(N3CCOCC3)ncc2n1] N-{(1R)-1-[3-(difluoromethyl)-2-fluorophenyl]ethyl}-2-methyl-6-(morpholin-4- yl)pyrido[3,4-d]pyrimidin-4-amine ¹H-NMR (400 MHz, DMSO-d6) δ [ppm]: 1.107 (1.07), 1.607 (5.37), 1.625 (5.38), 2.312 (16.00), 2.327 (0.86), 2.331 (0.64), 2.518 (3.64), 2.523 (2.52), 2.665 (0.44), 2.669 (0.61), 2.673 (0.43), 3.509 (2.88), 3.520 (4.30), 3.533 (3.66), 3.773 (3.62), 3.786 (4.54), 3.797 (3.13), 5.756 (0.83), 5.774 (1.26), 5.791 (0.82), 7.103 (1.18), 7.239 (2.46), 7.278 (0.89), 7.297 (1.97), 7.316 (1.13), 7.375 (1.05), 7.457 (3.34), 7.489 (0.71), 7.507 (1.20), 7.524 (0.59), 7.633 (0.64), 7.652 (1.17), 7.669 (0.59), 8.447 (1.34), 8.465 (1.29), 8.684 (4.78). |
| 152 | 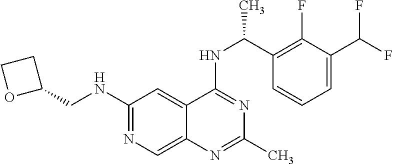 [SMILES: Cc1nc(N[C@H](C)c2cccc(C(F)F)c2F)c2cc(NC[C@H]3CCO3)ncc2n1] N⁴-{(1R)-1-[3-(difluoromethyl)-2-fluorophenyl]ethyl}-2-methyl-N⁶-{[(2RS)-oxetan-2- yl]methyl}pyrido[3,4-d]pyrimidine-4,6-diamine ¹H-NMR (400 MHz, DMSO-d6) δ [ppm]: 1.593 (5.57), 1.610 (5.60), 2.278 (16.00), 2.314 (0.75), 2.322 (1.01), 2.326 (1.35), 2.332 (0.99), 2.437 (0.48), 2.464 (1.11), 2.518 (6.23), 2.522 (3.84), 2.633 (0.67), 2.649 (0.70), 2.654 (0.72), 2.660 (0.96), 2.664 (1.28), 2.669 (1.78), 2.673 (1.37), 3.488 (0.49), 3.504 (0.54), 3.523 (1.16), 3.538 (1.23), 3.544 (0.62), 3.553 (0.60), 3.559 (0.98), 3.578 (0.91), 3.593 (0.66), 4.444 (0.43), 4.447 (0.44), 4.462 (0.96), 4.466 (0.56), 4.470 (0.56), 4.474 (0.76), 4.477 (0.77), 4.482 (0.96), 4.496 (0.59), 4.516 (0.67), 4.537 (1.17), 4.551 (0.84), 4.570 (0.43), 4.914 (0.79), 4.931 (1.10), 4.947 (0.75), 5.734 (0.74), 5.752 (1.11), 5.770 (0.72), 6.566 (0.85), 6.581 (1.71), 6.596 (0.80), 7.099 (1.36), 7.114 (3.71), 7.235 (2.62), 7.270 (0.94), 7.290 (2.03), 7.309 (1.21), 7.371 (1.12), 7.480 (0.75), 7.497 (1.26), 7.515 (0.66), 7.643 (0.68), 7.660 (1.21), 7.679 (0.60), 8.088 (0.51), 8.342 (1.39), 8.361 (1.32), 8.538 (4.69). |
| 153 | 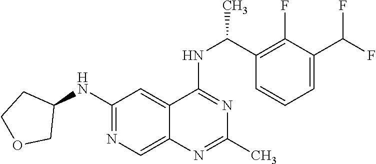 [SMILES: Cc1nc(N[C@H](C)c2cccc(C(F)F)c2F)c2cc(N[C@@H]3CCOC3)ncc2n1] N⁴-{(1R)-1-[3-(difluoromethyl)-2-fluorophenyl]ethyl}-2-methyl-N⁶-[(3R)-oxolan-3- yl]pyrido[3,4-d]pyrimidine-4,6-diamine ¹H-NMR (400 MHz, DMSO-d6) δ [ppm]: 1.597 (5.19), 1.615 (5.31), 1.625 (1.09), 1.643 (0.75), 1.842 (0.42), 1.860 (0.75), 1.874 (0.79), 1.886 (0.49), 1.891 (0.56), 2.241 (0.79), 2.253 (0.45), 2.259 (0.87), 2.273 (1.09), 2.282 (16.00), 2.309 (0.42), 2.518 (4.79), 2.523 (3.31), 2.539 (3.28), 2.914 (1.77), 3.520 (1.22), 3.532 (1.30), 3.542 (1.32), 3.554 (1.34), 3.754 (0.58), 3.769 (0.69), 3.775 (1.36), 3.789 (1.39), 3.795 (1.05), 3.809 (0.85), 3.838 (0.79), 3.856 (1.51), 3.875 (1.14), 3.895 (0.50), 4.022 (1.25), 4.038 (1.60), 4.044 (1.35), 4.060 (1.32), 4.281 (0.69), 4.296 (0.66), 5.741 (0.87), 5.760 (1.23), 5.777 (0.78), 6.747 (1.64), 6.765 (1.59), 7.067 (3.36), 7.101 (1.19), 7.236 (2.46), 7.274 (0.89), 7.293 (1.93), 7.312 (1.13), 7.372 (1.03), 7.482 (0.68), 7.499 (1.19), 7.517 (0.68), 7.644 (0.64), 7.662 (1.14), 7.680 (0.60), 8.333 (1.30), 8.351 (1.26), 8.560 (4.37), 8.788 (0.53). |
| 154 | 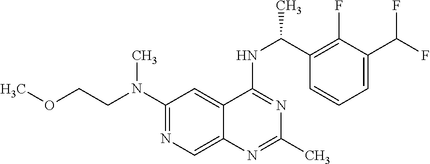 [SMILES: COCCN(C)c1cc2c(N[C@H](C)c3cccc(C(F)F)c3F)nc(C)nc2cn1] N⁴-{(1R)-1-[3-(difluoromethyl)-2-fluorophenyl]ethyl}-N⁶-(2-methoxyethyl)-N⁶,2- dimethylpyrido[3,4-d]pyrimidine-4,6-diamine ¹H-NMR (500 MHz, DMSO-d6) δ [ppm]: 1.611 (4.34), 1.625 (4.39), 2.288 (11.61), 2.361 (0.41), 2.518 (1.48), 2.522 (1.10), 2.635 (0.42), 3.126 (11.46), 3.255 (16.00), 3.518 (1.53), 3.529 (3.58), 3.541 (1.81), 3.773 (0.55), 3.789 (0.62), 3.801 (1.10), 3.813 (0.49), 3.833 (0.56), 3.845 (1.11), 3.857 (0.56), 3.874 (0.55), 5.765 (0.68), 5.779 (1.06), 5.794 (0.68), 7.130 (0.84), 7.182 (2.92), 7.238 (1.77), 7.277 (0.73), 7.293 (1.59), 7.308 (0.89), 7.347 (0.77), 7.486 (0.55), 7.499 (0.95), 7.514 (0.49), 7.641 (0.52), 7.655 (0.97), 7.669 (0.49), 8.087 (0.55), 8.396 (1.12), 8.412 (1.10), 8.622 (3.55). |
| 155 | 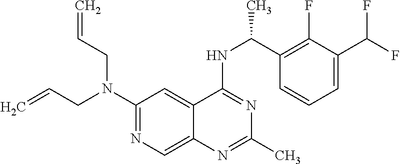 [SMILES: C=CCN(CC=C)c1cc2c(N[C@H](C)c3cccc(C(F)F)c3F)nc(C)nc2cn1] N-{(1R)-1-[3-(difluoromethyl)-2-fluorophenyl]ethyl}-2-methyl-N⁶,N⁶-di(prop-2-en-1- yl)pyrido[3,4-d]pyrimidine-4,6-diamine ¹H-NMR (400 MHz, DMSO-d6) δ [ppm]: 1.605 (5.42), 1.623 (5.43), 2.285 (16.00), 2.518 (1.27), 2.523 (0.79), 4.121 (0.77), 4.135 (0.80), 4.162 (1.82), 4.175 (1.80), 4.213 (1.75), 4.227 (1.82), 4.254 (0.77), 4.267 (0.76), 5.144 (2.28), 5.149 (2.59), 5.170 (4.42), 5.173 (4.54), 5.211 (2.75), 5.215 (2.46), 5.751 (0.85), 5.769 (1.31), 5.786 (0.86), 5.846 (0.65), 5.859 (1.45), 5.872 (1.13), 5.884 (1.54), 5.902 (1.42), 5.915 (0.98), 5.928 (1.21), 5.941 (0.52), 7.101 (1.21), 7.237 (2.67), 7.249 (3.50), 7.274 (0.94), 7.293 (2.03), 7.312 (1.17), 7.373 (1.09), 7.483 (0.74), 7.500 (1.23), 7.518 (0.62), 7.619 (0.67), 7.638 (1.20), 7.656 (0.62), 8.355 (1.37), 8.373 (1.32), 8.624 (4.61). |
| 156 | 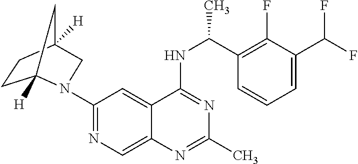 [SMILES: Cc1nc(N[C@H](C)c2cccc(C(F)F)c2F)c2cc(N3C[C@H]4CC[C@@H]3C4)ncc2n1] 6-[2-azabicyclo[2.2.1]heptan-2-yl]-N-{(1R)-1-[3-(difluoromethyl)-2-fluorophenyl]ethyl}-2- methylpyrido[3,4-d]pyrimidin-4-amine (mixture of stereoisomers) ¹H-NMR (400 MHz, DMSO-d6) δ [ppm]: 0.967 (0.73), 1.107 (5.81), 1.144 (0.58), 1.232 (0.45), 1.358 (0.59), 1.388 (1.20), 1.517 (1.34), 1.540 (1.71), 1.598 (5.75), 1.602 (5.87), 1.616 (6.06), 1.620 (5.72), 1.717 (3.42), 1.921 (0.43), 2.274 (15.01), 2.277 (16.00), 2.322 (0.98), 2.327 (1.37), 2.331 (1.00), 2.401 (0.66), 2.523 (5.92), 2.669 (3.13), 2.673 (3.03), 2.726 (0.49), 3.027 (0.98), 3.051 (1.74), 3.076 (1.05), 3.480 (1.05), 4.191 (0.47), 4.602 (2.68), 5.752 (0.94), 5.768 (1.41), 5.786 (0.92), 7.026 (2.80), 7.032 (2.68), 7.102 (1.77), 7.238 (3.62), 7.274 (0.93), 7.296 (2.09), 7.312 (1.21), 7.374 (1.57), 7.483 (1.05), 7.500 (1.80), 7.517 (0.94), 7.634 (0.99), 7.652 (1.80), 7.671 (0.96), 8.314 (1.38), 8.328 (1.36), 8.587 (6.31). |
| 157 | 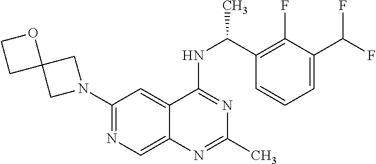 [SMILES: Cc1nc(N[C@H](C)c2cccc(C(F)F)c2F)c2cc(N3CC4(CCO4)C3)ncc2n1] N-{(1R)-1-[3-(difluoromethyl)-2-fluorophenyl]ethyl}-2-methyl-6-(1-oxa-6- azaspiro[3.3]heptan-6-yl)pyrido[3,4-d]pyrimidin-4-amine ¹H-NMR (400 MHz, DMSO-d6) δ [ppm]: 1.602 (5.43), 1.620 (5.49), 2.297 (16.00), 2.323 (0.56), 2.327 (0.60), 2.331 (0.46), 2.518 (3.78), 2.523 (2.61), 2.669 (0.50), 2.891 (1.54), 2.910 (3.32), 2.929 (1.63), 4.077 (1.50), 4.091 (1.61), 4.099 (1.94), 4.101 (1.94), 4.112 (1.88), 4.115 (1.86), 4.262 (1.83), 4.269 (1.90), 4.286 (1.49), 4.293 (1.51), 4.458 (1.78), 4.477 (3.62), 4.495 (1.74), 5.743 (0.86), 5.761 (1.31), 5.779 (0.86), 7.100 (1.22), 7.148 (3.78), 7.236 (2.53), 7.271 (0.92), 7.290 (2.04), 7.310 (1.20), 7.372 (1.09), 7.484 (0.74), 7.501 (1.25), 7.519 (0.64), 7.636 (0.67), 7.654 (1.23), 7.673 (0.63), 8.422 (1.40), 8.441 (1.36), 8.625 (4.57). |
| 158 | 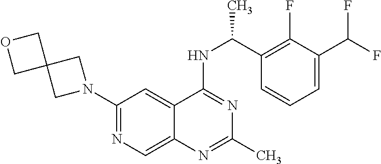 [SMILES: Cc1nc(N[C@H](C)c2cccc(C(F)F)c2F)c2cc(N3CC4(COC4)C3)ncc2n1] N-{(1R)-1-[3-(difluoromethyl)-2-fluorophenyl]ethyl}-2-methyl-6-(2-oxa-6- azaspiro[3.3]heptan-6-yl)pyrido[3,4-d]pyrimidin-4-amine ¹H-NMR (400 MHz, DMSO-d6) δ [ppm]: 1.604 (5.59), 1.622 (5.60), 2.302 (15.09), 2.518 (3.40), 2.522 (2.28), 4.167 (0.62), 4.190 (13.36), 4.213 (0.61), 4.765 (16.00), 5.749 (0.82), 5.767 (1.27), 5.785 (0.80), 7.098 (1.33), 7.142 (3.63), 7.235 (2.76), 7.276 (0.94), 7.295 (2.08), 7.314 (1.18), 7.370 (1.15), 7.487 (0.70), 7.504 (1.18), 7.521 (0.57), 7.634 (0.64), 7.652 (1.16), 7.671 (0.57), 8.624 (4.88), 8.626 (4.80). |
| @159 | 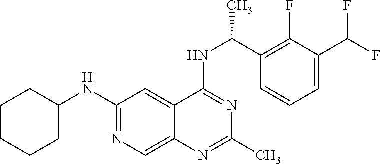 [SMILES: Cc1nc(N[C@H](C)c2cccc(C(F)F)c2F)c2cc(NC3CCCCC3)ncc2n1] N⁶-cyclohexyl-N⁴-{(1R)-1-[3-(difluoromethyl)-2-fluorophenyl]ethyl}-2-methylpyrido[3,4- d]pyrimidine-4,6-diamine ¹H-NMR (500 MHz, DMSO-d6) δ [ppm]: 1.181 (0.51), 1.205 (0.61), 1.229 (0.64), 1.241 (0.65), 1.267 (0.68), 1.287 (0.75), 1.307 (0.69), 1.331 (0.53), 1.338 (0.57), 1.368 (0.93), 1.387 (0.77), 1.595 (5.56), 1.609 (5.82), 1.636 (0.60), 1.749 (0.88), 1.929 (0.96), 1.948 (0.94), 2.265 (16.00), 2.514 (3.62), 2.518 (3.19), 2.522 (2.53), 3.614 (0.49), 3.623 (0.44), 3.632 (0.50), 5.729 (0.82), 5.743 (1.24), 5.757 (0.81), 6.265 (1.63), 6.282 (1.58), 6.988 (3.53), 7.124 (1.09), 7.233 (2.24), 7.268 (0.90), 7.284 (1.94), 7.299 (1.08), 7.342 (0.94), 7.480 (0.62), 7.492 (1.06), 7.507 (0.54), 7.645 (0.58), 7.661 (1.08), 7.675 (0.55), 8.088 (0.41), 8.246 (1.34), 8.260 (1.29), 8.529 (4.33). |
| 160 | 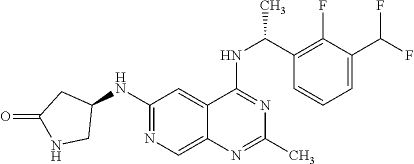 [SMILES: Cc1nc(N[C@H](C)c2cccc(C(F)F)c2F)c2cc(N[C@H]3CNC(=O)C3)ncc2n1] 4-{[4-({(1R)-1-[3-(difluoromethyl)-2-fluorophenyl]ethyl}amino)-2-methylpyrido[3,4- d]pyrimidin-6-yl]amino}pyrrolidin-2-one (mixture of stereoisomers) ¹H-NMR (400 MHz, DMSO-d6) δ [ppm]: 0.786 (0.47), 0.803 (0.71), 0.831 (1.18), 0.849 (1.59), 1.107 (1.71), 1.230 (5.41), 1.256 (1.76), 1.295 (0.76), 1.316 (0.47), 1.332 (0.76), 1.347 (2.18), 1.479 (0.47), 1.497 (0.53), 1.600 (6.53), 1.618 (6.59), 2.042 (0.59), 2.171 (0.76), 2.187 (0.82), 2.194 (0.88), 2.211 (1.59), 2.218 (0.76), 2.228 (1.47), 2.235 (1.06), 2.252 (1.06), 2.286 (16.00), 2.318 (0.82), 2.322 (1.59), 2.327 (1.94), 2.332 (1.47), 2.336 (0.76), 2.518 (8.00), 2.522 (4.88), 2.640 (0.82), 2.651 (0.88), 2.660 (1.59), 2.664 (1.65), 2.669 (2.35), 2.678 (0.88), 2.681 (1.00), 2.692 (0.88), 2.702 (0.82), 2.713 (0.82), 3.103 (0.65), 3.117 (0.71), 3.128 (1.29), 3.141 (1.35), 3.152 (0.76), 3.166 (0.71), 3.408 (0.47), 3.504 (0.76), 3.510 (0.76), 3.690 (0.71), 3.697 (0.71), 3.708 (0.88), 3.714 (1.29), 3.731 (0.71), 3.738 (0.65), 4.403 (0.59), 4.421 (0.94), 4.437 (0.94), 4.454 (0.53), 5.741 (0.88), 5.759 (1.41), 5.777 (0.94), 6.906 (1.41), 6.920 (1.35), 7.082 (3.94), 7.100 (1.71), 7.235 (3.47), 7.273 (1.12), 7.292 (2.29), 7.311 (1.35), 7.371 (1.47), 7.482 (1.06), 7.499 (1.71), 7.517 (0.88), 7.641 (0.94), 7.658 (1.65), 7.678 (0.94), 7.694 (1.94), 8.357 (1.35), 8.375 (1.35), 8.572 (6.06). |
| 161 | 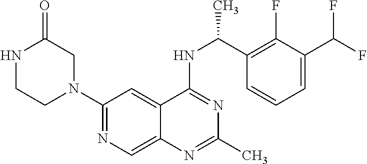 [SMILES: Cc1nc(N[C@H](C)c2cccc(C(F)F)c2F)c2cc(N3CCNC(=O)C3)ncc2n1] 4-[4-({(1R)-1-[3-(difluoromethyl)-2-fluorophenyl]ethyl}amino)-2-methylpyrido[3,4- d]pyrimidin-6-yl]piperazin-2-one LC-MS ( ): Rₜ = min; MS ( ): m/z = ¹H-NMR (400 MHz, DMSO-d6) δ [ppm]: 1.616 (2.21), 1.634 (2.19), 2.311 (6.74), 2.540 (16.00), 3.369 (0.51), 3.848 (0.67), 3.862 (0.88), 3.874 (0.60), 4.040 (1.54), 4.048 (1.54), 5.769 (0.52), 7.104 (0.50), 7.240 (1.07), 7.299 (0.84), 7.319 (0.48), 7.376 (0.44), 7.409 (1.35), 7.506 (0.49), 7.651 (0.48), 8.181 (0.67), 8.496 (0.56), 8.514 (0.54), 8.690 (2.08). |
| 162 | 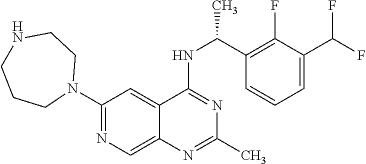 [SMILES: Cc1nc(N[C@H](C)c2cccc(C(F)F)c2F)c2cc(N3CCCNCC3)ncc2n1] 6-(1,4-diazepan-1-yl)-N-{(1R)-1-[3-(difluoromethyl)-2-fluorophenyl]ethyl}-2- methylpyrido[3,4-d]pyrimidin-4-amine LC-MS ( ): Rₜ = min; MS ( ): m/z = |
| 163 | 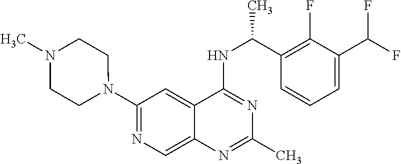 [SMILES: Cc1nc(N[C@H](C)c2cccc(C(F)F)c2F)c2cc(N3CCN(C)CC3)ncc2n1] N-{(1R)-1-[3-(difluoromethyl)-2-fluorophenyl]ethyl}-2-methyl-6-(4-methylpiperazin-1- yl)pyrido[3,4-d]pyrimidin-4-amine LC-MS ( ): Rₜ = min; MS ( ): m/z = ¹H-NMR (400 MHz, DMSO-d6) δ [ppm]: 1.605 (5.33), 1.623 (5.37), 2.253 (11.74), 2.303 (16.00), 2.323 (0.63), 2.327 (0.69), 2.331 (0.51), 2.465 (2.95), 2.478 (4.61), 2.518 (3.66), 2.523 (2.52), 2.665 (0.42), 2.669 (0.55), 3.545 (2.74), 3.557 (3.67), 3.569 (2.74), 5.751 (0.82), 5.769 (1.27), 5.787 (0.81), 7.103 (1.19), 7.239 (2.48), 7.277 (0.90), 7.296 (2.00), 7.315 (1.16), 7.374 (1.05), 7.440 (3.30), 7.487 (0.70), 7.505 (1.19), 7.522 (0.60), 7.633 (0.66), 7.652 (1.19), 7.669 (0.60), 8.430 (1.36), 8.448 (1.30), 8.658 (4.87). |
| 164 | 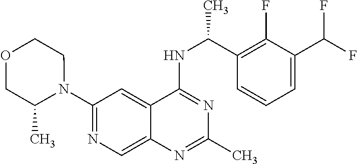 [SMILES: Cc1nc(N[C@H](C)c2cccc(C(F)F)c2F)c2cc(N3CCOC[C@H]3C)ncc2n1] N-{(1R)-1-[3-(difluoromethyl)-2-fluorophenyl]ethyl}-2-methyl-6-[(3R)-3- methylmorpholin-4-yl]pyrido[3,4-d]pyrimidin-4-amine LC-MS ( ): Rₜ = min; MS ( ): m/z = ¹H-NMR (400 MHz, DMSO-d6) δ [ppm]: 1.121 (6.30), 1.137 (6.40), 1.610 (5.69), 1.627 (5.70), 2.289 (0.61), 2.304 (16.00), 2.539 (3.43), 3.130 (0.47), 3.152 (0.83), 3.161 (0.85), 3.183 (0.56), 3.192 (0.48), 3.523 (0.46), 3.530 (0.62), 3.552 (0.88), 3.559 (0.95), 3.582 (0.53), 3.589 (0.46), 3.688 (0.65), 3.695 (0.71), 3.716 (1.26), 3.723 (1.19), 3.774 (1.97), 3.802 (1.86), 3.833 (0.82), 3.991 (0.82), 4.000 (0.88), 4.019 (0.77), 4.027 (0.71), 4.485 (0.72), 4.490 (0.70), 4.502 (0.72), 4.506 (0.70), 5.753 (0.86), 5.770 (1.33), 5.788 (0.85), 7.101 (1.28), 7.237 (2.68), 7.273 (0.98), 7.292 (2.12), 7.312 (1.24), 7.364 (3.53), 7.372 (1.45), 7.485 (0.74), 7.502 (1.26), 7.520 (0.62), 7.633 (0.68), 7.651 (1.23), 7.669 (0.61), 8.423 (1.45), 8.441 (1.40), 8.682 (5.43). |
| 165 | 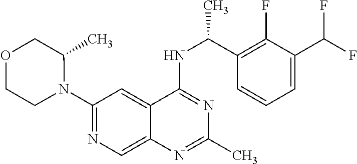 [SMILES: Cc1nc(N[C@H](C)c2cccc(C(F)F)c2F)c2cc(N3CCOC[C@@H]3C)ncc2n1] N-{(1R)-1-[3-(difluoromethyl)-2-fluorophenyl]ethyl}-2-methyl-6-[(3S)-3- methylmorpholin-4-yl]pyrido[3,4-d]pyrimidin-4-amine LC-MS ( ): Rₜ = min; MS ( ): m/z = |
| 166 | 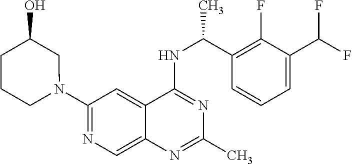 [SMILES: Cc1nc(N[C@H](C)c2cccc(C(F)F)c2F)c2cc(N3CCC[C@@H](O)C3)ncc2n1] (3R)-1-[4-({(1R)-1-[3-(difluoromethyl)-2-fluorophenyl]ethyl}amino)-2-methylpyrido[3,4- d]pyrimidin-6-yl]piperidin-3-ol LC-MS ( ): Rₜ = min; MS ( ): m/z = ¹H-NMR (400 MHz, DMSO-d6) δ [ppm]: 1.605 (1.80), 1.623 (1.82), 2.291 (4.92), 2.522 (0.88), 2.539 (16.00), 4.931 (0.78), 4.943 (0.78), 5.767 (0.44), 7.238 (0.82), 7.297 (0.68), 7.419 (1.14), 7.502 (0.43), 7.649 (0.42), 8.459 (0.46), 8.477 (0.45), 8.627 (1.71). |
| 167 | 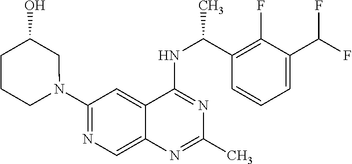 [SMILES: Cc1nc(N[C@H](C)c2cccc(C(F)F)c2F)c2cc(N3CCC[C@H](O)C3)ncc2n1] (3S)-1-[4-({(1R)-1-[3-(difluoromethyl)-2-fluorophenyl]ethyl}amino)-2-methylpyrido[3,4- d]pyrimidin-6-yl]piperidin-3-ol LC-MS ( ): Rₜ = min; MS ( ): m/z = ¹H-NMR (400 MHz, DMSO-d6) δ [ppm]: 1.103 (0.55), 1.107 (4.74), 1.232 (0.78), 1.375 (0.53), 1.384 (0.49), 1.399 (0.48), 1.408 (0.60), 1.503 (0.45), 1.532 (0.48), 1.605 (5.28), 1.623 (5.32), 1.773 (0.48), 1.782 (0.69), 1.791 (0.55), 1.805 (0.41), 1.815 (0.49), 1.901 (0.61), 1.931 (0.50), 1.940 (0.51), 1.960 (0.48), 2.290 (16.00), 2.332 (0.72), 2.518 (3.53), 2.522 (2.23), 2.539 (0.69), 2.673 (0.68), 2.729 (1.10), 2.751 (1.12), 2.759 (1.08), 2.782 (1.05), 2.883 (0.41), 2.888 (0.56), 2.916 (0.78), 2.942 (0.52), 2.949 (0.40), 3.565 (0.55), 4.111 (0.65), 4.143 (0.62), 4.275 (0.61), 4.285 (0.61), 4.305 (0.61), 4.315 (0.56), 4.941 (0.75), 4.951 (0.74), 5.747 (0.77), 5.765 (1.22), 5.782 (0.77), 7.102 (1.20), 7.238 (2.53), 7.276 (0.88), 7.295 (1.93), 7.314 (1.12), 7.374 (1.05), 7.424 (3.08), 7.483 (0.65), 7.501 (1.10), 7.518 (0.54), 7.629 (0.61), 7.647 (1.08), 7.665 (0.55), 8.380 (0.42), 8.464 (1.28), 8.483 (1.23), 8.628 (4.82). |
| 168 | 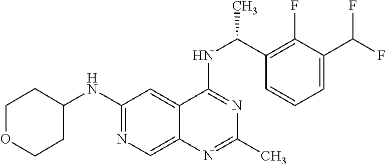 [SMILES: Cc1nc(N[C@H](C)c2cccc(C(F)F)c2F)c2cc(NC3CCOCC3)ncc2n1] N⁴-{(1R)-1-[3-(difluoromethyl)-2-fluorophenyl]ethyl}-2-methyl-N⁶-(oxan-4-yl)pyrido[3,4- d]pyrimidine-4,6-diamine LC-MS ( ): Rₜ = min; MS ( ): m/z = |
| 169 | 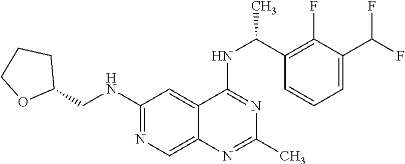 [SMILES: Cc1nc(N[C@H](C)c2cccc(C(F)F)c2F)c2cc(NC[C@H]3CCCO3)ncc2n1] N⁴-{(1R)-1-[3-(difluoromethyl)-2-fluorophenyl]ethyl}-2-methyl-N⁶-{[(2R)-oxolan-2- yl]methyl}pyrido[3,4-d]pyrimidine-4,6-diamine LC-MS ( ): Rₜ = min; MS ( ): m/z = ¹H-NMR (400 MHz, DMSO-d6) δ [ppm]: 1.231 (0.61), 1.591 (5.76), 1.609 (5.88), 1.619 (1.12), 1.641 (0.88), 1.649 (0.81), 1.653 (0.61), 1.665 (0.55), 1.670 (0.87), 1.688 (0.48), 1.804 (0.44), 1.824 (0.74), 1.840 (1.12), 1.857 (1.00), 1.865 (0.91), 1.87 (0.78), 1.882 (0.68), 1.886 (0.72), 1.903 (0.67), 1.943 (0.54), 1.960 (0.68), 1.973 (0.83), 1.981 (0.54), 1.989 (0.60), 1.993 (0.67), 2.004 (0.46), 2.276 (16.00), 2.522 (3.68), 2.673 (0.85), 3.275 (0.76), 3.292 (1.37), 3.308 (2.34), 3.358 (1.43), 3.376 (0.76), 3.629 (0.71), 3.648 (1.34), 3.665 (1.53), 3.683 (0.82), 3.779 (0.88), 3.796 (1.43), 3.812 (1.16), 3.832 (0.63), 4.060 (1.10), 4.076 (1.69), 4.092 (1.07), 5.734 (0.91), 5.751 (1.38), 5.769 (0.89), 6.365 (0.88), 6.380 (1.73), 6.395 (0.84), 7.084 (3.91), 7.099 (1.33), 7.235 (2.64), 7.270 (0.98), 7.289 (2.15), 7.308 (1.25), 7.371 (1.15), 7.479 (0.80), 7.496 (1.33), 7.514 (0.67), 7.642 (0.73), 7.660 (1.30), 7.677 (0.64), 8.088 (0.45), 8.330 (1.47), 8.349 (1.42), 8.536 (4.83). |
| 170 | 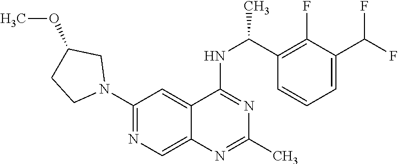 [SMILES: CO[C@H]1CCN(c2cc3c(N[C@H](C)c4cccc(C(F)F)c4F)nc(C)nc3cn2)C1] N-{(1R)-1-[3-(difluoromethyl)-2-fluorophenyl]ethyl}-6-[(3S)-3-methoxypyrrolidin-1-yl]-2- methylpyrido[3,4-d]pyrimidin-4-amine ¹H-NMR (400 MHz, DMSO-d6) δ [ppm]: 1.234 (0.21), 1.605 (1.03), 1.614 (1.16), 1.621 (1.19), 1.630 (1.04), 2.126 (0.66), 2.282 (2.29), 2.292 (2.23), 2.326 (0.24), 2.669 (0.23), 3.297 (3.04), 3.307 (2.79), 3.327 (16.00), 3.338 (14.25), 3.445 (0.26), 3.463 (0.30), 3.581 (1.20), 4.141 (0.45), 5.782 (0.29), 7.075 (0.69), 7.084 (0.70), 7.098 (0.22), 7.234 (0.39), 7.243 (0.38), 7.275 (0.20), 7.286 (0.39), 7.294 (0.39), 7.305 (0.25), 7.380(0.18), 7.498 (0.36), 7.656 (0.36), 8.083 (0.18), 8.094 (0.17), 8.361 (0.35), 8.372 (0.34), 8.625 (0.74), 8.634 (0.72). |
| 171 | 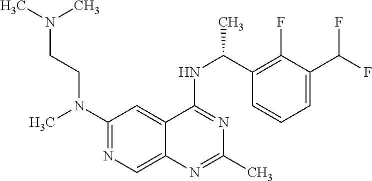 [SMILES: Cc1nc(N[C@H](C)c2cccc(C(F)F)c2F)c2cc(N(C)CCN(C)C)ncc2n1] N⁴-{(1R)-1-[3-(difluoromethyl)-2-fluorophenyl]ethyl}-N⁶-[2-(dimethylamino)ethyl]-N⁶,2- dimethylpyrido[3,4-d]pyrimidine-4,6-diamine ¹H-NMR (400 MHz, DMSO-d6) δ [ppm]: 0.904 (0.54), 1.229 (0.70), 1.606 (2.61), 1.624 (2.60), 2.179 (16.00), 2.287 (7.73), 2.301 (0.51), 2.404 (0.70), 2.422 (1.59), 2.439 (0.76), 2.518 (1.72), 2.523 (1.24), 3.096 (7.05), 3.746 (0.62), 3.762 (0.44), 3.778 (0.62), 5.778 (0.59), 7.102 (0.60), 7.148 (1.57), 7.237 (1.23), 7.272 (0.44), 7.291 (0.95), 7.310 (0.54), 7.373 (0.53), 7.499 (0.55), 7.656 (0.53), 8.088 (0.91), 8.383 (0.61), 8.401 (0.59), 8.616 (2.19). |
| 172 | 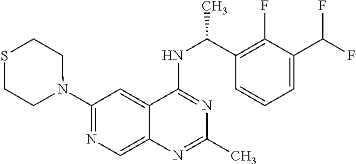 [SMILES: Cc1nc(N[C@H](C)c2cccc(C(F)F)c2F)c2cc(N3CCSCC3)ncc2n1] N-{(1R)-1-[3-(difluoromethyl)-2-fluorophenyl]ethyl}-2-methyl-6-(thiomorpholin-4- yl)pyrido[3,4-d]pyrimidin-4-amine ¹H-NMR (400 MHz, DMSO-d6) δ [ppm]: 1.611 (4.94), 1.629 (4.89), 2.299 (16.00), 2.322 (0.46), 2.327 (0.63), 2.332 (0.46), 2.518 (2.45), 2.523 (1.70), 2.665 (0.57), 2.669 (0.89), 2.679 (3.02), 2.685 (2.79), 2.691 (2.95), 2.697 (2.81), 2.704 (3.00), 3.970 (2.96), 3.976 (2.66), 3.983 (3.10), 3.988 (2.66), 3.995 (2.86), 5.749 (0.74), 5.767 (1.13), 5.785 (0.72), 7.104 (1.09), 7.240 (2.33), 7.277 (0.80), 7.297 (1.76), 7.316 (1.02), 7.376 (0.97), 7.436 (2.93), 7.487 (0.60), 7.504 (1.02), 7.522 (0.49), 7.630 (0.56), 7.649 (1.00), 7.667 (0.50), 8.409 (1.19), 8.427 (1.15), 8.663 (4.41). |
| 173 | 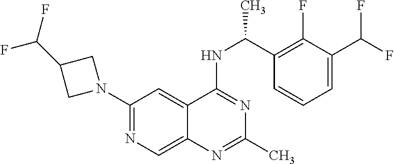 [SMILES: Cc1nc(N[C@H](C)c2cccc(C(F)F)c2F)c2cc(N3CC(C(F)F)C3)ncc2n1] 6-[3-(difluoromethyl)azetidin-1-yl]-N-{(1R)-1-[3-(difluoromethyl)-2-fluorophenyl]ethyl}- 2-methylpyrido[3,4-d]pyrimidin-4-amine ¹H-NMR (400 MHz, DMSO-d6) δ [ppm]: 1.232 (0.64), 1.646 (10.37), 1.663 (10.32), 2.415 (16.00), 2.518 (5.58), 2.523 (4.10), 2.679 (0.46), 2.995 (0.67), 3.353 (2.25), 3.973 (1.97), 3.986 (3.02), 3.995 (3.28), 4.006 (3.51), 4.019 (2.25), 4.157 (2.12), 4.167 (2.25), 4.178 (4.02), 4.188 (3.99), 4.199 (1.74), 4.209 (1.61), 5.824 (1.38), 5.842 (2.12), 5.860 (1.36), 6.280 (1.05), 6.291 (0.97), 6.422 (2.00), 6.432 (2.07), 6.562 (0.84), 6.573 (0.90), 7.103 (2.79), 7.240 (5.73), 7.269 (5.35), 7.315 (1.89), 7.335 (4.10), 7.354 (2.33), 7.375 (2.48), 7.528 (1.43), 7.546 (2.38), 7.563 (1.15), 7.684 (1.31), 7.703 (2.33), 7.721 (1.15), 8.701 (9.09). |
| 174 | 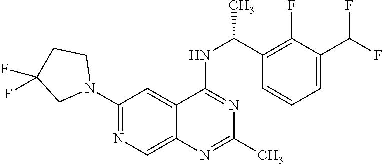 [SMILES: Cc1nc(N[C@H](C)c2cccc(C(F)F)c2F)c2cc(N3CCC(F)(F)C3)ncc2n1] N-{(1R)-1-[3-(difluoromethyl)-2-fluorophenyl]ethyl}-6-(3,3-difluoropyrrolidin-1-yl)-2- methylpyrido[3,4-d]pyrimidin-4-amine ¹H-NMR (400 MHz, DMSO-d6) δ [ppm]: 1.231 (0.82), 1.614 (6.03), 1.632 (6.00), 1.921 (2.88), 2.293 (0.42), 2.309 (16.00), 2.323 (0.91), 2.327 (1.06), 2.332 (0.76), 2.518 (3.21), 2.523 (2.06), 2.582 (0.64), 2.599 (0.97), 2.618 (1.24), 2.635 (1.00), 2.654 (0.61), 2.660 (0.52), 2.665 (0.82), 2.669 (1.21), 2.673 (0.91), 3.699 (1.00), 3.705 (0.97), 3.718 (1.79), 3.722 (1.70), 3.735 (0.91), 3.741 (0.94), 3.887 (1.00), 3.920 (1.88), 3.952 (0.97), 5.760 (0.82), 5.779 (1.24), 5.796 (0.79), 7.102 (1.21), 7.209 (3.42), 7.220 (0.39), 7.238 (2.55), 7.275 (1.12), 7.294 (2.12), 7.314 (1.15), 7.374 (1.09), 7.489 (0.73), 7.506 (1.24), 7.524 (0.61), 7.638 (0.67), 7.656 (1.21), 7.674 (0.61), 8.400 (1.30), 8.418 (1.27), 8.681 (4.64), 8.768 (0.48). |
| 175 | 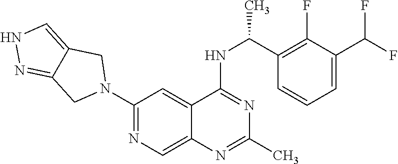 [SMILES: Cc1nc(N[C@H](C)c2cccc(C(F)F)c2F)c2cc(N3Cc4c[nH]nc4C3)ncc2n1] N-{(1R)-1-[3-(difluoromethyl)-2-fluorophenyl]ethyl}-6-(2,6-dihydropyrrolo[3,4-c]pyrazol- 5(4H)-yl)-2-methylpyrido[3,4-d]pyrimidin-4-amine ¹H-NMR (500 MHz, DMSO-d6) δ [ppm]: 1.228 (0.42), 1.633 (5.79), 1.647 (5.65), 1.752 (0.46), 2.074 (7.30), 2.312 (16.00), 2.323 (0.52), 2.514 (2.30), 2.518 (2.18), 2.522 (1.80), 2.539 (3.39), 4.589 (4.23), 5.783 (0.91), 5.797 (1.35), 5.812 (0.89), 7.136 (1.18), 7.212 (3.30), 7.245 (2.55), 7.289 (1.06), 7.305 (2.15), 7.320 (1.23), 7.354 (1.04), 7.496 (0.80), 7.509 (1.25), 7.523 (0.63), 7.648 (1.57), 7.667 (0.73), 7.682 (1.23), 7.697 (0.61), 8.401 (1.24), 8.416 (1.20), 8.710 (4.69), 12.725 (1.15). |
| 176 | 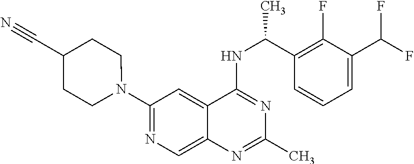 [SMILES: Cc1nc(N[C@H](C)c2cccc(C(F)F)c2F)c2cc(N3CCC(C#N)CC3)ncc2n1] 1-[4-({(1R)-1-[3-(difluoromethyl)-2-fluorophenyl]ethyl}amino)-2-methylpyrido[3,4- d]pyrimidin-6-yl]piperidine-4-carbonitrile ¹H-NMR (400 MHz, DMSO-d6) δ [ppm]: 1.107 (5.37), 1.610 (5.07), 1.627 (5.05), 1.794 (0.44), 1.803 (0.84), 1.814 (0.82), 1.826 (0.86), 1.836 (1.01), 1.845 (0.60), 1.858 (0.50), 2.007 (0.91), 2.038 (0.68), 2.304 (16.00), 2.318 (0.53), 2.323 (0.85), 2.327 (1.11), 2.332 (0.78), 2.518 (3.86), 2.523 (2.58), 2.665 (0.74), 2.669 (1.05), 2.673 (0.72), 3.128 (0.47), 3.138 (0.65), 3.149 (0.88), 3.160 (0.65), 3.171 (0.43), 3.395 (0.72), 3.401 (0.60), 3.422 (1.02), 3.448 (0.77), 3.870 (0.71), 3.879 (0.94), 3.886 (0.92), 3.894 (0.83), 3.903 (0.76), 3.912 (0.79), 3.919 (0.85), 3.927 (0.61), 4.190 (0.48), 5.751 (0.77), 5.768 (1.19), 5.786 (0.75), 7.103 (1.17), 7.239 (2.44), 7.278 (0.85), 7.297 (1.87), 7.316 (1.09), 7.374 (1.02), 7.475 (3.09), 7.488 (0.70), 7.506 (1.08), 7.524 (0.52), 7.633 (0.58), 7.651 (1.05), 7.670 (0.53), 8.429 (1.25), 8.447 (1.20), 8.667 (4.60). |
| 177 | 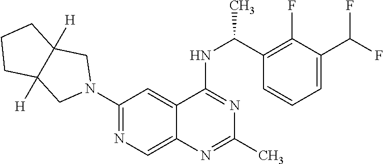 [SMILES: Cc1nc(N[C@H](C)c2cccc(C(F)F)c2F)c2cc(N3CC4CCCC4C3)ncc2n1] N-{(1R)-1-[3-(difluoromethyl)-2-fluorophenyl]ethyl}-6-[hexahydrocyclopenta[c]pyrrol- 2(1H)-yl]-2-methylpyrido[3,4-d]pyrimidin-4-amine (mixture of stereoisomers) ¹H-NMR (400 MHz, DMSO-d6) δ [ppm]: 1.504 (0.96), 1.519 (1.04), 1.534 (0.90), 1.545 (0.91), 1.562 (0.57), 1.576 (0.72), 1.602 (5.69), 1.619 (5.76), 1.696 (0.59), 1.714 (0.94), 1.731 (0.73), 1.744 (0.63), 1.762 (0.46), 1.809 (0.48), 1.827 (1.04), 1.845 (1.12), 1.857 (1.12), 1.875 (0.80), 2.290 (16.00), 2.326 (0.47), 2.518 (3.11), 2.522 (2.16), 2.669 (0.41), 2.808 (1.37), 2.817 (1.40), 3.240 (0.88), 3.249 (1.59), 3.259 (1.02), 3.266 (1.15), 3.276 (1.81), 3.285 (0.98), 3.621 (0.70), 3.640 (1.44), 3.646 (1.13), 3.661 (1.13), 3.667 (1.32), 3.687 (0.61), 5.759 (0.82), 5.777 (1.28), 5.795 (0.82), 7.102 (4.38), 7.237 (2.49), 7.272 (0.90), 7.292 (2.00), 7.311 (1.17), 7.373 (1.07), 7.484 (0.70), 7.500 (1.20), 7.517 (0.61), 7.636 (0.65), 7.655 (1.18), 7.672 (0.60), 8.356 (1.36), 8.375 (1.32), 8.622 (4.68). |
| 178 | 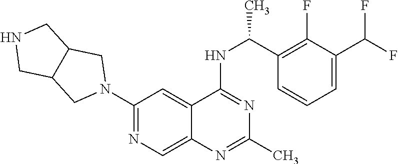 [SMILES: Cc1nc(N[C@H](C)c2cccc(C(F)F)c2F)c2cc(N3CC4CNCC4C3)ncc2n1] N-{(1R)-1-[3-(difluoromethyl)-2-fluorophenyl]ethyl]-6-[hexahydropyrrolo[3,4-c]pyrrol- 2(1H)-yl]-2-methylpyrido[3,4-d]pyrimidin-4-amine (mixture of stereoisomers) ¹H-NMR (400 MHz, DMSO-d6) δ [ppm]: 1.603 (5.39), 1.621 (5.43), 2.292 (16.00), 2.323 (0.66), 2.327 (0.78), 2.332 (0.61), 2.518 (4.63), 2.523 (3.41), 2.540 (2.81), 2.669 (1.43), 2.674 (1.56), 2.703 (1.43), 2.895 (1.13), 2.962 (1.25), 2.979 (0.98), 2.988 (1.20), 3.006 (0.80), 3.321 (1.91), 3.629 (0.59), 3.650 (1.03), 3.676 (1.06), 3.696 (0.66), 5.761 (0.81), 5.779 (1.25), 5.797 (0.80), 7.101 (1.39), 7.131 (2.44), 7.238 (2.69), 7.274 (0.92), 7.293 (1.96), 7.312 (1.14), 7.373 (1.12), 7.484 (0.74), 7.502 (1.22), 7.518 (0.61), 7.636 (0.66), 7.654 (1.18), 7.672 (0.61), 8.385 (1.13), 8.404 (1.04), 8.630 (4.24). |
| 179 | 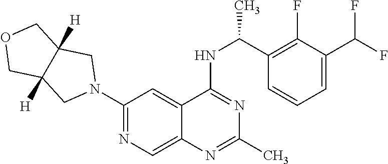 [SMILES: Cc1nc(N[C@H](C)c2cccc(C(F)F)c2F)c2cc(N3C[C@H]4COC[C@H]4C3)ncc2n1] N-{(1R)-1-[3-(difluoromethyl)-2-fluorophenyl]ethyl}-2-methyl-6-[(3aR,6aS)-tetrahydro- 1H-furo[3,4-c]pyrrol-5(3H)-yl]pyrido[3,4-d]pyrimidin-4-amine ¹H-NMR (400 MHz, DMSO-d6) δ [ppm]: 1.605 (5.61), 1.623 (5.58), 2.296 (16.00), 2.518 (2.47), 2.523 (1.53), 3.079 (1.60), 3.424 (1.78), 3.451 (2.23), 3.580 (1.11), 3.590 (1.96), 3.600 (2.49), 3.612 (2.91), 3.633 (1.23), 3.642 (1.26), 3.661 (0.62), 3.870 (1.75), 3.886 (2.04), 3.891 (1.89), 3.908 (1.44), 5.760 (0.88), 5.777 (1.34), 5.795 (0.85), 7.102 (1.28), 7.153 (3.68), 7.238 (2.59), 7.273 (0.97), 7.292 (2.12), 7.312 (1.22), 7.374 (1.12), 7.485 (0.76), 7.502 (1.29), 7.519 (0.63), 7.638 (0.70), 7.655 (1.26), 7.674 (0.63), 8.383 (1.47), 8.401 (1.40), 8.639 (5.03). |
| 180 | 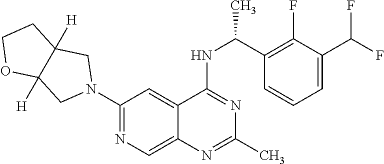 [SMILES: Cc1nc(N[C@H](C)c2cccc(C(F)F)c2F)c2cc(N3CC4CCOC4C3)ncc2n1] N-{(1R)-1-[3-(difluoromethyl)-2-fluorophenyl]ethyl]-6-[(3aRS,6aRS)-hexahydro-5H- furo[2,3-c]pyrrol-5-yl]-2-methylpyrido[3,4-d]pyrimidin-4-amine (mixture of stereisomers) ¹H-NMR (400 MHz, DMSO-d6) δ [ppm]: 1.230 (0.44), 1.606 (7.20), 1.624 (7.13), 1.838 (0.63), 1.846 (0.72), 1.853 (0.68), 2.111 (0.96), 2.130 (1.08), 2.142 (0.88), 2.149 (0.50), 2.161 (0.85), 2.295 (16.00), 2.323 (0.58), 2.327 (0.56), 2.331 (0.44), 2.394 (1.10), 2.464 (0.62), 2.469 (0.62), 2.473 (0.73), 2.478 (1.08), 2.518 (2.01), 2.523 (1.36), 2.529 (0.51), 2.665 (0.40), 2.669 (0.54), 3.058 (0.60), 3.072 (0.78), 3.093 (0.64), 3.314 (0.42), 3.356 (0.91), 3.360 (1.02), 3.369 (0.81), 3.383 (0.94), 3.387 (0.94), 3.396 (0.85), 3.478 (0.59), 3.490 (0.69), 3.499 (0.64), 3.507 (0.99), 3.519 (0.80), 3.528 (0.88), 3.541 (0.77), 3.608 (0.72), 3.623 (0.81), 3.629 (0.92), 3.634 (0.84), 3.644 (0.88), 3.649 (0.87), 3.656 (0.63), 3.671 (0.59), 3.686 (1.20), 3.709 (1.28), 3.716 (1.03), 3.731 (0.79), 3.738 (1.09), 3.751 (1.45), 3.763 (1.44), 3.771 (0.96), 3.783 (0.80), 3.849 (0.44), 3.857 (0.50), 3.867 (0.96), 3.875 (1.00), 3.887 (0.83), 3.893 (0.78), 4.608 (0.91), 4.622 (1.54), 4.635 (0.85), 5.757 (1.11), 5.775 (1.68), 5.793 (1.05), 7.102 (1.59), 7.141 (3.32), 7.238 (3.33), 7.274 (1.14), 7.293 (2.48), 7.312 (1.43), 7.374 (1.41), 7.485 (0.91), 7.502 (1.58), 7.519 (0.83), 7.636 (0.84), 7.655 (1.54), 7.672 (0.78), 8.361 (1.78), 8.379 (1.71), 8.636 (6.07). |
| 181 | 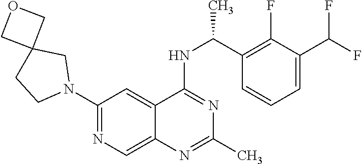 [SMILES: Cc1nc(N[C@H](C)c2cccc(C(F)F)c2F)c2cc(N3CCC4(COC4)C3)ncc2n1] N-{(1R)-1-[3-(difluoromethyl)-2-fluorophenyl]ethyl}-2-methyl-6-(2-oxa-6- azaspiro[3.4]octan-6-yl)pyrido[3,4-d]pyrimidin-4-amine LC-MS ( ): Rₜ = min; MS ( ): m/z = ¹H-NMR (400 MHz, DMSO-d6) δ [ppm]: 1.107 (0.66), 1.611 (4.86), 1.629 (4.85), 2.283 (2.65), 2.290 (16.00), 2.300 (3.25), 2.318 (1.71), 2.518 (0.77), 2.523 (0.50), 3.484 (0.86), 3.490 (0.88), 3.501 (1.48), 3.508 (1.55), 3.518 (0.85), 3.525 (0.81), 3.742 (4.49), 4.567 (3.44), 4.582 (5.36), 4.615 (2.88), 4.623 (2.87), 4.630 (1.85), 4.637 (1.78), 5.765 (0.72), 5.783 (1.13), 5.801 (0.71), 7.098 (3.28), 7.237 (2.43), 7.275 (0.83), 7.293 (1.84), 7.312 (1.06), 7.373 (1.02), 7.485 (0.61), 7.502 (1.06), 7.519 (0.50), 7.639 (0.55), 7.657 (1.02), 7.675 (0.50), 8.382 (1.23), 8.400 (1.18), 8.628 (4.34). |
| 182 | 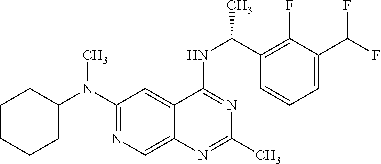 [SMILES: Cc1nc(N[C@H](C)c2cccc(C(F)F)c2F)c2cc(N(C)C3CCCCC3)ncc2n1] N⁶-cyclohexyl-N⁴-{(1R)-1-[3-(difluoromethyl)-2-fluorophenyl]ethyl}-N⁶,2- dimethylpyrido[3,4-d]pyrimidine-4,6-diamine ¹H-NMR (500 MHz, DMSO-d6) δ [ppm]: 0.905 (0.44), 1.131 (0.60), 1.157 (0.66), 1.372 (0.93), 1.397 (0.99), 1.423 (0.44), 1.519 (0.69), 1.526 (0.83), 1.543 (0.83), 1.550 (0.91), 1.561 (0.77), 1.609 (6.71), 1.623 (6.45), 1.661 (0.64), 1.789 (1.19), 1.815 (1.03), 2.284 (16.00), 2.358 (0.87), 2.361 (1.17), 2.365 (0.83), 2.460 (0.64), 2.518 (3.16), 2.522 (2.36), 2.631 (0.83), 2.635 (1.15), 2.639 (0.83), 2.922 (14.67), 4.663 (0.44), 4.678 (0.46), 4.686 (0.85), 4.693 (0.54), 4.709 (0.44), 5.766 (0.87), 5.781 (1.31), 5.795 (0.85), 7.114 (3.69), 7.128 (1.15), 7.237 (2.34), 7.274 (0.97), 7.290 (2.04), 7.305 (1.21), 7.346 (0.99), 7.484 (0.69), 7.497 (1.21), 7.511 (0.64), 7.643 (0.66), 7.657 (1.21), 7.671 (0.62), 8.088 (0.71), 8.379 (1.39), 8.394 (1.33), 8.635 (4.68). |
| 183 | 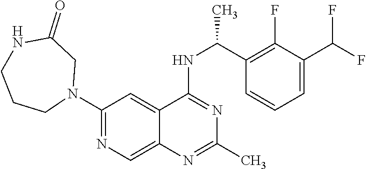 [SMILES: Cc1nc(N[C@H](C)c2cccc(C(F)F)c2F)c2cc(N3CCCNC(=O)C3)ncc2n1] 4-[4-({(1R)-1-[3-(difluoromethyl)-2-fluorophenyl]ethyl}amino)-2-methylpyrido[3,4- d]pyrimidin-6-yl]-1,4-diazepan-2-one ¹H-NMR (400 MHz, DMSO-d6) δ [ppm]: 0.859 (0.71), 0.967 (1.78), 1.107 (3.13), 1.144 (1.01), 1.231 (0.65), 1.348 (0.42), 1.472 (0.40), 1.614 (5.18), 1.631 (5.23), 1.832 (1.42), 2.097 (1.26), 2.292 (16.00), 2.331 (1.40), 2.518 (12.32), 2.523 (8.39), 3.181 (1.49), 3.202 (1.43), 3.845 (0.47), 3.865 (0.51), 3.880 (0.87), 3.936 (0.83), 3.952 (0.56), 3.971 (0.49), 4.375 (4.55), 5.756 (0.79), 5.774 (1.25), 5.793 (0.80), 7.104 (1.22), 7.240 (2.74), 7.269 (0.95), 7.289 (1.96), 7.308 (1.18), 7.376 (1.19), 7.403 (3.20), 7.466 (0.80), 7.479 (1.93), 7.500 (1.39), 7.518 (0.67), 7.645 (0.66), 7.662 (1.19), 7.682 (0.64), 8.422 (1.45), 8.441 (1.43), 8.636 (4.67). |
| 184 | 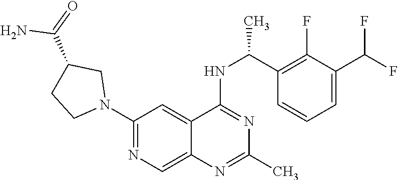 [SMILES: Cc1nc(N[C@H](C)c2cccc(C(F)F)c2F)c2cc(N3CC[C@H](C(N)=O)C3)ncc2n1] (3S)-1-[4-({(1R)-1-[3-(difluoromethyl)-2-fluorophenyl]ethyl}amino)-2-methylpyrido[3,4- d]pyrimidin-6-yl]pyrrolidine-3-carboxamide ¹H-NMR (400 MHz, DMSO-d6) δ [ppm]: 1.107 (0.61), 1.232 (0.69), 1.504 (0.57), 1.520 (0.61), 1.603 (4.99), 1.621 (5.04), 2.083 (0.41), 2.104 (0.49), 2.114 (0.73), 2.123 (0.41), 2.135 (0.61), 2.193 (0.77), 2.205 (0.65), 2.216 (0.97), 2.234 (0.61), 2.245 (0.45), 2.261 (1.02), 2.287 (12.10), 2.337 (0.49), 2.518 (4.91), 2.523 (3.17), 2.540 (16.00), 2.679 (0.45), 3.097 (0.57), 3.116 (0.81), 3.136 (0.57), 3.433 (0.45), 3.452 (0.69), 3.459 (0.73), 3.478 (0.73), 3.533 (0.61), 3.552 (0.65), 3.559 (0.97), 3.577 (0.81), 3.616 (0.41), 3.627 (1.14), 3.636 (0.73), 3.648 (1.46), 3.672 (0.97), 5.753 (0.61), 5.758 (0.57), 5.771 (0.97), 5.789 (0.65), 7.007 (1.22), 7.081 (3.29), 7.102 (1.22), 7.238 (2.48), 7.273 (1.06), 7.291 (2.03), 7.310 (1.18), 7.374 (1.06), 7.481 (0.73), 7.497 (1.22), 7.516 (0.81), 7.531 (1.22), 7.630 (0.69), 7.649 (1.14), 7.668 (0.57), 8.388 (1.14), 8.406 (1.10), 8.624 (4.67). |
| 185 | 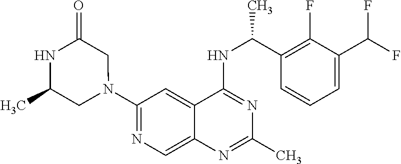 [SMILES: Cc1nc(N[C@H](C)c2cccc(C(F)F)c2F)c2cc(N3CC(=O)N[C@H](C)C3)ncc2n1] (6R)-4-[4-({(1R)-1-[3-(difluoromethyl)-2-fluorophenyl]ethyl}amino)-2-methylpyrido[3,4- d]pyrimidin-6-yl]-6-methylpiperazin-2-one ¹H-NMR (400 MHz, DMSO-d6) δ [ppm]: 1.170 (0.78), 1.186 (0.78), 1.617 (0.68), 1.635 (0.67), 2.309 (2.13), 2.518 (0.49), 2.539 (16.00), 7.395 (0.40), 8.688 (0.65). |
| 186 | 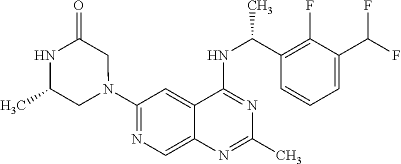 [SMILES: Cc1nc(N[C@H](C)c2cccc(C(F)F)c2F)c2cc(N3CC(=O)N[C@@H](C)C3)ncc2n1] (6S)-4-[4-({(1R)-1-[3-(difluoromethyl)-2-fluorophenyl]ethyl}amino)-2-methylpyrido[3,4- d]pyrimidin-6-yl]-6-methylpiperazin-2-one ¹H-NMR (400 MHz, DMSO-d6) δ [ppm]: 1.172 (5.86), 1.188 (5.88), 1.618 (5.21), 1.636 (5.18), 2.309 (16.00), 2.323 (1.15), 2.327 (1.40), 2.332 (0.99), 2.336 (0.44), 2.518 (4.91), 2.523 (3.20), 2.540 (0.50), 2.660 (0.41), 2.665 (0.94), 2.669 (1.31), 2.673 (0.93), 2.679 (0.42), 2.914 (0.71), 3.208 (0.88), 3.228 (1.01), 3.240 (0.96), 3.260 (1.05), 3.630 (0.58), 3.838 (1.77), 3.881 (2.24), 4.147 (1.82), 4.192 (1.44), 4.231 (0.81), 4.240 (0.88), 4.263 (0.82), 4.272 (0.74), 5.753 (0.79), 5.771 (1.24), 5.789 (0.78), 7.105 (1.18), 7.241 (2.55), 7.280 (0.94), 7.300 (1.99), 7.319 (1.16), 7.377 (1.10), 7.397 (3.08), 7.489 (0.67), 7.506 (1.15), 7.523 (0.59), 7.634 (0.61), 7.651 (1.11), 7.670 (0.57), 8.222 (1.97), 8.487 (1.27), 8.506 (1.23), 8.687 (4.78). |
| 187 | 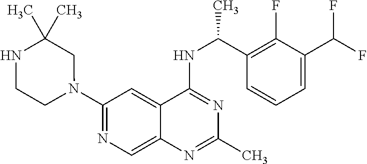 [SMILES: Cc1nc(N[C@H](C)c2cccc(C(F)F)c2F)c2cc(N3CCNC(C)(C)C3)ncc2n1] N-{(1R)-1-[3-(difluoromethyl)-2-fluorophenyl]ethyl}-6-(3,3-dimethylpiperazin-1-yl)-2- methylpyrido[3,4-d]pyrimidin-4-amine ¹H-NMR (400 MHz, DMSO-d6) δ [ppm]: 1.108 (13.66), 1.374 (0.48), 1.611 (3.37), 1.629 (3.36), 2.292 (9.71), 2.523 (0.66), 2.888 (0.85), 2.900 (1.68), 2.913 (0.94), 3.283 (0.51), 3.313 (1.78), 3.333 (16.00), 3.362 (0.54), 3.411 (0.44), 3.425 (0.69), 3.464 (0.66), 3.478 (0.41), 5.754 (0.51), 5.772 (0.80), 5.790 (0.51), 7.103 (0.74), 7.240 (1.54), 7.277 (0.58), 7.296 (1.26), 7.315 (0.73), 7.355 (2.10), 7.375 (0.70), 7.485 (0.44), 7.503 (0.76), 7.635 (0.41), 7.654 (0.75), 8.379 (0.87), 8.398 (0.84), 8.632 (3.16). |
| 188 | 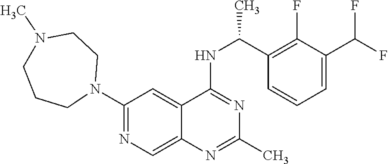 [SMILES: Cc1nc(N[C@H](C)c2cccc(C(F)F)c2F)c2cc(N3CCCN(C)CC3)ncc2n1] N-{(1R)-1-[3-(difluoromethyl)-2-fluorophenyl]ethyl}-2-methyl-6-(4-methyl-1,4-diazepan- 1-yl)pyrido[3,4-d]pyrimidin-4-amine ¹H-NMR (400 MHz, DMSO-d6) δ [ppm]: 1.052 (0.71), 1.608 (4.41), 1.625 (4.41), 1.974 (1.11), 2.283 (16.00), 2.286 (11.64), 2.300 (0.73), 2.318 (0.47), 2.323 (0.98), 2.327 (1.40), 2.332 (1.02), 2.336 (0.47), 2.518 (6.25), 2.523 (4.76), 2.539 (2.63), 2.653 (1.05), 2.664 (2.05), 2.669 (2.38), 2.673 (1.76), 3.429 (0.49), 3.656 (1.31), 3.672 (1.94), 3.687 (1.18), 3.845 (1.16), 3.857 (1.47), 3.868 (1.13), 5.760 (0.67), 5.778 (1.02), 5.796 (0.65), 7.102 (1.07), 7.155 (2.51), 7.238 (2.25), 7.275 (0.73), 7.293 (1.60), 7.312 (0.93), 7.374 (0.91), 7.483 (0.58), 7.500 (0.93), 7.518 (0.47), 7.632 (0.53), 7.649 (0.93), 7.670 (0.47), 8.188 (0.91), 8.375 (1.02), 8.393 (1.02), 8.616 (3.72). |
| 189 | 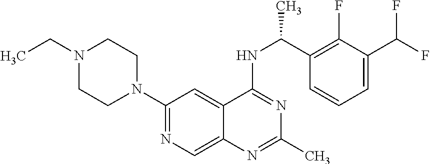 [SMILES: CCN1CCN(c2cc3c(N[C@H](C)c4cccc(C(F)F)c4F)nc(C)nc3cn2)CC1] N-{(1R)-1-[3-(difluoromethyl)-2-fluorophenyl]ethyl}-6-(4-ethylpiperazin-1-yl)-2- methylpyrido[3,4-d]pyrimidin-4-amine ¹H-NMR (400 MHz, DMSO-d6) δ [ppm]: 1.029 (4.02), 1.047 (9.02), 1.065 (5.10), 1.603 (6.88), 1.620 (7.67), 2.299 (16.00), 2.356 (1.49), 2.374 (4.07), 2.392 (4.35), 2.409 (1.75), 3.552 (7.19), 5.749 (1.19), 5.767 (1.88), 5.785 (1.37), 7.097 (1.39), 7.233 (2.83), 7.268 (1.27), 7.287 (2.73), 7.306 (1.77), 7.369 (1.30), 7.430 (4.45), 7.482 (1.21), 7.498 (2.09), 7.514 (1.27), 7.632 (1.09), 7.649 (2.03), 7.667 (1.23), 8.428 (2.02), 8.446 (2.16), 8.654 (5.78). |
| 190 | 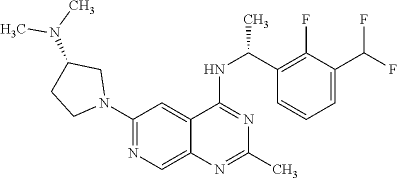 [SMILES: Cc1nc(N[C@H](C)c2cccc(C(F)F)c2F)c2cc(N3CC[C@H](N(C)C)C3)ncc2n1] N-{(1R)-1-[3-(difluoromethyl)-2-fluorophenyl]ethyl}-6-[(3S)-3-(dimethylamino)pyrrolidin- 1-yl]-2-methylpyrido[3,4-d]pyrimidin-4-amine ¹H-NMR (400 MHz, DMSO-d6) δ [ppm]: 1.609 (3.63), 1.626 (3.62), 1.862 (0.41), 1.868 (0.44), 2.198 (0.40), 2.214 (0.45), 2.238 (16.00), 2.285 (11.31), 2.518 (1.25), 2.522 (0.80), 2.841 (0.42), 3.188 (0.59), 3.209 (0.67), 3.213 (0.76), 3.234 (0.58), 3.419 (0.55), 3.436 (0.53), 3.644 (0.63), 3.725 (0.50), 3.743 (0.58), 3.750 (0.56), 3.768 (0.45), 5.758 (0.70), 5.776 (0.84), 5.794 (0.54), 7.049 (2.28), 7.101 (0.84), 7.237 (1.72), 7.271 (0.61), 7.290 (1.34), 7.309 (0.77), 7.373 (0.72), 7.482 (0.45), 7.498 (0.77), 7.634 (0.41), 7.652 (0.76), 8.340 (0.91), 8.358 (0.88), 8.627 (3.31). |
| 191 | 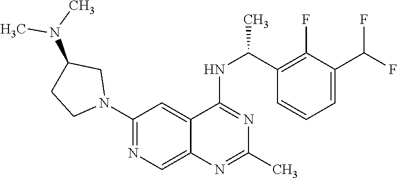 [SMILES: Cc1nc(N[C@H](C)c2cccc(C(F)F)c2F)c2cc(N3CC[C@@H](N(C)C)C3)ncc2n1] N-{(1R)-1-[3-(difluoromethyl)-2-fluorophenyl]ethyl}-6-[(3R)-3- (dimethylamino)pyrrolidin-1-yl]-2-methylpyrido[3,4-d]pyrimidin-4-amine ¹H-NMR (400 MHz, DMSO-d6) δ [ppm]: 1.610 (3.70), 1.627 (3.72), 1.867 (0.43), 1.872 (0.45), 2.198 (0.42), 2.212 (0.48), 2.238 (16.00), 2.285 (10.76), 2.518 (0.94), 2.522 (0.61), 2.844 (0.45), 3.175 (0.61), 3.196 (0.70), 3.200 (0.76), 3.221 (0.59), 3.401 (0.58), 3.418 (0.56), 3.667 (0.64), 3.741 (0.52), 3.758 (0.60), 3.766 (0.58), 3.784 (0.45), 5.765 (0.56), 5.783 (0.87), 5.801 (0.55), 7.050 (2.36), 7.100 (0.84), 7.237 (1.73), 7.270 (0.63), 7.289 (1.37), 7.308 (0.79), 7.372 (0.74), 7.480 (0.47), 7.497 (0.81), 7.634 (0.44), 7.653 (0.80), 8.345 (0.92), 8.363 (0.89), 8.626 (3.39). |
| 192 | 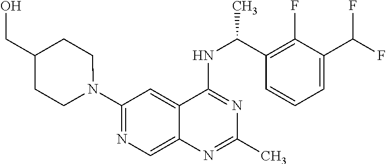 [SMILES: Cc1nc(N[C@H](C)c2cccc(C(F)F)c2F)c2cc(N3CCC(CO)CC3)ncc2n1] {1-[4-({(1R)-1-[3-(difluoromethyl)-2-fluorophenyl]ethyl}amino)-2-methylpyrido[3,4- d]pyrimidin-6-yl]piperidin-4-yl}methanol ¹H-NMR (400 MHz, DMSO-d6) δ [ppm]: 1.107 (1.84), 1.181 (0.87), 1.211 (0.96), 1.234 (0.44), 1.606 (5.39), 1.624 (5.55), 1.644 (0.60), 1.654 (0.48), 1.786 (1.23), 1.813 (1.13), 2.293 (16.00), 2.327 (0.47), 2.518 (2.08), 2.522 (1.30), 2.669 (0.44), 2.796 (0.85), 2.828 (1.60), 2.859 (0.84), 3.292 (1.87), 3.307 (2.96), 3.321 (2.54), 4.392 (1.16), 4.421 (1.09), 4.502 (1.27), 4.515 (3.05), 4.528 (1.22), 5.751 (0.80), 5.770 (1.24), 5.787 (0.80), 7.102 (1.20), 7.238 (2.51), 7.276 (0.90), 7.295 (1.95), 7.315 (1.14), 7.374 (1.07), 7.414 (3.17), 7.485 (0.69), 7.502 (1.16), 7.520 (0.57), 7.633 (0.63), 7.651 (1.14), 7.670 (0.57), 8.413 (1.34), 8.430 (1.28), 8.638 (5.01). |
| 193 | 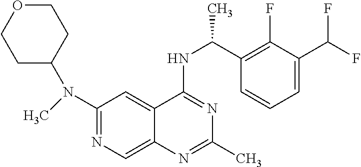 [SMILES: Cc1nc(N[C@H](C)c2cccc(C(F)F)c2F)c2cc(N(C)C3CCOCC3)ncc2n1] N⁴-{(1R)-1-[3-(difluoromethyl)-2-fluorophenyl]ethyl}-N⁶,2-dimethyl-N⁶-(oxan-4- yl)pyrido[3,4-d]pyrimidine-4,6-diamine ¹H-NMR (400 MHz, DMSO-d6) δ [ppm]: 0.866 (0.73), 0.885 (0.61), 0.904 (1.03), 0.923 (0.50), 1.231 (1.07), 1.296 (0.42), 1.511 (0.96), 1.541 (1.15), 1.610 (5.47), 1.627 (5.55), 1.775 (0.50), 1.794 (0.69), 1.805 (1.22), 1.817 (0.84), 1.825 (0.77), 1.836 (1.15), 1.847 (0.69), 1.866 (0.42), 2.289 (16.00), 2.322 (0.92), 2.326 (1.22), 2.331 (0.84), 2.456 (0.73), 2.518 (7.27), 2.522 (4.98), 2.664 (0.84), 2.668 (1.15), 2.673 (0.84), 2.942 (14.55), 3.425 (1.07), 3.454 (2.07), 3.483 (1.07), 3.946 (1.34), 3.970 (1.19), 4.930 (0.42), 4.950 (0.50), 4.959 (0.88), 4.969 (0.50), 4.989 (0.42), 5.763 (0.84), 5.782 (1.26), 5.800 (0.84), 7.101 (1.22), 7.173 (3.56), 7.237 (2.56), 7.271 (0.96), 7.290 (2.03), 7.310 (1.19), 7.373 (1.11), 7.482 (0.73), 7.499 (1.22), 7.517 (0.61), 7.638 (0.69), 7.657 (1.19), 7.675 (0.65), 8.087 (1.80), 8.406 (1.38), 8.425 (1.34), 8.655 (4.75). |
| 194 | 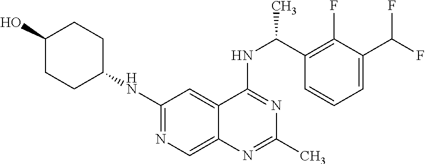 [SMILES: Cc1nc(N[C@H](C)c2cccc(C(F)F)c2F)c2cc(N[C@H]3CC[C@H](O)CC3)ncc2n1] 4-{[4-({(1R)-1-[3-(difluoromethyl)-2-fluorophenyl]ethyl}amino)-2-methylpyrido[3,4- d]pyrimidin-6-yl]amino}cyclohexan-1-ol (mixture of stereoisomers) ¹H-NMR (400 MHz, DMSO-d6) δ [ppm]: 1.106 (14.43), 1.260 (0.58), 1.284 (1.57), 1.310 (2.10), 1.319 (2.15), 1.342 (1.52), 1.595 (5.72), 1.613 (5.73), 1.876 (1.35), 1.963 (1.30), 2.265 (16.00), 2.522 (3.72), 3.436 (0.67), 3.445 (0.66), 3.564 (0.55), 4.191 (1.26), 4.578 (2.40), 4.589 (2.42), 5.725 (0.91), 5.743 (1.39), 5.762 (0.91), 6.237 (1.82), 6.259 (1.77), 6.984 (3.84), 7.098 (1.25), 7.234 (2.64), 7.265 (0.99), 7.284 (2.17), 7.303 (1.28), 7.370 (1.14), 7.476 (0.78), 7.493 (1.34), 7.511 (0.68), 7.640 (0.72), 7.659 (1.33), 7.676 (0.66), 8.249 (1.50), 8.268 (1.46), 8.529 (5.14). |
| 195 | 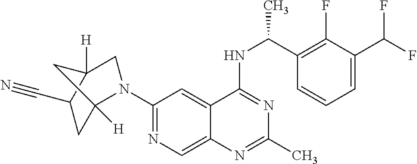 [SMILES: Cc1nc(N[C@H](C)c2cccc(C(F)F)c2F)c2cc(N3CC4CC3CC4C#N)ncc2n1] (1RS,4SR,5RS)-2-[4-({(1R)-1-[3-(difluoromethyl)-2-fluorophenyl]ethyl}amino)-2- methylpyrido[3,4-d]pyrimidin-6-yl]-2-azabicyclo[2.2.1]heptane-5-carbonitrile (mixture of stereoisomers) ¹H-NMR (400 MHz, DMSO-d6) δ [ppm]: 1.026 (0.47), 1.041 (0.47), 1.107 (0.76), 1.231 (0.64), 1.612 (5.45), 1.616 (5.80), 1.629 (5.57), 1.634 (5.57), 1.662 (1.58), 1.687 (1.82), 1.725 (0.76), 1.738 (0.88), 1.744 (0.88), 1.756 (0.76), 1.895 (1.29), 1.919 (1.11), 2.226 (0.76), 2.232 (0.76), 2.257 (1.52), 2.288 (15.53), 2.292 (16.00), 2.318 (0.47), 2.322 (0.82), 2.327 (1.11), 2.332 (0.76), 2.518 (4.04), 2.523 (2.58), 2.539 (0.70), 2.665 (0.76), 2.669 (1.05), 2.673 (0.76), 3.039 (2.05), 3.276 (0.70), 3.286 (1.11), 3.299 (1.11), 3.305 (1.17), 3.315 (1.99), 3.377 (0.47), 3.465 (0.82), 3.491 (1.82), 3.518 (1.93), 3.549 (1.05), 3.573 (0.53), 4.673 (1.70), 5.737 (0.82), 5.755 (1.64), 5.772 (1.64), 5.789 (0.82), 7.103 (1.93), 7.129 (3.99), 7.239 (3.99), 7.276 (1.47), 7.295 (3.22), 7.314 (1.88), 7.374 (1.70), 7.484 (1.05), 7.502 (1.76), 7.520 (0.88), 7.632 (0.88), 7.651 (1.58), 7.670 (0.82), 8.350 (1.29), 8.356 (1.41), 8.368 (1.35), 8.374 (1.35), 8.633 (7.79). |
| 196 | 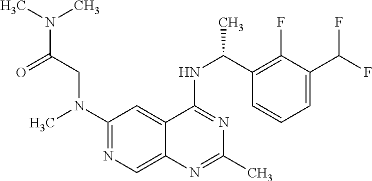 [SMILES: Cc1nc(N[C@H](C)c2cccc(C(F)F)c2F)c2cc(N(C)CC(=O)N(C)C)ncc2n1] N²-[4-({(1R)-1-[3-(difluoromethyl)-2-fluorophenyl]ethyl}amino)-2-methylpyrido[3,4- d]pyrimidin-6-yl]-N,N,N²-trimethylglycinamide ¹H-NMR (400 MHz, DMSO-d6) δ [ppm]: 0.851 (0.44), 0.860 (0.55), 0.868 (0.68), 0.886 (0.52), 0.905 (0.90), 0.924 (0.45), 1.232 (1.77), 1.256 (0.65), 1.296 (0.62), 1.348 (1.18), 1.631 (3.28), 1.648 (3.34), 2.326 (4.99), 2.518 (16.00), 2.523 (11.00), 2.665 (1.50), 2.669 (2.07), 2.673 (1.53), 2.800 (9.97), 3.022 (10.54), 3.105 (10.43), 4.518 (0.64), 4.560 (1.71), 4.595 (1.84), 4.636 (0.63), 5.794 (0.42), 5.812 (0.65), 7.109 (0.92), 7.245 (1.94), 7.273 (1.09), 7.289 (0.78), 7.309 (1.32), 7.328 (0.78), 7.381 (0.82), 7.497 (0.52), 7.515 (0.85), 7.533 (0.43), 7.660 (0.48), 7.679 (0.86), 7.698 (0.45), 8.089 (1.53), 8.591 (2.61). |
| 197 | 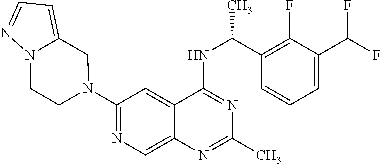 [SMILES: Cc1nc(N[C@H](C)c2cccc(C(F)F)c2F)c2cc(N3CCn4nccc4C3)ncc2n1] N-{(1R)-1-[3-(difluoromethyl)-2-fluorophenyl]ethyl}-6-(6,7-dihydropyrazolo[1,5- a]pyrazin-5(4H)-yl)-2-methylpyrido[3,4-d]pyrimidin-4-amine ¹H-NMR (400 MHz, DMSO-d6) δ [ppm]: 1.230 (0.97), 1.640 (6.40), 1.658 (6.28), 2.336 (16.00), 2.518 (3.61), 2.523 (2.54), 2.665 (0.64), 2.669 (0.87), 2.673 (0.62), 4.181 (1.18), 4.192 (2.57), 4.207 (2.42), 4.275 (2.40), 4.287 (2.81), 4.300 (1.24), 4.835 (6.96), 5.779 (0.95), 5.796 (1.43), 5.814 (0.94), 6.216 (3.42), 6.221 (3.50), 7.107 (1.51), 7.242 (3.18), 7.287 (1.16), 7.306 (2.40), 7.326 (1.41), 7.378 (1.37), 7.468 (4.77), 7.472 (4.88), 7.500 (1.02), 7.516 (1.62), 7.534 (0.78), 7.621 (3.72), 7.661 (0.86), 7.678 (1.51), 7.696 (0.81), 8.086 (0.41), 8.133 (0.46), 8.739 (6.24). |
| 198 | 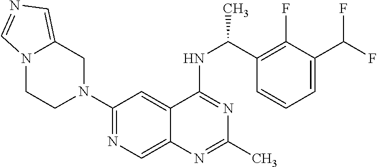 [SMILES: Cc1nc(N[C@H](C)c2cccc(C(F)F)c2F)c2cc(N3CCn4cncc4C3)ncc2n1] N-{(1R)-1-[3-(difluoromethyl)-2-fluorophenyl]ethyl}-6-(5,6-dihydroimidazo[1,5- a]pyrazin-7(8H)-yl)-2-methylpyrido[3,4-d]pyrimidin-4-amine ¹H-NMR (400 MHz, DMSO-d6) δ [ppm]: 1.631 (4.91), 1.649 (4.88), 2.317 (16.00), 2.332 (0.52), 2.518 (1.77), 2.523 (1.21), 2.539 (0.67), 2.669 (0.44), 4.067 (1.06), 4.079 (2.04), 4.094 (1.79), 4.208 (1.81), 4.223 (2.20), 4.235 (1.10), 4.789 (5.17), 5.768 (0.75), 5.786 (1.16), 5.803 (0.75), 6.853 (3.21), 6.855 (3.20), 7.105 (1.12), 7.242 (2.50), 7.280 (0.88), 7.300 (1.87), 7.319 (1.10), 7.377 (1.02), 7.492 (0.65), 7.509 (1.10), 7.528 (0.63), 7.542 (3.06), 7.639 (3.96), 7.641 (3.92), 7.649 (0.66), 7.668 (1.06), 7.686 (0.53), 8.478 (1.23), 8.496 (1.21), 8.722 (4.63). |
| 199 | 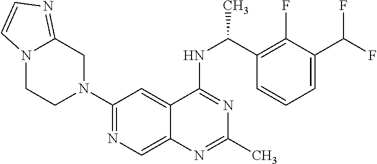 [SMILES: Cc1nc(N[C@H](C)c2cccc(C(F)F)c2F)c2cc(N3CCn4ccnc4C3)ncc2n1] N-{(1R)-1-[3-(difluoromethyl)-2-fluorophenyl]ethyl}-6-(5,6-dihydroimidazo[1,2- a]pyrazin-7(8H)-yl)-2-methylpyrido[3,4-d]pyrimidin-4-amine ¹H-NMR (400 MHz, DMSO-d6) δ [ppm]: 1.632 (5.10), 1.650 (5.07), 2.321 (16.00), 2.518 (1.57), 2.523 (1.06), 2.540 (0.56), 3.317 (0.60), 4.164 (7.15), 4.741 (3.57), 4.746 (3.60), 5.760 (0.78), 5.778 (1.22), 5.796 (0.78), 6.939 (4.93), 6.942 (4.85), 7.108 (1.23), 7.156 (4.07), 7.159 (4.07), 7.244 (2.39), 7.285 (0.89), 7.304 (1.94), 7.323 (1.15), 7.380 (1.05), 7.493 (0.70), 7.509 (1.17), 7.527 (0.58), 7.594 (3.13), 7.648 (0.64), 7.666 (1.13), 7.684 (0.57), 8.530 (1.28), 8.548 (1.24), 8.732 (4.88). |
| 200 | 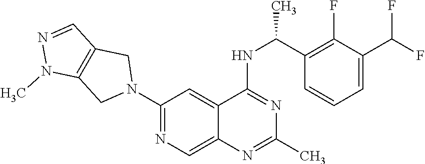 [SMILES: Cc1nc(N[C@H](C)c2cccc(C(F)F)c2F)c2cc(N3Cc4cnn(C)c4C3)ncc2n1] N-{(1R)-1-[3-(difluoromethyl)-2-fluorophenyl]ethyl}-2-methyl-6-(1-methyl-4,6- dihydropyrrolo[3,4-c]pyrazol-5(1H)-yl)pyrido[3,4-d]pyrimidin-4-amine ¹H-NMR (400 MHz, DMSO-d6) δ [ppm]: 1.631 (4.62), 1.649 (4.54), 2.282 (0.41), 2.311 (14.80), 2.322 (1.04), 2.327 (1.14), 2.332 (0.80), 2.336 (0.42), 2.518 (3.65), 2.523 (2.57), 2.665 (0.65), 2.669 (0.98), 2.673 (0.68), 3.845 (16.00), 4.559 (2.33), 4.703 (2.43), 5.782 (0.70), 5.800 (1.07), 5.818 (0.70), 7.109 (1.09), 7.195 (2.82), 7.245 (2.30), 7.286 (0.85), 7.305 (1.79), 7.328 (6.15), 7.380 (0.96), 7.494 (0.63), 7.510 (1.01), 7.528 (0.50), 7.662 (0.57), 7.681 (0.99), 7.699 (0.50), 8.445 (1.11), 8.463 (1.07), 8.697 (4.05), 10.209 (0.42). |
| 201 | 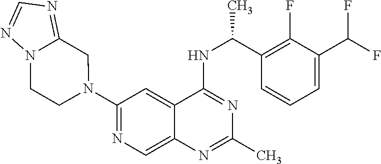 [SMILES: Cc1nc(N[C@H](C)c2cccc(C(F)F)c2F)c2cc(N3CCn4ncnc4C3)ncc2n1] N-{(1R)-1-[3-(difluoromethyl)-2-fluorophenyl]ethyl]-6-(5,6-dihydro[1,2,4]triazolo[1,5- a]pyrazin-7(8H)-yl)-2-methylpyrido[3,4-d]pyrimidin-4-amine ¹H-NMR (400 MHz, DMSO-d6) δ [ppm]: 1.229 (0.41), 1.634 (5.00), 1.652 (4.92), 2.327 (16.00), 2.518 (2.65), 2.523 (1.86), 2.539 (3.35), 2.665 (0.49), 2.669 (0.70), 2.673 (0.47), 4.263 (0.76), 4.273 (1.98), 4.287 (1.89), 4.323 (1.92), 4.336 (1.80), 4.877 (3.72), 5.760 (0.76), 5.777 (1.16), 5.795 (0.73), 7.107 (1.16), 7.243 (2.39), 7.285 (0.84), 7.304 (1.86), 7.324 (1.08), 7.379 (0.99), 7.495 (0.64), 7.513 (1.08), 7.530 (0.52), 7.650 (3.46), 7.665 (1.11), 7.685 (0.55), 8.021 (8.20), 8.506 (1.25), 8.524 (1.19), 8.748 (4.71). |
| 202 | 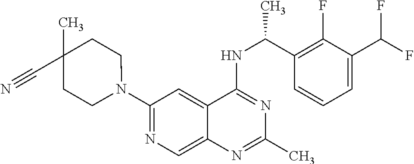 [SMILES: Cc1nc(N[C@H](C)c2cccc(C(F)F)c2F)c2cc(N3CCC(C)(C#N)CC3)ncc2n1] 1-[4-({(1R)-1-[3-(difluoromethyl)-2-fluorophenyl]ethyl}amino)-2-methylpyrido[3,4- d]pyrimidin-6-yl]-4-methylpiperidine-4-carbonitrile ¹H-NMR (400 MHz, DMSO-d6) δ [ppm]: 1.409 (3.40), 1.612 (1.60), 1.629 (1.74), 2.006 (0.47), 2.306 (4.40), 2.518 (0.86), 2.522 (0.56), 2.539 (16.00), 7.239 (0.70), 7.297 (0.55), 7.502 (1.14), 8.675 (1.34). |
| 203 | 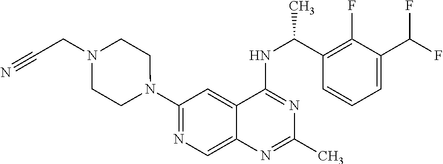 [SMILES: Cc1nc(N[C@H](C)c2cccc(C(F)F)c2F)c2cc(N3CCN(CC#N)CC3)ncc2n1] {4-[4-({(1R)-1-[3-(difluoromethyl)-2-fluorophenyl]ethyl}amino)-2-methylpyrido[3,4- d]pyrimidin-6-yl]piperazin-1-yl}acetonitrile ¹H-NMR (400 MHz, DMSO-d6) δ [ppm]: 0.860 (0.54), 0.967 (1.83), 1.107 (2.68), 1.109 (1.63), 1.144 (1.12), 1.209 (0.49), 1.610 (5.27), 1.628 (5.25), 2.309 (16.00), 2.323 (1.18), 2.327 (1.54), 2.332 (1.09), 2.336 (0.49), 2.518 (4.78), 2.523 (3.28), 2.645 (2.61), 2.658 (4.18), 2.665 (3.20), 2.669 (4.22), 3.608 (2.46), 3.622 (3.35), 3.632 (2.39), 3.841 (7.55), 5.753 (0.80), 5.770 (1.25), 5.789 (0.80), 7.103 (1.23), 7.240 (2.61), 7.278 (0.92), 7.298 (1.99), 7.317 (1.16), 7.375 (1.07), 7.480 (3.20), 7.507 (1.14), 7.524 (0.56), 7.637 (0.63), 7.655 (1.12), 7.673 (0.56), 8.439 (1.30), 8.457 (1.25), 8.676 (4.80). |
| 204 | 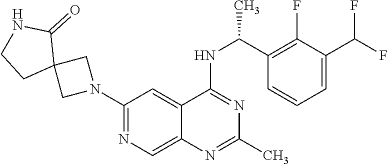 [SMILES: Cc1nc(N[C@H](C)c2cccc(C(F)F)c2F)c2cc(N3CC4(CCNC4=O)C3)ncc2n1] 2-[4-({(1R)-1-[3-(difluoromethyl)-2-fluorophenyl]ethyl}amino)-2-methylpyrido[3,4- d]pyrimidin-6-yl]-2,6-diazaspiro[3.4]octan-5-one ¹H-NMR (400 MHz, DMSO-d6) δ [ppm]: 1.107 (1.40), 1.232 (1.15), 1.600 (4.40), 1.617 (4.40), 2.303 (13.13), 2.323 (2.80), 2.327 (3.76), 2.331 (2.68), 2.444 (1.72), 2.460 (2.87), 2.518 (16.00), 2.523 (10.52), 2.540 (1.78), 2.665 (2.74), 2.669 (3.89), 2.673 (2.74), 3.210 (1.47), 3.227 (2.74), 3.243 (1.34), 3.938 (1.72), 3.943 (1.72), 3.958 (2.17), 3.963 (2.17), 4.082 (2.04), 4.094 (2.17), 4.101 (1.85), 4.113 (1.59), 5.745 (0.76), 5.760 (1.15), 5.781 (0.70), 7.103 (1.02), 7.146 (3.00), 7.239 (2.10), 7.271 (0.76), 7.291 (1.66), 7.310 (1.02), 7.375 (0.89), 7.483 (0.57), 7.500 (0.96), 7.636 (0.57), 7.655 (1.02), 7.842 (1.98), 8.415 (1.08), 8.434 (1.15), 8.640 (3.89). |
| 205 | 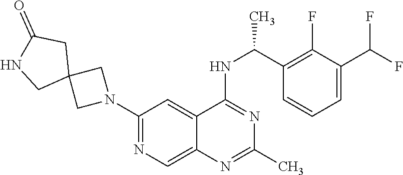 [SMILES: Cc1nc(N[C@H](C)c2cccc(C(F)F)c2F)c2cc(N3CC4(CNC(=O)C4)C3)ncc2n1] 2-[4-({(1R)-1-[3-(difluoromethyl)-2-fluorophenyl]ethyl}amino)-2-methylpyrido[3,4- d]pyrimidin-6-yl]-2,6-diazaspiro[3.4]octan-7-one ¹H-NMR (400 MHz, DMSO-d6) δ [ppm]: 1.107 (4.92), 1.596 (5.34), 1.614 (5.31), 2.299 (16.00), 2.323 (0.76), 2.327 (1.00), 2.332 (0.70), 2.518 (3.25), 2.523 (2.21), 2.553 (8.13), 2.665 (0.70), 2.669 (0.98), 2.673 (0.68), 3.532 (6.88), 3.978 (0.80), 4.002 (8.67), 4.026 (0.80), 4.190 (0.42), 5.747 (0.81), 5.765 (1.26), 5.782 (0.80), 7.099 (1.23), 7.144 (3.57), 7.235 (2.61), 7.273 (0.92), 7.293 (2.00), 7.312 (1.15), 7.371 (1.08), 7.486 (0.69), 7.502 (1.15), 7.520 (0.56), 7.630 (0.63), 7.649 (1.16), 7.668 (0.68), 7.684 (2.36), 8.421 (1.33), 8.439 (1.28), 8.631 (4.60). |
| 206 | 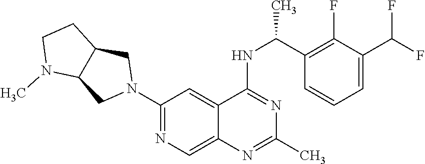 [SMILES: Cc1nc(N[C@H](C)c2cccc(C(F)F)c2F)c2cc(N3C[C@@H]4CCN(C)[C@@H]4C3)ncc2n1] N-{(1R)-1-[3-(difluoromethyl)-2-fluorophenyl]ethyl}-2-methyl-6-[(3aS,6aS)-1- methylhexahydropyrrolo[3,4-b]pyrrol-5(1H)-yl]pyrido[3,4-d]pyrimidin-4-amine ¹H-NMR (500 MHz, DMSO-d6) δ [ppm]: 1.611 (5.10), 1.624 (5.29), 1.641 (0.53), 1.660 (0.51), 2.056 (0.43), 2.069 (0.41), 2.253 (0.50), 2.271 (1.07), 2.289 (16.00), 2.302 (12.57), 2.514 (1.64), 2.518 (1.56), 2.522 (1.23), 2.923 (1.69), 3.014 (0.55), 3.028 (1.02), 3.044 (0.53), 3.294 (0.69), 3.304 (0.77), 3.315 (0.93), 3.330 (7.52), 3.352 (0.73), 3.361 (0.74), 3.374 (0.83), 3.383 (0.72), 3.631 (1.79), 3.642 (0.65), 3.679 (0.49), 5.765 (0.78), 5.778 (1.21), 5.793 (0.77), 7.087 (3.19), 7.129 (1.05), 7.238 (2.17), 7.276 (0.88), 7.292 (1.88), 7.308 (1.04), 7.347 (0.93), 7.486 (0.59), 7.500 (1.02), 7.513 (0.53), 7.644 (0.57), 7.658 (1.04), 7.673 (0.53), 8.349 (1.26), 8.363 (1.21), 8.623 (4.23). |
| 207 | 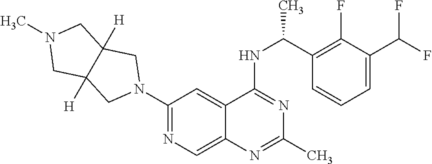 [SMILES: Cc1nc(N[C@H](C)c2cccc(C(F)F)c2F)c2cc(N3CC4CN(C)CC4C3)ncc2n1] N-{(1R)-1-[3-(difluoromethyl)-2-fluorophenyl]ethyl}-2-methyl-6-[(3aRS,6aSR)-5- methylhexahydropyrrolo[3,4-c]pyrrol-2(1H)-yl]pyrido[3,4-d]pyrimidin-4-amine (mixture of stereoisomers) ¹H-NMR (400 MHz, DMSO-d6) δ [ppm]: 1.231 (0.40), 1.604 (5.29), 1.622 (5.29), 2.238 (10.77), 2.294 (16.00), 2.460 (1.30), 2.518 (3.43), 2.522 (2.37), 2.539 (1.46), 2.564 (1.07), 2.579 (1.34), 2.601 (0.79), 2.955 (1.28), 3.330 (4.87), 3.347 (1.33), 3.354 (1.71), 3.362 (0.98), 3.373 (1.09), 3.381 (1.77), 3.594 (0.64), 3.613 (1.27), 3.620 (0.99), 3.632 (0.99), 3.639 (1.08), 3.658 (0.51), 5.758 (0.80), 5.776 (1.23), 5.794 (0.78), 7.102 (1.25), 7.138 (3.24), 7.237 (2.57), 1.Til. (0.89), 7.291 (1.94), 7.310 (1.12), 7.373 (1.08), 7.484 (0.65), 7.501 (1.11), 7.518 (0.52), 7.637 (0.60), 7.655 (1.08), 7.673 (0.54), 8.366 (1.30), 8.385 (1.24), 8.632 (4.69). |
| 208 | 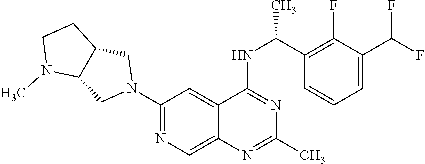 [SMILES: Cc1nc(N[C@H](C)c2cccc(C(F)F)c2F)c2cc(N3C[C@H]4CCN(C)[C@H]4C3)ncc2n1] N-{(1R)-1-[3-(difluoromethyl)-2-fluorophenyl]ethyl}-2-methyl-6-[(3aR,6aR)-1- methylhexahydropyrrolo[3,4-b]pyrrol-5(1H)-yl]pyrido[3,4-d]pyrimidin-4-amine ¹H-NMR (400 MHz, DMSO-d6) δ [ppm]: 1.609 (5.55), 1.627 (5.59), 1.650 (0.46), 1.662 (0.45), 1.672 (0.43), 2.059 (0.43), 2.075 (0.42), 2.084 (0.40), 2.289 (16.00), 2.300 (5.06), 2.307 (4.60), 2.322 (0.94), 2.326 (0.88), 2.331 (0.60), 2.394 (0.81), 2.518 (2.29), 2.522 (1.62), 2.539 (1.37), 2.664 (0.44), 2.668 (0.60), 2.673 (0.42), 2.930 (1.32), 3.015 (0.48), 3.034 (0.85), 3.054 (0.44), 3.304 (0.68), 3.318 (0.95), 3.361 (0.44), 3.377 (0.68), 3.389 (0.42), 3.396 (0.48), 3.615 (0.78), 3.635 (1.15), 3.643 (1.20), 3.661 (1.09), 5.761 (0.77), 5.780 (1.16), 5.798 (0.73), 7.088 (3.14), 7.102 (1.23), 7.238 (2.37), 7.271 (0.72), 7.290 (1.65), 7.310 (0.95), 7.373 (1.03), 7.483 (0.66), 7.499 (1.16), 7.517 (0.59), 7.637 (0.56), 7.656 (1.00), 7.674 (0.50), 8.349 (1.32), 8.368 (1.27), 8.623 (4.83). |
| 209 | 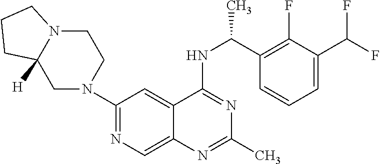 [SMILES: Cc1nc(N[C@H](C)c2cccc(C(F)F)c2F)c2cc(N3CCN4CCC[C@H]4C3)ncc2n1] N-{(1R)-1-[3-(difluoromethyl)-2-fluorophenyl]ethyl}-6-[(8aS)-hexahydropyrrolo[1,2- a]pyrazin-2(1H)-yl]-2-methylpyrido[3,4-d]pyrimidin-4-amine ¹H-NMR (400 MHz, DMSO-d6) δ [ppm]: 0.860 (0.52), 0.967 (1.48), 1.109 (1.38), 1.144 (0.94), 1.209 (0.52), 1.416 (0.50), 1.433 (0.56), 1.442 (0.54), 1.459 (0.58), 1.610 (5.18), 1.627 (5.16), 1.707 (0.69), 1.715 (0.59), 1.730 (0.79), 1.734 (0.79), 1.748 (0.50), 1.756 (0.59), 1.765 (0.44), 1.875 (0.52), 1.889 (0.56), 2.007 (0.59), 2.015 (0.52), 2.023 (0.44), 2.031 (0.54), 2.054 (0.58), 2.075 (1.30), 2.097 (1.17), 2.181 (0.46), 2.201 (0.73), 2.209 (0.77), 2.229 (0.48), 2.236 (0.40), 2.301 (16.00), 2.318 (0.48), 2.518 (5.14), 2.523 (3.59), 2.538 (1.09), 2.564 (1.07), 2.568 (1.13), 2.594 (0.82), 2.880 (0.50), 2.902 (0.77), 2.910 (0.81), 2.932 (0.44), 3.031 (0.46), 3.036 (0.48), 3.051 (0.90), 3.057 (0.86), 3.071 (0.48), 3.077 (0.42), 3.116 (0.77), 3.141 (0.73), 4.298 (0.71), 4.328 (0.67), 4.439 (0.75), 4.464 (0.73), 5.758 (0.77), 5.775 (1.21), 5.793 (0.77), 7.103 (1.19), 7.239 (2.49), 7.278 (0.84), 7.297 (1.88), 7.317 (1.07), 7.375 (1.04), 7.425 (3.07), 7.487 (0.63), 7.504 (1.09), 7.521 (0.52), 7.639 (0.58), 7.656 (1.07), 7.674 (0.52), 8.430 (1.29), 8.448 (1.23), 8.656 (4.76). |
| 210 | 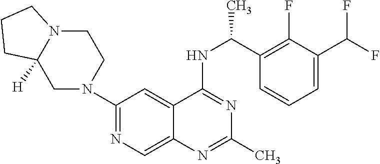 [SMILES: Cc1nc(N[C@H](C)c2cccc(C(F)F)c2F)c2cc(N3CCN4CCC[C@@H]4C3)ncc2n1] N-{(1R)-1-[3-(difluoromethyl)-2-fluorophenyl]ethyl}-6-[(8aR)-hexahydropyrrolo[1,2- a]pyrazin-2(1H)-yl]-2-methylpyrido[3,4-d]pyrimidin-4-amine ¹H-NMR (400 MHz, DMSO-d6) δ [ppm]: 0.860 (1.20), 0.967 (4.60), 1.109 (2.49), 1.144 (2.80), 1.209 (0.63), 1.225 (0.61), 1.388 (0.82), 1.414 (0.51), 1.432 (0.55), 1.441 (0.53), 1.458 (0.57), 1.610 (5.21), 1.627 (5.21), 1.706 (0.72),1.714 (0.59), 1.729 (0.80), 1.734 (0.91), 1.746 (0.51), 1.755 (0.59), 1.764 (0.42), 1.873 (0.53), 1.888 (0.55), 1.897 (0.42), 2.001 (0.59), 2.008 (0.53), 2.016 (0.44), 2.024 (0.53), 2.053 (0.51), 2.075 (1.31), 2.097 (1.20), 2.185 (0.44), 2.205 (0.74), 2.212 (0.74), 2.233 (0.51), 2.300 (16.00), 2.336 (0.42), 2.518 (4.95), 2.523 (3.60), 2.548 (1.01), 2.573 (1.05), 2.577 (1.10), 2.602 (0.82), 2.875 (0.53), 2.897 (0.76), 2.905 (0.80), 2.927 (0.44), 3.031 (0.46), 3.036 (0.48), 3.051 (0.91), 3.057 (0.86), 3.072 (0.48), 3.077 (0.42), 3.116 (0.76), 3.141 (0.74), 3.950 (0.53), 4.308 (0.70), 4.338 (0.67), 4.436 (0.76), 4.461 (0.74), 5.756 (0.78), 5.774 (1.20), 5.792 (0.76), 7.103 (1.18), 7.239 (2.49), 7.276 (0.86), 7.295 (1.90), 7.314 (1.10), 7.374 (1.03), 7.426 (3.08), 7.486 (0.65), 7.503 (1.10), 7.521 (0.53), 7.635 (0.59), 7.653 (1.08), 7.672 (0.53), 8.429 (1.31), 8.447 (1.22), 8.655 (4.76). |
| 211 | 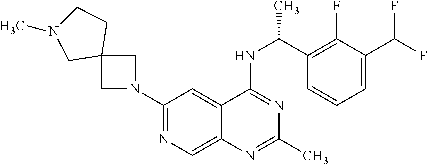 [SMILES: Cc1nc(N[C@H](C)c2cccc(C(F)F)c2F)c2cc(N3CC4(CCN(C)C4)C3)ncc2n1] N-{(1R)-1-[3-(difluoromethyl)-2-fluorophenyl]ethyl}-2-methyl-6-(6-methyl-2,6- diazaspiro[3.4]octan-2-yl)pyrido[3,4-d]pyrimidin-4-amine LC-MS ( ): Rₜ = min; MS ( ): m/z = |
| 212 | 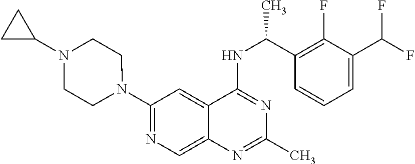 [SMILES: Cc1nc(N[C@H](C)c2cccc(C(F)F)c2F)c2cc(N3CCN(C4CC4)CC3)ncc2n1] 6-(4-cyclopropylpiperazin-1-yl)-N-{(1R)-1-[3-(difluoromethyl)-2-fluorophenyl]ethyl]-2- methylpyrido[3,4-d]pyrimidin-4-amine LC-MS ( ): Rₜ = min; MS ( ): m/z = ¹H-NMR (400 MHz, DMSO-d6) δ [ppm]: 0.369 (0.48), 0.386 (2.39), 0.394 (2.49), 0.403 (0.89), 0.445 (0.76), 0.454 (1.86), 0.460 (1.76), 0.471 (2.16), 0.488 (0.46), 1.107 (1.30), 1.605 (5.42), 1.623 (5.44), 1.664 (0.64), 1.670 (0.79), 1.679 (1.17), 1.688 (0.81), 1.695 (0.61), 2.300 (16.00), 2.322 (0.46), 2.327 (0.51), 2.522 (1.37), 2.669 (0.69), 2.684 (2.85), 2.696 (4.17), 2.708 (3.08), 3.305 (0.48), 3.514 (2.80), 3.527 (3.71), 3.539 (2.75), 5.749 (0.84), 5.767 (1.27), 5.785 (0.84), 7.100 (1.22), 7.237 (2.57), 7.276 (0.94), 7.295 (2.06), 7.314 (1.20), 7.372 (1.07), 7.436 (3.33), 7.486 (0.76), 7.503 (1.25), 7.521 (0.61), 7.634 (0.69), 7.651 (1.22), 7.669 (0.59), 8.431 (1.40), 8.449 (1.32), 8.654 (5.14). |
| 213 | 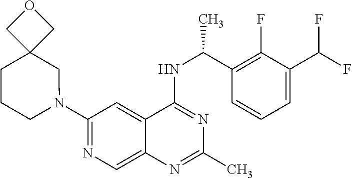 [SMILES: Cc1nc(N[C@H](C)c2cccc(C(F)F)c2F)c2cc(N3CCCC4(COC4)C3)ncc2n1] N-{(1R)-1-[3-(difluoromethyl)-2-fluorophenyl]ethyl}-2-methyl-6-(2-oxa-6- azaspiro[3.5]nonan-6-yl)pyrido[3,4-d]pyrimidin-4-amine ¹H-NMR (400 MHz, DMSO-d6) δ [ppm]: 0.852 (0.40), 0.859 (0.72), 0.967 (1.03), 1.109 (0.52), 1.144 (0.69), 1.224 (1.20), 1.231 (1.14), 1.256 (0.43), 1.348 (0.72), 1.589 (1.43), 1.620 (6.75), 1.638 (5.84), 1.850 (1.66), 1.866 (1.95), 1.880 (1.32), 2.302 (16.00), 2.464 (1.17), 2.518 (13.68), 2.522 (9.42), 3.417 (0.43), 3.434 (0.80), 3.449 (1.46), 3.469 (1.49), 3.483 (0.80), 3.502 (0.43), 3.787 (0.63), 3.818 (2.83), 3.831 (2.89), 3.862 (0.63), 4.296 (1.57), 4.310 (3.72), 4.321 (5.32), 4.325 (6.70), 4.340 (1.97), 5.759 (0.89), 5.777 (1.32), 5.795 (0.89), 7.105 (1.20), 7.241 (2.55), 7.280 (0.94), 7.299 (2.12), 7.318 (1.23), 7.377 (1.12), 7.478 (3.55), 7.505 (1.37), 7.522 (0.72), 7.639 (0.72), 7.658 (1.29), 7.676 (0.69), 8.451 (1.40), 8.469 (1.35), 8.675 (5.07). |
| 214 | 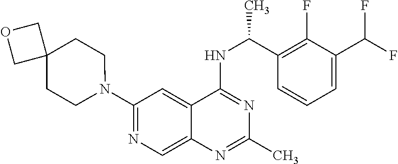 [SMILES: Cc1nc(N[C@H](C)c2cccc(C(F)F)c2F)c2cc(N3CCC4(CC3)COC4)ncc2n1] N-{(1R)-1-[3-(difluoromethyl)-2-fluorophenyl]ethyl}-2-methyl-6-(2-oxa-7- azaspiro[3.5]nonan-7-yl)pyrido[3,4-d]pyrimidin-4-amine ¹H-NMR (400 MHz, DMSO-d6) δ [ppm]: 0.859 (0.53), 0.967 (2.11), 1.107 (9.96), 1.144 (1.26), 1.224 (0.49), 1.607 (5.41), 1.625 (5.42), 1.881 (2.84), 1.895 (3.64), 1.909 (2.99), 2.295 (15.50), 2.394 (0.77), 2.518 (6.79), 2.523 (4.73), 3.528 (2.84), 3.542 (3.44), 3.556 (2.84), 4.191 (0.97), 4.386 (16.00), 5.750 (0.82), 5.768 (1.33), 5.786 (0.83), 7.102 (1.21), 7.238 (2.57), 7.277 (0.91), 7.296 (2.01), 7.315 (1.20), 7.374 (1.08), 7.450 (3.25), 7.486 (0.72), 7.504 (1.22), 7.521 (0.63), 7.630 (0.63), 7.647 (1.18), 7.666 (0.62), 8.421 (1.34), 8.439 (1.31), 8.644 (4.74). |
| 215 | 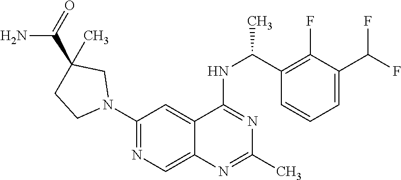 [SMILES: Cc1nc(N[C@H](C)c2cccc(C(F)F)c2F)c2cc(N3CC[C@@](C)(C(N)=O)C3)ncc2n1] (3RS)-1-[4-({(1R)-1-[3-(difluoromethyl)-2-fluorophenyl]ethyl}amino)-2-methylpyrido[3,4- d]pyrimidin-6-yl]-3-methylpyrrolidine-3-carboxamide ¹H-NMR (400 MHz, DMSO-d6) δ [ppm]: 1.103 (0.46), 1.107 (2.20), 1.230 (0.81), 1.337 (9.85), 1.603 (4.38), 1.621 (4.47), 1.907 (0.72), 1.922 (0.87), 1.937 (0.89), 1.954 (0.45), 2.285 (16.00), 2.323 (0.66), 2.327 (0.87), 2.331 (0.79), 2.349 (0.61), 2.356 (0.66), 2.368 (0.62), 2.380 (0.62), 2.387 (0.56), 2.518 (4.83), 2.523 (3.42), 2.539 (1.60), 2.665 (0.50), 2.669 (0.71), 2.673 (0.52), 3.260 (0.87), 3.275 (0.94), 3.286 (1.00), 3.300 (0.97), 3.533 (1.15), 3.551 (1.98), 3.568 (1.21), 3.868 (1.37), 3.893 (1.29), 5.760 (0.62), 5.772 (0.89), 5.778 (0.89), 5.790 (0.61), 7.029 (1.19), 7.059 (3.63), 7.103 (1.20), 7.239 (2.40), 7.271 (0.56), 7.277 (0.61), 7.290 (1.22), 7.296 (1.26), 7.309 (0.77), 7.315 (0.75), 7.375 (1.26), 7.391 (1.60), 7.480 (0.74), 7.498 (1.25), 7.515 (0.64), 7.634 (0.66), 7.653 (1.24), 7.672 (0.64), 8.399 (1.45), 8.417 (1.41), 8.615 (5.08). |
| 216 | 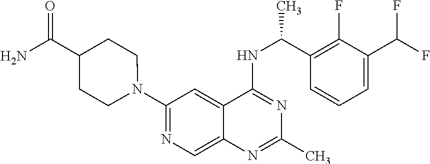 [SMILES: Cc1nc(N[C@H](C)c2cccc(C(F)F)c2F)c2cc(N3CCC(C(N)=O)CC3)ncc2n1] 1-[4-({(1R)-1-[3-(difluoromethyl)-2-fluorophenyl]ethyl}amino)-2-methylpyrido[3,4- d]pyrimidin-6-yl]piperidine-4-carboxamide ¹H-NMR (400 MHz, DMSO-d6) δ [ppm]: 1.107 (1.26), 1.584 (0.98), 1.609 (6.18), 1.626 (5.85), 1.828 (1.34), 1.855 (1.04), 2.298 (16.00), 2.323 (0.84), 2.327 (1.11), 2.331 (0.91), 2.344 (0.52), 2.364 (0.52), 2.373 (0.87), 2.518 (5.42), 2.523 (3.80), 2.540 (0.77), 2.665 (0.72), 2.669 (1.00), 2.673 (0.72), 2.843 (0.87), 2.874 (1.64), 2.904 (0.85), 4.377 (1.43), 4.410 (1.36), 5.752 (0.83), 5.770 (1.30), 5.788 (0.82), 6.825 (1.40), 7.105 (1.23), 7.240 (2.65), 7.279 (0.97), 7.298 (2.09), 7.317 (1.59), 7.327 (1.47), 7.376 (1.12), 7.438 (3.25), 7.486 (0.71), 7.503 (1.22), 7.519 (0.58), 7.635 (0.67), 7.652 (1.19), 7.671 (0.60), 8.422 (1.38), 8.441 (1.32), 8.651 (5.09). |
| 217 | 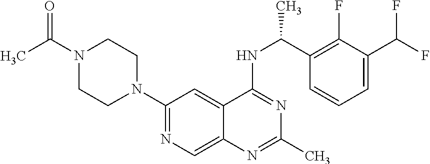 [SMILES: CC(=O)N1CCN(c2cc3c(N[C@H](C)c4cccc(C(F)F)c4F)nc(C)nc3cn2)CC1] 1-{4-[4-({(1R)-1-[3-(difluoromethyl)-2-fluorophenyl]ethyl}amino)-2-methylpyrido[3,4- d]pyrimidin-6-yl]piperazin-1-yl}ethan-1-one ¹H-NMR (400 MHz, DMSO-d6) δ [ppm]: 1.107 (10.15), 1.612 (4.86), 1.630 (4.85), 2.076 (16.00), 2.310 (15.64), 2.322 (1.07), 2.326 (1.22), 2.332 (0.83), 2.518 (4.58), 2.522 (2.92), 2.664 (0.80), 2.668 (1.08), 2.673 (0.79), 3.537 (1.36), 3.551 (1.30), 3.621 (4.61), 3.626 (4.71), 4.189 (0.90), 5.756 (0.74), 5.774 (1.18), 5.792 (0.72), 7.103 (1.15), 7.238 (2.41), 7.279 (0.84), 7.298 (1.82), 7.317 (1.04), 7.374 (1.00), 7.484 (3.13), 7.508 (1.06), 7.526 (0.52), 7.637 (0.56), 7.655 (1.03), 7.672 (0.52), 8.454 (1.21), 8.472 (1.15), 8.684 (4.54). |
| 218 | 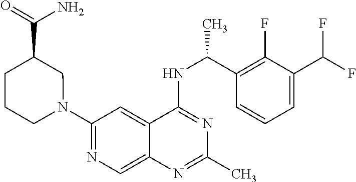 [SMILES: Cc1nc(N[C@H](C)c2cccc(C(F)F)c2F)c2cc(N3CCC[C@@H](C(N)=O)C3)ncc2n1] (3R)-1-[4-({(1R)-1-[3-(difluoromethyl)-2-fluorophenyl]ethyl}amino)-2-methylpyrido[3,4- d]pyrimidin-6-yl]piperidine-3-carboxamide ¹H-NMR (400 MHz, DMSO-d6) δ [ppm]: 1.494 (0.43), 1.525 (0.54), 1.608 (5.44), 1.626 (5.59), 1.771 (0.66), 1.779 (0.53), 1.796 (0.44), 1.803 (0.57), 1.914 (0.58), 1.938 (0.48), 2.297 (16.00), 2.322 (0.55), 2.326 (0.72), 2.332 (0.57), 2.336 (0.44), 2.345 (0.42), 2.364 (0.47), 2.374 (0.77), 2.383 (0.45), 2.518 (2.66), 2.522 (1.64), 2.539 (2.06), 2.664 (0.47), 2.669 (0.65), 2.673 (0.48), 2.787 (0.49), 2.812 (0.73), 2.817 (0.78), 2.842 (0.42), 2.863 (0.96), 2.891 (1.09), 2.895 (1.16), 2.922 (0.85), 4.358 (0.63), 4.392 (0.62), 4.406 (0.72), 4.438 (0.59), 5.758 (0.96), 5.777 (1.22), 5.794 (0.77), 6.918 (1.35), 7.103 (1.17), 7.239 (2.48), 7.279 (0.88), 7.298 (1.94), 7.317 (1.12), 7.374 (1.06), 7.428 (3.46), 7.487 (0.68), 7.504 (1.13), 7.521 (0.55), 7.633 (0.62), 7.651 (1.11), 7.669 (0.55), 8.472 (1.33), 8.490 (1.27), 8.650 (4.88). |
| 219 | 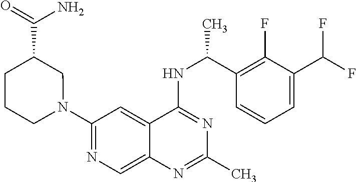 [SMILES: Cc1nc(N[C@H](C)c2cccc(C(F)F)c2F)c2cc(N3CCC[C@H](C(N)=O)C3)ncc2n1] (3S)-1-[4-({(1R)-1-[3-(difluoromethyl)-2-fluorophenyl]ethyl}amino)-2-methylpyrido[3,4- d]pyrimidin-6-yl]piperidine-3-carboxamide ¹H-NMR (400 MHz, DMSO-d6) δ [ppm]: 1.496 (0.45), 1.528 (0.57), 1.569 (0.43), 1.576 (0.41), 1.608 (5.92), 1.625 (5.73), 1.766 (0.70), 1.790 (0.46), 1.798 (0.61), 1.914 (0.61), 1.938 (0.50), 2.292 (16.00), 2.322 (0.48), 2.327 (0.70), 2.332 (0.56), 2.337 (0.55), 2.357 (0.49), 2.366 (0.81), 2.375 (0.48), 2.394 (0.42), 2.518 (2.14), 2.522 (1.35), 2.668 (0.48), 2.770 (0.51), 2.796 (0.76), 2.801 (0.79), 2.827 (0.43), 2.871 (0.91), 2.899 (1.11), 2.903 (1.12), 2.931 (0.84), 4.397 (1.09), 4.407 (0.86), 4.429 (1.04), 5.744 (0.82), 5.763 (1.27), 5.780 (0.82), 6.918 (1.43), 7.103 (1.24), 7.238 (2.59), 7.275 (0.93), 7.293 (2.02), 7.313 (1.18), 7.374 (1.11), 7.430 (4.53), 7.483 (0.72), 7.499 (1.20), 7.517 (0.59), 7.624 (0.64), 7.642 (1.17), 7.660 (0.58), 8.475 (1.38), 8.493 (1.33), 8.648 (5.19). |
| 220 | 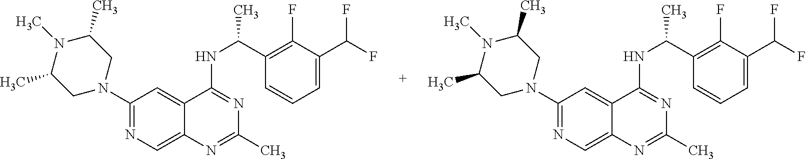 [SMILES: Cc1nc(N[C@H](C)c2cccc(C(F)F)c2F)c2cc(N3C[C@@H](C)N(C)[C@@H](C)C3)ncc2n1 ; Cc1nc(N[C@H](C)c2cccc(C(F)F)c2F)c2cc(N3C[C@@H](C)N(C)[C@@H](C)C3)ncc2n1] N-{(1R)-1-[3-(difluoromethyl)-2-fluorophenyl]ethyl}-2-methyl-6-[(cis)-3,4,5- trimethylpiperazin-1-yl]pyrido[3,4-d]pyrimidin-4-amine (mixture of stereoisomers) ¹H-NMR (400 MHz, DMSO-d6) δ [ppm]: 1.134 (10.45), 1.149 (10.80), 1.621 (5.22), 1.638 (5.22), 2.218 (15.21), 2.235 (1.44), 2.249 (1.00), 2.296 (16.00), 2.323 (0.52), 2.327 (0.68), 2.331 (0.48), 2.518 (3.88), 2.523 (2.82), 2.540 (1.42), 2.552 (1.28), 2.563 (1.14), 2.584 (1.36), 2.591 (1.26), 2.594 (1.26), 2.610 (0.91), 2.622 (0.88), 2.665 (0.43), 2.669 (0.57), 4.142 (0.78), 4.172 (1.50), 4.196 (0.66), 4.201 (0.76), 5.756 (0.79), 5.774 (1.22), 5.792 (0.78), 7.104 (1.19), 7.240 (2.51), 7.276 (0.88), 7.295 (1.93), 7.315 (1.13), 7.376 (4.15), 7.485 (0.65), 7.503 (1.13), 7.519 (0.57), 7.637 (0.60), 7.655 (1.12), 7.673 (0.57), 8.372 (1.31), 8.389 (1.27), 8.660 (4.68). |
| 221 | 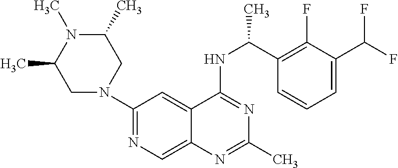 [SMILES: Cc1nc(N[C@H](C)c2cccc(C(F)F)c2F)c2cc(N3C[C@@H](C)N(C)[C@H](C)C3)ncc2n1] N-{(1R)-1-[3-(difluoromethyl)-2-fluorophenyl]ethyl}-2-methyl-6-[(3R,5R)-3,4,5- trimethylpiperazin-1-yl]pyrido[3,4-d]pyrimidin-4-amine ¹H-NMR (400 MHz, DMSO-d6) δ [ppm]: 0.860 (1.06), 0.967 (3.56), 0.995 (0.43), 1.017 (11.80), 1.033 (11.99), 1.109 (2.13), 1.144 (2.18), 1.209 (0.69), 1.225 (0.50), 1.388 (0.52), 1.614 (4.94), 1.632 (4.92), 2.265 (11.60), 2.294 (16.00), 2.518 (4.98), 2.523 (3.71), 2.660 (0.41), 2.865 (0.84), 2.874 (0.97), 2.881 (1.36), 2.890 (1.42), 2.897 (0.95), 2.905 (0.88), 3.288 (1.27), 3.304 (1.25), 3.318 (1.79), 3.587 (1.51), 3.595 (1.64), 3.617 (1.36), 3.625 (1.23), 3.950 (0.41), 5.750 (0.73), 5.768 (1.16), 5.786 (0.73), 7.104 (1.14), 7.240 (2.44), 7.278 (0.84), 7.297 (1.83), 7.316 (1.06), 7.376 (3.84), 7.485 (0.60), 7.502 (1.04), 7.520 (0.50), 7.634 (0.56), 7.653 (1.01), 7.670 (0.52), 8.378 (1.21), 8.395 (1.14), 8.644 (4.51). |
| 222 | 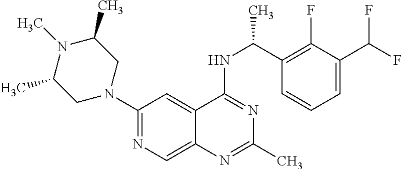 [SMILES: Cc1nc(N[C@H](C)c2cccc(C(F)F)c2F)c2cc(N3C[C@H](C)N(C)[C@@H](C)C3)ncc2n1] N-{(1R)-1-[3-(difluoromethyl)-2-fluorophenyl]ethyl]-2-methyl-6-[(3S,5S)-3,4,5- trimethylpiperazin-1-yl]pyrido[3,4-d]pyrimidin-4-amine ¹H-NMR (400 MHz, DMSO-d6) δ [ppm]: 0.859 (0.82), 0.967 (2.95), 1.014 (11.96), 1.029 (12.13), 1.109 (1.25), 1.144 (1.67), 1.208 (0.53), 1.224 (0.44), 1.388 (0.41), 1.615 (4.95), 1.632 (4.95), 2.219 (0.48), 2.265 (11.49), 2.294 (16.00), 2.518 (3.09), 2.523 (2.29), 2.865 (0.85), 2.874 (0.98), 2.881 (1.39), 2.889 (1.43), 2.897 (0.98), 2.905 (0.90), 3.273 (1.27), 3.289 (1.23), 3.303 (1.56), 3.320 (1.63), 3.608 (1.54), 3.616 (1.66), 3.638 (1.40), 3.646 (1.29), 5.755 (0.75), 5.773(1.17),5.791(0.75),7.103(1.14),7.239 (2.47),7.278 (0.85), 7.297 (1.89), 7.316 (1.08),7.375(1.40),7.382(3.02),7.487(0.63), 7.504(1.08), 7.521 (0.51), 7.636 (0.58), 7.654 (1.07), 7.673 (0.53), 8.375 (1.25), 8.393 (1.18), 8.642 (4.57). |
| 223 | 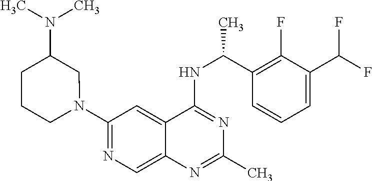 [SMILES: Cc1nc(N[C@H](C)c2cccc(C(F)F)c2F)c2cc(N3CCCC(N(C)C)C3)ncc2n1] N-{(1R)-1-[3-(difluoromethyl)-2-fluorophenyl]ethyl}-6-[3-(dimethylamino)piperidin-1-yl]- 2-methylpyrido[3,4-d]pyrimidin-4-amine (mixture of stereoisomers) ¹H-NMR (400 MHz, DMSO-d6) δ [ppm]: 0.859 (0.53), 0.967 (2.02), 1.109 (1.06), 1.144 (1.14), 1.464 (0.40), 1.607 (3.47), 1.625 (3.48), 1.831 (0.43), 2.270 (16.00), 2.289 (6.29), 2.294 (6.22), 2.323 (0.84), 2.327 (1.06), 2.331 (0.76), 2.518 (5.57), 2.523 (3.97), 2.665 (0.60), 2.669 (0.83), 2.674 (0.57), 2.779 (0.44), 2.804 (0.83), 2.829 (0.56), 2.835 (0.55), 3.129 (0.89), 5.762 (0.49), 5.773 (0.49), 7.103 (0.64), 7.238 (1.29), 7.275 (0.57), 7.294 (1.27), 7.314 (0.75), 7.374 (0.60), 7.405 (1.93), 7.485 (0.43), 7.500 (0.73), 7.646 (0.65), 8.435 (0.79), 8.453 (0.78), 8.639 (3.03). |
| 224 | 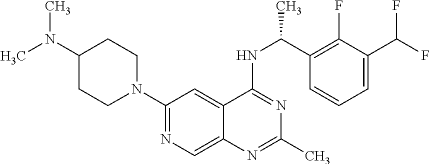 [SMILES: Cc1nc(N[C@H](C)c2cccc(C(F)F)c2F)c2cc(N3CCC(N(C)C)CC3)ncc2n1] N-{(1R)-1-[3-(difluoromethyl)-2-fluorophenyl]ethyl]-6-[4-(dimethylamino)piperidin-1-yl]- 2-methylpyrido[3,4-d]pyrimidin-4-amine ¹H-NMR (400 MHz, DMSO-d6) δ [ppm]: 0.967 (0.47), 1.413 (0.45), 1.442 (0.49), 1.607 (2.84), 1.625 (2.84), 1.865 (0.60), 1.895 (0.52), 2.204 (16.00), 2.295 (8.63), 2.323 (0.60), 2.327 (0.68), 2.332 (0.74), 2.518 (1.52), 2.523 (1.05), 2.669 (0.47), 2.840 (0.42), 2.872 (0.74), 2.903 (0.41), 4.368 (0.51), 4.395 (0.49), 5.750 (0.43), 5.768 (0.66), 5.786 (0.41), 7.103 (0.66), 7.238 (1.42), 7.276 (0.48), 7.296 (1.06), 7.315 (0.61), 7.374 (0.59), 7.435 (1.63), 7.503 (0.60), 7.652 (0.58), 8.416 (0.69), 8.433 (0.65), 8.642 (2.50). |
| 225 | 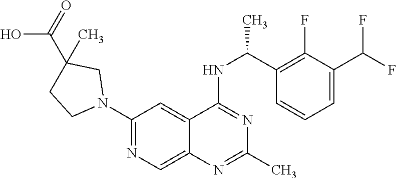 [SMILES: Cc1nc(N[C@H](C)c2cccc(C(F)F)c2F)c2cc(N3CCC(C)(C(=O)O)C3)ncc2n1] 1-[4-({(1R)-1-[3-(difluoromethyl)-2-fluorophenyl]ethyl}amino)-2-methylpyrido[3,4- d]pyrimidin-6-yl]-3-methylpyrrolidine-3-carboxylic acid (mixture of stereoisomers) ¹H-NMR (400 MHz, DMSO-d6) δ [ppm]: 0.929 (0.42), 0.946 (0.87), 0.964 (0.47), 1.107 (0.42), 1.232 (1.13), 1.368 (9.69), 1.605 (4.60), 1.622 (4.65), 1.892 (0.66), 1.905 (1.29), 1.922 (0.68), 2.287 (14.08), 2.336 (0.50), 2.401 (0.68), 2.416 (0.68), 2.432 (0.66), 2.449 (0.47), 2.518 (6.41), 2.523 (4.15), 2.540 (16.00), 3.275 (1.71), 3.540 (0.53), 3.558 (1.00), 3.575 (1.23), 3.594 (0.89), 3.609 (0.42), 3.891 (0.92), 3.903 (0.95), 3.917 (0.89), 3.929 (0.89), 5.753 (0.47), 5.759 (0.71), 5.771 (0.76), 5.778 (0.76), 5.789 (0.53), 5.796 (0.50), 7.072 (3.28), 7.103 (1.16), 7.239 (2.36), 7.275 (0.74), 7.293 (1.60), 7.312 (0.95), 7.375 (1.00), 7.481 (0.71), 7.498 (1.18), 7.516 (0.60), 7.637 (0.66), 7.655 (1.18), 7.674 (0.58), 8.386 (1.23), 8.404 (1.21), 8.623 (4.91). |
| 226 | 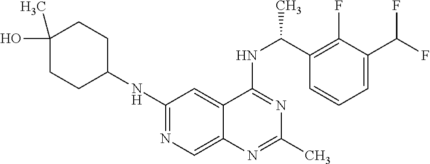 [SMILES: Cc1nc(N[C@H](C)c2cccc(C(F)F)c2F)c2cc(NC3CCC(C)(O)CC3)ncc2n1] 4-{[4-({(1R)-1-[3-(difluoromethyl)-2-fluorophenyl]ethyl}amino)-2-methylpyrido[3,4- d]pyrimidin-6-yl]amino}-1-methylcyclohexan-1-ol (mixture of stereoisomers) ¹H-NMR (400 MHz, DMSO-d6) δ [ppm]: 1.107 (11.02), 1.156 (13.59), 1.463 (2.17), 1.593 (6.19), 1.610 (6.83), 1.889 (1.37), 2.266 (16.00), 2.323 (2.01), 2.327 (2.81), 2.331 (2.09), 2.522 (8.84), 2.664 (2.01), 2.668 (2.81), 2.673 (2.09), 3.292 (0.48), 3.365 (1.37), 3.385 (0.80), 3.695 (0.64), 4.193 (1.05), 4.253 (5.23), 5.727 (0.88), 5.744 (1.37), 5.761 (0.88), 6.277 (1.77), 6.298 (1.69), 7.004 (3.78), 7.098 (1.21), 7.234 (2.73), 7.265 (0.96), 7.284 (2.17), 7.304 (1.21), 7.369 (1.13), 7.477 (0.80), 7.493 (1.29), 7.512 (0.72), 7.640 (0.72), 7.660 (1.37), 7.677 (0.72), 8.261 (1.45), 8.280 (1.45), 8.531 (5.15). |
| 227 | 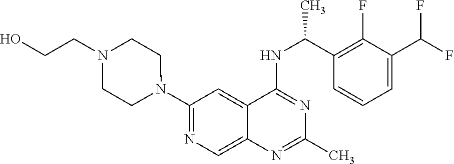 [SMILES: Cc1nc(N[C@H](C)c2cccc(C(F)F)c2F)c2cc(N3CCN(CCO)CC3)ncc2n1] 2-{4-[4-({(1R)-1-[3-(difluoromethyl)-2-fluorophenyl]ethyl}amino)-2-methylpyrido[3,4- d]pyrimidin-6-yl]piperazin-1-yl}ethan-1-ol ¹H-NMR (400 MHz, DMSO-d6) δ [ppm]: 0.967 (1.32), 1.107 (16.00), 1.143 (0.80), 1.224 (0.43), 1.605 (4.68), 1.623 (4.66), 2.301 (12.93), 2.322 (0.62), 2.326 (0.72), 2.331 (0.54), 2.447 (1.86), 2.463 (3.98), 2.479 (2.85), 2.522 (2.55), 2.539 (0.92), 2.570 (2.51), 2.582 (3.51), 2.594 (2.64), 2.665 (0.42), 2.669 (0.58), 2.673 (0.42), 3.542 (3.48), 3.558 (5.69), 3.571 (3.78), 3.587 (1.16), 4.191 (1.51), 4.455 (1.29), 4.469 (2.75), 4.482 (1.22), 5.751 (0.75), 5.769 (1.15), 5.786 (0.74), 7.103 (1.04), 7.238 (2.21), 7.276 (0.80), 7.295 (1.77), 7.315 (1.05), 7.374 (0.95), 7.432 (2.79), 7.486 (0.64), 7.504 (1.08), 7.521 (0.55), 7.633 (0.61), 7.652 (1.08), 7.669 (0.54), 8.429 (1.18), 8.446 (1.14), 8.656 (4.17). |
| 228 | 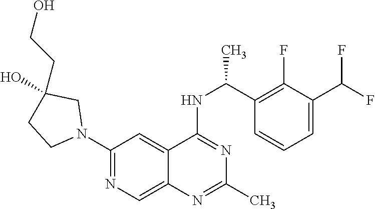 [SMILES: Cc1nc(N[C@H](C)c2cccc(C(F)F)c2F)c2cc(N3CC[C@@](O)(CCO)C3)ncc2n1] 1-[4-({(1R)-1-[3-(difluoromethyl)-2-fluorophenyl]ethyl}amino)-2-methylpyrido[3,4- d]pyrimidin-6-yl]-3-(2-hydroxyethyl)pyrrolidin-3-ol (mixture of stereoisomers) ¹H-NMR (400 MHz, DMSO-d6) δ [ppm]: 1.026 (0.44), 1.042 (0.44), 1.607 (5.93), 1.625 (5.99), 1.836 (1.20), 1.854 (2.18), 1.859 (2.23), 1.876 (1.25), 1.950 (0.87), 1.970 (1.96), 1.989 (1.63), 2.281 (16.00), 2.323 (0.71), 2.327 (0.93), 2.331 (0.65), 2.523 (2.94), 2.540 (0.65), 2.665 (0.65), 2.669 (0.93), 2.673 (0.65), 3.301 (0.54), 3.312 (0.98), 3.370 (2.18), 3.385 (1.14), 3.399 (1.58), 3.413 (1.31), 3.487 (0.98), 3.501 (1.09), 3.515 (1.14), 3.530 (1.14), 3.559 (0.98), 3.575 (0.93), 3.585 (0.82), 3.611 (0.60), 3.643 (1.09), 3.661 (2.45), 3.673 (2.45), 3.690 (1.03), 4.479 (1.36), 4.491 (2.88), 4.504 (1.31), 4.745 (2.94), 4.752 (2.94), 5.762 (0.82), 5.779 (1.25), 5.797 (0.82), 7.009 (3.21), 7.101 (1.36), 7.236 (2.83), 7.271 (0.87), 7.290 (1.96), 7.310 (1.14), 7.372 (1.20), 7.480 (0.87), 7.497 (1.47), 7.515 (0.71), 7.631 (0.76), 7.649 (1.41), 7.668 (0.71), 8.365 (1.58), 8.384 (1.47), 8.610 (5.44). |
| 229 | 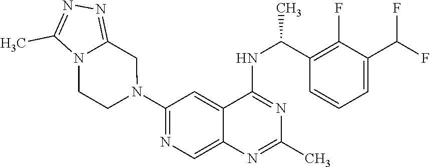 [SMILES: Cc1nc(N[C@H](C)c2cccc(C(F)F)c2F)c2cc(N3CCn4c(C)nnc4C3)ncc2n1] N-{(1R)-1-[3-(difluoromethyl)-2-fluorophenyl]ethyl}-2-methyl-6-(3-methyl-5,6- dihydro[1,2,4]triazolo[4,3-a]pyrazin-7(8H)-yl)pyrido[3,4-d]pyrimidin-4-amine ¹H-NMR (400 MHz, DMSO-d6) δ [ppm]: 1.231 (0.95), 1.633 (4.68), 1.651 (4.63), 2.263 (0.92), 2.323 (16.00), 2.334 (16.00), 2.518 (5.21), 2.523 (3.76), 2.539 (0.92), 2.660 (0.41), 2.665 (0.97), 2.669 (1.38), 2.673 (0.97), 2.678 (0.44), 4.055 (0.81), 4.069 (2.01), 4.082 (1.59), 4.160 (1.54), 4.175 (1.91), 4.187 (0.92), 4.875 (3.20), 4.880 (3.23), 5.756 (0.71), 5.774 (1.11), 5.792 (0.69), 7.108 (1.06), 7.244 (2.33), 7.287 (0.83), 7.305 (1.84), 7.324 (1.04), 7.380 (0.97), 7.496 (0.62), 7.513 (1.06), 7.530 (0.53), 7.646 (3.25), 7.663 (1.01), 7.681 (0.51), 8.505 (1.15), 8.523 (1.08), 8.738 (4.31). |
| 230 | 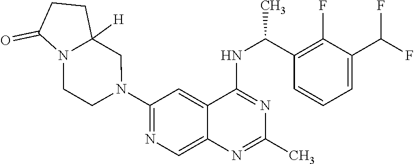 [SMILES: Cc1nc(N[C@H](C)c2cccc(C(F)F)c2F)c2cc(N3CCN4C(=O)CCC4C3)ncc2n1] 2-[4-({(1R)-1-[3-(difluoromethyl)-2-fluorophenyl]ethyl}amino)-2-methylpyrido[3,4- d]pyrimidin-6-yl]hexahydropyrrolo[1,2-a]pyrazin-6(2H)-one (mixture of stereoisomers) ¹H-NMR (500 MHz, DMSO-d6) δ [ppm]: 1.191 (0.57), 1.203 (0.61), 1.231 (0.49), 1.471 (0.41), 1.619 (5.64), 1.633 (5.64), 1.658 (0.48), 1.674 (0.50), 1.678 (0.55), 1.684 (0.60), 1.698 (0.57), 2.100 (0.91), 2.192 (0.46), 2.197 (0.81), 2.207 (0.68), 2.215 (0.64), 2.240 (0.49), 2.286 (1.08), 2.295 (1.44), 2.300 (1.64), 2.308 (14.38), 2.324 (1.04), 2.333 (0.63), 2.357 (0.60), 2.361 (0.85), 2.365 (0.65), 2.514 (2.46), 2.518 (2.42), 2.522 (1.96), 2.539 (16.00), 2.595 (0.55), 2.604 (0.59), 2.617 (0.66), 2.620 (0.68), 2.630 (0.99), 2.635 (0.90), 2.639 (0.72), 2.642 (0.82), 2.651 (0.57), 2.794 (0.74), 2.800 (0.78), 2.819 (0.51), 2.825 (0.50), 2.912 (0.60), 3.651 (0.59), 3.660 (0.59), 3.665 (0.58), 3.674 (0.56), 3.935 (0.79), 3.939 (0.79), 3.960 (0.75), 4.416 (0.60), 4.440 (0.58), 4.566 (0.69), 4.592 (0.66), 5.761 (0.84), 5.775 (1.29), 5.789 (0.82), 7.131 (1.21), 7.239 (2.56), 7.284 (0.88), 7.300 (1.82), 7.315 (1.01), 7.348 (1.07), 7.494 (0.74), 7.508 (1.23), 7.520 (3.84), 7.641 (0.61), 7.656 (1.07), 7.670 (0.58), 8.434 (1.26), 8.448 (1.18), 8.681 (4.94). |
| 231 | 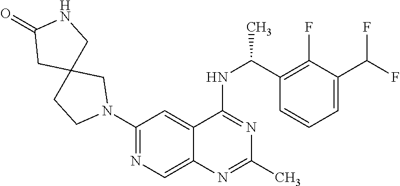 [SMILES: Cc1nc(N[C@H](C)c2cccc(C(F)F)c2F)c2cc(N3CCC4(CNC(=O)C4)C3)ncc2n1] (5RS)-7-[4-({(1R)-1-[3-(difluoromethyl)-2-fluorophenyl]ethyl}amino)-2-methylpyrido[3,4- d]pyrimidin-6-yl]-2,7-diazaspiro[4.4]nonan-3-one (mixture of stereoisomers) ¹H-NMR (400 MHz, DMSO-d6) δ [ppm]: 1.107 (6.79), 1.230 (0.49), 1.603 (4.68), 1.620 (4.70), 2.033 (0.92), 2.053 (1.86), 2.069 (1.17), 2.289 (16.00), 2.294 (7.47), 2.322 (0.49), 2.327 (0.61), 2.332 (0.45), 2.518 (2.42), 2.522 (1.47), 2.539 (0.48), 2.669 (0.55), 3.269 (5.06), 3.444 (0.59), 3.457 (0.50), 3.469 (1.04), 3.482 (1.11), 3.508 (1.11), 3.518 (1.07), 3.533 (0.50), 3.544 (0.65), 3.569 (0.84), 3.588 (1.60), 3.603 (0.82), 4.190 (0.58), 5.761 (0.69), 5.779 (1.07), 5.797 (0.69), 7.072 (2.91), 7.101 (1.07), 7.237 (2.28), 7.275 (0.82), 7.294 (1.79), 7.313 (1.04), 7.373 (0.94), 7.483 (0.61), 7.501 (1.04), 7.518 (0.52), 7.635 (0.56), 7.653 (1.02), 7.672 (0.52), 7.711 (2.16), 8.380 (1.18), 8.398 (1.15), 8.626 (4.37). |
| 232 | 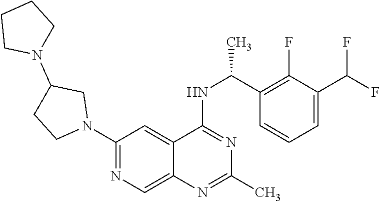 [SMILES: Cc1nc(N[C@H](C)c2cccc(C(F)F)c2F)c2cc(N3CCC(N4CCCC4)C3)ncc2n1] 6-[[1,3′-bipyrrolidin]-1′-yl]-N-{(1R)-1-[3-(difluoromethyl)-2-fluorophenyl]ethyl]-2- methylpyrido[3,4-d]pyrimidin-4-amine (mixture of stereoisomers) ¹H-NMR (400 MHz, DMSO-d6) δ [ppm]: 1.610 (5.63), 1.628 (5.71), 1.719 (4.90), 1.929 (0.57), 1.950 (0.69), 1.959 (0.78), 1.981 (0.75), 2.182 (0.55), 2.190 (0.59), 2.205 (0.60), 2.285 (16.00), 2.518 (5.03), 2.523 (4.04), 2.543 (3.22), 2.557 (2.99), 2.867 (0.60), 2.883 (0.77), 2.904 (0.61), 3.253 (0.93), 3.271 (0.97), 3.278 (1.13), 3.297 (0.95), 3.421 (0.77), 3.440 (0.78), 3.462 (0.44), 3.604 (0.48), 3.611 (0.56), 3.629 (0.82), 3.650 (0.44), 3.721 (0.84), 3.738 (0.95), 3.747 (0.90), 3.763 (0.74), 5.766 (0.85), 5.784 (1.31), 5.802 (0.87), 7.043 (3.51), 7.101 (1.27), 7.238 (2.70), 7.269 (0.98), 7.288 (2.10), 7.307 (1.23), 7.373 (1.11), 7.481 (0.74), 7.497 (1.28), 7.516 (0.66), 7.634 (0.69), 7.652 (1.25), 7.669 (0.65), 8.341 (1.42), 8.360 (1.38), 8.623 (4.91). |
| 233 | 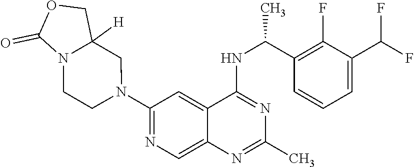 [SMILES: Cc1nc(N[C@H](C)c2cccc(C(F)F)c2F)c2cc(N3CCN4C(=O)OCC4C3)ncc2n1] 7-[4-({(1R)-1-[3-(difluoromethyl)-2-fluorophenyl]ethyl}amino)-2-methylpyrido[3,4- d]pyrimidin-6-yl]hexahydro-3H-[1,3]oxazolo[3,4-a]pyrazin-3-one (mixture of stereoisomers) ¹H-NMR (400 MHz, DMSO-d6) δ [ppm]: 0.937 (0.60), 0.961 (0.71), 1.231 (0.87), 1.615 (7.75), 1.633 (7.66), 2.311 (16.00), 2.322 (1.47), 2.327 (1.26), 2.332 (0.87), 2.336 (0.44), 2.518 (3.42), 2.523 (2.39), 2.539 (1.17), 2.664 (0.73), 2.669 (1.03), 2.673 (0.74), 2.779 (0.85), 2.783 (0.88), 2.811 (1.73), 2.838 (0.92), 2.843 (0.88), 2.891 (0.51), 2.895 (0.46), 2.921 (0.97), 2.944 (0.60), 2.949 (0.69), 3.128 (0.60), 3.140 (0.51), 3.150 (0.69), 3.161 (0.92), 3.172 (0.64), 3.181 (0.44), 3.194 (0.48), 3.717 (1.17), 3.723 (1.17), 3.749 (1.06), 3.756 (0.97), 3.922 (0.42), 3.936 (0.65), 3.946 (0.73), 3.957 (0.76), 3.963 (0.65), 3.970 (0.60), 3.984 (0.51), 4.055 (1.73), 4.069 (1.40), 4.077 (1.89), 4.091 (1.49), 4.342 (0.94), 4.372 (0.88), 4.454 (1.95), 4.475 (3.42), 4.497 (1.61), 4.594 (0.96), 4.601 (0.96), 4.625 (0.94), 4.634 (0.88), 5.756 (0.90), 5.774 (1.38), 5.791 (0.90), 7.103 (1.77), 7.239 (3.70), 7.280 (1.31), 7.299 (2.83), 7.318 (1.65), 7.374 (1.56), 7.492 (1.01), 7.509 (1.70), 7.534 (4.67), 7.636 (0.90), 7.655 (1.61), 7.672 (0.81), 8.433 (1.59), 8.451 (1.50), 8.685 (7.13). |
| 234 | 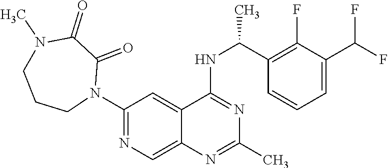 [SMILES: Cc1nc(N[C@H](C)c2cccc(C(F)F)c2F)c2cc(N3CCCN(C)C(=O)C3=O)ncc2n1] 1-[4-({(1R)-1-[3-(difluoromethyl)-2-fluorophenyl]ethyl}amino)-2-methylpyrido[3,4- d]pyrimidin-6-yl]-4-methyl-1,4-diazepane-2,3-dione Isolated as a by-product when using 4-aminopyrrolidin-2-one hydrochloride ¹H-NMR (400 MHz, DMSO-d6) δ [ppm]: 0.835 (0.86), 0.849 (1.20), 0.868 (0.52), 1.232 (2.49), 1.348 (0.43), 1.602 (5.25), 1.620 (5.42), 1.960 (0.52), 1.967 (0.60), 1.971 (0.77), 1.988 (0.95), 2.042 (9.20), 2.081 (1.20), 2.106 (0.60), 2.125 (1.29), 2.147 (4.99), 2.207 (0.52), 2.228 (0.69), 2.283 (1.89), 2.295 (16.00), 2.332 (3.27), 2.336 (1.72), 2.518 (11.78), 2.523 (7.40), 2.673 (2.67), 2.678 (1.12), 2.787 (3.27), 2.791 (3.35), 2.932 (6.11), 2.935 (6.19), 3.011 (0.69), 3.291 (0.43), 3.371 (0.52), 3.413 (0.52), 3.434 (0.86), 3.459 (0.95), 3.478 (0.86), 3.594 (0.69), 3.620 (0.69), 3.717 (0.86), 3.733 (0.95), 5.205 (0.43), 5.226 (0.69), 5.245 (0.43), 5.761 (0.69), 5.777 (1.12), 5.794 (0.69), 7.101 (3.10), 7.122 (1.46), 7.237 (3.87), 7.275 (1.38), 7.294 (2.92), 7.313 (1.72), 7.373 (1.72), 7.485 (0.95), 7.501 (1.63), 7.520 (0.86), 7.631 (0.86), 7.649 (1.63), 7.667 (0.86), 8.363 (1.29), 8.380 (1.20), 8.616 (0.43), 8.644 (2.92), 8.652 (2.06). |
| 235 | 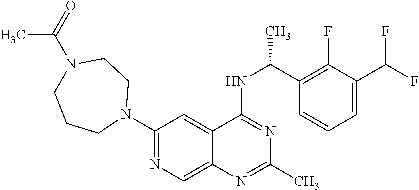 [SMILES: CC(=O)N1CCCN(c2cc3c(N[C@H](C)c4cccc(C(F)F)c4F)nc(C)nc3cn2)CC1] 1-{4-[4-({(1R)-1-[3-(difluoromethyl)-2-fluorophenyl]ethyl}amino)-2-methylpyrido[3,4- d]pyrimidin-6-yl]-1,4-diazepan-1-yl}ethan-1-one ¹H-NMR (400 MHz, DMSO-d6) δ [ppm]: 1.617 (7.40), 1.635 (7.36), 1.839 (13.53), 1.908 (0.76), 1.922 (1.03), 1.937 (0.76), 1.993 (11.31), 2.284 (14.46), 2.287 (16.00), 2.318 (0.72), 2.460 (1.00), 2.518 (8.32), 2.523 (6.01), 3.358 (0.84), 3.371 (0.80), 3.384 (0.82), 3.397 (1.32), 3.413 (1.61), 3.427 (0.97), 3.637 (0.47), 3.651 (0.93), 3.673 (1.20), 3.686 (2.38), 3.699 (2.51), 3.733 (1.00), 3.745 (1.39), 3.758 (1.06), 3.779 (0.87), 3.793 (0.59), 3.826 (1.48), 3.840 (1.06), 3.853 (0.43), 3.875 (0.47), 3.891 (0.68), 3.953 (0.72), 5.758 (1.15), 5.776 (1.73), 5.794 (1.10), 7.103 (1.30), 7.238 (4.71), 7.256 (2.43), 7.279 (1.34), 7.298 (2.90), 7.317 (1.68), 7.375 (1.21), 7.485 (1.00), 7.502 (1.68), 7.520 (0.84), 7.626 (0.69), 7.644 (1.23), 7.663 (0.62), 8.394 (1.26), 8.412 (1.27), 8.629 (3.48), 8.636 (3.75). |
| 236 | 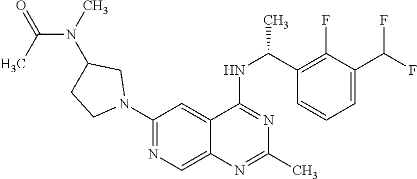 [SMILES: CC(=O)N(C)C1CCN(c2cc3c(N[C@H](C)c4cccc(C(F)F)c4F)nc(C)nc3cn2)C1] N-{(3RS)-1-[4-({(1R)-1-[3-(difluoromethyl)-2-fluorophenyl]ethyl}amino)-2- methylpyrido[3,4-d]pyrimidin-6-yl]pyrrolidin-3-yl}-N-methylacetamide (mixture of stereoisomers) ¹H-NMR (400 MHz, DMSO-d6) δ [ppm]: 1.107 (3.50), 1.231 (0.49), 1.603 (5.35), 1.620 (5.55), 1.988 (0.49), 2.042 (9.42), 2.081 (0.62), 2.105 (0.66), 2.125 (1.44), 2.147 (5.51), 2.206 (0.66), 2.228 (0.82), 2.295 (16.00), 2.323 (1.11), 2.327 (1.40), 2.331 (1.19), 2.518 (6.42), 2.523 (4.07), 2.540 (9.99), 2.665 (0.90), 2.669 (1.32), 2.673 (0.90), 2.787 (3.70), 2.791 (3.83), 2.934 (6.83), 3.300 (0.53), 3.379 (0.66), 3.392 (0.58), 3.414 (0.62), 3.434 (0.95), 3.459 (1.15), 3.479 (0.95), 3.594 (0.74), 3.620 (0.66), 3.716 (0.99), 3.733 (1.19), 5.207 (0.49), 5.227 (0.70), 5.246 (0.49), 5.759 (0.82), 5.777 (1.11), 5.795 (0.74), 7.101 (3.17), 7.123 (1.65), 7.237 (3.41), 7.275 (1.28), 7.294 (2.84), 7.313 (1.65), 7.373 (1.52), 7.486 (0.99), 7.502 (1.69), 7.520 (0.86), 7.630 (0.90), 7.650 (1.69), 7.668 (0.86), 8.366 (1.36), 8.383 (1.36), 8.644 (3.17), 8.652 (2.34). |
| 237 | 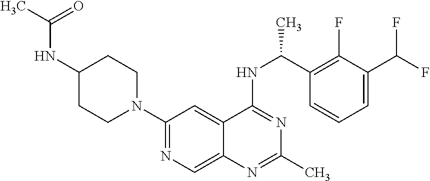 [SMILES: CC(=O)NC1CCN(c2cc3c(N[C@H](C)c4cccc(C(F)F)c4F)nc(C)nc3cn2)CC1] N-{1-[4-({(1R)-1-[3-(difluoromethyl)-2-fluorophenyl]ethyl}amino)-2-methylpyrido[3,4- d]pyrimidin-6-yl]piperidin-4-yl}acetamide ¹H-NMR (400 MHz, DMSO-d6) δ [ppm]: 1.107 (2.36), 1.402 (0.76), 1.409 (0.83), 1.432 (0.87), 1.438 (0.83), 1.459 (0.40), 1.606 (4.73), 1.624 (4.75), 1.799 (16.00), 1.847 (1.06), 1.871 (0.91), 2.299 (14.31), 2.322 (0.55), 2.327 (0.65), 2.332 (0.48), 2.518 (2.53), 2.523 (1.52), 2.539 (0.57), 2.664 (0.44), 2.669 (0.63), 2.673 (0.46), 2.983 (0.71), 3.016 (1.18), 3.046 (0.70), 3.823 (0.48), 3.834 (0.41), 3.843 (0.46), 4.282 (1.14), 4.315 (1.08), 4.579 (0.63), 4.593 (1.08), 4.610 (0.87), 4.662 (0.83), 4.677 (0.63), 4.684 (0.84), 4.698 (0.58), 5.754 (0.73), 5.772 (1.11), 5.790 (0.72), 7.102 (1.07), 7.238 (2.32), 7.279 (0.82), 7.298 (1.79), 7.317 (1.04), 7.374 (0.94), 7.450 (2.83), 7.489 (0.62), 7.505 (1.05), 7.523 (0.51), 7.632 (0.57), 7.651 (1.04), 7.668 (0.52), 7.839 (1.28), 7.858 (1.25), 8.421 (1.20), 8.439 (1.15), 8.653 (4.58). |
| 238 | 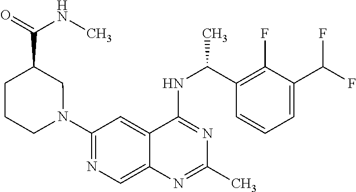 [SMILES: CNC(=O)[C@@H]1CCCN(c2cc3c(N[C@H](C)c4cccc(C(F)F)c4F)nc(C)nc3cn2)C1] (3RS)-1-[4-({(1R)-1-[3-(difluoromethyl)-2-fluorophenyl]ethyl}amino)-2-methylpyrido[3,4- d]pyrimidin-6-yl]-N-methylpiperidine-3-carboxamide (mixture of stereoisomers) ¹H-NMR (400 MHz, DMSO-d6) δ [ppm]: 1.030 (0.95), 1.094 (2.38), 1.231 (0.65), 1.493 (0.44), 1.524 (0.55), 1.605 (4.22), 1.623 (4.79), 1.655 (0.57), 1.769 (0.55), 1.803 (0.46), 1.862 (0.59), 1.907 (0.53), 2.291 (6.76), 2.296 (6.76), 2.322 (1.01), 2.326 (1.31), 2.331 (1.06), 2.363 (0.59), 2.371 (0.65), 2.522 (4.94), 2.539 (16.00), 2.592 (5.78), 2.603 (5.76), 2.665 (0.86), 2.669 (1.16), 2.673 (0.87), 2.803 (0.48), 2.870 (0.44), 2.883 (0.44), 2.901 (0.57), 2.912 (1.01), 2.930 (0.42), 4.406 (0.91), 4.425 (0.65), 5.744 (0.42), 5.759 (0.86), 5.772 (0.63), 7.103 (0.89), 7.238 (1.88), 7.277 (0.53), 7.295 (1.14), 7.313 (0.70), 7.374 (0.80), 7.424 (2.45), 7.485 (0.55), 7.502 (0.93), 7.519 (0.51), 7.644 (0.74), 7.899 (0.67), 7.909 (0.89), 7.918 (0.68), 8.463 (0.95), 8.481 (0.95), 8.646 (3.40). |
| 239 | 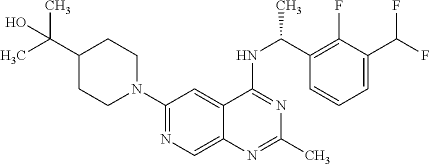 [SMILES: Cc1nc(N[C@H](C)c2cccc(C(F)F)c2F)c2cc(N3CCC(C(C)(C)O)CC3)ncc2n1] 2-{1-[4-({(1R)-1-[3-(difluoromethyl)-2-fluorophenyl]ethyl}amino)-2-methylpyrido[3,4- d]pyrimidin-6-yl]piperidin-4-yl}propan-2-ol ¹H-NMR (400 MHz, DMSO-d6) δ [ppm]: 1.071 (16.00), 1.107 (9.37), 1.271 (0.57), 1.298 (0.65), 1.424 (0.40), 1.454 (0.53), 1.608 (3.56), 1.625 (3.57), 1.819 (0.90), 1.850 (0.81), 2.293 (10.52), 2.327 (0.42), 2.518 (1.66), 2.523 (1.04), 2.669 (0.40), 2.706 (0.54), 2.738 (1.04), 2.769 (0.53), 4.167 (4.22), 4.191 (0.86), 4.470 (0.68), 4.499 (0.66), 5.751 (0.55), 5.769 (0.84), 5.787 (0.55), 7.102 (0.80), 7.238 (1.69), 7.277 (0.61), 7.296 (1.34), 7.315 (0.78), 7.374 (0.71), 7.415 (2.17), 7.485 (0.47), 7.502 (0.80), 7.636 (0.43), 7.654 (0.79), 8.415 (0.92), 8.433 (0.88), 8.638 (3.47). |
| 240 | 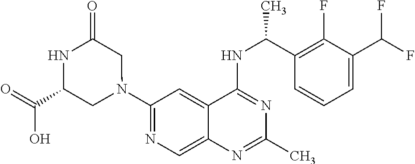 [SMILES: Cc1nc(N[C@H](C)c2cccc(C(F)F)c2F)c2cc(N3CC(=O)N[C@@H](C(=O)O)C3)ncc2n1] (2R)-4-[4-({(1R)-1-[3-(difluoromethyl)-2-fluorophenyl]ethyl}amino)-2-methylpyrido[3,4- d]pyrimidin-6-yl]-6-oxopiperazine-2-carboxylic acid ¹H-NMR (400 MHz, DMSO-d6) δ [ppm]: 0.833 (0.44), 0.850 (0.69), 1.013 (4.70), 1.031 (9.55), 1.049 (4.89), 1.131 (0.71), 1.148 (0.85), 1.165 (1.04), 1.231 (4.41), 1.613 (5.68), 1.631 (5.60), 2.303 (16.00), 2.322 (1.14), 2.326 (1.37), 2.331 (1.02), 2.518 (5.64), 2.522 (3.64), 2.539 (2.58), 2.664 (1.02), 2.668 (1.35), 2.673 (1.04), 2.694 (0.92), 2.712 (2.21), 2.730 (2.16), 2.748 (0.89), 2.870 (0.42), 3.869 (1.10), 3.913 (1.17), 3.955 (0.89), 4.038 (0.98), 4.082 (0.62), 4.094 (0.87), 4.137 (0.64), 4.210 (0.40), 5.751 (0.75), 5.769 (1.12), 5.786 (0.75), 7.105 (1.29), 7.241 (2.87), 7.277 (1.06), 7.296 (2.16), 7.315 (1.29), 7.377 (1.21), 7.398 (2.89), 7.480 (0.92), 7.497 (1.41), 7.515 (0.75), 7.634 (0.98), 7.653 (1.56), 7.671 (0.87), 8.223 (2.31), 8.582 (1.17), 8.599 (1.17), 8.675 (5.18). |
| 241 | 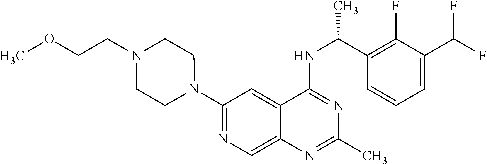 [SMILES: COCCN1CCN(c2cc3c(N[C@H](C)c4cccc(C(F)F)c4F)nc(C)nc3cn2)CC1] N-{(1R)-1-[3-(difluoromethyl)-2-fluorophenyl]ethyl]-6-[4-(2-methoxyethyl)piperazin-1- yl]-2-methylpyrido[3,4-d]pyrimidin-4-amine ¹H-NMR (400 MHz, DMSO-d6) δ [ppm]: 0.967 (0.73), 1.144 (0.44), 1.605 (3.45), 1.623 (3.46), 2.302 (9.84), 2.522 (2.84), 2.535 (2.20), 2.549 (3.29), 2.564 (2.09), 2.572 (2.14), 2.584 (2.78), 2.596 (2.10), 3.261 (16.00), 3.483 (1.67), 3.497 (3.48), 3.512 (1.64), 3.538 (1.89), 3.551 (2.49), 3.563 (1.87), 5.750 (0.54), 5.769 (0.85), 5.786 (0.54), 7.103 (0.78), 7.238 (1.64), 7.277 (0.60), 7.296 (1.32), 7.315 (0.78), 7.374 (0.70), 7.431 (2.13), 7.487 (0.47), 7.504 (0.80), 7.633 (0.45), 7.651 (0.79), 8.425 (0.88), 8.443 (0.85), 8.657 (3.12). |
| 242 | 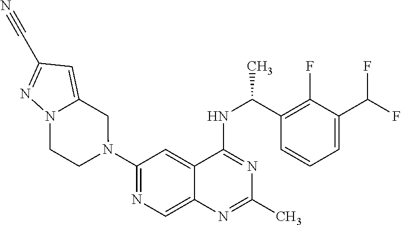 [SMILES: Cc1nc(N[C@H](C)c2cccc(C(F)F)c2F)c2cc(N3CCn4nc(C#N)cc4C3)ncc2n1] 5-[4-({(1R)-1-[3-(difluoromethyl)-2-fluorophenyl]ethyl}amino)-2-methylpyrido[3,4- d]pyrimidin-6-yl]-4,5,6,7-tetrahydropyrazolo[1,5-a]pyrazine-2-carbonitrile ¹H-NMR (400 MHz, DMSO-d6) δ [ppm]: 1.230 (1.38), 1.604 (1.24), 1.622 (1.55), 1.634 (4.84), 1.651 (4.74), 2.324 (16.00), 2.332 (1.85), 2.336 (0.84), 2.394 (3.36), 2.518 (6.32), 2.523 (4.44), 2.660 (0.47), 2.665 (1.08), 2.669 (1.55), 2.673 (1.11), 2.678 (0.47), 4.213 (0.94), 4.225 (1.88), 4.240 (1.48), 4.398 (1.45), 4.411 (2.02), 4.425 (0.97), 4.883 (4.91), 5.765 (0.84), 5.782 (1.18), 5.800 (0.74), 6.981 (4.91), 7.105 (1.21), 7.241 (2.55), 7.281 (0.97), 7.300 (2.08), 7.319 (1.21), 7.377 (1.04), 7.496 (0.74), 7.513 (1.24), 7.531 (0.61), 7.638 (2.92), 7.667 (1.14), 7.686 (0.74), 8.153 (0.50), 8.487 (1.18), 8.505 (1.11), 8.739 (5.18). |
| 243 | 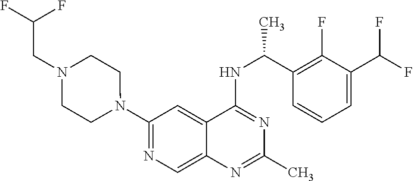 [SMILES: Cc1nc(N[C@H](C)c2cccc(C(F)F)c2F)c2cc(N3CCN(CC(F)F)CC3)ncc2n1] 6-[4-(2,2-difluoroethyl)piperazin-1-yl]-N-{(1R)-1-[3-(difluoromethyl)-2- fluorophenyl]ethyl}-2-methylpyrido[3,4-d]pyrimidin-4-amine ¹H-NMR (400 MHz, DMSO-d6) δ [ppm]: 0.967 (1.15), 1.107 (2.19), 1.143 (0.69), 1.607 (5.72), 1.624 (5.76), 2.304 (16.00), 2.322 (1.05), 2.326 (1.25), 2.331 (0.94), 2.522 (5.05), 2.539 (1.87), 2.664 (0.86), 2.668 (1.15), 2.673 (0.94), 2.692 (3.11), 2.703 (4.42), 2.715 (3.37), 2.776 (1.09), 2.787 (1.12), 2.815 (2.19), 2.826 (2.22), 2.855 (1.10), 2.865 (1.07), 3.559 (3.26), 3.571 (4.24), 3.583 (3.16), 5.751 (0.89), 5.768 (1.43), 5.786 (0.92), 6.087 (0.69), 6.216 (0.66), 6.226 (1.45), 6.238 (0.67), 6.366 (0.66), 7.103 (1.28), 7.238 (2.65), 7.277 (1.02), 7.296 (2.20), 7.315 (1.28), 7.374 (1.13), 7.448 (3.57), 7.488 (0.81), 7.505 (1.33), 7.523 (0.66), 7.632 (0.76), 7.650 (1.30), 7.668 (0.64), 8.425 (1.41), 8.444 (1.40), 8.663 (5.25). |
| 244 | 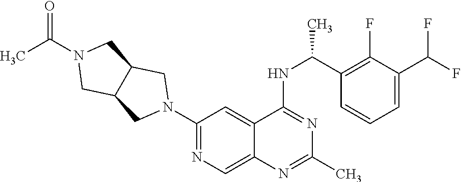 [SMILES: CC(=O)N1C[C@@H]2CN(c3cc4c(N[C@H](C)c5cccc(C(F)F)c5F)nc(C)nc4cn3)C[C@@H]2C1] 1-[5-[4-({(1R)-1-[3-(difluoromethyl)-2-fluorophenyl]ethyl}amino)-2-methylpyrido[3,4- d]pyrimidin-6-yl]hexahydropyrrolo[3,4-c]pyrrol-2(1H)-yl]ethan-1-one (mixture of stereoisomers) ¹H-NMR (400 MHz, DMSO-d6) δ [ppm]: 1.230 (0.45), 1.603 (5.47), 1.621 (5.54), 1.751 (0.71), 1.929 (1.18), 1.949 (16.00), 2.292 (13.15), 2.323 (0.99), 2.327 (0.70), 2.331 (0.47), 2.518 (2.56), 2.523 (1.63), 2.665 (0.44), 2.669 (0.59), 2.673 (0.45), 2.914 (0.77), 3.038 (0.64), 3.051 (0.74), 3.120 (0.71), 3.137 (0.66), 3.268 (0.59), 3.279 (0.61), 3.290 (0.68), 3.299 (1.16), 3.310 (0.96), 3.354 (1.27), 3.369 (1.42), 3.376 (1.30), 3.381 (1.31), 3.388 (1.13), 3.402 (1.06), 3.414 (0.64), 3.431 (0.79), 3.440 (0.82), 3.457 (1.02), 3.467 (0.95), 3.571 (0.64), 3.577 (0.65), 3.589 (0.68), 3.597 (0.79), 3.608 (0.62), 3.620 (0.57), 3.627 (0.54), 3.689 (0.75), 3.704 (1.66), 3.722 (1.28), 3.731 (1.47), 3.750 (1.76), 3.770 (1.20), 3.777 (1.15), 3.796 (0.88), 5.763 (0.66), 5.781 (1.00), 5.798 (0.67), 7.094 (3.67), 7.101 (1.64), 7.236 (2.62), 7.272 (0.99), 7.291 (2.12), 7.310 (1.23), 7.372 (1.11), 7.484 (0.80), 7.501 (1.35), 7.519 (0.71), 7.636 (0.69), 7.653 (1.24), 7.672 (0.67), 8.375 (1.41), 8.394 (1.36), 8.634 (5.05). |
| 245 | 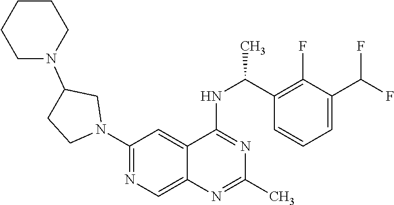 [SMILES: Cc1nc(N[C@H](C)c2cccc(C(F)F)c2F)c2cc(N3CCC(N4CCCCC4)C3)ncc2n1] N-{(1R)-1-[3-(difluoromethyl)-2-fluorophenyl]ethyl}-2-methyl-6-[3-(piperidin-1- yl)pyrrolidin-1-yl]pyrido[3,4-d]pyrimidin-4-amine (mixture of stereoisomers) ¹H-NMR (400 MHz, DMSO-d6) δ [ppm]: 1.107 (3.65), 1.413 (1.56), 1.425 (1.50), 1.525 (3.09), 1.538 (3.70), 1.551 (2.46), 1.609 (5.87), 1.626 (5.89), 1.827 (0.55), 1.852 (0.82), 1.878 (0.60), 2.212 (0.74), 2.228 (0.75), 2.240 (0.66), 2.257 (0.42), 2.285 (16.00), 2.336 (0.41), 2.441 (1.44), 2.518 (4.88), 2.523 (3.24), 2.933 (0.61), 2.953 (0.80), 2.974 (0.61), 3.170 (0.59), 3.185 (0.71), 3.194 (0.83), 3.209 (0.87), 3.215 (0.67), 3.230 (0.53), 3.377 (0.59), 3.386 (0.63), 3.394 (0.47), 3.403 (1.08), 3.411 (0.48), 3.420 (0.61), 3.429 (0.60), 3.663 (0.91), 3.684 (0.81), 3.720 (0.52), 3.741 (0.94), 3.762 (0.86), 3.784 (0.41), 5.755 (0.61), 5.773 (1.02), 5.786 (1.01), 5.804 (0.59), 7.039 (4.35), 7.102 (1.54), 7.237 (3.26), 7.272 (1.19), 7.291 (2.61), 7.310 (1.50), 7.373 (1.35), 7.482 (0.89), 7.499 (1.51), 7.516 (0.74), 7.632 (0.84), 7.650 (1.51), 7.668 (0.74), 8.311 (1.56), 8.329 (1.49), 8.624 (6.23). |
| 246 | 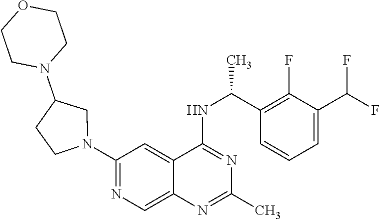 [SMILES: Cc1nc(N[C@H](C)c2cccc(C(F)F)c2F)c2cc(N3CCC(N4CCOCC4)C3)ncc2n1] N-{(1R)-1-[3-(difluoromethyl)-2-fluorophenyl]ethyl}-2-methyl-6-[3-(morpholin-4- yl)pyrrolidin-1-yl]pyrido[3,4-d]pyrimidin-4-amine (mixture of stereoisomers) ¹H-NMR (400 MHz, DMSO-d6) δ [ppm]: 1.608 (5.11), 1.625 (5.13), 1.888 (0.57), 1.913 (0.42), 2.228 (0.54), 2.242 (0.56), 2.254 (0.49), 2.288 (16.00), 2.332 (0.62), 2.518 (3.86), 2.522 (3.06), 2.539 (2.71), 2.673 (0.56), 2.989 (0.48), 3.237 (0.56), 3.248 (0.58), 3.396 (0.48), 3.403 (0.49), 3.420 (0.87), 3.437 (0.51), 3.445 (0.53), 3.611 (2.78), 3.622 (4.92), 3.633 (2.99), 3.656 (0.91), 3.677 (0.70), 3.750 (0.40), 3.767 (0.80), 3.793 (0.76), 5.758 (0.49), 5.767 (0.56), 5.776 (0.80), 5.786 (0.76), 5.793 (0.55), 5.803 (0.47), 7.056 (3.34), 7.101 (1.35), 7.237 (2.88), 7.271 (0.98), 7.290 (2.18), 7.309 (1.25), 7.373 (1.17), 7.483 (0.72), 7.499 (1.25), 7.518 (0.61), 7.634 (0.68), 7.652 (1.22), 7.670 (0.60), 8.332 (0.84), 8.350 (0.80), 8.629 (5.32). |
| 247 | 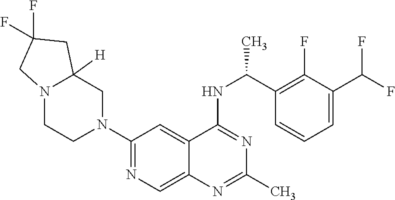 [SMILES: Cc1nc(N[C@H](C)c2cccc(C(F)F)c2F)c2cc(N3CCN4CC(F)(F)CC4C3)ncc2n1] 6-[7,7-difluorohexahydropyrrolo[1,2-a]pyrazin-2(1H)-yl]-N-{(1R)-1-[3-(difluoromethyl)-2- fluorophenyl]ethyl}-2-methylpyrido[3,4-d]pyrimidin-4-amine (mixture of stereoisomers) ¹H-NMR (400 MHz, DMSO-d6) δ [ppm]: 1.595 (0.61), 1.610 (7.14), 1.628 (6.92), 2.036 (0.46), 2.092 (0.44), 2.306 (16.00), 2.322 (1.22), 2.327 (1.57), 2.332 (1.31), 2.336 (0.78), 2.358 (0.94), 2.385 (0.57), 2.401 (1.38), 2.420 (0.50), 2.434 (0.42), 2.453 (0.79), 2.466 (1.18), 2.518 (3.89), 2.523 (2.84), 2.539 (1.62), 2.565 (0.52), 2.581 (0.70), 2.611 (0.68), 2.627 (0.46), 2.653 (1.05), 2.660 (1.16), 2.664 (1.03), 2.669 (1.29), 2.673 (1.07), 2.678 (1.05), 2.683 (1.09), 2.689 (0.81), 2.708 (0.55), 2.714 (0.54), 2.725 (0.89), 2.945 (0.57), 2.951 (0.46), 2.974 (0.98), 3.004 (0.50), 3.098 (1.03), 3.125 (1.00), 3.442 (0.52), 3.447 (0.54), 3.476 (1.00), 3.504 (0.52), 4.307 (0.74), 4.338 (0.72), 4.485 (1.05), 4.512 (1.01), 5.757 (0.94), 5.775 (1.46), 5.793 (0.94), 7.102 (1.64), 7.238 (3.49), 7.278 (0.96), 7.297 (2.07), 7.316 (1.20), 7.374 (1.44), 7.461 (3.95), 7.488 (0.94), 7.506 (1.53), 7.523 (0.76), 7.635 (0.76), 7.654 (1.38), 7.672 (0.72), 8.442 (1.73), 8.461 (1.64), 8.668 (6.64). |
| 248 | 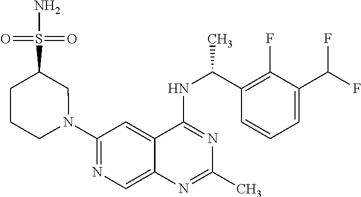 [SMILES: Cc1nc(N[C@H](C)c2cccc(C(F)F)c2F)c2cc(N3CCC[C@@H](S(N)(=O)=O)C3)ncc2n1] (3RS)-1-[4-({(1R)-1-[3-(difluoromethyl)-2-fluorophenyl]ethyl}amino)-2-methylpyrido[3,4- d]pyrimidin-6-yl]piperidine-3-sulfonamide ¹H-NMR (400 MHz, DMSO-d6) δ [ppm]: 1.104 (3.51), 1.137 (1.33), 1.233 (0.39), 1.352 (0.47), 1.612 (7.34), 1.629 (7.49), 1.663 (1.09), 1.686 (0.94), 1.723 (0.70), 1.907 (1.09), 1.941 (0.86), 2.075 (1.17), 2.085 (1.40), 2.116 (0.55), 2.222 (0.86), 2.251 (0.62), 2.302 (12.72), 2.307 (12.96), 2.318 (1.80), 2.323 (3.51), 2.327 (4.84), 2.332 (3.36), 2.337 (1.48), 2.518 (16.00), 2.523 (10.85), 2.540 (1.72), 2.660 (1.40), 2.665 (3.36), 2.669 (4.68), 2.674 (3.20), 2.679 (1.40), 2.831 (0.78), 2.859 (2.03), 2.890 (1.64), 2.983 (0.62), 3.006 (0.86), 3.074 (1.33), 4.161 (0.47), 4.194 (0.86), 4.221 (0.47), 5.055 (0.70), 5.747 (0.62), 5.756 (0.70), 5.765 (1.01), 5.774 (1.01), 5.792 (0.62), 6.939 (7.49), 7.103 (1.64), 7.240 (3.43), 7.278 (0.86), 7.297 (1.95), 7.317 (1.17), 7.375 (1.48), 7.501 (5.23), 7.523 (0.86), 7.634 (0.78), 7.651 (1.33), 7.670 (0.70), 8.162 (3.75), 8.470 (1.09), 8.478 (1.17), 8.488 (1.17), 8.496 (1.01), 8.679 (7.02). |
| 249 | 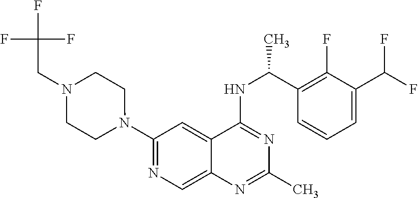 [SMILES: Cc1nc(N[C@H](C)c2cccc(C(F)F)c2F)c2cc(N3CCN(CC(F)(F)F)CC3)ncc2n1] N-{(1R)-1-[3-(difluoromethyl)-2-fluorophenyl]ethyl]-2-methyl-6-[4-(2,2,2- trifluoroethyl)piperazin-1-yl]pyrido[3,4-d]pyrimidin-4-amine ¹H-NMR (400 MHz, DMSO-d6) δ [ppm]: 0.860 (0.87), 0.967 (2.86), 1.107 (1.03), 1.109 (0.63), 1.144 (1.71), 1.224 (0.63), 1.388 (0.42), 1.607 (5.30), 1.625 (5.28), 2.305 (16.00), 2.318 (0.70), 2.323 (1.13), 2.327 (1.55), 2.332 (1.10), 2.336 (0.49), 2.518 (4.95), 2.523 (3.33), 2.660 (0.45), 2.665 (1.06), 2.669 (1.50), 2.673 (1.06), 2.678 (0.47), 2.779 (2.56), 2.791 (3.59), 2.803 (2.74), 3.237 (0.84), 3.262 (2.49), 3.287 (2.35), 3.313 (1.08), 3.572 (2.77), 3.585 (3.54), 3.596 (2.65), 5.752 (0.80), 5.770 (1.24), 5.787 (0.80), 7.103 (1.22), 7.238 (2.60), 7.278 (0.89), 7.297 (1.97), 7.316 (1.13), 7.374 (1.08), 7.454 (3.17), 7.490 (0.68), 7.506 (1.15), 7.524 (0.56), 7.634 (0.61), 7.651 (1.13), 7.670 (0.56), 8.423 (1.29), 8.441 (1.27), 8.666 (4.86). |
| 250 | 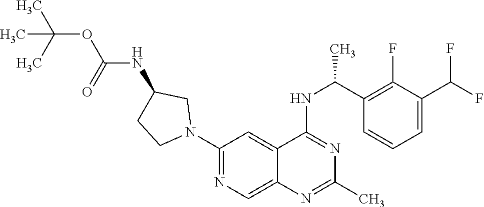 [SMILES: Cc1nc(N[C@H](C)c2cccc(C(F)F)c2F)c2cc(N3CC[C@@H](NC(=O)OC(C)(C)C)C3)ncc2n1] tert-butyl {(3R)-1-[4-({(1R)-1-[3-(difluoromethyl)-2-fluorophenyl]ethyl}amino)-2- methylpyrido[3,4-d]pyrimidin-6-yl]pyrrolidin-3-yl}carbamate ¹H-NMR (400 MHz, DMSO-d6) δ [ppm]: 1.154 (1.72), 1.172 (3.73), 1.190 (1.92), 1.402 (16.00), 1.605 (3.35), 1.622 (3.37), 1.952 (0.40), 1.987 (7.22), 2.286 (10.59), 2.518 (1.02), 2.523 (0.66), 3.267 (0.40), 3.279 (0.44), 3.293 (0.49), 3.306 (0.53), 3.626 (0.44), 3.662 (0.50), 3.678 (0.54), 3.688 (0.46), 3.999 (0.52), 4.017 (1.53), 4.034 (1.47), 4.053 (0.48), 4.176 (0.41), 5.758 (0.62), 5.776 (0.81), 5.794 (0.52), 7.062 (2.14), 7.100 (0.79), 7.237 (1.72), 7.271 (1.15), 7.290 (1.81), 7.310 (0.78), 7.373 (0.69), 7.481 (0.46), 7.498 (0.78), 7.625 (0.42), 7.642 (0.74), 8.398 (0.78), 8.416 (0.73), 8.621 (3.01). |
| 251 | 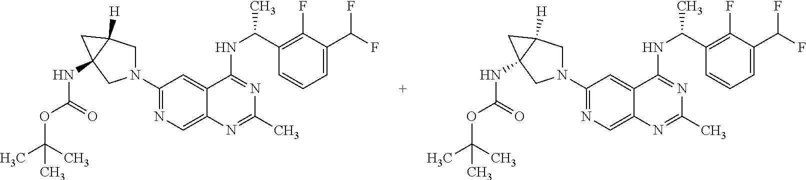 [SMILES: Cc1nc(N[C@H](C)c2cccc(C(F)F)c2F)c2cc(N3C[C@H]4C[C@@]4(NC(=O)OC(C)(C)C)C3)ncc2n1 ; Cc1nc(N[C@H](C)c2cccc(C(F)F)c2F)c2cc(N3C[C@@H]4C[C@]4(NC(=O)OC(C)(C)C)C3)ncc2n1] tert-butyl {3-[4-({(1R)-1-[3-(difluoromethyl)-2-fluorophenyl]ethyl}amino)-2- methylpyrido[3,4-d]pyrimidin-6-yl]-3-azabicyclo[3.1.0]hexan-1-yl}carbamate (mixture of stereoisomers) ¹H-NMR (400 MHz, DMSO-d6) δ [ppm]: 0.734 (0.79), 0.746 (0.45), 0.930 (0.71), 0.946 (0.72), 1.401 (16.00), 1.602 (2.32), 1.620 (2.33), 1.752 (0.59), 2.285 (4.13), 2.287 (4.17), 2.518 (1.03), 2.523 (0.71), 3.389 (0.51), 7.087 (1.20), 7.101 (0.58), 7.238 (1.10), 7.288 (0.54), 7.293 (0.56), 7.374 (0.47), 7.498 (0.54), 7.608 (0.50), 7.647 (0.41), 8.610 (1.93). |
| 252 | 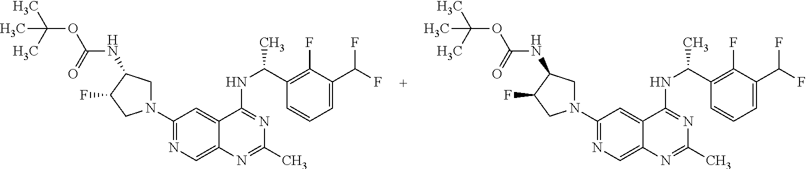 [SMILES: Cc1nc(N[C@H](C)c2cccc(C(F)F)c2F)c2cc(N3C[C@H](F)[C@H](NC(=O)OC(C)(C)C)C3)ncc2n1 ; Cc1nc(N[C@H](C)c2cccc(C(F)F)c2F)c2cc(N3C[C@@H](F)[C@@H](NC(=O)OC(C)(C)C)C3)ncc2n1] tert-butyl {1-[4-({(1R)-1-[3-(difluoromethyl)-2-fluorophenyl]ethyl}amino)-2- methylpyrido[3,4-d]pyrimidin-6-yl]-4-fluoropyrrolidin-3-yl}carbamate (mixture of stereoisomers) ¹H-NMR (400 MHz, DMSO-d6) δ [ppm]: 1.231 (0.46), 1.433 (16.00), 1.608 (2.37), 1.626 (2.37), 2.294 (4.13), 2.298 (4.49), 2.326 (0.43), 2.518 (1.77), 2.522 (1.09), 2.669 (0.42), 3.314 (0.59), 3.828 (0.65), 3.847 (0.68), 3.870 (0.50), 5.774 (0.60), 7.102 (0.61), 7.121 (1.22), 7.238 (1.26), 7.291 (0.68), 7.374 (0.67), 7.400 (0.42), 7.502 (0.51), 7.647 (0.56), 8.402 (0.48), 8.420 (0.48), 8.648 (2.34). |
| 253 | 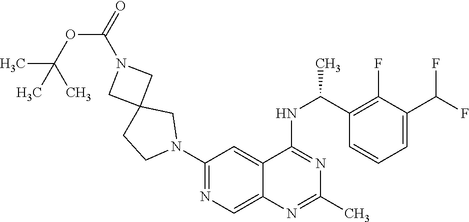 [SMILES: Cc1nc(N[C@H](C)c2cccc(C(F)F)c2F)c2cc(N3CCC4(CN(C(=O)OC(C)(C)C)C4)C3)ncc2n1] tert-butyl 6-[4-({(1R)-1-[3-(difluoromethyl)-2-fluorophenyl]ethyl}amino)-2- methylpyrido[3,4-d]pyrimidin-6-yl]-2,6-diazaspiro[3.4]octane-2-carboxylate ¹H-NMR (400 MHz, DMSO-d6) δ [ppm]: 1.392 (16.00), 1.605 (1.89), 1.623 (1.89), 2.197 (0.50), 2.213 (1.04), 2.230 (0.55), 2.289 (5.27), 2.522 (0.87), 3.519 (0.59), 3.526 (0.59), 3.655 (1.94), 3.867 (0.90), 5.780 (0.47), 7.079 (1.22), 7.101 (0.45), 7.237 (0.90), 7.293 (0.73), 7.312 (0.43), 7.502 (0.45), 7.652 (0.45), 8.364 (0.51), 8.382 (0.49), 8.625 (1.73). |
| 254 | 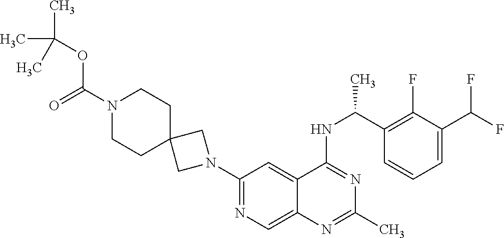 [SMILES: Cc1nc(N[C@H](C)c2cccc(C(F)F)c2F)c2cc(N3CC4(CCN(C(=O)OC(C)(C)C)CC4)C3)ncc2n1] tert-butyl 2-[4-({(1R)-1-[3-(difluoromethyl)-2-fluorophenyl]ethyl}amino)-2- methylpyrido[3,4-d]pyrimidin-6-yl]-2,7-diazaspiro[3.5]nonane-7-carboxylate ¹H-NMR (400 MHz, DMSO-d6) δ [ppm]: 1.387 (0.57), 1.406 (16.00), 1.593 (1.89), 1.611 (1.89), 1.720 (0.90), 1.735 (1.23), 1.748 (0.96), 2.296 (5.22), 2.518 (0.81), 2.523 (0.51), 3.776 (4.18), 5.765 (0.44), 7.100 (0.46), 7.113 (1.31), 7.236 (0.92), 7.293 (0.74), 7.312 (0.43), 7.503 (0.44), 7.652 (0.43), 8.392 (0.45), 8.411 (0.44), 8.613 (1.70). |
| 255 | 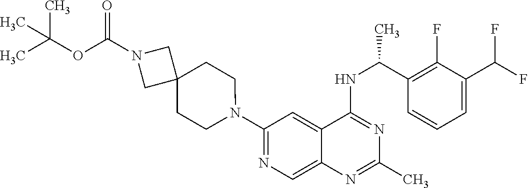 [SMILES: Cc1nc(N[C@H](C)c2cccc(C(F)F)c2F)c2cc(N3CCC4(CC3)CN(C(=O)OC(C)(C)C)C4)ncc2n1] tert-butyl 7-[4-({(1R)-1-[3-(difluoromethyl)-2-fluorophenyl]ethyl}amino)-2- methylpyrido[3,4-d]pyrimidin-6-yl]-2,7-diazaspiro[3.5]nonane-2-carboxylate ¹H-NMR (400 MHz, DMSO-d6) δ [ppm]: 1.365 (0.47), 1.374 (0.85), 1.391 (16.00), 1.606 (1.91), 1.624 (1.90), 1.767 (0.90), 1.781 (1.59), 1.793 (0.93), 2.294 (5.36), 2.523 (0.40), 3.571 (0.76), 3.634 (0.73), 5.766 (0.43), 7.102 (0.43), 7.238 (0.91), 7.296 (0.75), 7.315 (0.44), 7.451 (1.16), 7.504 (0.46), 7.646 (0.45), 8.428 (0.44), 8.446 (0.42), 8.641 (1.84). |
| 256 | 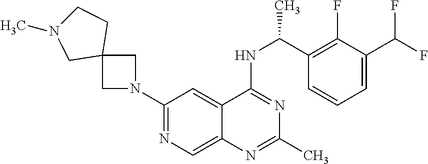 [SMILES: Cc1nc(N[C@H](C)c2cccc(C(F)F)c2F)c2cc(N3CC4(CCN(C)C4)C3)ncc2n1] N-{(1R)-1-[3-(difluoromethyl)-2-fluorophenyl]ethyl}-2-methyl-6-(6-methyl-2,6- diazaspiro[3.4]octan-2-yl)pyrido[3,4-d]pyrimidin-4-amine ¹H-NMR (400 MHz, DMSO-d6) δ [ppm]: 1.107 (0.63), 1.224 (0.66), 1.230 (0.47), 1.597 (5.75), 1.615 (5.73), 2.134 (1.06), 2.151 (1.90), 2.169 (1.17), 2.264 (0.42), 2.297 (16.00), 2.318 (1.22), 2.322 (1.48), 2.327 (1.64), 2.332 (1.29), 2.377 (2.79), 2.454 (0.52), 2.518 (4.55), 2.523 (2.86), 2.539 (0.61), 2.627 (0.68), 2.665 (1.48), 2.669 (1.76), 2.673 (1.41), 2.885 (0.78), 3.277 (0.68), 3.295 (2.63), 3.920 (1.43), 3.931 (1.45), 3.940 (2.39), 3.951 (2.51), 3.979 (2.53), 3.989 (2.42), 3.998 (1.38), 4.008 (1.41), 5.746 (0.85), 5.763 (1.32), 5.781 (0.85), 7.099 (1.31), 7.131 (3.57), 7.235 (2.74), 7.271 (0.98), 7.290 (2.09), 7.309 (1.19), 7.371 (1.13), 7.485 (0.78), 7.502 (1.27), 7.520 (0.61), 7.637 (0.70), 7.656 (1.24), 7.673 (0.61), 8.418 (1.32), 8.436 (1.24), 8.621 (4.78). |
| 257 | 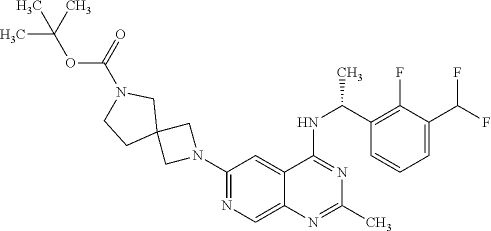 [SMILES: Cc1nc(N[C@H](C)c2cccc(C(F)F)c2F)c2cc(N3CC4(CCN(C(=O)OC(C)(C)C)C4)C3)ncc2n1] tert-butyl 2-[4-({(1R)-1-[3-(difluoromethyl)-2-fluorophenyl]ethyl}amino)-2- methylpyrido[3,4-d]pyrimidin-6-yl]-2,6-diazaspiro[3.4]octane-6-carboxylate ¹H-NMR (400 MHz, DMSO-d6) δ [ppm]: 1.381 (0.49), 1.408 (16.00), 1.593 (2.30), 1.611 (2.33), 2.109 (0.56), 2.123 (0.50), 2.136 (0.50), 2.301 (6.26), 3.317 (0.93), 3.477 (0.81), 3.502 (0.95), 3.915 (0.59), 3.922 (0.49), 3.935 (1.34), 3.942 (1.45), 3.955 (1.32), 3.975 (0.47), 5.764 (0.56), 7.100 (0.51), 7.136 (1.59), 7.236 (1.08), 7.273 (0.40), 7.293 (0.90), 7.312 (0.52), 7.371 (0.46), 7.504 (0.56), 7.648 (0.55), 8.385 (0.46), 8.403 (0.45), 8.627 (2.05). |

### EXAMPLE 258

### methyl 4-(2-{4-[4-({(1R)-1-[3-(difluoromethyl)-2-fluorophenyl]ethyl}amino)-2-methylpyrido[3,4-d]pyrimidin-6-yl]piperazin-1-yl}ethoxy)benzoate

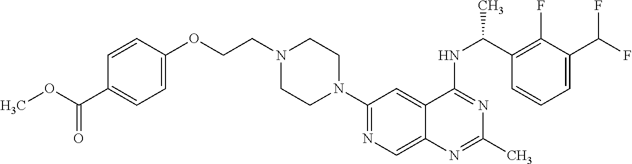 [SMILES: COC(=O)c1ccc(OCCN2CCN(c3cc4c(N[C@H](C)c5cccc(C(F)F)c5F)nc(C)nc4cn3)CC2)cc1]

A mixture of Example 2 (50.0 mg, 143 μmol), methyl 4-[2-(piperazin-1-yl)ethoxy]benzoate hydrochloride (144 mg, 428 μmol) and DIPEA (150 μl, 860 μmol) in DMSO (1 ml) was heated at 130° C. for 16 h. The titled compound was isolated (10 mg, 20%) after preparative HPLC purification (basic method).

¹H-NMR (400 MHz, DMSO-d6) δ [ppm]: 1.594 (1.73), 1.605 (4.49), 1.612 (2.38), 1.623 (4.38), 2.302 (12.90), 2.318 (0.66), 2.322 (1.18), 2.327 (1.56), 2.332 (1.15), 2.336 (0.55), 2.401 (4.33), 2.518 (6.52), 2.523 (4.11), 2.539 (0.60), 2.665 (3.26), 2.669 (3.62), 2.674 (3.84), 2.688 (2.33), 2.725 (3.21), 2.800 (1.18), 2.813 (2.41), 2.827 (1.21), 3.582 (2.79), 3.813 (16.00), 4.231 (1.32), 4.245 (2.68), 4.259 (1.26), 5.750 (1.01), 5.768 (1.23), 5.786 (0.71), 7.069 (0.55), 7.076 (4.16), 7.081 (1.29), 7.094 (1.40), 7.099 (4.79), 7.237 (2.58), 7.275 (0.82), 7.295 (1.75), 7.314 (1.01), 7.373 (1.07), 7.445 (2.52), 7.488 (0.71), 7.504 (1.18), 7.523 (0.58), 7.632 (0.55), 7.650 (1.12), 7.667 (0.77), 7.898 (0.68), 7.905 (4.74), 7.911 (1.37), 7.923 (1.34), 7.928 (4.27), 7.935 (0.47), 7.945 (0.60), 8.426 (1.10), 8.445 (1.04), 8.659 (4.16).

### EXAMPLE 259

### 4-(2-{4-[4-({(1R)-1-[3-(difluoromethyl)-2-fluorophenyl]ethyl}amino)-2-methylpyrido[3,4-d]pyrimidin-6-yl]piperazin-1-yl}ethoxy)benzoic acid

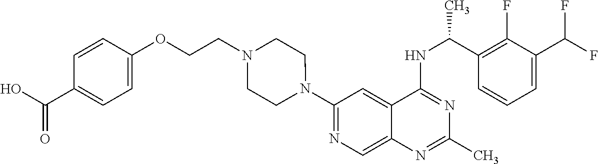 [SMILES: Cc1nc(N[C@H](C)c2cccc(C(F)F)c2F)c2cc(N3CCN(CCOc4ccc(C(=O)O)cc4)CC3)ncc2n1]

To a solution of Example 258 (8.20 mg, 13.8 μmol) in MeOH (2 ml) was added 1M NaOH (2 ml), additional MeOH (1 ml) needed for homogeneous solution. Stirred at RT for 16 h. Reaction was concentrated under reduced pressure to remove MeOH. Dissolved in DMSO:water (1:1) The titled compound was isolated (3.4 mg, 40%) after preparative HPLC purification (basic method).

¹H-NMR (400 MHz, DMSO-d6) δ [ppm]: 1.605 (5.44), 1.623 (5.45), 2.301 (16.00), 2.322 (0.47), 2.326 (0.52), 2.518 (1.82), 2.522 (1.17), 2.668 (4.18), 2.677 (3.07), 2.784 (1.43), 2.799 (2.90), 2.813 (1.53), 3.383 (2.18), 3.578 (4.14), 4.193 (1.62), 4.207 (3.21), 4.221 (1.57), 5.751 (0.87), 5.769 (1.34), 5.787 (0.85), 6.985 (3.99), 7.008 (4.09), 7.101 (1.25), 7.237 (2.74), 7.272 (0.99), 7.291 (2.13), 7.310 (1.23), 7.372 (1.10), 7.467 (3.18), 7.483 (0.87), 7.499 (1.31), 7.517 (0.64), 7.651 (0.70), 7.669 (1.28), 7.687 (0.64), 7.858 (5.09), 7.862 (1.65), 7.875 (1.64), 7.879 (4.58), 8.513 (1.21), 8.532 (1.14), 8.656 (5.55).

### EXAMPLE 260

### 6-(methanesulfonyl)-2-methyl-N-{(1R)-1-[3-(trifluoromethyl)phenyl]ethyl}pyrido[3,4-d]pyrimidin-4-amine

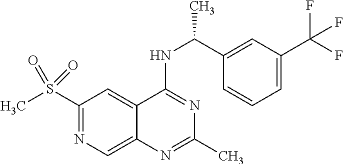 [SMILES: Cc1nc(N[C@H](C)c2cccc(C(F)(F)F)c2)c2cc(S(C)(=O)=O)ncc2n1]

A mixture of Example 10 (50.0 mg, 143 μmol) and sodium methanesulfinate (72.9 mg, 714 μmol) in DMSO (1 ml) was heated at 130° C. for 16h. The titled compound was isolated (14 mg, 23%) after preparative HPLC purification (basic method).

¹H-NMR (400 MHz, DMSO-d6) δ [ppm]: 1.625 (1.19), 1.643 (1.20), 2.482 (4.12), 2.518 (0.67), 2.523 (0.44), 3.313 (4.58), 3.331 (16.00), 5.758 (4.13), 7.607 (0.44), 7.846 (0.49), 9.089 (1.10), 9.105 (0.97).

### EXAMPLE 261

### 6-[(3R)-3-aminopyrrolidin-1-yl]-N-{(1R)-1-[3-(difluoromethyl)-2-fluorophenyl]ethyl}-2-methylpyrido[3,4-d]pyrimidin-4-amine hydrochloride salt

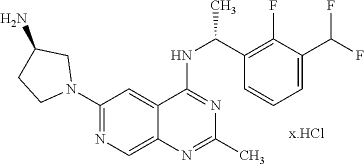 [SMILES: Cc1nc(N[C@H](C)c2cccc(C(F)F)c2F)c2cc(N3CC[C@@H](N)C3)ncc2n1]

To an ice-cooled solution of Example 250 (960 mg, 1.86 mmol) in dioxane (4.1 ml) was added HCL in dioxane (4.1 ml, 4.0 M, 16 mmol) and stirred for 3 h. The reaction was concentrated under reduced pressure to give the titled compound (904 mg) which was used without further purification.

¹H-NMR (400 MHz, DMSO-d6) δ [ppm]: 1.748 (2.93), 1.765 (2.93), 2.374 (0.53), 2.392 (0.44), 2.518 (0.95), 2.523 (0.71), 2.539 (11.48), 3.161 (8.17), 3.561 (0.42), 3.599 (0.49), 3.678 (0.48), 3.710 (0.67), 3.750 (0.58), 3.770 (0.48), 3.788 (0.74), 3.803 (0.82), 3.818 (0.46), 3.834 (0.46), 3.983 (0.54), 5.758 (16.00), 5.978 (0.63), 5.996 (0.94), 6.013 (0.59), 7.105 (1.06), 7.241 (2.23), 7.329 (0.50), 7.348 (1.07), 7.376 (1.06), 7.542 (0.51), 7.559 (0.84), 7.900 (0.72), 7.942 (0.49), 7.959 (0.61), 8.534 (0.83), 8.864 (2.51).

### EXAMPLE 262

### N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}cyclopropanecarboxamide

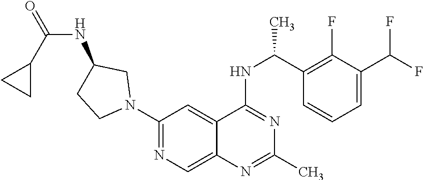 [SMILES: Cc1nc(N[C@H](C)c2cccc(C(F)F)c2F)c2cc(N3CC[C@@H](NC(=O)C4CC4)C3)ncc2n1]

To a solution of example 261 (50.0 mg, 110 μmol) and cyclopropanecarboxylic acid (18 μl, 220 μmol) in DMF (830 μl) was added DIPEA (96 μl, 550 μmol) and propylphosphonic anhydride solution (T3P) in DMF (130 μl, 50% purity, 220 μmol). Tot he reaction was added a few drops of water and the the titled compound (29 mg, 52%) was isolated after preparative HPLC (basc method).

¹H-NMR (400 MHz, DMSO-d6) δ [ppm]: 0.638 (1.36), 0.643 (1.84), 0.654 (1.40), 0.656 (1.63), 0.663 (1.86), 0.669 (1.89), 0.686 (2.52), 0.699 (1.20), 0.712 (0.46), 1.107 (0.61), 1.231 (0.74), 1.551 (0.64), 1.558 (0.74), 1.570 (1.12), 1.582 (0.67), 1.589 (0.76), 1.603 (5.13), 1.621 (5.06), 1.959 (0.59), 1.973 (0.64), 1.991 (0.46), 2.197 (0.48), 2.214 (0.61), 2.230 (0.54), 2.245 (0.41), 2.290 (16.00), 2.322 (0.56), 2.326 (0.72), 2.332 (0.51), 2.518 (3.04), 2.522 (1.87), 2.539 (0.41), 2.664 (0.49), 2.669 (0.69), 2.673 (0.51), 3.295 (0.72), 3.306 (0.89), 3.536 (0.49), 3.542 (0.58), 3.549 (0.48), 3.556 (0.62), 3.562 (0.56), 3.635 (0.41), 3.653 (0.90), 3.674 (1.17), 3.689 (1.07), 3.700 (1.00), 3.716 (0.76), 4.410 (0.39), 4.425 (0.66), 4.437 (0.66), 5.762 (0.77), 5.780 (1.18), 5.798 (0.77), 7.088 (3.16), 7.100 (1.28), 7.237 (2.53), 7.275 (0.87), 7.294 (1.92), 7.313 (1.12), 7.372 (1.02), 7.482 (0.66), 7.500 (1.09), 7.518 (0.54), 7.627 (0.59), 7.645 (1.09), 7.663 (0.54), 8.402 (1.35), 8.420 (2.61), 8.436 (1.41), 8.636 (4.72).

### EXAMPLE 263

### N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-2,2-difluoroacetamide

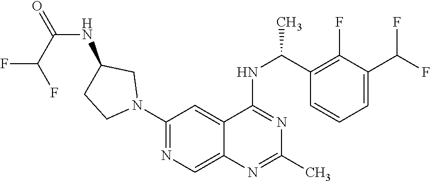 [SMILES: Cc1nc(N[C@H](C)c2cccc(C(F)F)c2F)c2cc(N3CC[C@@H](NC(=O)C(F)F)C3)ncc2n1]

Using the method described for Example 262: Example 261 (60 mg, 132 μmol) and difluoroacetic acid (17 μl, 260 μmol) gave the titled compound (39 mg, 56%) after preparative HPLC (basic method).

¹H-NMR (400 MHz, DMSO-d6) δ [ppm]: 1.107 (8.91), 1.605 (5.01), 1.622 (5.05), 2.056 (0.61), 2.069 (0.66), 2.088 (0.47), 2.253 (0.53), 2.270 (0.70), 2.293 (16.00), 2.322 (0.54), 2.327 (0.55), 2.518 (2.15), 2.523 (1.31), 2.539 (0.64), 2.669 (0.50), 3.401 (0.72), 3.411 (0.76), 3.428 (0.82), 3.438 (0.82), 3.543 (0.50), 3.549 (0.59), 3.562 (0.66), 3.568 (0.59), 3.582 (0.42), 3.628 (0.42), 3.646 (0.92), 3.663 (0.53), 3.672 (0.65), 3.725 (0.82), 3.741 (1.01), 3.752 (0.87), 3.768 (0.77), 4.190 (0.57), 4.507 (0.65), 4.520 (0.65), 5.763 (0.78), 5.781 (1.20), 5.800 (0.77), 6.079 (1.61), 6.214 (3.89), 6.348 (1.41), 7.100 (4.19), 7.237 (2.49), 7.276 (0.88), 7.295 (1.92), 7.314 (1.11), 7.372 (1.02), 7.484 (0.66), 7.501 (1.13), 7.520 (0.56), 7.629 (0.61), 7.646 (1.10), 7.664 (0.55), 8.398 (1.32), 8.416 (1.27), 8.644 (4.72), 9.160 (1.21), 9.178 (1.19).

### EXAMPLE 264

### N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-2-methoxyacetamide

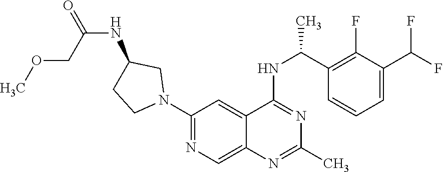 [SMILES: COCC(=O)N[C@@H]1CCN(c2cc3c(N[C@H](C)c4cccc(C(F)F)c4F)nc(C)nc3cn2)C1]

Using the method described for Example 262: Example 261 (50 mg, 110 μmol) and methoxyacetic acid (19.9 mg, 221 μmol) gave the titled compound (35 mg, 61%) after preparative HPLC (basic method).

¹H-NMR (400 MHz, DMSO-d6) δ [ppm]: 1.107 (0.99), 1.602 (2.93), 1.620 (2.94), 2.289 (9.20), 2.327 (0.42), 2.518 (1.78), 2.522 (1.10), 2.669 (0.42), 3.302 (16.00), 3.364 (0.58), 3.377 (0.52), 3.524 (0.43), 3.647 (0.41), 3.698 (0.49), 3.714 (0.58), 3.724 (0.52), 3.741 (0.45), 3.829 (4.30), 4.495 (0.43), 4.509 (0.42), 5.761 (0.45), 5.779 (0.69), 5.798 (0.45), 7.076 (1.85), 7.100 (0.69), 7.237 (1.45), 7.274 (0.50), 7.293 (1.13), 7.312 (0.65), 7.373 (0.60), 7.500 (0.64), 7.647 (0.64), 8.119 (0.80), 8.137 (0.79), 8.390 (0.76), 8.409 (0.73), 8.630 (2.77).

### EXAMPLE 265

### N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}oxetane-3-carboxamide

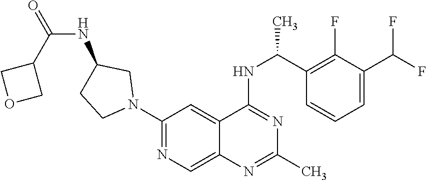 [SMILES: Cc1nc(N[C@H](C)c2cccc(C(F)F)c2F)c2cc(N3CC[C@@H](NC(=O)C4COC4)C3)ncc2n1]

Using the method described for Example 262: Example 261 (50 mg, 110 μmol) and oxetane-3-carboxylic acid (22.5 mg, 221 μmol) gave the titled compound (31 mg, 53%) after preparative HPLC (basic method).

¹H-NMR (400 MHz, DMSO-d6) δ [ppm]: 1.107 (1.39), 1.232 (0.90), 1.601 (5.07), 1.619 (5.07), 1.940 (0.62), 1.953 (0.64), 1.971 (0.45), 2.202 (0.51), 2.217 (0.62), 2.233 (0.56), 2.249 (0.41), 2.288 (16.00), 2.322 (0.66), 2.326 (0.90), 2.332 (0.64), 2.518 (3.21), 2.522 (1.90), 2.539 (0.43), 2.664 (0.60), 2.669 (0.83), 2.673 (0.62), 3.304 (0.88), 3.315 (1.16), 3.539 (0.60), 3.552 (0.66), 3.559 (0.60), 3.572 (0.49), 3.582 (0.47), 3.600 (1.01), 3.618 (0.53), 3.626 (0.58), 3.694 (0.79), 3.710 (1.03), 3.722 (1.50), 3.725 (1.26), 3.743 (1.71), 3.763 (1.01), 4.459 (0.64), 4.472 (0.64), 4.593 (5.37), 4.611 (7.94), 4.613 (4.98), 4.628 (3.32), 4.632 (3.19), 4.646 (0.41), 5.760 (0.77), 5.778 (1.22), 5.796 (0.77), 7.081 (3.14), 7.100 (1.22), 7.236 (2.57), 7.276 (0.90), 7.295 (1.95), 7.314 (1.11), 7.372 (1.05), 7.483 (0.66), 7.501 (1.11), 7.519 (0.53), 7.627 (0.60), 7.645 (1.09), 7.663 (0.53), 8.245 (1.35), 8.262 (1.33), 8.395 (1.30), 8.414 (1.24), 8.630 (4.77).

### EXAMPLE 266

### N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-1-methylazetidine-3-carboxamide

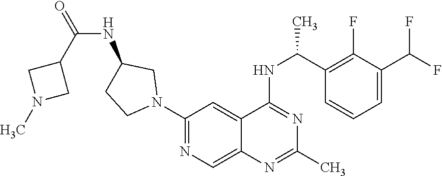 [SMILES: Cc1nc(N[C@H](C)c2cccc(C(F)F)c2F)c2cc(N3CC[C@@H](NC(=O)C4CN(C)C4)C3)ncc2n1]

Using the method described for Example 262: Example 261 (50 mg, 110 μmol) and 1-methylazetidine-3-carboxylic acid (25.4 mg, 221 μmol) gave the titled compound (12.5 mg, 21%) after preparative HPLC (basic method).

¹H-NMR (400 MHz, DMSO-d6) δ [ppm]: 1.103 (1.38), 1.107 (1.89), 1.224 (1.03), 1.230 (1.16), 1.601 (4.38), 1.619 (4.45), 1.826 (0.75), 1.926 (0.50), 1.940 (0.52), 2.160 (11.15), 2.183 (0.67), 2.198 (0.63), 2.214 (0.52), 2.288 (16.00), 2.322 (0.84), 2.327 (0.75), 2.332 (0.55), 2.518 (2.88), 2.523 (1.76), 2.539 (0.56), 2.665 (0.50), 2.669 (0.72), 2.673 (0.52), 3.038 (0.50), 3.055 (1.15), 3.072 (1.99), 3.077 (1.47), 3.087 (1.35), 3.104 (0.75), 3.122 (0.59), 3.268 (1.36), 3.278 (1.60), 3.294 (2.06), 3.346 (7.98), 3.460 (0.74), 3.496 (0.67), 3.510 (0.65), 3.516 (0.69), 3.523 (0.75), 3.531 (0.65), 3.537 (0.76), 3.542 (0.73), 3.556 (0.55), 3.592 (0.48), 3.610 (0.88), 3.618 (0.75), 3.628 (0.80), 3.636 (0.85), 3.644 (0.65), 3.679 (0.81), 3.694 (0.98), 3.706 (0.96), 3.721 (0.80), 4.415 (0.53), 4.428 (0.52), 5.759 (0.80), 5.779 (1.19), 5.796 (0.79), 7.064 (0.84), 7.074 (2.45), 7.100 (1.39), 7.236 (2.80), 7.275 (0.82), 7.295 (1.76), 7.314 (1.04), 7.372 (1.20), 7.483 (0.79), 7.500 (1.33), 7.518 (0.67), 7.628 (0.64), 7.646 (1.14), 7.665 (0.59), 8.161 (0.90), 8.178 (0.90), 8.398 (1.20), 8.416 (1.16), 8.629 (3.85).

### EXAMPLE 267

### methyl {(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}carbamate

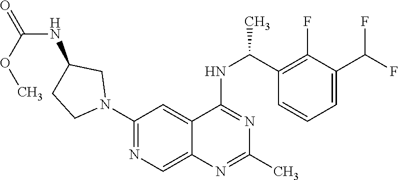 [SMILES: COC(=O)N[C@@H]1CCN(c2cc3c(N[C@H](C)c4cccc(C(F)F)c4F)nc(C)nc3cn2)C1]

To a mixture of Example 261 (50 mg, 110 μmol), triethylamine (77 μl, 550 μmol), DMAP (0.3 mg) in DCE (830 μl) was stirred at RT for 16 h. The reaction mixture was added water, extracted with DCM, washed with sat. NaCl. The organics were filtered through a hydrophobic filter and concentrated. The titled compound (13 mg, 24%) after preparative HPLC (basic method).

¹H-NMR (400 MHz, DMSO-d6) δ [ppm]: 1.107 (1.81), 1.231 (0.85), 1.603 (5.33), 1.621 (5.42), 1.952 (0.54), 1.966 (0.61), 1.987 (0.52), 2.187 (0.54), 2.205 (0.67), 2.219 (0.63), 2.237 (0.46), 2.288 (16.00), 2.322 (0.81), 2.327 (1.09), 2.331 (0.83), 2.518 (7.66), 2.523 (5.33), 2.539 (1.65), 2.665 (0.74), 2.669 (1.09), 2.673 (0.81), 3.512 (0.72), 3.552 (6.68), 3.606 (0.59), 3.624 (0.96), 3.642 (0.63), 3.648 (0.70), 3.675 (0.83), 3.691 (0.96), 3.702 (0.85), 3.718 (0.74), 4.203 (0.46), 4.219 (0.76), 4.231 (0.72), 4.245 (0.44), 5.759 (1.28), 5.778 (1.26), 5.796 (0.83), 7.068 (3.35), 7.101 (1.28), 7.237 (2.61), 7.274 (0.98), 7.293 (2.11), 7.312 (1.22), 7.373 (1.11), 7.483 (0.72), 7.500 (1.22), 7.517 (0.65), 7.570 (0.83), 7.586 (0.83), 7.628 (0.67), 7.646 (1.20), 7.664 (0.61), 8.393 (1.31), 8.411 (1.31), 8.625 (5.01).

### EXAMPLE 268

### N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}methanesulfonamide

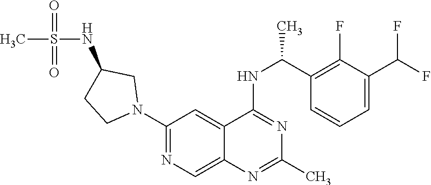 [SMILES: Cc1nc(N[C@H](C)c2cccc(C(F)F)c2F)c2cc(N3CC[C@@H](NS(C)(=O)=O)C3)ncc2n1]

Using the method described for Example 262: Example 261 (50 mg, 110 μmol) and methanesulfonyl chloride (17 μl, 220 μmol) gave the titled compound (27 mg, 46%) after preparative HPLC (basic method).

¹H-NMR (400 MHz, DMSO-d6) δ [ppm]: 1.107 (2.65), 1.231 (0.47), 1.609 (4.33), 1.626 (4.34), 1.999 (0.48), 2.012 (0.54), 2.030 (0.47), 2.290 (14.09), 2.309 (0.63), 2.322 (0.73), 2.326 (0.89), 2.332 (0.54), 2.518 (2.21), 2.522 (1.33), 2.669 (0.55), 3.004 (16.00), 3.318 (0.48), 3.363 (0.71), 3.377 (0.71), 3.389 (0.76), 3.403 (0.76), 3.459 (0.62), 3.467 (0.48), 3.477 (0.41), 3.485 (0.77), 3.625 (0.54), 3.632 (0.44), 3.639 (0.47), 3.646 (0.45), 3.761 (0.66), 3.777 (0.84), 3.787 (0.71), 3.803 (0.66), 4.105 (0.62), 4.120 (0.60), 5.763 (0.67), 5.781 (1.03), 5.799 (0.66), 7.084 (2.75), 7.101 (1.07), 7.237 (2.13), 7.274 (0.76), 7.293 (1.66), 7.312 (0.96), 7.373 (0.89), 7.483 (0.60), 7.499 (1.99), 7.514 (1.48), 7.630 (0.51), 7.648 (0.93), 7.667 (0.47), 8.407 (1.13), 8.426 (1.07), 8.636 (4.14).

### EXAMPLE 269

### N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}cyclopropanesulfonamide

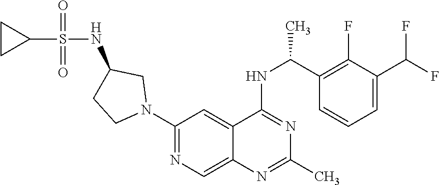 [SMILES: Cc1nc(N[C@H](C)c2cccc(C(F)F)c2F)c2cc(N3CC[C@@H](NS(=O)(=O)C4CC4)C3)ncc2n1]

Using the method described for Example 262: Example 261 (50 mg, 110 μmol) and cyclopropanesulfonyl chloride (22 μl, 220 μmol) gave the titled compound (31 mg, 51%) after preparative HPLC (basic method).

¹H-NMR (400 MHz, DMSO-d6) δ [ppm]: 0.946 (0.72), 0.952 (1.27), 0.960 (2.24), 0.963 (2.18), 0.972 (2.54), 0.977 (3.04), 0.987 (1.21), 0.990 (1.23), 0.993 (1.39), 0.997 (1.95), 1.016 (0.43), 1.107 (3.52), 1.231 (0.43), 1.608 (5.02), 1.626 (5.03), 2.010 (0.44), 2.028 (0.54), 2.041 (0.62), 2.059 (0.55), 2.290 (16.00), 2.322 (1.03), 2.327 (0.84), 2.332 (0.80), 2.518 (2.56), 2.523 (1.58), 2.539 (0.67), 2.635 (0.71), 2.639 (0.47), 2.651 (1.16), 2.659 (0.67), 2.665 (1.11), 2.669 (1.00), 3.384 (0.78), 3.397 (0.80), 3.410 (0.86), 3.423 (0.84), 3.464 (0.71), 3.471 (0.56), 3.482 (0.47), 3.488 (0.88), 3.634 (0.62), 3.639 (0.51), 3.647 (0.54), 3.653 (0.51), 3.772 (0.74), 3.789 (0.96), 3.799 (0.82), 3.816 (0.74), 4.115 (0.43), 4.131 (0.76), 4.147 (0.74), 5.763 (0.78), 5.780 (1.21), 5.798 (0.76), 7.085 (3.17), 7.101 (1.25), 7.237 (2.52), 7.273 (0.87), 7.293 (1.93), 7.312 (1.12), 7.373 (1.04), 7.483 (0.64), 7.501 (1.11), 7.518 (0.56), 7.539 (1.83), 7.556 (1.77), 7.630 (0.60), 7.648 (1.10), 7.665 (0.55), 8.401 (1.30), 8.420 (1.25), 8.637 (4.81).

TABLE 9
Examples 270-278
General method: To a solution of the carboxylic acid (230 μmol) in DMF (1 ml) was added HATU (230
μmol) and stirred for 15 mins, then DIPEA (766 μmol) and Example 148 (75 mg, 153 μmol) were
added. The reaction was stirred at RT. The compounds in Table 9 were then purified by preparative
HPLC (basic method) and/or silica chromatograpy.

| | Structure |
|---|---|
| | IUPAC-Name |
| Example | ¹H-NMR |
| 270 | 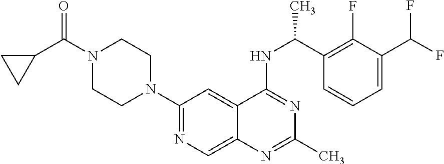 [SMILES: Cc1nc(N[C@H](C)c2cccc(C(F)F)c2F)c2cc(N3CCN(C(=O)C4CC4)CC3)ncc2n1] |
| | cyclopropyl{4-[4-({(1R)-1-[3-(difluoromethyl)-2-fluorophenyl]ethyl}amino)-2- |
| | methylpyrido[3,4-d]pyrimidin-6-yl]piperazin-1-yl}methanone |
| | ¹H-NMR (400 MHz, CHLOROFORM-d) δ [ppm]: 0.807 (0.58), 0.817 (1.80), 0.825 (2.01), |
| | 0.834 (1.18), 0.838 (1.83), 0.845 (2.12), 0.854 (0.75), 1.028 (0.72), 1.036 (2.17), 1.040 |
| | (2.39), 1.043 (2.07), 1.048 (2.33), 1.055 (2.02), 1.065 (0.63), 1.194 (0.98), 1.261 (0.86), |
| | 1.282 (3.14), 1.299 (0.88), 1.725 (4.80), 1.742 (4.75), 1.792 (0.64), 1.800 (0.70), 1.812 |
| | (1.19), 1.824 (0.64), 1.832 (0.57), 2.541 (16.00), 3.545 (0.84), 3.785 (0.86), 3.875 (1.52), |
| | 5.772 (0.67), 5.790 (1.10), 5.808 (0.81), 6.514 (2.01), 6.787 (1.05), 6.924 (2.12), 7.062 |
| | (1.00), 7.195 (0.81), 7.214 (1.80), 7.233 (1.03), 7.496 (0.58), 7.514 (1.00), 7.531 (1.03), |
| | 7.549 (0.99), 7.568 (0.49), 8.896 (3.92). |
| 271 | 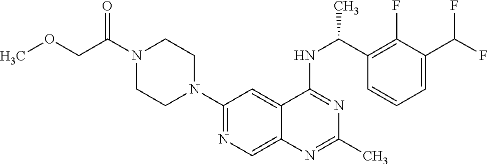 [SMILES: COCC(=O)N1CCN(c2cc3c(N[C@H](C)c4cccc(C(F)F)c4F)nc(C)nc3cn2)CC1] |
| | 1-{4-[4-({(1R)-1-[3-(difluoromethyl)-2-fluorophenyl]ethyl}amino)-2-methylpyrido[3,4- |
| | d]pyrimidin-6-yl]piperazin-1-yl}-2-methoxyethan-1-one |
| | ¹H-NMR (400 MHz, CHLOROFORM-d) δ [ppm]: 1.040 (0.98), 1.194 (0.63), 1.261 (0.57), |
| | 1.282 (0.93), 1.299 (0.63), 1.727 (3.38), 1.745 (3.32), 2.542 (11.32), 3.463 (16.00), 3.566 |
| | (0.59), 3.578 (0.93), 3.592 (0.75), 3.705 (4.64), 3.819 (0.76), 3.831 (0.94), 3.844 (0.61), |
| | 4.183 (7.42), 5.773 (0.47), 5.791 (0.76), 5.809 (0.54), 6.542 (0.91), 6.786 (0.76), 6.923 |
| | (1.53), 7.060 (0.72), 7.195 (0.58), 7.214 (1.26), 7.234 (0.72), 7.497 (0.40), 7.514 (0.70), |
| | 7.533 (0.66), 7.554 (0.67), 8.893 (2.72). |
| 272 | 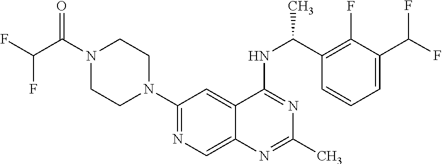 [SMILES: Cc1nc(N[C@H](C)c2cccc(C(F)F)c2F)c2cc(N3CCN(C(=O)C(F)F)CC3)ncc2n1] |
| | 1-{4-[4-({(1R)-1-[3-(difluoromethyl)-2-fluorophenyl]ethyl}amino)-2-methylpyrido[3,4- |
| | d]pyrimidin-6-yl]piperazin-1-yl}-2,2-difluoroethan-1-one |
| | ¹H-NMR (400 MHz, CHLOROFORM-d) δ [ppm]: 1.263 (0.87), 1.283 (1.44), 1.735 (4.97), |
| | 1.752 (4.95), 2.551 (16.00), 3.657 (1.28), 3.669 (1.95), 3.683 (1.96), 3.707 (1.31), 3.719 |
| | (1.94), 3.733 (1.89), 3.839 (2.67), 3.851 (3.40), 3.862 (2.13), 5.777 (0.69), 5.794 (1.12), |
| | 5.812 (0.79), 6.037 (1.37), 6.171 (2.71), 6.305 (1.25), 6.556 (1.01), 6.786 (1.15), 6.923 |
| | (2.32), 7.061 (1.08), 7.203 (0.88), 7.222 (1.96), 7.241 (1.11), 7.503 (0.64), 7.520 (1.10), |
| | 7.540 (0.86), 7.562 (1.01), 7.581 (0.51), 8.905 (4.06). |
| 273 | 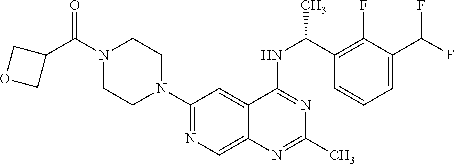 [SMILES: Cc1nc(N[C@H](C)c2cccc(C(F)F)c2F)c2cc(N3CCN(C(=O)C4COC4)CC3)ncc2n1] |
| | {4-[4-({(1R)-1-[3-(difluoromethyl)-2-fluorophenyl]ethyl}amino)-2-methylpyrido[3,4- |
| | d]pyrimidin-6-yl]piperazin-1-yl}(oxetan-3-yl)methanone |
| | ¹H-NMR (400 MHz, CHLOROFORM-d) δ [ppm]: 1.041 (1.04), 1.194 (1.12), 1.261 (0.95), |
| | 1.283 (3.70), 1.300 (0.68), 1.534 (0.41), 1.668 (1.04), 1.759 (5.41), 2.177 (0.61), 2.552 |
| | (16.00), 3.352 (1.34), 3.364 (1.79), 3.377 (1.61), 3.564 (1.33), 3.577 (1.92), 3.591 (1.64), |
| | 3.661 (1.71), 3.675 (1.91), 3.687 (1.38), 3.835 (1.59), 3.849 (1.84), 3.861 (1.28), 4.059 |
| | (0.70), 4.063 (0.68), 4.081 (1.33), 4.098 (0.76), 4.102 (0.74), 4.832 (2.57), 4.847 (3.58), |
| | 4.854 (2.68), 4.869 (3.04), 4.949 (3.25), 4.964 (3.28), 4.966 (3.65), 4.981 (2.46), 5.786 |
| | (0.70), 5.804 (1.10), 5.822 (0.75), 6.630 (0.43), 6.781 (1.20), 6.919 (2.39), 7.056 (1.16), |
| | 7.198 (0.96), 7.217 (2.10), 7.237 (1.23), 7.497 (0.71), 7.514 (1.20), 7.529 (0.80), 7.566 |
| | (0.55), 7.584 (0.96), 7.602 (0.50), 8.887 (4.11). |
| 274 | 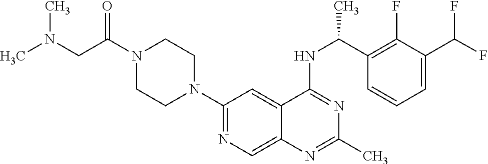 [SMILES: Cc1nc(N[C@H](C)c2cccc(C(F)F)c2F)c2cc(N3CCN(C(=O)CN(C)C)CC3)ncc2n1] |
| | 1-{4-[4-({(1R)-1-[3-(difluoromethyl)-2-fluorophenyl]ethyl}amino)-2-methylpyrido[3,4- |
| | d]pyrimidin-6-yl]piperazin-1-yl}-2-(dimethylamino)ethan-1-one |
| | ¹H-NMR (400 MHz, CHLOROFORM-d) δ [ppm]: 1.040 (0.55), 1.260 (1.31), 1.282 (0.73), |
| | 1.726 (3.04), 1.743 (2.99), 2.320 (16.00), 2.539 (9.87), 3.192 (4.45), 3.562 (0.64), 3.575 |
| | (1.00), 3.588 (0.84), 3.666 (0.65), 3.678 (1.00), 3.692 (0.89), 3.811 (2.22), 3.825 (2.32), |
| | 3.837 (1.70), 5.786 (0.68), 5.804 (0.59), 5.827 (0.66), 6.507 (1.84), 6.788 (0.67), 6.926 |
| | (1.35), 7.063 (0.64), 7.197 (0.52), 7.217 (1.15), 7.236 (0.66), 7.516 (0.67), 7.532 (0.67), |
| | 7.548 (0.70), 8.894 (2.45). |
| 275 | 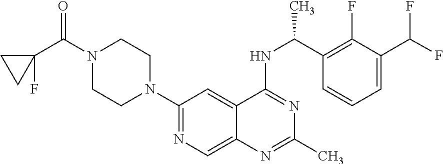 [SMILES: Cc1nc(N[C@H](C)c2cccc(C(F)F)c2F)c2cc(N3CCN(C(=O)C4(F)CC4)CC3)ncc2n1] |
| | {4-[4-({(1R)-1-[3-(difluoromethyl)-2-fluorophenyl]ethyl}amino)-2-methylpyrido[3,4- |
| | d]pyrimidin-6-yl]piperazin-1-yl}(1-fluorocyclopropyl)methanone |
| | ¹H-NMR (400 MHz, CHLOROFORM-d) δ [ppm]: 0.838 (1.24), 0.855 (1.67), 0.865 (1.59), |
| | 0.872 (1.51), 0.882 (2.41), 0.888 (1.69), 0.898 (1.14), 0.920 (1.23), 0.967 (0.56), 1.035 |
| | (4.01), 1.089 (0.46), 1.127 (0.52), 1.143 (0.53), 1.255 (16.00), 1.276 (9.79), 1.294 (3.52), |
| | 1.309 (1.71), 1.313 (1.85), 1.321 (2.03), 1.334 (1.49), 1.346 (2.71), 1.353 (1.37), 1.358 |
| | (1.34), 1.382 (0.52), 1.429 (0.43), 1.528 (0.76), 1.717 (4.05), 1.735 (4.25), 1.913 (0.55), |
| | 2.177 (1.46), 2.531 (10.50), 3.686 (0.85), 3.866 (0.40), 5.778 (0.46), 5.795 (0.74), 5.814 |
| | (0.53), 6.630 (0.89), 6.785 (0.71), 6.922 (1.40), 7.060 (0.68), 7.187 (0.56), 7.207 (1.19), |
| | 7.226 (0.69), 7.487 (0.43), 7.505 (0.72), 7.537 (0.41), 7.555 (0.67), 8.889 (2.43). |
| 276 | 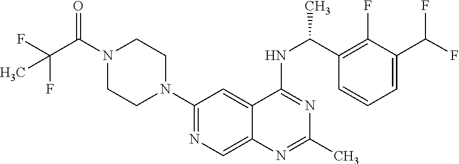 [SMILES: Cc1nc(N[C@H](C)c2cccc(C(F)F)c2F)c2cc(N3CCN(C(=O)C(C)(F)F)CC3)ncc2n1] |
| | 1-{4-[4-({(1R)-1-[3-(difluoromethyl)-2-fluorophenyl]ethyl}amino)-2-methylpyrido[3,4- |
| | d]pyrimidin-6-yl]piperazin-1-yl}-2,2-difluoropropan-1-one |
| | ¹H-NMR (400 MHz, CHLOROFORM-d) δ [ppm]: 1.040 (0.81), 1.261 (0.94), 1.282 (1.85), |
| | 1.299 (0.54), 1.725 (4.57), 1.742 (4.46), 1.841 (2.72), 1.891 (5.66), 1.941 (2.53), 2.534 |
| | (0.60), 2.543 (16.00), 3.648 (1.16), 3.659 (2.78), 3.673 (2.91), 3.684 (1.59), 3.834 (1.30), |
| | 3.848 (1.52), 3.860 (0.99), 3.927 (1.21), 3.941 (1.46), 3.953 (0.99), 5.769 (0.61), 5.787 |
| | (1.04), 5.804 (0.86), 5.822 (0.47), 5.831 (0.60), 6.512 (2.62), 6.786 (1.03), 6.923 (2.07), |
| | 7.061 (0.97), 7.196 (0.80), 7.216 (1.74), 7.235 (1.00), 7.499 (0.57), 7.516 (0.99), 7.529 |
| | (0.88), 7.546 (0.99), 7.564 (0.48), 8.898 (3.68). |
| 277 | 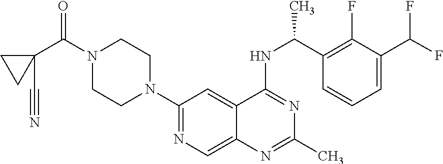 [SMILES: Cc1nc(N[C@H](C)c2cccc(C(F)F)c2F)c2cc(N3CCN(C(=O)C4(C#N)CC4)CC3)ncc2n1] |
| | 1-{4-[4-({(1R)-1-[3-(difluoromethyl)-2-fluorophenyl]ethyl}amino)-2-methylpyrido[3,4- |
| | d]pyrimidin-6-yl]piperazine-1-carbonyl}cyclopropane-1-carbonitrile |
| | ¹H-NMR (400 MHz, CHLOROFORM-d) δ [ppm]: 1.041 (0.96), 1.194 (1.24), 1.261 (0.43), |
| | 1.283 (2.83), 1.300 (0.70), 1.575 (1.30), 1.580 (1.23), 1.588 (2.30), 1.595 (3.27), 1.604 |
| | (2.44), 1.613 (0.71), 1.618 (1.05), 1.642 (1.14), 1.655 (2.65), 1.664 (3.75), 1.670 (2.09), |
| | 1.673 (1.92), 1.679 (1.40), 1.685 (1.62), 1.762 (5.62), 2.551 (16.00), 3.714 (1.03), 4.037 |
| | (0.53), 5.786 (0.76), 5.804 (1.17), 5.822 (0.80), 6.601 (0.84), 6.791 (1.20), 6.928 (2.45), |
| | 7.065 (1.15), 7.203 (0.94), 7.222 (2.07), 7.241 (1.21), 7.501 (0.69), 7.518 (1.20), 7.534 |
| | (0.64), 7.559 (0.59), 7.578 (1.05), 7.595 (0.53), 8.902 (4.20). |
| 278 | 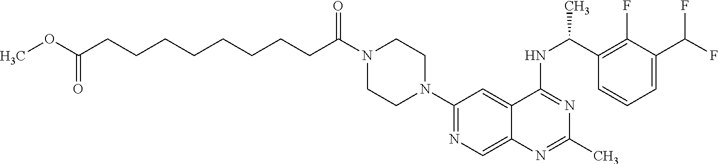 [SMILES: COC(=O)CCCCCCCCC(=O)N1CCN(c2cc3c(N[C@H](C)c4cccc(C(F)F)c4F)nc(C)nc3cn2)CC1] |
| | methyl 10-{4-[4-({(1R)-1-[3-(difluoromethyl)-2-fluorophenyl]ethyl}amino)-2- |
| | methylpyrido[3,4-d]pyrimidin-6-yl]piperazin-1-yl}-10-oxodecanoate |
| | ¹H-NMR (400 MHz, DMSO-d6) δ [ppm]: 1.267 (4.34), 1.511 (1.60), 1.612 (3.27), 1.629 |
| | (3.33), 2.265 (1.77), 2.283 (3.37), 2.291 (0.86), 2.301 (2.24), 2.309 (9.47), 2.322 (0.82), |
| | 2.327 (0.89), 2.331 (0.65), 2.351 (1.13), 2.370 (1.75), 2.388 (1.01), 2.523 (2.34), 2.665 |
| | (0.47), 2.669 (0.68), 2.673 (0.49), 3.129 (0.94), 3.302 (0.69), 3.534 (0.98), 3.568 (16.00), |
| | 3.620 (4.56), 5.756 (0.51), 5.774 (0.80), 5.791 (0.51), 7.103 (0.77), 7.239 (1.61), 7.277 |
| | (0.60), 7.297 (1.25), 7.317 (0.76), 7.374 (0.67), 7.480 (2.02), 7.508 (0.78), 7.636 (0.43), |
| | 7.653 (0.78), 7.672 (0.41), 8.453 (0.83), 8.471 (0.82), 8.683 (3.09). |

### EXAMPLE 279

### 10-{4-[4-({(1R)-1-[3-(difluoromethyl)-2-fluorophenyl]ethyl}amino)-2-methylpyrido[3,4-d]pyrimidin-6-yl]piperazin-1-yl}-10-oxodecanoic acid

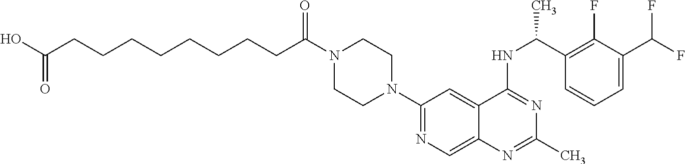 [SMILES: Cc1nc(N[C@H](C)c2cccc(C(F)F)c2F)c2cc(N3CCN(C(=O)CCCCCCCCC(=O)O)CC3)ncc2n1]

To a solution of Example 278 (195 mg, 317 μmol) in MeOH (5.5) and THF (1.5 ml) under Argon was added LiOH (1M in water, 1.9 ml). The reaction was stirred at RT for 16 h and then neutralized by the addition of 2M HCl and concentrated. The residue was purified by silica chromatography to give the title compound (185 mg, 92%).

¹H-NMR (400 MHz, DMSO-d6) δ [ppm]: 1.033 (1.69), 1.051 (3.72), 1.068 (1.55), 1.269 (4.16), 1.465 (0.73), 1.482 (1.16), 1.498 (1.22), 1.515 (1.03), 1.688 (1.77), 1.704 (1.81), 1.907 (0.63), 2.167 (1.77), 2.185 (3.41), 2.204 (1.61), 2.351 (1.10), 2.370 (1.69), 2.388 (0.99), 2.444 (1.45), 2.518 (4.86), 2.523 (3.25), 2.539 (16.00), 3.162 (0.69), 3.170 (0.76), 3.409 (0.57), 3.427 (1.00), 3.444 (0.96), 3.622 (4.67), 3.705 (0.84), 7.104 (0.81), 7.240 (1.75), 7.309 (0.49), 7.328 (1.04), 7.347 (0.59), 7.376 (0.75), 7.520 (0.41), 7.537 (0.69), 8.777 (0.80).

### EXAMPLE 280

### 4-[4-({(1R)-1-[3-(difluoromethyl)-2-fluorophenyl]ethyl}amino)-2-methylpyrido[3,4-d]pyrimidin-6-yl]-N,N-dimethylpiperazine-1-carboxamide

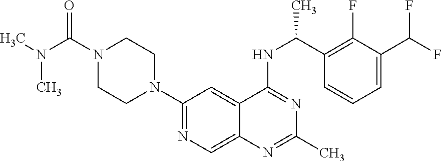 [SMILES: Cc1nc(N[C@H](C)c2cccc(C(F)F)c2F)c2cc(N3CCN(C(=O)N(C)C)CC3)ncc2n1]

To a solution of dimethylcarbamyl chloride (25.7 mg, 239 μmol) in anhydrous THF (1 ml) was added triethylamine (67 μl, 480 μmol) followed by the slow addition of Example 148 (78mg, 159 μmol). The reaction was stirred at RT for 2 h and then a few drops of water were added—The titled compound (36 mg, 45%) was isolated after preparative HPLC (basic method).

¹H-NMR (400 MHz, CHLOROFORM-d) δ [ppm]: 1.041 (0.50), 1.262 (0.58), 1.283 (0.79), 1.727 (1.41), 1.743 (1.40), 2.540 (6.00), 2.907 (16.00), 3.427 (1.04), 3.439 (1.43), 3.444 (1.12), 3.452 (1.42), 3.618 (1.24), 3.634 (1.26), 3.642 (0.81), 6.505 (0.42), 6.792 (0.45), 6.929 (0.91), 7.067 (0.43), 7.219 (0.75), 7.238 (0.43), 7.518 (0.42), 8.893 (1.48).

### EXAMPLE 281

### N-{(1R)-1-[3-(difluoromethyl)-2-fluorophenyl]ethyl}-6-[4-(methanesulfonyl)piperazin-1-yl]-2-methylpyrido[3,4-d]pyrimidin-4-amine

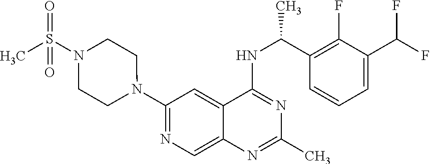 [SMILES: Cc1nc(N[C@H](C)c2cccc(C(F)F)c2F)c2cc(N3CCN(S(C)(=O)=O)CC3)ncc2n1]

To a solution of Example 148 (88 mg, 180 μmol) in DCM (1.1. ml) was added triethylamine (75 μl, 540 μmol) followed by the slow addition of methanesulfonyl chloride (30.9 mg, 270 μmol). The reaction was stirred at RT for 2 h and then a few drops of water were added—The titled compound (65 mg, 72%) was isolated after preparative HPLC (basic method).

¹H-NMR (400 MHz, CHLOROFORM-d) δ [ppm]: 1.040 (0.74), 1.194 (0.58), 1.261 (0.59), 1.282 (5.18), 1.299 (0.55), 1.730 (5.23), 1.748 (5.24), 2.543 (14.92), 2.844 (16.00), 3.403 (3.13), 3.415 (4.63), 3.427 (3.69), 3.730 (3.57), 3.743 (4.20), 3.755 (3.07), 5.773 (0.75), 5.791 (1.23), 5.809 (0.91), 5.878 (0.75), 5.895 (0.56), 6.552 (3.05), 6.783 (1.07), 6.920 (2.15), 7.058 (1.02), 7.196 (0.86), 7.216 (1.89), 7.235 (1.09), 7.497 (0.68), 7.514 (1.18), 7.533 (1.19), 7.553 (1.20), 7.571 (0.59), 8.893 (4.07).

### EXAMPLE 282

### 2-amino-1-{4-[4-({(1R)-1-[3-(difluoromethyl)-2-fluorophenyl]ethyl}amino)-2-methylpyrido[3,4-d]pyrimidin-6-yl]piperazin-1-yl}ethan-1-one

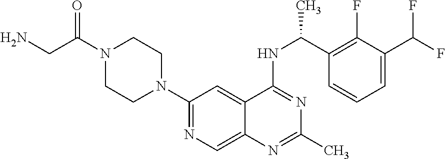 [SMILES: Cc1nc(N[C@H](C)c2cccc(C(F)F)c2F)c2cc(N3CCN(C(=O)CN)CC3)ncc2n1]

To a solution of Example 148 (1 g, 2.04 mmol) and N-Boc Glycine (537 mg, 3.07 mmol) in DMF (20 ml) under argon was added DIPEA (1.78 ml, 10.2 mmol) and HATU (1.165 g, 3.07 mmol) and stirred at RT for 16 h. The reaction was diluted with EtOAc, washed with water, sat. NaCl, dried over Na2SO4, filtered and concentrated under reduced pressure. The Boc-protected product was purified by silica chromatography (DCM:EtOH).

The Boc-protected product was treated with 4M HCl in dioxane, concentrated and a portion was purified by preparative HPLC (basic method) to give the titled compound.

¹H-NMR (400 MHz, DMSO-d6) δ [ppm]: 1.612 (5.02), 1.630 (5.00), 2.309 (16.00), 2.322 (0.79), 2.327 (0.76), 2.332 (0.56), 2.518 (2.20), 2.523 (1.50), 2.665 (0.46), 2.669 (0.65), 2.673 (0.47), 3.411 (7.92), 3.563 (2.70), 3.603 (1.56), 3.613 (1.53), 3.652 (1.25), 3.663 (1.36), 5.755 (0.76), 5.773 (1.17), 5.791 (0.76), 7.102 (1.14), 7.238 (2.50), 7.278 (0.87), 7.297 (1.88), 7.317 (1.08), 7.374 (1.01), 7.481 (2.98), 7.507 (1.10), 7.525 (0.53), 7.636 (0.59), 7.654 (1.07), 7.672 (0.54), 8.456 (1.22), 8.475 (1.16), 8.684 (4.80).

### EXAMPLE 283

### 1-{4-[4-({(1R)-1-[3-(difluoromethyl)-2-fluorophenyl]ethyl}amino)-2-methylpyrido[3,4-d]pyrimidin-6-yl]piperazin-1-yl}-2-(methylamino)ethan-1-one

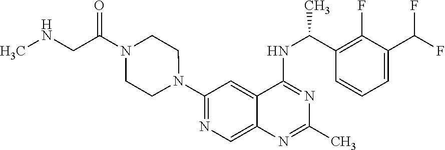 [SMILES: CNCC(=O)N1CCN(c2cc3c(N[C@H](C)c4cccc(C(F)F)c4F)nc(C)nc3cn2)CC1]

To a solution of Example 148 (1g, 2.04 mmol) and N-Boc Sarcosine (580 mg, 3.07 mmol) in DMF (20 ml) under argon was added DIPEA (1.78 ml, 10.2 mmol) and HATU (1.165 g, 3.07 mmol) and stirred at RT for 16 h. The reaction was diluted with EtOAc, washed with water, sat. NaCl, dried over Na2SO4, filtered and concentrated under reduced pressure. The Boc-protected product was purified by silica chromatography (DCM:EtOH).

The Boc-protected product was treated with 4M HCl in dioxane, concentrated and a portion was purified by preparative HPLC (basic method) to give the titled compound.

¹H-NMR (400 MHz, DMSO-d6) δ [ppm]: 1.612 (3.51), 1.630 (3.49), 2.296 (16.00), 2.310 (11.42), 2.322 (0.55), 2.327 (0.51), 2.518 (1.24), 2.523 (0.83), 2.669 (0.41), 3.385 (5.64), 3.576 (0.99), 3.608 (3.19), 3.645 (1.01), 3.658 (1.07), 5.755 (0.55), 5.773 (0.84), 5.791 (0.53), 7.102 (0.83), 7.238 (1.78), 7.278 (0.63), 7.297 (1.33), 7.317 (0.77), 7.374 (0.73), 7.482 (2.16), 7.507 (0.78), 7.636 (0.42), 7.655 (0.75), 8.456 (0.87), 8.474 (0.83), 8.684 (3.40).

### EXAMPLE 284

### 3-amino-1-{4-[4-({(1R)-1-[3-(difluoromethyl)-2-fluorophenyl]ethyl}amino)-2-methylpyrido[3,4-d]pyrimidin-6-yl]piperazin-1-yl}propan-1-one

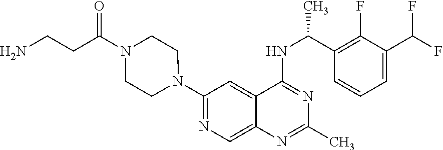 [SMILES: Cc1nc(N[C@H](C)c2cccc(C(F)F)c2F)c2cc(N3CCN(C(=O)CCN)CC3)ncc2n1]

To a solution of Example 148 (1 g, 2.04 mmol) and N-Boc g-Alanine (580 mg, 3.07 mmol) in DMF (20 ml) under argon was added DIPEA (1.78 ml, 10.2 mmol) and HATU (1.165 g, 3.07 mmol) and stirred at RT for 16 h. The reaction was diluted with EtOAc, washed with water, sat. NaCl, dried over Na2SO4, filtered and concentrated under reduced pressure. The Boc-protected product was purified by silica chromatography (DCM:EtOH).

The Boc-protected product was treated with 4M HCl in dioxane, concentrated and a portion was purified by preparative HPLC (basic method) to give the titled compound.

¹H-NMR (400 MHz, DMSO-d6) δ [ppm]: 1.613 (5.18), 1.631 (5.16), 1.751 (0.48), 2.300 (1.31), 2.310 (16.00), 2.322 (1.32), 2.327 (1.51), 2.332 (1.09), 2.336 (0.49), 2.457 (1.81), 2.473 (4.64), 2.518 (4.52), 2.523 (3.04), 2.660 (0.44), 2.665 (0.96), 2.669 (1.39), 2.673 (0.97), 2.678 (0.42), 2.757 (2.15), 2.773 (4.15), 2.789 (1.71), 3.547 (1.41), 3.630 (7.37), 3.652 (2.07), 5.756 (0.82), 5.774 (1.24), 5.792 (0.78), 7.103 (1.26), 7.238 (2.67), 7.279 (0.93), 7.298 (1.98), 7.317 (1.14), 7.374 (1.11), 7.483 (3.14), 7.508 (1.17), 7.525 (0.56), 7.637 (0.64), 7.654 (1.13), 7.672 (0.58), 8.454 (1.25), 8.472 (1.18), 8.684 (4.93).

### EXAMPLE 285

### 1-{4-[4-({(1R)-1-[3-(difluoromethyl)-2-fluorophenyl]ethyl}amino)-2-methylpyrido[3,4-d]pyrimidin-6-yl]piperazin-1-yl}-3-(methylamino)propan-1-one

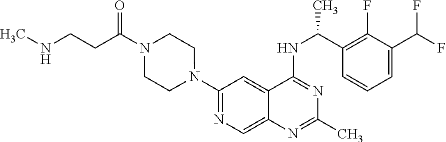 [SMILES: CNCCC(=O)N1CCN(c2cc3c(N[C@H](C)c4cccc(C(F)F)c4F)nc(C)nc3cn2)CC1]

To a solution of Example 148 (1g, 2.04 mmol) and N-(tert-butoxycarbonyl)-N-methyl-beta-alanine (623 mg, 3.07 mmol) in DMF (20 ml) under argon was added DIPEA (1.78 ml, 10.2 mmol) and HATU (1.165 g, 3.07 mmol) and stirred at RT for 16 h. The reaction was diluted with EtOAc, washed with water, sat. NaCl, dried over Na2SO4, filtered and concentrated under reduced pressure. The Boc-protected product was purified by silica chromatography (DCM:EtOH).

The Boc-protected product was treated with 4M HCl in dioxane, concentrated and a portion was purified by preparative HPLC (basic method) to give the titled compound.

¹H-NMR (400 MHz, DMSO-d6) δ [ppm]: 1.230 (0.72), 1.614 (5.39), 1.631 (5.29), 1.751 (0.92), 1.897 (0.52), 2.311 (16.00), 2.330 (14.82), 2.518 (4.60), 2.523 (2.81), 2.540 (0.68), 2.560 (1.21), 2.577 (2.64), 2.594 (1.64), 2.665 (0.88), 2.669 (1.14), 2.673 (0.83), 2.747 (1.79), 2.763 (2.96), 2.780 (1.22), 3.412 (0.85), 3.424 (0.69), 3.480 (0.43), 3.552 (1.81), 3.634 (8.43), 5.759 (3.01), 5.774 (1.33), 5.792 (0.84), 7.103 (1.28), 7.239 (2.76), 7.278 (1.03), 7.298 (2.12), 7.317 (1.21), 7.375 (1.14), 7.488 (3.84), 7.508 (1.39), 7.526 (0.65), 7.638 (0.73), 7.656 (1.28), 7.674 (0.63), 8.462 (1.37), 8.479 (1.28), 8.686 (5.28).

TABLE 10
Examples 286-289
Using the method described for Example 20: Example 10 was treated with the corresponding amines
or their hydrochloride salts and gave the desired compounds after preparative HPLC purification
(basic method) and/or silica chromatography.

| | Structure |
|---|---|
| | IUPAC-Name |
| Example | ¹H-NMR |
| 286 | 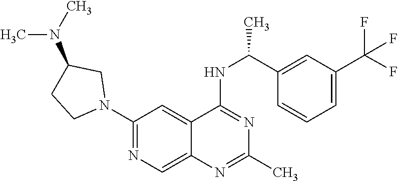 [SMILES: Cc1nc(N[C@H](C)c2cccc(C(F)(F)F)c2)c2cc(N3CC[C@@H](N(C)C)C3)ncc2n1] |
| | 6-[(3R)-3-(dimethylamino)pyrrolidin-1-yl]-2-methyl-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.107 (2.11), 1.624 (3.39), 1.642 (3.40), 2.233 |
| | (16.00), 2.318 (10.24), 2.326 (0.74), 2.332 (0.42), 2.518 (1.49), 2.523 (0.94), 2.539 (1.80), |
| | 2.669 (0.40), 3.165 (0.57), 3.186 (0.60), 3.190 (0.69), 3.211 (0.54), 3.394 (0.50), 3.411 |
| | (0.50), 3.657 (0.54), 3.732 (0.47), 3.750 (0.53), 3.757 (0.52), 3.775 (0.41), 5.623 (0.46), |
| | 5.640 (0.70), 5.659 (0.47), 7.001 (2.00), 7.565 (0.93), 7.583 (1.87), 7.586 (1.75), 7.591 |
| | (0.99), 7.735 (0.82), 7.752 (0.62), 7.799 (1.37), 8.325 (0.82), 8.345 (0.79), 8.627 (2.79). |
| 287 | 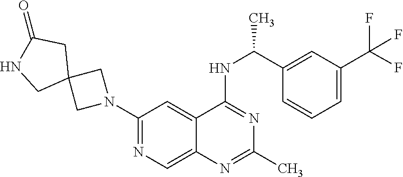 [SMILES: Cc1nc(N[C@H](C)c2cccc(C(F)(F)F)c2)c2cc(N3CC4(CNC(=O)C4)C3)ncc2n1] |
| | 2-[2-methyl-4-({(1R)-1-[3-(trifluoromethyl)phenyl]ethyl}amino)pyrido[3,4-d]pyrimidin-6- |
| | yl]-2,6-diazaspiro[3.4]octan-7-one |
| | ¹H-NMR (400 MHz, DMSO-d6) δ [ppm]: 1.608 (5.70), 1.626 (5.73), 1.920 (0.49), 2.126 |
| | (0.42), 2.330 (16.00), 2.518 (4.04), 2.523 (2.69), 2.540 (2.44), 2.549 (8.64), 2.665 (0.61), |
| | 2.669 (0.85), 2.674 (0.63), 3.528 (7.26), 3.971 (0.84), 3.995 (9.34), 4.019 (0.82), 5.603 |
| | (0.84), 5.622 (1.27), 5.639 (0.84), 7.095 (3.82), 7.547 (0.54), 7.566 (1.71), 7.585 (3.13), |
| | 7.589 (2.63), 7.608 (0.58), 7.680 (2.48), 7.731 (1.54), 7.748 (1.17), 7.796 (2.51), 8.406 |
| | (1.47), 8.425 (1.39), 8.630 (4.67). |
| 288 | 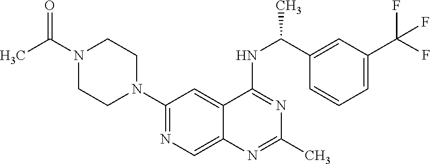 [SMILES: CC(=O)N1CCN(c2cc3c(N[C@H](C)c4cccc(C(F)(F)F)c4)nc(C)nc3cn2)CC1] |
| | 1-{4-[2-methyl-4-({(1R)-1-[3-(trifluoromethyl)phenyl]ethyl}amino)pyrido[3,4- |
| | d]pyrimidin-6-yl]piperazin-1-yl}ethan-1-one |
| | ¹H-NMR (400 MHz, DMSO-d6) δ [ppm]: 1.107 (4.74), 1.624 (5.05), 1.641 (5.10), 2.072 |
| | (16.00), 2.322 (1.04), 2.327 (1.48), 2.332 (1.43), 2.340 (15.16), 2.518 (5.25), 2.523 (3.27), |
| | 2.660 (0.42), 2.664 (0.95), 2.669 (1.39), 2.673 (0.99), 2.678 (0.44), 3.314 (0.51), 3.527 |
| | (1.37), 3.541 (1.32), 3.617 (6.16), 4.189 (0.44), 5.620 (0.71), 5.639 (1.08), 5.656 (0.71), |
| | 7.440 (2.94), 7.553 (0.46), 7.572 (1.43), 7.591 (2.69), 7.595 (2.27), 7.615 (0.49), 7.736 |
| | (1.26), 7.754 (0.97), 7.797 (2.07), 8.436 (1.21), 8.456 (1.19), 8.685 (4.46). |
| 289 | 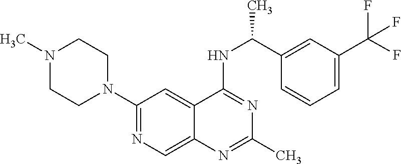 [SMILES: Cc1nc(N[C@H](C)c2cccc(C(F)(F)F)c2)c2cc(N3CCN(C)CC3)ncc2n1] |
| | 2-methyl-6-(4-methylpiperazin-1-yl)-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.616 (5.61), 1.634 (5.66), 2.247 (11.95), 2.333 |
| | (16.00), 2.459 (3.17), 2.472 (4.94), 2.522 (2.86), 2.539 (1.56), 2.665 (0.62), 2.669 (0.83), |
| | 2.673 (0.57), 3.535 (2.96), 3.547 (3.95), 3.559 (2.91), 5.613 (0.83), 5.631 (1.25), 5.649 |
| | (0.78), 7.394 (3.43), 7.550 (0.47), 7.569 (1.61), 7.587 (3.27), 7.610 (0.57), 7.733 (1.45), |
| | 7.750 (1.14), 7.793 (2.49), 8.410 (1.40), 8.429 (1.40), 8.658 (4.99). |

### EXAMPLE 290

### 6-fluoro-2-methyl-N-{(1R)-1-[2-methyl-3-(trifluoromethyl)phenyl]ethyl}pyrido[3,4-d]pyrimidin-4-amine

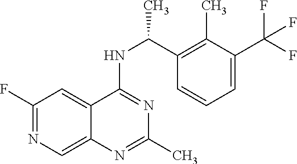 [SMILES: Cc1nc(N[C@H](C)c2cccc(C(F)(F)F)c2C)c2cc(F)ncc2n1]

Using the method described for Example 1 using 6-fluoro-2-methylpyrido[3,4-d]pyrimidin-4-ol (735 mg, 4.1 mmol) and (1R)-1-[2-methyl-3-(trifluoromethyl)phenyl]ethan-1-amine (1.00 g, 4.92 mmol) gave the titled compound (919 mg, 58%) after silica chromatography (Hexane:EtOAc).

¹H-NMR (400 MHz, DMSO-d6) δ [ppm]: 1.559 (5.53), 1.576 (5.57), 2.382 (16.00), 2.518 (2.36), 2.522 (1.58), 2.616 (6.32), 5.677 (0.83), 5.694 (1.28), 5.712 (0.83), 7.341 (0.67), 7.360 (1.52), 7.380 (0.89), 7.542 (1.63), 7.560 (1.32), 7.750 (1.49), 7.769 (1.34), 8.138 (2.47), 8.141 (2.47), 8.715 (4.00), 8.883 (1.22), 8.900 (1.19).

TABLE 11
Examples 291-295
Using the method described for Example 25: Example 10 was treated with the corresponding amines
or their hydrochloride salts and gave the desired compounds after preparative HPLC purification
(basic method) and/or silica chromatography.

| | Structure |
|---|---|
| | IUPAC-Name |
| Example | ¹H-NMR |
| 291 | 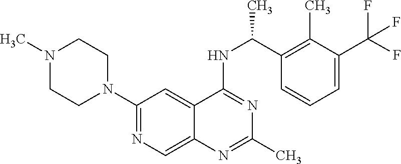 [SMILES: Cc1nc(N[C@H](C)c2cccc(C(F)(F)F)c2C)c2cc(N3CCN(C)CC3)ncc2n1] |
| | 2-methyl-6-(4-methylpiperazin-1-yl)-N-{(1R)-1-[2-methyl-3- |
| | (trifluoromethyl)phenyl]ethyl}pyrido[3,4-d]pyrimidin-4-amine |
| | ¹H-NMR (400 MHz, DMSO-d6) δ [ppm]: 1.107 (1.37), 1.558 (4.96), 1.575 (4.93), 2.254 |
| | (11.40), 2.295 (16.00), 2.323 (0.77), 2.327 (1.06), 2.332 (0.75), 2.466 (2.81), 2.479 (4.65), |
| | 2.518 (3.43), 2.523 (2.26), 2.613 (5.91), 2.665 (0.75), 2.669 (1.02), 2.674 (0.73), 3.541 |
| | (2.41), 3.554 (3.14), 3.565 (2.35), 5.679 (0.73), 5.697 (1.15), 5.714 (0.73), 7.342 (0.66), |
| | 7.361 (1.48), 7.381 (0.86), 7.428 (3.01), 7.533 (1.62), 7.552 (1.28), 7.733 (1.42), 7.753 |
| | (1.28), 8.522 (1.22), 8.540 (1.17), 8.638 (4.60). |
| 292 | 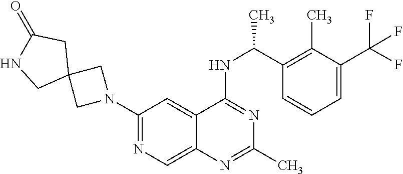 [SMILES: Cc1nc(N[C@H](C)c2cccc(C(F)(F)F)c2C)c2cc(N3CC4(CNC(=O)C4)C3)ncc2n1] |
| | 2-[2-methyl-4-({(1R)-1-[2-methyl-3-(trifluoromethyl)phenyl]ethyl}amino)pyrido[3,4- |
| | d]pyrimidin-6-yl]-2,6-diazaspiro[3.4]octan-7-one |
| | ¹H-NMR (400 MHz, DMSO-d6) δ [ppm]: 1.107 (16.00), 1.551 (2.96), 1.568 (2.96), 2.292 |
| | (9.24), 2.323 (0.44), 2.327 (0.61), 2.332 (0.43), 2.518 (2.01), 2.523 (1.33), 2.540 (0.84), |
| | 2.553 (4.69), 2.609 (3.66), 2.665 (0.44), 2.669 (0.60), 2.673 (0.41), 3.532 (3.87), 3.976 |
| | (0.50), 4.001 (4.73), 4.026 (0.48), 4.190 (1.33), 5.672 (0.46), 5.690 (0.70), 5.708 (0.45), |
| | 7.132 (2.04), 7.337 (0.44), 7.356 (0.92), 7.376 (0.53), 7.530 (1.01), 7.549 (0.80), 7.684 |
| | (1.33), 7.738 (0.90), 7.757 (0.79), 8.515 (0.76), 8.533 (0.73), 8.609 (2.65). |
| 293 | 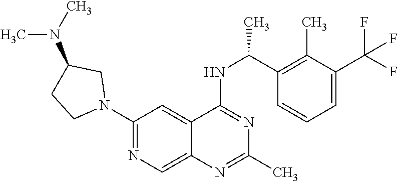 [SMILES: Cc1nc(N[C@H](C)c2cccc(C(F)(F)F)c2C)c2cc(N3CC[C@@H](N(C)C)C3)ncc2n1] |
| | 6-[(3R)-3-(dimethylamino)pyrrolidin-1-yl]-2-methyl-N-{(1R)-1-[2-methyl-3- |
| | (trifluoromethyl)phenyl]ethyl}pyrido[3,4-d]pyrimidin-4-amine |
| | ¹H-NMR (400 MHz, DMSO-d6) δ [ppm]: 1.107 (4.20), 1.563 (3.24), 1.581 (3.22), 1.874 |
| | (0.41), 2.215 (0.44),2.240(16.00), 2.279(9.87),2.518(1.80),2.523 (1.19),2.616 (4.07), |
| | 2.846 (0.41), 3.174(0.57),3.195 (0.63),3.199(0.70),3.219(0.55), 3.400(0.53), 3.417 |
| | (0.51), 3.665 (0.59),3.742(0.49), 3.759(0.56),3.767(0.55),3.784 (0.44),5.687 (0.49), |
| | 5.705 (0.76), 5.723(0.48),7.039 (2.10),7.334(0.45),7.354(1.00), 7.373(0.59), 7.526 |
| | (1.09), 7.544 (0.88), 7.747 (0.97), 7.766 (0.87), 8.439 (0.83), 8.457 (0.80), 8.606 (2.91). |
| 294 | 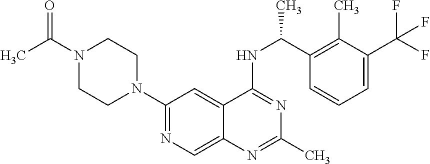 [SMILES: CC(=O)N1CCN(c2cc3c(N[C@H](C)c4cccc(C(F)(F)F)c4C)nc(C)nc3cn2)CC1] |
| | 1-{4-[2-methyl-4-({(1R)-1-[2-methyl-3-(trifluoromethyl)phenyl]ethyl}amino)pyrido[3,4- |
| | d]pyrimidin-6-yl]piperazin-1-yl}ethan-1-one |
| | ¹H-NMR (400 MHz, DMSO-d6) δ [ppm]: 0.859 (0.46), 0.967 (1.44), 1.107 (2.78), 1.143 |
| | (0.85), 1.224 (0.59), 1.565 (4.92), 1.582 (4.95), 2.076 (16.00), 2.303 (15.17), 2.518 (9.17), |
| | 2.522 (6.33), 2.613 (6.33), 3.535 (1.64), 3.545 (1.47), 3.622 (6.37), 5.684 (0.77), 5.702 |
| | (1.18), 5.719 (0.74), 7.343 (0.70), 7.363 (1.58), 7.383 (0.92), 7.473 (3.09), 7.536 (1.69), |
| | 7.555 (1.38), 7.735 (1.49), 7.754 (1.38), 8.548 (1.27), 8.565 (1.23), 8.665 (4.51). |
| 295 | 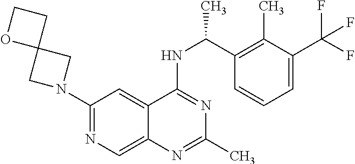 [SMILES: Cc1nc(N[C@H](C)c2cccc(C(F)(F)F)c2C)c2cc(N3CC4(CCO4)C3)ncc2n1] |
| | 2-methyl-N-{(1R)-1-[2-methyl-3-(trifluoromethyl)phenyl]ethyl}-6-(1-oxa-6- |
| | azaspiro[3.3]heptan-6-yl)pyrido[3,4-d]pyrimidin-4-amine |
| | ¹H-NMR (400 MHz, DMSO-d6) δ [ppm]: 1.231 (0.96), 1.627 (8.83), 1.644 (8.87), 2.453 |
| | (16.00), 2.518 (5.84), 2.523 (4.29), 2.584 (12.16), 2.679 (0.49), 2.905 (2.63), 2.924 (5.71), |
| | 2.942 (2.75), 3.442 (0.73), 4.144 (2.50), 4.153 (2.41), 4.155 (2.51), 4.169 (3.17), 4.178 |
| | (3.09), 4.180 (3.12), 4.319 (3.02), 4.328 (3.06), 4.344 (2.40), 4.352 (2.37), 4.463 (3.13), |
| | 4.482 (6.36), 4.501 (3.01), 5.810 (1.14), 5.827 (1.74), 5.844 (1.15), 7.317 (4.47), 7.397 |
| | (1.33), 7.417 (2.89), 7.437 (1.71), 7.592 (3.20), 7.610 (2.58), 7.777 (2.95), 7.796 (2.64), |
| | 8.693 (8.01). |

### EXAMPLE 296

### 6-fluoro-2,8-dimethyl-N-{(1R)-1-[3-(trifluoromethyl)phenyl]ethyl}pyrido[3,4-d]pyrimidin-4-amine

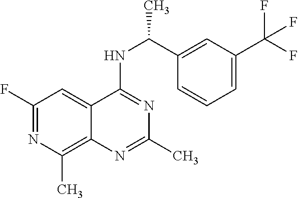 [SMILES: Cc1nc(N[C@H](C)c2cccc(C(F)(F)F)c2)c2cc(F)nc(C)c2n1]

To a solution of Example 10 (250 mg, 714 μmol) in DMSO (5 ml) was added DBU (213 μl, 1.4 mmol) and nitromethane (193 μl, 3.6 mmol) and stirred for 4 days at RT. The reaction was diluted with water and the solid collected by filtration and washed with water. The solid was dried to give the title compound (260 mg, 95%).

¹H-NMR (400 MHz, DMSO-d6) δ [ppm]: 1.153 (0.82), 1.171 (1.59), 1.189 (0.82), 1.603 (5.61), 1.621 (5.61), 1.986 (3.24), 2.429 (16.00), 2.518 (1.22), 2.523 (0.74), 2.539 (1.06), 2.724 (11.33), 4.016 (0.71), 4.034 (0.70), 5.592 (0.75), 5.609 (1.14), 5.628 (0.74), 5.758 (2.79), 7.544 (0.46), 7.563 (1.48), 7.582 (1.74), 7.591 (1.88), 7.611 (0.55), 7.740 (1.27), 7.758 (1.00), 7.818 (2.04), 7.889 (2.05), 7.892 (2.07), 8.639 (1.19), 8.658 (1.16).

TABLE 12
Examples 297-300
Using the method described for Example 25: Example 296 was treated with the corresponding
amines or their hydrochloride salts and gave the desired compounds after preparative HPLC
purification (basic method) and/or silica chromatography.

| | Structure |
|---|---|
| | IUPAC-Name |
| Example | ¹H-NMR |
| 297 | 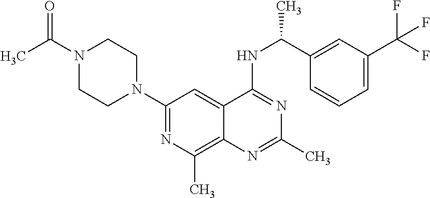 [SMILES: CC(=O)N1CCN(c2cc3c(N[C@H](C)c4cccc(C(F)(F)F)c4)nc(C)nc3c(C)n2)CC1] |
| | 1-{4-[2,8-dimethyl-4-({(1R)-1-[3-(trifluoromethyl)phenyl]ethyl}amino)pyrido[3,4- |
| | d]pyrimidin-6-yl]piperazin-1-yl}ethan-1-one |
| | ¹H-NMR (400 MHz, DMSO-d6) δ [ppm]: 1.616 (5.38), 1.634 (5.38), 2.069 (16.00), 2.323 |
| | (0.79), 2.327 (1.14), 2.332 (0.87), 2.336 (0.50), 2.352 (15.44), 2.518 (4.53), 2.523 (2.86), |
| | 2.662 (14.26), 2.673 (1.14), 3.504 (1.68), 3.518 (1.47), 3.572 (0.41), 3.598 (9.27), 3.617 |
| | (2.19), 5.611 (0.77), 5.628 (1.16), 5.647 (0.77), 7.249 (3.17), 7.547 (0.48), 7.566 (1.57), |
| | 7.584 (3.08), 7.588 (2.61), 7.608 (0.50), 7.728 (1.41), 7.746 (1.06), 7.789 (2.32), 8.326 |
| | (1.35), 8.345 (1.28). |
| 298 | 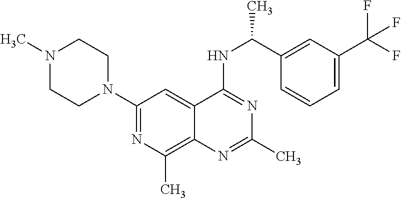 [SMILES: Cc1nc(N[C@H](C)c2cccc(C(F)(F)F)c2)c2cc(N3CCN(C)CC3)nc(C)c2n1] |
| | 2,8-dimethyl-6-(4-methylpiperazin-1-yl)-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.107 (0.69), 1.609 (5.29), 1.627 (5.29), 2.246 |
| | (11.02), 2.323 (0.82), 2.327 (1.20), 2.332 (0.94), 2.337 (0.82), 2.345 (16.00), 2.451 (2.52), |
| | 2.463 (3.84), 2.476 (3.28), 2.518 (3.72), 2.523 (2.52), 2.645 (13.61), 2.660 (0.50), 2.665 |
| | (0.82), 2.669 (1.13), 2.673 (0.76), 3.298 (0.38), 3.514 (2.33), 3.526 (3.09), 3.538 (2.27), |
| | 5.604 (0.69), 5.622 (1.07), 5.640 (0.69), 7.199 (3.02), 7.544 (0.44), 7.563 (1.45), 7.581 |
| | (2.96), 7.585 (2.58), 7.604 (0.44), 7.725 (1.32), 7.742 (0.94), 7.786 (2.14), 8.297 (1.26), |
| | 8.316 (1.20). |
| 299 | 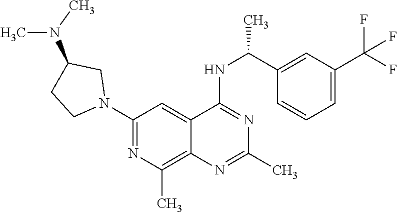 [SMILES: Cc1nc(N[C@H](C)c2cccc(C(F)(F)F)c2)c2cc(N3CC[C@@H](N(C)C)C3)nc(C)c2n1] |
| | 6-[(3R)-3-(dimethylamino)pyrrolidin-1-yl]-2,8-dimethyl-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.107 (2.10), 1.617 (3.57), 1.635 (3.61), 1.847 |
| | (0.47), 1.871 (0.40), 2.176 (0.46), 2.190 (0.47), 2.207 (0.47), 2.233 (16.00), 2.330 (10.04), |
| | 2.643 (8.79), 2.665 (0.80), 2.669 (0.99), 2.673 (0.71), 2.824 (0.50), 3.140 (0.61), 3.165 |
| | (0.79), 3.186 (0.59), 3.374 (0.71), 3.391 (0.65), 3.399 (0.50), 3.655 (0.67), 3.730 (0.52), |
| | 3.747 (0.62), 3.772 (0.47), 5.613 (0.52), 5.632 (0.79), 5.651 (0.52), 6.815 (2.22), 7.559 |
| | (1.01), 7.579 (2.27), 7.727 (0.92), 7.745 (0.74), 7.790 (1.64), 8.205 (0.92), 8.225 (0.92). |
| 300 | 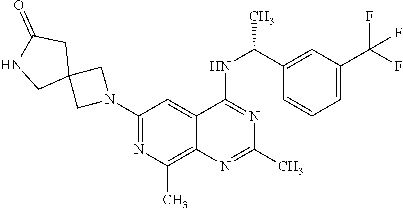 [SMILES: Cc1nc(N[C@H](C)c2cccc(C(F)(F)F)c2)c2cc(N3CC4(CNC(=O)C4)C3)nc(C)c2n1] |
| | 2-[2,8-dimethyl-4-({(1R)-1-[3-(trifluoromethyl)phenyl]ethyl}amino)pyrido[3,4- |
| | d]pyrimidin-6-yl]-2,6-diazaspiro[3.4]octan-7-one |
| | ¹H-NMR (400 MHz, DMSO-d6) 8 [ppm]: 1.107 (3.78), 1.600 (5.71), 1.617 (5.81), 2.323 |
| | (1.21), 2.327 (1.68), 2.331 (1.61), 2.342 (16.00), 2.522 (7.39), 2.537 (10.19), 2.635 (14.20), |
| | 2.665 (1.17), 2.669 (1.52), 2.673 (1.12), 3.518 (7.25), 3.946 (0.86), 3.970 (9.33), 3.994 |
| | (0.98), 5.593 (0.84), 5.611 (1.28), 5.630 (0.86), 6.916 (3.87), 7.541 (0.49), 7.560 (1.68), |
| | 7.578 (3.38), 7.582 (2.92), 7.601 (0.65), 7.671 (2.64), 7.722 (1.54), 7.740 (1.24), 7.788 |
| | (2.64), 8.287 (1.49), 8.306 (1.47). |

### EXAMPLE 301

### 6-fluoro-2,8-dimethyl-N-{(1R)-1-[2-methyl-3-(trifluoromethyl)phenyl]ethyl}pyrido[3,4-d]pyrimidin-4-amine

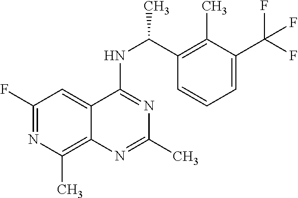 [SMILES: Cc1nc(N[C@H](C)c2cccc(C(F)(F)F)c2C)c2cc(F)nc(C)c2n1]

To a solution of Example 290 (250 mg, 868 μmol) in DMSO (4.8 ml) was added DBU (205 μl, 1.4 mmol) and nitromethane (186 μl, 3.4 mmol) and stirred for 4 days at RT. The reaction was diluted with water and the solid collected by filtration and washed with water. The solid was dried to give the title compound (243 mg, 89%).

¹H-NMR (400 MHz, DMSO-d6) δ [ppm]: 1.550 (5.96), 1.567 (6.02), 1.987 (0.41), 2.327 (0.61), 2.389 (16.00), 2.539 (4.42), 2.615 (7.78), 2.669 (0.66), 2.708 (12.68), 5.665 (0.92), 5.683 (1.45), 5.700 (0.92), 7.333 (0.80), 7.353 (1.78), 7.372 (1.06), 7.535 (1.93), 7.555 (1.60), 7.739 (1.77), 7.759 (1.61), 7.932 (2.66), 8.760 (1.45), 8.777 (1.42).

TABLE 13
Examples 302-307
Using the method described for Example 25: Example 301 was treated with the corresponding
amines or their hydrochloride salts and gave the desired compounds after preparative HPLC
purification (basic method) and/or silica chromatography.

| | Structure |
|---|---|
| | IUPAC-Name |
| Example | ¹H-NMR |
| 302 | 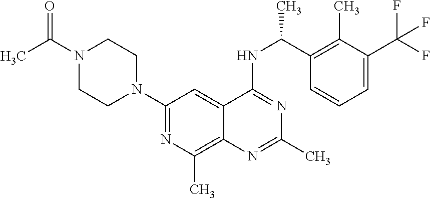 [SMILES: CC(=O)N1CCN(c2cc3c(N[C@H](C)c4cccc(C(F)(F)F)c4C)nc(C)nc3c(C)n2)CC1] |
| | 1-{4-[2,8-dimethyl-4-({(1R)-1-[2-methyl-3- |
| | (trifluoromethyl)phenyl]ethyl}amino)pyrido[3,4-d]pyrimidin-6-yl]piperazin-1-yl}ethan-1- |
| | one |
| | ¹H-NMR (400 MHz, DMSO-d6) δ [ppm]: 0.967 (0.40), 1.109 (0.43), 1.224 (0.49), 1.556 |
| | (5.06), 1.574 (4.97), 2.074 (16.00), 2.314 (15.91), 2.321 (1.32), 2.326 (1.17), 2.331 (0.75), |
| | 2.518 (3.32), 2.522 (2.15), 2.612 (6.32), 2.643 (14.12), 2.660 (0.43), 2.664 (0.75), 2.668 |
| | (0.97), 2.673 (0.68), 3.510 (1.72), 3.522 (1.38), 3.575 (0.49), 3.604 (6.91), 3.623 (2.09), |
| | 5.675 (0.77), 5.692 (1.18), 5.710 (0.77), 7.283 (3.09), 7.336 (0.72), 7.355 (1.57), 7.374 |
| | (0.92), 7.529 (1.71), 7.547 (1.37), 7.729 (1.52), 7.748 (1.37), 8.433 (1.29), 8.451 (1.26). |
| 303 | 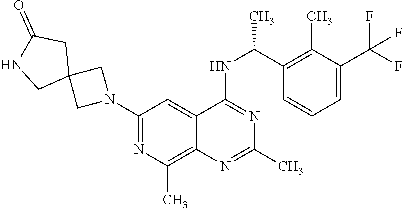 [SMILES: Cc1nc(N[C@H](C)c2cccc(C(F)(F)F)c2C)c2cc(N3CC4(CNC(=O)C4)C3)nc(C)c2n1] |
| | 2-[2,8-dimethyl-4-({(1R)-1-[2-methyl-3-(trifluoromethyl)phenyl]ethyl}amino)pyrido[3,4- |
| | d]pyrimidin-6-yl]-2,6-diazaspiro[3.4]octan-7-one |
| | ¹H-NMR (400 MHz, DMSO-d6) δ [ppm]: 0.967 (1.28), 1.107 (14.01), 1.144 (0.80), 1.542 |
| | (3.02), 1.559 (3.05), 2.303 (9.63), 2.322 (0.52), 2.326 (0.68), 2.331 (0.47), 2.518 (2.23), |
| | 2.522 (1.52), 2.539 (16.00), 2.616 (9.32), 2.664 (0.49), 2.668 (0.65), 2.673 (0.47), 3.521 |
| | (3.96), 3.951 (0.57), 3.976 (4.61), 4.001 (0.53), 4.188 (1.22), 5.662 (0.47), 5.679 (0.72), |
| | 5.697 (0.47), 6.955 (2.15), 7.328 (0.43), 7.348 (0.94), 7.367 (0.55), 7.523 (1.03), 7.541 |
| | (0.83), 7.675 (1.43), 7.732 (0.92), 7.751 (0.83), 8.395 (0.79), 8.413 (0.77). |
| 304 | 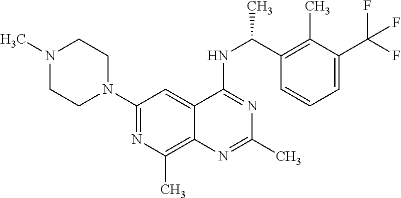 [SMILES: Cc1nc(N[C@H](C)c2cccc(C(F)(F)F)c2C)c2cc(N3CCN(C)CC3)nc(C)c2n1] |
| | 2,8-dimethyl-6-(4-methylpiperazin-1-yl)-N-{(1R)-1-[2-methyl-3- |
| | (trifluoromethyl)phenyl]ethyl}pyrido[3,4-d]pyrimidin-4-amine |
| | ¹H-NMR (400 MHz, DMSO-d6) δ [ppm]: 1.549 (2.46), 1.567 (2.44), 2.252 (5.47), 2.307 |
| | (7.82), 2.327 (0.44), 2.458 (1.30), 2.470 (2.04), 2.518 (1.56), 2.523 (1.05), 2.540 (16.00), |
| | 2.614 (3.15), 2.627 (6.92), 2.669 (0.42), 3.521 (1.18), 3.533 (1.57), 3.544 (1.16), 5.687 |
| | (0.57), 7.235 (1.52), 7.354 (0.76), 7.373 (0.44), 7.527 (0.82), 7.544 (0.66), 7.729 (0.73), |
| | 7.748 (0.66), 8.409 (0.62), 8.426 (0.61). |
| 305 | 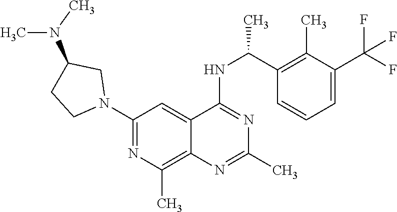 [SMILES: Cc1nc(N[C@H](C)c2cccc(C(F)(F)F)c2C)c2cc(N3CC[C@@H](N(C)C)C3)nc(C)c2n1] |
| | 6-[(3R)-3-(dimethylamino)pyrrolidin-1-yl]-2,8-dimethyl-N-{(1R)-1-[2-methyl-3- |
| | (trifluoromethyl)phenyl]ethyl}pyrido[3,4-d]pyrimidin-4-amine |
| | ¹H-NMR (400 MHz, DMSO-d6) δ [ppm]: 1.555 (3.48), 1.572 (3.53), 1.853 (0.45), 1.906 |
| | (0.95), 2.183 (0.41), 2.196 (0.41), 2.208 (0.41), 2.240 (16.00), 2.290 (10.67), 2.518 (3.34), |
| | 2.523 (2.21), 2.623 (11.07), 2.831 (0.41), 3.150 (0.59), 3.171 (0.68), 3.175 (0.77), 3.195 |
| | (0.59), 3.295 (0.50), 3.378 (1.45), 3.396 (0.90), 3.403 (0.63), 3.662 (0.59), 3.739 (0.50), |
| | 3.757 (0.59), 3.764 (0.59), 3.782 (0.45), 5.677 (0.54), 5.694 (0.81), 5.711 (0.54), 6.853 |
| | (2.26), 7.325 (0.50), 7.345 (1.08), 7.364 (0.63), 7.518 (1.18), 7.537 (0.95), 7.740 (1.08), |
| | 7.759 (0.95), 8.319 (0.90), 8.336 (0.86). |
| 306 | 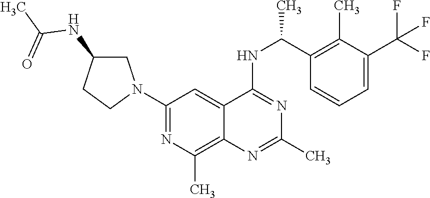 [SMILES: CC(=O)N[C@@H]1CCN(c2cc3c(N[C@H](C)c4cccc(C(F)(F)F)c4C)nc(C)nc3c(C)n2)C1] |
| | N-{(3R)-1-[2,8-dimethyl-4-({(1R)-1-[2-methyl-3- |
| | (trifluoromethyl)phenyl]ethyl}amino)pyrido[3,4-d]pyrimidin-6-yl]pyrrolidin-3- |
| | yl}acetamide |
| | ¹H-NMR (400 MHz, DMSO-d6) δ [ppm]: 0.967 (0.48), 1.547 (4.51), 1.565 (4.57), 1.824 |
| | (16.00), 1.915 (0.54), 1.928 (0.60), 1.947 (0.42), 2.167 (0.48), 2.182 (0.60), 2.199 (0.54), |
| | 2.214 (0.42), 2.294 (13.77), 2.518 (6.98), 2.522 (4.39), 2.619 (6.80), 2.625 (13.65), 3.281 |
| | (0.42), 3.289 (0.84), 3.299 (1.02), 3.309 (0.90), 3.379 (0.60),3.502 (0.54), 3.515 (0.66), |
| | 3.587 (0.42), 3.605 (0.84), 3.623 (0.48), 3.630 (0.66), 3.655 (0.84), 3.671 (0.96), 3.682 |
| | (0.78), 3.698 (0.72), 4.368 (0.66), 4.380 (0.66), 5.676 (0.72), 5.694 (1.08), 5.711 (0.72), |
| | 6.886 (2.89), 7.331 (0.66), 7.350 (1.44), 7.370 (0.84), 7.521 (1.56), 7.538 (1.26), 7.734 |
| | (1.38), 7.752 (1.26), 8.173 (1.20), 8.189 (1.20), 8.371 (1.20), 8.389 (1.14). |
| 307 | 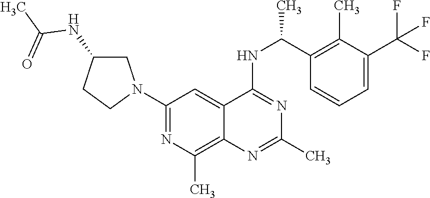 [SMILES: CC(=O)N[C@H]1CCN(c2cc3c(N[C@H](C)c4cccc(C(F)(F)F)c4C)nc(C)nc3c(C)n2)C1] |
| | N-{(3S)-1-[2,8-dimethyl-4-({(1R)-1-[2-methyl-3- |
| | (trifluoromethyl)phenyl]ethyl}amino)pyrido[3,4-d]pyrimidin-6-yl]pyrrolidin-3- |
| | yl}acetamide |
| | ¹H-NMR (400 MHz, DMSO-d6) δ [ppm]: 1.224 (0.58), 1.549 (4.68), 1.566 (4.75), 1.829 |
| | (16.00), 1.912 (0.64), 1.926 (0.67), 1.943 (0.48), 2.162 (0.51), 2.179 (0.61), 2.193 (0.55), |
| | 2.209 (0.42), 2.293 (13.76), 2.332 (1.38), 2.518 (9.88), 2.522 (6.16), 2.626 (14.30), 2.673 |
| | (1.38), 3.300 (1.06), 3.532 (0.61), 3.546 (0.71), 3.566 (0.58), 3.589 (0.99), 3.607 (0.55), |
| | 3.615 (0.58), 3.633 (0.99), 3.649 (0.96), 3.660 (0.83), 3.675 (0.74), 4.363 (0.42), 4.378 |
| | (0.67), 4.390 (0.67), 5.672 (0.74), 5.690 (1.12), 5.707 (0.71), 6.880 (3.01), 7.329 (0.71), |
| | 7.349 (1.51), 7.368 (0.90), 7.520 (1.67), 7.538 (1.35), 7.732 (1.47), 7.751 (1.31), 8.178 |
| | (1.31), 8.195 (1.31), 8.370 (1.25), 8.388 (1.19). |

### EXAMPLE 308

### 6-chloro-2-methyl-N-{(1R)-1-[3-(trifluoromethyl)phenyl]ethyl}pyrido[3,4-d]pyrimidin-4-amine

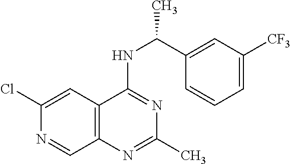 [SMILES: Cc1nc(N[C@H](C)c2cccc(C(F)(F)F)c2)c2cc(Cl)ncc2n1]

To a solution of Intermediate 15 (2.00 g, 9.71 mmol) in DMF (40 ml) were added triethylamine (4.7 ml, 34 mmol), 4-(dimethylamino)pyridine (1 crystal) and 2,4,6-tri(propan-2-yl)benzene-1-sulfonyl chloride (3.24 g, 10.7 mmol) at RT. The reaction mixture was stirred at RT for 1 hour. Then (1R)-1-[3-(trifluoromethyl)phenyl]ethan-1-amine hydrochloride (2.66 g, 11.7 mmol) was added and stirred at room temperature for 16 hours. The reaction mixture was diluted with water and extracted with EtOAc. The organic phase was washed with water and brine, dried over anhydrous sodium sulfate and concentrated to give a residue. The residue was purified by silica gel column chromatography (petroleum ether: ethyl acetate to give the titled compound (3.2 g, 84%).

¹H-NMR (400 MHz, DMSO-d6) δ [ppm]: 1.605 (5.96), 1.622 (5.97), 2.423 (16.00), 2.518 (1.39), 2.523 (0.89), 5.589 (0.71), 5.607 (1.08), 5.625 (0.70), 7.548 (0.50), 7.567 (1.59), 7.586 (1.75), 7.597 (1.94), 7.617 (0.61), 7.749 (1.40), 7.767 (1.11), 7.825 (2.30), 8.481 (4.08), 8.831 (4.99), 8.849 (1.11).

TABLE 14
Examples 309-314
Using the general method: To a solution of Example 308 (100 mg, 263 μmol) in tetrahydrofuran (1.9
ml) was added the boronic acid or pinacol borate ester (1.2 eq), potassium phosphate (2M in water,
2 eq) and methanesulfonato(2-dicyclohexylphosphino-2′,4′,6′-tri-i-propyl-1,1′-biphenyl)(2′-amino-
1,1′-biphenyl-2-yl)palladium(II) (0.1 eq) at RT. The reaction mixture was stirred at 70° C. for 16 hours
under a nitrogen atmosphere. The reaction was diluted with water and extracted with EtOAc. The
desired compounds were isolated after preparative HPLC purification (basic method) and/or silica
chromatography.

| | Structure |
|---|---|
| | IUPAC-Name |
| Example | ¹H-NMR |
| 309 | 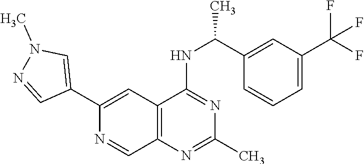 [SMILES: Cc1nc(N[C@H](C)c2cccc(C(F)(F)F)c2)c2cc(-c3cnn(C)c3)ncc2n1] |
| | 2-methyl-6-(1-methyl-1H-pyrazol-4-yl)-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.672 (6.23), 1.690 (6.22), 2.435 (14.31), 3.922 |
| | (16.00), 5.666 (0.91), 5.684 (1.35), 5.701 (0.88), 7.563 (0.59), 7.582 (1.92), 7.601 (3.48), |
| | 7.625 (0.68), 7.794 (1.71), 7.812 (1.40), 7.850 (2.97), 8.040 (5.51), 8.134 (0.84), 8.253 |
| | (5.23), 8.574 (3.02), 8.947 (5.40). |
| 310 | 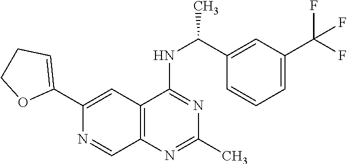 [SMILES: Cc1nc(N[C@H](C)c2cccc(C(F)(F)F)c2)c2cc(C3=CCCO3)ncc2n1] |
| | 6-(4,5-dihydrofuran-2-yl)-2-methyl-N-{(lR)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.222 (0.69), 1.606 (6.00), 1.624 (6.11), 1.637 |
| | (0.80), 1.655 (0.46), 2.070 (0.69), 2.419 (16.00), 2.462 (0.80), 2.827 (1.03), 2.833 (1.03), |
| | 2.850 (2.06), 2.857 (2.06), 2.873 (1.09), 2.880 (1.03), 4.546 (2.00), 4.569 (4.11), 4.593 |
| | (1.89), 5.609 (0.91), 5.627 (1.37), 5.646 (0.91), 5.878 (1.37), 5.885 (3.03), 5.892 (1.43), |
| | 7.541 (0.51), 7.561 (1.77), 7.579 (3.09), 7.584 (2.86), 7.604 (0.80), 7.751 (1.54), 7.769 |
| | (1.37), 7.831 (2.69), 8.303 (4.11), 8.918 (4.57), 8.953 (1.49), 8.963 (0.74), 8.972 (1.54). |
| 311 | 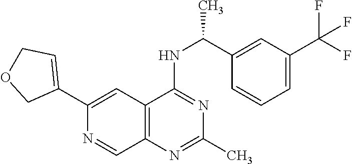 [SMILES: Cc1nc(N[C@H](C)c2cccc(C(F)(F)F)c2)c2cc(C3=CCOC3)ncc2n1] |
| | 6-(2,5-dihydrofuran-3-yl)-2-methyl-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.643 (5.98), 1.661 (5.96), 2.327 (0.60), 2.422 |
| | (16.00), 2.669 (0.41), 4.824 (2.66), 5.039 (2.35), 5.641 (0.89), 5.659 (1.33), 5.677 (0.90), |
| | 6.755 (2.31), 7.558 (0.59), 7.577 (1.79), 7.596 (2.91), 7.602 (2.83), 7.622 (0.81), 7.757 |
| | (1.60), 7.775 (1.37), 7.819 (2.68), 8.291 (3.68), 8.401 (0.46), 8.755 (1.38), 8.774 (1.39), |
| | 8.951 (4.51). |
| 312 | 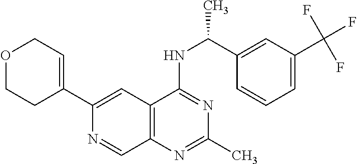 [SMILES: Cc1nc(N[C@H](C)c2cccc(C(F)(F)F)c2)c2cc(C3=CCOCC3)ncc2n1] |
| | 6-(3,6-dihydro-2H-pyran-4-yl)-2-methyl-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.638 (6.31), 1.655 (6.27), 2.416 (16.00), 2.632 |
| | (1.95), 2.669 (0.54), 3.875 (2.01), 3.888 (4.04), 3.902 (1.96), 4.326 (3.58), 4.333 (3.62), |
| | 5.644 (0.93), 5.662 (1.41), 5.680 (0.92), 6.905 (2.15), 7.553 (0.51), 7.573 (1.82), 7.591 |
| | (3.47), 7.615 (0.72), 7.756 (1.67), 7.773 (1.37), 7.816 (2.93), 8.271 (4.01), 8.778 (1.48), |
| | 8.796 (1.45), 8.941 (5.09). |
| 313 | 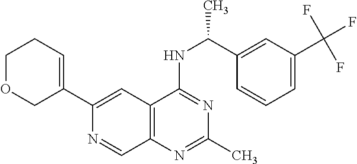 [SMILES: Cc1nc(N[C@H](C)c2cccc(C(F)(F)F)c2)c2cc(C3=CCCOC3)ncc2n1] |
| | 6-(5,6-dihydro-2H-pyran-3-yl)-2-methyl-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.633 (6.00), 1.651 (6.01), 2.347 (1.87), 2.357 |
| | (1.89), 2.376 (1.51), 2.410 (16.00), 3.751 (0.40), 3.771 (1.99), 3.784 (4.06), 3.798 (1.91), |
| | 4.565 (0.48), 4.570 (0.49), 4.605 (1.74), 4.611 (2.18), 4.617 (2.21), 4.622 (1.79), 5.633 |
| | (0.86), 5.651 (1.32), 5.669 (0.86), 6.905 (0.86), 6.916 (1.76), 6.925 (0.97), 7.554 (0.51), |
| | 7.573 (1.70), 7.592 (3.01), 7.597 (2.71), 7.616 (0.64), 7.752 (1.53), 7.770 (1.23), 7.815 |
| | (2.63), 8.279 (3.86), 8.731 (1.42), 8.750 (1.39), 8.915 (4.74). |
| 314 | 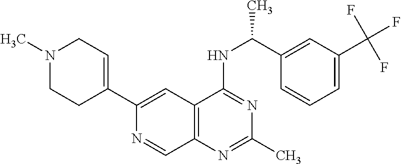 [SMILES: Cc1nc(N[C@H](C)c2cccc(C(F)(F)F)c2)c2cc(C3=CCN(C)CC3)ncc2n1] |
| | 2-methyl-6-(1-methyl-1,2,3,6-tetrahydropyridin-4-yl)-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.051 (0.51), 1.220 (1.54), 1.632 (5.99), 1.650 |
| | (5.90), 1.982 (0.43), 2.311 (12.15), 2.407 (16.00), 2.434 (0.43), 2.626 (2.91), 2.638 (2.82), |
| | 3.111 (2.65), 4.369 (0.43), 5.635 (0.86), 5.653 (1.37), 5.671 (0.86), 6.839 (1.88), 7.550 |
| | (0.51), 7.570 (1.71), 7.588 (3.34), 7.592 (2.91), 7.612 (0.68), 7.750 (1.54), 7.768 (1.20), |
| | 7.812 (2.65), 8.249 (3.59), 8.756 (1.45), 8.775 (1.37), 8.919 (4.71). |

TABLE 15
Examples 315-318
Following the method described here for Example 315, the Examples in Table 14 were used to
prepare their corresponding analogs in Table 15.
Example 315: To a solution of Example 311 (180 mg, 445 pmol) in MeOH (4 ml) was added palladium
on activated charcoal (10%, 0.1 eq). The reaction vessel was flushed with hydrogen and stirred for 4 h
at RT. The reaction was filtered through Celite, and the filtrate was concentrated. The desired
compounds were isolated after preparative HPLC purification (acidic or basic method) and/or by
silica chromatography.

| | Structure |
|---|---|
| | IUPAC-Name |
| Example | ¹H-NMR |
| 315 | 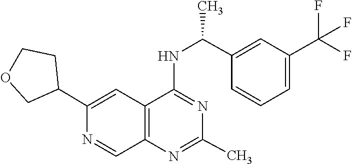 [SMILES: Cc1nc(N[C@H](C)c2cccc(C(F)(F)F)c2)c2cc(C3CCOC3)ncc2n1] |
| | 2-methyl-6-[(3RS)-oxolan-3-yl]-N-{(1R)-1-[3-(trifluoromethyl)phenyl]ethyl}pyrido[3,4- |
| | d]pyrimidin-4-amine amine (mixture of stereoisomers) |
| | ¹H-NMR (400 MHz, DMSO-d6) δ [ppm]: 1.621 (6.28), 1.639 (6.39), 2.182 (0.51), 2.189 |
| | (0.60), 2.201 (0.79), 2.211 (0.94), 2.220 (0.90), 2.232 (0.86), 2.239 (0.76), 2.251 (0.42), |
| | 2.323 (1.01), 2.336 (1.13), 2.345 (0.80), 2.354 (0.92), 2.366 (0.61), 2.411 (16.00), 2.669 |
| | (0.46), 3.642 (1.20), 3.662 (1.82), 3.682 (1.37), 3.702 (0.50), 3.760 (1.11), 3.766 (1.12), |
| | 3.780 (1.80), 3.786 (1.75), 3.800 (0.96), 3.806 (0.89), 3.841 (0.64), 3.860 (1.75), 3.879 |
| | (2.00), 3.898 (0.87), 3.941 (0.83), 3.954 (1.04), 3.962 (1.39), 3.974 (1.37), 3.995 (0.55), |
| | 4.133 (1.39), 4.153 (2.50), 4.173 (1.21), 5.622 (0.97), 5.640 (1.46), 5.658 (0.96), 7.548 |
| | (0.62), 7.567 (1.92), 7.586 (3.32), 7.591 (2.94), 7.611 (0.80), 7.752 (1.78), 7.770 (1.43), |
| | 7.824 (2.85), 8.158 (4.18), 8.365 (0.43), 8.715 (1.52), 8.734 (1.45), 8.950 (4.62). |
| 316 | 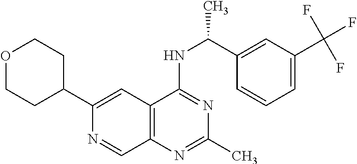 [SMILES: Cc1nc(N[C@H](C)c2cccc(C(F)(F)F)c2)c2cc(C3CCOCC3)ncc2n1] |
| | 2-methyl-6-(oxan-4-yl)-N-{(1R)-1-[3-(trifluoromethyl)phenyl]ethyl}pyrido[3,4- |
| | d]pyrimidin-4-amine |
| | ¹H-NMR (400 MHz, DMSO-d6) δ [ppm]: 1.624 (6.16), 1.642 (6.15), 1.810 (0.65), 1.820 |
| | (0.98), 1.839 (1.14), 1.849 (1.99), 1.866 (3.05), 1.874 (2.90), 2.409 (16.00), 3.006 (0.62), |
| | 3.018 (0.67), 3.033 (1.00), 3.044 (0.74), 3.060 (0.50), 3.458 (1.78), 3.467 (1.79), 3.487 |
| | (2.50), 3.494 (2.72), 3.515 (1.39), 3.522 (1.40), 3.985 (2.03), 4.012 (1.64), 4.019 (1.53), |
| | 5.627 (0.92), 5.645 (1.39), 5.663 (0.90), 7.550 (0.52), 7.569 (1.79), 7.587 (3.46), 7.612 |
| | (0.65), 7.752 (1.63), 7.770 (1.31), 7.822 (2.83), 8.125 (4.24), 8.684 (1.48), 8.703 (1.48), |
| | 8.931 (4.98). |
| 317 | 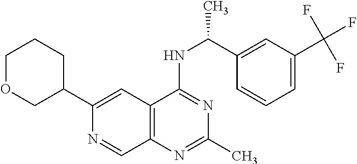 [SMILES: Cc1nc(N[C@H](C)c2cccc(C(F)(F)F)c2)c2cc(C3CCCOC3)ncc2n1] |
| | 2-methyl-6-[(3RS)-oxan-3-yl]-N-{(1R)-1-[3-(trifluoromethyl)phenyl]ethyl}pyrido[3,4- |
| | d]pyrimidin-4-amine (mixture of stereoisomers) |
| | ¹H-NMR (400 MHz, DMSO-d6) δ [ppm]: 1.626 (6.00), 1.643 (5.97), 1.705 (1.89), 1.896 |
| | (0.54), 1.927 (0.66), 1.937 (0.58), 2.081 (0.90), 2.111 (0.67), 2.327 (0.99), 2.409 (16.00), |
| | 2.669 (0.49), 3.011 (0.66), 3.029 (0.73), 3.039 (1.08), 3.066 (0.69), 3.420 (0.90), 3.479 |
| | (1.16), 3.506 (1.98), 3.532 (1.04), 3.905 (1.04), 3.932 (0.91), 4.042 (0.88), 4.070 (0.79), |
| | 5.626 (0.92), 5.644 (1.35), 5.662 (0.87), 7.553 (0.60), 7.572 (1.78), 7.591 (2.96), 7.596 |
| | (2.80), 7.616 (0.71), 7.753 (1.64), 7.771 (1.30), 7.824 (2.73), 8.118 (3.98), 8.682 (1.43), |
| | 8.701 (1.43), 8.926 (4.45). |
| 318 | 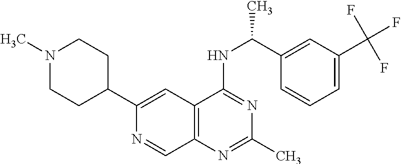 [SMILES: Cc1nc(N[C@H](C)c2cccc(C(F)(F)F)c2)c2cc(C3CCN(C)CC3)ncc2n1] |
| | 2-methyl-6-(1-methylpiperidin-4-yl)-N-{(1R)-1-[3- |
| | (trifluoromethyl)phenyl]ethyl}pyrido[3,4-d]pyrimidin-4-amine |
| | ¹H-NMR (400 MHz, DMSO-d6) δ [ppm]: 1.618 (5.94), 1.635 (5.95), 1.845 (1.01), 1.876 |
| | (1.43), 1.926 (2.05), 1.952 (0.95), 2.128 (1.12), 2.158 (1.90), 2.182 (0.94), 2.287 (12.52), |
| | 2.403 (16.00), 2.729 (0.56), 2.748 (0.54), 2.758 (0.98), 2.768 (0.58), 2.787 (0.45), 2.974 |
| | (1.83), 3.003 (1.71), 5.617 (0.85), 5.635 (1.30), 5.653 (0.86), 7.545 (0.49), 7.565 (1.74), |
| | 7.583 (3.44), 7.606 (0.67), 7.748 (1.56), 7.766 (1.29), 7.817 (2.83), 8.126 (4.01), 8.311 |
| | (4.01), 8.711 (1.27), 8.730 (1.29), 8.916 (5.10). |

### EXAMPLE 319

### methyl 2-methyl-4-({(1R)-1-[3-(trifluoromethyl)phenyl]ethyl}amino)pyrido[3,4-d]pyrimidine-6-carboxylate

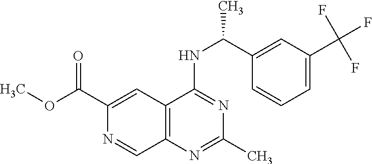 [SMILES: COC(=O)c1cc2c(N[C@H](C)c3cccc(C(F)(F)F)c3)nc(C)nc2cn1]

To a solution of Example 308 (3.00 g, 8.18 mmol), triethylamine (2.3 ml, 16 mmol) in MeOH (60 ml) was added [1,1′-bis(diphenylphosphino)ferrocene]dichloropalladium(ii) (598 mg, 818 μmol) at RT. The reaction mixture was stirred at 80° C. for 18 hours under carbon monoxide atmosphere (50 psi). The reaction mixture was filtered and the filtrate was purified by silica gel column chromatography (petroleum ether: EtOAc) to give the title compound (820 mg, 24%).

### EXAMPLE 320

### 2-methyl-4-({(1R)-1-[3-(trifluoromethyl)phenyl]ethyl}amino)pyrido[3,4-d]pyrimidine-6-carboxamide

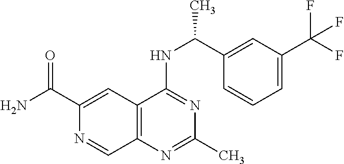 [SMILES: Cc1nc(N[C@H](C)c2cccc(C(F)(F)F)c2)c2cc(C(N)=O)ncc2n1]

Ammonia gas was bubling to ethanol to give a colorless solution at -65° C. To the solution was added Example 319 (100 mg, 251 μmol) at RT. The reaction mixture was stirred in a 30 ml sealed tube at 45° C. for 16 hours The reaction mixture was concentrated to give a residue. The residue was purified by preparative HPLC [Instrument:ACSWH-GX-C; ColumnPhenomenex luna C18 150*25 mm*10 μm; eluent A: water (0.225% formic acid in water), eluent B: acetonitrile; gradient: 0-10 min 25-55% B; flow 25 ml/min; temperature: RT; Detector: UV 220/254 nm.] to give the title compound (55 mg, 58%).

¹H-NMR (400 MHz, DMSO-d6) δ [ppm]: 1.617 (6.33), 1.634 (6.55), 2.324 (0.62), 2.452 (16.00), 2.666 (0.48), 5.629 (1.09), 5.647 (1.63), 5.665 (1.14), 7.545 (0.77), 7.563 (2.11), 7.587 (3.72), 7.731 (2.32), 7.764 (2.24), 7.782 (1.94), 7.852 (3.38), 8.211 (2.19), 8.379 (0.55), 8.952 (4.68), 9.055 (4.64), 9.213 (1.76), 9.231 (1.82).

### EXAMPLE 321

### N,2-dimethyl-4-({(1R)-1-[3-(trifluoromethyl)phenyl]ethyl}amino)pyrido[3,4-d]pyrimidine-6-carboxamide

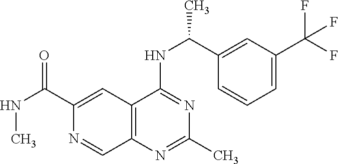 [SMILES: CNC(=O)c1cc2c(N[C@H](C)c3cccc(C(F)(F)F)c3)nc(C)nc2cn1]

To a solution of methylamine in ethanol (2 M) was added Example 319 (120 mg, 307 μmol) at room temperature. The reaction mixture was heated in a sealed tube at 40° C. for 16 hours. The reaction mixture was concentrated to give a residue. The residue was purified by preparative HPLC [Instrument:ACSWH-GX-C; Column: Phenomenex Luna C18 150*25 mm*10 μm; eluent A: water (0.225% formic acid in water), eluent B: acetonitrile; gradient: 0-10 min 25-55% B; flow 25 ml/min; temperature: RT; Detector: UV 220/254 nm.] to give the title compound (32 mg, 26%).

¹H-NMR (400 MHz, DMSO-d6) δ [ppm]: 1.624 (6.20), 1.642 (6.35), 2.452 (16.00), 2.864 (7.76), 2.876 (8.04), 5.633 (0.99), 5.651 (1.50), 5.669 (1.01), 7.547 (0.60), 7.566 (1.94), 7.585 (3.48), 7.589 (3.39), 7.609 (0.81), 7.768 (1.81), 7.785 (1.49), 7.856 (3.12), 8.407 (0.54), 8.858 (1.47), 8.870 (1.49), 8.956 (5.09), 9.023 (4.83), 9.241 (1.66), 9.260 (1.65).

### EXAMPLE 322

### 1-[4-({(1R)-1-[3-(difluoromethyl)-2-methylphenyl]ethyl}amino)-2-methylpyrido[3,4-d]pyrimidin-6-yl]piperidine-4-carbonitrile

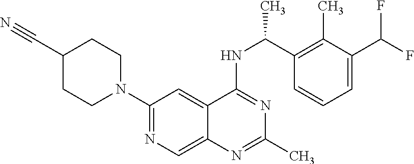 [SMILES: Cc1nc(N[C@H](C)c2cccc(C(F)F)c2C)c2cc(N3CCC(C#N)CC3)ncc2n1]

Using the method described for Example 25: Intermediate 16 (50 mg, 186 μmol) was treated with (1R)-1-[3-(difluoromethyl)-2-fluorophenyl]ethan-1-amine hydrochloride (49.4 mg, 223 μmol) and gave the titled compound (53 mg, 62%) after preparative HPLC purification (basic method).

¹H-NMR (400 MHz, DMSO-d6) δ [ppm]: 1.544 (4.76), 1.561 (4.83), 1.802 (0.71), 1.808 (0.73), 1.831 (0.85), 1.854 (0.41), 2.004 (0.84), 2.318 (16.00), 2.518 (1.48), 2.523 (1.21), 2.534 (7.42), 3.121 (0.45), 3.131 (0.63), 3.142 (0.85), 3.153 (0.62), 3.164 (0.41), 3.385 (0.68), 3.411 (1.02), 3.438 (0.72), 3.873 (0.86), 3.906 (0.76), 5.715 (0.69), 5.732 (1.05), 5.750 (0.68), 7.078 (0.91), 7.216 (1.93), 7.282 (0.65), 7.300 (1.63), 7.320 (1.13), 7.353 (0.79), 7.382 (1.64), 7.400 (1.05), 7.462 (2.93), 7.623 (1.26), 7.641 (1.12), 8.464 (1.13), 8.483 (1.09), 8.645 (4.51).

### EXAMPLE 323

### N-{(1R)-1-[3-(difluoromethyl)-2-fluorophenyl]ethyl}-6-[(2S)-2,4-dimethylpiperazin-1-yl]-2-methylpyrido[3,4-d]pyrimidin-4-amine

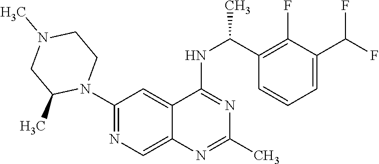 [SMILES: Cc1nc(N[C@H](C)c2cccc(C(F)F)c2F)c2cc(N3CCN(C)C[C@@H]3C)ncc2n1]

Using the method described for Example 25: Intermediate 17 (35.0 mg, 128 μmol) was treated with (1R)-1-[3-(difluoromethyl)-2-fluorophenyl]ethan-1-amine hydrochloride (34.7 mg, 154 μmol) and gave the titled compound (51 mg, 86%) after preparative HPLC purification (basic method).

¹H-NMR (400 MHz, DMSO-d6) δ [ppm]: 0.859 (0.88), 0.967 (2.64), 1.109 (2.22), 1.132 (5.97), 1.144 (2.86), 1.149 (6.06), 1.208 (0.45), 1.224 (0.58), 1.603 (5.21), 1.621 (5.18), 1.989 (0.45), 2.010 (0.70), 2.017 (0.72), 2.038 (0.47), 2.164 (0.75), 2.173 (0.87), 2.192 (0.92), 2.201 (0.87), 2.230 (10.75), 2.298 (16.00), 2.318 (0.49), 2.323 (0.92), 2.327 (1.22), 2.331 (0.87), 2.336 (0.40), 2.518 (5.31), 2.523 (3.58), 2.660 (0.41), 2.665 (0.87), 2.669 (1.20), 2.673 (0.83), 2.747 (1.05), 2.774 (0.96), 2.903 (0.73), 2.931 (0.68), 3.070 (0.49), 3.078 (0.70), 3.101 (0.85), 3.109 (0.77), 3.132 (0.49), 3.164 (0.51), 3.906 (0.70), 3.937 (0.64), 4.684 (0.62), 5.757 (0.77), 5.775 (1.19), 5.793 (0.77), 7.103 (1.19), 7.240 (2.48), 7.278 (0.87), 7.298 (1.92), 7.317 (1.13), 7.344 (3.07), 7.375 (1.05), 7.487 (0.66), 7.504 (1.13), 7.522 (0.55), 7.635 (0.62), 7.653 (1.11), 7.672 (0.55), 8.411 (1.24), 8.430 (1.20), 8.663 (4.82).

### EXAMPLE 324

### {1-[4-({(1R)-1-[3-(difluoromethyl)-2-fluorophenyl]ethyl}amino)-2-methylpyrido[3,4-d]pyrimidin-6-yl]-4-methylpiperazin-2-yl}methanol (mixture of stereoisomers)

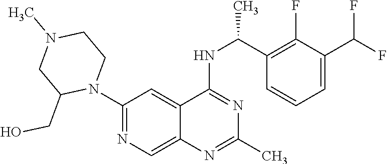 [SMILES: Cc1nc(N[C@H](C)c2cccc(C(F)F)c2F)c2cc(N3CCN(C)CC3CO)ncc2n1]

Using the method described for Example 25: Intermediate 18 (33.0 mg, 145 μmol) was treated with (1R)-1-[3-(difluoromethyl)-2-fluorophenyl]ethan-1-amine hydrochloride (34.7 mg, 154 μmol) and gave the titled compound (32 mg, 55%) after preparative HPLC purification (basic method).

¹H-NMR (400 MHz, DMSO-d6) δ [ppm]: 0.967 (0.55), 1.109 (1.44), 1.598 (4.22), 1.605 (4.42), 1.616 (4.36), 1.623 (4.10), 1.974 (0.46), 2.004 (1.93), 2.014 (1.28), 2.023 (1.17), 2.032 (1.55), 2.233 (14.08), 2.290 (11.34), 2.296 (11.41), 2.518 (16.00), 2.523 (10.44), 2.673 (2.26), 2.877 (0.98), 2.903 (0.91), 3.072 (0.53), 3.109 (2.04), 3.136 (1.60), 3.779 (0.79), 3.919 (0.51), 3.950 (0.54), 3.998 (0.45), 4.550 (0.76), 4.741 (0.81), 4.753 (0.83), 5.747 (0.66), 5.755 (0.74), 5.765 (1.03), 5.773 (1.04), 5.783 (0.71), 5.791 (0.65), 7.103 (1.22), 7.239 (2.48), 7.272 (0.69), 7.281 (0.72), 7.291 (1.48), 7.300 (1.51), 7.310 (0.92), 7.319 (0.87), 7.341 (2.18), 7.361 (2.16), 7.374 (1.22), 7.502 (1.31), 7.635 (0.80), 7.653 (1.42), 7.671 (0.72), 8.408 (0.96), 8.420 (1.19), 8.437 (0.92), 8.633 (3.50), 8.640 (3.50).

### EXAMPLE 325

### N-{(1R)-1-[3-(difluoromethyl)-2-fluorophenyl]ethyl}-2-methyl-6-[2-(trifluoromethyl)-5,6-dihydroimidazo[1,2-a]pyrazin-7(8H)-yl]pyrido[3,4-d]pyrimidin-4-amine

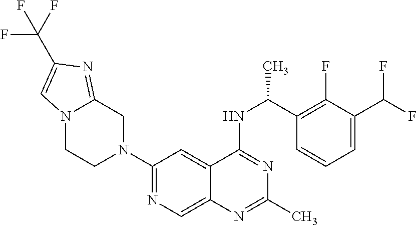 [SMILES: Cc1nc(N[C@H](C)c2cccc(C(F)F)c2F)c2cc(N3CCn4cc(C(F)(F)F)nc4C3)ncc2n1]

Using the method described for Example 25: Intermediate 19 (30 mg, 86 μmol) was treated with (1R)-1-[3-(difluoromethyl)-2-fluorophenyl]ethan-1-amine hydrochloride (23 mg, 103 μmol) and gave the titled compound (15 mg, 33%) after preparative HPLC purification (basic method).

¹H-NMR (400 MHz, DMSO-d6) δ [ppm]: 1.107 (3.17), 1.225 (0.55), 1.348 (0.42), 1.632 (5.40), 1.650 (5.34), 2.325 (16.00), 2.518 (6.33), 2.523 (4.14), 2.660 (0.42), 2.665 (0.91), 2.669 (1.29), 2.673 (0.93), 2.678 (0.42), 4.205 (2.79), 4.215 (3.02), 4.224 (2.01), 4.825 (3.65), 4.830 (3.69), 5.760 (0.82), 5.778 (1.25), 5.796 (0.80), 7.107 (1.20), 7.243 (2.51), 7.285 (0.91), 7.303 (2.01), 7.323 (1.14), 7.379 (1.06), 7.495 (0.70), 7.512 (1.18), 7.530 (0.57), 7.623 (3.25), 7.648 (0.68), 7.666 (1.18), 7.684 (0.59), 7.836 (3.00), 7.839 (3.15), 8.490 (1.33), 8.509 (1.29), 8.741 (4.92).

### EXAMPLE 326

### N-{(1R)-1-[3-(difluoromethyl)-2-fluorophenyl]ethyl}-2-methyl-6-[2-(trifluoromethyl)-5,6-dihydro[1,2,4]triazolo[1,5-a]pyrazin-7(8H)-yl]pyrido[3,4-d]pyrimidin-4-amine

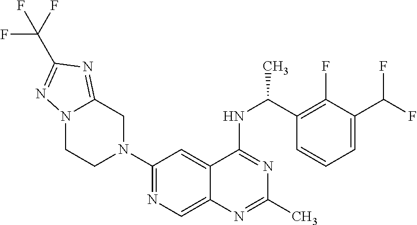 [SMILES: Cc1nc(N[C@H](C)c2cccc(C(F)F)c2F)c2cc(N3CCn4nc(C(F)(F)F)nc4C3)ncc2n1]

Using the method described for Example 25: Intermediate 20 (30 mg, 85 μmol) was treated with (1R)-1-[3-(difluoromethyl)-2-fluorophenyl]ethan-1-amine hydrochloride (23 mg, 102 μmol) and gave the titled compound (15 mg, 31%) after preparative HPLC purification (basic method).

¹H-NMR (400 MHz, DMSO-d6) δ [ppm]: 1.636 (5.12), 1.653 (5.03), 2.330 (16.00), 2.518 (7.79), 2.523 (4.89), 2.665 (1.21), 2.669 (1.66), 2.673 (1.18), 4.302 (1.12), 4.314 (2.28), 4.328 (1.69), 4.441 (1.49), 4.455 (2.14), 4.983 (4.47), 5.762 (0.73), 5.779 (1.18), 5.797 (0.76), 7.108 (1.12), 7.243 (2.39), 7.286 (0.87), 7.305 (1.91), 7.324 (1.10), 7.379 (1.01), 7.498 (0.67), 7.515 (1.12), 7.532 (0.56), 7.649 (0.65), 7.669 (1.29), 7.679 (3.32), 8.488 (1.27), 8.506 (1.21), 8.757 (4.72).

### EXAMPLE 327

### 6-(cyclobutyloxy)-N-{(1R)-1-[3-(difluoromethyl)-2-fluorophenyl]ethyl}-2-methylpyrido[3,4-d]pyrimidin-4-amine

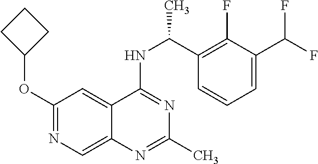 [SMILES: Cc1nc(N[C@H](C)c2cccc(C(F)F)c2F)c2cc(OC3CCC3)ncc2n1]

To sodium hydride (60% dispersion on mineral oil, 28.5 mg, 714 μmol) under Argon was added a solution of cyclobutanol (51.5 mg, 714 μmol) in NMP (2 ml) and stirred for 5 min at RT. Then Example 2 (50 mg, 143 μmol) was added and the reaction heated using a microwave at 180° C. for 20 min. The reaction mixture was diluted with water and extracted with EtOAc. The combined organics were washed with sat. NaCl, filtered through a hydrophobic filter and concentrated. The title compound (6.7 mg, 12%) was isolated after preparative HPLC purification along with the ring-opened side-product (see Example 328, 1.5 mg, 3%).

¹H-NMR (400 MHz, DMSO-d6) δ [ppm]: 1.107 (0.46), 1.228 (0.71), 1.394 (0.41), 1.418 (0.75), 1.443 (0.87), 1.463 (0.58), 1.495 (5.88), 1.512 (5.90), 1.695 (0.54), 1.705 (0.75), 1.721 (0.61), 2.298 (0.59), 2.318 (1.78), 2.323 (1.71), 2.327 (1.61), 2.340 (2.65), 2.361 (1.63), 2.364 (1.64), 2.386 (0.99), 2.412 (16.00), 2.518 (3.94), 2.523 (2.71), 2.539 (0.75), 2.665 (0.48), 2.669 (0.66), 2.673 (0.48), 4.715 (0.89), 4.736 (1.24), 4.754 (0.86), 5.729 (0.83), 5.746 (1.29), 5.764 (0.84), 6.962 (1.03), 7.100 (2.00), 7.205 (1.11), 7.224 (2.48), 7.238 (1.12), 7.243 (1.55), 7.437 (1.61), 7.456 (1.34), 7.617 (1.47), 7.635 (1.35), 8.218 (2.88), 8.744 (4.28), 8.767 (1.39), 8.785 (1.35).

### EXAMPLE 328

### 6-butoxy-N-{(1R)-1-[3-(difluoromethyl)-2-fluorophenyl]ethyl}-2-methylpyrido[3,4-d]pyrimidin-4-amine

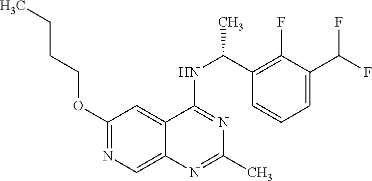 [SMILES: CCCCOc1cc2c(N[C@H](C)c3cccc(C(F)F)c3F)nc(C)nc2cn1]

Isolated as a side-product (see Example 327).

¹H-NMR (400 MHz, DMSO-d6) δ [ppm]: 0.832 (0.50), 0.850 (0.77), 0.947 (4.12), 0.965 (9.91), 0.984 (4.82), 1.229 (2.06), 1.347 (0.53), 1.496 (1.46), 1.511 (6.55), 1.528 (6.09), 1.553 (1.00), 1.807 (0.57), 1.824 (1.56), 1.842 (2.13), 1.859 (1.36), 1.878 (0.50), 2.322 (1.03), 2.326 (1.20), 2.331 (0.86), 2.382 (16.00), 2.412 (2.06), 2.522 (4.32), 2.664 (0.77), 2.669 (1.03), 2.673 (0.77), 2.692 (0.60), 2.722 (0.53), 2.856 (0.43), 3.300 (0.47), 3.898 (0.47), 3.913 (1.06), 3.936 (1.13), 3.952 (0.47), 4.425 (0.50), 4.441 (1.16), 4.464 (1.10), 4.481 (0.47), 5.814 (0.83), 5.831 (1.26), 5.850 (0.83), 6.999 (1.03), 7.136 (1.96), 7.236 (1.23), 7.256 (2.40), 7.275 (2.16), 7.458 (1.73), 7.477 (1.33), 7.664 (1.46), 7.683 (1.36), 8.201 (2.86), 8.218 (0.43), 8.738 (4.39), 8.776 (1.33), 8.794 (1.26).

TABLE 15
Examples 329-337
Following the method described here for Example 327, the following Examples in Table 15 were
prepared either as their mono- or di-substituted analogs.

| | Structure |
|---|---|
| | IUPAC-Name |
| Example | ¹H-NMR |
| 329 | 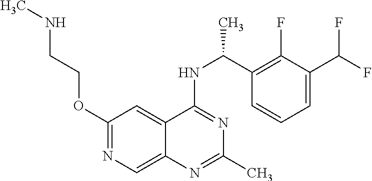 [SMILES: CNCCOc1cc2c(N[C@H](C)c3cccc(C(F)F)c3F)nc(C)nc2cn1] |
| | N-{(1R)-1-[3-(difluoromethyl)-2-fluorophenyl]ethyl}-2-methyl-6-[2- |
| | (methylamino)ethoxy]pyrido[3,4-d]pyrimidin-4-amine |
| | ¹H-NMR (400 MHz, DMSO-d6) δ [ppm]: 1.230 (1.50), 1.460 (4.62), 1.477 (4.75), 1.517 |
| | (1.04), 1.534 (0.98), 1.564 (0.52), 2.247 (0.85), 2.279 (0.46), 2.323 (16.00), 2.346 (13.27), |
| | 2.373 (3.25), 2.380 (3.32), 2.392 (2.41), 2.523 (6.05), 2.539 (1.69), 2.665 (1.17), 2.669 |
| | (1.63), 2.673 (1.17), 2.869 (0.59), 2.903 (1.17), 2.963 (0.98), 2.977 (0.78), 2.996 (0.52), |
| | 3.012 (0.78), 3.131 (2.86), 3.300 (0.46), 3.956 (0.46), 3.970 (0.91), 3.983 (0.85), 3.994 |
| | (0.98), 4.616 (0.72), 4.639 (0.72), 5.727 (0.78), 5.744 (1.17), 5.762 (0.78), 7.039 (0.91), |
| | 7.177 (1.76), 7.188 (1.30), 7.207 (2.41), 7.226 (1.50), 7.315 (0.85), 7.417 (1.63), 7.436 |
| | (1.56), 7.455 (0.52), 7.561 (1.50), 7.579 (1.43), 8.087 (2.86), 8.203 (0.46), 8.655 (4.81), |
| | 8.688 (1.56), 8.706 (1.50), 8.735 (0.98). |
| 330 | 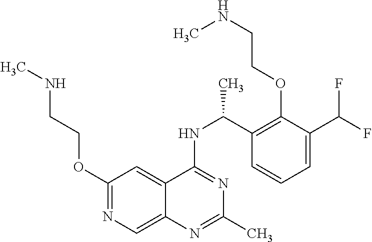 [SMILES: CNCCOc1cc2c(N[C@H](C)c3cccc(C(F)F)c3OCCNC)nc(C)nc2cn1] |
| | N-[(1R)-1-{3-(difluoromethyl)-2-[2-(methylamino)ethoxy]phenyl}ethyl]-2-methyl-6-[2- |
| | (methylamino)ethoxy]pyrido[3,4-d]pyrimidin-4-amine |
| | ¹H-NMR (400 MHz, DMSO-d6) δ [ppm]: 1.107 (16.00), 1.228 (0.86), 1.499 (3.71), 1.517 |
| | (4.05), 1.536 (0.87), 1.552 (0.53), 2.302 (0.68), 2.330 (12.24), 2.341 (3.07), 2.356 (14.03), |
| | 2.378 (14.80), 2.518 (3.09), 2.523 (1.94), 2.540 (8.32), 2.665 (0.58), 2.669 (0.80), 2.674 |
| | (0.58), 2.858 (1.64), 2.872 (3.29), 2.886 (1.95), 2.912 (1.02), 2.924 (0.65), 2.934 (0.58), |
| | 2.945 (0.78), 2.950 (0.71), 2.961 (0.68), 3.021 (0.61), 3.968 (0.56), 3.976 (0.61), 3.986 |
| | (0.56), 3.991 (0.59), 4.192 (0.51), 4.343 (1.72), 4.358 (3.28), 4.372 (1.61), 4.579 (0.57), |
| | 4.592 (0.61), 4.602 (0.53), 5.829 (0.59), 5.847 (0.87), 5.865 (0.60), 7.131 (0.70), 7.228 |
| | (0.84), 7.247 (1.70), 7.258 (0.40), 7.268 (1.96), 7.406 (0.65), 7.425 (0.42), 7.445 (1.28), |
| | 7.464 (0.94), 7.653 (1.01), 7.671 (0.92), 7.810 (2.92), 8.562 (0.96), 8.581 (0.93), 8.675 |
| | (0.49), 8.690 (3.55), 8.734 (0.42). |
| 331 | 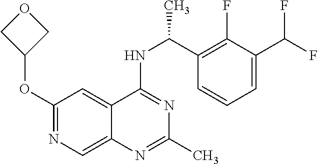 [SMILES: Cc1nc(N[C@H](C)c2cccc(C(F)F)c2F)c2cc(OC3COC3)ncc2n1] |
| | N-{(1R)-1-[3-(difluoromethyl)-2-fluorophenyl]ethyl}-2-methyl-6-[(oxetan-3- |
| | yl)oxy]pyrido[3,4-d]pyrimidin-4-amine |
| | ¹H-NMR (400 MHz, DMSO-d6) δ [ppm]: 1.228 (1.19), 1.504 (5.40), 1.521 (5.34), 2.451 |
| | (16.00), 2.518 (2.30), 2.522 (1.42), 2.539 (5.40), 4.824 (0.82), 4.841 (2.19), 4.858 (1.46), |
| | 4.886 (1.27), 4.907 (4.27), 4.923 (4.25), 5.317 (1.15), 5.332 (1.53), 5.348 (1.00), 5.432 |
| | (0.80), 5.450 (1.23), 5.468 (0.79), 6.976 (1.00), 7.113 (1.83), 7.245 (1.21), 7.250 (1.06), |
| | 7.264 (2.24), 7.284 (1.29), 7.440 (1.49), 7.459 (1.23), 7.644 (1.35), 7.663 (1.21), 8.196 |
| | (2.53), 8.198 (2.61), 8.749 (4.28), 8.791 (1.29), 8.808 (1.25). |
| 332 | 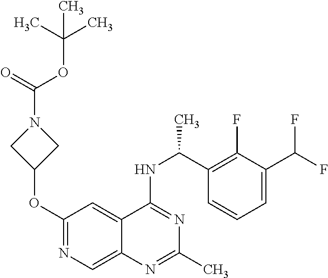 [SMILES: Cc1nc(N[C@H](C)c2cccc(C(F)F)c2F)c2cc(OC3CN(C(=O)OC(C)(C)C)C3)ncc2n1] |
| | tert-butyl 3-{[4-({(1R)-1-[3-(difluoromethyl)-2-fluorophenyl]ethyl}amino)-2- |
| | methylpyrido[3,4-d]pyrimidin-6-yl]oxy}azetidine-1-carboxylate |
| | ¹H-NMR (400 MHz, DMSO-d6) δ [ppm]: 1.394 (16.00), 1.588 (1.64), 1.606 (1.66), 2.349 |
| | (5.23), 2.518 (1.26), 2.522 (0.78), 5.758 (0.43), 7.233 (0.81), 7.289 (0.62), 7.850 (1.22), |
| | 8.626 (0.40), 8.679 (1.49). |
| 333 | 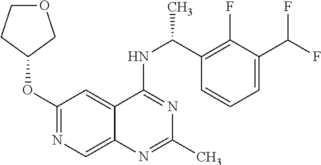 [SMILES: Cc1nc(N[C@H](C)c2cccc(C(F)F)c2F)c2cc(O[C@@H]3CCOC3)ncc2n1] |
| | N-{(1R)-1-[3-(difluoromethyl)-2-fluorophenyl]ethyl}-2-methyl-6-{[(3R)-oxolan-3- |
| | yl]oxy}pyrido[3,4-d]pyrimidin-4-amine |
| | ¹H-NMR (400 MHz, DMSO-d6) δ [ppm]: 1.154 (3.47), 1.171 (6.41), 1.189 (3.08), 1.230 |
| | (0.48), 1.499 (5.28), 1.516 (5.32), 1.987 (12.17), 2.224 (0.41), 2.237 (0.68), 2.250 (0.43), |
| | 2.259 (0.77), 2.272 (0.61), 2.346 (0.81), 2.357 (16.00), 2.393 (0.53), 2.404 (0.48), 2.408 |
| | (0.51), 2.411 (0.52), 2.423 (0.53), 2.518 (2.43), 2.522 (1.53), 3.642 (1.18), 3.651 (1.30), |
| | 3.669 (1.53), 3.678 (1.43), 3.828 (0.64), 3.838 (0.68), 3.849 (1.02), 3.859 (1.12), 3.871 |
| | (0.71), 3.881 (0.66), 3.906 (1.63), 3.933 (1.33), 3.999 (0.92), 4.017 (2.74), 4.034 (2.70), |
| | 4.052 (0.88), 4.062 (0.64), 4.083 (1.52), 4.101 (1.46), 4.121 (0.51), 5.337 (0.73), 5.349 |
| | (1.05), 5.359 (0.73), 5.749 (0.77), 5.767 (1.16), 5.784 (0.75), 6.989 (0.93), 7.126 (1.80), |
| | 7.250 (1.09), 7.269 (2.48), 7.289 (1.36), 7.492 (1.43), 7.511 (1.19), 7.647 (1.30), 7.666 |
| | (1.19), 8.226 (2.52), 8.228 (2.53), 8.747 (4.43), 8.770 (1.29), 8.788 (1.25). |
| 334 | 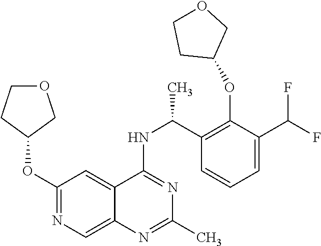 [SMILES: Cc1nc(N[C@H](C)c2cccc(C(F)F)c2O[C@@H]2CCOC2)c2cc(O[C@@H]3CCOC3)ncc2n1] |
| | N-{(1R)-1-[3-(difluoromethyl)-2-{[(3R)-oxolan-3-yl]oxy}phenyl]ethyl}-2-methyl-6-{[(3R)- |
| | oxolan-3-yl]oxy}pyrido[3,4-d]pyrimidin-4-amine |
| | ¹H-NMR (400 MHz, DMSO-d6) δ [ppm]: 1.232 (1.19), 1.474 (4.00), 1.491 (4.03), 1.859 |
| | (0.75), 1.880 (2.03), 1.899 (2.47), 1.914 (1.69), 1.918 (2.28), 1.936 (0.91), 2.029 (0.47), |
| | 2.047 (0.50), 2.064 (0.56), 2.155 (2.34), 2.175 (3.34), 2.195 (1.81), 2.227 (0.50), 2.240 |
| | (0.91), 2.260 (1.34), 2.278 (1.00), 2.309 (12.16), 2.323 (1.53), 2.327 (1.91), 2.331 (1.66), |
| | 2.418 (0.47), 2.438 (0.63), 2.518 (9.19), 2.523 (6.37), 2.665 (1.31), 2.669 (1.75), 2.673 |
| | (1.28), 2.692 (16.00), 3.283 (3.94), 3.301 (5.03), 3.319 (5.59), 3.633 (1.16), 3.642 (1.25), |
| | 3.660 (1.25), 3.669 (1.16), 3.796 (0.47), 3.816 (2.03), 3.827 (1.56), 3.837 (2.22), 3.849 |
| | (1.97), 3.860 (1.03), 3.870 (1.53), 3.887 (1.16), 3.903 (1.41), 3.930 (1.12), 3.953 (1.25), |
| | 3.964 (1.37), 3.978 (1.12), 3.989 (0.91), 4.063 (0.50), 4.083 (1.22), 4.103 (1.16), 5.391 |
| | (0.91), 5.582 (0.97), 5.586 (0.94), 5.597 (0.59), 5.740 (0.63), 5.758 (0.94), 5.775 (0.63), |
| | 6.981 (0.69), 7.118 (1.37), 7.239 (0.91), 7.258 (2.12), 7.277 (1.06), 7.479 (1.19), 7.497 |
| | (1.00), 7.636 (1.03), 7.655 (0.97), 7.875 (3.19), 8.564 (1.06), 8.582 (1.03), 8.716 (3.72). |
| 335 | 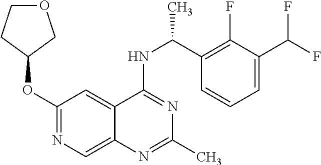 [SMILES: Cc1nc(N[C@H](C)c2cccc(C(F)F)c2F)c2cc(O[C@H]3CCOC3)ncc2n1] |
| | N-{(1R)-1-[3-(difluoromethyl)-2-fluorophenyl]ethyl}-2-methyl-6-{[(3S)-oxolan-3- |
| | yl]oxy}pyrido[3,4-d]pyrimidin-4-amine |
| | ¹H-NMR (400 MHz, DMSO-d6) δ [ppm]: 1.154 (4.28), 1.171 (8.10), 1.190 (4.03), 1.498 |
| | (4.00), 1.516 (4.00), 1.987 (16.00), 2.142 (0.57), 2.155 (0.56), 2.163 (0.82), 2.176 (0.91), |
| | 2.197 (0.70), 2.210 (0.51), 2.226 (0.41), 2.332 (0.53), 2.382 (11.85), 2.518 (1.80), 2.523 |
| | (1.16), 2.673 (0.42), 2.692 (1.59), 3.283 (0.42), 3.301 (0.60), 3.318 (1.19), 3.781 (0.94), |
| | 3.792 (1.06), 3.807 (1.24), 3.817 (1.24), 3.825 (1.08), 3.836 (1.11), 3.846 (0.59), 3.857 |
| | (0.54), 3.979 (0.52), 3.999 (2.11), 4.017 (4.43), 4.034 (3.60), 4.052 (1.13), 4.278 (1.18), |
| | 4.304 (1.06), 5.140 (0.48), 5.144 (0.55), 5.153 (0.77), 5.163 (0.47), 5.712 (0.56), 5.730 |
| | (0.87), 5.748 (0.56), 7.035 (0.68), 7.171 (1.20), 7.236 (0.80), 7.255 (1.70), 7.275 (0.97), |
| | 7.308 (0.59), 7.491 (1.05), 7.509 (0.88), 7.610 (0.93), 7.630 (0.84), 8.217 (1.84), 8.220 |
| | (1.85), 8.734 (0.97), 8.747 (3.63). |
| 336 | 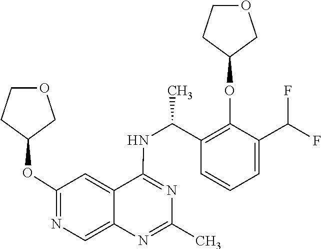 [SMILES: Cc1nc(N[C@H](C)c2cccc(C(F)F)c2O[C@H]2CCOC2)c2cc(O[C@H]3CCOC3)ncc2n1] |
| | N-{(1R)-1-[3-(difluoromethyl)-2-{[(3S)-oxolan-3-yl]oxy}phenyl]ethyl]-2-methyl-6-{[(3S)- |
| | oxolan-3-yl]oxy}pyrido[3,4-d]pyrimidin-4-amine |
| | ¹H-NMR (400 MHz, DMSO-d6) δ [ppm]: 1.107 (16.00), 1.473 (2.17), 1.490 (2.25), 1.879 |
| | (0.89), 1.898 (1.12), 1.914 (0.75), 1.917 (1.03), 1.934 (0.43), 2.153 (1.34), 2.166 (0.53), |
| | 2.174 (1.71), 2.195 (1.06), 2.225 (0.43), 2.247 (0.47), 2.262 (0.48), 2.332 (6.54), 2.692 |
| | (7.12), 3.282 (1.56), 3.301 (2.12), 3.318 (1.87), 3.797 (1.13), 3.803 (0.89), 3.807 (1.03), |
| | 3.817 (0.93), 3.824 (1.82), 3.834 (1.28), 3.849 (0.66), 3.853 (0.63), 3.874 (0.68), 3.891 |
| | (0.57), 3.945 (0.64), 3.956 (0.74), 3.970 (0.60), 3.977 (0.43), 3.982 (0.57), 3.998 (0.64), |
| | 4.015 (0.61), 4.190 (1.62), 4.287 (0.70), 4.312 (0.63), 5.187 (0.50), 5.583 (0.53), 5.588 |
| | (0.53), 5.712 (0.51), 7.163 (0.71), 7.221 (0.46), 7.241 (0.98), 7.260 (0.58), 7.476 (0.65), |
| | 7.495 (0.56), 7.597 (0.59), 7.616 (0.54), 7.865 (1.70), 8.522 (0.56), 8.541 (0.56), 8.713 |
| | (2.01). |
| 337 | 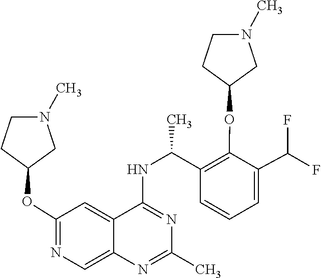 [SMILES: Cc1nc(N[C@H](C)c2cccc(C(F)F)c2O[C@H]2CCN(C)C2)c2cc(O[C@H]3CCN(C)C3)ncc2n1] |
| | N-{(1R)-1-[3-(difluoromethyl)-2-{[(3S)-1-methylpyrrolidin-3-yl]oxy}phenyl]ethyl}-2- |
| | methyl-6-{[(3S)-1-methylpyrrolidin-3-yl]oxy}pyrido[3,4-d]pyrimidin-4-amine |
| | ¹H-NMR (400 MHz, DMSO-d6) δ [ppm]: 1.107 (1.89), 1.485 (2.77), 1.502 (2.82), 2.242 |
| | (0.48), 2.262 (1.03), 2.274 (16.00), 2.291 (0.51), 2.306 (0.55), 2.326 (1.08), 2.338 (8.86), |
| | 2.391 (0.44), 2.406 (0.45), 2.412 (0.54), 2.428 (0.52), 2.518 (1.57), 2.522 (1.01), 2.567 |
| | (0.51), 2.575 (0.54), 2.586 (0.62), 2.594 (0.71), 2.600 (1.12), 2.612 (0.65), 2.627 (0.58), |
| | 2.660 (0.63), 2.664 (0.53), 2.669 (0.63), 2.673 (0.77), 2.828 (0.54), 2.839 (0.55), 2.859 |
| | (0.76), 2.874 (0.68), 2.884 (0.58), 2.900 (0.51), 3.083 (0.56), 3.089 (0.55), 3.110 (0.51), |
| | 3.115 (0.50), 4.983 (0.46), 4.987 (0.45), 5.400 (0.46), 5.404 (0.46), 5.758 (0.44), 5.763 |
| | (0.42), 5.780 (0.63), 5.798 (0.41), 7.066 (0.48), 7.204 (1.35), 7.225 (1.28), 7.244 (0.73), |
| | 7.340 (0.41), 7.454 (0.82), 7.473 (0.69), 7.585 (0.75), 7.604 (0.68), 7.808 (2.23), 8.464 |
| | (0.74), 8.483 (0.71), 8.694 (2.66). |

### EXAMPLE 338

### 6-[(azetidin-3-yl)oxy]-N-{(1R)-1-[3-(difluoromethyl)-2-fluorophenyl]ethyl}-2-methylpyrido[3,4-d]pyrimidin-4-amine hydrochloride

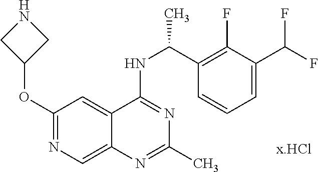 [SMILES: Cc1nc(N[C@H](C)c2cccc(C(F)F)c2F)c2cc(OC3CNC3)ncc2n1]

To a solution of Example 332 (13.4 mg, 26.6 μmol) in dioxane (130 μl) was added a HCl solution in dioxane (4M, 130 μmol) and stirred at RT for 1 h. The reaction was concentrated to give the title compound (13 mg).

¹H-NMR (400 MHz, DMSO-d6) δ [ppm]: 1.107 (16.00), 1.232 (0.51), 1.593 (0.96), 1.669 (2.47), 1.686 (2.49), 1.709 (1.27), 1.727 (1.17), 1.907 (0.50), 2.332 (0.76), 2.423 (6.04), 2.431 (6.05), 2.518 (4.08), 2.523 (2.61), 2.579 (2.11), 2.673 (0.75), 3.384 (0.82), 3.675 (0.45), 4.064 (0.40), 5.248 (0.50), 5.281 (0.40), 5.706 (0.40), 5.792 (0.53), 5.810 (0.53), 7.096 (0.51), 7.103 (0.78), 7.231 (1.04), 7.238 (1.62), 7.290 (0.44), 7.307 (0.96), 7.326 (0.57), 7.357 (0.73), 7.367 (0.58), 7.375 (1.10), 7.512 (0.53), 7.529 (0.88), 7.549 (0.49), 7.573 (0.43), 7.826 (0.44), 8.620 (0.84), 8.879 (0.51), 9.557 (3.01).

### EXAMPLE 339

### tert-butyl {(3-trans)-1-[4-({(1R)-1-[3-(difluoromethyl)-2-fluorophenyl]ethyl}amino)-2-methylpyrido[3,4-d]pyrimidin-6-yl]-4-fluoropyrrolidin-3-yl}carbamate (mixture of stereoisomers)

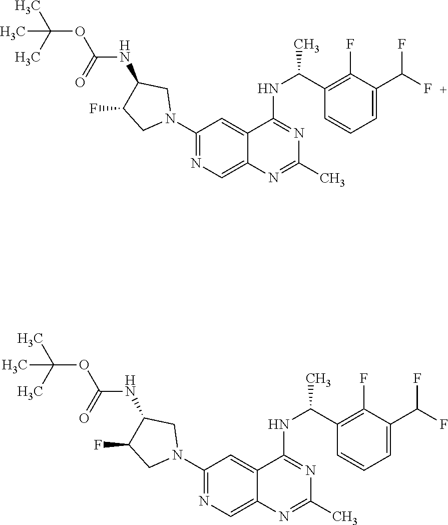 [SMILES: Cc1nc(N[C@H](C)c2cccc(C(F)F)c2F)c2cc(N3C[C@H](NC(=O)OC(C)(C)C)[C@@H](F)C3)ncc2n1 ; Cc1nc(N[C@H](C)c2cccc(C(F)F)c2F)c2cc(N3C[C@@H](F)[C@H](NC(=O)OC(C)(C)C)C3)ncc2n1]

To a solution of Example 2 (50.0 mg, 143 μmol) in DMSO (1.3 ml) was added tert-butyl [rac-(trans)-4-fluoropyrrolidin-3-yl]carbamate (58.3 mg, 285 μmol) and TEA (80 μl, 570 μmol). The reaction was heated at 110° C. for 16 h. Another portion of the amine was added (58.3 mg, 285 μmol) and TEA (80 μl, 570 μmol) were added and heated at 130° C. for 16 h. The reaction was allowed to cool and then purified by preparative HPLC (basic method) to give the titled compound (23 mg, 28%).

¹H-NMR (400 MHz, DMSO-d6) δ [ppm]: 1.107 (2.26), 1.404 (16.00), 1.612 (4.71), 1.630 (4.52), 2.273 (0.50), 2.300 (10.47), 2.327 (0.45), 2.401 (0.69), 2.518 (1.82), 2.522 (1.09), 2.725 (0.51), 3.489 (0.51), 3.501 (0.49), 3.517 (0.61), 3.746 (0.64), 3.762 (0.74), 3.776 (1.11), 3.803 (0.71), 5.155 (0.55), 5.284 (0.55), 5.763 (0.55), 5.780 (0.82), 5.796 (0.53), 7.102 (1.09), 7.148 (2.78), 7.237 (2.25), 7.273 (0.75), 7.293 (1.64), 7.312 (0.97), 7.373 (0.96), 7.454 (0.61), 7.469 (0.61), 7.486 (0.71), 7.503 (1.08), 7.521 (0.54), 7.632 (0.54), 7.650 (0.99), 7.668 (0.55), 8.409 (1.13), 8.427 (1.10), 8.655 (3.98).

### EXAMPLE 340

### 6-[(trans)-3-amino-4-fluoropyrrolidin-1-yl]-N-{(1R)-1-[3-(difluoromethyl)-2-fluorophenyl]ethyl}-2-methylpyrido[3,4-d]pyrimidin-4-amine hydrochloride (mixture of stereoisomers)

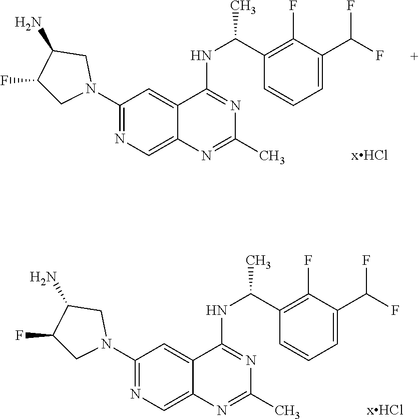 [SMILES: Cc1nc(N[C@H](C)c2cccc(C(F)F)c2F)c2cc(N3C[C@H](N)[C@@H](F)C3)ncc2n1 ; Cc1nc(N[C@H](C)c2cccc(C(F)F)c2F)c2cc(N3C[C@@H](N)[C@H](F)C3)ncc2n1]

Using the method described for Example 338: Example 339 (17.1 mg, 32.0 μmol) gave the titled compound (16.6 mg).

¹H-NMR (400 MHz, DMSO-d6) δ [ppm]: 1.107 (16.00), 1.143 (0.40), 1.223 (1.20), 1.231 (0.61), 1.740 (3.25), 1.757 (3.26), 2.323 (0.47), 2.327 (0.65), 2.332 (0.46), 2.518 (2.97), 2.523 (2.22), 2.537 (7.42), 2.665 (0.46), 2.669 (0.64), 2.673 (0.45), 2.737 (0.40), 3.841 (0.68), 3.919 (0.65), 3.934 (1.02), 3.950 (0.76), 3.965 (0.69), 3.982 (0.41), 4.160 (0.55), 5.510 (0.59), 5.634 (0.61), 5.983 (0.68), 5.992 (0.63), 6.000 (0.48), 7.109 (0.92), 7.244 (1.87), 7.338 (0.87), 7.357 (1.88), 7.378 (1.79), 7.551 (0.73), 7.568 (1.21), 7.585 (0.59), 7.941 (0.73), 8.730 (0.89), 8.790 (0.74), 8.844 (1.61), 8.860 (1.31).

### EXAMPLE 341

### tert-butyl {(cis)-1-[4-({(11R)-1-[3-(difluoromethyl)-2-fluorophenyl]ethyl}amino)-2-methylpyrido[3,4-d]pyrimidin-6-yl]-4-fluoropyrrolidin-3-yl}carbamate (mixture of stereoisomers)

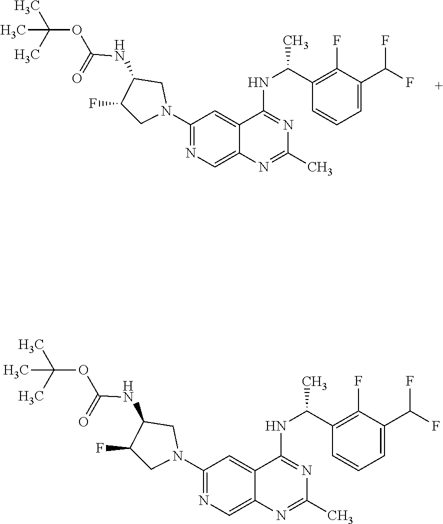 [SMILES: Cc1nc(N[C@H](C)c2cccc(C(F)F)c2F)c2cc(N3C[C@H](F)[C@H](NC(=O)OC(C)(C)C)C3)ncc2n1 ; Cc1nc(N[C@H](C)c2cccc(C(F)F)c2F)c2cc(N3C[C@@H](F)[C@@H](NC(=O)OC(C)(C)C)C3)ncc2n1]

Using the method described for Example 339: Example 2 (17.1 mg, 32.0 μmol) treated with tert-butyl [rac-(cis)-4-fluoropyrrolidin-3-yl]carbamate (58.3 mg, 285 μmol) gave the titled compound (16 mg, 20%) after preparative HPLC purification (basic method).

¹H-NMR (400 MHz, DMSO-d6) δ [ppm]: 1.231 (0.46), 1.433 (16.00), 1.608 (2.37), 1.626 (2.37), 2.294 (4.13), 2.298 (4.49), 2.326 (0.43), 2.518 (1.77), 2.522 (1.09), 2.669 (0.42), 3.314 (0.59), 3.828 (0.65), 3.847 (0.68), 3.870 (0.50), 5.774 (0.60), 7.102 (0.61), 7.121 (1.22), 7.238 (1.26), 7.291 (0.68), 7.374 (0.67), 7.400 (0.42), 7.502 (0.51), 7.647 (0.56), 8.402 (0.48), 8.420 (0.48), 8.648 (2.34).

### EXAMPLE 342

### 6-[(cis)-3-amino-4-fluoropyrrolidin-1-yl]-N-{(1R)-1-[3-(difluoromethyl)-2-fluorophenyl]ethyl}-2-methylpyrido[3,4-d]pyrimidin-4-amine hydrochloride (mixture of stereoisomers)

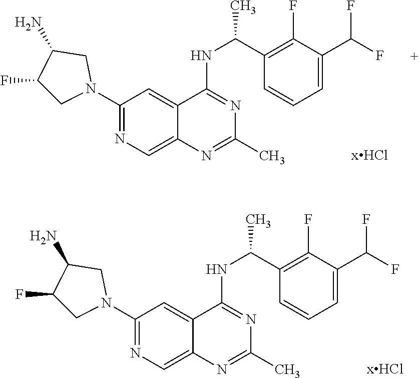 [SMILES: Cc1nc(N[C@H](C)c2cccc(C(F)F)c2F)c2cc(N3C[C@@H](N)[C@@H](F)C3)ncc2n1 ; Cc1nc(N[C@H](C)c2cccc(C(F)F)c2F)c2cc(N3C[C@@H](F)[C@@H](N)C3)ncc2n1]

Using the method described for Example 338: Example 341 (13.3 mg, 24.9 μmol) gave the titled compound (13 mg).

¹H-NMR (400 MHz, DMSO-d6) δ [ppm]: 1.107 (16.00), 1.224 (1.24), 1.232 (0.79), 1.731 (3.65), 1.748 (3.62), 2.323 (0.77), 2.327 (1.07), 2.332 (0.77), 2.518 (7.03), 2.523 (8.51), 2.665 (0.79), 2.669 (1.11), 2.673 (0.77), 3.504 (0.41), 3.526 (0.77), 3.542 (0.81), 3.565 (0.45), 3.899 (0.45), 3.935 (0.69), 3.965 (0.52), 3.974 (0.49), 3.999 (0.41), 4.064 (0.52), 4.088 (0.69), 4.109 (0.58), 5.482 (0.64), 5.620 (0.62), 5.953 (0.47), 5.969 (0.67), 5.985 (0.47), 7.107 (1.01), 7.243 (2.10), 7.336 (0.69), 7.355 (1.52), 7.378 (1.42), 7.552 (0.69), 7.569 (1.18), 7.587 (0.60), 7.891 (0.58), 8.827 (2.34).

Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

In vitro metabolic stability in human liver microsomes. The in vitro metabolic stability of test compounds was determined by incubating them at 1 μM in a suspension of liver microsomes in 100 mM phosphate buffer, pH 7.4 (NaH2PO4×H2O+Na2HPO4×2H2O) and at a protein concentration of 0.5 mg/mL at 37° C. The microsomes were activated by adding a co-factor mix containing 8 mM Glucose-6-phosphate, 4 mM MgCl2, 0.5 mM NADP and 1 IU/ml G-6-P-Dehydrogenase in phosphate buffer, pH 7.4. The metabolic assay was started shortly afterwards by adding the test compound to the incubation at a final volume of 1 mL. Organic solvent in the incubations was limited to 0.01% dimethylsulfoxide (DMSO) and ≤1% acetonitrile. During incubation, the microsomal suspensions were continuously shaken at 580 rpm and aliquots were taken at 2, 8, 16, 30, 45 and 60 min, to which equal volumes of cold methanol were immediately added. Samples were frozen at −20° C. overnight, subsequently centrifuged for 15 minutes at 3000 rpm and the supernatant was analyzed with an Agilent 1200 HPLC-system with LC/MS-MS detection. The half-life of a test compound was determined from the concentration-time plot. From the half-life the intrinsic clearances and the hepatic in vivo blood clearance (CL) and maximal oral bioavailability (Fmax) were calculated using the ‘well stirred’ liver model together with the additional parameters liver blood flow, specific liver weight and microsomal protein content. The following parameter values were used: Liver blood flow 1.32 L/h/kg, specific liver weight 21 g/kg, microsomal protein content 40 mg/g.

In Vitro Metabolic Stability in Rat Hepatocytes.

Hepatocytes from Han/Wistar rats were isolated via a 2-step perfusion method. After perfusion, the liver was carefully removed from the rat: the liver capsule was opened and the hepatocytes were gently shaken out into a Petri dish with ice-cold Williams' medium E (WME). The resulting cell suspension was filtered through sterile gaze in 50 ml falcon tubes and centrifuged at 50×g for 3 min at room temperature. The cell pellet was resuspended in 30 ml WME and centrifuged twice through a Percoll® gradient at 100×g. The hepatocytes were washed again with WME and resuspended in medium containing 5% FCS. Cell viability was determined by trypan blue exclusion. For the metabolic stability assay liver cells were distributed in WME containing 5% FCS to glass vials at a density of 1.0×106 vital cells/ml. The test compound was added to a final concentration of 1 μM. During incubation, the hepatocyte suspensions were continuously shaken at 580 rpm and aliquots were taken at 2, 8, 16, 30, 45 and 90 min, to which equal volumes of cold methanol were immediately added. Samples were frozen at −20° C. overnight, subsequently centrifuged for 15 minutes at 3000 rpm and the supernatant was analyzed with an Agilent 1200 HPLC-system with LC/MS-MS detection. The half-life of a test compound was determined from the concentration-time plot. From the half-life the intrinsic clearances and the hepatic in vivo blood clearance (CL) and maximal oral bioavailability (Fmax) were calculated using the ‘well stirred’ liver model together with the additional parameters liver blood flow, specific liver weight and amount of liver cells in vivo and in vitro. The following parameter values were used: Liver blood flow 4.2 L/h/kg, specific liver weight 32 g/kg, liver cells in vivo 1.1×108 cells/g liver, liver cells in vitro 1.0×106/ml.

Caco-2 Permeability Assay.

Caco-2 cells (purchased from DSMZ Braunschweig, Germany) were seeded at a density of 4.5×10⁴ cells/well on 24-well insert plates, 0.4 μm pore size, and grown for 15 d in DMEM supplemented with 10% FCS, 1% GlutaMAX (100×, Gibco), 100 U/mL penicillin, 100 μg/mL streptomycin (Gibco) and 1% non-essential amino acids (100×). Cells were maintained at 37° C. in a humidified 5% CO₂ atmosphere. Medium was changed every 2-3 d. Before the permeation assay was run, the culture medium was replaced by FCS-free HEPES carbonate transport buffer (pH 7.2) For the assessment of monolayer integrity, the transepithelial electrical resistance was measured. Test compounds were predissolved in DMSO and added either to the apical or basolateral compartment at a final concentration of 2 μM. Before and after incubation for 2 h at 37° C., samples were taken from both compartments and analyzed by LC-MS/MS after precipitation with MeOH. Permeability (Pₐₚₚ) was calculated in the apical to basolateral (A→B) and basolateral to apical (B→A) directions. The apparent permeability was calculated using following equation: Pₐₚₚ=(Vᵣ/P₀)(1/S)(P₂/t), where Vᵣ is the volume of medium in the receiver chamber, P₀ is the measured peak area of the test drug in the donor chamber at t=0, S is the surface area of the monolayer, P₂ is the measured peak area of the test drug in the acceptor chamber after incubation for 2 h, and t is the incubation time. The efflux ratio basolateral (B) to apical (A) was calculated as Pₐₚₚ B−A/Pₐₚₚ A−B. In addition, the compound recovery was calculated. As an assay control, reference compounds were analyzed in parallel.

### 6,7-dimethoxy-N-[(1R)-1-(1-naphthyl)ethyl]quinazolin-4-amine,

which was used to calibrate the assay, was prepared as follows:

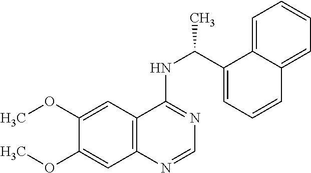 [SMILES: COc1cc2ncnc(N[C@H](C)c3cccc4ccccc34)c2cc1OC]

To 4-chloro-6,7-dimethoxyquinazoline (100 mg, 0.445 mmol, commercially available) in 1.7 mL DMSO was added (1R)-1-(1-naphthyl)ethanamine (76 mg, 0.445 mmol, commercially available) and N-ethyl-N-isopropylpropan-2-amine (202 μl, 1.16 mmol). The reaction was stirred at 100° C. overnight, cooled to ambient temperature and filtered. After removal of the solvent under reduced pressure the crude product was purified via HPLC chromatography to yield the title compound (118 mg, 73%). ¹H-NMR (400 MHz, DMSO-d6), d [ppm]=1.72 (3H), 3.90 (6H), 6.32-6.41 (1H), 7.09 (1H), 7.46-7.58 (3H), 7.64-7.69 (1H), 7.78 (2H), 7.92-7.97 (1H), 8.18-8.24 (2H), 8.28 (1H).

The in vitro activity of the compounds of the present invention can be demonstrated in the following assays:

Biochemical Assay 1: hK-RasG12C Interaction Assay with hSOS1

This assay quantifies the equilibrium interaction of human SOS1 (hSOS1) with human K-Ras^{G12C} (hK-RasG12C). Detection of the interaction is achieved by measuring homogenous time-resolved fluorescence resonance energy transfer (HTRF) from antiGST-Europium (FRET donor) bound to GST-K-RasG12C to anti-6His-XL665 bound to His-tagged hSOS1 (FRET-acceptor).

The assay buffer containes 5 mM HEPES pH 7.4 (Applichem), 150 mM NaCl (Sigma), 10 mM EDTA (Promega), 1 mM DTT (Thermofisher), 0.05% BSA Fraction V, pH 7.0, (ICN Biomedicals), 0.0025% (v/v) Igepal (Sigma) and 100 mM KF (FLUKA).

The expression and purification of N-terminal GST-tagged hK-RasG12C and N-terminal His-tagged hSOS1 is described below. Concentrations of protein batches used are optimized to be within the linear range of the HTRF signal. A Ras working solution is prepared in assay buffer containing typically 10 nM GST-hK-RasG12C and 2 nM antiGST-Eu(K) (Cisbio, France). A SOS1 working solution is prepared in assay buffer containing typically 20 nM His-hSOS1 and 10 nM anti-6His-XL665 (Cisbio, France). An inhibitor control solution is prepared in assay buffer containing 10 nM anti-6His-XL665 without hSOS1.

Fifty nl of a 100-fold concentrated solution of the test compound in DMSO are transferred into a black microtiter test plate (384 or 1536, Greiner Bio-One, Germany). For this, either a Hummingbird liquid handler (Digilab, Mass., USA) or an Echo acoustic system (Labcyte, Calif., USA) is used.

All steps of the assay are performed at 20° C. A volume of 2.5 μl of the Ras working solution is added to all wells of the test plate using a Multidrop dispenser (Thermo Labsystems). After 2 min preincubation, 2.5 μl of the SOS1 working solution are added to all wells except for those wells at the side of the test plate that are subsequently filled with 2.5 μl of the inhibitor control solution. After 60 min incubation the fluorescence is measured with a Pherastar (BMG, Germany) using the HTRF module (excitation 337 nm, emission 1: 620nm, emission 2: 665 nm).

The ratiometric data (emission 2 divided by emission 1) are normalized using the controls (DMSO=0% inhibition, inhibition control wells with inhibitor control solution=100% inhibition). Compounds are tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0.073 nM). IC50 values are calculated by 4-Parameter fitting using a commercial software package (Genedata Screener, Switzerland).

Biochemical Assay 2: hK-RasG12C Activation Assay by hSOS1 at High GTP Concentration

This assay quantifies human SOS1-mediated nucleotide exchange of human K-Ras^{G12C} (hK-RasG12C) preloaded with a fluorescent GTP-analog and in presence of an excess of free GTP. Loaded hK-RasG12C generates a high HTRF-signal by energy transfer from antiGST-Terbium (FRET donor) bound to hK-Ras to the loaded fluorescent GDP analog (FRET-acceptor). hSOS1 activity exchanges the fluorescent GDP for non-fluorescent GTP and therefore leads to a reduction of the HTRF signal.

The fluorescent GDP-analog EDA-GDP-Dy647P1 (2′/3′-O-(2-Aminoethyl-carbamoyl)-guanosine-5′-diphosphate labelled with Dy647P1 (Dyomics GmbH, Germany)) is synthesized by Jena Biosciences GmbH (Germany) and supplied as a 1 mM aqueous solution.

The expression and purification of N-terminal GST-tagged human K-RasG12C and N-terminal His-tagged human SOS1 is described below. Concentrations of protein batches used are optimized to be within the linear range of the HTRF signal.

Preparation of GST-tagged hK-RasG12C loaded with fluorescent nucleotide is performed as follows: incubation of 11.5 μM hK-Ras^{G12C} with 5-fold excess GDP-Dy647 nucleotide (54 μM) in 500 μl NLS-buffer (RAS activation Kit Jena Bioscience, Kat. #PR-950) for 10 min at 37° C. Addition of 20 μl 1 M MgCl₂ (Sigma) to final 40 mM and store on ice. Purification into buffer (10 mM HEPES pH 7.4 (Applichem), 150 mM NaCl (Sigma), 5 mM MgCl₂ (Sigma)) by use of a PD-Minitrap desalting column (GE Healthcare). Concentration of 1 ml purified hK-Ras-GDP-Dy647 is approx. 4-5 μM.

The assay buffer containes 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

A Ras working solution is prepared in assay buffer containing typically 80 nM loaded GST-hK-RasG12C-EDA-GDP-Dy647P1 and 2 nM antiGST-Tb (Cisbio, France). A hSOS1 working solution is prepared in assay buffer containing typically 8 nM His-hSOS1 and 100 μM GTP (Jena Bioscience, Germany). An inhibitor control solution is prepared in assay buffer containing the same concentration of hSOS1 without GTP.

Alternatively, the inhibitor control solution is prepared by supplementing the hSOS1 working solution with 20 μM of 6,7-dimethoxy-N-[(1R)-1-(1-naphthyl)ethyl]quinazolin-4-amine which is used to calibrate the assay.

Fifty nl of a 100-fold concentrated solution of the test compound in DMSO are transferred into a black microtiter test plate (384 or 1536, Greiner Bio-One, Germany). For this, either a Hummingbird liquid handler (Digilab, Mass., USA) or an Echo acoustic system (Labcyte, Calif., USA) is used.

All steps of the assay are performed at 20° C. A volume of 2.5 μl of the Ras working solution is added to all wells of the test plate using a Multidrop dispenser (Thermo Labsystems). After 2 min preincubation, 2.5 μl of the hSOS1 working solution are added to all wells except for those wells at the side of the test plate that are subsequently filled with 2.5 μl of the inhibitor control solution. After 20 min incubation the fluorescence is measured with a Pherastar (BMG, Germany) using the HTRF module (excitation 337 nm, emission 1: 620 nm, emission 2: 665 nm).

The ratiometric data (emission 2 divided by emission 1) are normalized using the controls (DMSO=0% inhibition, inhibition control wells with inhibitor control solution=100% inhibition). Compounds are tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0.073 nM). IC50 values are calculated by 4-Parameter fitting using a commercial software package (Genedata Screener, Switzerland).

Biochemical Assay 3: hK-RasG12C Activation Assay by hSOS1

K-Ras is a small GTPase that can bind GDP and GTP. The guanine nucleotide exchange factor SOS1 catalyzes the activation of K-Ras by promoting the exchange of GDP to GTP. SOS1 binds to K-Ras-GDP thereby opening the GDP-binding pocket to facilitate GDP release. Rebinding of excess nucleotide leads to dissociation of the K-Ras-SOS1 intermediate complex leaving K-Ras loaded with the nucleotide.

This assay quantifies human SOS1- (hSOS1-) mediated loading of human K-Ras^{G12C}-GDP (hK-RasG12C-GDP) with a fluorescent GTP-analog. Detection of successful loading is achieved by measuring homogenous time-resolved fluorescence resonance energy transfer (HTRF) from antiGST-Terbium (FRET donor) bound to GST-hK-RasG12C (see below) to the loaded fluorescent GTP analog (FRET-acceptor).

The fluorescent GTP-analog EDA-GTP-Dy647P1 (2′/3′-O-(2-Aminoethyl-carbamoyl)-guanosine-5′-triphosphate labelled with Dy647P1 (Dyomics GmbH, Germany)) is synthesized by Jena Biosciences GmbH (Germany) and supplied as a 1mM aqueous solution.

The assay buffer containes 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

The expression and purification of N-terminal GST-tagged human K-RasG12C and N-terminal His-tagged hSOS1 is described below. Concentrations of protein batches used are optimized to be within the linear range of the HTRF signal. A hRas working solution is prepared in assay buffer containing typically 100 nM GST-hK-RasG12C and 2 nM antiGST-Tb (Cisbio, France). A hSOS1 working solution is prepared in assay buffer containing typically 20nM hSOS1 and 200 nM EDA-GTP-Dy647P1. An inhibitor control solution is prepared in assay buffer containing 200 nM EDA-GTP-Dy647P1 without hSOS1.

Fifty nl of a 100-fold concentrated solution of the test compound in DMSO are transferred into a black microtiter test plate (384 or 1536, Greiner Bio-One, Germany). For this, either a Hummingbird liquid handler (Digilab, Mass., USA) or an Echo acoustic system (Labcyte, Calif., USA) is used.

All steps of the assay are performed at 20° C. A volume of 2.5 μl of the hRas working solution is added to all wells of the test plate using a Multidrop dispenser (Thermo Labsystems). After 10 min preincubation, 2.5 μl of the hSOS1 working solution are added to all wells except for those wells at the side of the test plate that are subsequently filled with 2.5 μl of the inhibitor control solution. After 30 min incubation the fluorescence is measured with a Pherastar (BMG, Germany) using the HTRF module (excitation 337 nm, emission 1: 620 nm, emission 2: 665 nm).

The ratiometric data (emission 2 divided by emission 1) are normalized using the controls (DMSO=0% inhibition, inhibition control wells with inhibitor control solution=100% inhibition). Compounds are tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0.073 nM). IC50 values are calculated by 4-Parameter fitting using a commercial software package (Genedata Screener, Switzerland).

Biochemical Assay 4: hK-RasG12C Activation Assay by hSOS2

This assay quantifies hSOS1-mediated loading of hK-Ras^{G12C}-GDP (hK-RasG12C-GDP) with a fluorescent GTP-analog. Detection of successful loading is achieved by measuring homogenous time-resolved fluorescence resonance energy transfer (HTRF) from antiGST-Terbium (FRET donor) bound to GST-hK-RasG12C to the loaded fluorescent GTP analog (FRET-acceptor).

The fluorescent GTP-analog EDA-GTP-Dy647P1 (2′/3′-O-(2-Aminoethyl-carbamoyl)-guanosine-5′-triphosphate labelled with Dy647P1 (Dyomics GmbH, Germany)) is synthesized by Jena Biosciences GmbH (Germany) and supplied as a 1 mM aqueous solution.

The assay buffer containes 10 mM HEPES pH 7.4 (Applichem), 150 mM NaCl (Sigma), 5 mM MgCl₂ (Sigma), 1 mM DTT (Thermofisher), 0.05% BSA Fraction V, pH 7.0, (ICN Biomedicals), 0.0025% (v/v) Igepal (Sigma).

The expression and purification of N-terminal GST-tagged hK-RasG12C and N-terminal His-tagged hSOS1 is described below. Concentrations of protein batches used are optimized to be within the linear range of the HTRF signal. A hRas working solution is prepared in assay buffer containing typically 100 nM GST-hK-RasG12C and 2 nM antiGST-Tb (Cisbio, France). A hSOS2 working solution is prepared in assay buffer containing typically 20 nM hSOS2 and 200 nM EDA-GTP-Dy647P1. An inhibitor control solution is prepared in assay buffer containing 200 nM EDA-GTP-Dy647P1 without hSOS.

Fifty nl of a 100-fold concentrated solution of the test compound in DMSO are transferred into a black microtiter test plate (384 or 1536, Greiner Bio-One, Germany). For this, either a Hummingbird liquid handler (Digilab, Mass., USA) or an Echo acoustic system (Labcyte, Calif., USA) is used.

All steps of the assay are performed at 20° C. A volume of 2.5 μl of the hRas working solution is added to all wells of the test plate using a Multidrop dispenser (Thermo Labsystems). After 10 min preincubation, 2.5 μl of the hSOS1 working solution are added to all wells except for those wells at the side of the test plate that are subsequently filled with 2.5 μl of the inhibitor control solution. After 30 min incubation the fluorescence is measured with a Pherastar (BMG, Germany) using the HTRF module (excitation 337 nm, emission 1: 620 nm, emission 2: 665 nm).

The ratiometric data (emission 2 divided by emission 1) are normalized using the controls (DMSO =0% inhibition, inhibition control wells with inhibitor control solution=100% inhibition). Compounds are tested in duplicates at up to 11 concentrations (for example 20 μM, 5.7 μM, 1.6 μM, 0.47 μM, 0.13 μM, 38 nM, 11 nM, 3.1 nM, 0.89 nM, 0.25 nM and 0.073 nM). IC50 values are calculated by 4-Parameter fitting using a commercial software package (Genedata Screener, Switzerland).

EGFR Kinase Assay

EGFR inhibitory activity of compounds of the present invention is quantified employing the TR-FRET based EGFR assay as described in the following paragraphs.

Epidermal Growth Factor Receptor (EGFR) affinity purified from human carcinoma A431 cells (Sigma-Aldrich, #E3641) is used as kinase. As substrate for the kinase reaction the biotinylated peptide biotin-Ahx-AEEEEYFELVAKKK (C-terminus in amid form) is used which can be purchased e.g. form the company Biosyntan GmbH (Berlin-Buch, Germany).

For the assay 50 nL of a 100 fold concentrated solution of the test compound in DMSO is pipetted into a black low volume 384 well microtiter plate (Greiner Bio-One, Frickenhausen, Germany), 2 μL of a solution of EGFR in aqueous assay buffer [50 mM Hepes/HCl pH 7.0, 1 mM MgCl₂, 5 mM MnCl₂, 0.5 mM activated sodium ortho-vanadate, 0.005% (v/v) Tween-20] are added and the mixture is incubated for 15 min at 22° C. to allow pre-binding of the test compounds to the enzyme before the start of the kinase reaction. Then the kinase reaction is started by the addition of 3μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture is incubated for a reaction time of 20 min at 22° C. The concentration of EGFR is adjusted depending of the activity of the enzyme lot and is chosen appropriate to have the assay in the linear range, typical concentration are about 3 U/ml. The reaction is stopped by the addition of 5 μl of a solution of HTRF detection reagents (0.1 μM streptavidine-XL665 [Cis Biointernational] and 1 nM PT66-Tb-Cryptate, an terbium-cryptate labelled anti-phospho-tyrosine antibody from Cis Biointernational [instead of the PT66-Tb-cryptate PT66-Eu-Chelate from Perkin Elmer can also be used]) in an aqueous EDTA-solution (80 mM EDTA, 0.2% (w/v) bovine serum albumin in 50 mM HEPES pH 7.5).

The resulting mixture is incubated 1 h at 22° C. to allow the binding of the biotinylated phosphorylated peptide to the streptavidine-XL665 and the PT66-Eu-Chelate. Subsequently the amount of phosphorylated substrate is evaluated by measurement of the resonance energy transfer from the PT66-Tb-Cryptate to the streptavidine-XL665. Therefore, the fluorescence emissions at 620 nm and 665 nm after excitation at 337 nm are measured in a HTRF reader, e.g. a Pherastar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.072 nM (e.g. 20 μM, 5.7 μM, 1.6 μM, 0.47 μM, 0.13 μM, 38 nM, 11 nM, 3.1 nM, 0.89 nM, 0.25 nM and 0.072 nM, the dilution series are prepared separately before the assay on the level of the 100 fold concentrated solutions in DMSO by serial dilutions, the exact concentrations may vary depending on the pipettor used) in duplicate values for each concentration and IC50 values are calculated by a 4 parameter fit.

Cellular Assays

3D-Softagar MiaPaca-2 (ATCC CRL-1420) and NCI-H1792 (ATCC CRL-5895)

Day 1: Softagar (Select Agar, Invitrogen, 3% in ddH2O autoclaved) is boiled and tempered at 48° C. Medium (MiaPaca-2: DMEM/Ham's F12; [Biochrom; # FG 4815, with stable Glutamine] 10% FCS and 2.5% Horse Serum, H1792: RPMI 1640; [Biochrom; # FG 1215, with stable Glutamine and 10%FCS]) is tempered to 37° C.; Agar (3%) is diluted 1:5 in medium (=0.6%) and 50 μl/well plated into 96 well plates (Corning, #3904), wait at room temperature until the agar is solid. 3% agar is diluted to 0.25% in medium (1:12 dilution) and tempered at 42° C. Cells are trypsinized, counted and tempered at 37° C.; cells (MiaPaCa-2: 125-150, NCI-H1792: 1000) are resuspended in 100 μl 10.25% Agar and plated. Wait at room temperature until the agar is solid. Overlay wells with 50 μl medium. Plate sister wells in separate plate for time zero determination. All plates are incubated overnight 37° C. and 5% CO2.

Day 2: Measurement of time zero values: Add 40 μl Cell Titer 96 Aqueous Solution (Promega) per well, (light sensitive) and incubate in the dark at 37° C. and 5% CO2. Absorption is measured at 490 nm and reference wavelength 660 nm. DMSO-prediluted test compounds are added with HP Dispenser to a final DMSO concentration of 0.3%.

Day 10: Measurement of test compound and control treated wells with Cell Titer 96 AQueous according to time zero. The IC50 values were determined using the four parameter fit.

Active RAS in Calu-1 Cells (CLS 300141)

40.000 Calu-1 cells are seeded in 96 well plate (NUNC161093) for 48 h at 37° C./5% CO2 (10% FBS (S0615), DMEM/Ham's F-12 (Biochrom; #FG 4815), 2 mM L-Glutamine). After that, medium is changed to FBS-free medium and the cells were incubated for further 24h at 37° C./5% CO2. Cells are treated with varying concentrations of DMSO-prediluted test compounds (final 0.1%) for 30 min at 37° C./5% CO2. Supernatant with test compounds is discarded and, after that, treated cells are stimulated with 100 ng/ml EGF (Sigma #E9644, diluted in serum free medium) for 3 minutes. Cells were treated with lysis buffer and all next steps were performed on ice according to the supplier's manual of G-LISA Kit (Cytoskeleton BK131, Ras Activation Assay). Finally, the content of active Ras is measured by detecting the absorbance at 490 nm (Tecan Sunrise). The value of EGF-stimulated cells is set as 100%, whereas the value of untreated cells is set as 0%. The IC50 values were determined using the four parameter fit.

Active Ras in Hela Cells (ATCC CCL-2)

30.000 Hela cells are seeded in 96 well plate for 96 h at 37° C. (10% FBS, DMEM/Ham's F-12, 2 mM L-Glutamine). After that, medium is changed in to FBS-free medium for 24 h. Cells are treated with varying concentrations of test compounds for 30 min. After that, treated cells are stimulated with 100 ng/ml EGF for 2 minutes. Cells are treated with lysis buffer and all next steps are performed on ice according to the supplier's manual of G-LISA Kit (Cytoskeleton BK131, Ras Activation Assay). Finally, the content of active Ras is measured by detecting the absorbance at 490 nm. The value of EGF-stimulated cells is set as 100%, whereas the value of untreated cells is set as 0%. The results given as % reflecting the inhibition of formation of active Ras compared to control.

The IC50 values are determined using the four parameter fit.

pERK HTRF in MOLM-13 (DMSZ ACC 554)

10000 MOLM-13 cells are seeded in HTRF 384 well low volume plate (Greiner bio-one #784080) in medium (RPMI 1640+10% FCS). After 24 hours, cells are treated with varying concentrations of test compounds for 1h. Next steps are performed to the supplier's manual Advanced phospho-ERK1/2 (#64AERPEH) Cisbio one-plate assay protocol. The content of pERK is measured with PHERAstar HTRF protocol, calculated Ratio*1000.

The calculated ratio of DMSO-treated cells is set as 100% and the calculated ratio of negative control is set as 0% (maximum possible effect). The results given as IC50 reflecting the inhibition of formation of pERK compared to DMSO control and negative control and normalized according to cell number.

The IC50 values are determined by means of a 4 parameter fit.

pERK HTRF in Calu-1 (CLS 300141)

5000 Calu-1 cells are seeded in HTRF 384we11 low volume plate (Greiner bio-one #784080) in medium (McCoy's 5A+10% FCS). After 24 hours, cells are treated with varying concentrations of test compounds for 24 h. Next steps are performed to the supplier's manual Advanced phospho-ERK1/2 (#64AERPEH) Cisbio one-plate assay protocol. The content of pERK is measured with PHERAstar HTRF protocol, calculated Ratio*1000.

The calculated ratio of DMSO-treated cells is set as 100% and the calculated ratio of negative control is set as 0% (maximum possible effect). The results given as IC50 reflecting the inhibition of formation of pERK compared to DMSO control and negative control and normalized according to cell number.

The IC50 values are determined by means of a 4 parameter fit.

pERK HTRF in K-562 (ATCC CCL-243)

10000 K-562 cells are seeded in HTRF 384we11 low volume plate (Greiner bio-one #784075) in medium (RPMI 1640+10% FCS) and treated with varying concentrations of test compounds for 1 h. Next steps are performed to the supplier's manual Advanced phospho-ERK1/2 (#64AERPEH) Cisbio one-plate assay protocol. The content of pERK is measured with PHERAstar HTRF protocol, calculated Ratio*1000.

The calculated ratio of DMSO-treated cells is set as 100% and the calculated ratio of negative control is set as 0% (maximum possible effect). The results given as IC50 reflecting the inhibition of formation of pERK compared to DMSO control and negative control and normalized according to cell number.

The IC50 values are determined by means of a 4 parameter fit.

pERK Assay in NCI-H358 Cells (ATCC CRL-5807) for Combination Experiments

5000 NCI-H358 cells are seeded in HTRF 384we11 low volume plate (Greiner bio-one #784080) in medium (RPMI+10% FCS). After 24 h, cells are treated for 1 h with component A and with component B for single compound treatments (final concentration ranges covering the expected IC50 values), and in nine different fixed-ratio combinations of compound A (D1) and compound B (D2) (0.9×D1+0.1×D2, 0.8×D1+0.2×D2, 0.7×D1+0.3×D2, 0.6×D1+0.4×D2, 0.5×D1+0.5×D2, 0.4×D1+0.6×D2, 0.3×D1+0.7×D2, 0.2×D1+0.8×D2, 0.1×D1+0.9×D2) using a Tecan HP digital dispenser.

Next steps are performed to the supplier's manual Advanced phospho-ERK1/2 (#64AERPEH) Cisbio one-plate assay protocol. The content of pERK is measured with PHERAstar HTRF protocol, calculated Ratio*1000.

IC50 values (inhibitory concentration at 50% of maximal effect) are determined by means of a 4 parameter fit on measurement data which are normalized to vehicle (DMSO) treated cells (=100%) and measurement readings taken immediately before compound exposure (=0%). IC50 isobolograms are plotted with the actual concentrations of the two compounds on the x- and y-axis, and the combination index (CI) is calculated according to the median-effect model of Chou-Talalay (Chou T. C. 2006 Pharmacol. Rev.). A CI of <0.8 is defined as more than additive (synergistic) interaction, and a CI of >1.2 is defined as antagonistic interaction.

P-EGFR Assay (In-Cell Western) in Hela Cells (ATCC CCL-2)

After stimulation with EGF, the EGF receptor autophosphorylates at Y1173. In-cell Western assay simultaneously detect two targets at 700 and 800 nm using two spectrally distinct near-infrared dyes. With a specific antibody, phosphorylated EGFR can be quantified and the samples can be normalized with total EGFR antibody parallel.

25000 Hela cells are seeded in 96we11 plate (NUNC161093) for 24 h at 37° C./5%CO2 (10% FBS (S0615), DMEM/Ham's F-12 (Biochrom; #FG 4815), 2 mM L-Glutamine). After that, medium is changed to FBS-free medium and the cells are incubated for further 24h at 37° C./5% CO2.

Cells are treated with varying concentrations of DMSO-prediluted test compounds (final 0.1%) for 30 minutes and finally with 100 ng/ml EGF (Sigma#E9644, diluted in serum free medium) for 2 minutes.

Cells are treated according the manual of EGFR Near Infrared In-Cell ELISA Kit (Pierce #62210). If not specified, all buffers and antibodies are part of this kit.

Cells are fixed with 4% formaldehyde, washed twice with 100μl per well with TRIS-buffered saline with Surfact-Amps 20, permeabilized with 100μl TRIS-buffered saline with Surfact-Amps X-100, wash again with 100μl TRIS-buffered saline, and finally 200μl blocking buffer are added for 60 minutes at room temperature. Fixed and washed cells are incubated with primary antibody mix (P-EGFR; EGFR) overnight at 2-8° C. After washing with 100μl TRIS-buffered saline with Surfact-Amps 20, secondary IRDye-labeled antibody mix (DyLight 800 Goat Anti-Rabbit IgG, Pierce SA5-35571; DyLight 680 Goat Anti-Mouse IgG, Pierce 35518) is added for 1 h at room temperature and washed again. Plates are scanned with LiCor Odyssey Infrared Imager at 800 nm for P-EGFR and at 700 nm for total EGFR. The quotient of 800 nm and 700 nm for EGF only treated cells is set as 100% and the quotient of 800 nm and 700 nm of untreated cells is set as 0%. The IC50 values are determined using the four parameter fit.

pERK Assay in NCl-H358 Cells (ATCC CRL-5807) for combination experiments

NCl-H358 human non-small cell lung tumor cells (ATCC CRL-5807) are propagated in a humidified 37° C. incubator in RPMI1640 growth medium (Thermo Fisher Gibco, #61870-010) supplemented with 10% fetal calf serum (Biochrom, #S 0615). For analysis of combination effects between compound A and compound B, cells are plated in 384-well plates (Greiner bio-one, #784080) at a density of 20,000 cells per well in 8 microL of growth medium supplemented with 10% fetal calf serum. After 24 h, cells are treated with component A and with component B for single compound treatments (final concentration ranges covering the expected IC50 values), and in nine different fixed-ratio combinations of compound A (D1) and compound B (D2) (0.9×D1+0.1×D2, 0.8×D1+0.2×D2, 0.7×D1+0.3×D2, 0.6×D1+0.4×D2, 0.5×D1+0.5×D2, 0.4×D1+0.6×D2, 0.3×D1+0.7×D2, 0.2×D1+0.8×D2, 0.1×D1+0.9×D2) using a Tecan HP digital dispenser. The cells are incubated for 60 minutes at 37° C. 4 microL/well of a freshly prepared solution of 0.6 nanog/microL of epidermal growth factor (Sigma, #E9644) in RPMI1640 medium are added using a Thermo Fisher Multidrop device (final concentration 200 nanog/milliL). The cells are incubated for another 3 minutes immediately followed by the detection of total ERK1/2 and phosphorylated ERK1/2 at positions Thr202/Tyr204 using commercial HTRF detection kits (Cisbio: total ERK1/2, 64NRKPEG; phospho-ERK1/2, 64AERPEH) and a PHERAstar microplate reader device (BMG Labtech). Cell lysis and detection are performed according to the manufacturer's recommendations. The ratio of phosphorylated ERK1/2 to total ERK1/2 protein are calculated and IC50 values (inhibitory concentration at 50% of maximal effect) are determined by means of a 4 parameter fit on measurement data which are normalized to vehicle (DMSO) treated cells (=100%). IC50 isobolograms are plotted with the actual concentrations of the two compounds on the x- and y-axis, and the combination index (CI) is calculated according to the median-effect model of Chou-Talalay (Chou T. C. 2006 Pharmacol. Rev.). A CI of <0.8 is defined as more than additive (synergistic) interaction, and a CI of >1.2 is defined as antagonistic interaction.

TABLE 1
IC₅₀ values of some examples in the K-RasG12C - SOS interaction assay, in K-RasG12C
activation by SOS, in K-Ras activation by SOS high GTP and in K-Ras-wt activation by SOS

| | K-RasG12C- SOS | K-RasG12C | K-Ras activation by | K-Ras-wt |
|---|---|---|---|---|
| | interaction assay | activation by SOS | SOS high GTP (off- | activation by SOS |
| | IC50 - [mol/l] | IC50 - [mol/l] | HTRF) IC50 - [mol/l] | IC50 - [mol/l] |
| Structure | (mean) | (mean) | (mean) | (mean) |
| 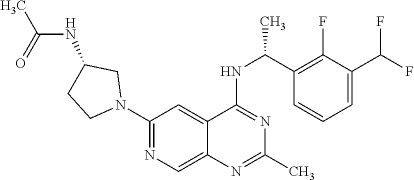 [SMILES: CC(=O)N[C@H]1CCN(c2cc3c(N[C@H](C)c4cccc(C(F)F)c4F)nc(C)nc3cn2)C1] | 7.98 E−8 | 1.44 E−7 | 1.97 E−7 | 1.01 E−7 |
| 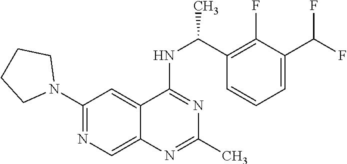 [SMILES: Cc1nc(N[C@H](C)c2cccc(C(F)F)c2F)c2cc(N3CCCC3)ncc2n1] | 1.85 E−7 | 2.51 E−7 | 3.20 E−7 | 1.72 E−7 |
| 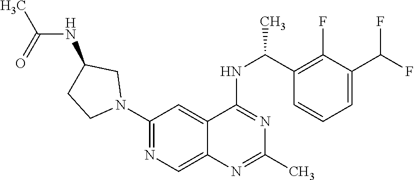 [SMILES: CC(=O)N[C@@H]1CCN(c2cc3c(N[C@H](C)c4cccc(C(F)F)c4F)nc(C)nc3cn2)C1] | 7.73 E−8 | 1.10 E−7 | 1.87 E−7 | 9.23 E−8 |
| 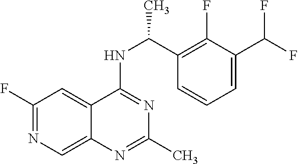 [SMILES: Cc1nc(N[C@H](C)c2cccc(C(F)F)c2F)c2cc(F)ncc2n1] | >2.00 E−5 | >2.00 E−5 | >2.00 E−5 | >2.00 E−5 |
| 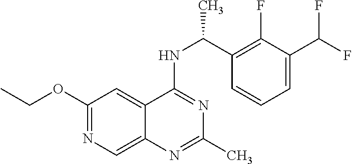 [SMILES: CCOc1cc2c(N[C@H](C)c3cccc(C(F)F)c3F)nc(C)nc2cn1] | 1.34 E−7 | 3.57 E−7 | 8.51 E−7 | 3.08 E−7 |
| 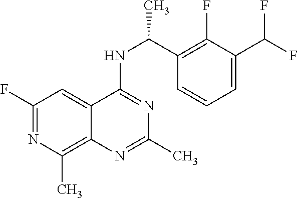 [SMILES: Cc1nc(N[C@H](C)c2cccc(C(F)F)c2F)c2cc(F)nc(C)c2n1] | >2.00 E−5 | >2.00 E−5 | not determined yet | >2.00 E−5 |
| 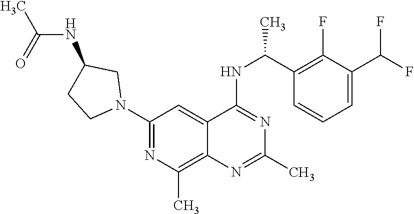 [SMILES: CC(=O)N[C@@H]1CCN(c2cc3c(N[C@H](C)c4cccc(C(F)F)c4F)nc(C)nc3c(C)n2)C1] | 5.68 E−8 | 2.09 E−7 | 2.55 E−7 | 8.32 E−8 |

As exemplified in table 1, the compounds of the present invention inhibit the binding of hSOS1 to hKRAS, which was measured in the biochemical hK-RasG12C -hSOS1 interaction assay (assay 1). The ability to inhibit the hKRAS-hSOS1 interaction results in the inhibition of hKRAS activation by the compounds, as measured in biochemical assay 3, which quantifies the hSOS1-mediated nucleotide exchange from hK-RasG12C-GDP to hK-RasG12C loaded with a fluorescent GTP-analog. Furthermore, the compounds of the present invention show the ability to inhibit the nucleotide exchange reaction catalyzed by hSOS1 in the presence of a high concentration of 50 μM GTP, as measured in assay 2. This ability increases the chance that the compounds will be able to inhibit hSOS1 mediated hKRAS-activation inside cells, where high GTP concentrations are present. The chemical structure of the compounds of the present invention is similar to known inhibitors of EGFR-kinase. As shown in table 1, most compounds are inactive against EGFR-kinase up to the highest concentration measured in the assay (>20 μM).

The assay data of the large number of compounds in table 1 gives evidence that compounds which have a pharmacological profile as tested according to assays 1 to 3 and as described in the preceding paragraph will be generally useful to inhibit hSOS1 mediated hKRAS-activation inside cells, where high GTP concentrations are present and activity against EGFR-kinase up to highest concentrations (>20 μM) will not be measured in the assay.

Therefore an even further aspect of the present invention refers to the use of a compound which inhibits the binding of hSOS1 to human H- or N- or K-RAS including their clinically known mutations and which inhibits the nucleotide exchange reaction catalyzed by hSOS1 in the presence of a concentration of 20 μM or lower, but which is substantially inactive against EGFR-kinase at concentrations of 20 μM or lower for the preparation of a medicament for the treatment or prophylaxis of a hyperproliferative disorder.

Particularly this aspect refers to the use of a compound which inhibits the binding of hSOS1 specifically to hK-RasG12C protein and which inhibits the nucleotide exchange reaction catalyzed by hSOS1 in the presence of a concentration of 20 μM or lower, but which is substantially inactive against EGFR-kinase at concentrations of 20 μM or lower for the preparation of a medicament for the treatment or prophylaxis of a hyperproliferative disorder.

Expression of hK-RasG12C, hSOS1, hSOS1_12 and hSOS2 in E. coli:

The applied DNA expression constructs encoding the following protein sequences and its corresponding DNA sequences were optimized for expression in E. coli and synthesized by the GeneArt Technology at Life Technologies:

Human K-Ras (P01116-2):

hK-RasG12C (amino acid 1-169)

Human SOS1 (Q07889):

hSOS1 (amino acid 564-1049)

hSOS1_12: (amino acid 564-1049 which is fused at its N-terminus with the amino acid sequence GAMA

Human SOS2 (Q07890):

hSOS2 (amino acid 564-1043)

These expressions construct additionally encoded att-site sequences at the 5′and 3′ ends for subcloning into various destination vectors using the Gateway Technology as well as a TEV (Tobacco Etch Virus) protease site for proteolytic cleavage of tag sequences. The applied destination vectors were: pD-ECO1 (an in-house derivate of the pET vector series from Novagen with ampicillin resistance gene) which provides an N-terminal fusion of a GST-tag to the integrated gene of interest. pD-ECO5 (also an in-house derivative of the pET vector series with ampicillin resistance gene) which provides a N-terminal fusion of a His10-tag to the integrated gene. To generate the final expression vectors the expression construct of hK-Ras_G12C was cloned into pD-ECO1. hSOS1, hSOS1_12 as well as hSOS2 were cloned into pD-ECOS. The resulting expression vectors were termed pD-ECO1_hK-RasG12C, pD-ECO5_hSOS1, pD-ECO5_hSOS1_12, pD-ECOS_hSOS2

Sequences:
GST-hK-RasG12C (G12C mutation according
to numbering in P01116-2)
MSPILGYWKI KGLVQPTRLL LEYLEEKYEE
HLYERDEGDK WRNKKFELGL EFPNLPYYID
GDVKLTQSMA IIRYIADKHN MLGGCPKERA
EISMLEGAVL DIRYGVSRIA YSKDFETLKV
DFLSKLPEML KMFEDRLCHK TYLNGDHVTH
PDFMLYDALD VVLYMDPMCL DAFPKLVCFK
KRIEAIPQID KYLKSSKYIA WPLQGWQATF
GGGDHPPKSD PITSLYKKAG SDYDIPTTEN
LYFQGMTEYK LVVVGACGVG KSALTIQLIQ
NHFVDEYDPT IEDSYRKQVV IDGETCLLDI
LDTAGQEEYS AMRDQYMRTG EGFLCVFAIN
NTKSFEDIHH YREQIKRVKD SEDVPMVLVG
NKCDLPSRTV DTKQAQDLAR SYGIPFIETS
AKTRQGVDDA FYTLVREIRK HKEK
His10-hSOS1
MGHHHHHHHH HHSSGHIEGR HMLETSLYKK
AGSDYDIPTT ENLYFQGEEQ MRLPSADVYR
FAEPDSEENI IFEENMQPKA GIPIIKAGTV
IKLIERLTYH MYADPNFVRT FLTTYRSFCK
PQELLSLIIE RFEIPEPEPT EADRIAIENG
DQPLSAELKR FRKEYIQPVQ LRVLNVCRHW
VEHHFYDFER DAYLLQRMEE FIGTVRGKAM
KKWVESITKI IQRKKIARDN GPGHNITFQS
SPPTVEWHIS RPGHIETFDL LTLHPIEIAR
QLTLLESDLY RAVQPSELVG SVWTKEDKEI
NSPNLLKMIR HTTNLTLWFE KCIVETENLE
ERVAVVSRII EILQVFQELN NFNGVLEVVS
AMNSSPVYRL DHTFEQIPSR QKKILEEAHE
LSEDHYKKYL AKLRSINPPC VPFFGIYLTN
ILKTEEGNPE VLKRHGKELI NFSKRRKVAE
ITGEIQQYQN QPYCLRVESD IKRFFENLNP
MGNSMEKEFT DYLFNKSLEI EPRNPKPLPR
FPKKYSYPLK SPGVRPSNPR PGT
His10-hSOS1_12
MGHHHHHHHH HHSSGHIEGR HMLETSLYKK
AGSDYDIPTT ENLYFQGAMA EEQMRLPSAD
VYRFAEPDSE ENIIFEENMQ PKAGIPIIKA
GTVIKLIERL TYHMYADPNF VRTFLTTYRS
FCKPQELLSL IIERFEIPEP EPTEADRIAI
ENGDQPLSAE LKRFRKEYIQ PVQLRVLNVC
RHWVEHHFYD FERDAYLLQR MEEFIGTVRG
KAMKKWVESI TKIIQRKKIA RDNGPGHNIT
FQSSPPTVEW HISRPGHIET FDLLTLHPIE
IARQLTLLES DLYRAVQPSE LVGSVWTKED
KEINSPNLLK MIRHTTNLTL WFEKCIVETE
NLEERVAVVS RIIEILQVFQ ELNNFNGVLE
VVSAMNSSPV YRLDHTFEQI PSRQKKILEE
AHELSEDHYK KYLAKLRSIN PPCVPFFGIY
LTNILKTEEG NPEVLKRHGK ELINFSKRRK
VAEITGEIQQ YQNQPYCLRV ESDIKRFFEN
LNPMGNSMEK EFTDYLFNKS LEIEPRNPKP
LPRFPKKYSY PLKSPGVRPS NPRPGT
hSOS1_12 (tag-free)
GAMAEEQMRL PSADVYRFAE PDSEENIIFE
ENMQPKAGIP IIKAGTVIKL IERLTYHMYA
DPNFVRTFLT TYRSFCKPQE LLSLIIERFE
IPEPEPTEAD RIAIENGDQP LSAELKRFRK
EYIQPVQLRV LNVCRHWVEH HFYDFERDAY
LLQRMEEFIG TVRGKAMKKW VESITKIIQR
KKIARDNGPG HNITFQSSPP TVEWHISRPG
HIETFDLLTL HPIEIARQLT LLESDLYRAV
QPSELVGSVW TKEDKEINSP NLLKMIRHTT
NLTLWFEKCI VETENLEERV AVVSRIIEIL
QVFQELNNFN GVLEVVSAMN SSPVYRLDHT
FEQIPSRQKK ILEEAHELSE DHYKKYLAKL
RSINPPCVPF FGIYLTNILK TEEGNPEVLK
RHGKELINFS KRRKVAEITG EIQQYQNQPY
CLRVESDIKR FFENLNPMGN SMEKEFTDYL
FNKSLEIEPR NPKPLPRFPK KYSYPLKSPG
VRPSNPRPGT
His10-hSOS2
MGHHHHHHHH HHSSGHIEGR HMLETSLYKK
AGSDYDIPTT ENLYFQGPLR LPSPEVYRFV
VKDSEENIVF EDNLQSRSGI PIIKGGTVVK
LIERLTYHMY ADPNFVRTFL TTYRSFCKPQ
ELLSLLIERF EIPEPEPTDA DKLAIEKGEQ
PISADLKRFR KEYVQPVQLR ILNVFRHWVE
HHFYDFERDL ELLERLESFI SSVRGKAMKK
WVESIAKIIR RKKQAQANGV SHNITFESPP
PPIEWHISKP GQFETFDLMT LHPIEIARQL
TLLESDLYRK VQPSELVGSV WTKEDKEINS
PNLLKMIRHT TNLTLWFEKC IVEAENFEER
VAVLSRIIEI LQVFQDLNNF NGVLEIVSAV
NSVSVYRLDH TFEALQERKR KILDEAVELS
QDHFKKYLVK LKSINPPCVP FFGIYLTNIL
KTEEGNNDFL KKKGKDLINF SKRRKVAEIT
GEIQQYQNQP YCLRIEPDMR RFFENLNPMG
SASEKEFTDY LFNKSLEIEP RNCKQPPRFP
RKSTFSLKSP GIRPNTG

E. coli Expression:

The expression vectors were transformed into E. coli strain BL21 (DE3). Cultivation of the transformed strains for expression was done in 10 L and 1 L fermenter.

The cultures were grown in Terrific Broth media (MP Biomedicals, Kat. #113045032) with 200 ug/mL ampicillin at a temperature of 37° C. to a density of 0.6 (OD600), shifted to a temperature of 27° C. (for hK-Ras expression vectors) or 17° C. (for hSOS expression vectors), induced for expression with 100 mM IPTG and further cultivated for 24 hours.

Purification

After cultivation the transformed E. coli were harvested by centrifugation and the resulting pellet was suspended in a lysis buffer (see below) and lysed by passing three-times through a high pressure device (Microfluidics). The lysate was centrifuged (49000g, 45 min, 4° C.) and the supernatant used for further purification.

An Äkta chromatography system was used for all further chromatography steps.

Purification of GST-hK-RasG12C for Biochemical Assays

E. coli culture (transformed with pD-ECO1_hK-RasG12C) from a 10 L fermenter was lysed in lysis buffer (50 mM Tris HCl 7.5, 500 mM NaCl,1 mM DTT, 0.5% CHAPS, Complete Protease Inhibitor Cocktail-(Roche)). As a first chromatography step the centrifuged lysate was incubated with 50 mL Glutathione Agarose 4B (Macherey-Nagel; 745500.100) in a spinner flask (16 h, 10° C.). The Glutathione Agarose 4B loaded with protein was transferred to a chromatography column connected to an Äkta chromatography system. The column was washed with wash buffer (50 mM Tris HCl 7.5, 500 mM NaCl, 1 mM DTT) and the bound protein eluted with elution buffer (50 mM Tris HCl 7.5, 500 mM NaCl, 1 mM DTT, 15 mM Glutathione). The main fractions of the elution peak (monitored by OD280) were pooled.

For further purification by size-exclusion chromatography the above eluate volume was applied to a column Superdex 200 HR prep grade (GE Healthcare) and the resulting peak fractions of the eluted fusion protein were collected. The final yield of hK-RasG12C was about 50 mg purified fusion protein per L culture and the final product concentration was about 1 mg/mL. Native mass spectrometry analyses of the final purified K-RasG12C demonstrated its homogeneous load with GDP.

Purification of His10-hSOS1 and His10-hSOS2 for Biochemical Assays

E. coli transformed with pD-ECO5_hSOS1 or pD-ECO5_hSOS2 were cultured and induced in a fermenter, harvested and lysed in lysis buffer (25 mM Tris HCl 7.5, 500 mM NaCl, 20 mM Imidazol, Complete EDTA-free (Roche)). For immobilized metal ion affinity chromatography (IMAC) the centrifuged lysate (50 000×g, 45 min, 4° C.) was incubated with 30 mL Ni-NTA (Macherey-Nagel; #745400.100) in a spinner flask (16 h, 4° C.) and subsequently transferred to a chromatography column connected to an Äkta chromatography system. The column was rinsed with wash buffer (25 mM Tris HCl 7.5, 500 mM NaCl, 20 mM Imidazol) and the bound protein eluted with a linear gradient (0-100%) of elution buffer (25 mM Tris HCl 7.5, 500 mM NaCl, 300 mM Imidazol). The main fractions of the elution peak (monitored by OD280) containing homogenous His10-hSOS were pooled. The final yield of His10-hSOS1 was about 110 mg purified protein per L culture and the final product concentration was about 2 mg/mL. For His10-hSOS2 the final yield was 190 mg per L culture and the product concentration 6 mg/mL.

Purification of hSOS1_12

To produce tag-free hSOS1_12 the same process consisting of 4 chromatography steps applying an Äkta system was used as decribed here below for hSOS1.

His10-hSOS1_12 was expressed in E. coli transformed with pD-ECOS_hSOS1_12 as described above.

For IMAC the centrifuged lysate was directly applied to a 30 mL (or 50 mL) column with Ni-NTA (Macherey-Nagel) in an Äkta system, rinsed with wash buffer (25 mM Tris HCl 7.5, 500 mM NaCl, 20 mM Imidazol) and the bound protein was eluted with a linear gradient (0-100%) of elution buffer (25 mM Tris HCl 7.5, 500 mM NaCl, 300 mM Imidazol). The main fractions of the elution peak (monitored by OD280) were passed over a HiPrep Desalting column (GE; #17-5087-01) to change to the cleavage buffer (25 mM Tris HCl 7.5, 150 mM NaCl, 1 mM DTT). The adjusted protein solution was treated with purified His-TEV protease (ratio hSOS1:TEV, w/w, 30:1) for 16 h at 4° C. and afterwards passed over a Ni-NTA column to remove non-cleaved hSOS1 protein, cleaved tag and His-TEV. The pooled flow through fractions with the processed hSOS1 were concentrated using a Amicon Ultra 15 Ultracel-10 device (Centrifugal Filter 10000 NMWL; Merck-Millipore #UFC901024) and applied to size-exclusion chromatography column with Superdex 200 HR prep grade (GE Healthcare) in SEC buffer (25mM Tris HCl 7.5, 100mM NaCl). The final yield of tag-free protein for SOS1_12 was about 245 mg per liter cell culture was. The final product (tag-free) concentration for hSOS1_12 was 30.7 mg/mL.

Complex formation and Crystallization of hSOS1_12 with SOS1 inhibitors The catalytic domain of human SOS1 (hSOS1) in complex with inhibitors can be crystallized using construct hSOS1_12. It is identical to the construct published by Freedman et al. (Ref. 1). It comprises of hSOS1 residues Glu564 to Thr1049 with an additional four amino acids (Gly-Ala-Met-Ala) at the N-terminus and is shown in Figures X1 and X2 below. For inhibitor-complex formation, frozen aliquots of the hSOS1_12 protein (concentration 30.7 mg/ml) in buffer (25 mM Tris HCl 7.5/50 mM NaCl/1 mM DTT) are thawed and the respective SOS1 inhibitor is added before setting up of the crystallization experiment (co-crystallization approach) or soaked into pre-formed apo crystals (soaking approach). For the co-crystallization approach, the inhibitor is added from a 200 mM DMSO stock solution to a final inhibitor concentration of 2 mM and the mix is incubated over night at 4° C. The complex can be crystallized using the Hanging Drop method. Crystals grow at 20° C. Drops are made from 1 μl hSOS1_12:inhibitor mix, 1 μl reservoir solution (20-30% % (v/v) ethylenglycole) and 0.2 μl seed stock. The seed stock was generated from hSOS1 crystals previously obtained in an initial screen using the same hSOS1_12 construct and a reservoir solution of 25% ethylene glycol. For the soaking approach, apo SOS1 crystals (grown using the same procedure as described above, just without addition of an inhibitor) are soaked for 2 to 24 hours with 2 mM ligand.

Data Collection and Processing

SOS1-inhibitor crystals are directly shock frozen in liquid nitrogen. Diffraction data sets collected at synchrotrons can be processed using the programs XDS and XDSAPP.

Structure Determination and Refinement

The crystal form described here was first obtained and solved for a hSOS1_12 crystal grown in the presence of another inhibitor of the same chemical series, from a reservoir solution composed of 25% ethylene glycol. This initial structure was solved using the Molecular Replacement method with the program PHASER from the CCP4 program suite and the published structure of hSOS1 (PDB entry 2ii0, Ref. 1) as search model. The data sets for further SOS1:inhibitor crystal structures can be solved by Molecular Replacement using PHASER and an earlier in-house SOS1:inhibitor co-complex structure as starting model. 3D models for the inhibitors are generated using the program Discovery Studio (company Biovia) and parameter files for crystallographic refinement and model building are generated using software PRODRG. The inhibitors can be built manually built into the electron density maps using the program COOT, followed by several cycles of refinement (using program REFMAC as part of the CCP4 program suite) and rebuilding in COOT.

FIG. XI: Sequence of hSOS1_12 with
N-terminal His tag (HislO-hSOSl_12)
before cleavage by
TEV protease.
MGHHHHHHHH HHSSGHIEGR HMLETSLYKK
AGSDYDIPTT ENLYFQGAMA EEQMRLPSAD
VYRFAEPDSE ENIIFEENMQ PKAGIPIIKA
GTVIKLIERL TYHMYADPNF VRTFLTTYRS
FCKPQELLSL IIERFEIPEP EPTEADRIAI
ENGDQPLSAE LKRFRKEYIQ PVQLRVLNVC
RHWVEHHFYD FERDAYLLQR MEEFIGTVRG
KAMKKWVESI TKIIQRKKIA RDNGPGHNIT
FQSSPPTVEW HISRPGHIET FDLLTLHPIE
IARQLTLLES DLYRAVQPSE LVGSVWTKED
KEINSPNLLK MIRHTTNLTL WFEKCIVETE
NLEERVAVVS RIIEILQVFQ ELNNFNGVLE
VVSAMNSSPV YRLDHTFEQI PSRQKKILEE
AHELSEDHYK KYLAKLRSIN PPCVPFFGIY
LTNILKTEEG NPEVLKRHGK ELINFSKRRK
VAEITGEIQQ YQNQPYCLRV ESDIKRFFEN
LNPMGNSMEK EFTDYLFNKS LEIEPRNPKP
LPRFPKKYSY PLKSPGVRPS NPRPGT
FIG. X2: Sequence of hSOS1_12
after cleavage by TEV protease.
GAMAEEQMRL PSADVYRFAE PDSEENIIFE
ENMQPKAGIP IIKAGTVIKL IERLTYHMYA
DPNFVRTFLT TYRSFCKPQE LLSLIIERFE
IPEPEPTEAD RIAIENGDQP LSAELKRFRK
EYIQPVQLRV LNVCRHWVEH HFYDFERDAY
LLQRMEEFIG TVRGKAMKKW VESITKIIQR
KKIARDNGPG HNITFQSSPP TVEWHISRPG
HIETFDLLTL HPIEIARQLT LLESDLYRAV
QPSELVGSVW TKEDKEINSP NLLKMIRHTT
NLTLWFEKCI VETENLEERV AVVSRIIEIL
QVFQELNNFN GVLEVVSAMN SSPVYRLDHT
FEQIPSRQKK ILEEAHELSE DHYKKYLAKL
RSINPPCVPF FGIYLTNILK TEEGNPEVLK
RHGKELINFS KRRKVAEITG EIQQYQNQPY
CLRVESDIKR FFENLNPMGN SMEKEFTDYL
FNKSLEIEPR NPKPLPRFPK KYSYPLKSPG
VRPSNPRPGT

1. A compound of formula (Ia) 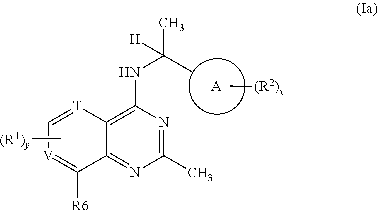
wherein
R¹ is selected from
—H, halogen, —OH, —CN, —NO₂, C₁-C₆-alkylsulfanyl,
—NR^{a}R^{b}, wherein R^{a} and R^{b} are independently selected from —H or C₁-C₆-alkyl,
C₁-C₆-alkyl, C₁-C₆-alkoxy, C₂-C₆-alkenyl, C₂-C₆-alkynyl, C₃-C₈-cycloalkyl,
C₄-C₃-cycloalkenyl, 4- to 7-membered heterocycloalkyl, 5- to 10 membered heterocycloalkenyl, heterospirocycloalkyl optionally substituted by an oxo-group (═O), fused heterocycloalkyl optionally substituted by an oxo-group (═O), bridged heterocycloalkyl optionally substituted by an oxo-group (═O), phenyl, heteroaryl, C₁-C₆-haloalkyl, —C(═O)OH, —C(═O)OR^{c}, wherein R^{c} stands for C₁-C₆-alkyl, C₃-C₆-alkenyl, C₃-C₆-alkynyl, C₃-C₈-cycloalkyl or C₄-C₈-cycloalkenyl,
—N═S(═O)(R^{d})R^{e}, wherein R^{d} and R^{e} are independently selected from C₁-C₆-alkyl, C₂-C₆-alkenyl, C₂-C₆-alkynyl, C₃-C₈-cycloalkyl or C₄-C₈-cycloalkenyl,
—NH—C(O)—C₁-C₆-alkyl,
—NH—C(O)—NR^{a}R^{b}, wherein R^{a} and R^{b} are selected independently from a hydrogen atom or a C₁-C₆-alkyl,
—NH—(CH₂)ₖ—NH—C(O)—C₁-C₆-alkyl, wherein k is 1 or 2,
—NH—(CH₂)_{I}—R^{f}, wherein I is 0, 1 or 2 and R^{f} stands for a 4- to 7-membered heterocycloalkyl, heteroaryl or C₁-C₆-alkylsulfonyl,
whereby in all foregoing definitions the C₁-C₆-alkyl-, C₁-C₆-alkoxy-, the 4- to 7-membered heterocycloalkyl and the heteroaryl can be optionally substituted, one or two or three times, identically or differently, with a halogen atom, hydroxy, oxo (═O), a cyano, nitro, C₁-C₆-alkyl, C₂-C₆-alkenyl, C₂-C₆-alkynyl, C₃-C₈-cycloalkyl, 4- to 7-membered heterocycloalkyl, C₁-C₆-alkoxy, C₁-C₆-haloalkyl, C₁-C₆-haloalkoxy, C₁-C₆-alkylsulfonyl, phenyl, benzyl, heteroaryl, —CH₂-heteroaryl, C₃-C₈-cycloalkoxy. phenyloxy, heteroaryloxy, —NH—C(O)—C₁-C₆-alkyl or —NR^{a}R^{b}, wherein R^{a} and R^{b} are independently selected from a hydrogen atom or C₁-C₆-alkyl,
—O—(CH₂)_{z}-phenyl, —O(CH₂)_{z}-C₄-C₇-heterocycloalkyl, —O(CH₂)_{z}-heteroaryl, wherein z is 0, 1 or 2, and the phenyl, heterocycloalkyl and heteroaryl can optionally be substituted with a group selected from hydroxy, heterocycloalkyl or heterocaclyoalkenyl, which both can be substituted with a methyl- and/or oxo-group, 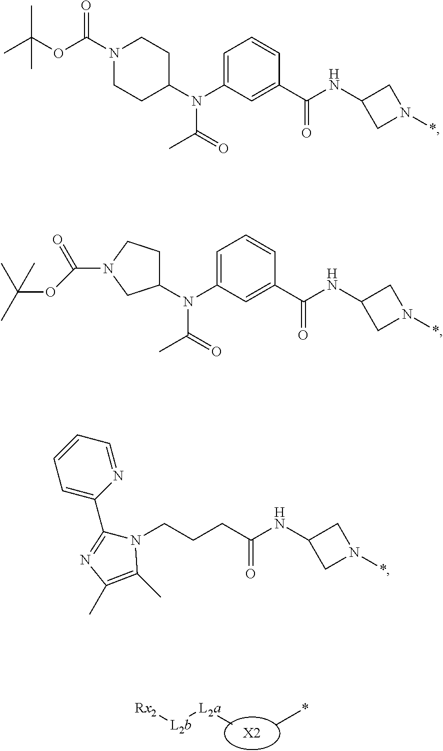
wherein L₂a stands for C(O), L₂b stands for a bond or C₁-C₆-alkylene, X2 stands for 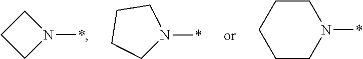
and Rx₂ stands for 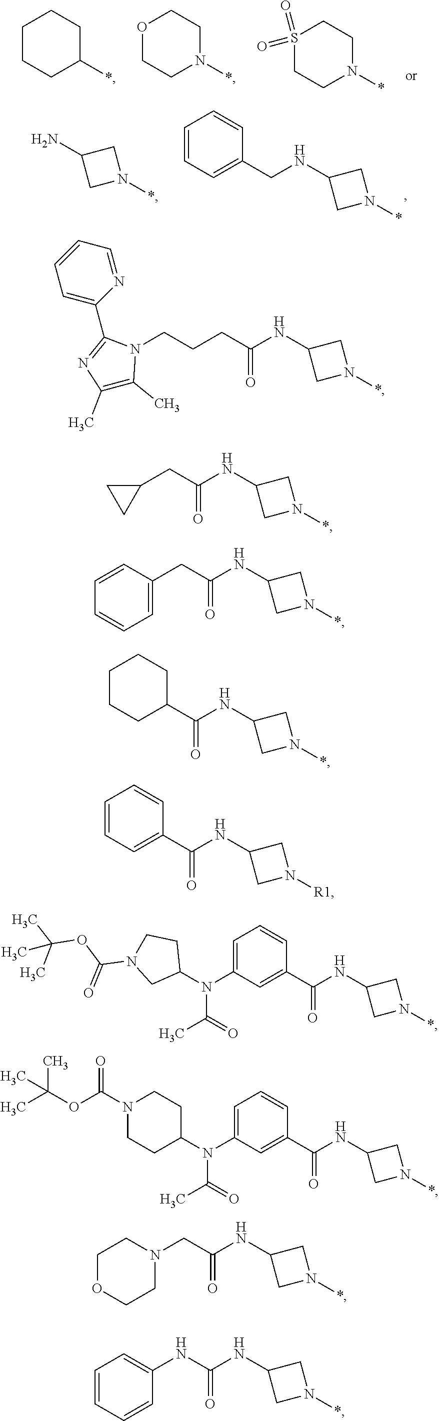 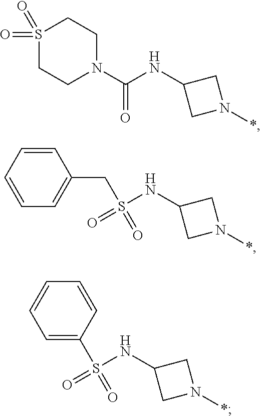
or in which a further R¹ as defined above can be directly attached to a first R¹ equaling C₁-C₆-alkyl, C₁-C₆-alkoxy, C₂-C₆-alkenyl, C₂-C₆-alkynyl, C₃-C₈-cycloalkyl, C₄-C₈-cycloalkenyl, 4- to 7-membered heterocycloalkyl, 5- to 10 membered heterocycloalkenyl, heterospirocycloalkyl, fused heterocycloalkyl, bridged heterocycloalkyl, phenyl, heteroaryl, C₁-C₆-haloalkyl,
y is 1, 2 or 3;
and either both T and V stand for nitrogen or T stands for carbon and V for nitrogen or T for nitrogen and V for carbon;
A is selected from the group consisting of C₆₋₁₀aryl, 5-10 membered heteroaryl and 9-10 membered bicyclic heterocyclyl;
R² is each independently selected from the group consisting of C₁₋₄alkyl, C₂₋₄alkenyl, C₂₋₄alkinyl, C₁₋₄haloalkyl, hydroxy-C₁₋₄alkyl, hydroxy-C₁₋₄haloalkyl, C₃₋₆cycloalkyl, 3-6 membered heterocyclyl, hydroxy-C₃₋₆cycloalkyl, C₁₋₄haloalkyl substituted with a 3-6 membered heterocyclyl, 3-6 membered heterocyclyl substituted with hydroxy, halogen, —NH₂, —SO₂—C₁₋₄alkyl and the bivalent substituent ═O, while ═O may only be a substituent in a non-aromatic ring;
R⁶ is selected from the group consisting of —H, halogen, C₁₋₄alkyl, C₃₋₇-cycloalkyl, C₄₋₇heterocycloalkyl optionally comprising 1 or 2 nitrogen, 1 oxygen or 1 sulphur atom, —O—C₁₋₄alkyl, —NH₂, —NH(C₁₋₄alkyl) or —NH(C₁₋₄alkyl)₂,
x is 1, 2 or 3;
or a tautomer, an N-oxide, a hydrate, a solvate, or a salt thereof, or a mixture thereof.

2. The compound according to claim 1, wherein
R¹ is selected from
—H, —Br, —OH, —NO₂, —CH₃, 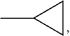
—O—CH₃, —O—CH₂—CH₃, —O—CH(CH₃)₂, —O—(CH₂)₃CH₃, —O—(CH₂)₂CH(CH₃)₂, 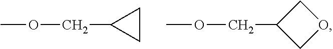
—O—CH₂-phenyl, —O—(CH₂)₂—O—CH₃, —O—(CH₂)₂—S(O)₂—CH₃, —CH₂—OH, —C(CH₃)₂—OH, —C(O)OH, —C(O)OCH₃, —NH₂, —NH(CH₃), —N(CH3)₂, 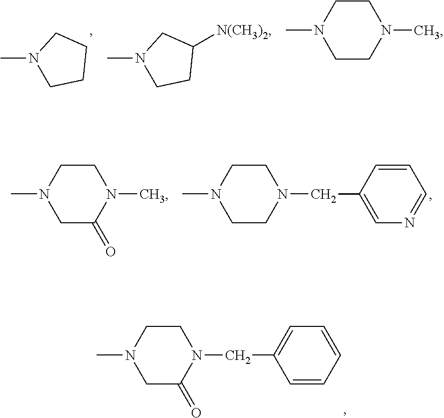
—NH—(CH₂)₂—NH—C(O)—CH₃, —NH—(CH₂)₂-morpholino, —NH—C(O)—CH₃, —NH—C(O)—NH—CH₃, —NH—C(O)—N(CH₃)₂, —NH—S(O)₂—CH₃, —N═S(O)(CH₃)₂, 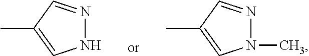
Y is 1 or 2;
A is selected from the group consisting of C₆₋₁₀aryl, 5-10 membered heteroaryl and 9-10 membered bicyclic heterocyclyl;
R² is each independently selected from the group consisting of C₁₋₄alkyl, C₂₋₄alkenyl, C₂₋₄alkinyl, C₁₋₄haloalkyl, hydroxy-C₁₋₄alkyl, hydroxy-C₁₋₄haloalkyl, C₃₋₆cycloalkyl, 3-6 membered heterocyclyl, hydroxy-C₃₋₆cycloalkyl, C₁₋₄haloalkyl substituted with a 3-6 membered heterocyclyl, 3-6 membered heterocyclyl substituted with hydroxy, halogen, —NH₂, —SO₂-C₁₋₄alkyl and the bivalent substituent ═O, while ═O may only be a substituent in a non-aromatic ring
x is 1, 2 or 3;
or a tautomer, an N-oxide, a hydrate, a solvate, or a salt thereof, or a mixture thereof.

3. The compound according to claim 1, wherein
A is selected from the group consisting of C₆₋₁₀-aryl, 5-10 membered heteroaryl and 9-10 membered bicyclic heterocyclyl;
R² is each independently selected from the group consisting of C₁₋₄-alkyl, C₂₋₄-alkenyl, C₂₋₄-alkinyl, C₁₋₄-haloalkyl, hydroxy-C₁₋₄-alkyl, hydroxy-C₁₋₄-haloalkyl, C₃₋₆-cycloalkyl, 3-6 membered heterocyclyl, hydroxy-C₃₋₆-cycloalkyl, C₁₋₄-haloalkyl substituted with a 3-6 membered heterocyclyl, 3-6 membered heterocyclyl substituted with hydroxy, halogen, —NH₂, —SO₂—C₁₋₄-alkyl and the bivalent substituent ═O, while ═O may only be a substituent in a non-aromatic ring
x is 1, 2 or 3
or a tautomer, an N-oxide, a hydrate, a solvate, or a salt thereof, or a mixture thereof.

4. The compound according to claim 1, wherein
A is selected from the group consisting of C₆₋₁₀aryl, 5-10 membered heteroaryl and 9-10 membered bicyclic heterocyclyl;
x is 1 or 2
R² is each independently selected from the group consisting of C₁₋₄-alkyl, C₂₋₄-alkinyl, C₁₋₄-haloalkyl, hydroxy-C₁₋₄-haloalkyl, C₁₋₄-haloalkyl substituted with a 3-6 membered heterocyclyl, halogen, and the bivalent substituent ═O, while ═O may only be a substituent in a non-aromatic ring
or a tautomer, an N-oxide, a hydrate, a solvate, or a salt thereof, or a mixture thereof.

5. The compound according to claim 1, wherein 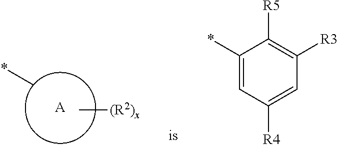
and wherein
R³ is selected from the group consisting of C₁₋₄-alkyl, C₁₋₄-haloalkyl, hydroxy-C₁₋₄-alkyl, hydroxy-C₁₋₄-haloalkyl, C₁₋₄-haloalkyl substituted with a 3-6 membered heterocyclyl, C₃₋₆-cycloalkyl, hydroxy-C₃₋₆-cycloalkyl, 3-6 membered heterocyclyl, 3-6 membered hydroxy-heterocyclyl, halogen and —SO₂—C₁₋₄-alkyl;
R⁴ is selected from the group consisting of hydrogen and —NH₂,
R⁵ is selected from the group consisting of hydrogen, C₁₋₄-alkyl and halogen;
or
R³ and R⁵ together with the carbon atoms they are attached form a 5-6 membered nonaromatic carbocycle, a 5-6 membered non-aromatic heterocycle or a 5-6 membered heteroaryl, wherein the 5-6 membered non-aromatic carbocycle, 5-6 membered nonaromatic heterocycle and 5-6 membered heteroaryl are all optionally substituted by one or more halogen or by an oxo group
or a tautomer, an N-oxide, a hydrate, a solvate, or a salt thereof, or a mixture thereof.

6. The compound according to claim 1, wherein
R³ is selected from the group consisting of C₁₋₄-haloalkyl, hydroxy-C₁₋₄-haloalkyl and C₁₋₄-haloalkyl substituted with a 3-6 membered heterocyclyl;
R⁴ is hydrogen;
R⁵ is selected from the group consisting of hydrogen, C₁₋₄-alkyl and fluorine;
or
R³ and R⁵ together with the carbon atoms they are attached form a 5-6 membered nonaromatic carbocycle, a 5-6 membered non-aromatic heterocycle or a 5-6 membered heteroaryl, wherein the 5-6 membered non-aromatic carbocycle, 5-6 membered nonaromatic heterocycle and 5-6 membered heteroaryl are all optionally substituted by one or more fluorine or by an oxo group
or a tautomer, an N-oxide, a hydrate, a solvate, or a salt thereof, or a mixture thereof.

7. The compound according to claim 1, wherein 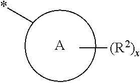
is selected from 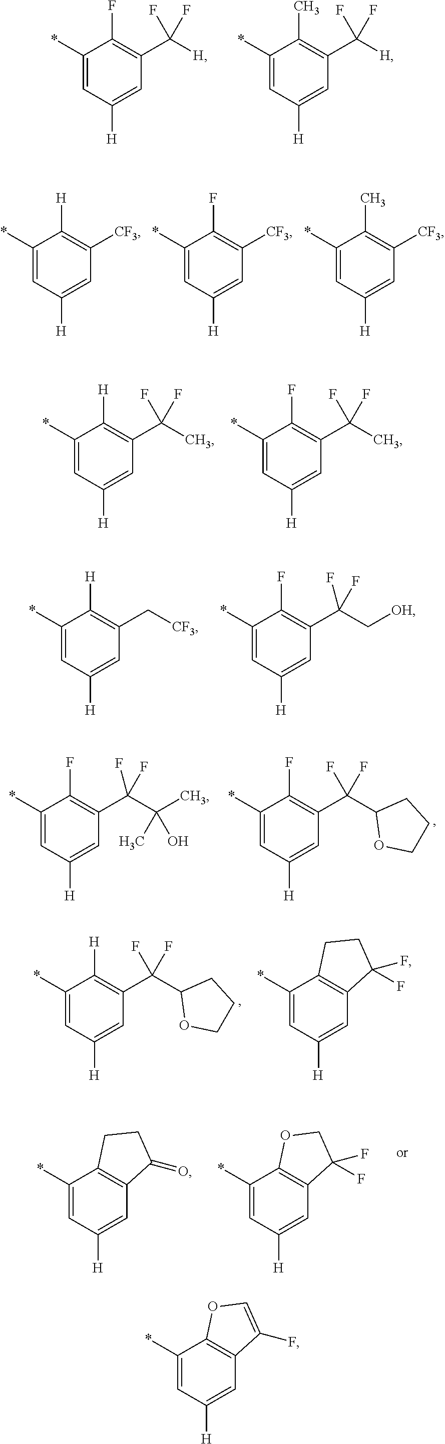
or a stereoisomer, a tautomer, an N-oxide, a hydrate, a solvate, or a salt thereof, or a mixture thereof.

8. The compound according to claim 1, wherein V is nitrogen and T is carbon or a stereoisomer, a tautomer, an N-oxide, a hydrate, a solvate, or a salt thereof, or a mixture thereof.

9. The compound according to claim 1, wherein y=1 and R¹ is selected from 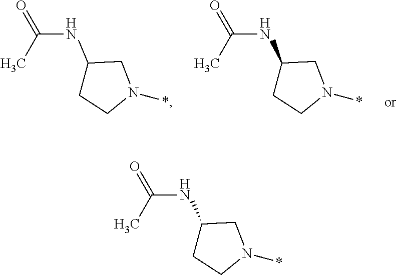
or a stereoisomer, a tautomer, an N-oxide, a hydrate, a solvate, or a salt thereof, or a mixture thereof.

10. The compound according to claim 1, wherein V is nitrogen, T is carbon, y=1,
R¹ is selected from 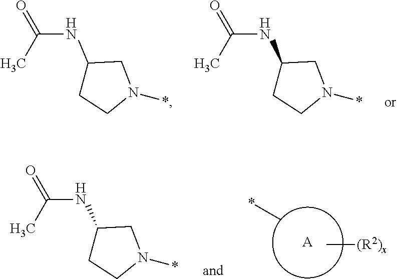
is selected from 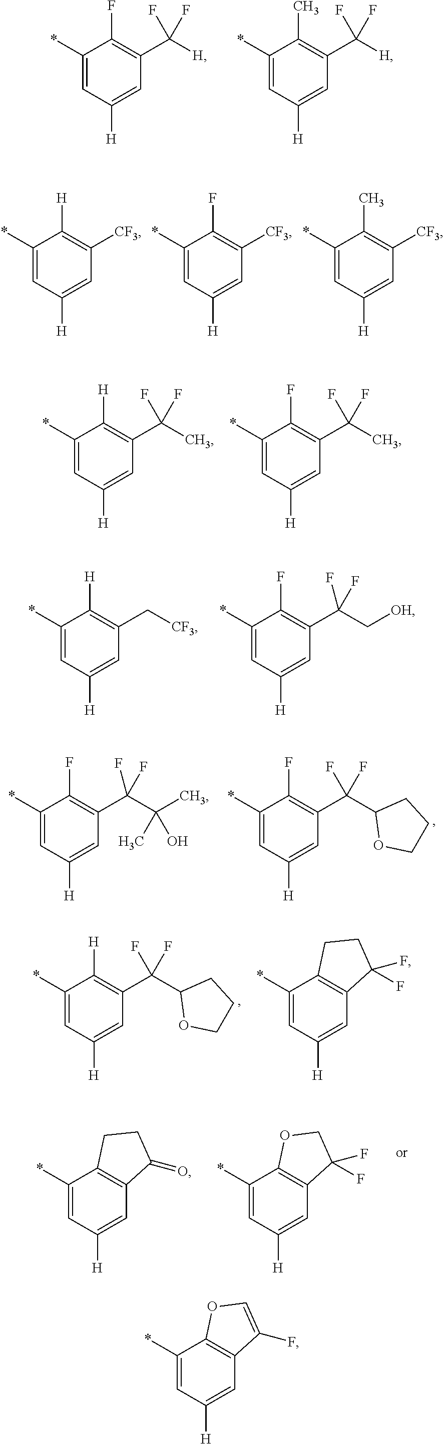
or a stereoisomer, a tautomer, an N-oxide, a hydrate, a solvate, or a salt thereof, or a mixture thereof.

11. The compound according to claim 1, which is selected from the group consisting of:
N-{(1R)-1-[3-(difluoromethyl)-2-fluorophenyl]ethyl}-6-ethoxy-2-methylpyrido[3,4-d]pyrimidin-4-amine
N-{(1R)-1-[3-(difluoromethyl)-2-fluorophenyl]ethyl}-6-fluoro-2-methylpyrido[3,4-d]pyrimidin-4-amine
N-[(3R)-1-[4-[[(1R)-1-[3-(difluoromethyl)-2-fluoro-phenyl]ethyl]amino]-2-methyl-pyrido[3,4-d]pyrimidin-6-yl]pyrrolidin-3-yl]acetamide
N-[(3S)-1-[4-[[(1R)-1-[3-(difluoromethyl)-2-fluoro-phenyl]ethyl]amino]-2-methyl-pyrido[3,4-d]pyrimidin-6-yl]pyrrolidin-3-yl]acetamide
N-[(1R)-1-[3-(difluoromethyl)-2-fluoro-phenyl]ethyl]-2-methyl-6-pyrrolidin-1-yl-pyrido[3,4-d]pyrimidin-4-amine
N-{(1R)-1-[3-(difluoromethyl)-2-methylphenyl]ethyl}-6-fluoro-2-methylpyrido[3,4-d]pyrimidin-4-amine
N-[(3R)-1-[4-[[(1R)-1-[3-(difluoromethyl)-2-methyl-phenyl]ethyl]amino]-2-methyl-pyrido[3,4-d]pyrimidin-6-yl]pyrrolidin-3-yl]acetamide
N-[(3S)-1-[4-[[(1R)-1-[3-(difluoromethyl)-2-methyl-phenyl]ethyl]amino]-2-methyl-pyrido[3,4-d]pyrimidin-6-yl]pyrrolidin-3-yl]acetamide
N-[(1R)-1-[3-(difluoromethyl)-2-methyl-phenyl]ethyl]-2-methyl-6-pyrrolidin-1-yl-pyrido[3,4-d]pyrimidin-4-amine
6-fluoro-2-methyl-N-[(1R)-1-[3-(trifluoromethyl)phenyl]ethyl]pyrido[3,4-d]pyrimidin-4-amine
N-[(3R)-1-[2-methyl-4-[[(1R)-1-[3-(trifluoromethyl)phenyl]ethyl]amino]pyrido[3,4-d]pyrimidin-6-yl]pyrrolidin-3-yl]acetamide
N-[(3S)-1-[2-methyl-4-[[(1R)-1-[3-(trifluoromethyl)phenyl]ethyl]amino]pyrido[3,4-d]pyrimidin-6-yl]pyrrolidin-3-yl]acetamide
N-[(1R)-1-[3-(1,1-difluoroethyl)phenyl]ethyl]-6-fluoro-2-methyl-pyrido[3,4-d]pyrimidin-4-amine
N-[(3R)-1-[4-[[(1R)-1-[3-(1,1-difluoroethyl)phenyl]ethyl]amino]-2-methyl-pyrido[3,4-d]pyrimidin-6-yl]pyrrolidin-3-yl]acetamide
N-[(3S)-1-[4-[[(1R)-1-[3-(1,1-difluoroethyl)phenyl]ethyl]amino]-2-methyl-pyrido[3,4-d]pyrimidin-6-yl]pyrrolidin-3-yl]acetamide
N-[(1R)-1-[3-(1,1-difluoroethyl)phenyl]ethyl]-6-fluoro-2-methyl-pyrido[3,4-d]pyrimidin-4-amine
N-[(3R)-1-[4-[[(1R)-1-[3-(1,1-difluoroethyl)-2-fluoro-phenyl]ethyl]amino]-2-methyl-pyrido[3,4-d]pyrimidin-6-yl]pyrrolidin-3-yl]acetamide
N-[(3S)-1-[4-[[(1R)-1-[3-(1,1-difluoroethyl)-2-fluoro-phenyl]ethyl]amino]-2-methyl-pyrido[3,4-d]pyrimidin-6-yl]pyrrolidin-3-yl]acetamide
N-{(1R)-1-[3-(difluoromethyl)-2-fluorophenyl]ethyl}-6-fluoro-2,8-dimethylpyrido[3,4-d]pyrimidin-4-amine
N-{(3R)-1-[4-({(1R)-1-[3-(difluoromethyl)-2-fluorophenyl]ethyl}amino)-2,8-dimethylpyrido[3,4-d]pyrimidin-6-yl]pyrrolidin-3-yl}acetamide
N-{(1R)-1-[3-(difluoromethyl)-2-fluorophenyl]ethyl}-6-[(3R)-3-(dimethylamino)pyrrolidin-1-yl]-2,8-dimethylpyrido[3,4-d]pyrimidin-4-amine
1-{4-[4-({(1R)-1-[3-(difluoromethyl)-2-fluorophenyl]ethyl}amino)-2,8-dimethylpyrido[3,4-d]pyrimidin-6-yl]piperazin-1-yl}ethan-1-one
N-{(1R)-1-[3-(difluoromethyl)-2-fluorophenyl]ethyl}-2,8-dimethyl-6-(4-methylpiperazin-1-yl)pyrido[3,4-d]pyrimidin-4-amine
2-[4-({(1R)-1-[3-(difluoromethyl)-2-fluorophenyl]ethyl}amino)-2,8-dimethylpyrido[3,4-d]pyrimidin-6-yl]-2,6-diazaspiro[3.4]octan-7-one
N-{(3S)-1-[4-({(1R)-1-[3-(difluoromethyl)phenyl]ethyl}amino)-2-methylpyrido[3,4-d]pyrimidin-6-yl]pyrrolidin-3-yl}acetamide
N-{(3S)-1-[2-methyl-4-({(1R)-1-[2-methyl-3-(trifluoromethyl)phenyl]ethyl}amino)pyrido[3,4-d]pyrimidin-6-yl]pyrrolidin-3-yl}acetamide
6-ethoxy-2-methyl-N-{(1R)-1-[3-(trifluoromethyl)phenyl]ethyl}pyrido[3,4-d]pyrimidin-4-amine
1-(3-{(1R)-1-[(6-ethoxy-2-methylpyrido[3,4-d]pyrimidin-4-yl)amino]ethyl}-2-fluorophenyl)-1,1-difluoro-2-methylpropan-2-ol
6-ethoxy-N-{(1R)-1-[2-fluoro-3-(trifluoromethyl)phenyl]ethyl}-2-methylpyrido[3,4-d]pyrimidin-4-amine
N-{(1R)-1-[3-(1,1-difluoroethyl)-2-fluorophenyl]ethyl}-6-ethoxy-2-methylpyrido[3,4-d]pyrimidin-4-amine
N-{(1R)-1-[3-(difluoromethyl)-2-methylphenyl]ethyl}-6-ethoxy-2-methylpyrido[3,4-d]pyrimidin-4-amine
6-ethoxy-2-methyl-N-{(1R)-1-[2-methyl-3-(trifluoromethyl)phenyl]ethyl}pyrido[3,4-d]pyrimidin-4-amine
N-{(1R)-1-[3-(difluoromethyl)phenyl]ethyl}-6-ethoxy-2-methylpyrido[3,4-d]pyrimidin-4-amine
N-{(1R)-1-[3-amino-5-(trifluoromethyl)phenyl]ethyl}-6-ethoxy-2-methylpyrido[3,4-d]pyrimidin-4-amine
6-methoxy-2-methyl-N-{(1R)-1-[3-(trifluoromethyl)phenyl]ethyl}pyrido[3,4-d]pyrimidin-4-amine
N-{(1R)-1-[3-(difluoromethyl)-2-fluorophenyl]ethyl}-6-methoxy-2-methylpyrido[3,4-d]pyrimidin-4-amine
N-{(1R)-1-[2-fluoro-3-(trifluoromethyl)phenyl]ethyl}-6-methoxy-2-methylpyrido[3,4-d]pyrimidin-4-amine
N-{(1R)-1-[3-(difluoromethyl)-2-methylphenyl]ethyl}-6-methoxy-2-methylpyrido[3,4-d]pyrimidin-4-amine
6-methoxy-2-methyl-N-{(1R)-1-[2-methyl-3-(trifluoromethyl)phenyl]ethyl}pyrido[3,4-d]pyrimidin-4-amine
N-{(1R)-1-[3-(1,1-difluoroethyl)-2-fluorophenyl]ethyl}-6-methoxy-2-methylpyrido[3,4-d]pyrimidin-4-amine
2,2-difluoro-2-(2-fluoro-3-{(1R)-1-[(6-methoxy-2-methylpyrido[3,4-d]pyrimidin-4-yl)amino]ethyl}phenyl)ethan-1-ol
1,1-difluoro-1-(2-fluoro-3-{(1R)-1-[(6-methoxy-2-methylpyrido[3,4-d]pyrimidin-4-yl)amino]ethyl}phenyl)-2-methylpropan-2-ol
N-{(3R)-1-[4-({(1R)-1-[3-(1,1-difluoro-2-hydroxyethyl)-2-fluorophenyl]ethyl}amino)-2-methylpyrido[3,4-d]pyrimidin-6-yl]pyrrolidin-3-yl}acetamide
N-{(3R)-1-[4-({(1R)-1-[3-(1,1-difluoro-2-hydroxy-2-methylpropyl)-2-fluorophenyl]ethyl}amino)-2-methylpyrido[3,4-d]pyrimidin-6-yl]pyrrolidin-3-yl}acetamide
N-{(3R)-1-[4-({(1R)-1-[2-fluoro-3-(trifluoromethyl)phenyl]ethyl}amino)-2-methylpyrido[3,4-d]pyrimidin-6-yl]pyrrolidin-3-yl}acetamide
N-{(3R)-1-[2-methyl-4-({(1R)-1-[2-methyl-3-(trifluoromethyl)phenyl]ethyl}amino)pyrido[3,4-d]pyrimidin-6-yl]pyrrolidin-3-yl}acetamide
N-{(3R)-1-[4-({(1R)-1-[3-(difluoromethyl)phenyl]ethyl}amino)-2-methylpyrido[3,4-d]pyrimidin-6-yl]pyrrolidin-3-yl}acetamide
N-[(3R)-1-(2-methyl-4-{[(1R)-1-(2-methylphenyl)ethyl]amino}pyrido[3,4-d]pyrimidin-6-yl)pyrrolidin-3-yl]acetamide
N-[(3R)-1-(2-methyl-4-{[(1R)-1-(3-methylphenyl)ethyl]amino}pyrido[3,4-d]pyrimidin-6-yl)pyrrolidin-3-yl]acetamide
N-[(3R)-1-(2-methyl-4-{[(1R)-1-(4-methylphenyl)ethyl]amino}pyrido[3,4-d]pyrimidin-6-yl)pyrrolidin-3-yl]acetamide
N-[(3R)-1-(4-{[(1R)-1-(2-fluorophenyl)ethyl]amino}-2-methylpyrido[3,4-d]pyrimidin-6-yl)pyrrolidin-3-yl]acetamide
N-[(3R)-1-(4-{[(1R)-1-(3-fluorophenyl)ethyl]amino}-2-methylpyrido[3,4-d]pyrimidin-6-yl)pyrrolidin-3-yl]acetamide
N-[(3R)-1-(4-{[(1R)-1-(4-fluorophenyl)ethyl]amino}-2-methylpyrido[3,4-d]pyrimidin-6-yl)pyrrolidin-3-yl]acetamide
N-[(3R)-1-(4-{[(1R)-1-(2-methoxyphenyl)ethyl]amino}-2-methylpyrido[3,4-d]pyrimidin-6-yl)pyrrolidin-3-yl]acetamide
N-[(3R)-1-(4-{[(1R)-1-(3-methoxyphenyl)ethyl]amino}-2-methylpyrido[3,4-d]pyrimidin-6-yl)pyrrolidin-3-yl]acetamide
N-[(3R)-1-(4-{[(1R)-1-(2-chlorophenyl)ethyl]amino}-2-methylpyrido[3,4-d]pyrimidin-6-yl)pyrrolidin-3-yl]acetamide
N-[(3R)-1-(4-{[(1R)-1-(3-chlorophenyl)ethyl]amino}-2-methylpyrido[3,4-d]pyrimidin-6-yl)pyrrolidin-3-yl]acetamide
N-{(3R)-1-[4-({(1RS)-1-[2-(difluoromethyl)phenyl]ethyl}amino)-2-methylpyrido[3,4-d]pyrimidin-6-yl]pyrrolidin-3-yl}acetamide
N-{(3R)-1-[4-({(1RS)-1-[2-(difluoromethoxy)phenyl]ethyl}amino)-2-methylpyrido[3,4-d]pyrimidin-6-yl]pyrrolidin-3-yl}acetamide
N-{(3R)-1-[4-({(1R)-1-[3-(difluoromethoxy)phenyl]ethyl}amino)-2-methylpyrido[3,4-d]pyrimidin-6-yl]pyrrolidin-3-yl}acetamide
N-{(3R)-1-[2-methyl-4-({(1R)-1-[3-(trifluoromethoxy)phenyl]ethyl}amino)pyrido[3,4-d]pyrimidin-6-yl]pyrrolidin-3-yl}acetamide
N-[(3R)-1-(4-{[(1R)-1-(3-bromophenyl)ethyl]amino}-2-methylpyrido[3,4-d]pyrimidin-6-yl)pyrrolidin-3-yl]acetamide
N-[(3R)-1-(2-methyl-4-{[(1R)-1-{3-[(trifluoromethyl)sulfanyl]phenyl}ethyl]amino}pyrido[3,4-d]pyrimidin-6-yl)pyrrolidin-3-yl]acetamide
N-{(3R)-1-[2-methyl-4-({(1R)-1-[3-(pentafluoro-lambda⁶-sulfanyl)phenyl]ethyl}amino)pyrido[3,4-d]pyrimidin-6-yl]pyrrolidin-3-yl}acetamide
methyl 3-[(1R)-1-({6-[(3R)-3-acetamidopyrrolidin-1-yl]-2-methylpyrido[3,4-d]pyrimidin-4-yl}amino)ethyl]benzoate
N-[(3R)-1-(4-{[(1R)-1-(3-cyanophenyl)ethyl]amino}-2-methylpyrido[3,4-d]pyrimidin-6-yl)pyrrolidin-3-yl]acetamide
N-[(3R)-1-(2-methyl-4-{[(1R)-1-(3-nitrophenyl)ethyl]amino}pyrido[3,4-d]pyrimidin-6-yl)pyrrolidin-3-yl]acetamide
tert-butyl {3-[(1RS)-1-({6-[(3R)-3-acetamidopyrrolidin-1-yl]-2-methylpyrido[3,4-d]pyrimidin-4-yl}amino)ethyl]phenyl}carbamate
N-[(3R)-1-(4-{[(1R)-1-(4-fluoro-3-methylphenyl)ethyl]amino}-2-methylpyrido[3,4-d]pyrimidin-6-yl)pyrrolidin-3-yl]acetamide
N-[(3R)-1-(4-{[(1R)-1-(2,3-difluorophenyl)ethyl]amino}-2-methylpyrido[3,4-d]pyrimidin-6-yl)pyrrolidin-3-yl]acetamide
N-[(3R)-1-(4-{[(1R)-1-(3,4-difluorophenyl)ethyl]amino}-2-methylpyrido[3,4-d]pyrimidin-6-yl)pyrrolidin-3-yl]acetamide
N-[(3R)-1-(4-{[(1R)-1-(2,4-difluorophenyl)ethyl]amino}-2-methylpyrido[3,4-d]pyrimidin-6-yl)pyrrolidin-3-yl]acetamide
N-[(3R)-1-(4-{[(1RS)-1-(3,5-difluorophenyl)ethyl]amino}-2-methylpyrido[3,4-d]pyrimidin-6-yl)pyrrolidin-3-yl]acetamide
N-[(3R)-1-(4-{[(1RS)-1-(2,6-difluorophenyl)ethyl]amino}-2-methylpyrido[3,4-d]pyrimidin-6-yl)pyrrolidin-3-yl]acetamide
N-[(3R)-1-(4-{[(1RS)-1-(2,5-difluorophenyl)ethyl]amino}-2-methylpyrido[3,4-d]pyrimidin-6-yl)pyrrolidin-3-yl]acetamide
N-[(3R)-1-(4-{[(1R)-1-(5-bromo-2-methylphenyl)ethyl]amino}-2-methylpyrido[3,4-d]pyrimidin-6-yl)pyrrolidin-3-yl]acetamide
N-[(3R)-1-(4-{[(1R)-1-(3-bromo-5-fluorophenyl)ethyl]amino}-2-methylpyrido[3,4-d]pyrimidin-6-yl)pyrrolidin-3-yl]acetamide
N-[(3R)-1-(4-{[(1R)-1-(3-bromo-4-fluorophenyl)ethyl]amino}-2-methylpyrido[3,4-d]pyrimidin-6-yl)pyrrolidin-3-yl]acetamide
N-[(3R)-1-(4-{[(1R)-1-(3-bromo-2-fluorophenyl)ethyl]amino}-2-methylpyrido[3,4-d]pyrimidin-6-yl)pyrrolidin-3-yl]acetamide
N-[(3R)-1-(4-{[(1R)-1-(5-bromo-2-fluorophenyl)ethyl]amino}-2-methylpyrido[3,4-d]pyrimidin-6-yl)pyrrolidin-3-yl]acetamide
N-[(3R)-1-(4-{[(1R)-1-(5-bromo-2-methoxyphenyl)ethyl]amino}-2-methylpyrido[3,4-d]pyrimidin-6-yl)pyrrolidin-3-yl]acetamide
N-[(3R)-1-(4-{[(1R)-1-(3-fluoro-1-benzofuran-7-yl)ethyl]amino}-2-methylpyrido[3,4-d]pyrimidin-6-yl)pyrrolidin-3-yl]acetamide.
N-[(3R)-1-(4-{[(1S)-1-(3-fluoro-1-benzofuran-7-yl)ethyl]amino}-2-methylpyrido[3,4-d]pyrimidin-6-yl)pyrrolidin-3-yl]acetamide
N-{(3R)-1-[2-methyl-4-({(1RS)-1-[2-(1H-pyrazol-1-yl)phenyl]ethyl}amino)pyrido[3,4-d]pyrimidin-6-yl]pyrrolidin-3-yl}acetamide
N-{(3R)-1-[4-({(1RS)-1-[3-(difluoromethyl)-1-methyl-1H-pyrazol-4-yl]ethyl}amino)-2-methylpyrido[3,4-d]pyrimidin-6-yl]pyrrolidin-3-yl}acetamide
N-{(3R)-1-[2-methyl-4-({(1RS)-1-[1-methyl-3-(trifluoromethyl)-1H-pyrazol-4-yl]ethyl}amino)pyrido[3,4-d]pyrimidin-6-yl]pyrrolidin-3-yl}acetamide
N-[(3R)-1-(4-{[(1RS)-1-(5-chloro-1,3-thiazol-2-yl)ethyl]amino}-2-methylpyrido[3,4-d]pyrimidin-6-yl)pyrrolidin-3-yl]acetamide
N-{(3R)-1-[2-methyl-4-({(1RS)-1-[3-(trifluoromethyl)-1,2,4-oxadiazol-5-yl]ethyl}amino)pyrido[3,4-d]pyrimidin-6-yl]pyrrolidin-3-yl}acetamide
N-[(3R)-1-(4-{[(1R)-1-(5-bromopyridin-3-yl)ethyl]amino}-2-methylpyrido[3,4-d]pyrimidin-6-yl)pyrrolidin-3-yl]acetamide
N-[(3R)-1-(4-{[(1R)-1-(6-aminopyridin-2-yl)ethyl]amino}-2-methylpyrido[3,4-d]pyrimidin-6-yl)pyrrolidin-3-yl]acetamide
N-{(3R)-1-[4-({(1R)-1-[3-amino-5-(trifluoromethyl)phenyl]ethyl}amino)-2-methylpyrido[3,4-d]pyrimidin-6-yl]pyrrolidin-3-yl}acetamide
N-[(3R)-1-(4-{[1-(3-aminophenyl)ethyl]amino}-2-methylpyrido[3,4-d]pyrimidin-6-yl)pyrrolidin-3-yl]acetamide (mixture of stereoisomers)
tert-butyl {3-[(1S)-1-({6-[(3R)-3-acetamidopyrrolidin-1-yl]-2-methylpyrido[3,4-d]pyrimidin-4-yl}amino)ethyl]phenyl}carbamate
tert-butyl {3-[(1R)-1-({6-[(3R)-3-acetamidopyrrolidin-1-yl]-2-methylpyrido[3,4-d]pyrimidin-4-yl}amino)ethyl]phenyl}carbamate
N-[(3R)-1-(4-{[(1S)-1-(3-aminophenyl)ethyl]amino}-2-methylpyrido[3,4-d]pyrimidin-6-yl)pyrrolidin-3-yl]acetamide
N-[(3R)-1-(4-{[(1R)-1-(3-aminophenyl)ethyl]amino}-2-methylpyrido[3,4-d]pyrimidin-6-yl)pyrrolidin-3-yl]acetamide
N-[(3R)-1-(4-{[(1R)-1-(3,5-difluorophenyl)ethyl]amino}-2-methylpyrido[3,4-d]pyrimidin-6-yl)pyrrolidin-3-yl]acetamide
N-[(3R)-1-(4-{[(1S)-1-(3,5-difluorophenyl)ethyl]amino}-2-methylpyrido[3,4-d]pyrimidin-6-yl)pyrrolidin-3-yl]acetamide
N-[(3R)-1-(4-{[(1S)-1-(2,6-difluorophenyl)ethyl]amino}-2-methylpyrido[3,4-d]pyrimidin-6-yl)pyrrolidin-3-yl]acetamide
N-[(3R)-1-(4-{[(1R)-1-(2,6-difluorophenyl)ethyl]amino}-2-methylpyrido[3,4-d]pyrimidin-6-yl)pyrrolidin-3-yl]acetamide
N-[(3R)-1-(4-{[(1R)-1-(2,5-difluorophenyl)ethyl]amino}-2-methylpyrido[3,4-d]pyrimidin-6-yl)pyrrolidin-3-yl]acetamide
N-[(3R)-1-(4-{[(1S)-1-(2,5-difluorophenyl)ethyl]amino}-2-methylpyrido[3,4-d]pyrimidin-6-yl)pyrrolidin-3-yl]acetamide
3-[(1R)-1-({6-[(3R)-3-acetamidopyrrolidin-1-yl]-2-methylpyrido[3,4-d]pyrimidin-4-yl}amino)ethyl]benzoic acid
N-{(3R)-1-[4-({(1R)-1-[3-(hydroxymethyl)phenyl]ethyl}amino)-2-methylpyrido[3,4-d]pyrimidin-6-yl]pyrrolidin-3-yl}acetamide
N-[(3R)-1-(4-{[(1R)-1-(3-hydroxyphenyl)ethyl]amino}-2-methylpyrido[3,4-d]pyrimidin-6-yl)pyrrolidin-3-yl]acetamide
N-{(3R)-1-[4-({(1R)-1-[3-(2,2-difluoroethoxy)phenyl]ethyl}amino)-2-methylpyrido[3,4-d]pyrimidin-6-yl]pyrrolidin-3-yl}acetamide
N-[(3R)-1-(4-{[(1R)-1-{3-[(E)-2-ethoxyethenyl]phenyl}ethyl]amino}-2-methylpyrido[3,4-d]pyrimidin-6-yl)pyrrolidin-3-yl]acetamide
N-{(1R)-1-[3-(difluoromethyl)phenyl]ethyl}-2-methyl-6-(4-methylpiperazin-1-yl)pyrido[3,4-d]pyrimidin-4-amine
N-{(1R)-1-[3-(difluoromethyl)-2-methylphenyl]ethyl}-2-methyl-6-(4-methylpiperazin-1-yl)pyrido[3,4-d]pyrimidin-4-amine
N-{(1R)-1-[3-(1,1-difluoroethyl)phenyl]ethyl}-2-methyl-6-(4-methylpiperazin-1-yl)pyrido[3,4-d]pyrimidin-4-amine
N-{(1R)-1-[3-(1,1-difluoroethyl)-2-fluorophenyl]ethyl}-2-methyl-6-(4-methylpiperazin-1-yl)pyrido[3,4-d]pyrimidin-4-amine
N-{(1R)-1-[2-fluoro-3-(trifluoromethyl)phenyl]ethyl}-2-methyl-6-(4-methylpiperazin-1-yl)pyrido[3,4-d]pyrimidin-4-amine
2,2-difluoro-2-{2-fluoro-3-[(1R)-1-{[2-methyl-6-(4-methylpiperazin-1-yl)pyrido[3,4-d]pyrimidin-4-yl]amino}ethyl]phenyl}ethan-1-ol
1,1-difluoro-1-{2-fluoro-3-[(1R)-1-{[2-methyl-6-(4-methylpiperazin-1-yl)pyrido[3,4-d]pyrimidin-4-yl]amino}ethyl]phenyl}-2-methylpropan-2-ol
N-{(1R)-1-[3-amino-5-(trifluoromethyl)phenyl]ethyl}-2-methyl-6-(4-methylpiperazin-1-yl)pyrido[3,4-d]pyrimidin-4-amine
N-{(1R)-1-[3-(difluoromethyl)phenyl]ethyl}-6-[(3R)-3-(dimethylamino)pyrrolidin-1-yl]-2-methylpyrido[3,4-d]pyrimidin-4-amine
N-{(1R)-1-[3-(1,1-difluoroethyl)phenyl]ethyl}-6-[(3R)-3-(dimethylamino)pyrrolidin-1-yl]-2-methylpyrido[3,4-d]pyrimidin-4-amine
N-{(1R)-1-[3-(1,1-difluoroethyl)-2-fluorophenyl]ethyl}-6-[(3R)-3-(dimethylamino)pyrrolidin-1-yl]-2-methylpyrido[3,4-d]pyrimidin-4-amine
6-[(3R)-3-(dimethylamino)pyrrolidin-1-yl]-N-{(1R)-1-[2-fluoro-3-(trifluoromethyl)phenyl]ethyl}-2-methylpyrido[3,4-d]pyrimidin-4-amine
N-{(1R)-1-[3-(difluoromethyl)-2-methylphenyl]ethyl}-6-[(3R)-3-(dimethylamino)pyrrolidin-1-yl]-2-methylpyrido[3,4-d]pyrimidin-4-amine
2-{3-[(1R)-1-({6-[(3R)-3-(dimethylamino)pyrrolidin-1-yl]-2-methylpyrido[3,4-d]pyrimidin-4-yl}amino)ethyl]-2-fluorophenyl}-2,2-difluoroethan-1-ol
1-{3-[(1R)-1-({6-[(3R)-3-(dimethylamino)pyrrolidin-1-yl]-2-methylpyrido[3,4-d]pyrimidin-4-yl}amino)ethyl]-2-fluorophenyl}-1,1-difluoro-2-methylpropan-2-ol
2-[4-({(1R)-1-[3-(difluoromethyl)phenyl]ethyl}amino)-2-methylpyrido[3,4-d]pyrimidin-6-yl]-2,6-diazaspiro[3.4]octan-7-one
2-[4-({(1R)-1-[3-(difluoromethyl)-2-methylphenyl]ethyl}amino)-2-methylpyrido[3,4-d]pyrimidin-6-yl]-2,6-diazaspiro[3.4]octan-7-one
2-[4-({(1R)-1-[3-(1,1-difluoroethyl)phenyl]ethyl}amino)-2-methylpyrido[3,4-d]pyrimidin-6-yl]-2,6-diazaspiro[3.4]octan-7-one
2-[4-({(1R)-1-[3-(1,1-difluoroethyl)-2-fluorophenyl]ethyl}amino)-2-methylpyrido[3,4-d]pyrimidin-6-yl]-2,6-diazaspiro[3.4]octan-7-one
2-[4-({(1R)-1-[2-fluoro-3-(trifluoromethyl)phenyl]ethyl}amino)-2-methylpyrido[3,4-d]pyrimidin-6-yl]-2,6-diazaspiro[3.4]octan-7-one
2-[4-({(1R)-1-[3-(1,1-difluoro-2-hydroxyethyl)-2-fluorophenyl]ethyl}amino)-2-methylpyrido[3,4-d]pyrimidin-6-yl]-2,6-diazaspiro[3.4]octan-7-one
2-[4-({(1R)-1-[3-amino-5-(trifluoromethyl)phenyl]ethyl}amino)-2-methylpyrido[3,4-d]pyrimidin-6-yl]-2,6-diazaspiro[3.4]octan-7-one
1-{4-[4-({(1R)-1-[3-(difluoromethyl)phenyl]ethyl}amino)-2-methylpyrido[3,4-d]pyrimidin-6-yl]piperazin-1-yl}ethan-1-one
1-{4-[4-({(1R)-1-[3-(1,1-difluoroethyl)phenyl]ethyl}amino)-2-methylpyrido[3,4-d]pyrimidin-6-yl]piperazin-1-yl}ethan-1-one
1-{4-[4-({(1R)-1-[3-(1,1-difluoroethyl)-2-fluorophenyl]ethyl}amino)-2-methylpyrido[3,4-d]pyrimidin-6-yl]piperazin-1-yl}ethan-1-one
1-{4-[4-({(1R)-1-[3-(difluoromethyl)-2-methylphenyl]ethyl}amino)-2-methylpyrido[3,4-d]pyrimidin-6-yl]piperazin-1-yl}ethan-1-one
1-{4-[4-({(1R)-1-[2-fluoro-3-(trifluoromethyl)phenyl]ethyl}amino)-2-methylpyrido[3,4-d]pyrimidin-6-yl]piperazin-1-yl}ethan-1-one
1-{4-[4-({(1R)-1-[3-(1,1-difluoro-2-hydroxy-2-methylpropyl)-2-fluorophenyl]ethyl}amino)-2-methylpyrido[3,4-d]pyrimidin-6-yl]piperazin-1-yl}ethan-1-one
1-{4-[4-({(1R)-1-[3-amino-5-(trifluoromethyl)phenyl]ethyl}amino)-2-methylpyrido[3,4-d]pyrimidin-6-yl]piperazin-1-yl}ethan-1-one
N⁴-{(1R)-1-[3-(difluoromethyl)-2-fluorophenyl]ethyl}-N⁶-ethyl-2-methylpyrido[3,4-d]pyrimidine-4,6-diamine
N⁶-cyclopropyl-N⁴-{(1R)-1-[3-(difluoromethyl)-2-fluorophenyl]ethyl}-2-methylpyrido[3,4-d]pyrimidine-4,6-diamine
N⁴-{(1R)-1-[3-(difluoromethyl)-2-fluorophenyl]ethyl}-2-methyl-N⁶-(propan-2-yl)pyrido[3,4-d]pyrimidine-4,6-diamine
N⁴-{(1R)-1-[3-(difluoromethyl)-2-fluorophenyl]ethyl}-N⁶-ethyl-N⁶,2-dimethylpyrido[3,4-d]pyrimidine-4,6-diamine
N⁴-{(1R)-1-[3-(difluoromethyl)-2-fluorophenyl]ethyl}-N⁶,2-dimethyl-N⁶-(prop-2-en-1-yl)pyrido[3,4-d]pyrimidine-4,6-diamine
N⁶-cyclopropyl-N⁴-{(1R)-1-[3-(difluoromethyl)-2-fluorophenyl]ethyl}-N⁶,2-dimethylpyrido[3,4-d]pyrimidine-4,6-diamine
N⁶-cyclobutyl-N⁴-{(1R)-1-[3-(difluoromethyl)-2-fluorophenyl]ethyl}-2-methylpyrido[3,4-d]pyrimidine-4,6-diamine
N⁴-{(1R)-1-[3-(difluoromethyl)-2-fluorophenyl]ethyl}-N⁶,2-dimethyl-N⁶-(propan-2-yl)pyrido[3,4-d]pyrimidine-4,6-diamine
N⁴-{(1R)-1-[3-(difluoromethyl)-2-fluorophenyl]ethyl}-N⁶-(2-methoxyethyl)-2-methylpyrido[3,4-d]pyrimidine-4,6-diamine
N-{(1R)-1-[3-(difluoromethyl)-2-fluorophenyl]ethyl}-2-methyl-6-(piperidin-1-yl)pyrido[3,4-d]pyrimidin-4-amine
N⁶-cyclopentyl-N⁴-{(1R)-1-[3-(difluoromethyl)-2-fluorophenyl]ethyl}-2-methylpyrido[3,4-d]pyrimidine-4,6-diamine
N-{(1R)-1-[3-(difluoromethyl)-2-fluorophenyl]ethyl}-2-methyl-6-(piperazin-1-yl)pyrido[3,4-d]pyrimidin-4-amine
(3S)-1-[4-({(1R)-1-[3-(difluoromethyl)-2-fluorophenyl]ethyl}amino)-2-methylpyrido[3,4-d]pyrimidin-6-yl]pyrrolidin-3-ol
(3R)-1-[4-({(1R)-1-[3-(difluoromethyl)-2-fluorophenyl]ethyl}amino)-2-methylpyrido[3,4-d]pyrimidin-6-yl]pyrrolidin-3-ol
N-{(1R)-1-[3-(difluoromethyl)-2-fluorophenyl]ethyl}-2-methyl-6-(morpholin-4-yl)pyrido[3,4-d]pyrimidin-4-amine
N⁴-{(1R)-1-[3-(difluoromethyl)-2-fluorophenyl]ethyl}-2-methyl-N⁶-{[(2RS)-oxetan-2-yl]methyl}pyrido[3,4-d]pyrimidine-4,6-diamine
N⁴-{(1R)-1-[3-(difluoromethyl)-2-fluorophenyl]ethyl}-2-methyl-N⁶-[(3R)-oxolan-3-yl]pyrido[3,4-d]pyrimidine-4,6-diamine
N⁴-{(1R)-1-[3-(difluoromethyl)-2-fluorophenyl]ethyl}-N⁶-(2-methoxyethyl)-N⁶,2-dimethylpyrido[3,4-d]pyrimidine-4,6-diamine
N⁴-{(1R)-1-[3-(difluoromethyl)-2-fluorophenyl]ethyl}-2-methyl-N⁶,N⁶-di(prop-2-en-1-yl)pyrido[3,4-d]pyrimidine-4,6-diamine
6-[2-azabicyclo[2.2.1]heptan-2-yl]-N-{(1R)-1-[3-(difluoromethyl)-2-fluorophenyl]ethyl}-2-methylpyrido[3,4-d]pyrimidin-4-amine (mixture of stereoisomers)
N-{(1R)-1-[3-(difluoromethyl)-2-fluorophenyl]ethyl}-2-methyl-6-(1-oxa-6-azaspiro[3.3]heptan-6-yl)pyrido[3,4-d]pyrimidin-4-amine
N-{(1R)-1-[3-(difluoromethyl)-2-fluorophenyl]ethyl}-2-methyl-6-(2-oxa-6-azaspiro[3.3]heptan-6-yl)pyrido[3,4-d]pyrimidin-4-amine
N⁶-cyclohexyl-N⁴-{(1R)-1-[3-(difluoromethyl)-2-fluorophenyl]ethyl}-2-methylpyrido[3,4-d]pyrimidine-4,6-diamine
4-{[4-({(1R)-1-[3-(difluoromethyl)-2-fluorophenyl]ethyl}amino)-2-methylpyrido[3,4-d]pyrimidin-6-yl]amino}pyrrolidin-2-one (mixture of stereoisomers)
4-[4-({(1R)-1-[3-(difluoromethyl)-2-fluorophenyl]ethyl}amino)-2-methylpyrido[3,4-d]pyrimidin-6-yl]piperazin-2-one
6-(1,4-diazepan-1-yl)-N-{(1R)-1-[3-(difluoromethyl)-2-fluorophenyl]ethyl}-2-methylpyrido[3,4-d]pyrimidin-4-amine
N-{(1R)-1-[3-(difluoromethyl)-2-fluorophenyl]ethyl}-2-methyl-6-(4-methylpiperazin-1-yl)pyrido[3,4-d]pyrimidin-4-amine
N-{(1R)-1-[3-(difluoromethyl)-2-fluorophenyl]ethyl}-2-methyl-6-[(3R)-3-methylmorpholin-4-yl]pyrido[3,4-d]pyrimidin-4-amine
N-{(1R)-1-[3-(difluoromethyl)-2-fluorophenyl]ethyl}-2-methyl-6-[(3S)-3-methylmorpholin-4-yl]pyrido[3,4-d]pyrimidin-4-amine
(3R)-1-[4-({(1R)-1-[3-(difluoromethyl)-2-fluorophenyl]ethyl}amino)-2-methylpyrido[3,4-d]pyrimidin-6-yl]piperidin-3-ol
(3S)-1-[4-({(1R)-1-[3-(difluoromethyl)-2-fluorophenyl]ethyl}amino)-2-methylpyrido[3,4-d]pyrimidin-6-yl]piperidin-3-ol
N⁴-{(1R)-1-[3-(difluoromethyl)-2-fluorophenyl]ethyl}-2-methyl-N⁶-(oxan-4-yl)pyrido[3,4-d]pyrimidine-4,6-diamine
N⁴-{(1R)-1-[3-(difluoromethyl)-2-fluorophenyl]ethyl}-2-methyl-N⁶-{[(2R)-oxolan-2-yl]methyl}pyrido[3,4-d]pyrimidine-4,6-diamine
N-{(1R)-1-[3-(difluoromethyl)-2-fluorophenyl]ethyl}-6-[(3S)-3-methoxypyrrolidin-1-yl]-2-methylpyrido[3,4-d]pyrimidin-4-amine
N⁴-{(1R)-1-[3-(difluoromethyl)-2-fluorophenyl]ethyl}-N⁶-[2-(dimethylamino)ethyl]-N⁶,2-dimethylpyrido[3,4-d]pyrimidine-4,6-diamine
N-{(1R)-1-[3-(difluoromethyl)-2-fluorophenyl]ethyl}-2-methyl-6-(thiomorpholin-4-yl)pyrido[3,4-d]pyrimidin-4-amine
6-[3-(difluoromethyl)azetidin-1-yl]-N-{(1R)-1-[3-(difluoromethyl)-2-fluorophenyl]ethyl}-2-methylpyrido[3,4-d]pyrimidin-4-amine
N-{(1R)-1-[3-(difluoromethyl)-2-fluorophenyl]ethyl}-6-(3,3-difluoropyrrolidin-1-yl)-2-methylpyrido[3,4-d]pyrimidin-4-amine
N-{(1R)-1-[3-(difluoromethyl)-2-fluorophenyl]ethyl}-6-(2,6-dihydropyrrolo[3,4-c]pyrazol-5(4H)-yl)-2-methylpyrido[3,4-d]pyrimidin-4-amine
1-[4-({(1R)-1-[3-(difluoromethyl)-2-fluorophenyl]ethyl}amino)-2-methylpyrido[3,4-d]pyrimidin-6-yl]piperidine-4-carbonitrile
N-{(1R)-1-[3-(difluoromethyl)-2-fluorophenyl]ethyl}-6-[hexahydrocyclopenta[c]pyrrol-2(1H)-yl]-2-methylpyrido[3,4-d]pyrimidin-4-amine (mixture of stereoisomers)
N-{(1R)-1-[3-(difluoromethyl)-2-fluorophenyl]ethyl}-6-[hexahydropyrrolo[3,4-c]pyrrol-2(1H)-yl]-2-methylpyrido[3,4-d]pyrimidin-4-amine (mixture of stereoisomers)
N-{(1R)-1-[3-(difluoromethyl)-2-fluorophenyl]ethyl}-2-methyl-6-[(3aR,6aS)-tetrahydro-1H-furo[3,4-c]pyrrol-5(3H)-yl]pyrido[3,4-d]pyrimidin-4-amine
N-{(1R)-1-[3-(difluoromethyl)-2-fluorophenyl]ethyl}-6-[(3aRS,6aRS)-hexahydro-5H-furo[2,3-c]pyrrol-5-yl]-2-methylpyrido[3,4-d]pyrimidin-4-amine (mixture of stereisomers)
N-{(1R)-1-[3-(difluoromethyl)-2-fluorophenyl]ethyl}-2-methyl-6-(2-oxa-6-azaspiro[3.4]octan-6-yl)pyrido[3,4-d]pyrimidin-4-amine
N⁶-cyclohexyl-N⁴-{(1R)-1-[3-(difluoromethyl)-2-fluorophenyl]ethyl}-N⁶,2-dimethylpyrido[3,4-d]pyrimidine-4,6-diamine
4-[4-({(1R)-1-[3-(difluoromethyl)-2-fluorophenyl]ethyl}amino)-2-methylpyrido[3,4-d]pyrimidin-6-yl]-1,4-diazepan-2-one
(3S)-1-[4-({(1R)-1-[3-(difluoromethyl)-2-fluorophenyl]ethyl}amino)-2-methylpyrido[3,4-d]pyrimidin-6-yl]pyrrolidine-3-carboxamide
(6R)-4-[4-({(1R)-1-[3-(difluoromethyl)-2-fluorophenyl]ethyl}amino)-2-methylpyrido[3,4-d]pyrimidin-6-yl]-6-methylpiperazin-2-one
(6S)-4-[4-({(1R)-1-[3-(difluoromethyl)-2-fluorophenyl]ethyl}amino)-2-methylpyrido[3,4-d]pyrimidin-6-yl]-6-methylpiperazin-2-one
N-{(1R)-1-[3-(difluoromethyl)-2-fluorophenyl]ethyl}-6-(3,3-dimethylpiperazin-1-yl)-2-methylpyrido[3,4-d]pyrimidin-4-amine
N-{(1R)-1-[3-(difluoromethyl)-2-fluorophenyl]ethyl}-2-methyl-6-(4-methyl-1,4-diazepan-1-yl)pyrido[3,4-d]pyrimidin-4-amine
N-{(1R)-1-[3-(difluoromethyl)-2-fluorophenyl]ethyl}-6-(4-ethylpiperazin-1-yl)-2-methylpyrido[3,4-d]pyrimidin-4-amine
N-{(1R)-1-[3-(difluoromethyl)-2-fluorophenyl]ethyl}-6-[(3S)-3-(dimethylamino)pyrrolidin-1-yl]-2-methylpyrido[3,4-d]pyrimidin-4-amine
N-{(1R)-1-[3-(difluoromethyl)-2-fluorophenyl]ethyl}-6-[(3R)-3-(dimethylamino)pyrrolidin-1-yl]-2-methylpyrido[3,4-d]pyrimidin-4-amine
{1-[4-({(1R)-1-[3-(difluoromethyl)-2-fluorophenyl]ethyl}amino)-2-methylpyrido[3,4-d]pyrimidin-6-yl]piperidin-4-yl}methanol
N⁴-{(1R)-1-[3-(difluoromethyl)-2-fluorophenyl]ethyl}-N⁶,2-dimethyl-N⁶-(oxan-4-yl)pyrido[3,4-d]pyrimidine-4,6-diamine
4-{[4-({(1R)-1-[3-(difluoromethyl)-2-fluorophenyl]ethyl}amino)-2-methylpyrido[3,4-d]pyrimidin-6-yl]amino}cyclohexan-1-ol (mixture of stereoisomers)
(1RS,4SR,5RS)-2-[4-({(1R)-1-[3-(difluoromethyl)-2-fluorophenyl]ethyl}amino)-2-methylpyrido[3,4-d]pyrimidin-6-yl]-2-azabicyclo[2.2.1]heptane-5-carbonitrile (mixture of stereoisomers)
N²-[4-({(1R)-1-[3-(difluoromethyl)-2-fluorophenyl]ethyl}amino)-2-methylpyrido[3,4-d]pyrimidin-6-yl]-N,N,N²-trimethylglycinamide
N-{(1R)-1-[3-(difluoromethyl)-2-fluorophenyl]ethyl}-6-(6,7-dihydropyrazolo[1,5-a]pyrazin-5(4H)-yl)-2-methylpyrido[3,4-d]pyrimidin-4-amine
N-{(1R)-1-[3-(difluoromethyl)-2-fluorophenyl]ethyl}-6-(5,6-dihydroimidazo[1,5-a]pyrazin-7(8H)-yl)-2-methylpyrido[3,4-d]pyrimidin-4-amine
N-{(1R)-1-[3-(difluoromethyl)-2-fluorophenyl]ethyl}-6-(5,6-dihydroimidazo[1,2-a]pyrazin-7(8H)-yl)-2-methylpyrido[3,4-d]pyrimidin-4-amine
N-{(1R)-1-[3-(difluoromethyl)-2-fluorophenyl]ethyl}-2-methyl-6-(1-methyl-4,6-dihydropyrrolo[3,4-c]pyrazol-5(1H)-yl)pyrido[3,4-d]pyrimidin-4-amine
N-{(1R)-1-[3-(difluoromethyl)-2-fluorophenyl]ethyl}-6-(5,6-dihydro[1,2,4]triazolo[1,5-a]pyrazin-7(8H)-yl)-2-methylpyrido[3,4-d]pyrimidin-4-amine
1-[4-({(1R)-1-[3-(difluoromethyl)-2-fluorophenyl]ethyl}amino)-2-methylpyrido[3,4-d]pyrimidin-6-yl]-4-methylpiperidine-4-carbonitrile
{4-[4-({(1R)-1-[3-(difluoromethyl)-2-fluorophenyl]ethyl}amino)-2-methylpyrido[3,4-d]pyrimidin-6-yl]piperazin-1-yl}acetonitrile
2-[4-({(1R)-1-[3-(difluoromethyl)-2-fluorophenyl]ethyl}amino)-2-methylpyrido[3,4-d]pyrimidin-6-yl]-2,6-diazaspiro[3.4]octan-5-one
2-[4-({(1R)-1-[3-(difluoromethyl)-2-fluorophenyl]ethyl}amino)-2-methylpyrido[3,4-d]pyrimidin-6-yl]-2,6-diazaspiro[3.4]octan-7-one
N-{(1R)-1-[3-(difluoromethyl)-2-fluorophenyl]ethyl}-2-methyl-6-[(3aS,6aS)-1-methylhexahydropyrrolo[3,4-b]pyrrol-5(1H)-yl]pyrido[3,4-d]pyrimidin-4-amine
N-{(1R)-1-[3-(difluoromethyl)-2-fluorophenyl]ethyl}-2-methyl-6-[(3aRS,6aSR)-5-methylhexahydropyrrolo[3,4-c]pyrrol-2(1H)-yl]pyrido[3,4-d]pyrimidin-4-amine (mixture of stereoisomers)
N-{(1R)-1-[3-(difluoromethyl)-2-fluorophenyl]ethyl}-2-methyl-6-[(3aR,6aR)-1-methylhexahydropyrrolo[3,4-b]pyrrol-5(1H)-yl]pyrido[3,4-d]pyrimidin-4-amine
N-{(1R)-1-[3-(difluoromethyl)-2-fluorophenyl]ethyl}-6-[(8aS)-hexahydropyrrolo[1,2-a]pyrazin-2(1H)-yl]-2-methylpyrido[3,4-d]pyrimidin-4-amine
N-{(1R)-1-[3-(difluoromethyl)-2-fluorophenyl]ethyl}-6-[(8aR)-hexahydropyrrolo[1,2-a]pyrazin-2(1H)-yl]-2-methylpyrido[3,4-d]pyrimidin-4-amine
N-{(1R)-1-[3-(difluoromethyl)-2-fluorophenyl]ethyl}-2-methyl-6-(6-methyl-2,6-diazaspiro[3.4]octan-2-yl)pyrido[3,4-d]pyrimidin-4-amine
6-(4-cyclopropylpiperazin-1-yl)-N-{(1R)-1-[3-(difluoromethyl)-2-fluorophenyl]ethyl}-2-methylpyrido[3,4-d]pyrimidin-4-amine
N-{(1R)-1-[3-(difluoromethyl)-2-fluorophenyl]ethyl}-2-methyl-6-(2-oxa-6-azaspiro[3.5]nonan-6-yl)pyrido[3,4-d]pyrimidin-4-amine
N-{(1R)-1-[3-(difluoromethyl)-2-fluorophenyl]ethyl}-2-methyl-6-(2-oxa-7-azaspiro[3.5]nonan-7-yl)pyrido[3,4-d]pyrimidin-4-amine
(3RS)-1-[4-({(1R)-1-[3-(difluoromethyl)-2-fluorophenyl]ethyl}amino)-2-methylpyrido[3,4-d]pyrimidin-6-yl]-3-methylpyrrolidine-3-carboxamide
1-[4-({(1R)-1-[3-(difluoromethyl)-2-fluorophenyl]ethyl}amino)-2-methylpyrido[3,4-d]pyrimidin-6-yl]piperidine-4-carboxamide
1-{4-[4-({(1R)-1-[3-(difluoromethyl)-2-fluorophenyl]ethyl}amino)-2-methylpyrido[3,4-d]pyrimidin-6-yl]piperazin-1-yl}ethan-1-one
(3R)-1-[4-({(1R)-1-[3-(difluoromethyl)-2-fluorophenyl]ethyl}amino)-2-methylpyrido[3,4-d]pyrimidin-6-yl]piperidine-3-carboxamide
(3S)-1-[4-({(1R)-1-[3-(difluoromethyl)-2-fluorophenyl]ethyl}amino)-2-methylpyrido[3,4-d]pyrimidin-6-yl]piperidine-3-carboxamide
N-{(1R)-1-[3-(difluoromethyl)-2-fluorophenyl]ethyl}-2-methyl-6-[(cis)-3,4,5-trimethylpiperazin-1-yl]pyrido[3,4-d]pyrimidin-4-amine (mixture of stereoisomers)
N-{(1R)-1-[3-(difluoromethyl)-2-fluorophenyl]ethyl}-2-methyl-6-[(3R,5R)-3,4,5-trimethylpiperazin-1-yl]pyrido[3,4-d]pyrimidin-4-amine
N-{(1R)-1-[3-(difluoromethyl)-2-fluorophenyl]ethyl}-2-methyl-6-[(3S,5S)-3,4,5-trimethylpiperazin-1-yl]pyrido[3,4-d]pyrimidin-4-amine
N-{(1R)-1-[3-(difluoromethyl)-2-fluorophenyl]ethyl}-6-[3-(dimethylamino)piperidin-1-yl]-2-methylpyrido[3,4-d]pyrimidin-4-amine (mixture of stereoisomers)
N-{(1R)-1-[3-(difluoromethyl)-2-fluorophenyl]ethyl}-6-[4-(dimethylamino)piperidin-1-yl]-2-methylpyrido[3,4-d]pyrimidin-4-amine
1-[4-({(1R)-1-[3-(difluoromethyl)-2-fluorophenyl]ethyl}amino)-2-methylpyrido[3,4-d]pyrimidin-6-yl]-3-methylpyrrolidine-3-carboxylic acid (mixture of stereoisomers)
4-{[4-({(1R)-1-[3-(difluoromethyl)-2-fluorophenyl]ethyl}amino)-2-methylpyrido[3,4-d]pyrimidin-6-yl]amino}-1-methylcyclohexan-1-ol (mixture of stereoisomers)
2-{4-[4-({(1R)-1-[3-(difluoromethyl)-2-fluorophenyl]ethyl}amino)-2-methylpyrido[3,4-d]pyrimidin-6-yl]piperazin-1-yl}ethan-1-ol
1-[4-({(1R)-1-[3-(difluoromethyl)-2-fluorophenyl]ethyl}amino)-2-methylpyrido[3,4-d]pyrimidin-6-yl]-3-(2-hydroxyethyl)pyrrolidin-3-ol (mixture of stereoisomers)
N-{(1R)-1-[3-(difluoromethyl)-2-fluorophenyl]ethyl}-2-methyl-6-(3-methyl-5,6-dihydro[1,2,4]triazolo[4,3-a]pyrazin-7(8H)-yl)pyrido[3,4-d]pyrimidin-4-amine
2-[4-({(1R)-1-[3-(difluoromethyl)-2-fluorophenyl]ethyl}amino)-2-methylpyrido[3,4-d]pyrimidin-6-yl]hexahydropyrrolo[1,2-a]pyrazin-6(2H)-one (mixture of stereoisomers)
(5RS)-7-[4-({(1R)-1-[3-(difluoromethyl)-2-fluorophenyl]ethyl}amino)-2-methylpyrido[3,4-d]pyrimidin-6-yl]-2,7-diazaspiro[4.4]nonan-3-one (mixture of stereoisomers)
6-[[1,3′-bipyrrolidin]-1′-yl]-N-{(1R)-1-[3-(difluoromethyl)-2-fluorophenyl]ethyl}-2-methylpyrido[3,4-d]pyrimidin-4-amine (mixture of stereoisomers)
7-[4-({(1R)-1-[3-(difluoromethyl)-2-fluorophenyl]ethyl}amino)-2-methylpyrido[3,4-d]pyrimidin-6-yl]hexahydro-3H-[1,3]oxazolo[3,4-a]pyrazin-3-one (mixture of stereoisomers)
1-[4-({(1R)-1-[3-(difluoromethyl)-2-fluorophenyl]ethyl}amino)-2-methylpyrido[3,4-d]pyrimidin-6-yl]-4-methyl-1,4-diazepane-2,3-dione
1-{4-[4-({(1R)-1-[3-(difluoromethyl)-2-fluorophenyl]ethyl}amino)-2-methylpyrido[3,4-d]pyrimidin-6-yl]-1,4-diazepan-1-yl}ethan-1-one
N-{(3RS)-1-[4-({(1R)-1-[3-(difluoromethyl)-2-fluorophenyl]ethyl}amino)-2-methylpyrido[3,4-d]pyrimidin-6-yl]pyrrolidin-3-yl}-N-methylacetamide (mixture of stereoisomers)
N-{1-[4-({(1R)-1-[3-(difluoromethyl)-2-fluorophenyl]ethyl}amino)-2-methylpyrido[3,4-d]pyrimidin-6-yl]piperidin-4-yl}acetamide
(3RS)-1-[4-({(1R)-1-[3-(difluoromethyl)-2-fluorophenyl]ethyl}amino)-2-methylpyrido[3,4-d]pyrimidin-6-yl]-N-methylpiperidine-3-carboxamide (mixture of stereoisomers)
2-{1-[4-({(1R)-1-[3-(difluoromethyl)-2-fluorophenyl]ethyl}amino)-2-methylpyrido[3,4-d]pyrimidin-6-yl]piperidin-4-yl}propan-2-ol
(2R)-4-[4-({(1R)-1-[3-(difluoromethyl)-2-fluorophenyl]ethyl}amino)-2-methylpyrido[3,4-d]pyrimidin-6-yl]-6-oxopiperazine-2-carboxylic acid
N-{(1R)-1-[3-(difluoromethyl)-2-fluorophenyl]ethyl}-6-[4-(2-methoxyethyl)piperazin-1-yl]-2-methylpyrido[3,4-d]pyrimidin-4-amine
5-[4-({(1R)-1-[3-(difluoromethyl)-2-fluorophenyl]ethyl}amino)-2-methylpyrido[3,4-d]pyrimidin-6-yl]-4,5,6,7-tetrahydropyrazolo[1,5-a]pyrazine-2-carbonitrile
6-[4-(2,2-difluoroethyl)piperazin-1-yl]-N-{(1R)-1-[3-(difluoromethyl)-2-fluorophenyl]ethyl}-2-methylpyrido[3,4-d]pyrimidin-4-amine
1-[5-[4-({(1R)-1-[3-(difluoromethyl)-2-fluorophenyl]ethyl}amino)-2-methylpyrido[3,4-d]pyrimidin-6-yl]hexahydropyrrolo[3,4-c]pyrrol-2(1H)-yl]ethan-1-one (mixture of stereoisomers)
N-{(1R)-1-[3-(difluoromethyl)-2-fluorophenyl]ethyl}-2-methyl-6-[3-(piperidin-1-yl)pyrrolidin-1-yl]pyrido[3,4-d]pyrimidin-4-amine (mixture of stereoisomers)
N-{(1R)-1-[3-(difluoromethyl)-2-fluorophenyl]ethyl}-2-methyl-6-[3-(morpholin-4-yl)pyrrolidin-1-yl]pyrido[3,4-d]pyrimidin-4-amine (mixture of stereoisomers)
6-[7,7-difluorohexahydropyrrolo[1,2-a]pyrazin-2(1H)-yl]-N-{(1R)-1-[3-(difluoromethyl)-2-fluorophenyl]ethyl}-2-methylpyrido[3,4-d]pyrimidin-4-amine (mixture of stereoisomers)
(3RS)-1-[4-({(1R)-1-[3-(difluoromethyl)-2-fluorophenyl]ethyl}amino)-2-methylpyrido[3,4-d]pyrimidin-6-yl]piperidine-3-sulfonamide
N-{(1R)-1-[3-(difluoromethyl)-2-fluorophenyl]ethyl}-2-methyl-6-[4-(2,2,2-trifluoroethyl)piperazin-1-yl]pyrido[3,4-d]pyrimidin-4-amine
tert-butyl {(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}carbamate
tert-butyl {3-[4-({(1R)-1-[3-(difluoromethyl)-2-fluorophenyl]ethyl}amino)-2-methylpyrido[3,4-d]pyrimidin-6-yl]-3-azabicyclo[3.1.0]hexan-1-yl}carbamate (mixture of stereoisomers)
tert-butyl {1-[4-({(1R)-1-[3-(difluoromethyl)-2-fluorophenyl]ethyl}amino)-2-methylpyrido[3,4-d]pyrimidin-6-yl]-4-fluoropyrrolidin-3-yl}carbamate (mixture of stereoisomers)
tert-butyl 6-[4-({(1R)-1-[3-(difluoromethyl)-2-fluorophenyl]ethyl}amino)-2-methylpyrido[3,4-d]pyrimidin-6-yl]-2,6-diazaspiro[3.4]octane-2-carboxylate
tert-butyl 2-[4-({(1R)-1-[3-(difluoromethyl)-2-fluorophenyl]ethyl}amino)-2-methylpyrido[3,4-d]pyrimidin-6-yl]-2,7-diazaspiro[3.5]nonane-7-carboxylate
tert-butyl 7-[4-({(1R)-1-[3-(difluoromethyl)-2-fluorophenyl]ethyl}amino)-2-methylpyrido[3,4-d]pyrimidin-6-yl]-2,7-diazaspiro[3.5]nonane-2-carboxylate
N-{(1R)-1-[3-(difluoromethyl)-2-fluorophenyl]ethyl}-2-methyl-6-(6-methyl-2,6-diazaspiro[3.4]octan-2-yl)pyrido[3,4-d]pyrimidin-4-amine
tert-butyl 2-[4-({(1R)-1-[3-(difluoromethyl)-2-fluorophenyl]ethyl}amino)-2-methylpyrido[3,4-d]pyrimidin-6-yl]-2,6-diazaspiro[3.4]octane-6-carboxylate
methyl 4-(2-{4-[4-({(1R)-1-[3-(difluoromethyl)-2-fluorophenyl]ethyl}amino)-2-methylpyrido[3,4-d]pyrimidin-6-yl]piperazin-1-yl}ethoxy)benzoate
4-(2-{4-[4-({(1R)-1-[3-(difluoromethyl)-2-fluorophenyl]ethyl}amino)-2-methylpyrido[3,4-d]pyrimidin-6-yl]piperazin-1-yl}ethoxy)benzoic acid
6-(methanesulfonyl)-2-methyl-N-{(1R)-1-[3-(trifluoromethyl)phenyl]ethyl}pyrido[3,4-d]pyrimidin-4-amine
6-[(3R)-3-aminopyrrolidin-1-yl]-N-{(1R)-1-[3-(difluoromethyl)-2-fluorophenyl]ethyl}-2-methylpyrido[3,4-d]pyrimidin-4-amine hydrochloride salt
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}cyclopropanecarboxamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-2,2-difluoroacetamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-2-methoxyacetamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}oxetane-3-carboxamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}-1-methylazetidine-3-carboxamide
methyl {(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}carbamate
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}methanesulfonamide
N-{(3R)-1-[4-({(1R)-1-[3-(difluoromethyl)-2-fluorophenyl]ethyl}amino)-2-methylpyrido[3,4-d]pyrimidin-6-yl]pyrrolidin-3-yl}cyclopropanesulfonamide
cyclopropyl{4-[4-({(1R)-1-[3-(difluoromethyl)-2-fluorophenyl]ethyl}amino)-2-methylpyrido[3,4-d]pyrimidin-6-yl]piperazin-1-yl}methanone
1-{4-[4-({(1R)-1-[3-(difluoromethyl)-2-fluorophenyl]ethyl}amino)-2-methylpyrido[3,4-d]pyrimidin-6-yl]piperazin-1-yl}-2-methoxyethan-1-one
1-{4-[4-({(1R)-1-[3-(difluoromethyl)-2-fluorophenyl]ethyl}amino)-2-methylpyrido[3,4-d]pyrimidin-6-yl]piperazin-1-yl}-2,2-difluoroethan-1-one
{4-[4-({(1R)-1-[3-(difluoromethyl)-2-fluorophenyl]ethyl}amino)-2-methylpyrido[3,4-d]pyrimidin-6-yl]piperazin-1-yl}(oxetan-3-yl)methanone
1-{4-[4-({(1R)-1-[3-(difluoromethyl)-2-fluorophenyl]ethyl}amino)-2-methylpyrido[3,4-d]pyrimidin-6-yl]piperazin-1-yl}-2-(dimethylamino)ethan-1-one
{4-[4-({(1R)-1-[3-(difluoromethyl)-2-fluorophenyl]ethyl}amino)-2-methylpyrido[3,4-d]pyrimidin-6-yl]piperazin-1-yl}(1-fluorocyclopropyl)methanone
1-{4-[4-({(1R)-1-[3-(difluoromethyl)-2-fluorophenyl]ethyl}amino)-2-methylpyrido[3,4-d]pyrimidin-6-yl]piperazin-1-yl}-2,2-difluoropropan-1-one
1-{4-[4-({(1R)-1-[3-(difluoromethyl)-2-fluorophenyl]ethyl}amino)-2-methylpyrido[3,4-d]pyrimidin-6-yl]piperazine-1-carbonyl}cyclopropane-1-carbonitrile
methyl 10-{4-[4-({(1R)-1-[3-(difluoromethyl)-2-fluorophenyl]ethyl}amino)-2-methylpyrido[3,4-d]pyrimidin-6-yl]piperazin-1-yl}-10-oxodecanoate
10-{4-[4-({(1R)-1-[3-(difluoromethyl)-2-fluorophenyl]ethyl}amino)-2-methylpyrido[3,4-d]pyrimidin-6-yl]piperazin-1-yl}-10-oxodecanoic acid
4-[4-({(1R)-1-[3-(difluoromethyl)-2-fluorophenyl]ethyl}amino)-2-methylpyrido[3,4-d]pyrimidin-6-yl]-N,N-dimethylpiperazine-1-carboxamide
N-{(1R)-1-[3-(difluoromethyl)-2-fluorophenyl]ethyl}-6-[4-(methanesulfonyl)piperazin-1-yl]-2-methylpyrido[3,4-d]pyrimidin-4-amine
2-amino-1-{4-[4-({(1R)-1-[3-(difluoromethyl)-2-fluorophenyl]ethyl}amino)-2-methylpyrido[3,4-d]pyrimidin-6-yl]piperazin-1-yl}ethan-1-one
1-{4-[4-({(1R)-1-[3-(difluoromethyl)-2-fluorophenyl]ethyl}amino)-2-methylpyrido[3,4-d]pyrimidin-6-yl]piperazin-1}-2-(methylamino)ethan-1-one
3-amino-1-{4-[4-({(1R)-1-[3-(difluoromethyl)-2-fluorophenyl]ethyl}amino)-2-methylpyrido[3,4-d]pyrimidin-6-yl]piperazin-1-yl}propan-1-one
1-{4-[4-({(1R)-1-[3-(difluoromethyl)-2-fluorophenyl]ethyl}amino)-2-methylpyrido[3,4-d]pyrimidin-6-yl]piperazin-1-yl}-3-(methylamino)propan-1-one
6-[(3R)-3-(dimethylamino)pyrrolidin-1-yl]-2-methyl-N-{(1R)-1-[3-(trifluoromethyl)phenyl]ethyl}pyrido[3,4-d]pyrimidin-4-amine
2-[2-methyl-4-({(1R)-1-[3-(trifluoromethyl)phenyl]ethyl}amino)pyrido[3,4-d]pyrimidin-6-yl]-2,6-diazaspiro[3.4]octan-7-one
1-{4-[2-methyl-4-({(1R)-1-[3-(trifluoromethyl)phenyl]ethyl}amino)pyrido[3,4-d]pyrimidin-6-yl]piperazin-1-yl}ethan-1-one
2-methyl-6-(4-methylpiperazin-1-yl)-N-{(1R)-1-[3-(trifluoromethyl)phenyl]ethyl}pyrido[3,4-d]pyrimidin-4-amine
6-fluoro-2-methyl-N-{(1R)-1-[2-methyl-3-(trifluoromethyl)phenyl]ethyl}pyrido[3,4-d]pyrimidin-4-amine
2-methyl-6-(4-methylpiperazin-1-yl)-N-{(1R)-1-[2-methyl-3-(trifluoromethyl)phenyl]ethyl}pyrido[3,4-d]pyrimidin-4-amine
2-[2-methyl-4-({(1R)-1-[2-methyl-3-(trifluoromethyl)phenyl]ethyl}amino)pyrido[3,4-d]pyrimidin-6-yl]-2,6-diazaspiro[3.4]octan-7-one
6-[(3R)-3-(dimethylamino)pyrrolidin-1-yl]-2-methyl-N-{(1R)-1-[2-methyl-3-(trifluoromethyl)phenyl]ethyl}pyrido[3,4-d]pyrimidin-4-amine
1-{4-[2-methyl-4-({(1R)-1-[2-methyl-3-(trifluoromethyl)phenyl]ethyl}amino)pyrido[3,4-d]pyrimidin-6-yl]piperazin-1-yl}ethan-1-one
2-methyl-N-{(1R)-1-[2-methyl-3-(trifluoromethyl)phenyl]ethyl}-6-(1-oxa-6-azaspiro[3.3]heptan-6-yl)pyrido[3,4-d]pyrimidin-4-amine
6-fluoro-2,8-dimethyl-N-{(1R)-1-[3-(trifluoromethyl)phenyl]ethyl}pyrido[3,4-d]pyrimidin-4-amine
1-{4-[2,8-dimethyl-4-({(1R)-1-[3-(trifluoromethyl)phenyl]ethyl}amino)pyrido[3,4-d]pyrimidin-6-yl]piperazin-1-yl}ethan-1-one
2,8-dimethyl-6-(4-methylpiperazin-1-yl)-N-{(1R)-1-[3-(trifluoromethyl)phenyl]ethyl}pyrido[3,4-d]pyrimidin-4-amine
6-[(3R)-3-(dimethylamino)pyrrolidin-1-yl]-2,8-dimethyl-N-{(1R)-1-[3-(trifluoromethyl)phenyl]ethyl}pyrido[3,4-d]pyrimidin-4-amine
2-[2,8-dimethyl-4-({(1R)-1-[3-(trifluoromethyl)phenyl]ethyl}amino)pyrido[3,4-d]pyrimidin-6-yl]-2,6-diazaspiro[3.4]octan-7-one
6-fluoro-2,8-dimethyl-N-{(1R)-1-[2-methyl-3-(trifluoromethyl)phenyl]ethyl}pyrido[3,4-d]pyrimidin-4-amine
1-{4-[2,8-dimethyl-4-({(1R)-1-[2-methyl-3-(trifluoromethyl)phenyl]ethyl}amino)pyrido[3,4-d]pyrimidin-6-yl]piperazin-1-yl}ethan-1-one
2-[2,8-dimethyl-4-({(1R)-1-[2-methyl-3-(trifluoromethyl)phenyl]ethyl}amino)pyrido[3,4-d]pyrimidin-6-yl]-2,6-diazaspiro[3.4]octan-7-one
2,8-dimethyl-6-(4-methylpiperazin-1-yl)-N-{(1R)-1-[2-methyl-3-(trifluoromethyl)phenyl]ethyl}pyrido[3,4-d]pyrimidin-4-amine
6-[(3R)-3-(dimethylamino)pyrrolidin-1-yl]-2,8-dimethyl-N-{(1R)-1-[2-methyl-3-(trifluoromethyl)phenyl]ethyl}pyrido[3,4-d]pyrimidin-4-amine
N-{(3R)-1-[2,8-dimethyl-4-({(1R)-1-[2-methyl-3-(trifluoromethyl)phenyl]ethyl}amino)pyrido[3,4-d]pyrimidin-6-yl]pyrrolidin-3-yl}acetamide
N-{(3S)-1-[2,8-dimethyl-4-({(1R)-1-[2-methyl-3-(trifluoromethyl)phenyl]ethyl}amino)pyrido[3,4-d]pyrimidin-6-yl]pyrrolidin-3-yl}acetamide
6-chloro-2-methyl-N-{(1R)-1-[3-(trifluoromethyl)phenyl]ethyl}pyrido[3,4-d]pyrimidin-4-amine
2-methyl-6-(1-methyl-1H-pyrazol-4-yl)-N-{(1R)-1-[3-(trifluoromethyl)phenyl]ethyl}pyrido[3,4-d]pyrimidin-4-amine
6-(4,5-dihydrofuran-2-yl)-2-methyl-N-{(1R)-1-[3-(trifluoromethyl)phenyl]ethyl}pyrido[3,4-d]pyrimidin-4-amine
6-(2,5-dihydrofuran-3-yl)-2-methyl-N-{(1R)-1-[3-(trifluoromethyl)phenyl]ethyl}pyrido[3,4-d]pyrimidin-4-amine
6-(3,6-dihydro-2H-pyran-4-yl)-2-methyl-N-{(1R)-1-[3-(trifluoromethyl)phenyl]ethyl}pyrido[3,4-d]pyrimidin-4-amine
6-(5,6-dihydro-2H-pyran-3-yl)-2-methyl-N-{(1R)-1-[3-(trifluoromethyl)phenyl]ethyl}pyrido[3,4-d]pyrimidin-4-amine
2-methyl-6-(1-methyl-1,2,3,6-tetrahydropyridin-4-yl)-N-{(1R)-1-[3-(trifluoromethyl)phenyl]ethyl}pyrido[3,4-d]pyrimidin-4-amine
2-methyl-6-[(3RS)-oxolan-3-yl]-N-{(1R)-1-[3-(trifluoromethyl)phenyl]ethyl}pyrido[3,4-d]pyrimidin-4-amine amine (mixture of stereoisomers)
2-methyl-6-(oxan-4-yl)-N-{(1R)-1-[3-(trifluoromethyl)phenyl]ethyl}pyrido[3,4-d]pyrimidin-4-amine
2-methyl-6-[(3RS)-oxan-3-yl]-N-{(1R)-1-[3-(trifluoromethyl)phenyl]ethyl}pyrido[3,4-d]pyrimidin-4-amine (mixture of stereoisomers)
2-methyl-6-(1-methylpiperidin-4-yl)-N-{(1R)-1-[3-(trifluoromethyl)phenyl]ethyl}pyrido[3,4-d]pyrimidin-4-amine
methyl 2-methyl-4-({(1R)-1-[3-(trifluoromethyl)phenyl]ethyl}amino)pyrido[3,4-d]pyrimidine-6-carboxylate
2-methyl-4-({(1R)-1-[3-(trifluoromethyl)phenyl]ethyl}amino)pyrido[3,4-d]pyrimidine-6-carboxamide
N,2-dimethyl-4-({(1R)-1-[3-(trifluoromethyl)phenyl]ethyl}amino)pyrido[3,4-d]pyrimidine-6-carboxamide
1-[4-({(1R)-1-[3-(difluoromethyl)-2-methylphenyl]ethyl}amino)-2-methylpyrido[3,4-d]pyrimidin-6-yl]piperidine-4-carbonitrile
N-{(1R)-1-[3-(difluoromethyl)-2-fluorophenyl]ethyl}-6-[(2S)-2,4-dimethylpiperazin-1-yl]-2-methylpyrido[3,4-d]pyrimidin-4-amine
{1-[4-({(1R)-1-[3-(difluoromethyl)-2-fluorophenyl]ethyl}amino)-2-methylpyrido[3,4-d]pyrimidin-6-yl]-4-methylpiperazin-2-yl}methanol (mixture of stereoisomers)
N-{(1R)-1-[3-(difluoromethyl)-2-fluorophenyl]ethyl}-2-methyl-6-[2-(trifluoromethyl)-5,6-dihydroimidazo[1,2-a]pyrazin-7(8H)-yl]pyrido[3,4-d]pyrimidin-4-amine
N-{(1R)-1-[3-(difluoromethyl)-2-fluorophenyl]ethyl}-2-methyl-6-[2-(trifluoromethyl)-5,6-dihydro[1,2,4]triazolo[1,5-a]pyrazin-7(8H)-yl]pyrido[3,4-d]pyrimidin-4-amine
6-(cyclobutyloxy)-N-{(1R)-1-[3-(difluoromethyl)-2-fluorophenyl]ethyl}-2-methylpyrido[3,4-d]pyrimidin-4-amine
6-butoxy-N-{(1R)-1-[3-(difluoromethyl)-2-fluorophenyl]ethyl}-2-methylpyrido[3,4-d]pyrimidin-4-amine
N-{(1R)-1-[3-(difluoromethyl)-2-fluorophenyl]ethyl}-2-methyl-6-[2-(methylamino)ethoxy]pyrido[3,4-d]pyrimidin-4-amine
N-[(1R)-1-{3-(difluoromethyl)-2-[2-(methylamino)ethoxy]phenyl}ethyl]-2-methyl-6-[2-(methylamino)ethoxy]pyrido[3,4-d]pyrimidin-4-amine
N-{(1R)-1-[3-(difluoromethyl)-2-fluorophenyl]ethyl}-2-methyl-6-[(oxetan-3-yl)oxy]pyrido[3,4-d]pyrimidin-4-amine
tert-butyl 3-{[4-({(1R)-1-[3-(difluoromethyl)-2-fluorophenyl]ethyl}amino)-2-methylpyrido[3,4-d]pyrimidin-6-yl]oxy}azetidine-1-carboxylate
N-{(1R)-1-[3-(difluoromethyl)-2-fluorophenyl]ethyl}-2-methyl-6-{[(3R)-oxolan-3-yl]oxy}pyrido[3,4-d]pyrimidin-4-amine
N-{(1R)-1-[3-(difluoromethyl)-2-{[(3R)-oxolan-3-yl]oxy}phenyl]ethyl}-2-methyl-6-{[(3R)-oxolan-3-yl]oxy}pyrido[3,4-d]pyrimidin-4-amine
N-{(1R)-1-[3-(difluoromethyl)-2-fluorophenyl]ethyl}-2-methyl-6-{[(3S)-oxolan-3-yl]oxy}pyrido[3,4-d]pyrimidin-4-amine
N-{(1R)-1-[3-(difluoromethyl)-2-{[(3S)-oxolan-3-yl]oxy}phenyl]ethyl}-2-methyl-6-{[(3S)-oxolan-3-yl]oxy}pyrido[3,4-d]pyrimidin-4-amine
N-{(1R)-1-[3-(difluoromethyl)-2-{[(3S)-1-methylpyrrolidin-3-yl]oxy}phenyl]ethyl}-2-methyl-6-{[(3S)-1-methylpyrrolidin-3-yl]oxy}pyrido[3,4-d]pyrimidin-4-amine
6-[(azetidin-3-yl)oxy]-N-{(1R)-1-[3-(difluoromethyl)-2-fluorophenyl]ethyl}-2-methylpyrido[3,4-d]pyrimidin-4-amine hydrochloride
tert-butyl {(3-trans)-1-[4-({(1R)-1-[3-(difluoromethyl)-2-fluorophenyl]ethyl}amino)-2-methylpyrido[3,4-d]pyrimidin-6-yl]-4-fluoropyrrolidin-3-yl}carbamate (mixture of stereoisomers)
6-[(trans)-3-amino-4-fluoropyrrolidin-1-yl]-N-{(1R)-1-[3-(difluoromethyl)-2-fluorophenyl]ethyl}-2-methylpyrido[3,4-d]pyrimidin-4-amine hydrochloride (mixture of stereoisomers)
tert-butyl {(cis)-1-[4-({(1R)-1-[3-(difluoromethyl)-2-fluorophenyl]ethyl}amino)-2-methylpyrido[3,4-d]pyrimidin-6-yl]-4-fluoropyrrolidin-3-yl}carbamate (mixture of stereoisomers)
6-[(cis)-3-amino-4-fluoropyrrolidin-1-yl]-N-{(1R)-1-[3-(difluoromethyl)-2-fluorophenyl]ethyl}-2-methylpyrido[3,4-d]pyrimidin-4-amine hydrochloride (mixture of stereoisomers)
or a stereoisomer, a tautomer, an N-oxide, a hydrate, a solvate, or a salt thereof, or a mixture of same.

12. A SOS1 inhibitor comprising a compound of claim 1 for use in treatment and/or prevention of cancer, wherein said compound is administered in combination with at least one other pharmacologically active substance and wherein each of said other pharmacologically active substance(s) is selected from the group consisting of: an inhibitor of HRas, NRas or KRAS and mutants thereof, in particular an inhibitor of KRAS-G12C; an inhibitor of MAP kinases, in particular MEK1, MEK2, ERK1, ERK2, ERK5 and/or of an inhibitor of PI3-kinases and mutants thereof; an inhibitor of Tropomyosin Receptor kinases and/or of mutants thereof; an inhibitor of SHP2 and mutants thereof; inhibitor of EGFR and/or of mutants thereof; an inhibitor of FGFR1 and/or FGFR2 and/or FGFR3 and/or of mutants thereof; an inhibitor of ALK and/or of mutants thereof; an inhibitor of c-MET and/or of mutants thereof; an inhibitor of BCR-ABL and/or of mutants thereof; an inhibitor of ErbB2 (Her2) and/or of mutants thereof; an inhibitor of AXL and/or of mutants thereof; an inhibitor of A-Raf and/or B-Raf and/or C-Raf and/or of mutants thereof; an inhibitor of mTOR and mutants thereof; an inhibitor of IGF1/2 and/or of IGF1-R; an inhibitor of farnesyl transferase.
